(19) United States
(12) Patent Application Publication
(10) Pub. No.: US 2017/0358756 A1
(43) Pub. Date: Dec. 14, 2017

(54) CONDENSED CYCLIC COMPOUND AND ORGANIC LIGHT-EMITTING DEVICE INCLUDING THE SAME

(71) Applicants: SAMSUNG ELECTRONICS CO., LTD., Suwon-si (KR); Samsung SDI Co., Ltd., Yongin-si (KR)

(72) Inventors: Yeonsook CHUNG, Seoul (KR); Miyoung CHAE, Suwon-si (KR); Jhunmo SON, Yongin-si (KR); Dalho HUH, Suwon-si (KR); Eunsuk KWON, Suwon-si (KR); Sangmo KIM, Hwaseong-si (KR); Hyunjung KIM, Suwon-si (KR); Saeyoun LEE, Suwon-si (KR); Soonok JEON, Seoul (KR); Yongsik JUNG, Yongin-si (KR); Joonghyuk KIM, Seoul (KR); Myungsun SIM, Suwon-si (KR)

(21) Appl. No.: 15/346,116

(22) Filed: Nov. 8, 2016

(30) Foreign Application Priority Data
Jun. 14, 2016 (KR) 10-2016-0073839

(51) Int. Cl. H01L 51/00; C07D 495/04; C09K 11/02; C07D 487/04; C07D 491/048; C07D 491/147; H01L 51/50

(52) U.S. Cl. CPC H01L 51/0072; C07D 491/048; C07D 495/04; C07D 491/147; H01L 51/0071; C09K 11/025; C07D 487/04; H01L 51/5012; H01L 51/5096

(57) ABSTRACT

A condensed cyclic compound represented by Formula 1:

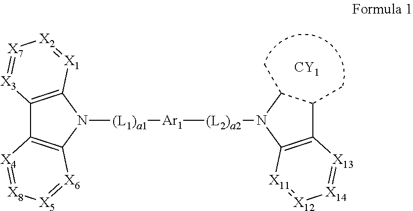

wherein, in Formula 1, groups and variables are the same as described in the specification.

### CROSS-REFERENCE TO RELATED APPLICATION

This application claims priority to Korean Patent Application No. 10-2016-0073839, filed on Jun. 14, 2016, in the Korean Intellectual Property Office, and all the benefits accruing therefrom under 35 U.S.C. §119, the content of which is incorporated herein in its entirety by reference.

### BACKGROUND

### 1. Field

One or more embodiments relate to a condensed cyclic compound and an organic light-emitting device including the same.

### 2. Description of the Related Art

Organic light-emitting devices (OLEDs) are self-emission devices that produce full-color images, and which also have wide viewing angles, high contrast ratios, and short response times, and excellent brightness, driving voltage, and response speed characteristics as compared with devices in the art.

In an example, an organic light-emitting device includes an anode, a cathode, and an organic layer disposed between the anode and the cathode, wherein the organic layer includes an emission layer. A hole transport region may be disposed between the anode and the emission layer, and an electron transport region may be disposed between the emission layer and the cathode. Holes provided from the anode may move toward the emission layer through the hole transport region, and electrons provided from the cathode may move toward the emission layer through the electron transport region. Carriers, such as holes and electrons, recombine in the emission layer to produce excitons. These excitons transition from an excited state to a ground state, thereby generating light.

Various types of organic light emitting devices are known. However, there still remains a need in OLEDs having low driving voltage, high efficiency, high brightness, and long lifespan.

### SUMMARY

One or more embodiments include a novel condensed cyclic compound and an organic light-emitting device including the same.

Additional aspects will be set forth in part in the description which follows and, in part, will be apparent from the description, or may be learned by practice of the presented embodiments.

According to one or more embodiments, a condensed cyclic compound is represented by Formula 1:

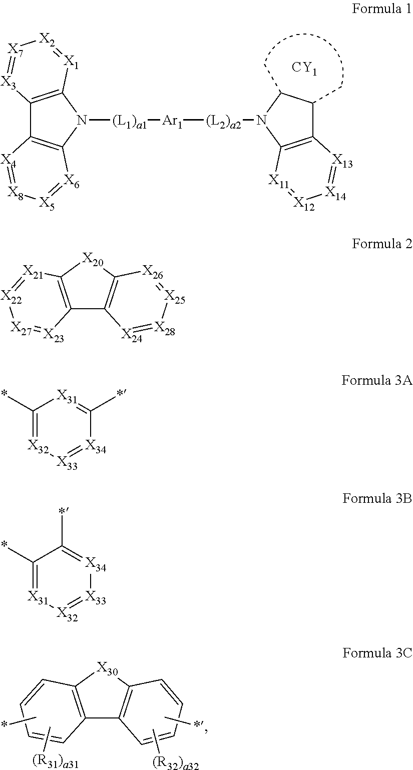

wherein, in Formulae 1, 2, and 3A to 3C,

X₁ may be N or C(R₁), X₂ may be N or C(R₂), X₃ may be N or C(R₃), X₄ may be N or C(R₄), X₅ may be N or C(R₅), X₆ may be N or C(R₆), X₇ may be N or C(R₇), and X₈ may be N or C(R₈),

X₁₁ may be N or C(R₁₁), X₁₂ may be N or C(R₁₂), X₁₃ may be N or C(R₁₃), and X₁₄ may be N or C(R₁₄),

CY₁ may be represented by Formula 2,

X₂₀ may be selected from O, S, N(R₂₀), and C(R₂₀)(R₂₉),

X₂₁ may be N or C(R₂₁), X₂₂ may be N or C(R₂₂), X₂₃ may be N or C(R₂₃), X₂₄ may be N or C(R₂₄), X₂₅ may be N or C(R₂₅), X₂₆ may be N or C(R₂₆), X₂₇ may be N or C(R₂₇), and X₂₈ may be N or C(R₂₈),

CY₁ may be fused with a neighboring 5-membered ring including N as a ring-forming atom via X₂₁ and X₂₂, X₂₂ and X₂₇, X₂₇ and X₂₃, X₂₄ and X₂₈, X₂₈ and X₂₅, or X₂₅ and X₂₆,

R₁ to R₈, R₁₁ to R₁₄, and R₂₀ to R₂₉ may each independently be selected from hydrogen, deuterium, —F, —Cl, —Br, —I, a hydroxyl group, a cyano (CN) group, a nitro group, an amino group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent non-aromatic condensed polycyclic group, a substituted or unsubstituted monovalent non-aromatic condensed heteropolycyclic group, and —Si(Q₁)(Q₂)(Q₃),

at least one selected from X₇, X₈, X₁₄, and X₂₁ to X₂₈ may be C(CN),

Ar₁ may be represented by one selected from Formulae 3A to 3C,

X₃₀ may be selected from O, S, N(R₃₀), C(R₃₀)(R₃₅), Si(R₃₀)(R₃₅), Se, and P(═O)(R₃₀),

X₃₁ may be N or C(R₃₁), X₃₂ may be N or C(R₃₂), X₃₃ may be N or C(R₃₃), and X₃₄ may be N or C(R₃₄),

R₃₀ to R₃₅ may each independently be selected from hydrogen, deuterium, a cyano group, a C₁-C₄ alkyl group, a phenyl group, a pyridinyl group, a pyrimidinyl group, a pyrazinyl group, a pyridazinyl group, a triazinyl group, and —Si(Q₁₁)(Q₁₂)(Q₁₃),

a31 and a32 may each independently be an integer selected from 0 to 3, wherein when a31 is two or more, two or more groups R₃₁ may be identical to or different from each other, and when a32 is two or more, two or more groups R₃₂ may be identical to or different from each other,

L₁ and L₂ may each independently be selected from:

a phenylene group, a pyridinylene group, a pyrimidinylene group, a pyrazinylene group, a pyridazinylene group, a triazinylene group, a dibenzofuranylene group, a dibenzothiophenylene group, and a carbazolylene group; and

a phenylene group, a pyridinylene group, a pyrimidinylene group, a pyrazinylene group, a pyridazinylene group, a triazinylene group, a dibenzofuranylene group, a dibenzothiophenylene group, and a carbazolylene group, each substituted with at least one selected from deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₁₀ alkyl group, a C₁-C₁₀ alkoxy group, a phenyl group, a naphthyl group, a pyridinyl group, a pyrimidinyl group, a pyrazinyl group, a pyridazinyl group, a triazinyl group, and —Si(Q₂₁)(Q₂₂)(Q₂₃),

a1 and a2 may each independently be an integer selected from 0 to 5, wherein a1 is two or more, two or more groups L₁ may be identical to or different from each other, and when a2 is two or more, two or more groups L₂ may be identical to or different from each other,

when Ar₁ is represented by Formula 3A or 3B, the sum of a1 and a2 is 1, and L₁ or L₂ is a phenylene group, a group represented by *-(L₁)ₐ₁-Ar₁-(L₂)ₐ₂-*′ in Formula 1 may not include a cyano (CN) group as a substituent,

* and *′ each indicate a binding site to a neighboring atom, and

at least one substituent selected from a substituent(s) of the substituted C₁-C₆₀ alkyl group, the substituted C₂-C₆₀ alkenyl group, the substituted C₂-C₆₀ alkynyl group, the substituted C₁-C₆₀ alkoxy group, the substituted C₃-C₁₀ cycloalkyl group, the substituted C₁-C₁₀ heterocycloalkyl group, the substituted C₃-C₁₀ cycloalkenyl group, the substituted C₁-C₁₀ heterocycloalkenyl group, the substituted C₆-C₆₀ aryl group, the substituted C₆-C₆₀ aryloxy group, the substituted C₆-C₆₀ arylthio group, the substituted C₁-C₆₀ heteroaryl group, the substituted monovalent non-aromatic condensed polycyclic group, and the substituted monovalent non-aromatic condensed heteropolycyclic group may be selected from deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, and —Si(Q₃₁)(Q₃₂)(Q₃₃),

wherein Q₁ to Q₃, Q₁₁ to Q₁₃, Q₂₁ to Q₂₃, and Q₃₁ to Q₃₃ may each independently be selected from hydrogen, a C₁-C₆₀ alkyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₁-C₆₀ heteroaryl group, a monovalent non-aromatic condensed polycyclic group, and a monovalent non-aromatic condensed heteropolycyclic group.

### BRIEF DESCRIPTION OF THE DRAWING

These and/or other aspects will become apparent and more readily appreciated from the following description of the embodiments, taken in conjunction with the accompanying drawing in which:

FIG. 1 is a schematic view of an organic light-emitting device according to an embodiment.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description. As used herein, the term “and/or” includes any and all combinations of one or more of the associated listed items. Expressions such as “at least one of,” when preceding a list of elements, modify the entire list of elements and do not modify the individual elements of the list.

It will be understood that when an element is referred to as being “on” another element, it can be directly in contact with the other element or intervening elements may be present therebetween. In contrast, when an element is referred to as being “directly on” another element, there are no intervening elements present.

It will be understood that, although the terms first, second, third etc. may be used herein to describe various elements, components, regions, layers, and/or sections, these elements, components, regions, layers, and/or sections should not be limited by these terms. These terms are only used to distinguish one element, component, region, layer, or section from another element, component, region, layer, or section. Thus, a first element, component, region, layer, or section discussed below could be termed a second element, component, region, layer, or section without departing from the teachings of the present embodiments.

The terminology used herein is for the purpose of describing particular embodiments only and is not intended to be limiting. As used herein, the singular forms “a,” “an,” and “the” are intended to include the plural forms as well, unless the context clearly indicates otherwise.

The term “or” means “and/or.” It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

Unless otherwise defined, all terms (including technical and scientific terms) used herein have the same meaning as commonly understood by one of ordinary skill in the art to which this general inventive concept belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

Exemplary embodiments are described herein with reference to cross section illustrations that are schematic illustrations of idealized embodiments. As such, variations from the shapes of the illustrations as a result, for example, of manufacturing techniques and/or tolerances, are to be expected. Thus, embodiments described herein should not be construed as limited to the particular shapes of regions as illustrated herein but are to include deviations in shapes that result, for example, from manufacturing. For example, a region illustrated or described as flat may, typically, have rough and/or nonlinear features. Moreover, sharp angles that are illustrated may be rounded. Thus, the regions illustrated in the figures are schematic in nature and their shapes are not intended to illustrate the precise shape of a region and are not intended to limit the scope of the present claims.

“About” or “approximately” as used herein is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” can mean within one or more standard deviations, or within +30%, 20%, 10%, 5% of the stated value.

According to an aspect of the present disclosure, a condensed cyclic compound according to an embodiment may be represented by Formula 1:

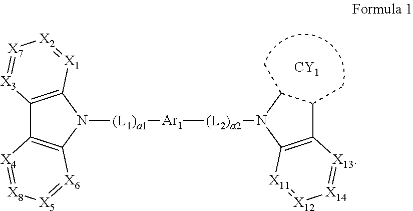

In Formula 1,

X₁ may be N or C(R₁), X₂ may be N or C(R₂), X₃ may be N or C(R₃), X₄ may be N or C(R₄), X₅ may be N or C(R₅), X₆ may be N or C(R₆), X₇ may be N or C(R₇), and X₈ may be N or C(R₈),

X₁₁ may be N or C(R₁₁), X₁₂ may be N or C(R₁₂), X₁₃ may be N or C(R₁₃), and X₁₄ may be N or C(R₁₄). R₁ to R₉ and R₁₁ to R₁₄ are the same as described below.

In one or more embodiments, in Formula 1, X₇ may be C(R₇), X₈ may be C(R₈), and X₁₄ may be C(R₁₄).

In one or more embodiments, all of X₁ to X₆ and X₁₁ to X₁₃ in Formula 1 may not be N.

In one or more embodiments, in Formula 1, one selected from X₁ to X₆ and X₁₁ to X₁₃ may be N, and the others may not be N.

CY₁ in Formula 1 may be represented by Formula 2:

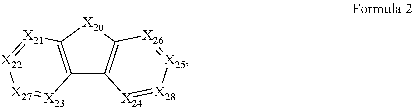

wherein, X₂₀ in Formula 2 may be selected from O, S, N(R₂₀), and C(R₂₀)(R₂₉).

X₂₁ may be N or C(R₂₁), X₂₂ may be N or C(R₂₂), X₂₃ may be N or C(R₂₃), X₂₄ may be N or C(R₂₄), X₂₅ may be N or C(R₂₅), X₂₆ may be N or C(R₂₆), X₂₇ may be N or C(R₂₇), and X₂₈ may be N or C(R₂₈). R₂₀ to R₂₉ are the same as described below.

CY₁ may be fused with a neighboring 5-membered ring including N as a ring-forming atom via X₂₁ and X₂₂, X₂₂ and X₂₇, X₂₇ and X₂₃, X₂₄ and X₂₈, X₂₈ and X₂₅, or X₂₅ and X₂₆.

In one or more embodiments, in Formula 2, X₂₇ may be C(R₂₇), or X₂₈ may be C(R₂₈).

In one or more embodiments, all of X₂₁ to X₂₆ in Formula 2 may not be N.

In one or more embodiments, in Formula 2, one selected from X₂₁ to X₂₆ may be N, and the others may not be N.

R₁ to R₈, R₁₁ to R₁₄, and R₂₀ to R₂₉ in Formulae 1 and 2 may each independently be selected from hydrogen, deuterium, —F, —Cl, —Br, —I, a hydroxyl group, a cyano (CN) group, a nitro group, an amino group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent non-aromatic condensed polycyclic group, a substituted or unsubstituted monovalent non-aromatic condensed heteropolycyclic group, and —Si(Q₁)(Q₂)(Q₃). Q₁ to Q₃ are the same as described below.

In one or more embodiments, in Formulae 1 and 2,

R₁ to R₈, R₁₁ to R₁₄, and R₂₀ to R₂₉ may each independently be selected from:

hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, and a C₁-C₂₀ alkoxy group;

a C₁-C₂₀ alkyl group and a C₁-C₂₀ alkoxy group, each substituted with at least one selected from deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a phenyl group, a naphthyl group, a pyridinyl group, a pyrimidinyl group, a pyrazinyl group, a pyridazinyl group, and a triazinyl group;

a cyclopentyl group, a cyclohexyl group, a cyclopentenyl group, a cyclohexenyl group, a cycloheptenyl group, a phenyl group, a pentalenyl group, an indenyl group, a naphthyl group, an azulenyl group, a heptalenyl group, an indacenyl group, an acenaphthyl group, a fluorenyl group, a spiro-fluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a perylenyl group, a pentaphenyl group, a hexacenyl group, a pyrrolyl group, an imidazolyl group, a pyrazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a phenanthridinyl group, an acridinyl group, a phenanthrolinyl group, a phenazinyl group, a benzoxazolyl group, a benzimidazolyl group, a furanyl group, a benzofuranyl group, a thiophenyl group, a benzothiophenyl group, a thiazolyl group, an isothiazolyl group, a benzothiazolyl group, an isoxazolyl group, an oxazolyl group, a triazolyl group, a tetrazolyl group, an oxadiazolyl group, a triazinyl group, a dibenzofuranyl group, a dibenzothiophenyl group, an imidazopyridimidinyl group, and an imidazopyridinyl group;

a cyclopentyl group, a cyclohexyl group, a cyclopentenyl group, a cyclohexenyl group, a cycloheptenyl group, a phenyl group, a pentalenyl group, an indenyl group, a naphthyl group, an azulenyl group, a heptalenyl group, an indacenyl group, an acenaphthyl group, a fluorenyl group, a spiro-fluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a perylenyl group, a pentaphenyl group, a hexacenyl group, a pyrrolyl group, an imidazolyl group, a pyrazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a phenanthridinyl group, an acridinyl group, a phenanthrolinyl group, a phenazinyl group, a benzoxazolyl group, a benzimidazolyl group, a furanyl group, a benzofuranyl group, a thiophenyl group, a benzothiophenyl group, a thiazolyl group, an isothiazolyl group, a benzothiazolyl group, an isoxazolyl group, an oxazolyl group, a triazolyl group, a tetrazolyl group, an oxadiazolyl group, a triazinyl group, a dibenzofuranyl group, a dibenzothiophenyl group, an imidazopyridimidinyl group, and an imidazopyridinyl group, each substituted with at least one selected from deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₂-C₂₀ alkenyl group, a C₂-C₂₀ alkynyl group, a C₁-C₂₀ alkoxy group, a phenyl group, a naphthyl group, an anthracenyl group, a pyrenyl group, a phenanthrenyl group, a fluorenyl group, a pyridinyl group, a pyrimidinyl group, a pyrazinyl group, a pyridazinyl group, a triazinyl group, a quinolinyl group, an isoquinolinyl group, a phthalazinyl group, a quinoxalinyl group, a cinnolinyl group, a quinazolinyl group, and —Si(Q₃₁)(Q₃₂)(Q₃₃); and

—Si(Q₁)(Q₂)(Q₃),

wherein Q₁ to Q₃ and Q₃₁ to Q₃₃ may each independently be selected from hydrogen, a C₁-C₂₀ alkyl group, a C₁-C₂₀ alkoxy group, a phenyl group, a naphthyl group, an anthracenyl group, a pyrenyl group, a phenanthrenyl group, a fluorenyl group, a pyridinyl group, a pyrimidinyl group, a pyrazinyl group, a pyridazinyl group, a triazinyl group, a quinolinyl group, an isoquinolinyl group, a phthalazinyl group, a quinoxalinyl group, a cinnolinyl group, and a quinazolinyl group.

In one or more embodiments, in Formulae 1 and 2,

R₁ to R₆, R₁₁ to R₁₃, R₂₀ to R₂₆, and R₂₉ may each independently be selected from:

hydrogen, deuterium, —F, —Cl, —Br, —I, a hydroxyl group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₁₀ alkyl group, and a C₁-C₁₀ alkoxy group; a phenyl group, a naphthyl group, a pyridinyl group, a pyrimidinyl group, a pyrazinyl group, a pyridazinyl group, and a triazinyl group;

a phenyl group, a naphthyl group, a pyridinyl group, a pyrimidinyl group, a pyrazinyl group, a pyridazinyl group, and a triazinyl group, each substituted with at least one selected from deuterium, —F, —Cl, —Br, —I, a hydroxyl group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₁₀ alkyl group, a C₁-C₁₀ alkoxy group, a phenyl group, a naphthyl group, a pyridinyl group, a pyrimidinyl group, a pyrazinyl group, a pyridazinyl group, a triazinyl group, and —Si(Q₃₁)(Q₃₂)(Q₃₃); and

—Si(Q₁)(Q₂)(Q₃),

R₇, R₈, R₁₄, R₂₇, and R₂₈ may each independently be selected from:

hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₁₀ alkyl group, and a C₁-C₁₀ alkoxy group;

a phenyl group, a naphthyl group, a pyridinyl group, a pyrimidinyl group, a pyrazinyl group, a pyridazinyl group, and a triazinyl group;

a phenyl group, a naphthyl group, a pyridinyl group, a pyrimidinyl group, a pyrazinyl group, a pyridazinyl group, and a triazinyl group, each substituted with at least one selected from deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₁₀ alkyl group, a C₁-C₁₀ alkoxy group, a phenyl group, a naphthyl group, a pyridinyl group, a pyrimidinyl group, a pyrazinyl group, a pyridazinyl group, a triazinyl group, and —Si(Q₃₁)(Q₃₂)(Q₃₃); and

—Si(Q₁)(Q₂)(Q₃),

wherein Q₁ to Q₃ and Q₃₁ to Q₃₃ may each independently be selected from hydrogen, a C₁-C₁₀ alkyl group, a C₁-C₁₀ alkoxy group, a phenyl group, a naphthyl group, a pyridinyl group, a pyrimidinyl group, a pyrazinyl group, a pyridazinyl group, and a triazinyl group.

In one or more embodiments, R₁ to R₈, R₁₁ to R₁₄, and R₂₀ to R₂₉ in Formulae 1 and 2 may each independently be selected from hydrogen, deuterium, a cyano group, a methyl group, an ethyl group, an n-propyl group, an iso-propyl group, an n-butyl group, an iso-butyl group, a sec-butyl group, a tert-butyl group, an n-pentyl group, an iso-pentyl group, a sec-pentyl group, a tert-pentyl group, an n-hexyl group, an iso-hexyl group, a sec-hexyl group, a tert-hexyl group, and —Si(Q₁)(Q₂)(Q₃),

wherein Q₁ to Q₃ may each independently be selected from hydrogen, a C₁-C₁₀ alkyl group, a C₁-C₁₀ alkoxy group, and a phenyl group, but embodiments are not limited thereto.

At least one selected from X₇, X₈, X₁₄, and X₂₁ to X₂₈ in Formulae 1 and 2 may be C(CN).

In one or more embodiments, at least two selected from X₇, X₈, X₁₄, and X₂₁ to X₂₈ in Formulae 1 and 2 may be C(CN).

In one or more embodiments, at least one selected from X₇, X₈, X₁₄, X₂₁, and X₂₆ to X₂₈ in Formulae 1 and 2 may be C(CN).

In one or more embodiments, at least one selected from X₇ and X₈ in Formula 1 may be C(CN).

In one or more embodiments, X₁ to X₆, X₁₁ to X₁₃, and X₂₂ to X₂₅ in Formulae 1 and 2 may not be C(CN), but embodiments are not limited thereto.

Ar₁ in Formula 1 may be represented by one selected from Formulae 3A to 3C:

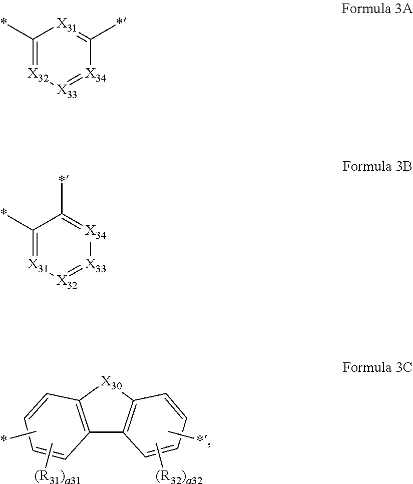

wherein, in Formulae 3A to 3C, X₃₀ may be O, S, N(R₃₀), C(R₃₀)(R₃₅), Si(R₃₀)(R₃₅), Se, and P(═O)(R₃₀).

In one or more embodiments, X₃₀ in Formulae 3A to 3C may be selected from O, S, N(R₃₀), and C(R₃₀)(R₃₅).

In Formulae 3A to 3C, X₃₁ may be N or C(R₃₁), X₃₂ may be N or C(R₃₂), X₃₃ may be N or C(R₃₃), and X₃₄ may be N or C(R₃₄).

R₃₀ to R₃₅ in Formulae 3A to 3C may each independently be selected from hydrogen, deuterium, a cyano group, a C₁-C₄ alkyl group, a phenyl group, a pyridinyl group, a pyrimidinyl group, a pyrazinyl group, a pyridazinyl group, a triazinyl group, and —Si(Q₁₁)(Q₁₂)(Q₁₃). Q₁₁ to Q₁₃ are the same as described below.

In one or more embodiments, in Formulae 3A to 3C,

R₃₁ to R₃₄ may each independently be selected from hydrogen, deuterium, a cyano group, a C₁-C₄ alkyl group, a phenyl group, a pyridinyl group, a pyrimidinyl group, a pyrazinyl group, a pyridazinyl group, a triazinyl group, and —Si(Q₁₁)(Q₁₂)(Q₁₃), and

R₃₀ and R₃₅ may each independently be selected from a C₁-C₄ alkyl group, a phenyl group, a pyridinyl group, a pyrimidinyl group, a pyrazinyl group, a pyridazinyl group, and a triazinyl group.

a31 and a32 in Formulae 3A to 3C respectively indicate the number of groups R₃₁ and the number of groups R₃₂, and a31 and a32 may each independently be an integer selected from 0 to 3. In Formulae 3A to 3C, when a31 is two or more, two or more groups R₃₁ may be identical to or different from each other, and when a32 is two or more, two or more groups R₃₂ may be identical to or different from each other.

In one or more embodiments, a31 and a32 in Formulae 3A to 3C may each independently be 0, 1, or 2.

Ar₁ in Formula 1 may be represented by one selected from Formulae 3A-1 to 3A-10, 3B-1 to 3B-8, and 3C-1 to 3C-9, but is not limited thereto:

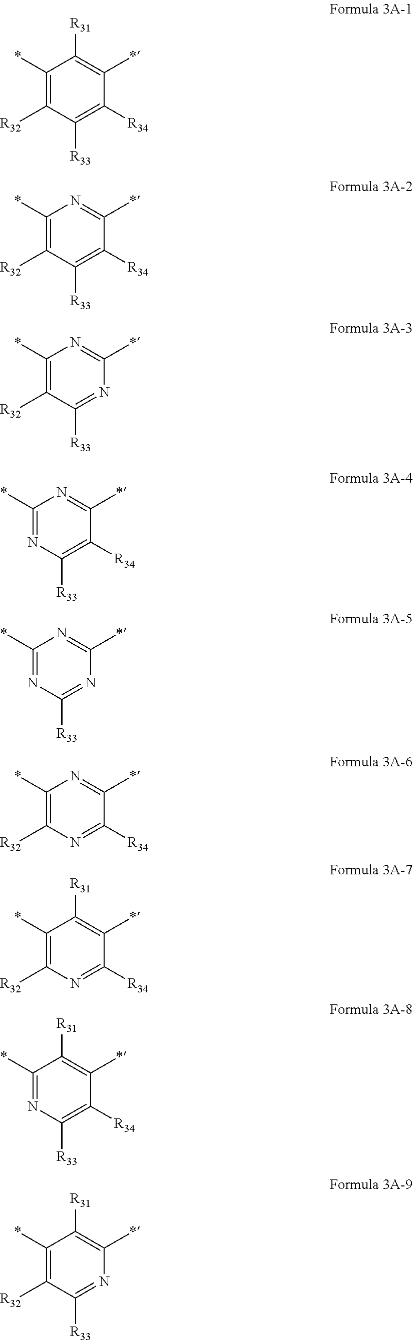

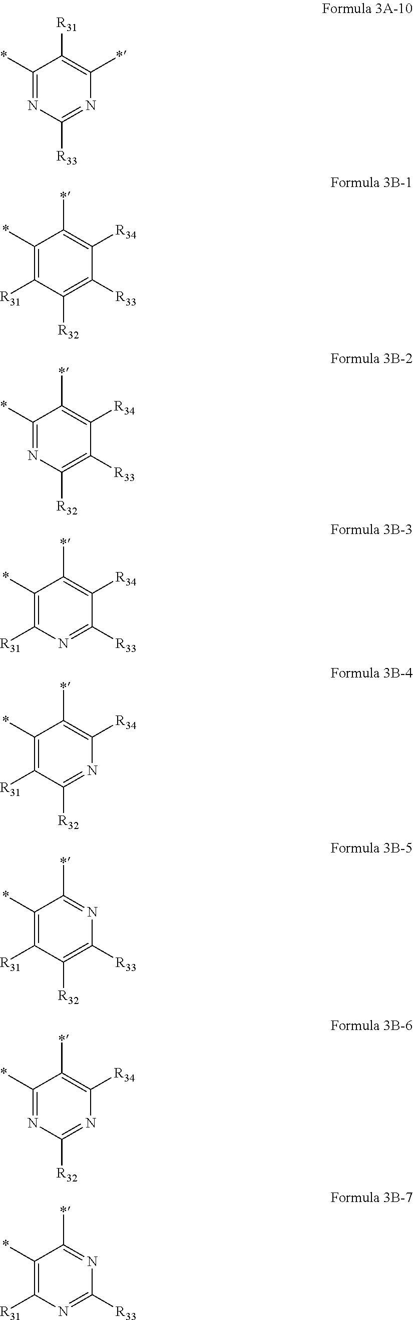

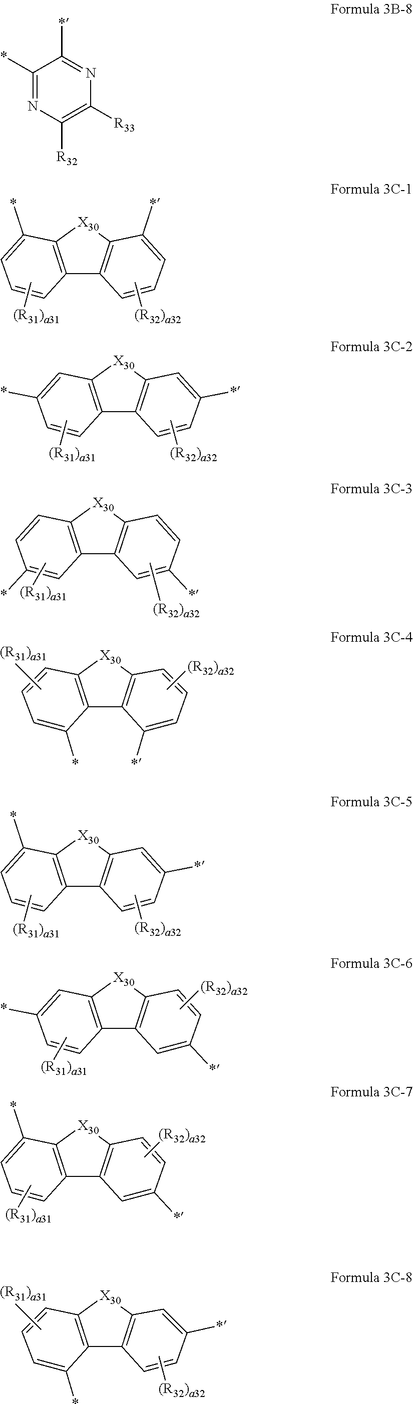

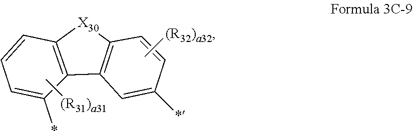

wherein, in Formulae 3A-1 to 3A-10, 3B-1 to 3B-8, and 3C-1 to 3C-9,

X₃₀ may be selected from O, S, N(R₃₀), C(R₃₀)(R₃₅), Si(R₃₀)(R₃₅), Se, and P(═O)(R₃₀),

R₃₁ to R₃₄ may each independently be selected from hydrogen, deuterium, a cyano group, a methyl group, an ethyl group, an n-propyl group, an iso-propyl group, an n-butyl group, an iso-butyl group, a sec-butyl group, a tert-butyl group, a phenyl group, a pyridinyl group, a pyrimidinyl group, a pyrazinyl group, a pyridazinyl group, a triazinyl group, and —Si(Q₁₁)(Q₁₂)(Q₁₃),

R₃₀ and R₃₅ may each independently be selected from hydrogen, deuterium, a cyano group, a methyl group, an ethyl group, an n-propyl group, an iso-propyl group, an n-butyl group, an iso-butyl group, a sec-butyl group, a tert-butyl group, a phenyl group, a pyridinyl group, a pyrimidinyl group, a pyrazinyl group, a pyridazinyl group, and a triazinyl group,

Q₁₁ to Q₁₃ may each independently be selected from hydrogen, a C₁-C₁₀ alkyl group, a C₁-C₁₀ alkoxy group, and a phenyl group,

a31 and a32 may each independently be 0 or 1, and

* and *′ each indicate a binding site to a neighboring atom.

In one or more embodiments, Ar₁ in Formula 1 may be selected from Formulae 3A-1 to 3A-10, 3B-1 to 3B-8, and 3C-3, but is not limited thereto.

In one or more embodiments,

i) in Formulae 1 and 2, one selected from X₇, X₈, X₁₄, X₂₇, and X₂₈ may be C(CN), the others may not be C(CN), and Ar₁ may be represented by one selected from Formulae 3A-1 to 3A-10, 3B-1 to 3B-8, and 3C-3; or

ii) in Formulae 1 and 2, at least two selected from X₇, X₈, X₁₄, X₂₇, and X₂₈ may be C(CN), the others may not be C(CN), and Ar₁ may be represented by one selected from Formulae 3A-1 to 3A-10, 3B-1 to 3B-8, and 3C-3, but embodiments are not limited thereto.

L₁ and L₂ in Formula 1 may each independently be selected from:

a phenylene group, a pyridinylene group, a pyrimidinylene group, a pyrazinylene group, a pyridazinylene group, a triazinylene group, a dibenzofuranylene group, a dibenzothiophenylene group, and a carbazolylene group; and

a phenylene group, a pyridinylene group, a pyrimidinylene group, a pyrazinylene group, a pyridazinylene group, a triazinylene group, a dibenzofuranylene group, a dibenzothiophenylene group, and a carbazolylene group, each substituted with at least one selected from deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₁₀ alkyl group, a C₁-C₁₀ alkoxy group, a phenyl group, a naphthyl group, a pyridinyl group, a pyrimidinyl group, a pyrazinyl group, a pyridazinyl group, a triazinyl group, and —Si(Q₂₁)(Q₂₂)(Q₂₃).

In one or more embodiments, L₁ and L₂ in Formula 1 may each independently be selected from:

a phenylene group, a pyridinylene group, a pyrimidinylene group, and a triazinylene group; and

a phenylene group, a pyridinylene group, a pyrimidinylene group, and a triazinylene group, each substituted with at least one selected from deuterium, a cyano group, a methyl group, an ethyl group, an n-propyl group, an iso-propyl group, an n-butyl group, an iso-butyl group, a sec-butyl group, a tert-butyl group, a phenyl group, a pyridinyl group, a pyrimidinyl group, a pyrazinyl group, a pyridazinyl group, a triazinyl group, and —Si(Q₂₁)(Q₂₂)(Q₂₃).

a1 and a2 in Formula 1 may each independently be an integer selected from 0 to 5. a1 indicates the number of groups L₁, wherein when a1 is zero, *-(L₁)ₐ₁-*′ may be a single bond. When a1 is two or more, two or more groups L₁ may be identical to or different from each other. a2 indicates the number of groups L₂, wherein when a2 is zero, *-(L₂)ₐ₂-*′ may be a single bond. When a2 is two or more, two or more groups L₂ may be identical to or different from each other.

In one or more embodiments, a1 and a2 may each independently be 0, 1, or 2.

In one or more embodiments, a1 and a2 may each independently be 0 or 1.

In one or more embodiments, L₁ and L₂ in Formula 1 may each independently be selected from:

a phenylene group; and

a phenylene group substituted with at least one selected from deuterium, a cyano group, a methyl group, an ethyl group, an n-propyl group, an iso-propyl group, an n-butyl group, an iso-butyl group, a sec-butyl group, a tert-butyl group, a phenyl group, a pyridinyl group, a pyrimidinyl group, a pyrazinyl group, a pyridazinyl group, a triazinyl group, and —Si(Q₂₁)(Q₂₂)(Q₂₃),

wherein Q₂₁ to Q₂₃ may each independently be selected from hydrogen, a C₁-C₁₀ alkyl group, a C₁-C₁₀ alkoxy group, and a phenyl group, and

a1 and a2 may each independently be 0 or 1.

When Ar₁ is represented by Formula 3A or 3B, the sum of a1 and a2 is 1, and L₁ or L₂ is a phenylene group, a group represented by *-(L₁)ₐ₁-Ar₁-(L₂)ₐ₂-*′ in Formula 1 may not include a cyano (CN) group as a substituent.

In one or more embodiments, when Ar₁ in Formula 1 is represented by Formula 3A-1 or 3B-1, the sum of a1 and a2 is 1, and L₁ or L₂ are a phenylene group, a group represented by *-(L₁)ₐ₁-Ar₁-(L₂)ₐ₂-*′ in Formula 1 may not include a cyano group as a substituent.

In one or more embodiments, in Formula 1, a1 and a2 may be zero, and Ar₁ may be selected from groups represented by Formulae 3A-1, 3A-2, 3B-1, and 3C-3.

In one or more embodiments, the sum of a1 and a2 in Formula 1 may be one or more, and *-(L₁)ₐ₁-Ar₁-(L₂)ₐ₂-*′ may be represented by one selected from Formulae 3-1 to 3-57, but embodiments are not limited thereto:

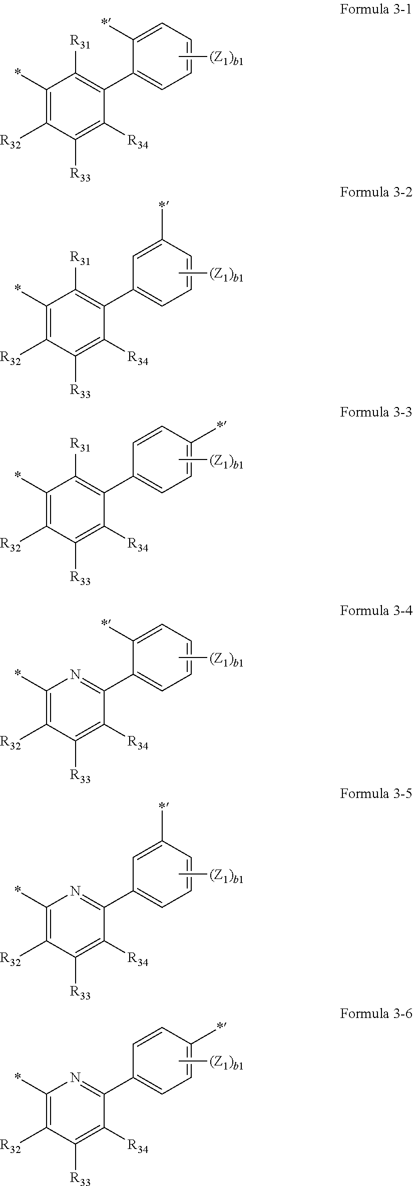

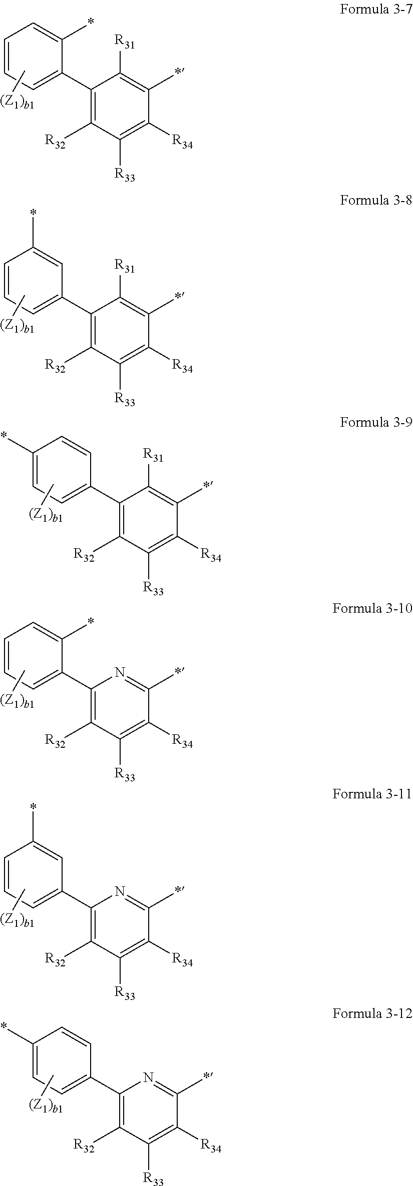

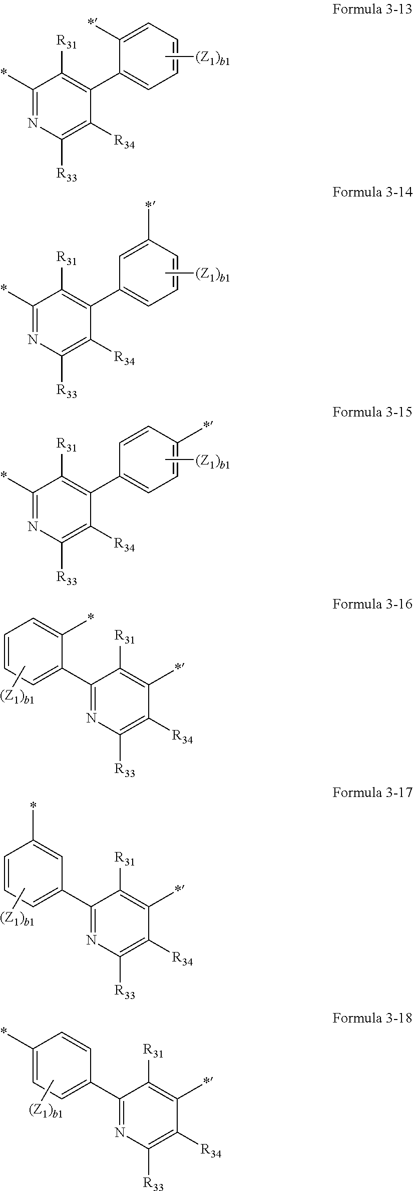

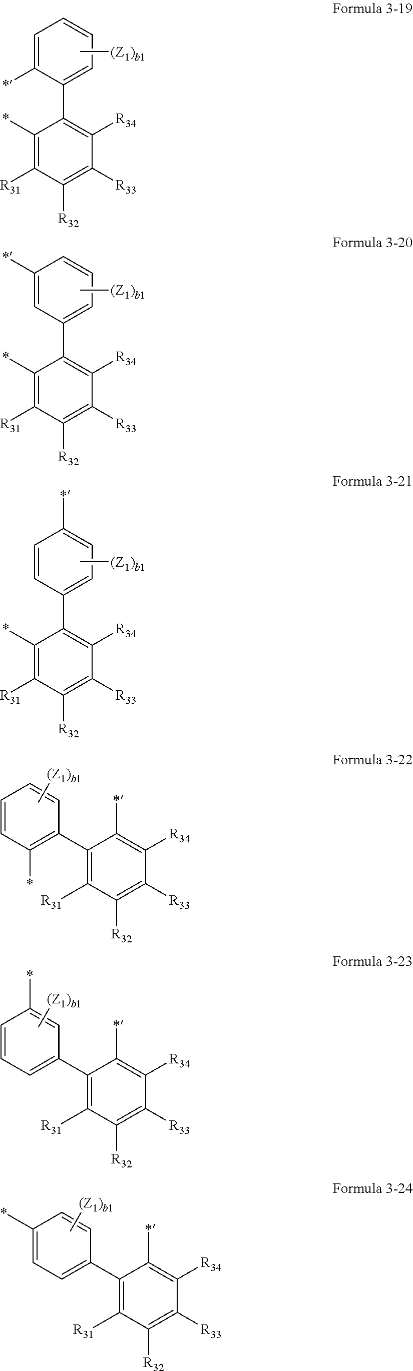

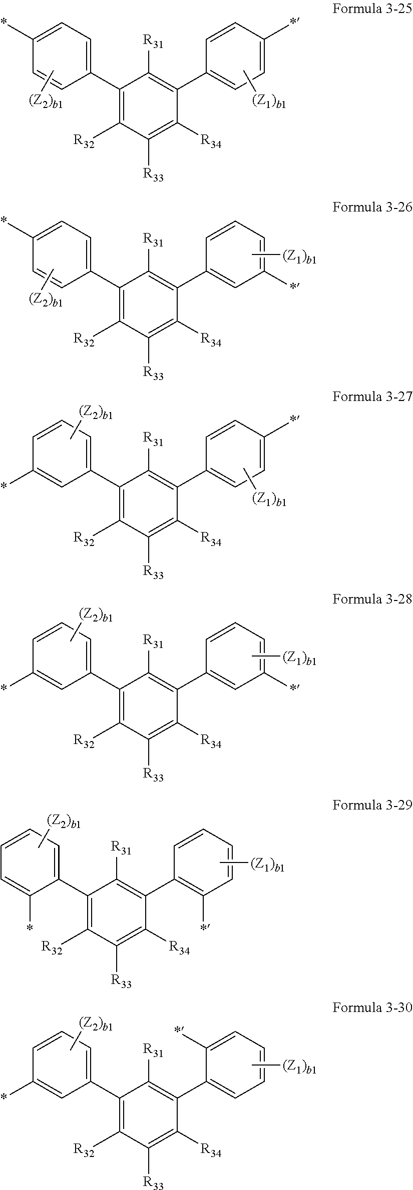

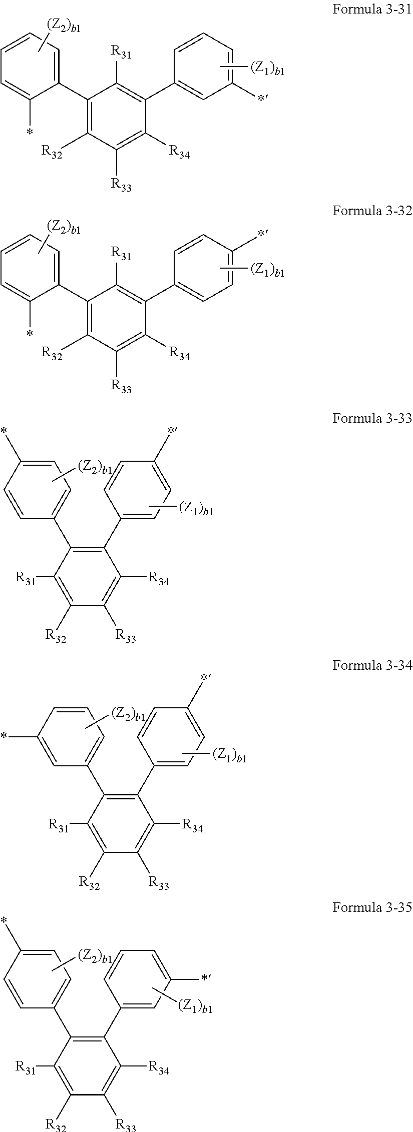

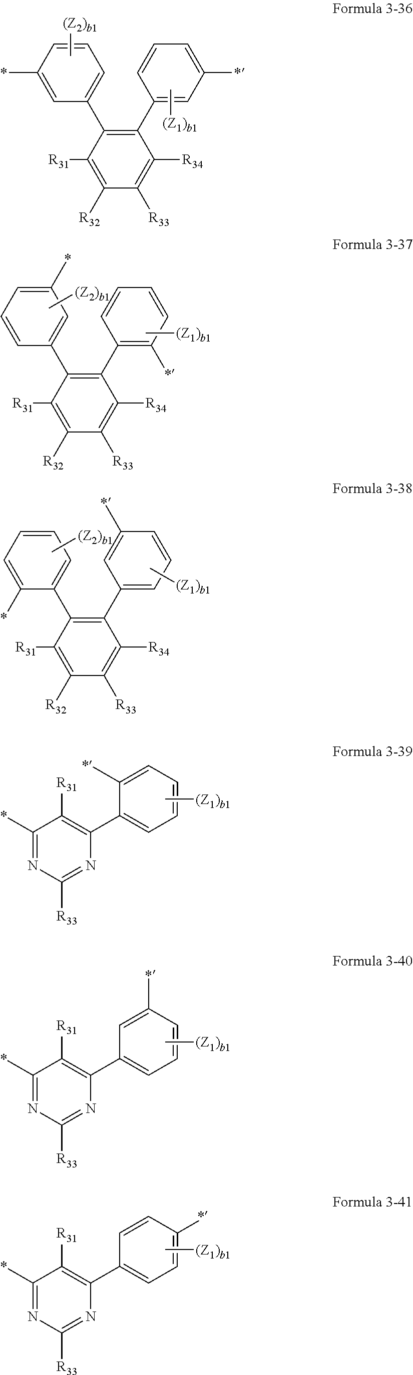

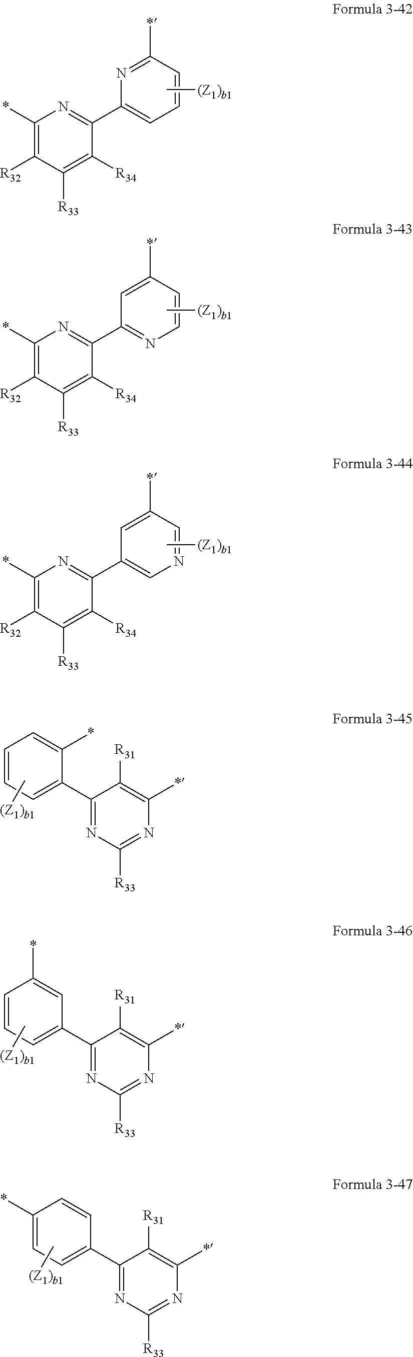

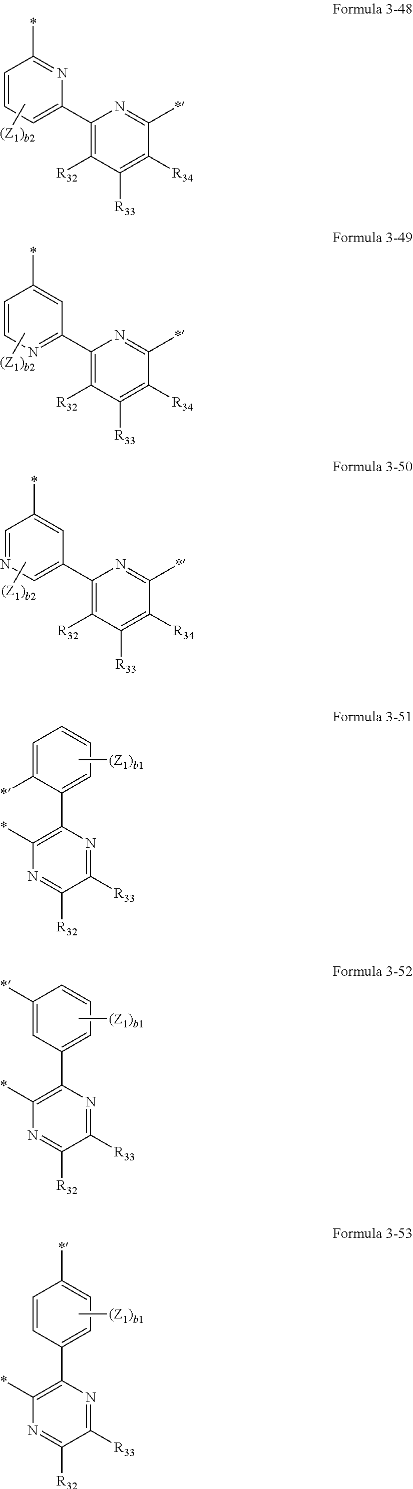

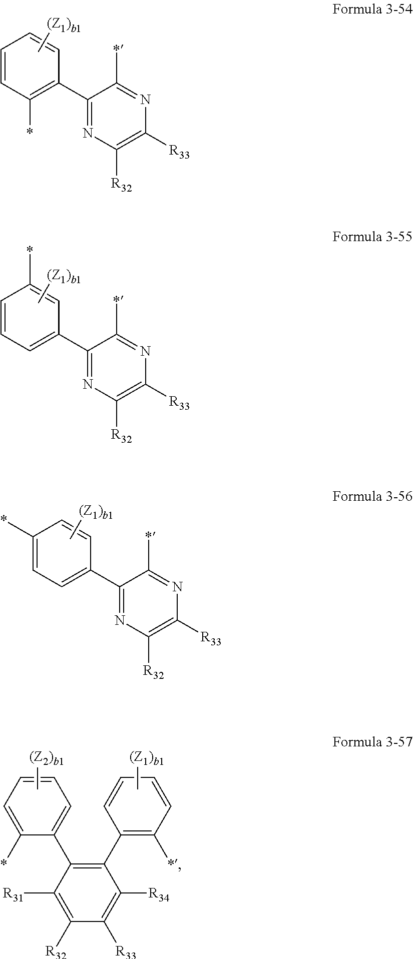

wherein, in Formulae 3-1 to 3-57,

R₃₁ to R₃₄, Z₁, and Z₂ may each independently be selected from hydrogen, deuterium, a cyano group, a methyl group, an ethyl group, an n-propyl group, an iso-propyl group, an n-butyl group, an iso-butyl group, a sec-butyl group, a tert-butyl group, a phenyl group, a pyridinyl group, a pyrimidinyl group, a pyrazinyl group, a pyridazinyl group, a triazinyl group, and —Si(Q₁₁)(Q₁₂)(Q₁₃),

wherein Q₁₁ to Q₁₃ may each independently be selected from hydrogen, a C₁-C₁₀ alkyl group, a C₁-C₁₀ alkoxy group, and a phenyl group,

b1 may be an integer selected from 0 to 4, and b2 may be an integer selected from 0 to 3, and

* and *′ each indicate a binding site to a neighboring nitrogen atom.

In one or more embodiments, the condensed cyclic compound may be represented by one selected from Formulae 1(1) to 1(7):

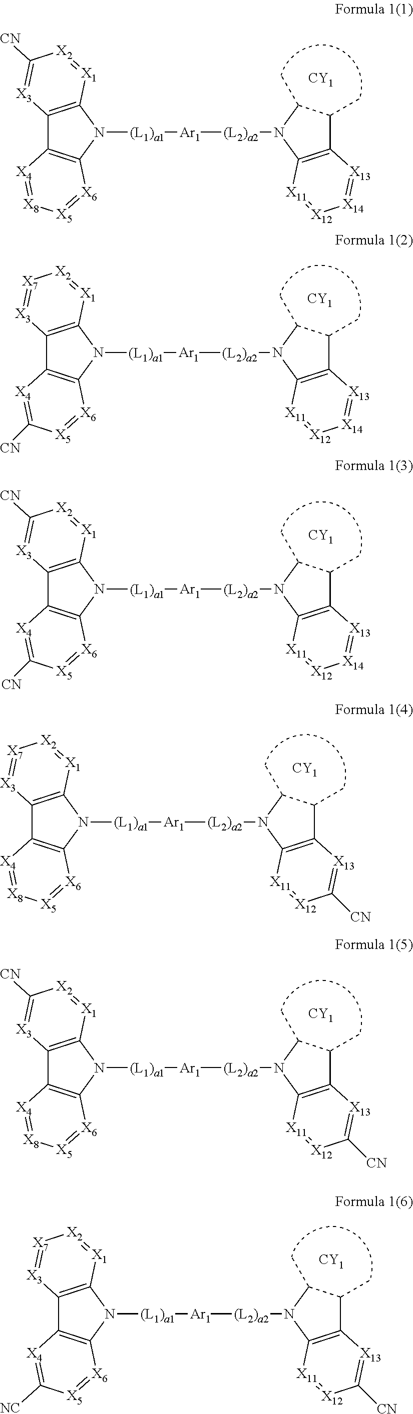

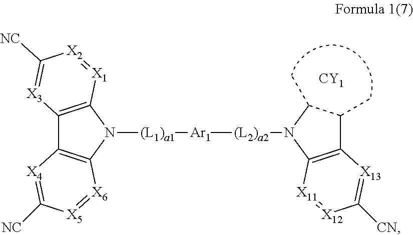

wherein X₁ to X₈, X₁₁ to X₁₄, CY₁, Ar₁, L₁, L₂, a1, and a2 in Formulae 1(1) to 1(7) are the same as described above.

In one or more embodiments, X₁ may be N or C(R₁), X₂ may be N or C(R₂), X₃ may be N or C(R₃), X₄ may be N or C(R₄), X₅ may be N or C(R₅), X₆ may be N or C(R₆), X₁₁ may be N or C(R₁₁), X₁₂ may be N or C(R₁₂), X₁₃ may be N or C(R₁₃), and X₁ to X₈ and X₁₁ to X₁₃ may not be C(CN).

In one or more embodiments, the condensed cyclic compound represented by Formula 1 may be represented by one selected from Formulae 1A to 1F:

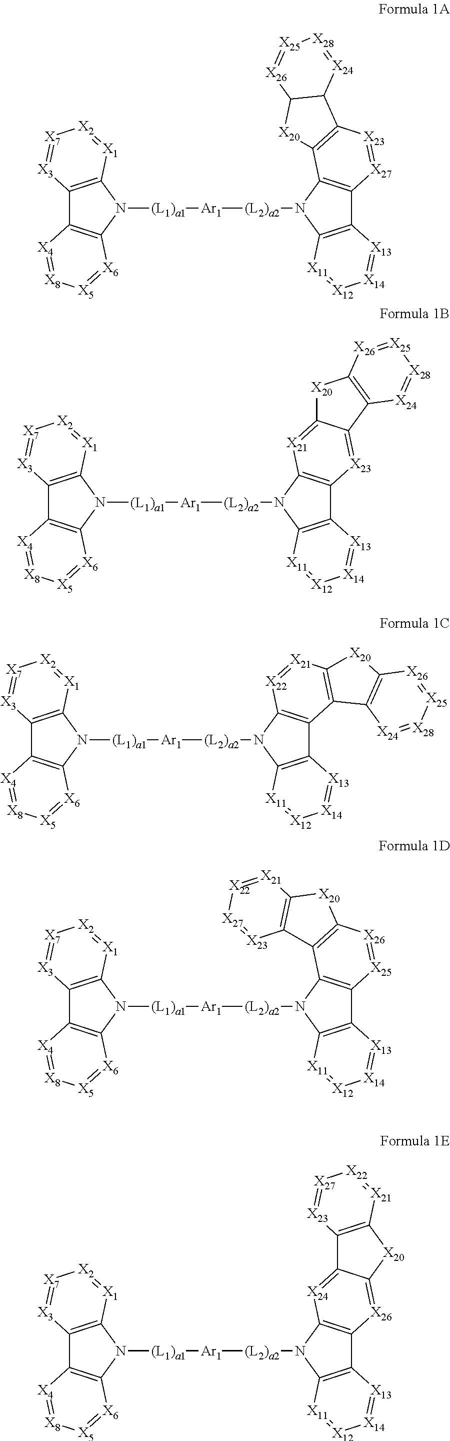

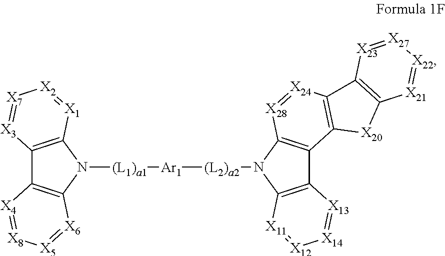

wherein X₁ to X₈, X₁₁ to X₁₄, X₂₀ to X₂₈, Ar₁, L₁, L₂, a1, and a2 in Formulae 1A to 1F are the same as described above.

In one or more embodiments, at least one selected from X₇, X₈, X₁₄, X₂₁, X₂₆, X₂₇, and X₂₈ in Formulae 1A to 1F may be C(CN).

In one or more embodiments, at least two selected from X₇, X₈, X₁₄, X₂₁, X₂₆, X₂₇, and X₂₈ in Formulae 1A to 1F may be C(CN).

In one or more embodiments, at least one selected from X₇ and X₈ in Formulae 1A to 1F may be C(CN).

In one or more embodiments, the condensed cyclic compound represented by Formula 1 may be represented by one selected from Formulae 1A(1), 1A(2), 1B(1), 1B(2), 1C(1), 1C(2), 1D(1), 1D(2), 1E(1), 1E(2), 1F(1), and 1F(2), but is not limited thereto:

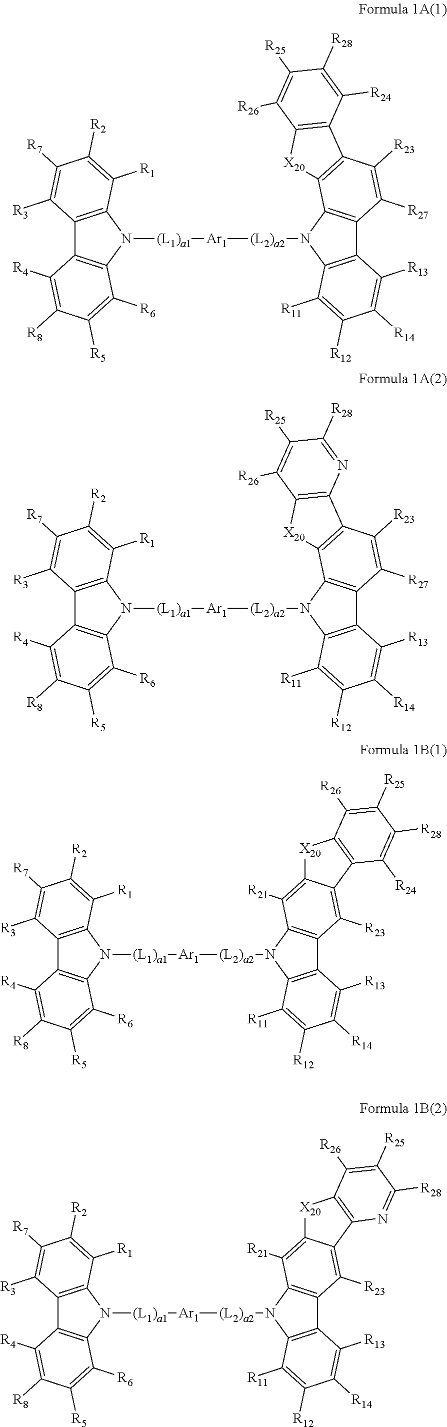

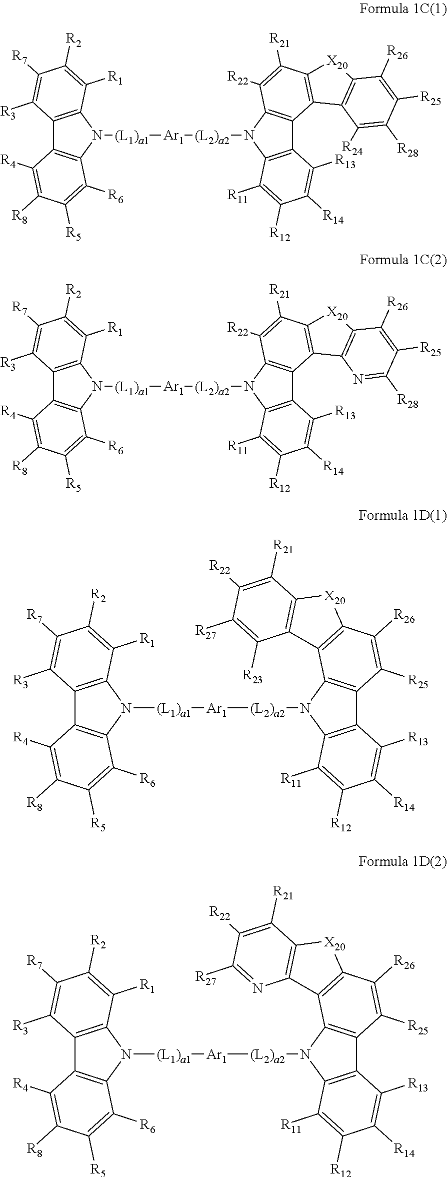

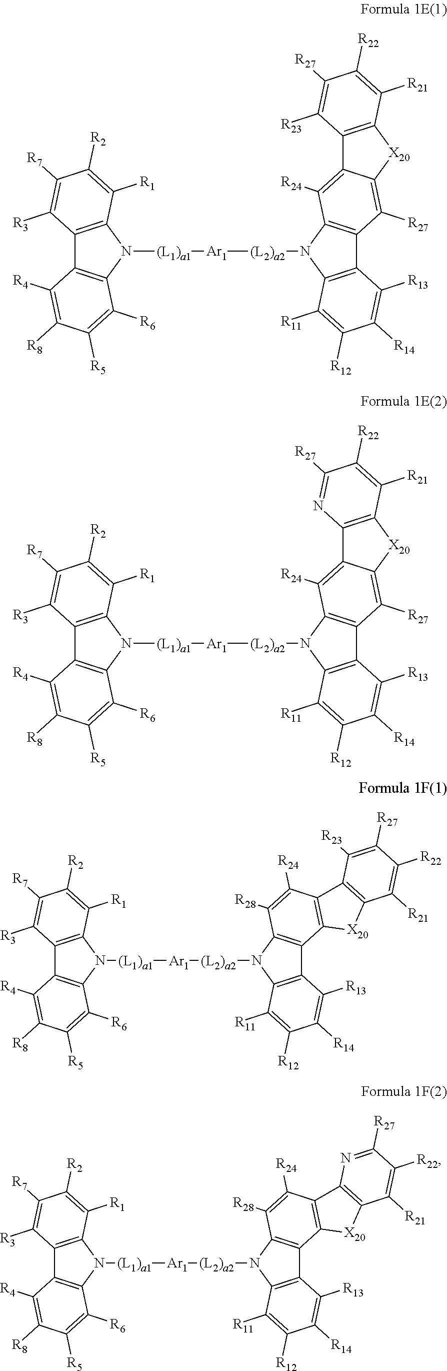

wherein, in Formulae 1A(1), 1A(2), 1B(1), 1B(2), 1C(1), 1C(2), 1D(1), 1D(2), 1E(1), 1E(2), 1F(1), and 1F(2),

Ar₁, L₁, L₂, a1, a2, and X₂₀ are the same as described above,

R₁ to R₈, R₁₁ to R₁₄, and R₂₀ to R₂₉ may each independently be selected from hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a methyl group, an ethyl group, an n-propyl group, an iso-propyl group, an n-butyl group, an iso-butyl group, a sec-butyl group, a tert-butyl group, an n-pentyl group, an iso-pentyl group, a sec-pentyl group, a tert-pentyl group, an n-hexyl group, an iso-hexyl group, a sec-hexyl group, a tert-hexyl group, an n-heptyl group, an iso-heptyl group, a sec-heptyl group, a tert-heptyl group, an n-octyl group, an iso-octyl group, a sec-octyl group, a tert-octyl group, an n-nonyl group, an iso-nonyl group, a sec-nonyl group, a tert-nonyl group, an n-decyl group, an iso-decyl group, a sec-decyl group, a tert-decyl group, a methoxy group, an ethoxy group, a propoxy group, a butoxy group, a pentoxy group, a phenyl group, a naphthyl group, a pyridinyl group, a pyrimidinyl group, a triazinyl group, and —Si(Q₁)(Q₂)(Q₃), and

at least one selected from R₇, R₈, R₁₄, R₂₇, and R₂₈ may be a cyano group.

In one or more embodiments, at least one selected from R₇ and R₈ in Formulae 1A(1), 1A(2), 1B(1), 1B(2), 1C(1), 1C(2), 1D(1), 1D(2), 1E(1), 1E(2), 1F(1), and 1F(2) may be a cyano group.

In one or more embodiments, in Formulae 1A(1), 1A(2), 1B(1), 1B(2), 1C(1), 1C(2), 1D(1), 1D(2), 1E(1), 1E(2), 1F(1), and 1F(2), at least one selected from R₇, R₈, R₁₄, R₂₇, and R₂₈ may be a cyano group, and R₁ to R₆, R₁₁ to R₁₃, R₂₀, and R₂₉ may not be a cyano group.

The condensed cyclic compound may be selected from Compounds 1 to 876, but is not limited thereto:

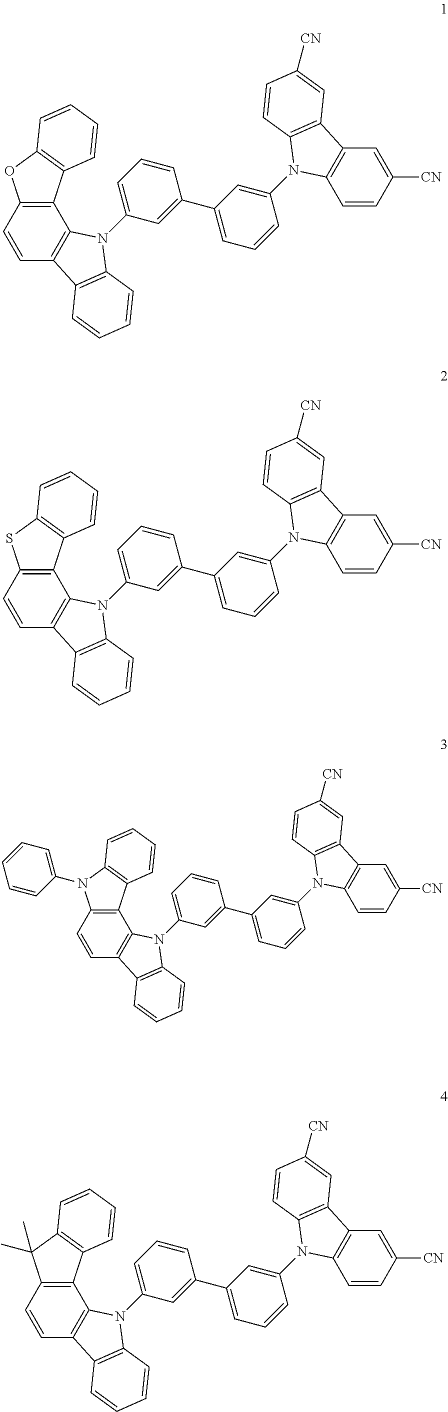 [SMILES: N#Cc1ccc2c(c1)c1cc(C#N)ccc1n2-c1cccc(-c2cccc(-n3c4ccccc4c4ccc5oc6ccccc6c5c43)c2)c1 ; N#Cc1ccc2c(c1)c1cc(C#N)ccc1n2-c1cccc(-c2cccc(-n3c4ccccc4c4ccc5sc6ccccc6c5c43)c2)c1 ; N#Cc1ccc2c(c1)c1cc(C#N)ccc1n2-c1cccc(-c2cccc(-n3c4ccccc4c4ccc5c(c6ccccc6n5-c5ccccc5)c43)c2)c1 ; CC1(C)c2ccccc2-c2c1ccc1c3ccccc3n(-c3cccc(-c4cccc(-n5c6ccc(C#N)cc6c6cc(C#N)ccc65)c4)c3)c21]

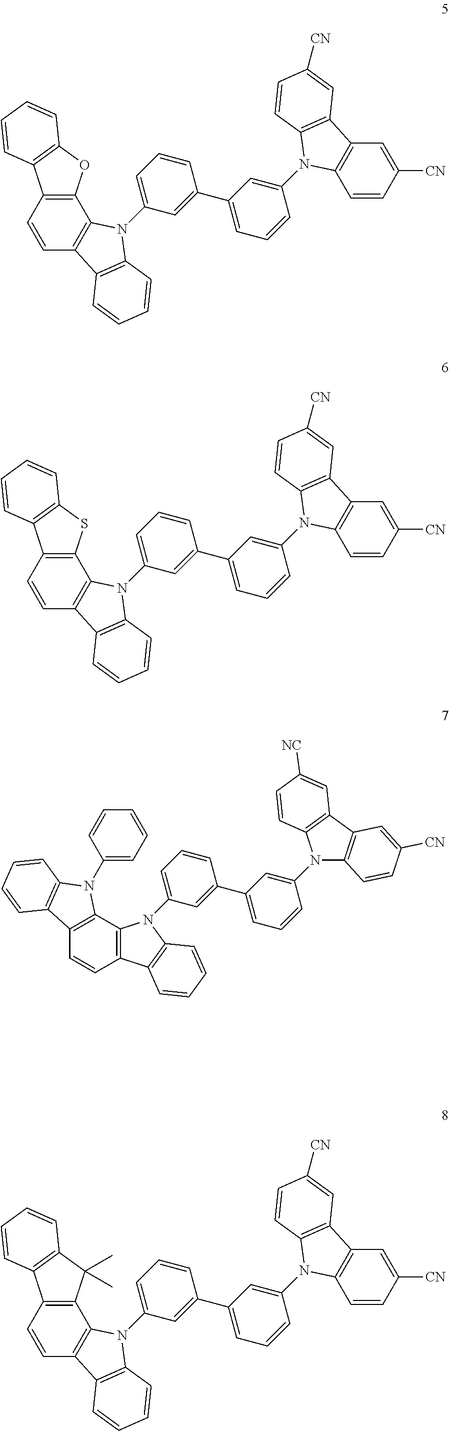 [SMILES: N#Cc1ccc2c(c1)c1cc(C#N)ccc1n2-c1cccc(-c2cccc(-n3c4ccccc4c4ccc5c6ccccc6oc5c43)c2)c1 ; N#Cc1ccc2c(c1)c1cc(C#N)ccc1n2-c1cccc(-c2cccc(-n3c4ccccc4c4ccc5c6ccccc6sc5c43)c2)c1 ; N#Cc1ccc2c(c1)c1cc(C#N)ccc1n2-c1cccc(-c2cccc(-n3c4ccccc4c4ccc5c6ccccc6n(-c6ccccc6)c5c43)c2)c1 ; CC1(C)c2ccccc2-c2ccc3c4ccccc4n(-c4cccc(-c5cccc(-n6c7ccc(C#N)cc7c7cc(C#N)ccc76)c5)c4)c3c21]

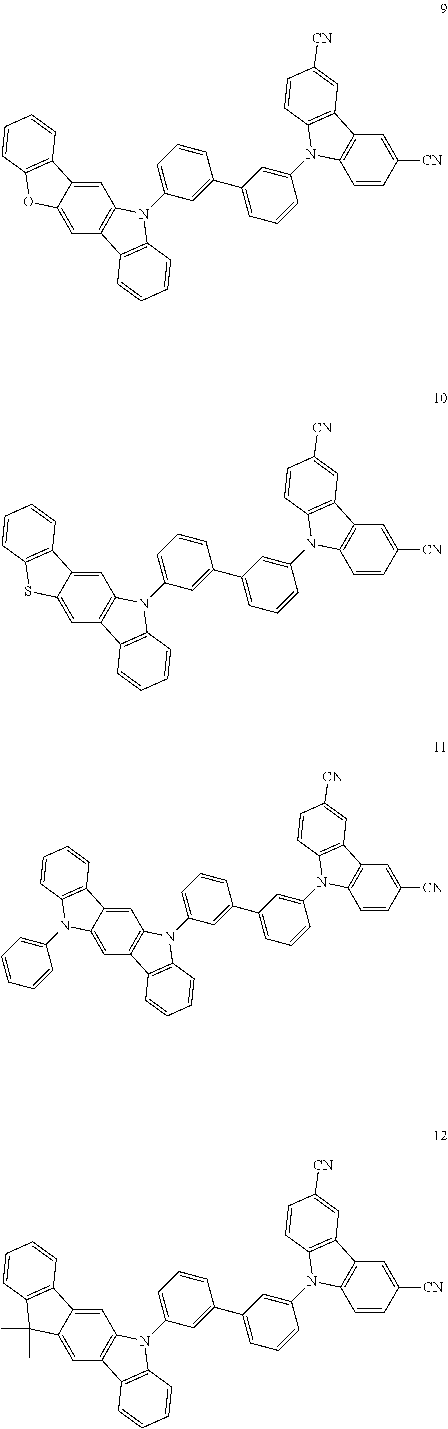 [SMILES: N#Cc1ccc2c(c1)c1cc(C#N)ccc1n2-c1cccc(-c2cccc(-n3c4ccccc4c4cc5oc6ccccc6c5cc43)c2)c1 ; N#Cc1ccc2c(c1)c1cc(C#N)ccc1n2-c1cccc(-c2cccc(-n3c4ccccc4c4cc5sc6ccccc6c5cc43)c2)c1 ; N#Cc1ccc2c(c1)c1cc(C#N)ccc1n2-c1cccc(-c2cccc(-n3c4ccccc4c4cc5c(cc43)c3ccccc3n5-c3ccccc3)c2)c1 ; CC1(C)c2ccccc2-c2cc3c(cc21)c1ccccc1n3-c1cccc(-c2cccc(-n3c4ccc(C#N)cc4c4cc(C#N)ccc43)c2)c1]

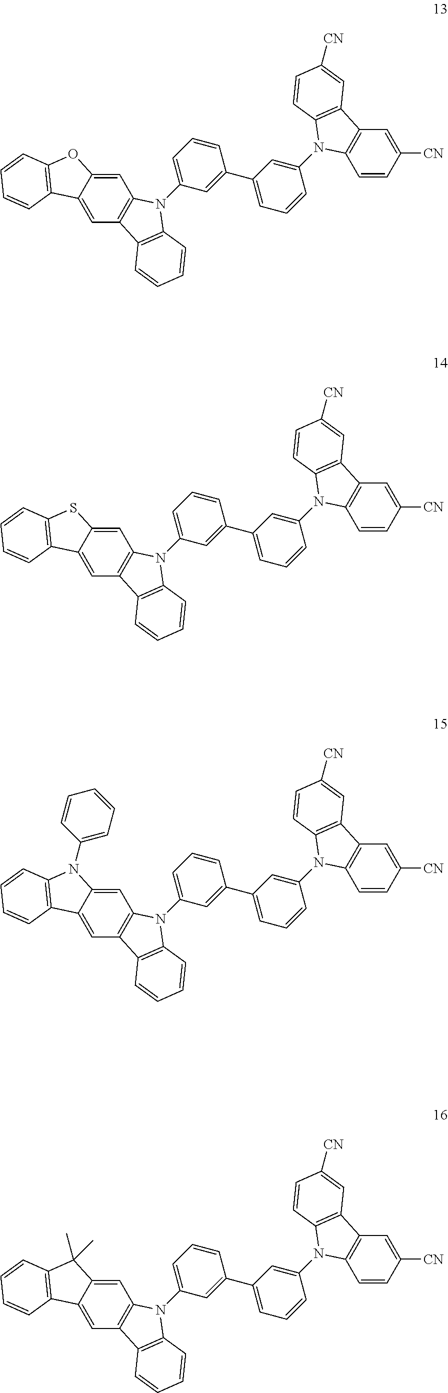 [SMILES: N#Cc1ccc2c(c1)c1cc(C#N)ccc1n2-c1cccc(-c2cccc(-n3c4ccccc4c4cc5c(cc43)oc3ccccc35)c2)c1 ; N#Cc1ccc2c(c1)c1cc(C#N)ccc1n2-c1cccc(-c2cccc(-n3c4ccccc4c4cc5c(cc43)sc3ccccc35)c2)c1 ; N#Cc1ccc2c(c1)c1cc(C#N)ccc1n2-c1cccc(-c2cccc(-n3c4ccccc4c4cc5c6ccccc6n(-c6ccccc6)c5cc43)c2)c1 ; CC1(C)c2ccccc2-c2cc3c4ccccc4n(-c4cccc(-c5cccc(-n6c7ccc(C#N)cc7c7cc(C#N)ccc76)c5)c4)c3cc21]

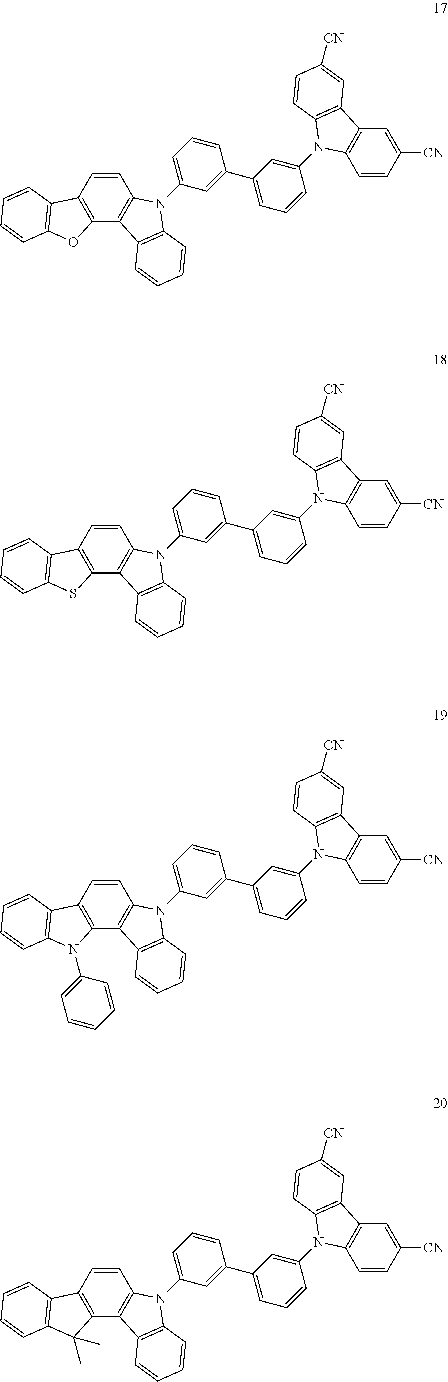 [SMILES: N#Cc1ccc2c(c1)c1cc(C#N)ccc1n2-c1cccc(-c2cccc(-n3c4ccccc4c4c5oc6ccccc6c5ccc43)c2)c1 ; N#Cc1ccc2c(c1)c1cc(C#N)ccc1n2-c1cccc(-c2cccc(-n3c4ccccc4c4c5sc6ccccc6c5ccc43)c2)c1 ; N#Cc1ccc2c(c1)c1cc(C#N)ccc1n2-c1cccc(-c2cccc(-n3c4ccccc4c4c3ccc3c5ccccc5n(-c5ccccc5)c34)c2)c1 ; CC1(C)c2ccccc2-c2ccc3c(c21)c1ccccc1n3-c1cccc(-c2cccc(-n3c4ccc(C#N)cc4c4cc(C#N)ccc43)c2)c1]

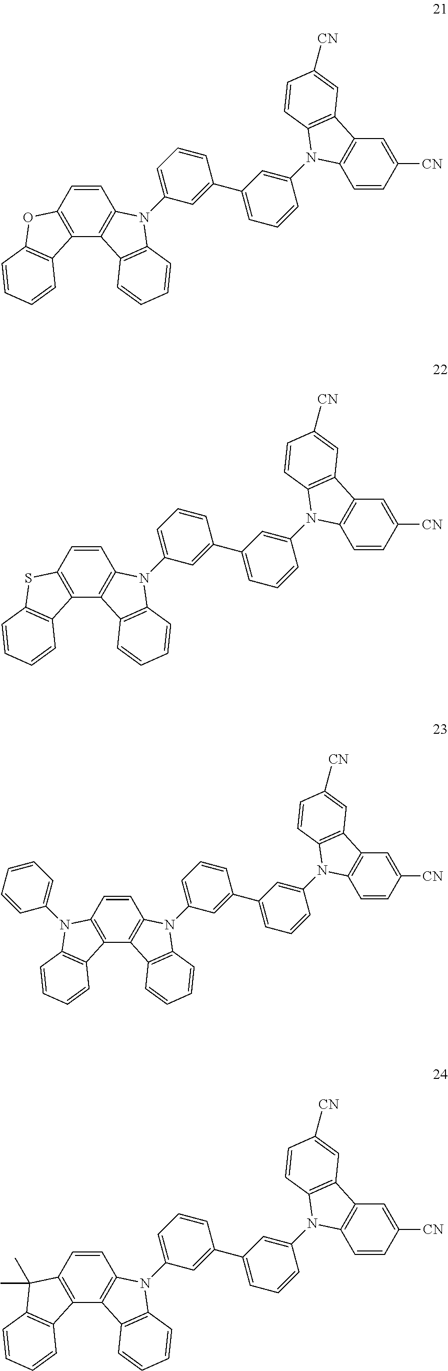 [SMILES: N#Cc1ccc2c(c1)c1cc(C#N)ccc1n2-c1cccc(-c2cccc(-n3c4ccccc4c4c5c(ccc43)oc3ccccc35)c2)c1 ; N#Cc1ccc2c(c1)c1cc(C#N)ccc1n2-c1cccc(-c2cccc(-n3c4ccccc4c4c5c(ccc43)sc3ccccc35)c2)c1 ; N#Cc1ccc2c(c1)c1cc(C#N)ccc1n2-c1cccc(-c2cccc(-n3c4ccccc4c4c5c6ccccc6n(-c6ccccc6)c5ccc43)c2)c1 ; CC1(C)c2ccccc2-c2c1ccc1c2c2ccccc2n1-c1cccc(-c2cccc(-n3c4ccc(C#N)cc4c4cc(C#N)ccc43)c2)c1]

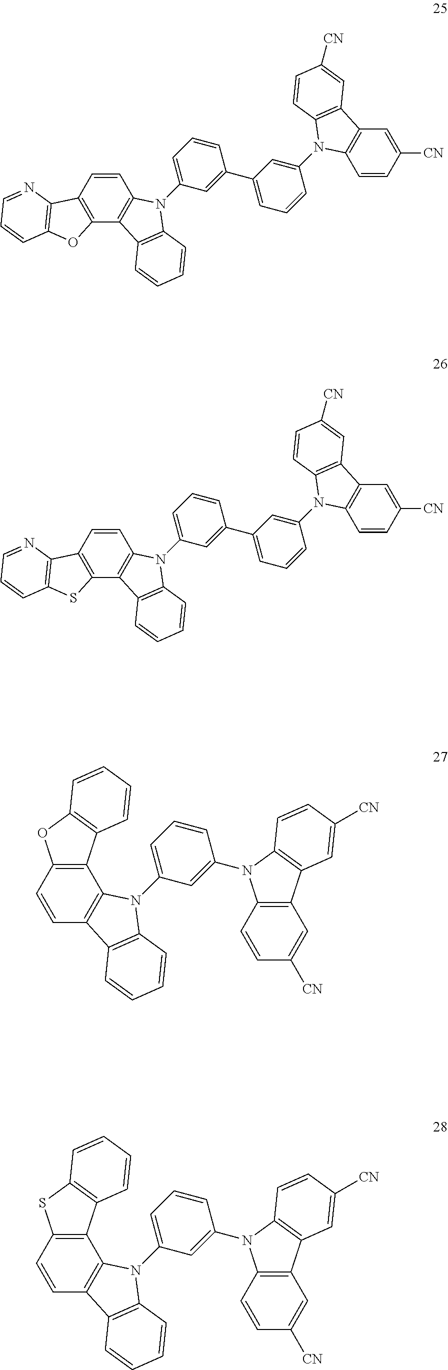 [SMILES: N#Cc1ccc2c(c1)c1cc(C#N)ccc1n2-c1cccc(-c2cccc(-n3c4ccccc4c4c5oc6cccnc6c5ccc43)c2)c1 ; N#Cc1ccc2c(c1)c1cc(C#N)ccc1n2-c1cccc(-c2cccc(-n3c4ccccc4c4c5sc6cccnc6c5ccc43)c2)c1 ; N#Cc1ccc2c(c1)c1cc(C#N)ccc1n2-c1cccc(-n2c3ccccc3c3ccc4oc5ccccc5c4c32)c1 ; N#Cc1ccc2c(c1)c1cc(C#N)ccc1n2-c1cccc(-n2c3ccccc3c3ccc4sc5ccccc5c4c32)c1]

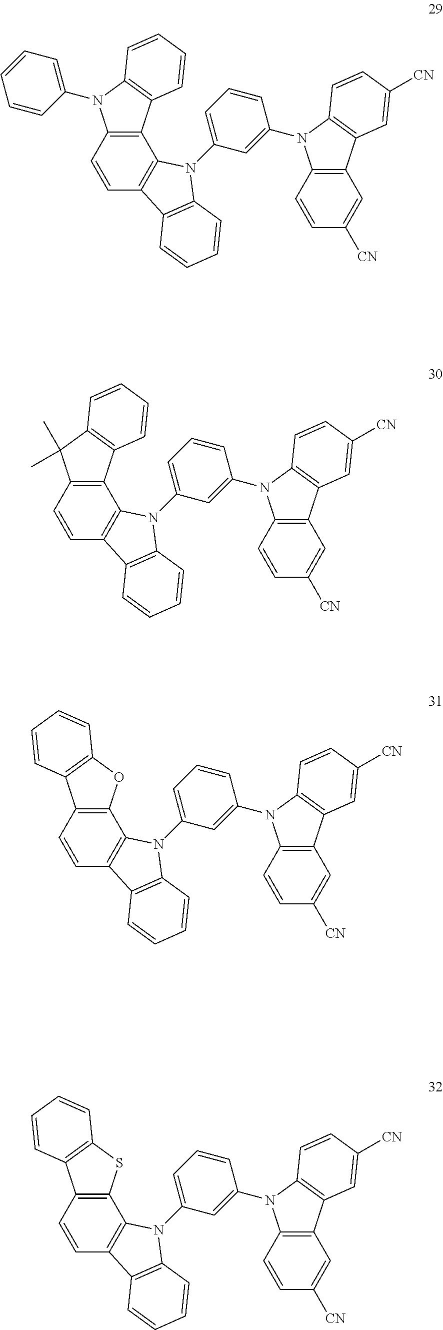 [SMILES: N#Cc1ccc2c(c1)c1cc(C#N)ccc1n2-c1cccc(-n2c3ccccc3c3ccc4c(c5ccccc5n4-c4ccccc4)c32)c1 ; CC1(C)c2ccccc2-c2c1ccc1c3ccccc3n(-c3cccc(-n4c5ccc(C#N)cc5c5cc(C#N)ccc54)c3)c21 ; N#Cc1ccc2c(c1)c1cc(C#N)ccc1n2-c1cccc(-n2c3ccccc3c3ccc4c5ccccc5oc4c32)c1 ; N#Cc1ccc2c(c1)c1cc(C#N)ccc1n2-c1cccc(-n2c3ccccc3c3ccc4c5ccccc5sc4c32)c1]

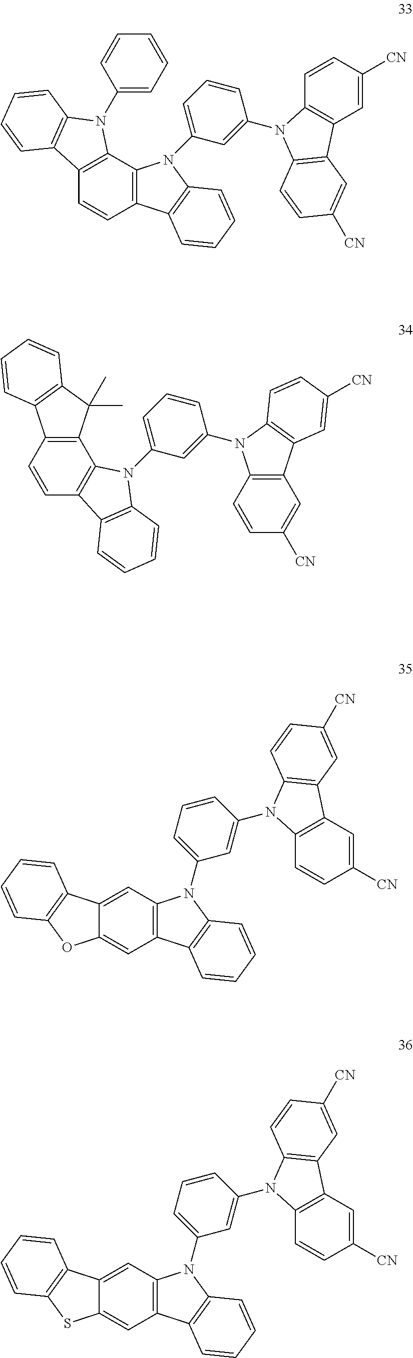 [SMILES: N#Cc1ccc2c(c1)c1cc(C#N)ccc1n2-c1cccc(-n2c3ccccc3c3ccc4c5ccccc5n(-c5ccccc5)c4c32)c1 ; CC1(C)c2ccccc2-c2ccc3c4ccccc4n(-c4cccc(-n5c6ccc(C#N)cc6c6cc(C#N)ccc65)c4)c3c21 ; N#Cc1ccc2c(c1)c1cc(C#N)ccc1n2-c1cccc(-n2c3ccccc3c3cc4oc5ccccc5c4cc32)c1 ; N#Cc1ccc2c(c1)c1cc(C#N)ccc1n2-c1cccc(-n2c3ccccc3c3cc4sc5ccccc5c4cc32)c1]

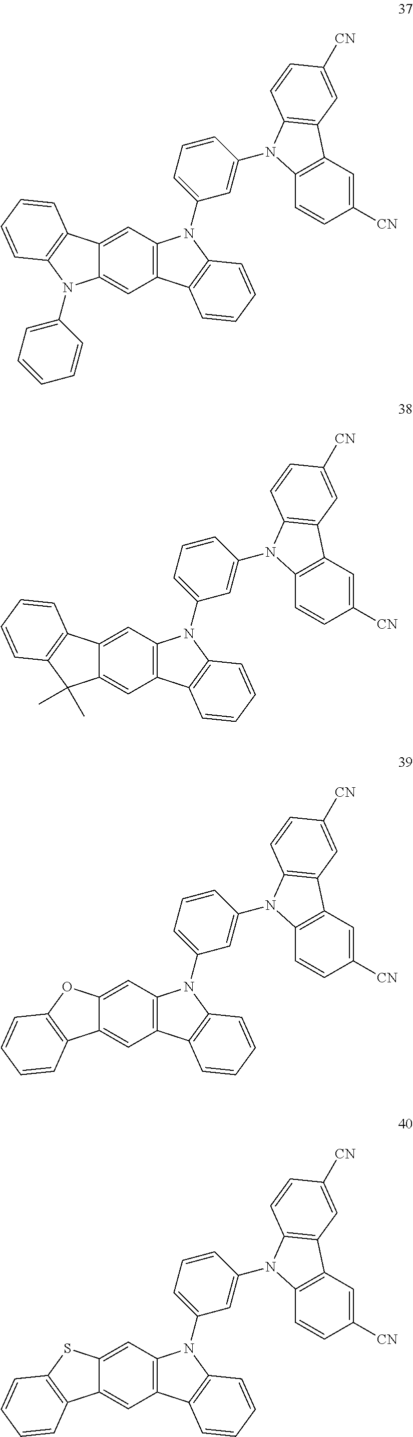 [SMILES: N#Cc1ccc2c(c1)c1cc(C#N)ccc1n2-c1cccc(-n2c3ccccc3c3cc4c(cc32)c2ccccc2n4-c2ccccc2)c1 ; CC1(C)c2ccccc2-c2cc3c(cc21)c1ccccc1n3-c1cccc(-n2c3ccc(C#N)cc3c3cc(C#N)ccc32)c1 ; N#Cc1ccc2c(c1)c1cc(C#N)ccc1n2-c1cccc(-n2c3ccccc3c3cc4c(cc32)oc2ccccc24)c1 ; N#Cc1ccc2c(c1)c1cc(C#N)ccc1n2-c1cccc(-n2c3ccccc3c3cc4c(cc32)sc2ccccc24)c1]

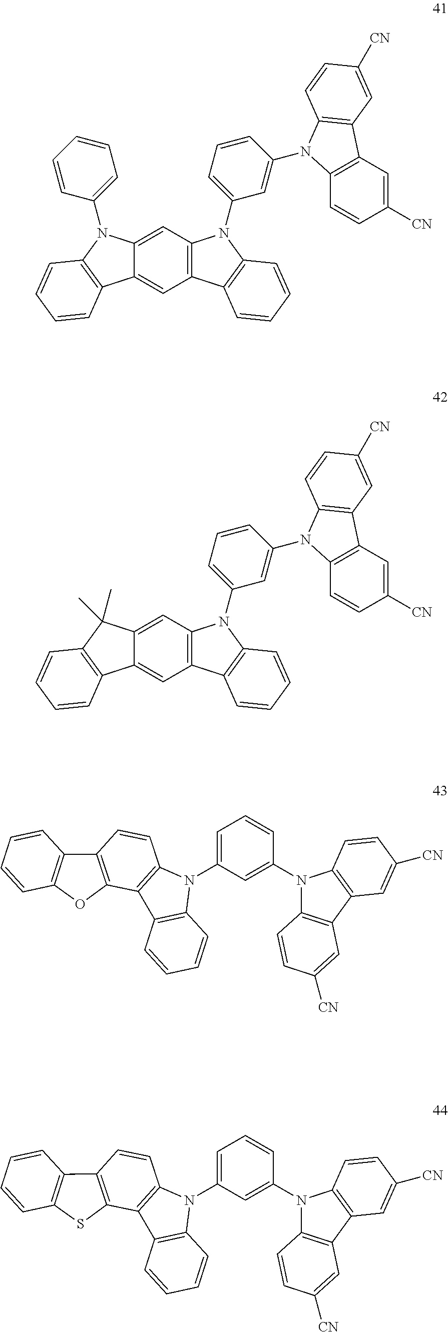 [SMILES: N#Cc1ccc2c(c1)c1cc(C#N)ccc1n2-c1cccc(-n2c3ccccc3c3cc4c5ccccc5n(-c5ccccc5)c4cc32)c1 ; CC1(C)c2ccccc2-c2cc3c4ccccc4n(-c4cccc(-n5c6ccc(C#N)cc6c6cc(C#N)ccc65)c4)c3cc21 ; N#Cc1ccc2c(c1)c1cc(C#N)ccc1n2-c1cccc(-n2c3ccccc3c3c4oc5ccccc5c4ccc32)c1 ; N#Cc1ccc2c(c1)c1cc(C#N)ccc1n2-c1cccc(-n2c3ccccc3c3c4sc5ccccc5c4ccc32)c1]

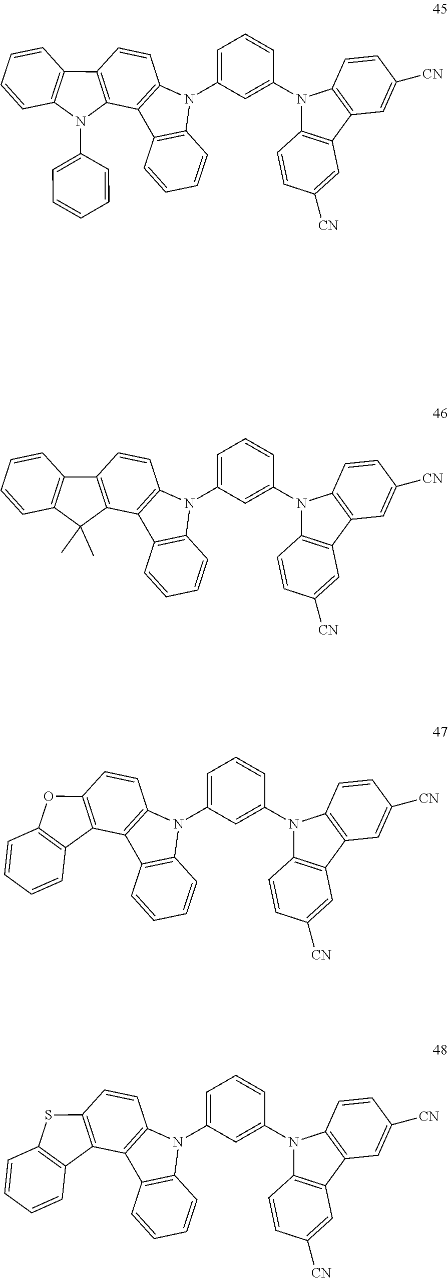 [SMILES: N#Cc1ccc2c(c1)c1cc(C#N)ccc1n2-c1cccc(-n2c3ccccc3c3c2ccc2c4ccccc4n(-c4ccccc4)c23)c1 ; CC1(C)c2ccccc2-c2ccc3c(c21)c1ccccc1n3-c1cccc(-n2c3ccc(C#N)cc3c3cc(C#N)ccc32)c1 ; N#Cc1ccc2c(c1)c1cc(C#N)ccc1n2-c1cccc(-n2c3ccccc3c3c4c(ccc32)oc2ccccc24)c1 ; N#Cc1ccc2c(c1)c1cc(C#N)ccc1n2-c1cccc(-n2c3ccccc3c3c4c(ccc32)sc2ccccc24)c1]

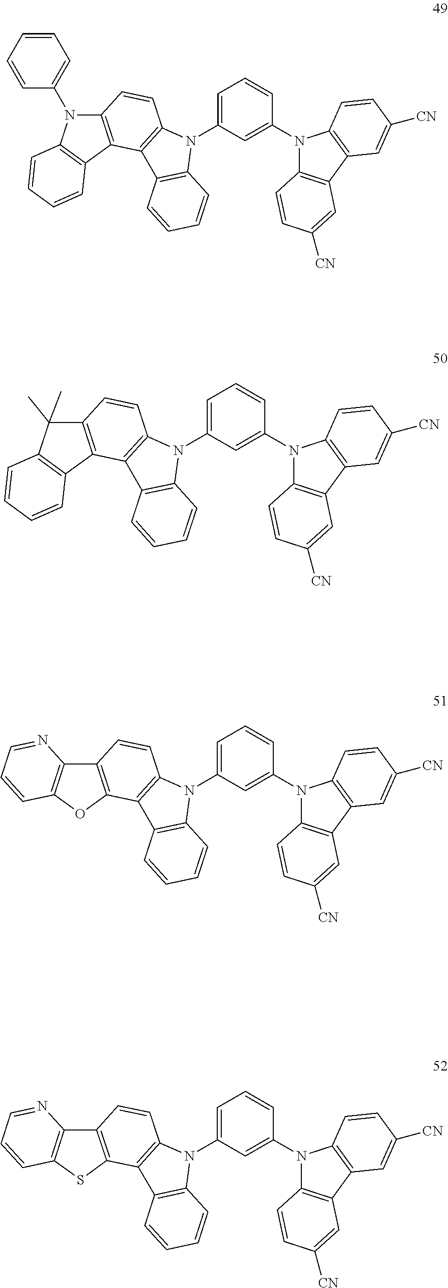 [SMILES: N#Cc1ccc2c(c1)c1cc(C#N)ccc1n2-c1cccc(-n2c3ccccc3c3c4c5ccccc5n(-c5ccccc5)c4ccc32)c1 ; CC1(C)c2ccccc2-c2c1ccc1c2c2ccccc2n1-c1cccc(-n2c3ccc(C#N)cc3c3cc(C#N)ccc32)c1 ; N#Cc1ccc2c(c1)c1cc(C#N)ccc1n2-c1cccc(-n2c3ccccc3c3c4oc5cccnc5c4ccc32)c1 ; N#Cc1ccc2c(c1)c1cc(C#N)ccc1n2-c1cccc(-n2c3ccccc3c3c4sc5cccnc5c4ccc32)c1]

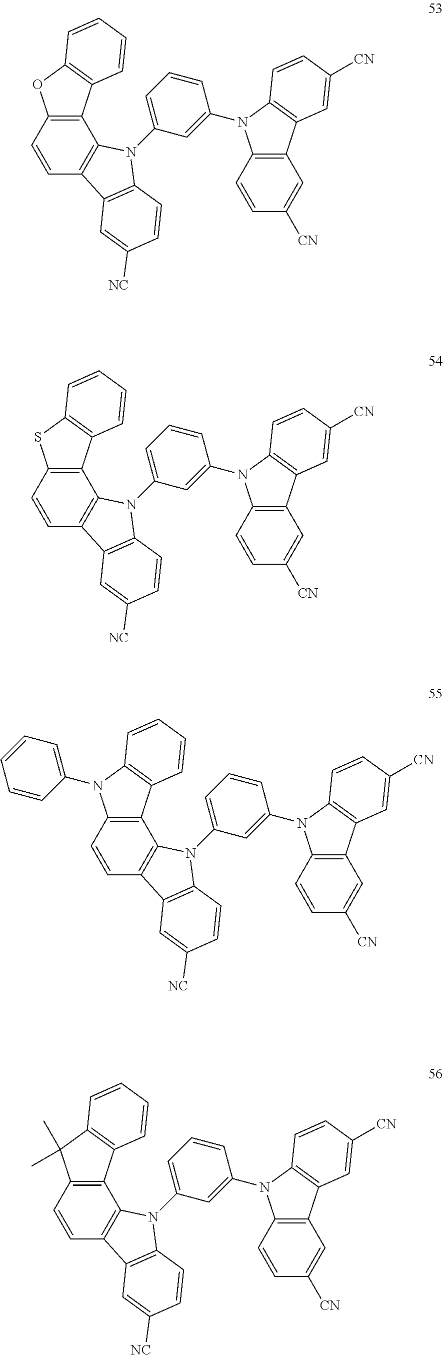 [SMILES: N#Cc1ccc2c(c1)c1cc(C#N)ccc1n2-c1cccc(-n2c3ccc(C#N)cc3c3ccc4oc5ccccc5c4c32)c1 ; N#Cc1ccc2c(c1)c1cc(C#N)ccc1n2-c1cccc(-n2c3ccc(C#N)cc3c3ccc4sc5ccccc5c4c32)c1 ; N#Cc1ccc2c(c1)c1cc(C#N)ccc1n2-c1cccc(-n2c3ccc(C#N)cc3c3ccc4c(c5ccccc5n4-c4ccccc4)c32)c1 ; CC1(C)c2ccccc2-c2c1ccc1c3cc(C#N)ccc3n(-c3cccc(-n4c5ccc(C#N)cc5c5cc(C#N)ccc54)c3)c21]

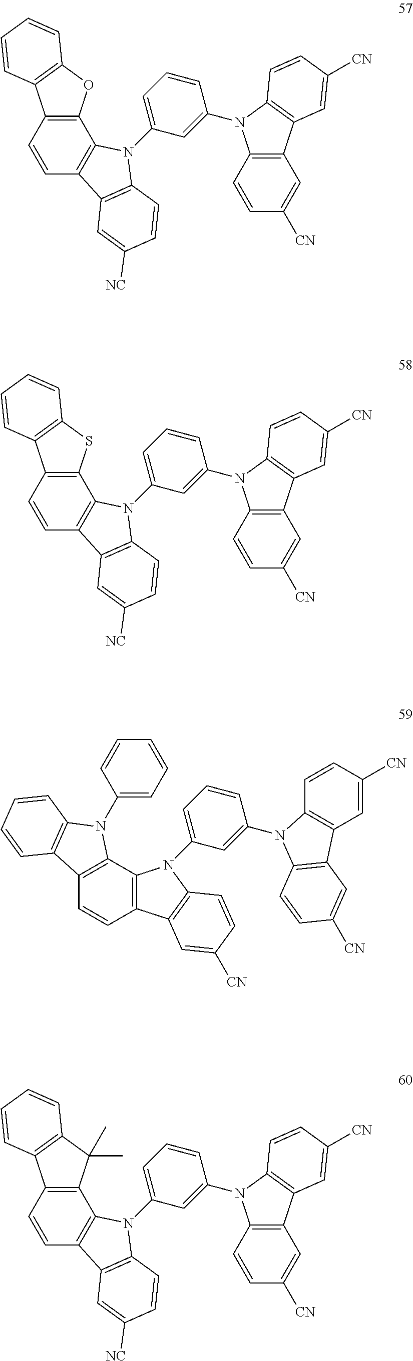 [SMILES: N#Cc1ccc2c(c1)c1cc(C#N)ccc1n2-c1cccc(-n2c3ccc(C#N)cc3c3ccc4c5ccccc5oc4c32)c1 ; N#Cc1ccc2c(c1)c1cc(C#N)ccc1n2-c1cccc(-n2c3ccc(C#N)cc3c3ccc4c5ccccc5sc4c32)c1 ; N#Cc1ccc2c(c1)c1cc(C#N)ccc1n2-c1cccc(-n2c3ccc(C#N)cc3c3ccc4c5ccccc5n(-c5ccccc5)c4c32)c1 ; CC1(C)c2ccccc2-c2ccc3c4cc(C#N)ccc4n(-c4cccc(-n5c6ccc(C#N)cc6c6cc(C#N)ccc65)c4)c3c21]

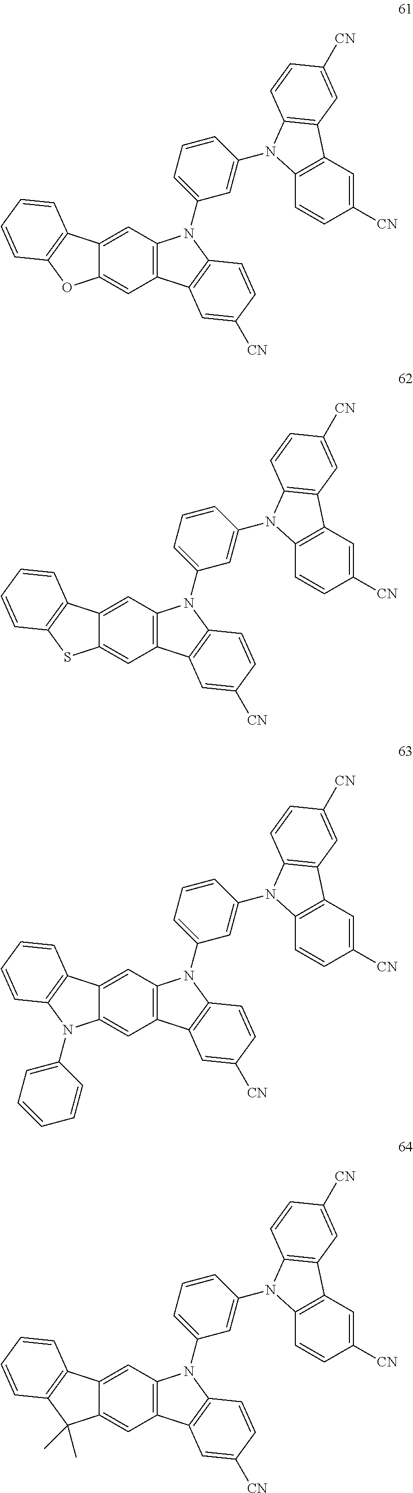 [SMILES: N#Cc1ccc2c(c1)c1cc(C#N)ccc1n2-c1cccc(-n2c3ccc(C#N)cc3c3cc4oc5ccccc5c4cc32)c1 ; N#Cc1ccc2c(c1)c1cc(C#N)ccc1n2-c1cccc(-n2c3ccc(C#N)cc3c3cc4sc5ccccc5c4cc32)c1 ; N#Cc1ccc2c(c1)c1cc(C#N)ccc1n2-c1cccc(-n2c3ccc(C#N)cc3c3cc4c(cc32)c2ccccc2n4-c2ccccc2)c1 ; CC1(C)c2ccccc2-c2cc3c(cc21)c1cc(C#N)ccc1n3-c1cccc(-n2c3ccc(C#N)cc3c3cc(C#N)ccc32)c1]

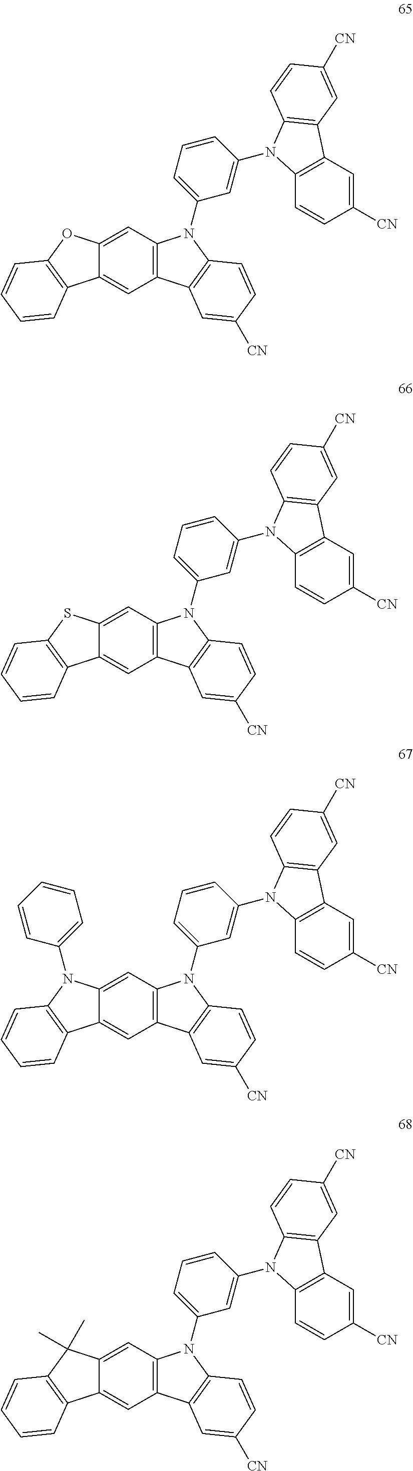 [SMILES: N#Cc1ccc2c(c1)c1cc(C#N)ccc1n2-c1cccc(-n2c3ccc(C#N)cc3c3cc4c(cc32)oc2ccccc24)c1 ; N#Cc1ccc2c(c1)c1cc(C#N)ccc1n2-c1cccc(-n2c3ccc(C#N)cc3c3cc4c(cc32)sc2ccccc24)c1 ; N#Cc1ccc2c(c1)c1cc(C#N)ccc1n2-c1cccc(-n2c3ccc(C#N)cc3c3cc4c5ccccc5n(-c5ccccc5)c4cc32)c1 ; CC1(C)c2ccccc2-c2cc3c4cc(C#N)ccc4n(-c4cccc(-n5c6ccc(C#N)cc6c6cc(C#N)ccc65)c4)c3cc21]

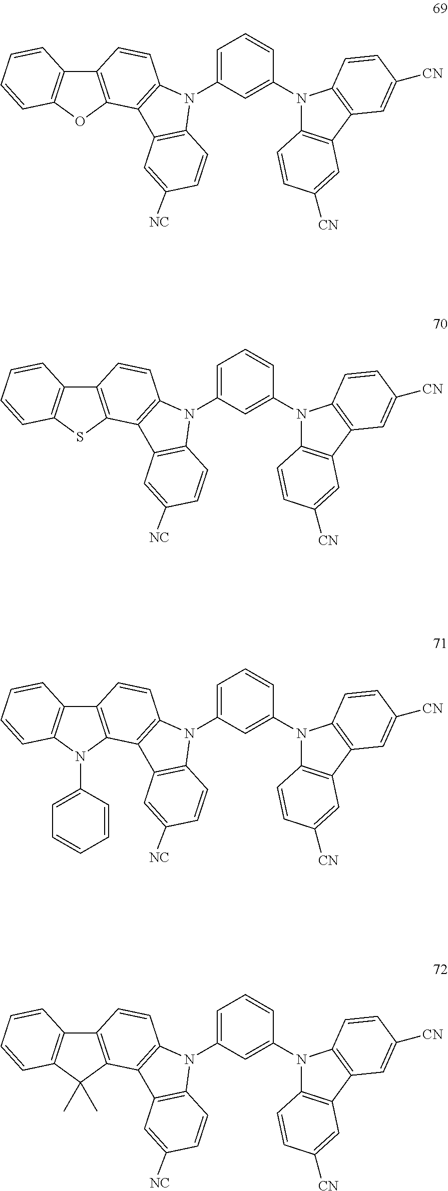

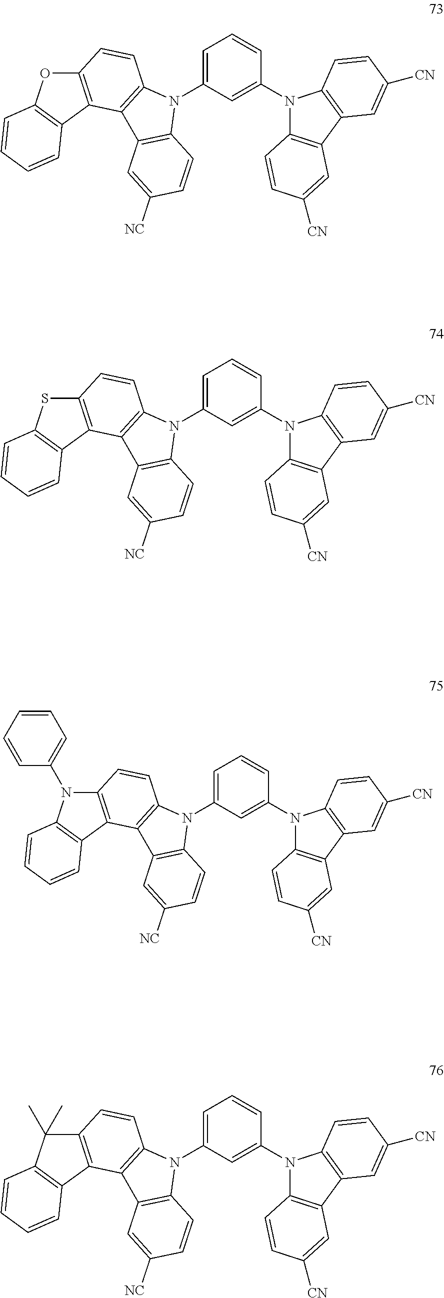 [SMILES: N#Cc1ccc2c(c1)c1cc(C#N)ccc1n2-c1cccc(-n2c3ccc(C#N)cc3c3c4c(ccc32)oc2ccccc24)c1 ; N#Cc1ccc2c(c1)c1cc(C#N)ccc1n2-c1cccc(-n2c3ccc(C#N)cc3c3c4c(ccc32)sc2ccccc24)c1 ; N#Cc1ccc2c(c1)c1cc(C#N)ccc1n2-c1cccc(-n2c3ccc(C#N)cc3c3c4c5ccccc5n(-c5ccccc5)c4ccc32)c1 ; CC1(C)c2ccccc2-c2c1ccc1c2c2cc(C#N)ccc2n1-c1cccc(-n2c3ccc(C#N)cc3c3cc(C#N)ccc32)c1]

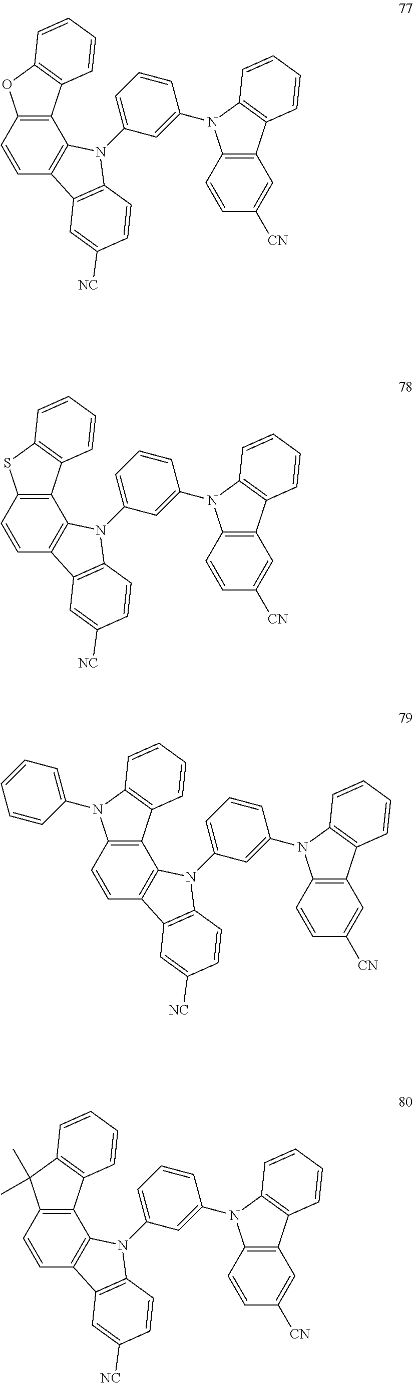 [SMILES: N#Cc1ccc2c(c1)c1ccccc1n2-c1cccc(-n2c3ccc(C#N)cc3c3ccc4oc5ccccc5c4c32)c1 ; N#Cc1ccc2c(c1)c1ccccc1n2-c1cccc(-n2c3ccc(C#N)cc3c3ccc4sc5ccccc5c4c32)c1 ; N#Cc1ccc2c(c1)c1ccccc1n2-c1cccc(-n2c3ccc(C#N)cc3c3ccc4c(c5ccccc5n4-c4ccccc4)c32)c1 ; CC1(C)c2ccccc2-c2c1ccc1c3cc(C#N)ccc3n(-c3cccc(-n4c5ccccc5c5cc(C#N)ccc54)c3)c21]

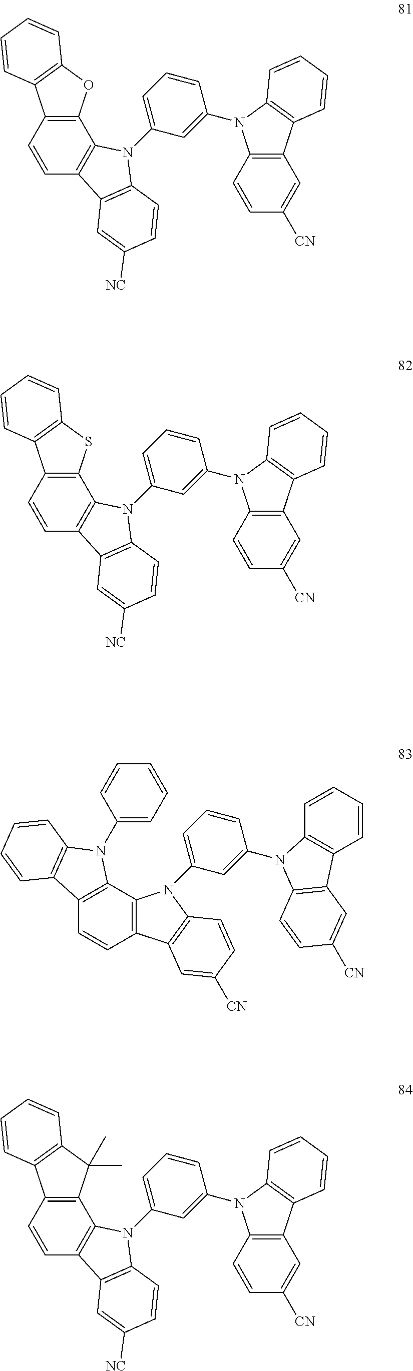 [SMILES: N#Cc1ccc2c(c1)c1ccccc1n2-c1cccc(-n2c3ccc(C#N)cc3c3ccc4c5ccccc5oc4c32)c1 ; N#Cc1ccc2c(c1)c1ccccc1n2-c1cccc(-n2c3ccc(C#N)cc3c3ccc4c5ccccc5sc4c32)c1 ; N#Cc1ccc2c(c1)c1ccccc1n2-c1cccc(-n2c3ccc(C#N)cc3c3ccc4c5ccccc5n(-c5ccccc5)c4c32)c1 ; CC1(C)c2ccccc2-c2ccc3c4cc(C#N)ccc4n(-c4cccc(-n5c6ccccc6c6cc(C#N)ccc65)c4)c3c21]

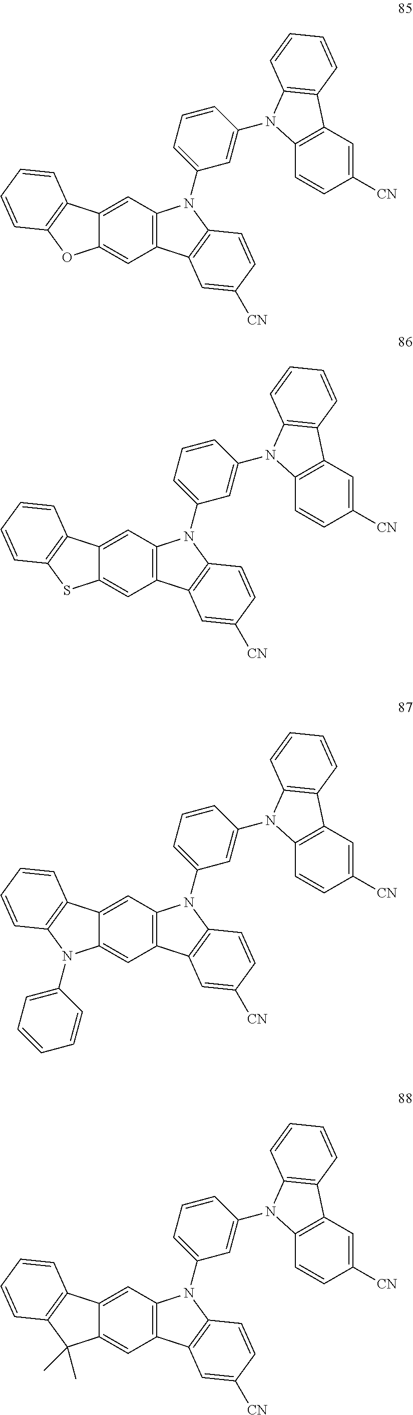 [SMILES: N#Cc1ccc2c(c1)c1ccccc1n2-c1cccc(-n2c3ccc(C#N)cc3c3cc4oc5ccccc5c4cc32)c1 ; N#Cc1ccc2c(c1)c1ccccc1n2-c1cccc(-n2c3ccc(C#N)cc3c3cc4sc5ccccc5c4cc32)c1 ; N#Cc1ccc2c(c1)c1ccccc1n2-c1cccc(-n2c3ccc(C#N)cc3c3cc4c(cc32)c2ccccc2n4-c2ccccc2)c1 ; CC1(C)c2ccccc2-c2cc3c(cc21)c1cc(C#N)ccc1n3-c1cccc(-n2c3ccccc3c3cc(C#N)ccc32)c1]

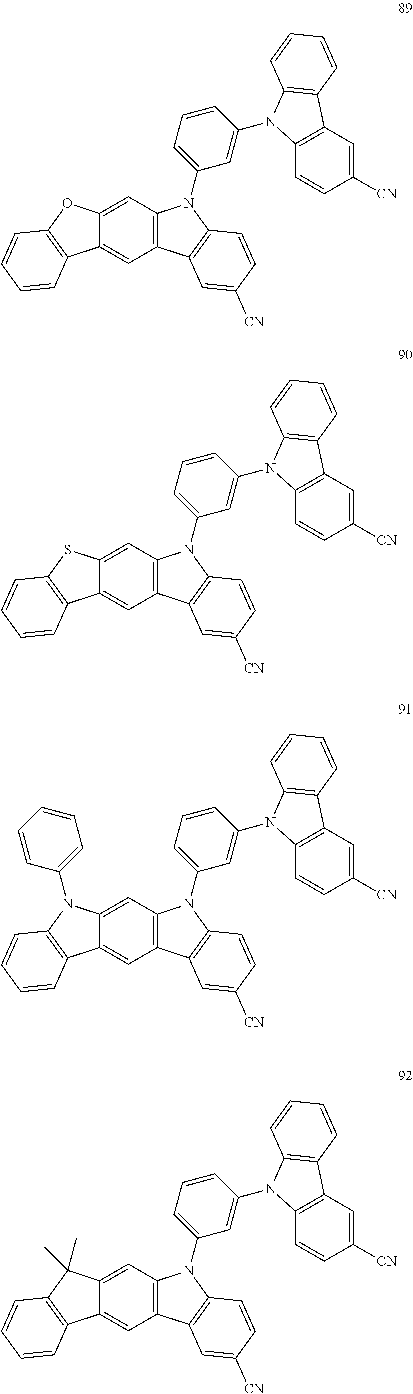 [SMILES: N#Cc1ccc2c(c1)c1ccccc1n2-c1cccc(-n2c3ccc(C#N)cc3c3cc4c(cc32)oc2ccccc24)c1 ; N#Cc1ccc2c(c1)c1ccccc1n2-c1cccc(-n2c3ccc(C#N)cc3c3cc4c(cc32)sc2ccccc24)c1 ; N#Cc1ccc2c(c1)c1ccccc1n2-c1cccc(-n2c3ccc(C#N)cc3c3cc4c5ccccc5n(-c5ccccc5)c4cc32)c1 ; CC1(C)c2ccccc2-c2cc3c4cc(C#N)ccc4n(-c4cccc(-n5c6ccccc6c6cc(C#N)ccc65)c4)c3cc21]

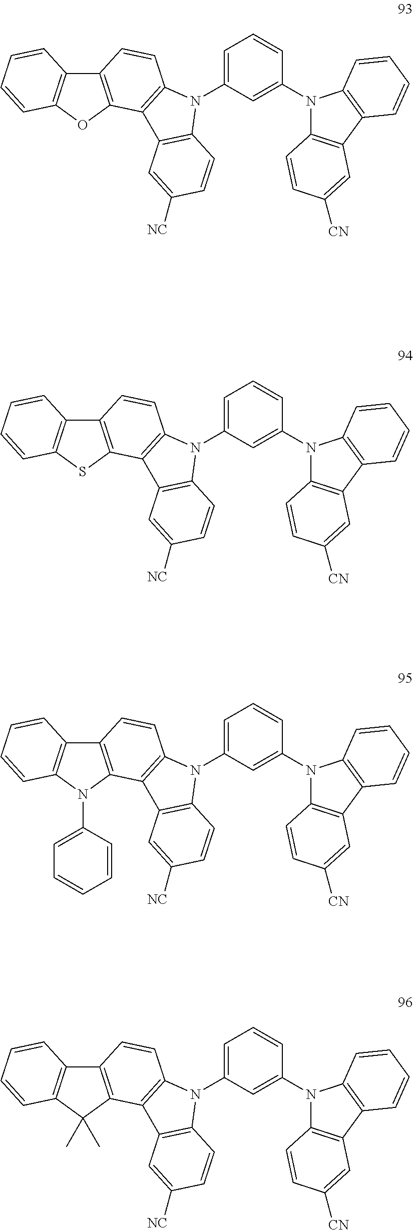 [SMILES: N#Cc1ccc2c(c1)c1ccccc1n2-c1cccc(-n2c3ccc(C#N)cc3c3c4oc5ccccc5c4ccc32)c1 ; N#Cc1ccc2c(c1)c1ccccc1n2-c1cccc(-n2c3ccc(C#N)cc3c3c4sc5ccccc5c4ccc32)c1 ; N#Cc1ccc2c(c1)c1ccccc1n2-c1cccc(-n2c3ccc(C#N)cc3c3c2ccc2c4ccccc4n(-c4ccccc4)c23)c1 ; CC1(C)c2ccccc2-c2ccc3c(c21)c1cc(C#N)ccc1n3-c1cccc(-n2c3ccccc3c3cc(C#N)ccc32)c1]

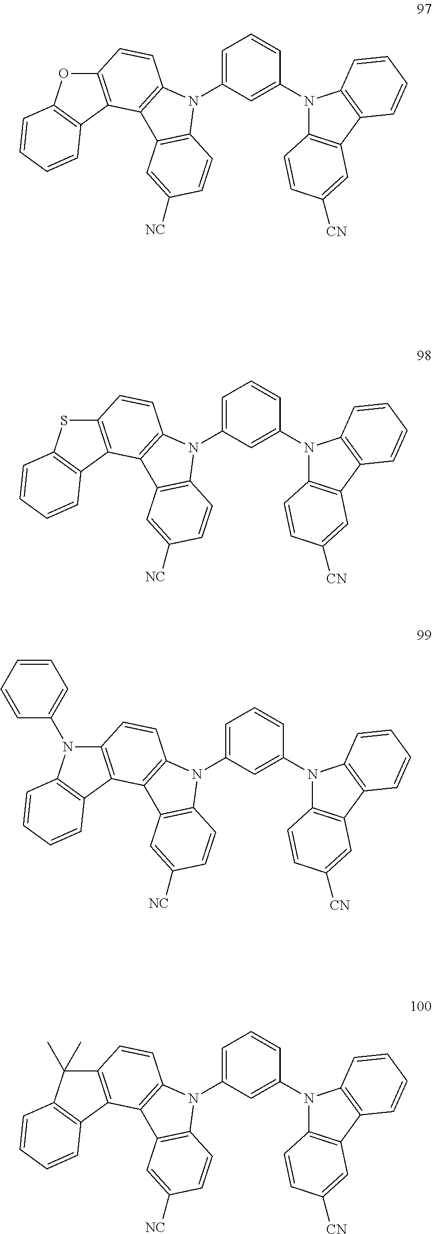 [SMILES: N#Cc1ccc2c(c1)c1ccccc1n2-c1cccc(-n2c3ccc(C#N)cc3c3c4c(ccc32)oc2ccccc24)c1 ; N#Cc1ccc2c(c1)c1ccccc1n2-c1cccc(-n2c3ccc(C#N)cc3c3c4c(ccc32)sc2ccccc24)c1 ; N#Cc1ccc2c(c1)c1ccccc1n2-c1cccc(-n2c3ccc(C#N)cc3c3c4c5ccccc5n(-c5ccccc5)c4ccc32)c1 ; CC1(C)c2ccccc2-c2c1ccc1c2c2cc(C#N)ccc2n1-c1cccc(-n2c3ccccc3c3cc(C#N)ccc32)c1]

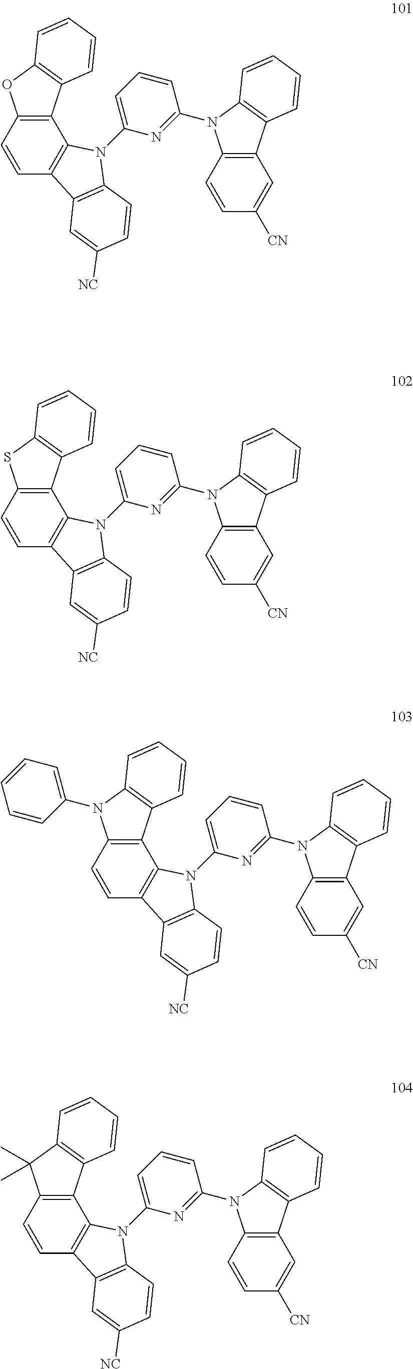 [SMILES: N#Cc1ccc2c(c1)c1ccccc1n2-c1cccc(-n2c3ccc(C#N)cc3c3ccc4oc5ccccc5c4c32)n1 ; N#Cc1ccc2c(c1)c1ccccc1n2-c1cccc(-n2c3ccc(C#N)cc3c3ccc4sc5ccccc5c4c32)n1 ; N#Cc1ccc2c(c1)c1ccccc1n2-c1cccc(-n2c3ccc(C#N)cc3c3ccc4c(c5ccccc5n4-c4ccccc4)c32)n1 ; CC1(C)c2ccccc2-c2c1ccc1c3cc(C#N)ccc3n(-c3cccc(-n4c5ccccc5c5cc(C#N)ccc54)n3)c21]

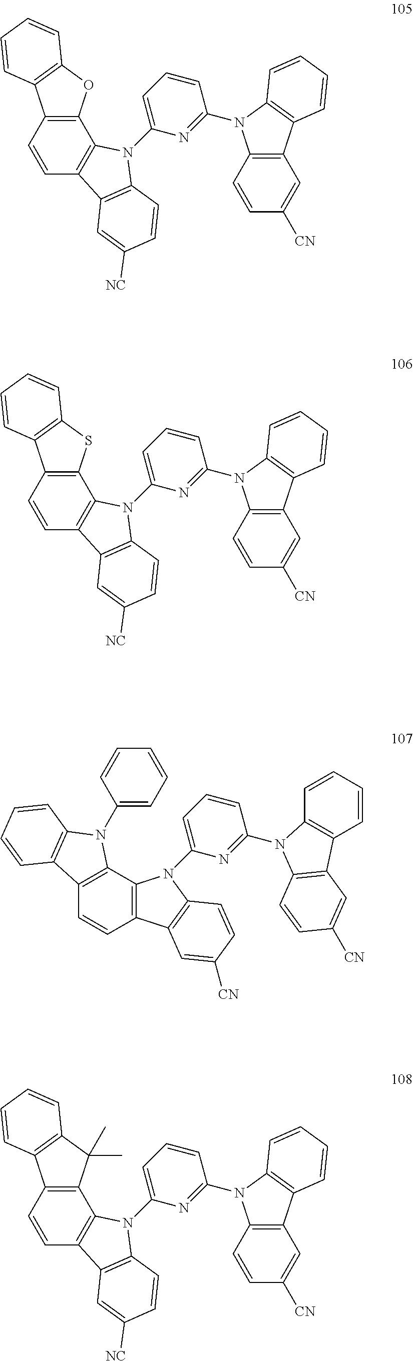 [SMILES: N#Cc1ccc2c(c1)c1ccccc1n2-c1cccc(-n2c3ccc(C#N)cc3c3ccc4c5ccccc5oc4c32)n1 ; N#Cc1ccc2c(c1)c1ccccc1n2-c1cccc(-n2c3ccc(C#N)cc3c3ccc4c5ccccc5sc4c32)n1 ; N#Cc1ccc2c(c1)c1ccccc1n2-c1cccc(-n2c3ccc(C#N)cc3c3ccc4c5ccccc5n(-c5ccccc5)c4c32)n1 ; CC1(C)c2ccccc2-c2ccc3c4cc(C#N)ccc4n(-c4cccc(-n5c6ccccc6c6cc(C#N)ccc65)n4)c3c21]

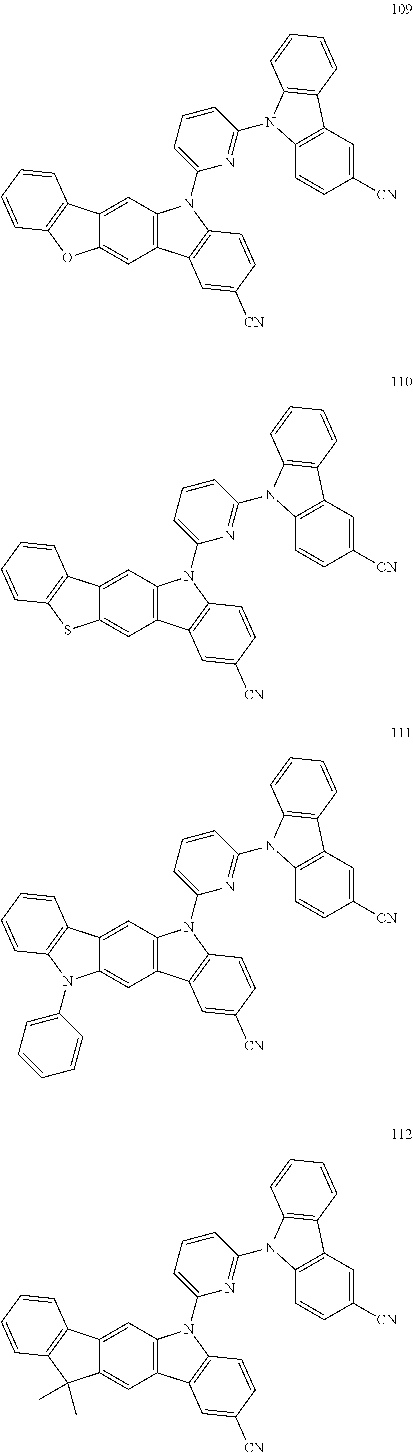 [SMILES: N#Cc1ccc2c(c1)c1ccccc1n2-c1cccc(-n2c3ccc(C#N)cc3c3cc4oc5ccccc5c4cc32)n1 ; N#Cc1ccc2c(c1)c1ccccc1n2-c1cccc(-n2c3ccc(C#N)cc3c3cc4sc5ccccc5c4cc32)n1 ; N#Cc1ccc2c(c1)c1ccccc1n2-c1cccc(-n2c3ccc(C#N)cc3c3cc4c(cc32)c2ccccc2n4-c2ccccc2)n1 ; CC1(C)c2ccccc2-c2cc3c(cc21)c1cc(C#N)ccc1n3-c1cccc(-n2c3ccccc3c3cc(C#N)ccc32)n1]

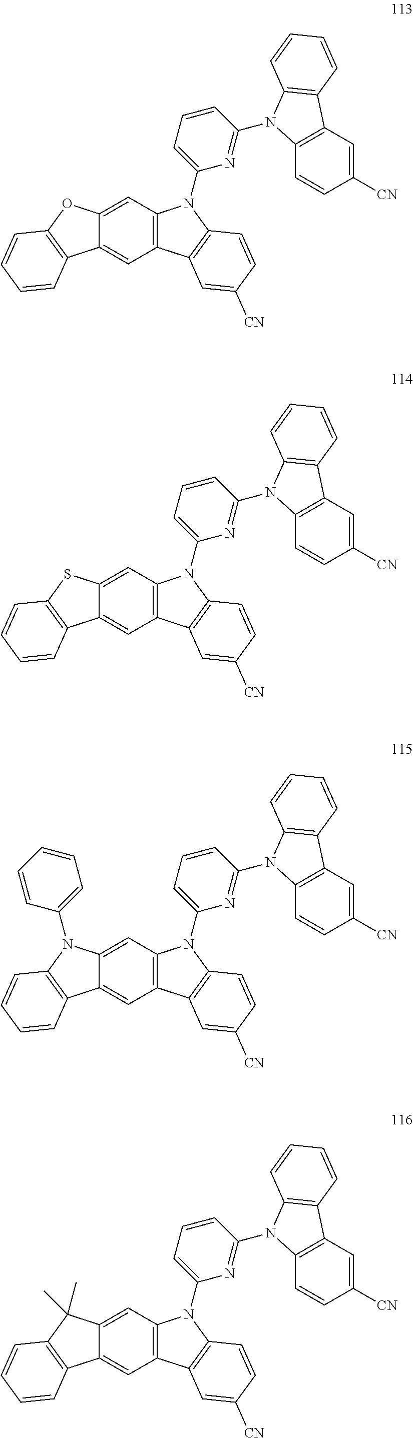 [SMILES: N#Cc1ccc2c(c1)c1ccccc1n2-c1cccc(-n2c3ccc(C#N)cc3c3cc4c(cc32)oc2ccccc24)n1 ; N#Cc1ccc2c(c1)c1ccccc1n2-c1cccc(-n2c3ccc(C#N)cc3c3cc4c(cc32)sc2ccccc24)n1 ; N#Cc1ccc2c(c1)c1ccccc1n2-c1cccc(-n2c3ccc(C#N)cc3c3cc4c5ccccc5n(-c5ccccc5)c4cc32)n1 ; CC1(C)c2ccccc2-c2cc3c4cc(C#N)ccc4n(-c4cccc(-n5c6ccccc6c6cc(C#N)ccc65)n4)c3cc21]

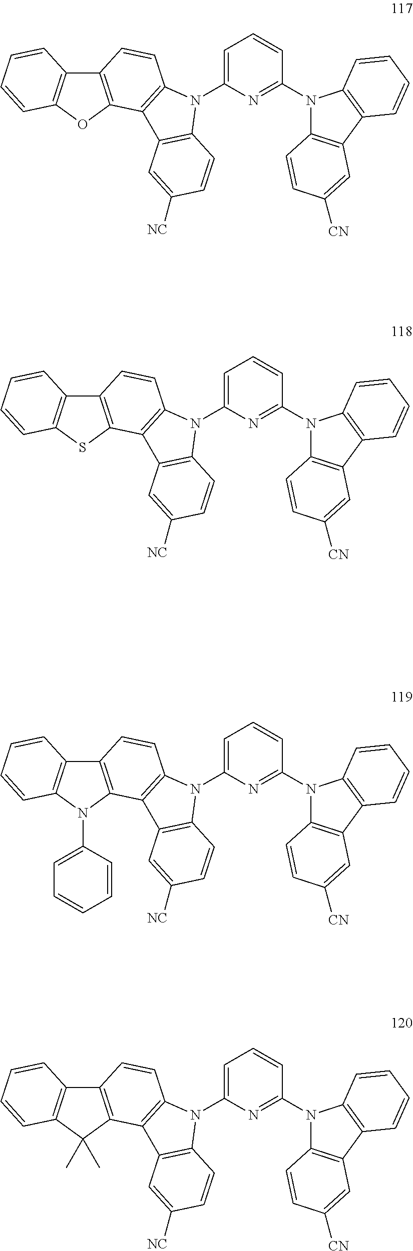 [SMILES: N#Cc1ccc2c(c1)c1ccccc1n2-c1cccc(-n2c3ccc(C#N)cc3c3c4oc5ccccc5c4ccc32)n1 ; N#Cc1ccc2c(c1)c1ccccc1n2-c1cccc(-n2c3ccc(C#N)cc3c3c4sc5ccccc5c4ccc32)n1 ; N#Cc1ccc2c(c1)c1ccccc1n2-c1cccc(-n2c3ccc(C#N)cc3c3c2ccc2c4ccccc4n(-c4ccccc4)c23)n1 ; CC1(C)c2ccccc2-c2ccc3c(c21)c1cc(C#N)ccc1n3-c1cccc(-n2c3ccccc3c3cc(C#N)ccc32)n1]

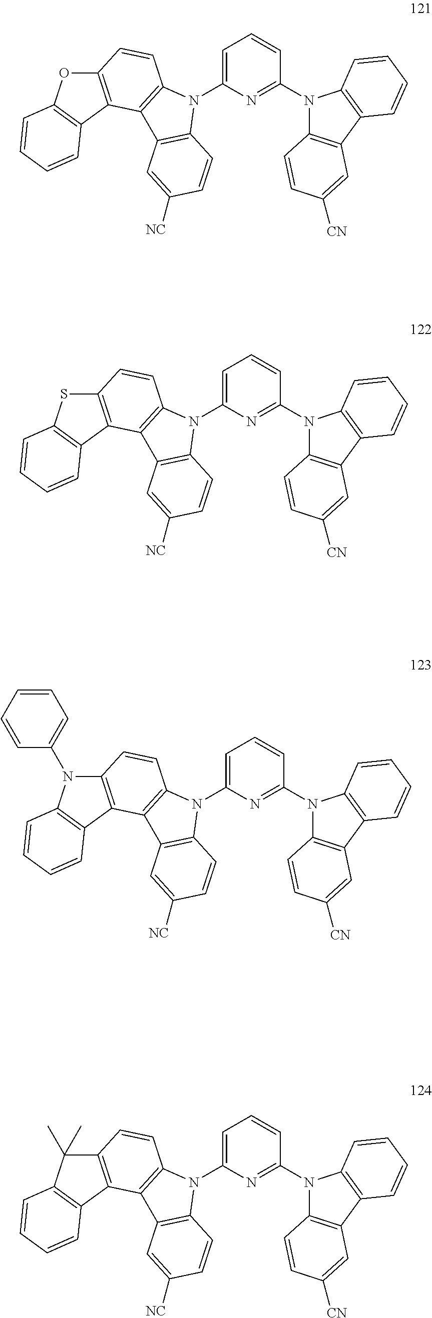 [SMILES: N#Cc1ccc2c(c1)c1ccccc1n2-c1cccc(-n2c3ccc(C#N)cc3c3c4c(ccc32)oc2ccccc24)n1 ; N#Cc1ccc2c(c1)c1ccccc1n2-c1cccc(-n2c3ccc(C#N)cc3c3c4c(ccc32)sc2ccccc24)n1 ; N#Cc1ccc2c(c1)c1ccccc1n2-c1cccc(-n2c3ccc(C#N)cc3c3c4c5ccccc5n(-c5ccccc5)c4ccc32)n1 ; CC1(C)c2ccccc2-c2c1ccc1c2c2cc(C#N)ccc2n1-c1cccc(-n2c3ccccc3c3cc(C#N)ccc32)n1]

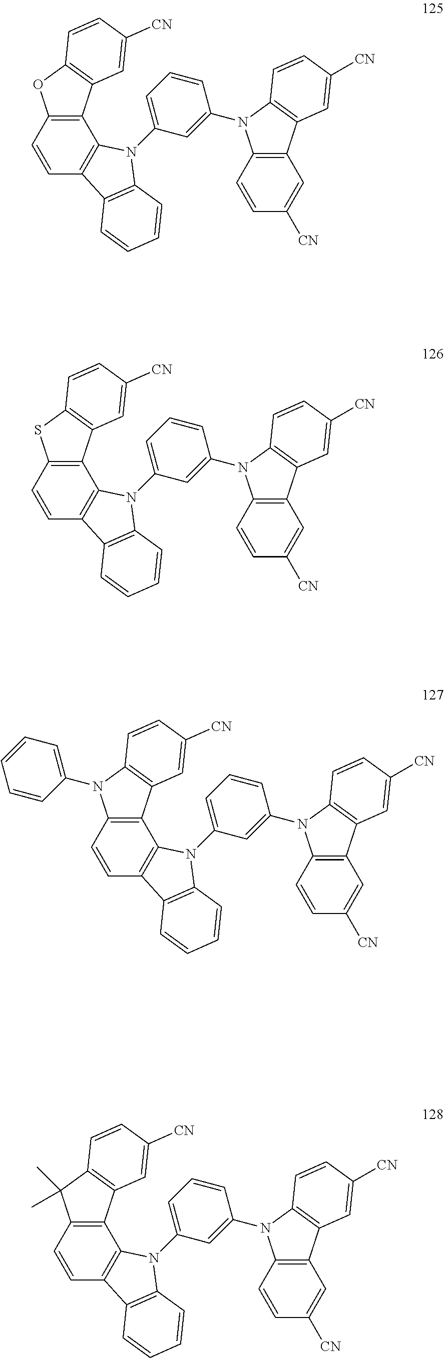 [SMILES: N#Cc1ccc2oc3ccc4c5ccccc5n(-c5cccc(-n6c7ccc(C#N)cc7c7cc(C#N)ccc76)c5)c4c3c2c1 ; N#Cc1ccc2sc3ccc4c5ccccc5n(-c5cccc(-n6c7ccc(C#N)cc7c7cc(C#N)ccc76)c5)c4c3c2c1 ; N#Cc1ccc2c(c1)c1cc(C#N)ccc1n2-c1cccc(-n2c3ccccc3c3ccc4c(c5cc(C#N)ccc5n4-c4ccccc4)c32)c1 ; CC1(C)c2ccc(C#N)cc2-c2c1ccc1c3ccccc3n(-c3cccc(-n4c5ccc(C#N)cc5c5cc(C#N)ccc54)c3)c21]

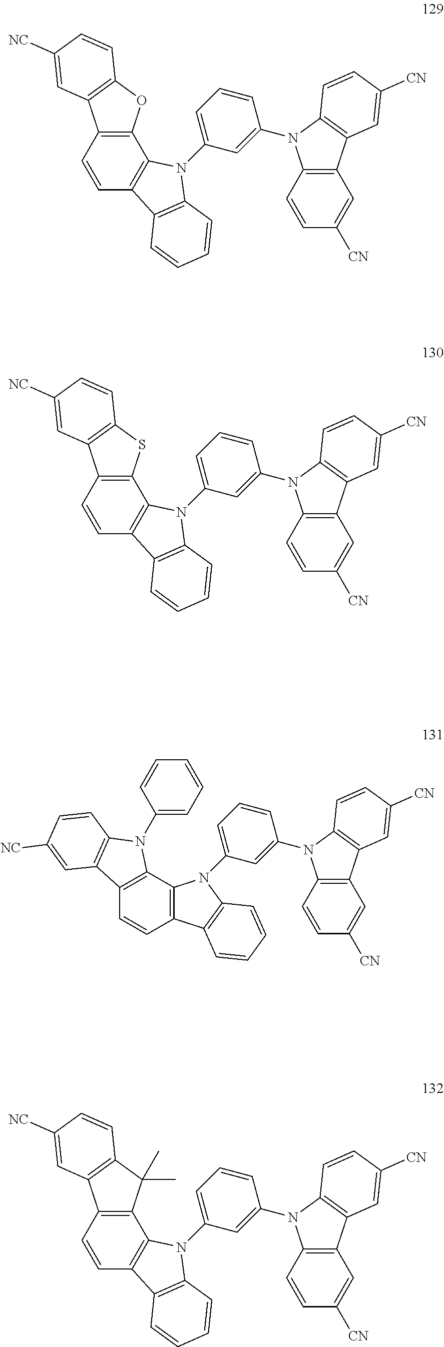 [SMILES: N#Cc1ccc2oc3c(ccc4c5ccccc5n(-c5cccc(-n6c7ccc(C#N)cc7c7cc(C#N)ccc76)c5)c43)c2c1 ; N#Cc1ccc2sc3c(ccc4c5ccccc5n(-c5cccc(-n6c7ccc(C#N)cc7c7cc(C#N)ccc76)c5)c43)c2c1 ; N#Cc1ccc2c(c1)c1cc(C#N)ccc1n2-c1cccc(-n2c3ccccc3c3ccc4c5cc(C#N)ccc5n(-c5ccccc5)c4c32)c1 ; CC1(C)c2ccc(C#N)cc2-c2ccc3c4ccccc4n(-c4cccc(-n5c6ccc(C#N)cc6c6cc(C#N)ccc65)c4)c3c21]

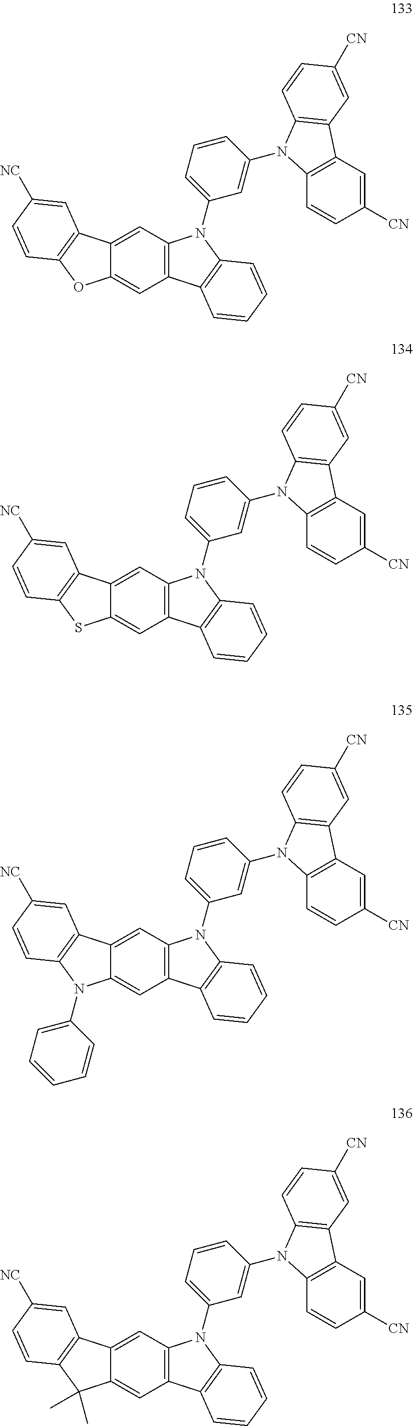 [SMILES: N#Cc1ccc2oc3cc4c5ccccc5n(-c5cccc(-n6c7ccc(C#N)cc7c7cc(C#N)ccc76)c5)c4cc3c2c1 ; N#Cc1ccc2sc3cc4c5ccccc5n(-c5cccc(-n6c7ccc(C#N)cc7c7cc(C#N)ccc76)c5)c4cc3c2c1 ; N#Cc1ccc2c(c1)c1cc(C#N)ccc1n2-c1cccc(-n2c3ccccc3c3cc4c(cc32)c2cc(C#N)ccc2n4-c2ccccc2)c1 ; CC1(C)c2ccc(C#N)cc2-c2cc3c(cc21)c1ccccc1n3-c1cccc(-n2c3ccc(C#N)cc3c3cc(C#N)ccc32)c1]

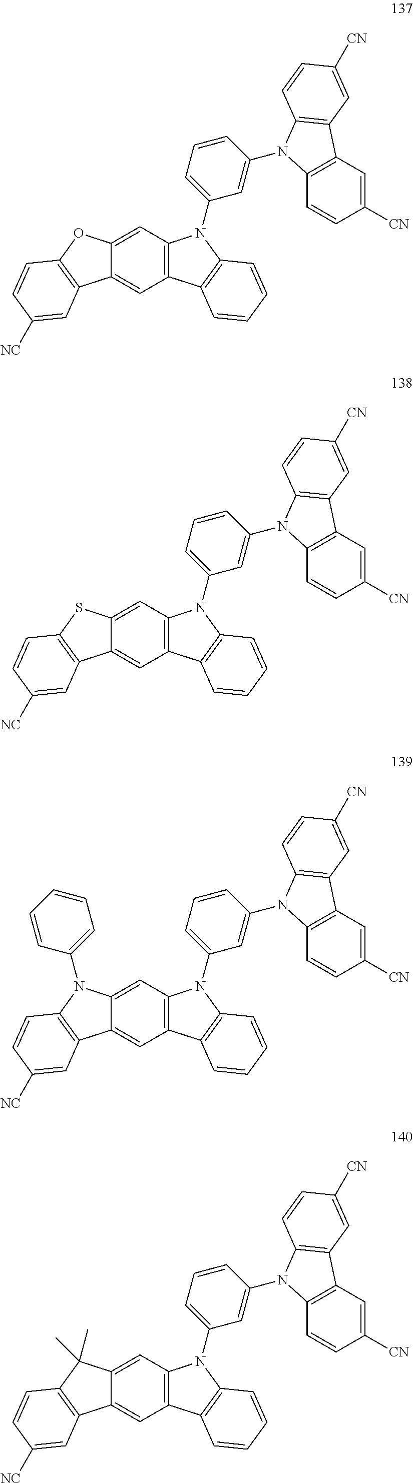 [SMILES: N#Cc1ccc2oc3cc4c(cc3c2c1)c1ccccc1n4-c1cccc(-n2c3ccc(C#N)cc3c3cc(C#N)ccc32)c1 ; N#Cc1ccc2sc3cc4c(cc3c2c1)c1ccccc1n4-c1cccc(-n2c3ccc(C#N)cc3c3cc(C#N)ccc32)c1 ; N#Cc1ccc2c(c1)c1cc(C#N)ccc1n2-c1cccc(-n2c3ccccc3c3cc4c5cc(C#N)ccc5n(-c5ccccc5)c4cc32)c1 ; CC1(C)c2ccc(C#N)cc2-c2cc3c4ccccc4n(-c4cccc(-n5c6ccc(C#N)cc6c6cc(C#N)ccc65)c4)c3cc21]

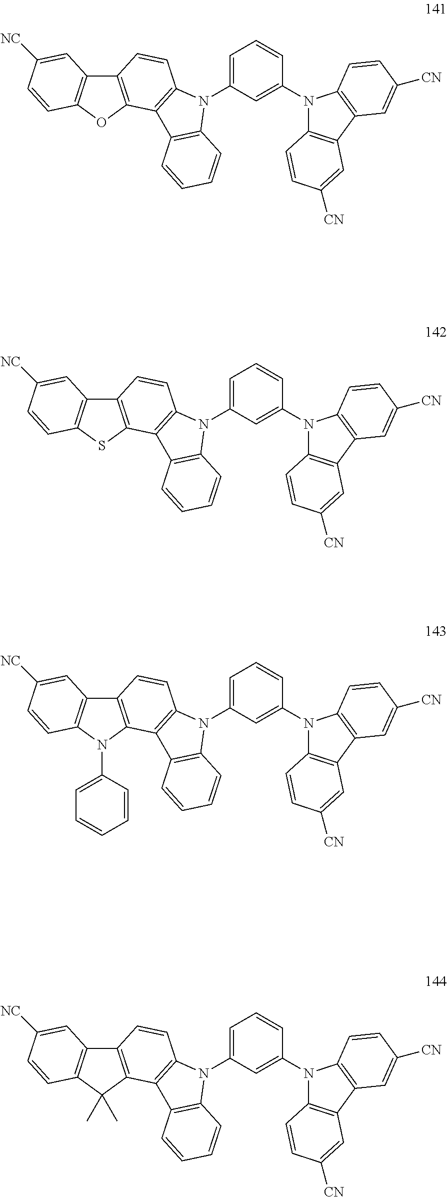 [SMILES: N#Cc1ccc2oc3c(ccc4c3c3ccccc3n4-c3cccc(-n4c5ccc(C#N)cc5c5cc(C#N)ccc54)c3)c2c1 ; N#Cc1ccc2sc3c(ccc4c3c3ccccc3n4-c3cccc(-n4c5ccc(C#N)cc5c5cc(C#N)ccc54)c3)c2c1 ; N#Cc1ccc2c(c1)c1cc(C#N)ccc1n2-c1cccc(-n2c3ccccc3c3c2ccc2c4cc(C#N)ccc4n(-c4ccccc4)c23)c1 ; CC1(C)c2ccc(C#N)cc2-c2ccc3c(c21)c1ccccc1n3-c1cccc(-n2c3ccc(C#N)cc3c3cc(C#N)ccc32)c1]

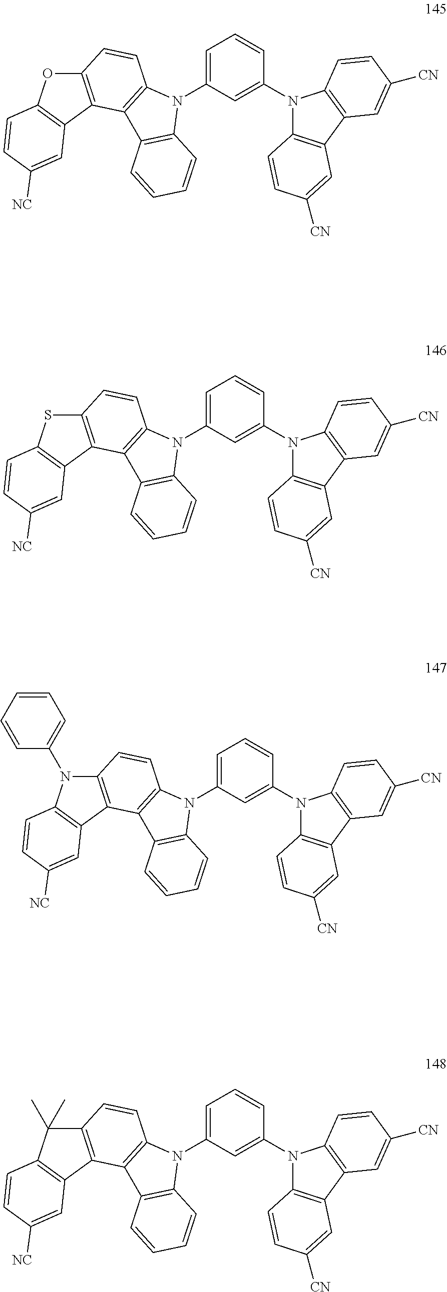 [SMILES: N#Cc1ccc2oc3ccc4c(c5ccccc5n4-c4cccc(-n5c6ccc(C#N)cc6c6cc(C#N)ccc65)c4)c3c2c1 ; N#Cc1ccc2sc3ccc4c(c5ccccc5n4-c4cccc(-n5c6ccc(C#N)cc6c6cc(C#N)ccc65)c4)c3c2c1 ; N#Cc1ccc2c(c1)c1cc(C#N)ccc1n2-c1cccc(-n2c3ccccc3c3c4c5cc(C#N)ccc5n(-c5ccccc5)c4ccc32)c1 ; CC1(C)c2ccc(C#N)cc2-c2c1ccc1c2c2ccccc2n1-c1cccc(-n2c3ccc(C#N)cc3c3cc(C#N)ccc32)c1]

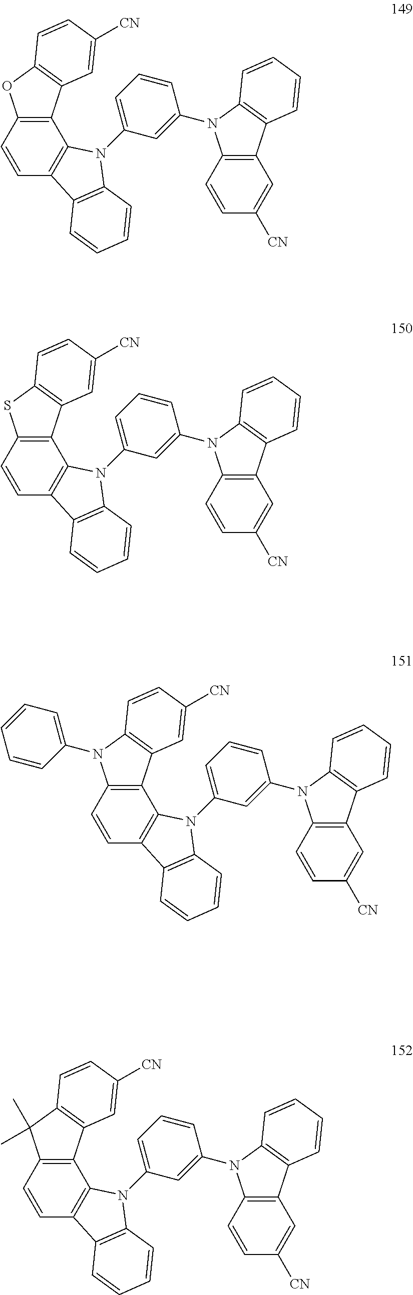 [SMILES: N#Cc1ccc2oc3ccc4c5ccccc5n(-c5cccc(-n6c7ccccc7c7cc(C#N)ccc76)c5)c4c3c2c1 ; N#Cc1ccc2sc3ccc4c5ccccc5n(-c5cccc(-n6c7ccccc7c7cc(C#N)ccc76)c5)c4c3c2c1 ; N#Cc1ccc2c(c1)c1ccccc1n2-c1cccc(-n2c3ccccc3c3ccc4c(c5cc(C#N)ccc5n4-c4ccccc4)c32)c1 ; CC1(C)c2ccc(C#N)cc2-c2c1ccc1c3ccccc3n(-c3cccc(-n4c5ccccc5c5cc(C#N)ccc54)c3)c21]

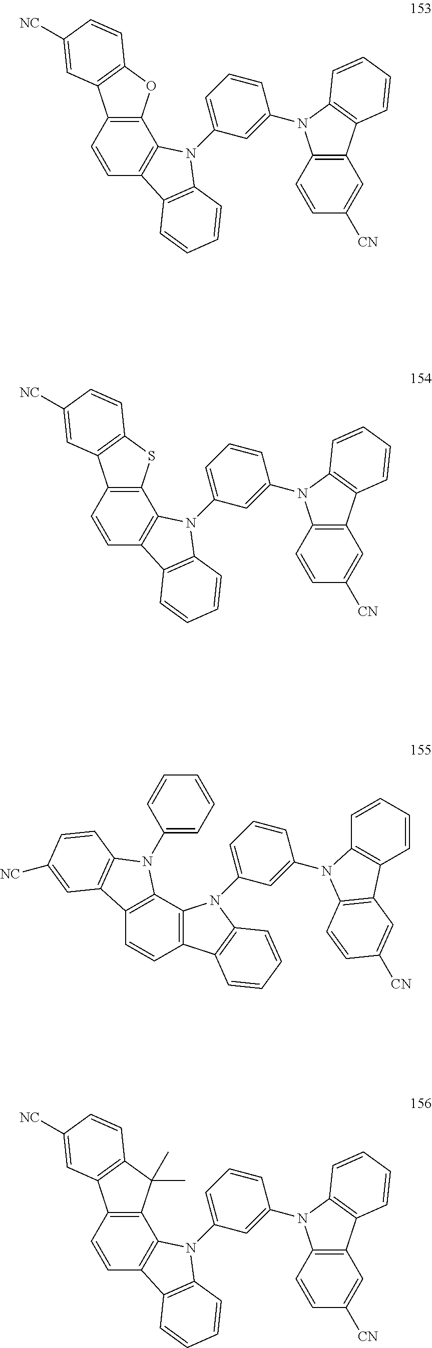 [SMILES: N#Cc1ccc2oc3c(ccc4c5ccccc5n(-c5cccc(-n6c7ccccc7c7cc(C#N)ccc76)c5)c43)c2c1 ; N#Cc1ccc2sc3c(ccc4c5ccccc5n(-c5cccc(-n6c7ccccc7c7cc(C#N)ccc76)c5)c43)c2c1 ; N#Cc1ccc2c(c1)c1ccccc1n2-c1cccc(-n2c3ccccc3c3ccc4c5cc(C#N)ccc5n(-c5ccccc5)c4c32)c1 ; CC1(C)c2ccc(C#N)cc2-c2ccc3c4ccccc4n(-c4cccc(-n5c6ccccc6c6cc(C#N)ccc65)c4)c3c21]

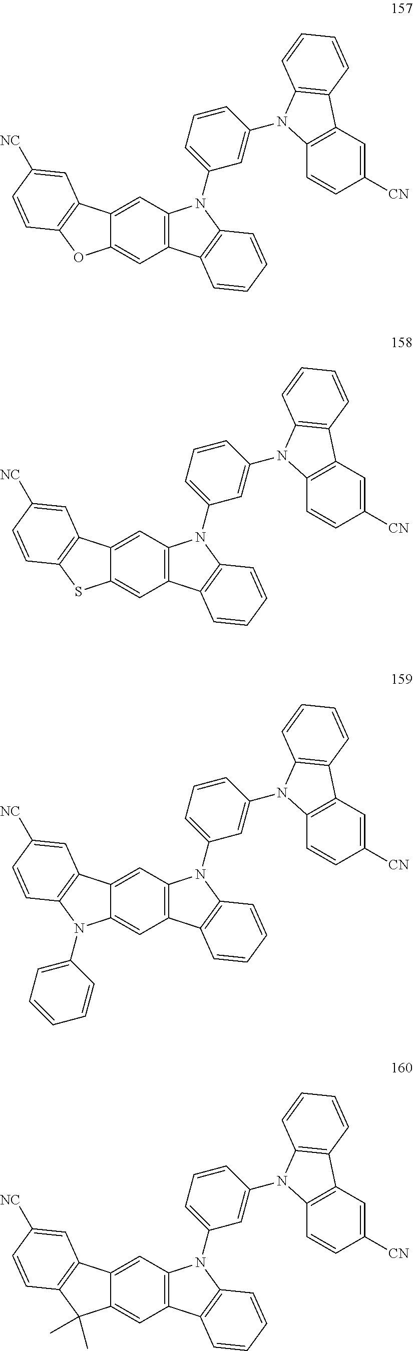 [SMILES: N#Cc1ccc2oc3cc4c5ccccc5n(-c5cccc(-n6c7ccccc7c7cc(C#N)ccc76)c5)c4cc3c2c1 ; N#Cc1ccc2sc3cc4c5ccccc5n(-c5cccc(-n6c7ccccc7c7cc(C#N)ccc76)c5)c4cc3c2c1 ; N#Cc1ccc2c(c1)c1ccccc1n2-c1cccc(-n2c3ccccc3c3cc4c(cc32)c2cc(C#N)ccc2n4-c2ccccc2)c1 ; CC1(C)c2ccc(C#N)cc2-c2cc3c(cc21)c1ccccc1n3-c1cccc(-n2c3ccccc3c3cc(C#N)ccc32)c1]

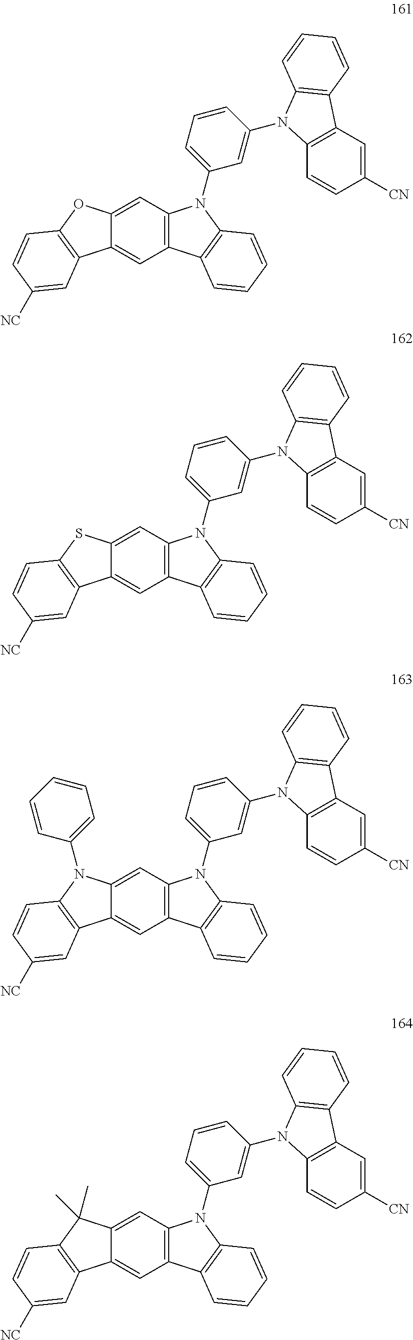 [SMILES: N#Cc1ccc2oc3cc4c(cc3c2c1)c1ccccc1n4-c1cccc(-n2c3ccccc3c3cc(C#N)ccc32)c1 ; N#Cc1ccc2sc3cc4c(cc3c2c1)c1ccccc1n4-c1cccc(-n2c3ccccc3c3cc(C#N)ccc32)c1 ; N#Cc1ccc2c(c1)c1ccccc1n2-c1cccc(-n2c3ccccc3c3cc4c5cc(C#N)ccc5n(-c5ccccc5)c4cc32)c1 ; CC1(C)c2ccc(C#N)cc2-c2cc3c4ccccc4n(-c4cccc(-n5c6ccccc6c6cc(C#N)ccc65)c4)c3cc21]

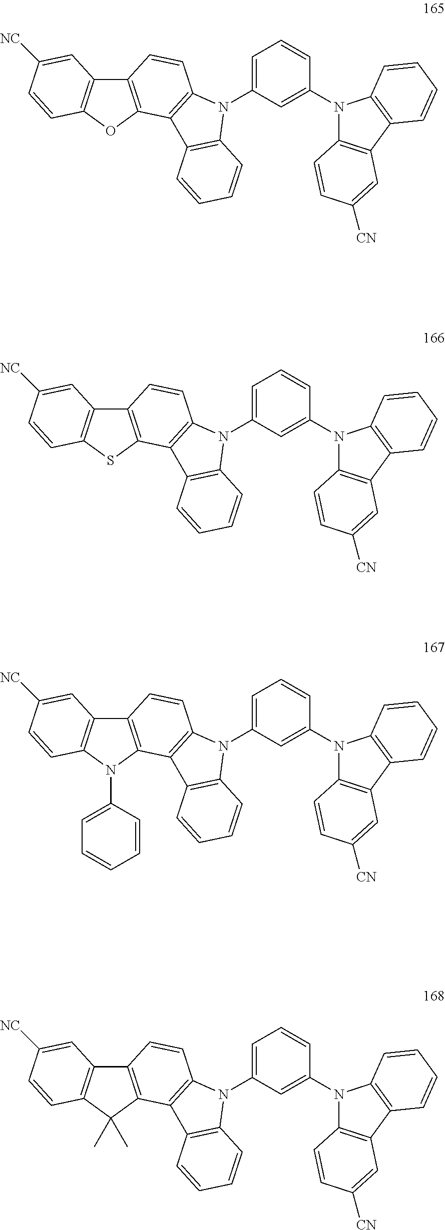 [SMILES: N#Cc1ccc2oc3c(ccc4c3c3ccccc3n4-c3cccc(-n4c5ccccc5c5cc(C#N)ccc54)c3)c2c1 ; N#Cc1ccc2sc3c(ccc4c3c3ccccc3n4-c3cccc(-n4c5ccccc5c5cc(C#N)ccc54)c3)c2c1 ; N#Cc1ccc2c(c1)c1ccccc1n2-c1cccc(-n2c3ccccc3c3c2ccc2c4cc(C#N)ccc4n(-c4ccccc4)c23)c1 ; CC1(C)c2ccc(C#N)cc2-c2ccc3c(c21)c1ccccc1n3-c1cccc(-n2c3ccccc3c3cc(C#N)ccc32)c1]

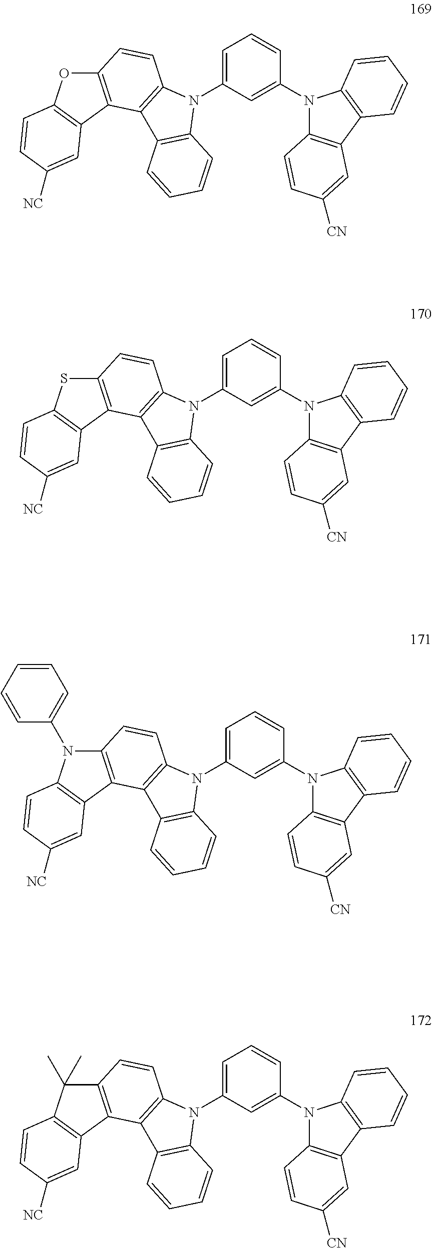 [SMILES: N#Cc1ccc2oc3ccc4c(c5ccccc5n4-c4cccc(-n5c6ccccc6c6cc(C#N)ccc65)c4)c3c2c1 ; N#Cc1ccc2sc3ccc4c(c5ccccc5n4-c4cccc(-n5c6ccccc6c6cc(C#N)ccc65)c4)c3c2c1 ; N#Cc1ccc2c(c1)c1ccccc1n2-c1cccc(-n2c3ccccc3c3c4c5cc(C#N)ccc5n(-c5ccccc5)c4ccc32)c1 ; CC1(C)c2ccc(C#N)cc2-c2c1ccc1c2c2ccccc2n1-c1cccc(-n2c3ccccc3c3cc(C#N)ccc32)c1]

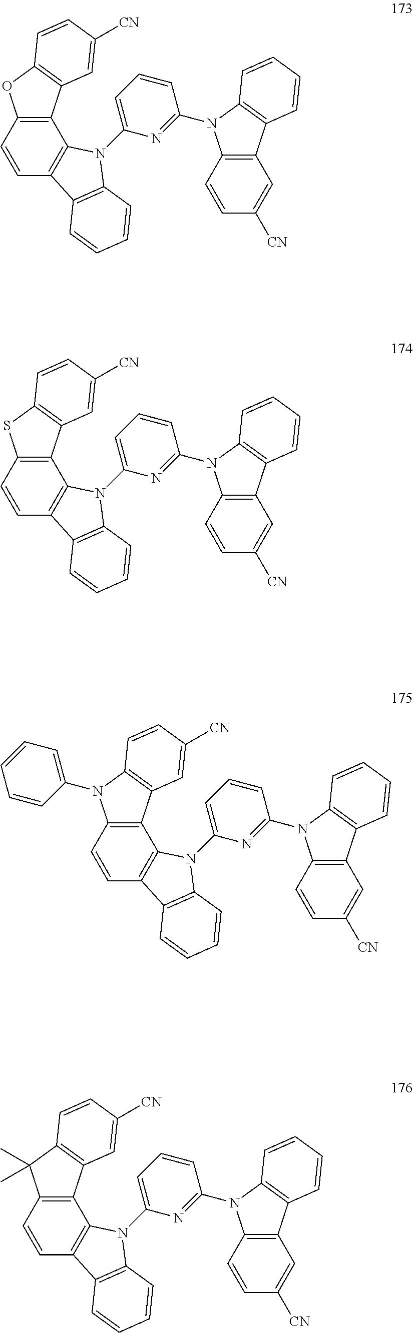 [SMILES: N#Cc1ccc2oc3ccc4c5ccccc5n(-c5cccc(-n6c7ccccc7c7cc(C#N)ccc76)n5)c4c3c2c1 ; N#Cc1ccc2sc3ccc4c5ccccc5n(-c5cccc(-n6c7ccccc7c7cc(C#N)ccc76)n5)c4c3c2c1 ; N#Cc1ccc2c(c1)c1ccccc1n2-c1cccc(-n2c3ccccc3c3ccc4c(c5cc(C#N)ccc5n4-c4ccccc4)c32)n1 ; CC1(C)c2ccc(C#N)cc2-c2c1ccc1c3ccccc3n(-c3cccc(-n4c5ccccc5c5cc(C#N)ccc54)n3)c21]

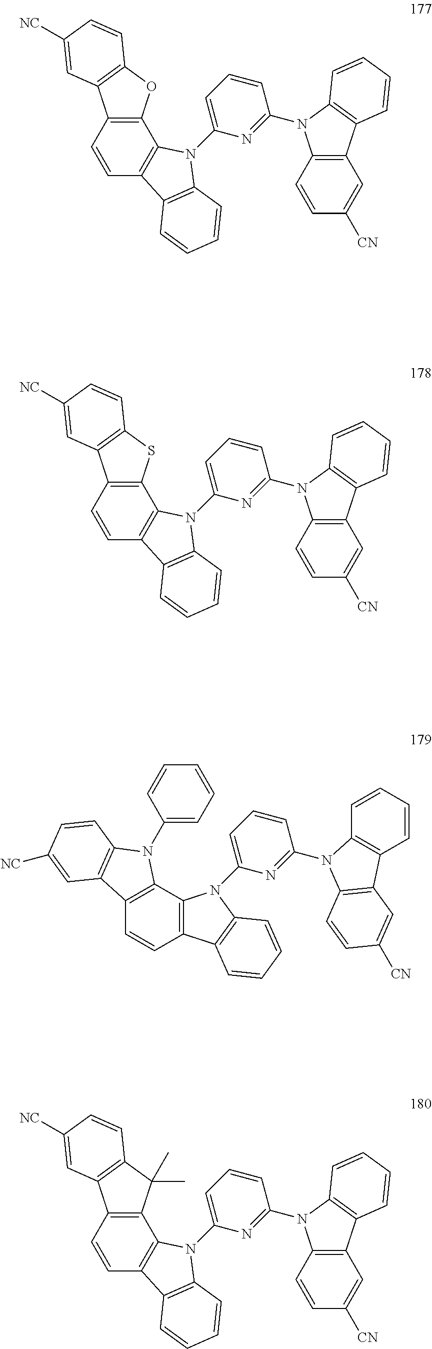 [SMILES: N#Cc1ccc2oc3c(ccc4c5ccccc5n(-c5cccc(-n6c7ccccc7c7cc(C#N)ccc76)n5)c43)c2c1 ; N#Cc1ccc2sc3c(ccc4c5ccccc5n(-c5cccc(-n6c7ccccc7c7cc(C#N)ccc76)n5)c43)c2c1 ; N#Cc1ccc2c(c1)c1ccccc1n2-c1cccc(-n2c3ccccc3c3ccc4c5cc(C#N)ccc5n(-c5ccccc5)c4c32)n1 ; CC1(C)c2ccc(C#N)cc2-c2ccc3c4ccccc4n(-c4cccc(-n5c6ccccc6c6cc(C#N)ccc65)n4)c3c21]

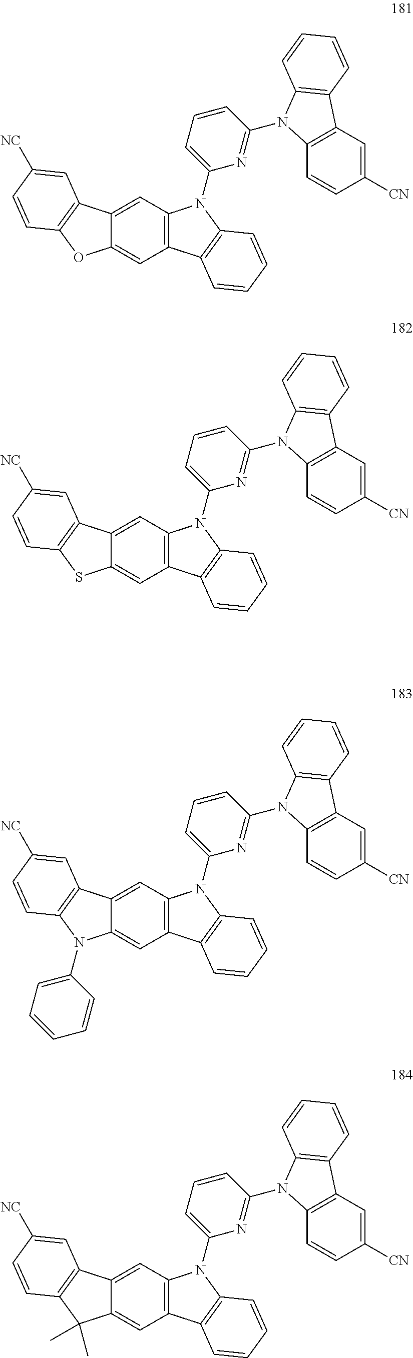 [SMILES: N#Cc1ccc2oc3cc4c5ccccc5n(-c5cccc(-n6c7ccccc7c7cc(C#N)ccc76)n5)c4cc3c2c1 ; N#Cc1ccc2sc3cc4c5ccccc5n(-c5cccc(-n6c7ccccc7c7cc(C#N)ccc76)n5)c4cc3c2c1 ; N#Cc1ccc2c(c1)c1cc3c(cc1n2-c1ccccc1)c1ccccc1n3-c1cccc(-n2c3ccccc3c3cc(C#N)ccc32)n1 ; CC1(C)c2ccc(C#N)cc2-c2cc3c(cc21)c1ccccc1n3-c1cccc(-n2c3ccccc3c3cc(C#N)ccc32)n1]

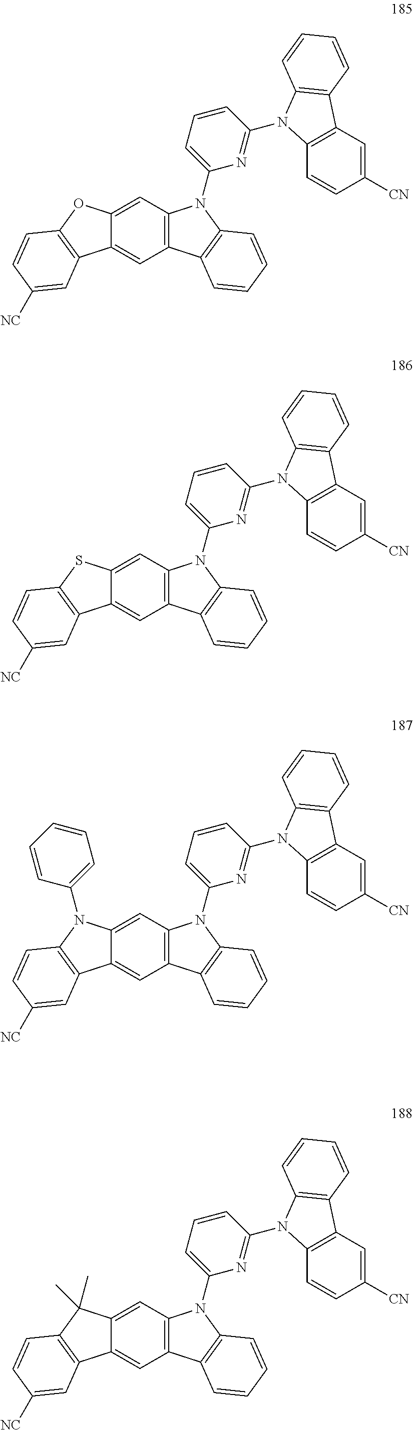 [SMILES: N#Cc1ccc2oc3cc4c(cc3c2c1)c1ccccc1n4-c1cccc(-n2c3ccccc3c3cc(C#N)ccc32)n1 ; N#Cc1ccc2sc3cc4c(cc3c2c1)c1ccccc1n4-c1cccc(-n2c3ccccc3c3cc(C#N)ccc32)n1 ; N#Cc1ccc2c(c1)c1cc3c4ccccc4n(-c4cccc(-n5c6ccccc6c6cc(C#N)ccc65)n4)c3cc1n2-c1ccccc1 ; CC1(C)c2ccc(C#N)cc2-c2cc3c4ccccc4n(-c4cccc(-n5c6ccccc6c6cc(C#N)ccc65)n4)c3cc21]

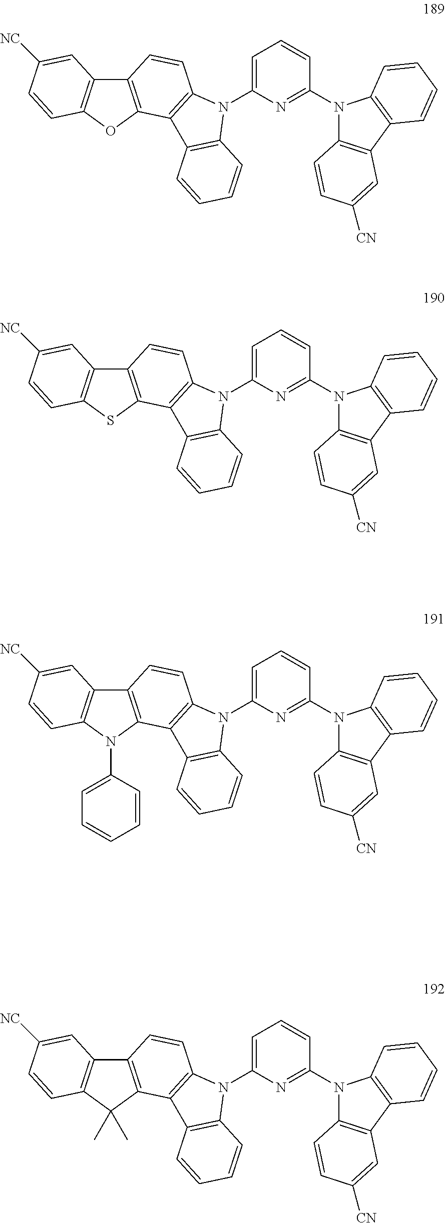 [SMILES: N#Cc1ccc2oc3c(ccc4c3c3ccccc3n4-c3cccc(-n4c5ccccc5c5cc(C#N)ccc54)n3)c2c1 ; N#Cc1ccc2sc3c(ccc4c3c3ccccc3n4-c3cccc(-n4c5ccccc5c5cc(C#N)ccc54)n3)c2c1 ; N#Cc1ccc2c(c1)c1ccccc1n2-c1cccc(-n2c3ccccc3c3c2ccc2c4cc(C#N)ccc4n(-c4ccccc4)c23)n1 ; CC1(C)c2ccc(C#N)cc2-c2ccc3c(c21)c1ccccc1n3-c1cccc(-n2c3ccccc3c3cc(C#N)ccc32)n1]

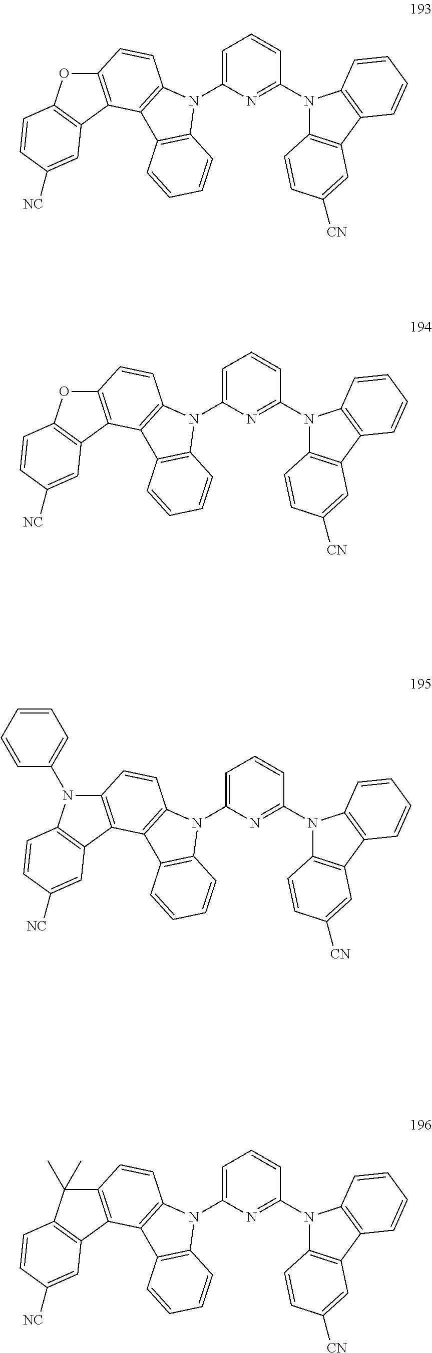 [SMILES: N#Cc1ccc2oc3ccc4c(c5ccccc5n4-c4cccc(-n5c6ccccc6c6cc(C#N)ccc65)n4)c3c2c1 ; N#Cc1ccc2oc3ccc4c(c5ccccc5n4-c4cccc(-n5c6ccccc6c6cc(C#N)ccc65)n4)c3c2c1 ; N#Cc1ccc2c(c1)c1ccccc1n2-c1cccc(-n2c3ccccc3c3c4c5cc(C#N)ccc5n(-c5ccccc5)c4ccc32)n1 ; CC1(C)c2ccc(C#N)cc2-c2c1ccc1c2c2ccccc2n1-c1cccc(-n2c3ccccc3c3cc(C#N)ccc32)n1]

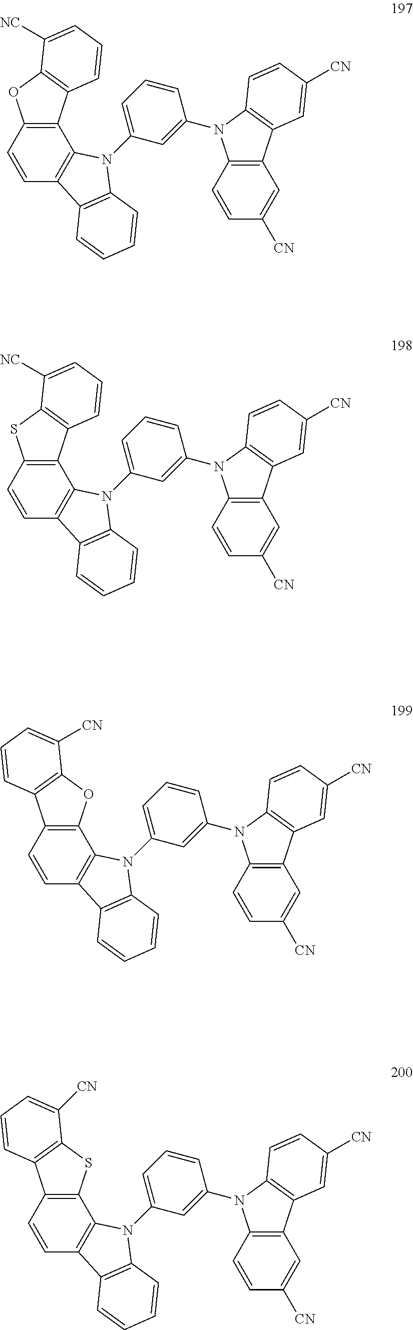 [SMILES: N#Cc1ccc2c(c1)c1cc(C#N)ccc1n2-c1cccc(-n2c3ccccc3c3ccc4oc5c(C#N)cccc5c4c32)c1 ; N#Cc1ccc2c(c1)c1cc(C#N)ccc1n2-c1cccc(-n2c3ccccc3c3ccc4sc5c(C#N)cccc5c4c32)c1 ; N#Cc1ccc2c(c1)c1cc(C#N)ccc1n2-c1cccc(-n2c3ccccc3c3ccc4c5cccc(C#N)c5oc4c32)c1 ; N#Cc1ccc2c(c1)c1cc(C#N)ccc1n2-c1cccc(-n2c3ccccc3c3ccc4c5cccc(C#N)c5sc4c32)c1]

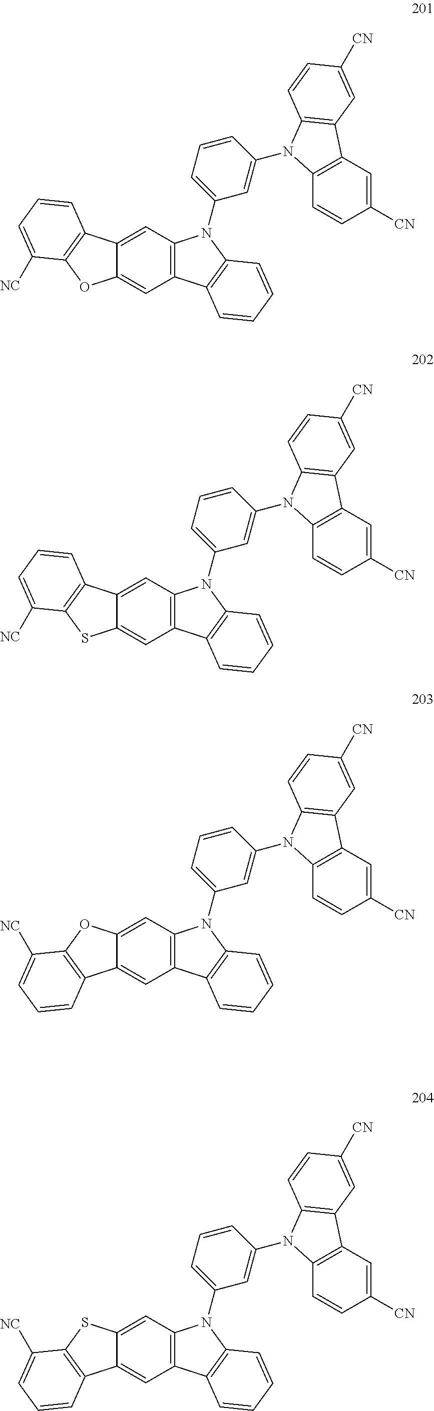 [SMILES: N#Cc1ccc2c(c1)c1cc(C#N)ccc1n2-c1cccc(-n2c3ccccc3c3cc4oc5c(C#N)cccc5c4cc32)c1 ; N#Cc1ccc2c(c1)c1cc(C#N)ccc1n2-c1cccc(-n2c3ccccc3c3cc4sc5c(C#N)cccc5c4cc32)c1 ; N#Cc1ccc2c(c1)c1cc(C#N)ccc1n2-c1cccc(-n2c3ccccc3c3cc4c(cc32)oc2c(C#N)cccc24)c1 ; N#Cc1ccc2c(c1)c1cc(C#N)ccc1n2-c1cccc(-n2c3ccccc3c3cc4c(cc32)sc2c(C#N)cccc24)c1]

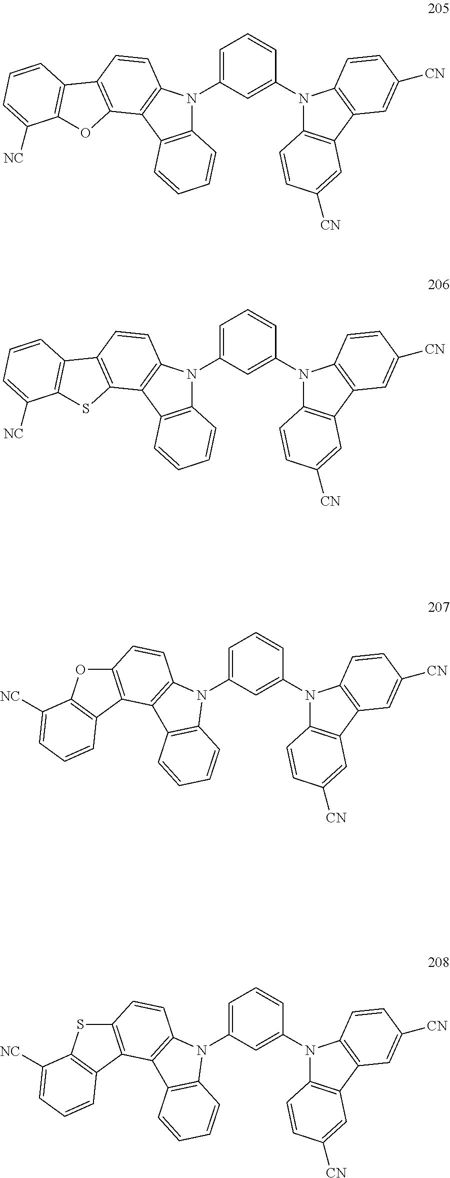 [SMILES: N#Cc1ccc2c(c1)c1cc(C#N)ccc1n2-c1cccc(-n2c3ccccc3c3c4oc5c(C#N)cccc5c4ccc32)c1 ; N#Cc1ccc2c(c1)c1cc(C#N)ccc1n2-c1cccc(-n2c3ccccc3c3c4sc5c(C#N)cccc5c4ccc32)c1 ; N#Cc1ccc2c(c1)c1cc(C#N)ccc1n2-c1cccc(-n2c3ccccc3c3c4c(ccc32)oc2c(C#N)cccc24)c1 ; N#Cc1ccc2c(c1)c1cc(C#N)ccc1n2-c1cccc(-n2c3ccccc3c3c4c(ccc32)sc2c(C#N)cccc24)c1]

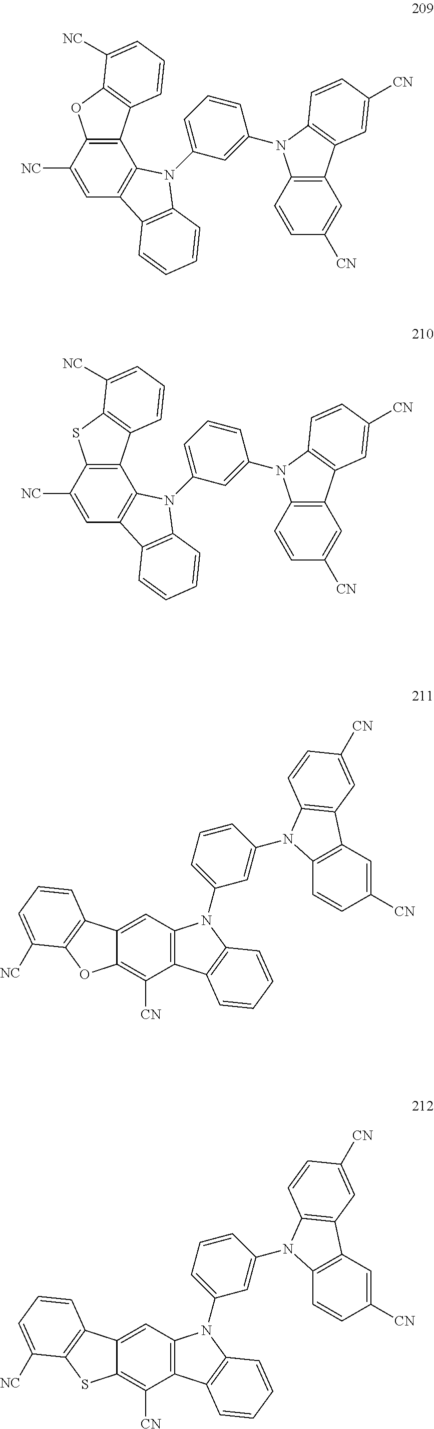 [SMILES: N#Cc1ccc2c(c1)c1cc(C#N)ccc1n2-c1cccc(-n2c3ccccc3c3cc(C#N)c4oc5c(C#N)cccc5c4c32)c1 ; N#Cc1ccc2c(c1)c1cc(C#N)ccc1n2-c1cccc(-n2c3ccccc3c3cc(C#N)c4sc5c(C#N)cccc5c4c32)c1 ; N#Cc1ccc2c(c1)c1cc(C#N)ccc1n2-c1cccc(-n2c3ccccc3c3c(C#N)c4oc5c(C#N)cccc5c4cc32)c1 ; N#Cc1ccc2c(c1)c1cc(C#N)ccc1n2-c1cccc(-n2c3ccccc3c3c(C#N)c4sc5c(C#N)cccc5c4cc32)c1]

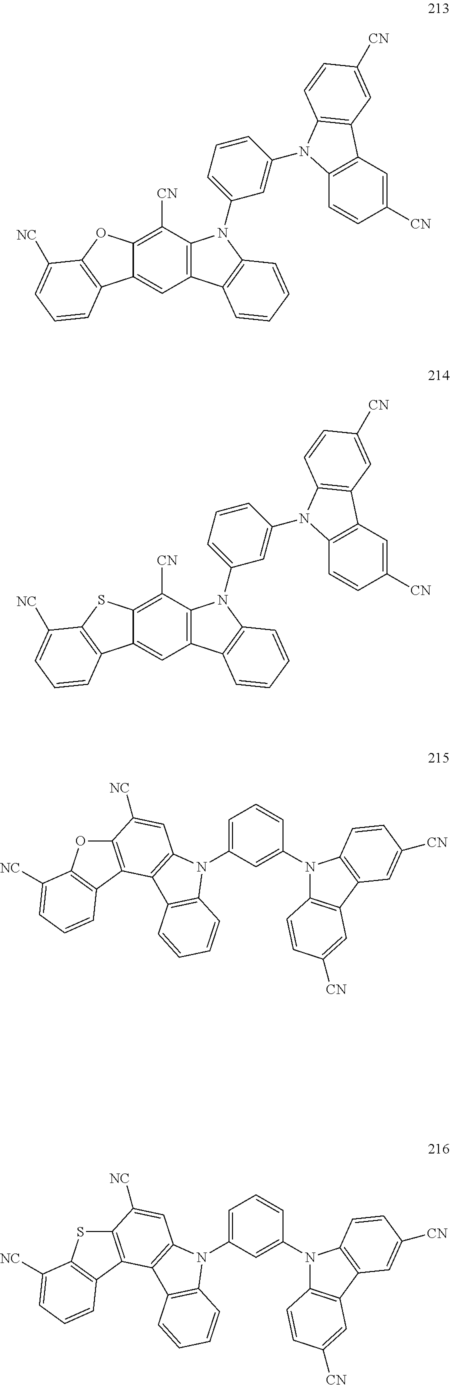 [SMILES: N#Cc1ccc2c(c1)c1cc(C#N)ccc1n2-c1cccc(-n2c3ccccc3c3cc4c(oc5c(C#N)cccc54)c(C#N)c32)c1 ; N#Cc1ccc2c(c1)c1cc(C#N)ccc1n2-c1cccc(-n2c3ccccc3c3cc4c(sc5c(C#N)cccc54)c(C#N)c32)c1 ; N#Cc1ccc2c(c1)c1cc(C#N)ccc1n2-c1cccc(-n2c3ccccc3c3c4c(oc5c(C#N)cccc54)c(C#N)cc32)c1 ; N#Cc1ccc2c(c1)c1cc(C#N)ccc1n2-c1cccc(-n2c3ccccc3c3c4c(sc5c(C#N)cccc54)c(C#N)cc32)c1]

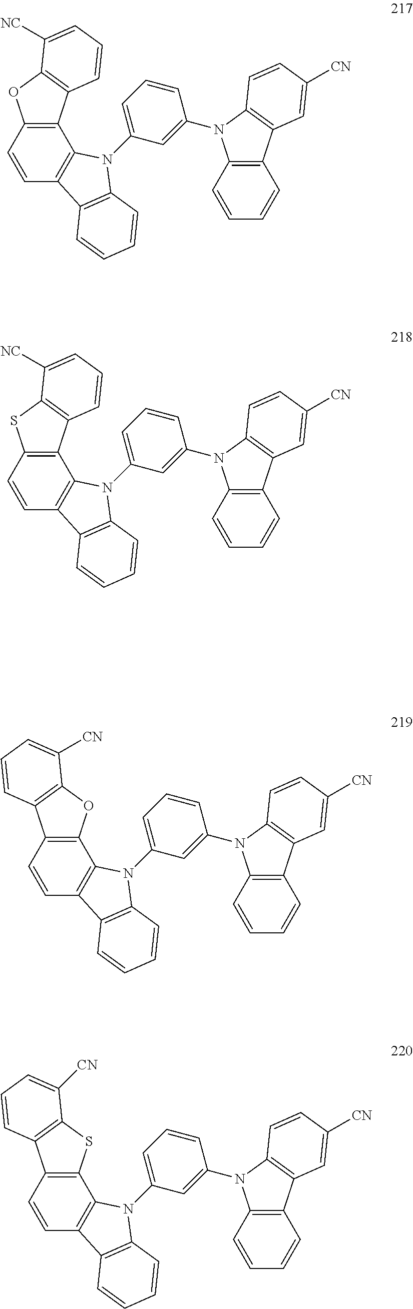 [SMILES: N#Cc1ccc2c(c1)c1ccccc1n2-c1cccc(-n2c3ccccc3c3ccc4oc5c(C#N)cccc5c4c32)c1 ; N#Cc1ccc2c(c1)c1ccccc1n2-c1cccc(-n2c3ccccc3c3ccc4sc5c(C#N)cccc5c4c32)c1 ; N#Cc1ccc2c(c1)c1ccccc1n2-c1cccc(-n2c3ccccc3c3ccc4c5cccc(C#N)c5oc4c32)c1 ; N#Cc1ccc2c(c1)c1ccccc1n2-c1cccc(-n2c3ccccc3c3ccc4c5cccc(C#N)c5sc4c32)c1]

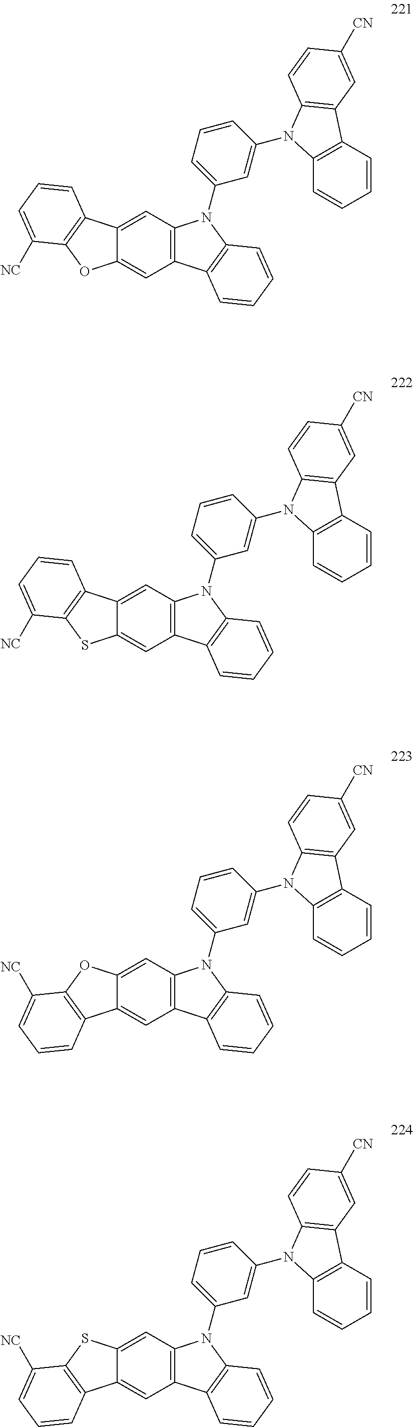 [SMILES: N#Cc1ccc2c(c1)c1ccccc1n2-c1cccc(-n2c3ccccc3c3cc4oc5c(C#N)cccc5c4cc32)c1 ; N#Cc1ccc2c(c1)c1ccccc1n2-c1cccc(-n2c3ccccc3c3cc4sc5c(C#N)cccc5c4cc32)c1 ; N#Cc1ccc2c(c1)c1ccccc1n2-c1cccc(-n2c3ccccc3c3cc4c(cc32)oc2c(C#N)cccc24)c1 ; N#Cc1ccc2c(c1)c1ccccc1n2-c1cccc(-n2c3ccccc3c3cc4c(cc32)sc2c(C#N)cccc24)c1]

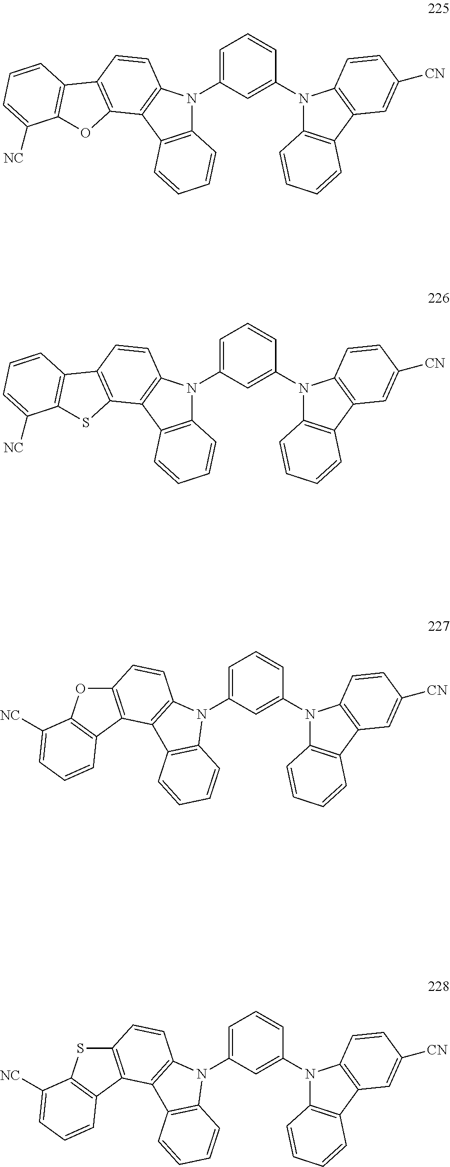 [SMILES: N#Cc1ccc2c(c1)c1ccccc1n2-c1cccc(-n2c3ccccc3c3c4oc5c(C#N)cccc5c4ccc32)c1 ; N#Cc1ccc2c(c1)c1ccccc1n2-c1cccc(-n2c3ccccc3c3c4sc5c(C#N)cccc5c4ccc32)c1 ; N#Cc1ccc2c(c1)c1ccccc1n2-c1cccc(-n2c3ccccc3c3c4c(ccc32)oc2c(C#N)cccc24)c1 ; N#Cc1ccc2c(c1)c1ccccc1n2-c1cccc(-n2c3ccccc3c3c4c(ccc32)sc2c(C#N)cccc24)c1]

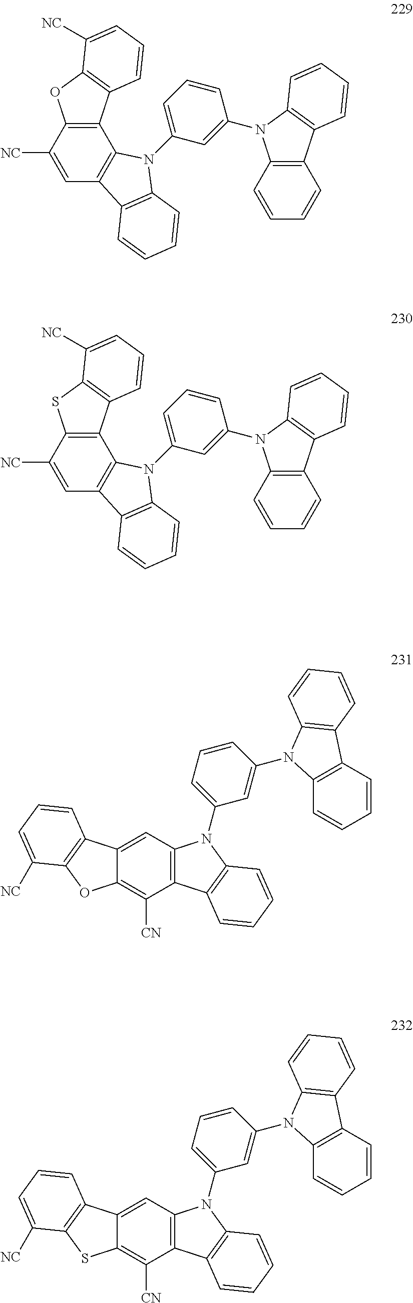 [SMILES: N#Cc1cccc2c1oc1c(C#N)cc3c4ccccc4n(-c4cccc(-n5c6ccccc6c6ccccc65)c4)c3c12 ; N#Cc1cccc2c1sc1c(C#N)cc3c4ccccc4n(-c4cccc(-n5c6ccccc6c6ccccc65)c4)c3c12 ; N#Cc1cccc2c1oc1c(C#N)c3c4ccccc4n(-c4cccc(-n5c6ccccc6c6ccccc65)c4)c3cc12 ; N#Cc1cccc2c1sc1c(C#N)c3c4ccccc4n(-c4cccc(-n5c6ccccc6c6ccccc65)c4)c3cc12]

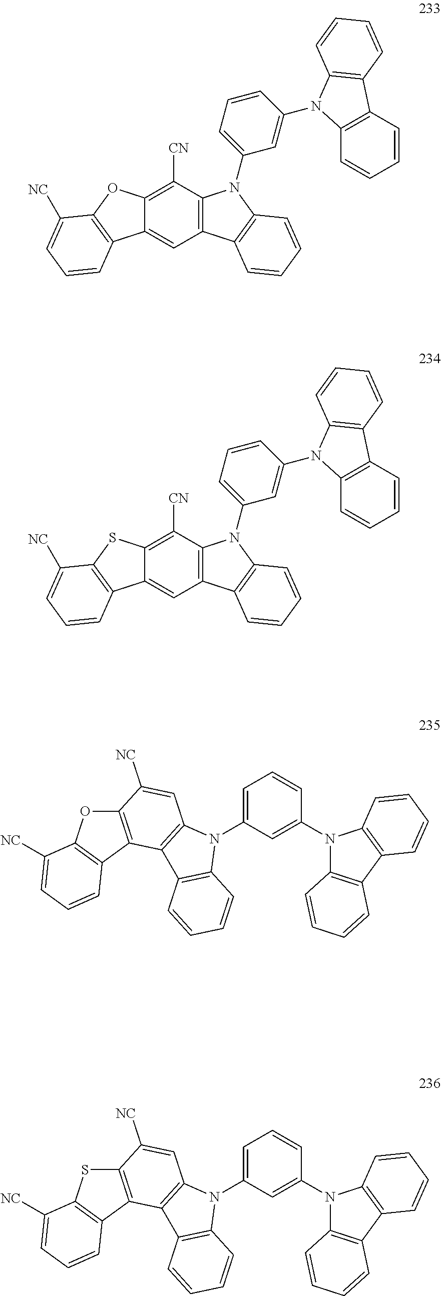 [SMILES: N#Cc1cccc2c1oc1c(C#N)c3c(cc12)c1ccccc1n3-c1cccc(-n2c3ccccc3c3ccccc32)c1 ; N#Cc1cccc2c1sc1c(C#N)c3c(cc12)c1ccccc1n3-c1cccc(-n2c3ccccc3c3ccccc32)c1 ; N#Cc1cccc2c1oc1c(C#N)cc3c(c4ccccc4n3-c3cccc(-n4c5ccccc5c5ccccc54)c3)c12 ; N#Cc1cccc2c1sc1c(C#N)cc3c(c4ccccc4n3-c3cccc(-n4c5ccccc5c5ccccc54)c3)c12]

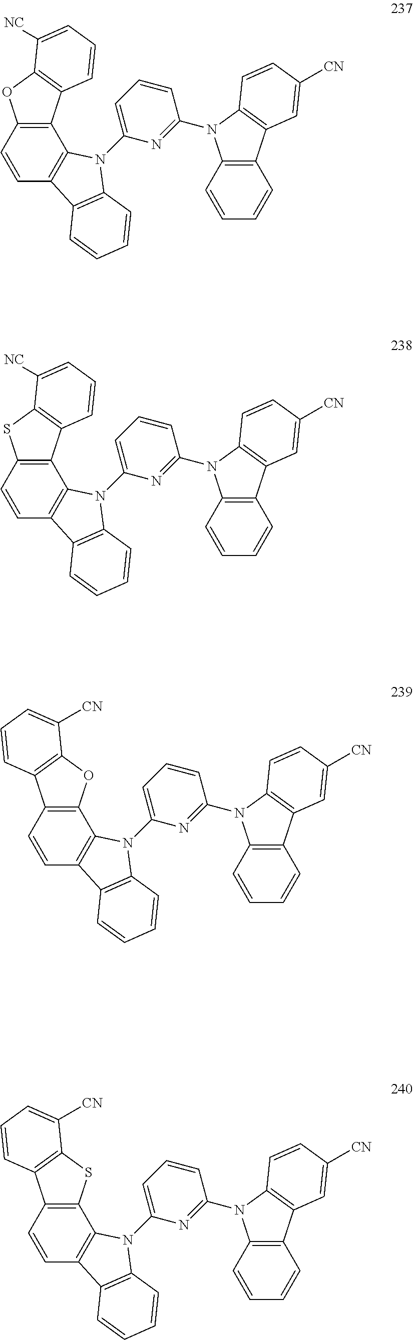

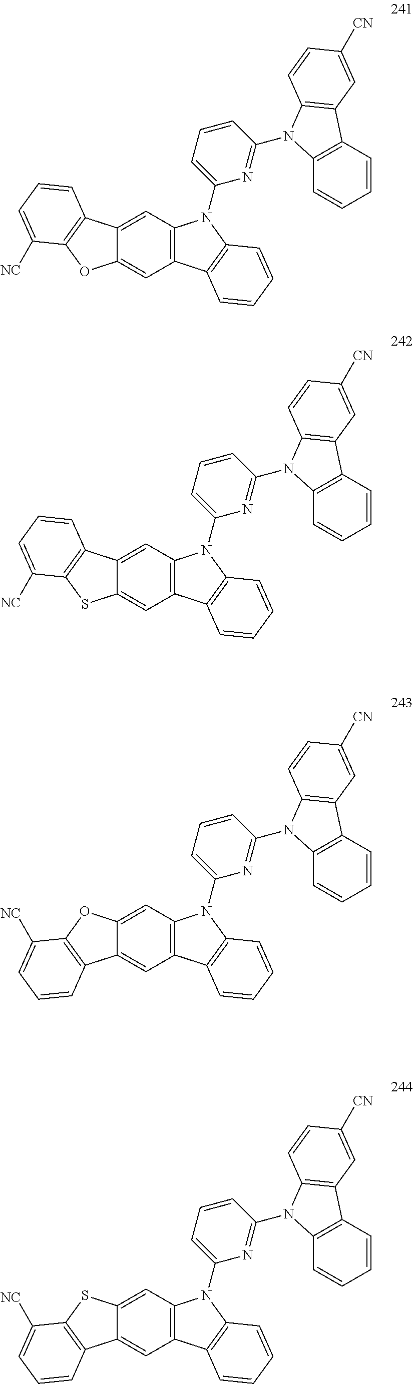 [SMILES: N#Cc1ccc2c(c1)c1ccccc1n2-c1cccc(-n2c3ccccc3c3cc4oc5c(C#N)cccc5c4cc32)n1 ; N#Cc1ccc2c(c1)c1ccccc1n2-c1cccc(-n2c3ccccc3c3cc4sc5c(C#N)cccc5c4cc32)n1 ; N#Cc1ccc2c(c1)c1ccccc1n2-c1cccc(-n2c3ccccc3c3cc4c(cc32)oc2c(C#N)cccc24)n1 ; N#Cc1ccc2c(c1)c1ccccc1n2-c1cccc(-n2c3ccccc3c3cc4c(cc32)sc2c(C#N)cccc24)n1]

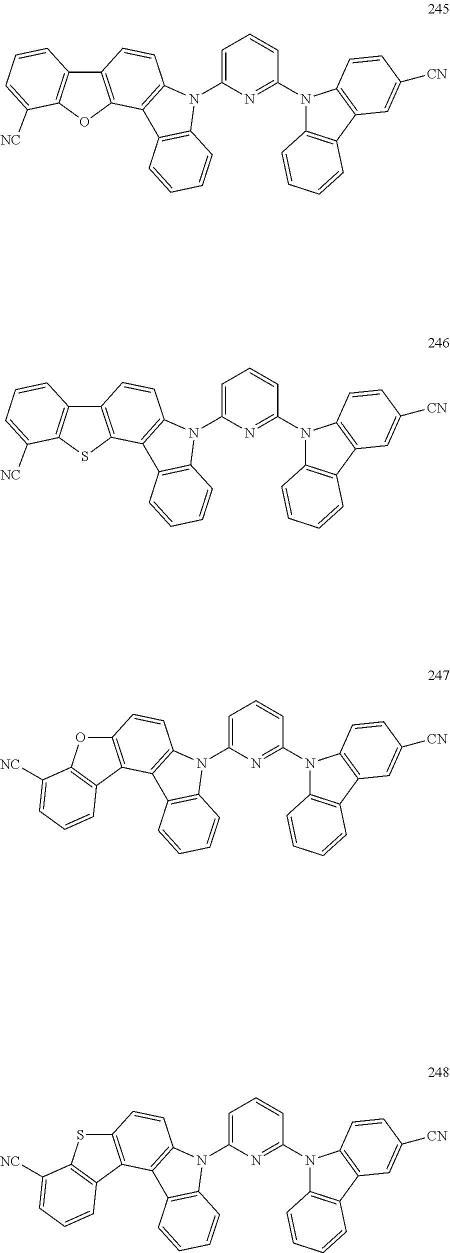 [SMILES: N#Cc1ccc2c(c1)c1ccccc1n2-c1cccc(-n2c3ccccc3c3c4oc5c(C#N)cccc5c4ccc32)n1 ; N#Cc1ccc2c(c1)c1ccccc1n2-c1cccc(-n2c3ccccc3c3c4sc5c(C#N)cccc5c4ccc32)n1 ; N#Cc1ccc2c(c1)c1ccccc1n2-c1cccc(-n2c3ccccc3c3c4c(ccc32)oc2c(C#N)cccc24)n1 ; N#Cc1ccc2c(c1)c1ccccc1n2-c1cccc(-n2c3ccccc3c3c4c(ccc32)sc2c(C#N)cccc24)n1]

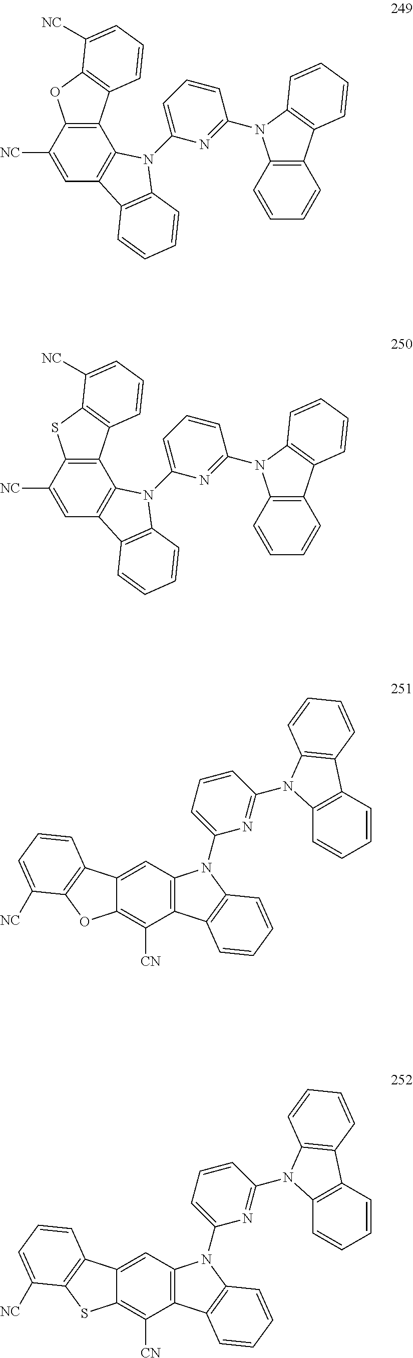 [SMILES: N#Cc1cccc2c1oc1c(C#N)cc3c4ccccc4n(-c4cccc(-n5c6ccccc6c6ccccc65)n4)c3c12 ; N#Cc1cccc2c1sc1c(C#N)cc3c4ccccc4n(-c4cccc(-n5c6ccccc6c6ccccc65)n4)c3c12 ; N#Cc1cccc2c1oc1c(C#N)c3c4ccccc4n(-c4cccc(-n5c6ccccc6c6ccccc65)n4)c3cc12 ; N#Cc1cccc2c1sc1c(C#N)c3c4ccccc4n(-c4cccc(-n5c6ccccc6c6ccccc65)n4)c3cc12]

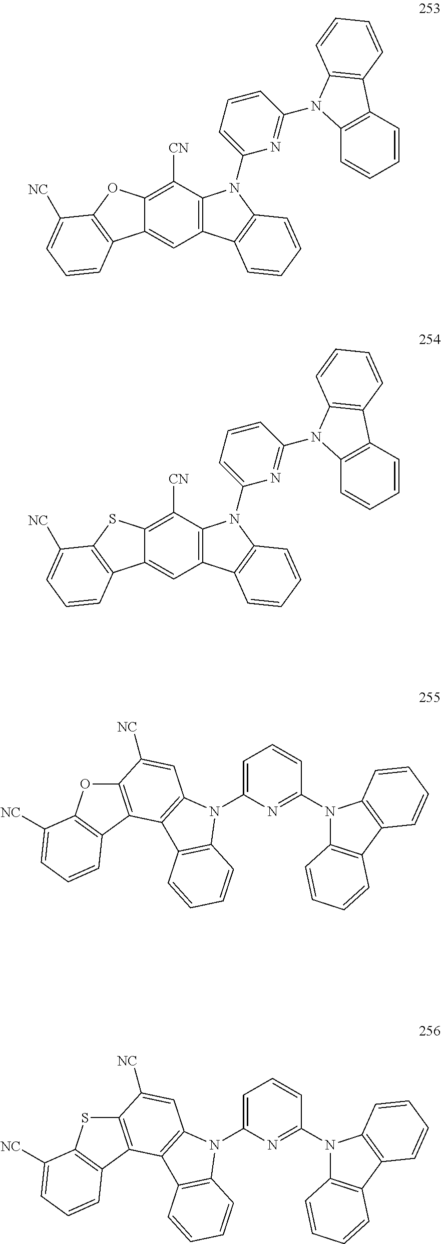 [SMILES: N#Cc1cccc2c1oc1c(C#N)c3c(cc12)c1ccccc1n3-c1cccc(-n2c3ccccc3c3ccccc32)n1 ; N#Cc1cccc2c1sc1c(C#N)c3c(cc12)c1ccccc1n3-c1cccc(-n2c3ccccc3c3ccccc32)n1 ; N#Cc1cccc2c1oc1c(C#N)cc3c(c4ccccc4n3-c3cccc(-n4c5ccccc5c5ccccc54)n3)c12 ; N#Cc1cccc2c1sc1c(C#N)cc3c(c4ccccc4n3-c3cccc(-n4c5ccccc5c5ccccc54)n3)c12]

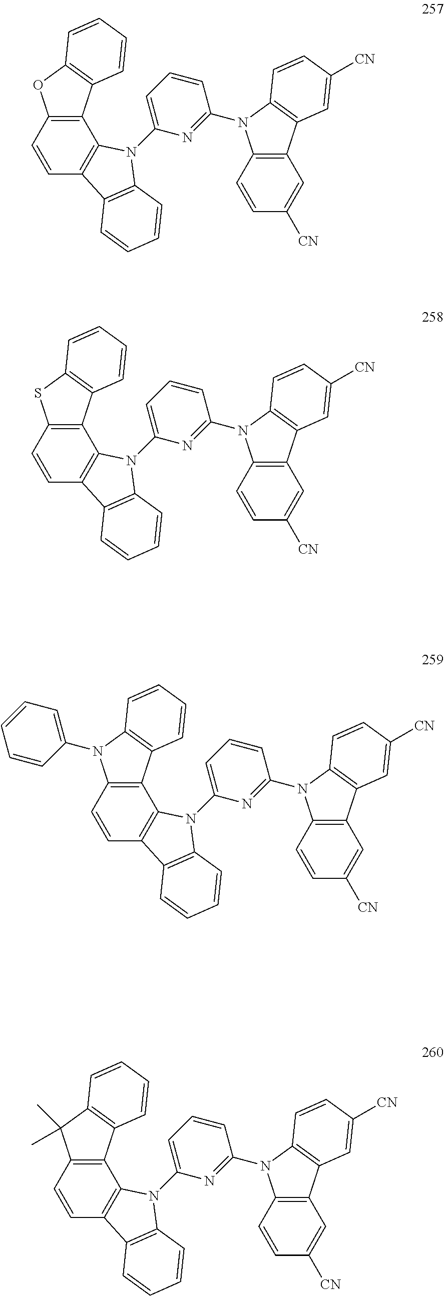 [SMILES: N#Cc1ccc2c(c1)c1cc(C#N)ccc1n2-c1cccc(-n2c3ccccc3c3ccc4oc5ccccc5c4c32)n1 ; N#Cc1ccc2c(c1)c1cc(C#N)ccc1n2-c1cccc(-n2c3ccccc3c3ccc4sc5ccccc5c4c32)n1 ; N#Cc1ccc2c(c1)c1cc(C#N)ccc1n2-c1cccc(-n2c3ccccc3c3ccc4c(c5ccccc5n4-c4ccccc4)c32)n1 ; CC1(C)c2ccccc2-c2c1ccc1c3ccccc3n(-c3cccc(-n4c5ccc(C#N)cc5c5cc(C#N)ccc54)n3)c21]

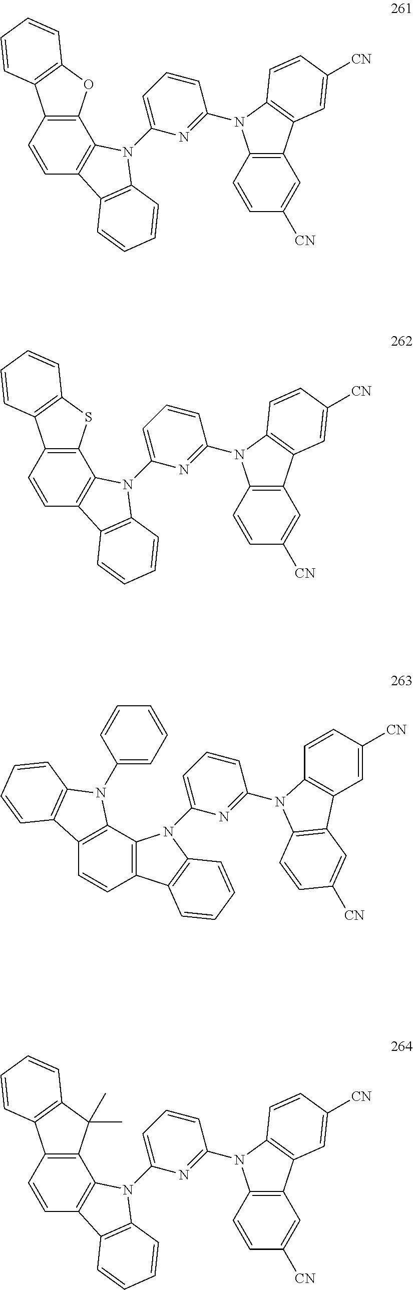 [SMILES: N#Cc1ccc2c(c1)c1cc(C#N)ccc1n2-c1cccc(-n2c3ccccc3c3ccc4c5ccccc5oc4c32)n1 ; N#Cc1ccc2c(c1)c1cc(C#N)ccc1n2-c1cccc(-n2c3ccccc3c3ccc4c5ccccc5sc4c32)n1 ; N#Cc1ccc2c(c1)c1cc(C#N)ccc1n2-c1cccc(-n2c3ccccc3c3ccc4c5ccccc5n(-c5ccccc5)c4c32)n1 ; CC1(C)c2ccccc2-c2ccc3c4ccccc4n(-c4cccc(-n5c6ccc(C#N)cc6c6cc(C#N)ccc65)n4)c3c21]

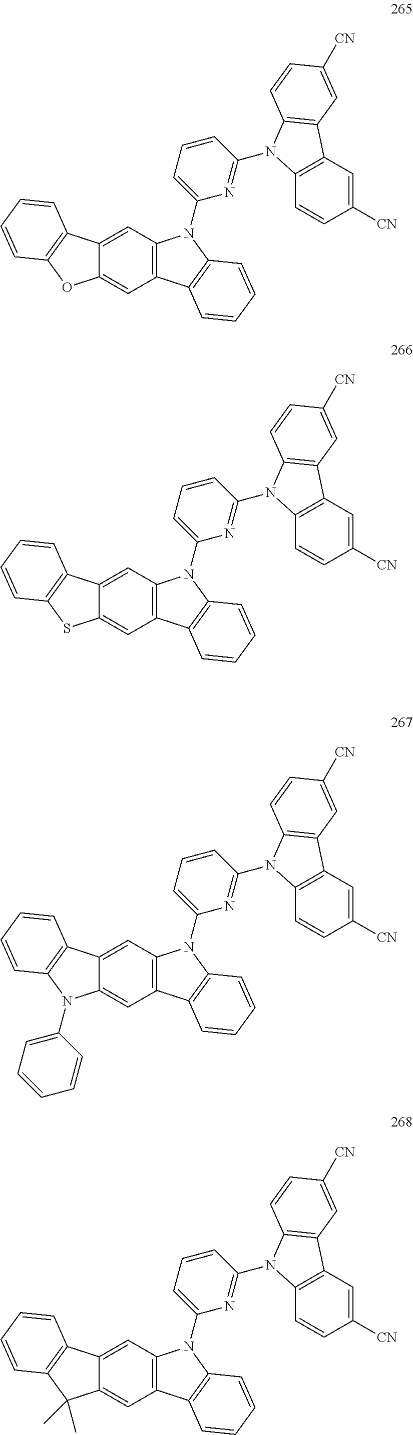 [SMILES: N#Cc1ccc2c(c1)c1cc(C#N)ccc1n2-c1cccc(-n2c3ccccc3c3cc4oc5ccccc5c4cc32)n1 ; N#Cc1ccc2c(c1)c1cc(C#N)ccc1n2-c1cccc(-n2c3ccccc3c3cc4sc5ccccc5c4cc32)n1 ; N#Cc1ccc2c(c1)c1cc(C#N)ccc1n2-c1cccc(-n2c3ccccc3c3cc4c(cc32)c2ccccc2n4-c2ccccc2)n1 ; CC1(C)c2ccccc2-c2cc3c(cc21)c1ccccc1n3-c1cccc(-n2c3ccc(C#N)cc3c3cc(C#N)ccc32)n1]

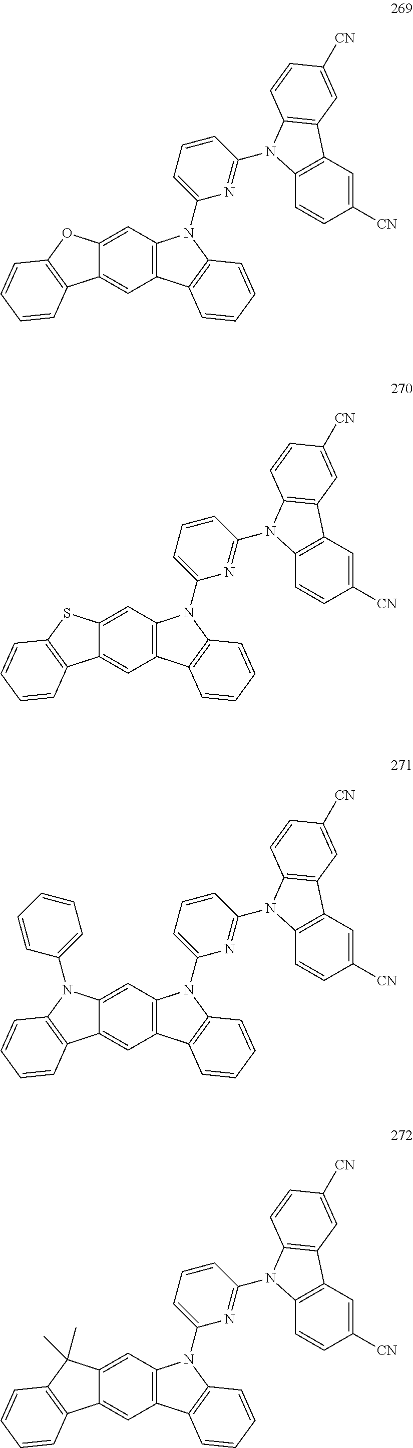 [SMILES: N#Cc1ccc2c(c1)c1cc(C#N)ccc1n2-c1cccc(-n2c3ccccc3c3cc4c(cc32)oc2ccccc24)n1 ; N#Cc1ccc2c(c1)c1cc(C#N)ccc1n2-c1cccc(-n2c3ccccc3c3cc4c(cc32)sc2ccccc24)n1 ; N#Cc1ccc2c(c1)c1cc(C#N)ccc1n2-c1cccc(-n2c3ccccc3c3cc4c5ccccc5n(-c5ccccc5)c4cc32)n1 ; CC1(C)c2ccccc2-c2cc3c4ccccc4n(-c4cccc(-n5c6ccc(C#N)cc6c6cc(C#N)ccc65)n4)c3cc21]

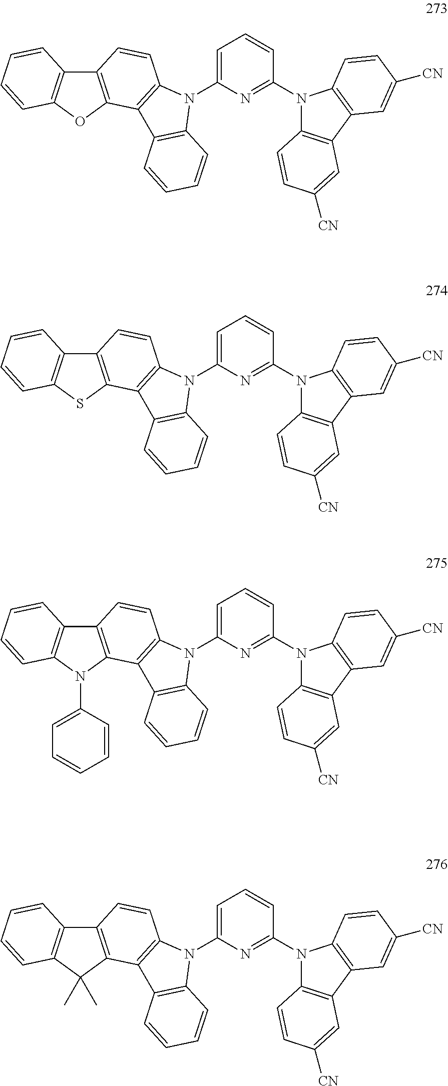 [SMILES: N#Cc1ccc2c(c1)c1cc(C#N)ccc1n2-c1cccc(-n2c3ccccc3c3c4oc5ccccc5c4ccc32)n1 ; N#Cc1ccc2c(c1)c1cc(C#N)ccc1n2-c1cccc(-n2c3ccccc3c3c4sc5ccccc5c4ccc32)n1 ; N#Cc1ccc2c(c1)c1cc(C#N)ccc1n2-c1cccc(-n2c3ccccc3c3c2ccc2c4ccccc4n(-c4ccccc4)c23)n1 ; CC1(C)c2ccccc2-c2ccc3c(c21)c1ccccc1n3-c1cccc(-n2c3ccc(C#N)cc3c3cc(C#N)ccc32)n1]

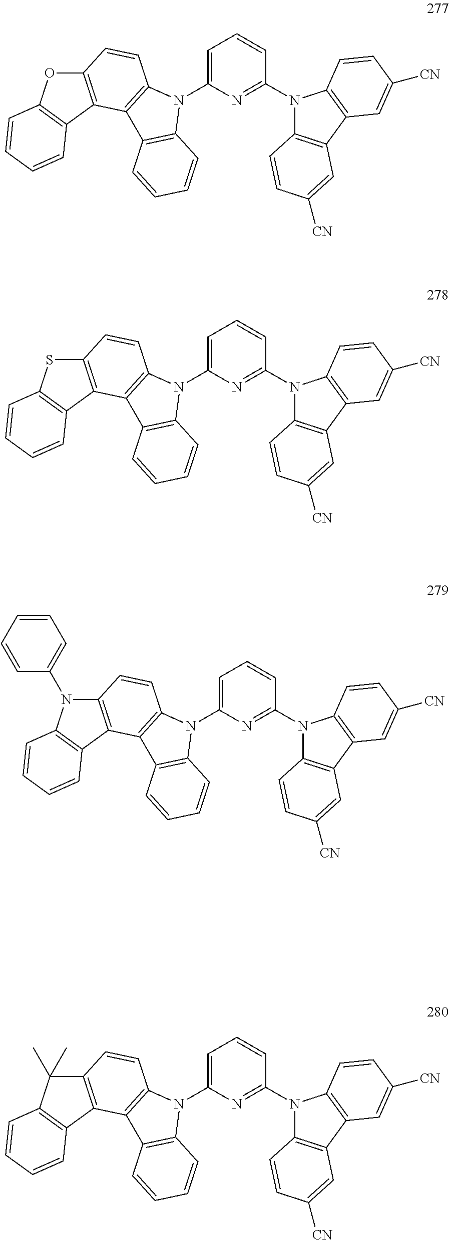 [SMILES: N#Cc1ccc2c(c1)c1cc(C#N)ccc1n2-c1cccc(-n2c3ccccc3c3c4c(ccc32)oc2ccccc24)n1 ; N#Cc1ccc2c(c1)c1cc(C#N)ccc1n2-c1cccc(-n2c3ccccc3c3c4c(ccc32)sc2ccccc24)n1 ; N#Cc1ccc2c(c1)c1cc(C#N)ccc1n2-c1cccc(-n2c3ccccc3c3c4c5ccccc5n(-c5ccccc5)c4ccc32)n1 ; CC1(C)c2ccccc2-c2c1ccc1c2c2ccccc2n1-c1cccc(-n2c3ccc(C#N)cc3c3cc(C#N)ccc32)n1]

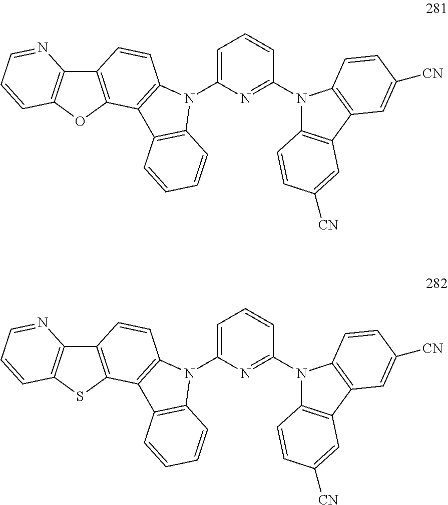 [SMILES: N#Cc1ccc2c(c1)c1cc(C#N)ccc1n2-c1cccc(-n2c3ccccc3c3c4oc5cccnc5c4ccc32)n1 ; N#Cc1ccc2c(c1)c1cc(C#N)ccc1n2-c1cccc(-n2c3ccccc3c3c4sc5cccnc5c4ccc32)n1]

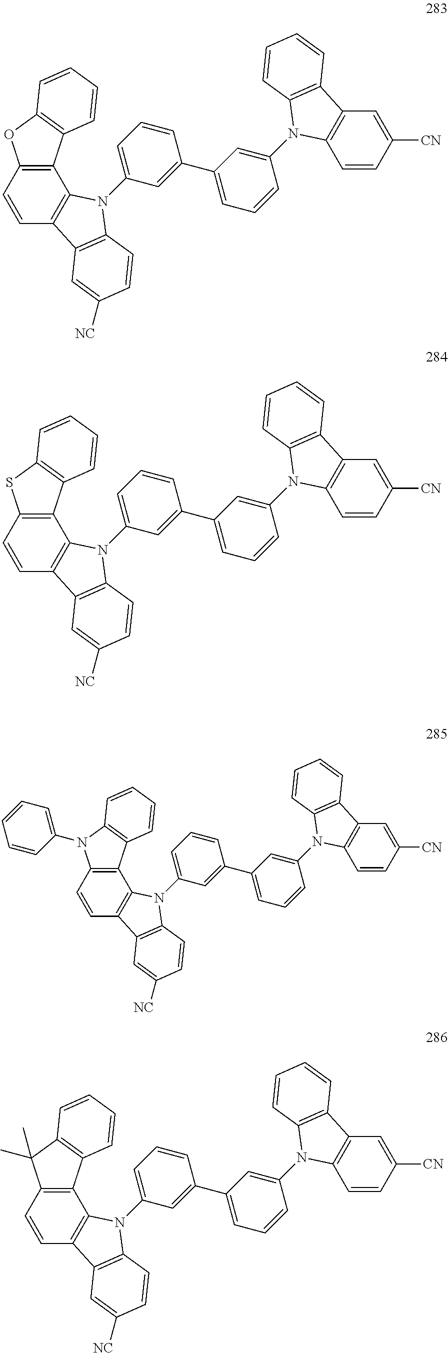 [SMILES: N#Cc1ccc2c(c1)c1ccccc1n2-c1cccc(-c2cccc(-n3c4ccc(C#N)cc4c4ccc5oc6ccccc6c5c43)c2)c1 ; N#Cc1ccc2c(c1)c1ccccc1n2-c1cccc(-c2cccc(-n3c4ccc(C#N)cc4c4ccc5sc6ccccc6c5c43)c2)c1 ; N#Cc1ccc2c(c1)c1ccccc1n2-c1cccc(-c2cccc(-n3c4ccc(C#N)cc4c4ccc5c(c6ccccc6n5-c5ccccc5)c43)c2)c1 ; CC1(C)c2ccccc2-c2c1ccc1c3cc(C#N)ccc3n(-c3cccc(-c4cccc(-n5c6ccccc6c6cc(C#N)ccc65)c4)c3)c21]

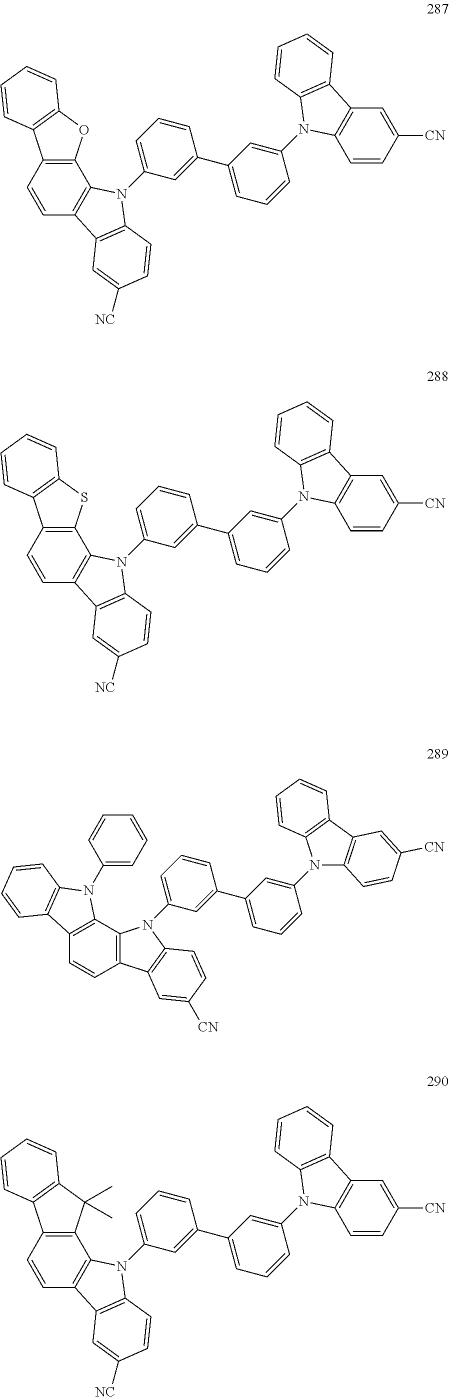 [SMILES: N#Cc1ccc2c(c1)c1ccccc1n2-c1cccc(-c2cccc(-n3c4ccc(C#N)cc4c4ccc5c6ccccc6oc5c43)c2)c1 ; N#Cc1ccc2c(c1)c1ccccc1n2-c1cccc(-c2cccc(-n3c4ccc(C#N)cc4c4ccc5c6ccccc6sc5c43)c2)c1 ; N#Cc1ccc2c(c1)c1ccccc1n2-c1cccc(-c2cccc(-n3c4ccc(C#N)cc4c4ccc5c6ccccc6n(-c6ccccc6)c5c43)c2)c1 ; CC1(C)c2ccccc2-c2ccc3c4cc(C#N)ccc4n(-c4cccc(-c5cccc(-n6c7ccccc7c7cc(C#N)ccc76)c5)c4)c3c21]

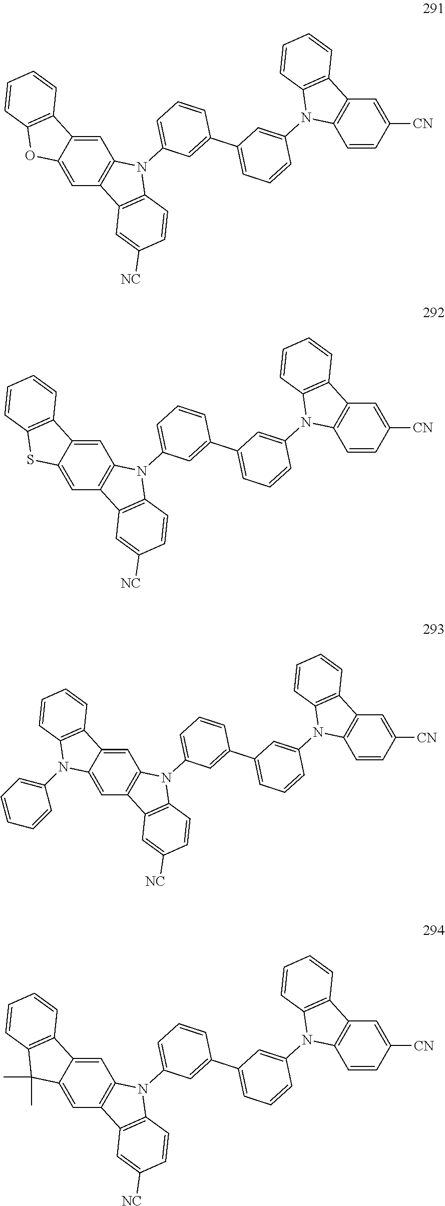 [SMILES: N#Cc1ccc2c(c1)c1ccccc1n2-c1cccc(-c2cccc(-n3c4ccc(C#N)cc4c4cc5oc6ccccc6c5cc43)c2)c1 ; N#Cc1ccc2c(c1)c1ccccc1n2-c1cccc(-c2cccc(-n3c4ccc(C#N)cc4c4cc5sc6ccccc6c5cc43)c2)c1 ; N#Cc1ccc2c(c1)c1ccccc1n2-c1cccc(-c2cccc(-n3c4ccc(C#N)cc4c4cc5c(cc43)c3ccccc3n5-c3ccccc3)c2)c1 ; CC1(C)c2ccccc2-c2cc3c(cc21)c1cc(C#N)ccc1n3-c1cccc(-c2cccc(-n3c4ccccc4c4cc(C#N)ccc43)c2)c1]

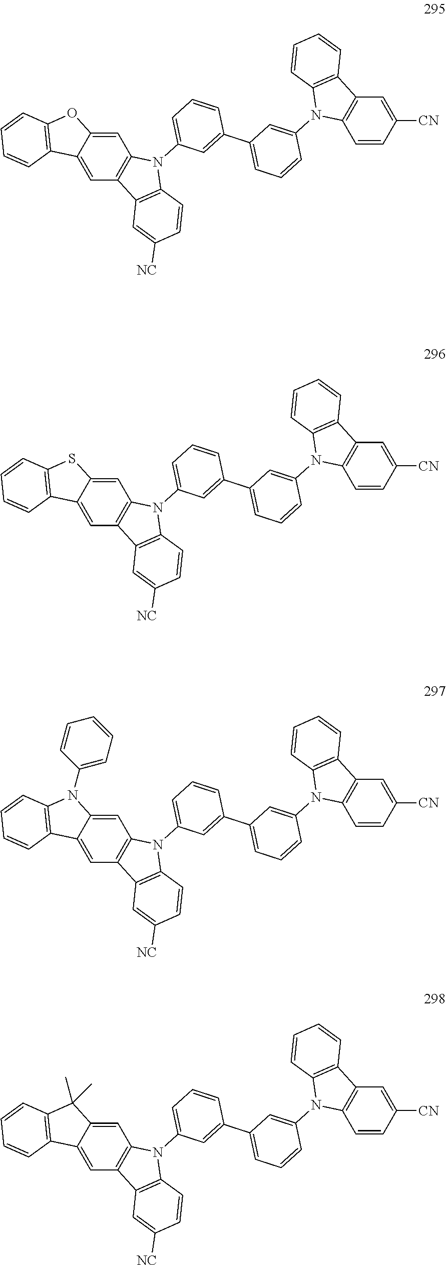 [SMILES: N#Cc1ccc2c(c1)c1ccccc1n2-c1cccc(-c2cccc(-n3c4ccc(C#N)cc4c4cc5c(cc43)oc3ccccc35)c2)c1 ; N#Cc1ccc2c(c1)c1ccccc1n2-c1cccc(-c2cccc(-n3c4ccc(C#N)cc4c4cc5c(cc43)sc3ccccc35)c2)c1 ; N#Cc1ccc2c(c1)c1ccccc1n2-c1cccc(-c2cccc(-n3c4ccc(C#N)cc4c4cc5c6ccccc6n(-c6ccccc6)c5cc43)c2)c1 ; CC1(C)c2ccccc2-c2cc3c4cc(C#N)ccc4n(-c4cccc(-c5cccc(-n6c7ccccc7c7cc(C#N)ccc76)c5)c4)c3cc21]

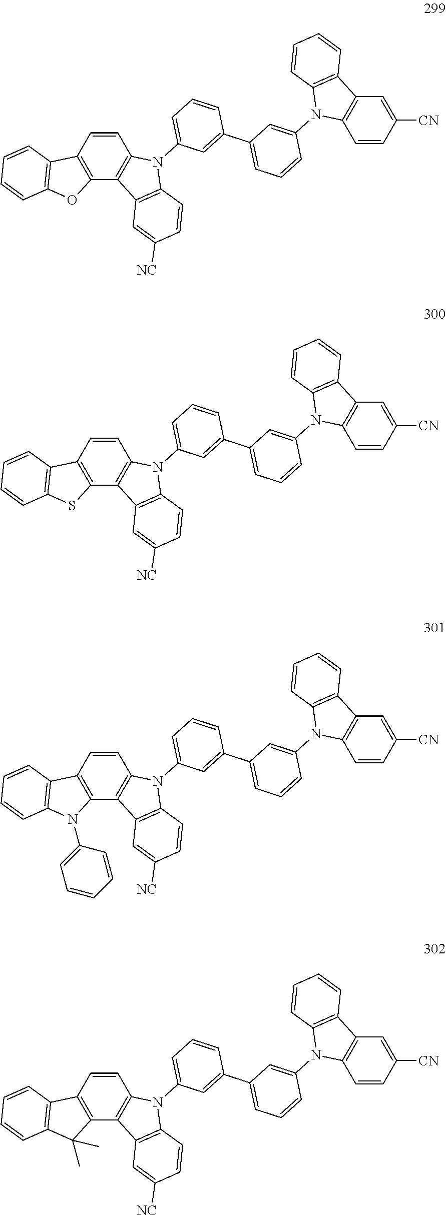 [SMILES: N#Cc1ccc2c(c1)c1ccccc1n2-c1cccc(-c2cccc(-n3c4ccc(C#N)cc4c4c5oc6ccccc6c5ccc43)c2)c1 ; N#Cc1ccc2c(c1)c1ccccc1n2-c1cccc(-c2cccc(-n3c4ccc(C#N)cc4c4c5sc6ccccc6c5ccc43)c2)c1 ; N#Cc1ccc2c(c1)c1ccccc1n2-c1cccc(-c2cccc(-n3c4ccc(C#N)cc4c4c3ccc3c5ccccc5n(-c5ccccc5)c34)c2)c1 ; CC1(C)c2ccccc2-c2ccc3c(c21)c1cc(C#N)ccc1n3-c1cccc(-c2cccc(-n3c4ccccc4c4cc(C#N)ccc43)c2)c1]

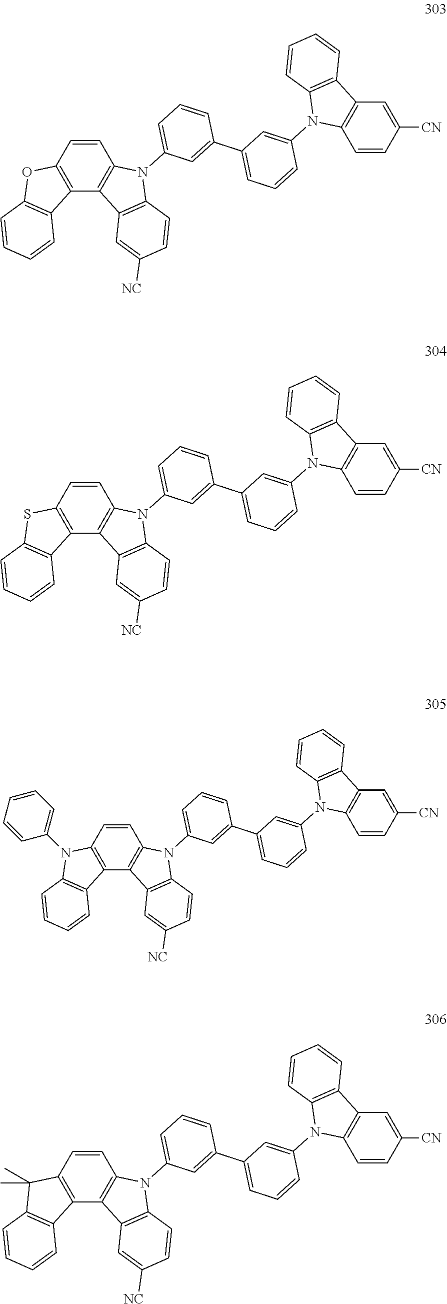 [SMILES: N#Cc1ccc2c(c1)c1ccccc1n2-c1cccc(-c2cccc(-n3c4ccc(C#N)cc4c4c5c(ccc43)oc3ccccc35)c2)c1 ; N#Cc1ccc2c(c1)c1ccccc1n2-c1cccc(-c2cccc(-n3c4ccc(C#N)cc4c4c5c(ccc43)sc3ccccc35)c2)c1 ; N#Cc1ccc2c(c1)c1ccccc1n2-c1cccc(-c2cccc(-n3c4ccc(C#N)cc4c4c5c6ccccc6n(-c6ccccc6)c5ccc43)c2)c1 ; CC1(C)c2ccccc2-c2c1ccc1c2c2cc(C#N)ccc2n1-c1cccc(-c2cccc(-n3c4ccccc4c4cc(C#N)ccc43)c2)c1]

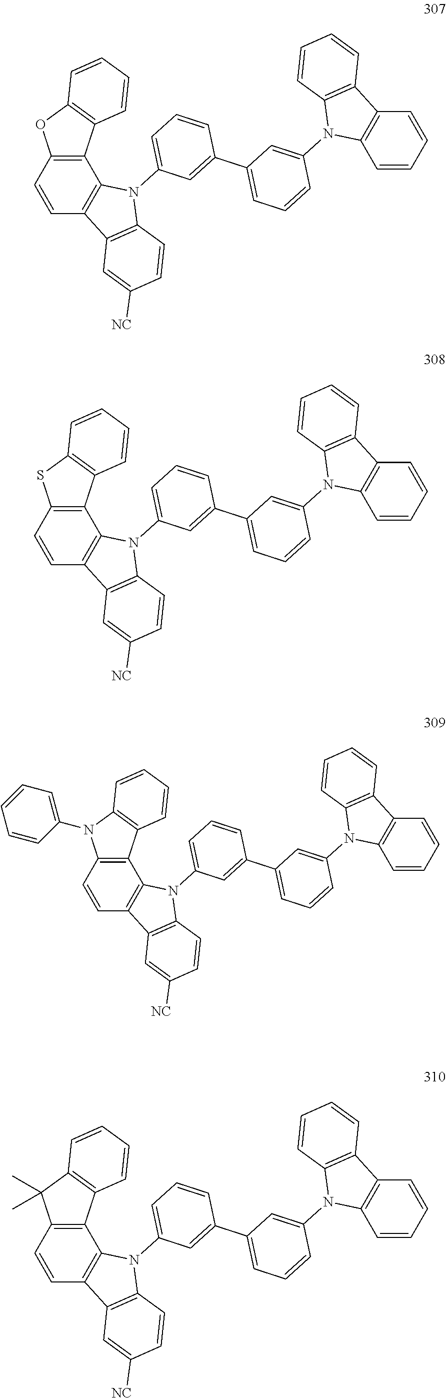 [SMILES: N#Cc1ccc2c(c1)c1ccc3oc4ccccc4c3c1n2-c1cccc(-c2cccc(-n3c4ccccc4c4ccccc43)c2)c1 ; N#Cc1ccc2c(c1)c1ccc3sc4ccccc4c3c1n2-c1cccc(-c2cccc(-n3c4ccccc4c4ccccc43)c2)c1 ; N#Cc1ccc2c(c1)c1ccc3c(c4ccccc4n3-c3ccccc3)c1n2-c1cccc(-c2cccc(-n3c4ccccc4c4ccccc43)c2)c1 ; CC1(C)c2ccccc2-c2c1ccc1c3cc(C#N)ccc3n(-c3cccc(-c4cccc(-n5c6ccccc6c6ccccc65)c4)c3)c21]

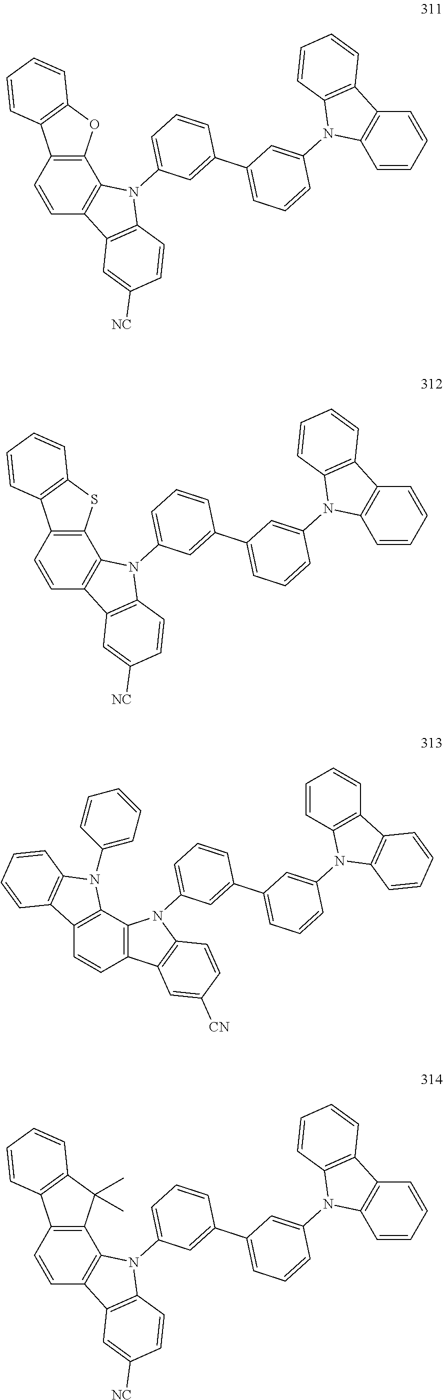 [SMILES: N#Cc1ccc2c(c1)c1ccc3c4ccccc4oc3c1n2-c1cccc(-c2cccc(-n3c4ccccc4c4ccccc43)c2)c1 ; N#Cc1ccc2c(c1)c1ccc3c4ccccc4sc3c1n2-c1cccc(-c2cccc(-n3c4ccccc4c4ccccc43)c2)c1 ; N#Cc1ccc2c(c1)c1ccc3c4ccccc4n(-c4ccccc4)c3c1n2-c1cccc(-c2cccc(-n3c4ccccc4c4ccccc43)c2)c1 ; CC1(C)c2ccccc2-c2ccc3c4cc(C#N)ccc4n(-c4cccc(-c5cccc(-n6c7ccccc7c7ccccc76)c5)c4)c3c21]

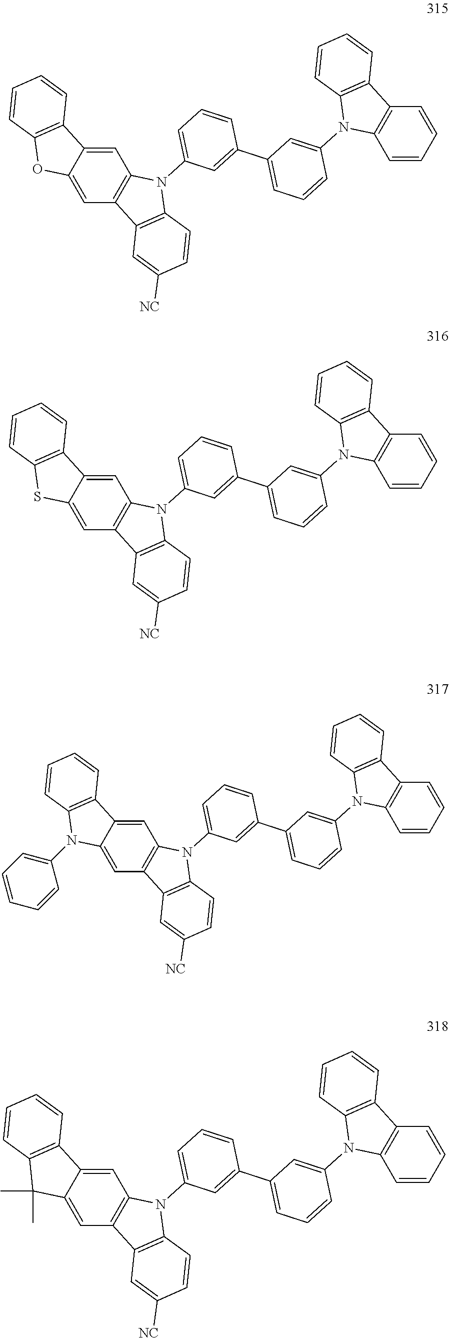 [SMILES: N#Cc1ccc2c(c1)c1cc3oc4ccccc4c3cc1n2-c1cccc(-c2cccc(-n3c4ccccc4c4ccccc43)c2)c1 ; N#Cc1ccc2c(c1)c1cc3sc4ccccc4c3cc1n2-c1cccc(-c2cccc(-n3c4ccccc4c4ccccc43)c2)c1 ; N#Cc1ccc2c(c1)c1cc3c(cc1n2-c1cccc(-c2cccc(-n4c5ccccc5c5ccccc54)c2)c1)c1ccccc1n3-c1ccccc1 ; CC1(C)c2ccccc2-c2cc3c(cc21)c1cc(C#N)ccc1n3-c1cccc(-c2cccc(-n3c4ccccc4c4ccccc43)c2)c1]

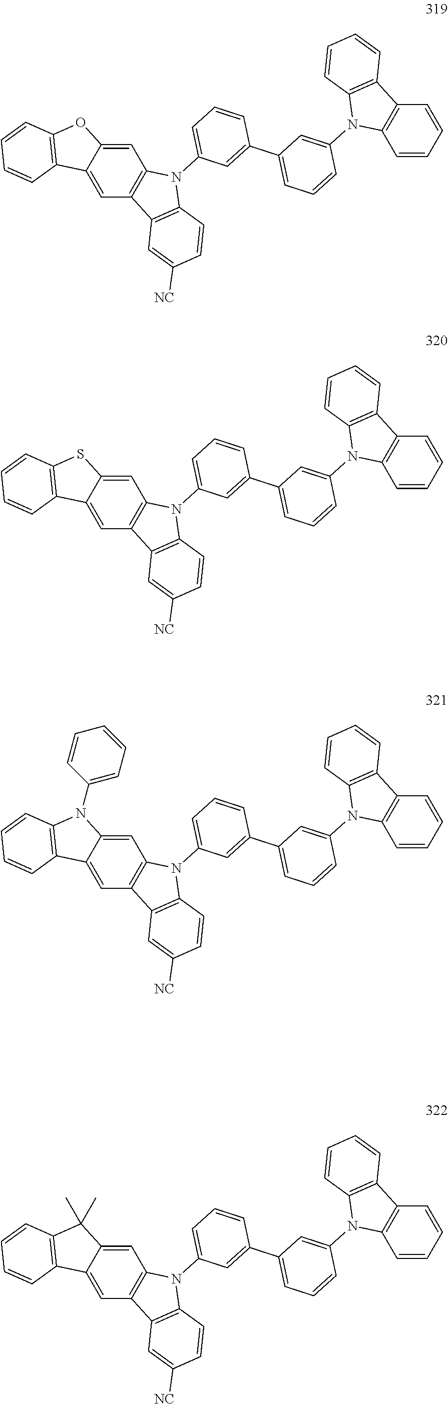 [SMILES: N#Cc1ccc2c(c1)c1cc3c(cc1n2-c1cccc(-c2cccc(-n4c5ccccc5c5ccccc54)c2)c1)oc1ccccc13 ; N#Cc1ccc2c(c1)c1cc3c(cc1n2-c1cccc(-c2cccc(-n4c5ccccc5c5ccccc54)c2)c1)sc1ccccc13 ; N#Cc1ccc2c(c1)c1cc3c4ccccc4n(-c4ccccc4)c3cc1n2-c1cccc(-c2cccc(-n3c4ccccc4c4ccccc43)c2)c1 ; CC1(C)c2ccccc2-c2cc3c4cc(C#N)ccc4n(-c4cccc(-c5cccc(-n6c7ccccc7c7ccccc76)c5)c4)c3cc21]

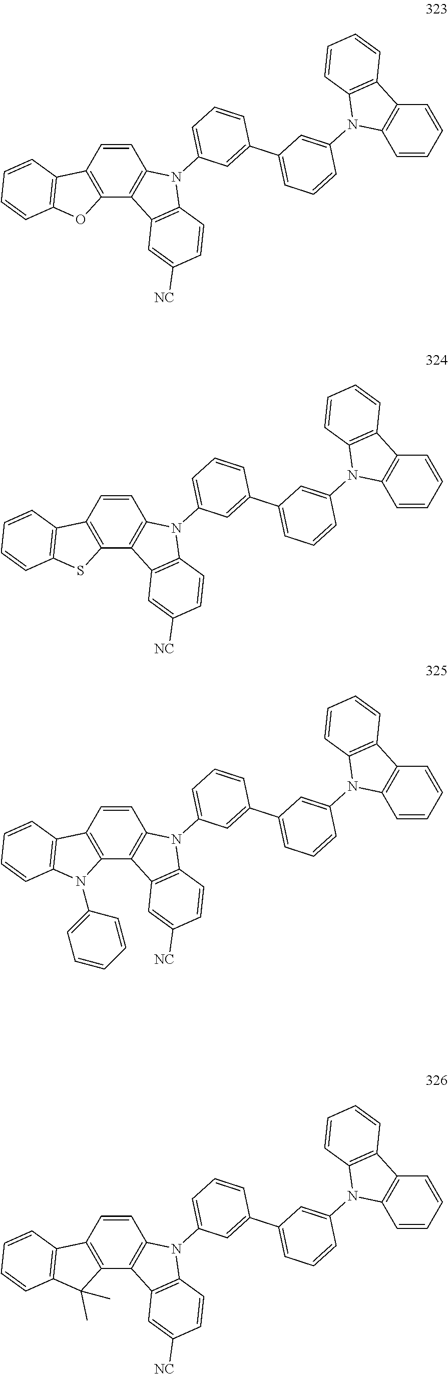 [SMILES: N#Cc1ccc2c(c1)c1c3oc4ccccc4c3ccc1n2-c1cccc(-c2cccc(-n3c4ccccc4c4ccccc43)c2)c1 ; N#Cc1ccc2c(c1)c1c3sc4ccccc4c3ccc1n2-c1cccc(-c2cccc(-n3c4ccccc4c4ccccc43)c2)c1 ; N#Cc1ccc2c(c1)c1c(ccc3c4ccccc4n(-c4ccccc4)c31)n2-c1cccc(-c2cccc(-n3c4ccccc4c4ccccc43)c2)c1 ; CC1(C)c2ccccc2-c2ccc3c(c21)c1cc(C#N)ccc1n3-c1cccc(-c2cccc(-n3c4ccccc4c4ccccc43)c2)c1]

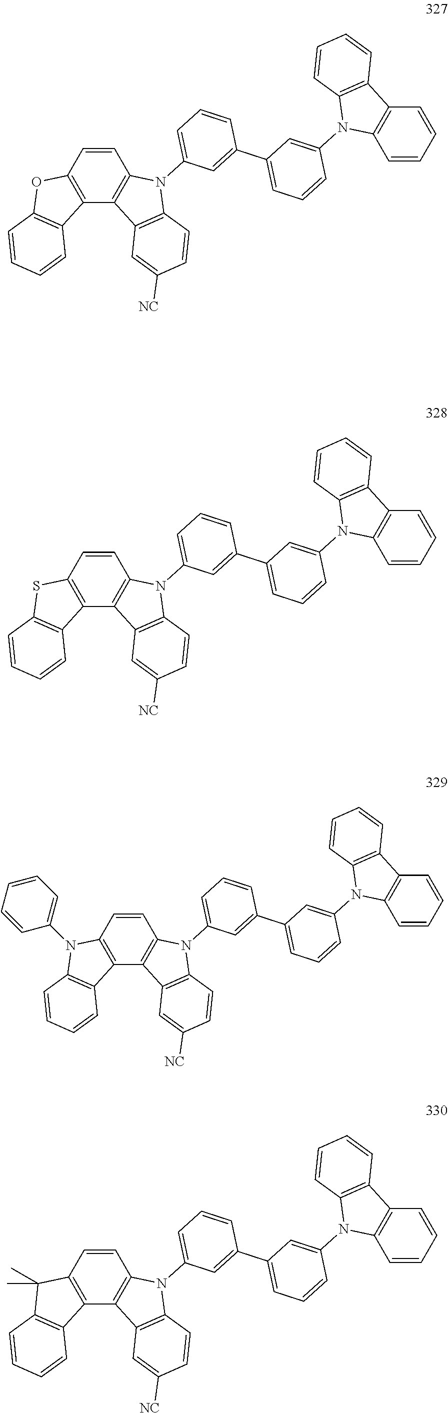 [SMILES: N#Cc1ccc2c(c1)c1c3c(ccc1n2-c1cccc(-c2cccc(-n4c5ccccc5c5ccccc54)c2)c1)oc1ccccc13 ; N#Cc1ccc2c(c1)c1c3c(ccc1n2-c1cccc(-c2cccc(-n4c5ccccc5c5ccccc54)c2)c1)sc1ccccc13 ; N#Cc1ccc2c(c1)c1c3c4ccccc4n(-c4ccccc4)c3ccc1n2-c1cccc(-c2cccc(-n3c4ccccc4c4ccccc43)c2)c1 ; CC1(C)c2ccccc2-c2c1ccc1c2c2cc(C#N)ccc2n1-c1cccc(-c2cccc(-n3c4ccccc4c4ccccc43)c2)c1]

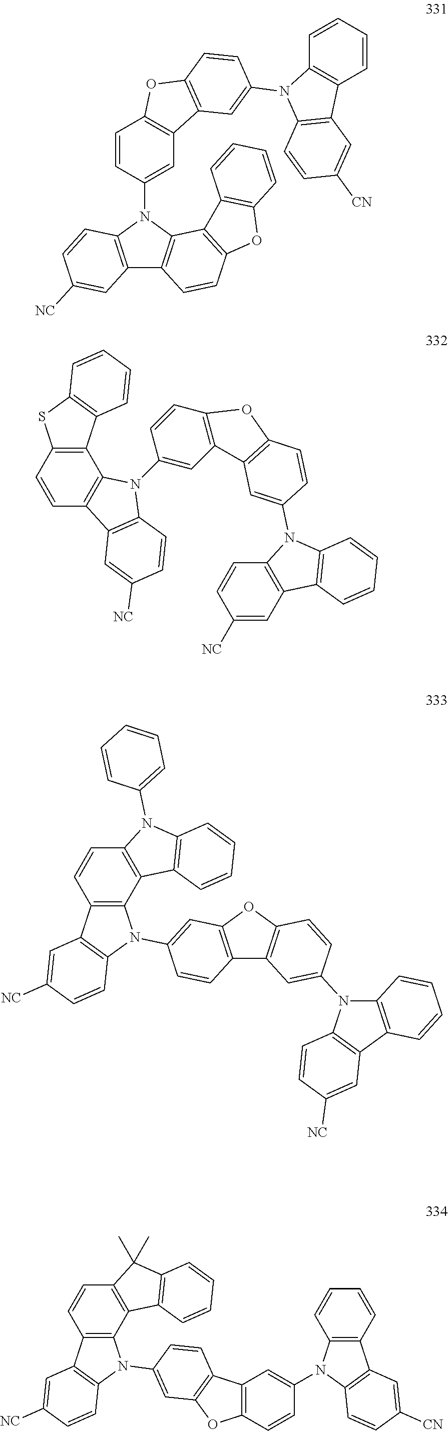 [SMILES: N#Cc1ccc2c(c1)c1ccccc1n2-c1ccc2oc3ccc(-n4c5ccc(C#N)cc5c5ccc6oc7ccccc7c6c54)cc3c2c1 ; N#Cc1ccc2c(c1)c1ccccc1n2-c1ccc2oc3ccc(-n4c5ccc(C#N)cc5c5ccc6sc7ccccc7c6c54)cc3c2c1 ; N#Cc1ccc2c(c1)c1ccccc1n2-c1ccc2oc3cc(-n4c5ccc(C#N)cc5c5ccc6c(c7ccccc7n6-c6ccccc6)c54)ccc3c2c1 ; CC1(C)c2ccccc2-c2c1ccc1c3cc(C#N)ccc3n(-c3ccc4c(c3)oc3ccc(-n5c6ccccc6c6cc(C#N)ccc65)cc34)c21]

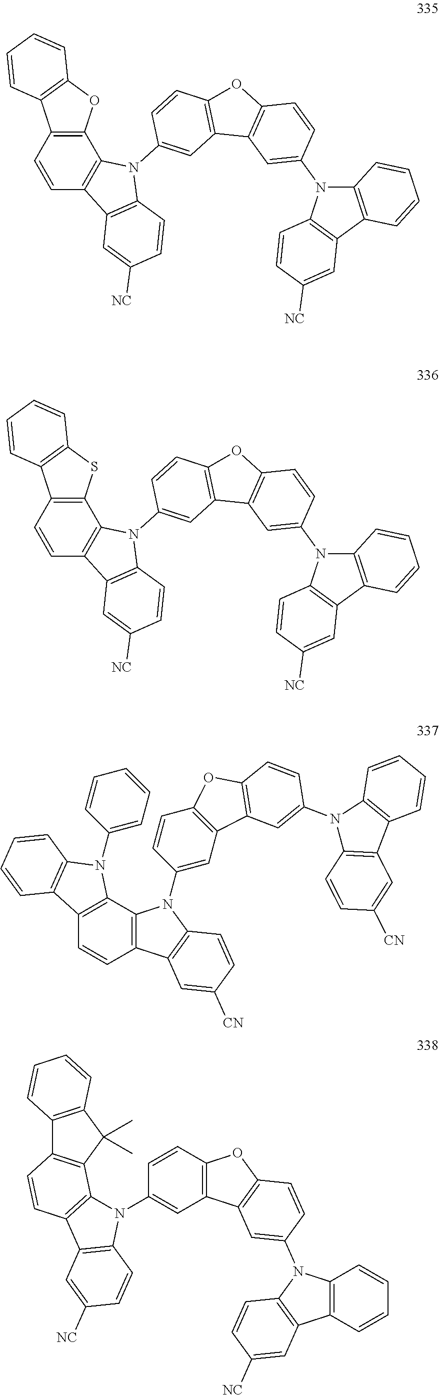 [SMILES: N#Cc1ccc2c(c1)c1ccccc1n2-c1ccc2oc3ccc(-n4c5ccc(C#N)cc5c5ccc6c7ccccc7oc6c54)cc3c2c1 ; N#Cc1ccc2c(c1)c1ccccc1n2-c1ccc2oc3ccc(-n4c5ccc(C#N)cc5c5ccc6c7ccccc7sc6c54)cc3c2c1 ; N#Cc1ccc2c(c1)c1ccccc1n2-c1ccc2oc3ccc(-n4c5ccc(C#N)cc5c5ccc6c7ccccc7n(-c7ccccc7)c6c54)cc3c2c1 ; CC1(C)c2ccccc2-c2ccc3c4cc(C#N)ccc4n(-c4ccc5oc6ccc(-n7c8ccccc8c8cc(C#N)ccc87)cc6c5c4)c3c21]

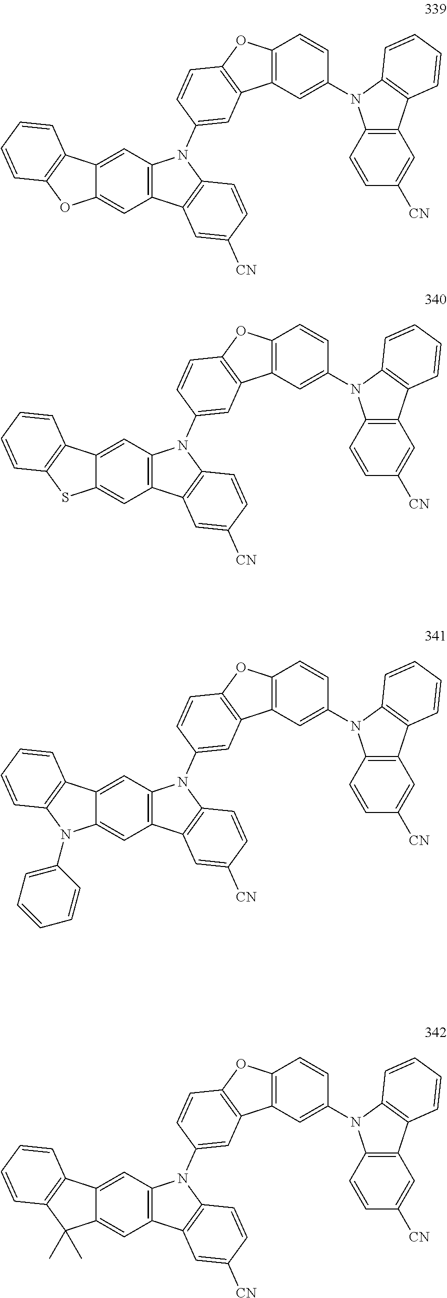 [SMILES: N#Cc1ccc2c(c1)c1ccccc1n2-c1ccc2oc3ccc(-n4c5ccc(C#N)cc5c5cc6oc7ccccc7c6cc54)cc3c2c1 ; N#Cc1ccc2c(c1)c1ccccc1n2-c1ccc2oc3ccc(-n4c5ccc(C#N)cc5c5cc6sc7ccccc7c6cc54)cc3c2c1 ; N#Cc1ccc2c(c1)c1ccccc1n2-c1ccc2oc3ccc(-n4c5ccc(C#N)cc5c5cc6c(cc54)c4ccccc4n6-c4ccccc4)cc3c2c1 ; CC1(C)c2ccccc2-c2cc3c(cc21)c1cc(C#N)ccc1n3-c1ccc2oc3ccc(-n4c5ccccc5c5cc(C#N)ccc54)cc3c2c1]

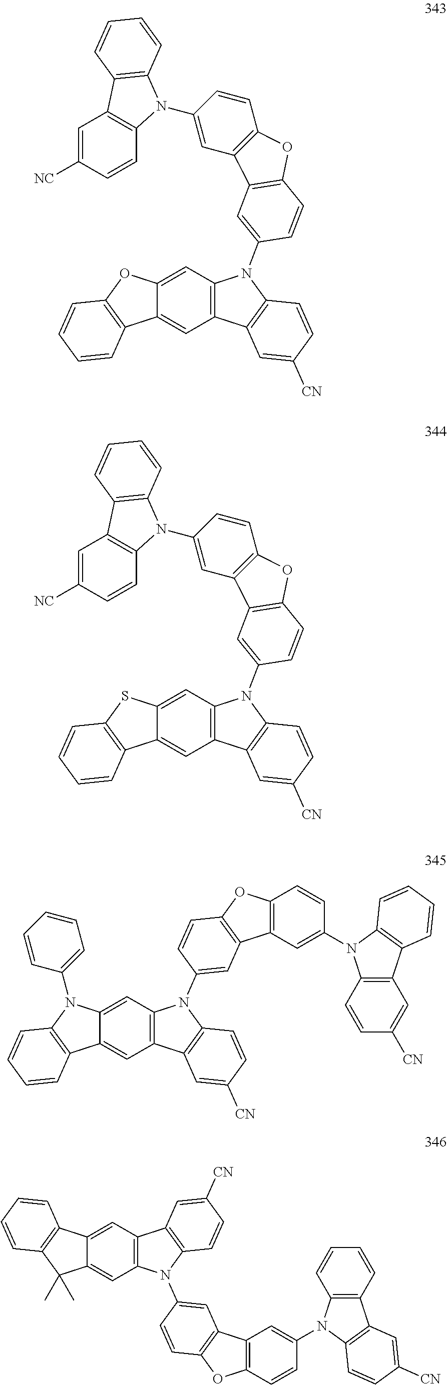 [SMILES: N#Cc1ccc2c(c1)c1ccccc1n2-c1ccc2oc3ccc(-n4c5ccc(C#N)cc5c5cc6c(cc54)oc4ccccc46)cc3c2c1 ; N#Cc1ccc2c(c1)c1ccccc1n2-c1ccc2oc3ccc(-n4c5ccc(C#N)cc5c5cc6c(cc54)sc4ccccc46)cc3c2c1 ; N#Cc1ccc2c(c1)c1ccccc1n2-c1ccc2oc3ccc(-n4c5ccc(C#N)cc5c5cc6c7ccccc7n(-c7ccccc7)c6cc54)cc3c2c1 ; CC1(C)c2ccccc2-c2cc3c4cc(C#N)ccc4n(-c4ccc5oc6ccc(-n7c8ccccc8c8cc(C#N)ccc87)cc6c5c4)c3cc21]

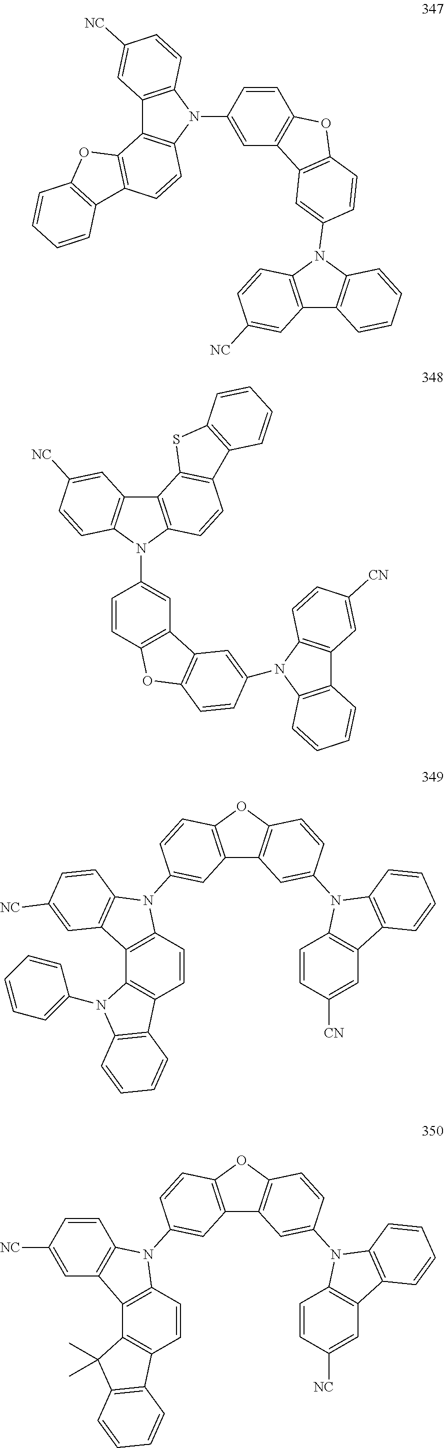

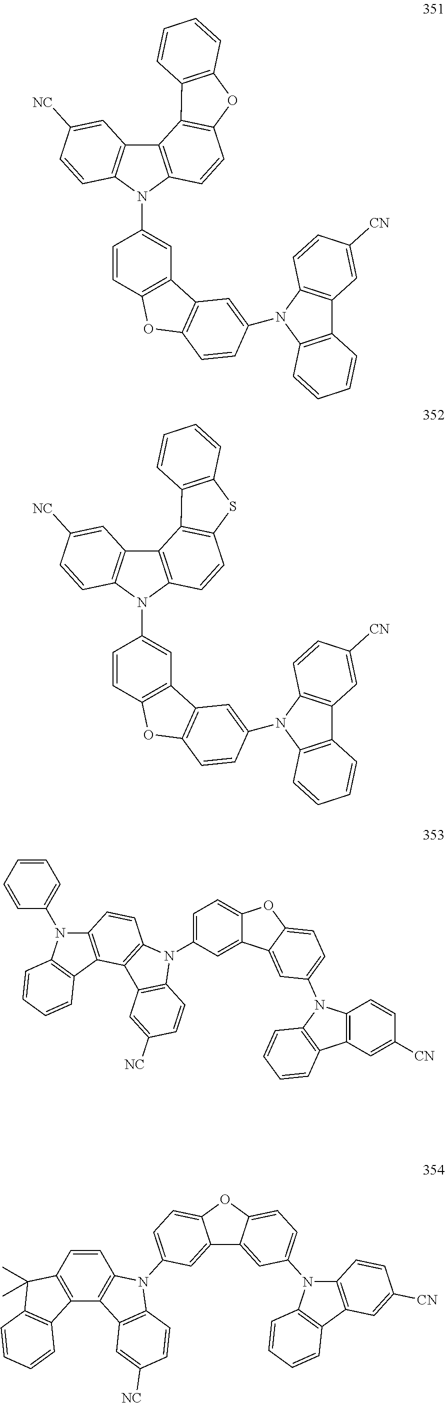 [SMILES: N#Cc1ccc2c(c1)c1ccccc1n2-c1ccc2oc3ccc(-n4c5ccc(C#N)cc5c5c6c(ccc54)oc4ccccc46)cc3c2c1 ; N#Cc1ccc2c(c1)c1ccccc1n2-c1ccc2oc3ccc(-n4c5ccc(C#N)cc5c5c6c(ccc54)sc4ccccc46)cc3c2c1 ; N#Cc1ccc2c(c1)c1ccccc1n2-c1ccc2oc3ccc(-n4c5ccc(C#N)cc5c5c6c7ccccc7n(-c7ccccc7)c6ccc54)cc3c2c1 ; CC1(C)c2ccccc2-c2c1ccc1c2c2cc(C#N)ccc2n1-c1ccc2oc3ccc(-n4c5ccccc5c5cc(C#N)ccc54)cc3c2c1]

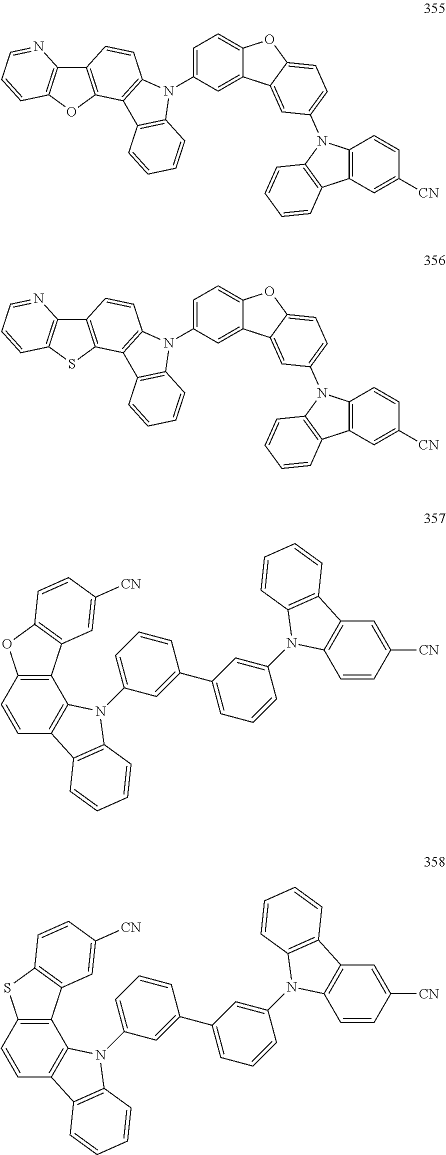 [SMILES: N#Cc1ccc2c(c1)c1ccccc1n2-c1ccc2oc3ccc(-n4c5ccccc5c5c6oc7cccnc7c6ccc54)cc3c2c1 ; N#Cc1ccc2c(c1)c1ccccc1n2-c1ccc2oc3ccc(-n4c5ccccc5c5c6sc7cccnc7c6ccc54)cc3c2c1 ; N#Cc1ccc2oc3ccc4c5ccccc5n(-c5cccc(-c6cccc(-n7c8ccccc8c8cc(C#N)ccc87)c6)c5)c4c3c2c1 ; N#Cc1ccc2sc3ccc4c5ccccc5n(-c5cccc(-c6cccc(-n7c8ccccc8c8cc(C#N)ccc87)c6)c5)c4c3c2c1]

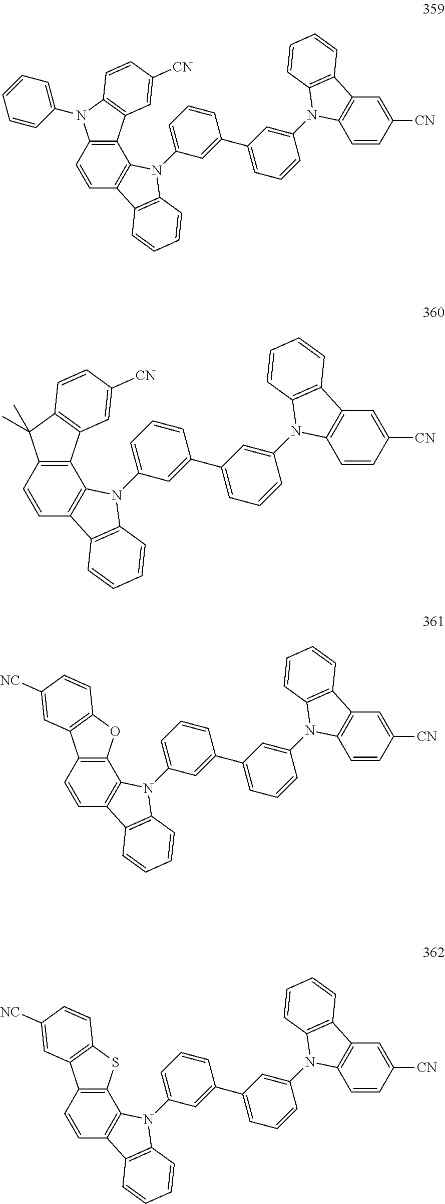 [SMILES: N#Cc1ccc2c(c1)c1ccccc1n2-c1cccc(-c2cccc(-n3c4ccccc4c4ccc5c(c6cc(C#N)ccc6n5-c5ccccc5)c43)c2)c1 ; CC1(C)c2ccc(C#N)cc2-c2c1ccc1c3ccccc3n(-c3cccc(-c4cccc(-n5c6ccccc6c6cc(C#N)ccc65)c4)c3)c21 ; N#Cc1ccc2oc3c(ccc4c5ccccc5n(-c5cccc(-c6cccc(-n7c8ccccc8c8cc(C#N)ccc87)c6)c5)c43)c2c1 ; N#Cc1ccc2sc3c(ccc4c5ccccc5n(-c5cccc(-c6cccc(-n7c8ccccc8c8cc(C#N)ccc87)c6)c5)c43)c2c1]

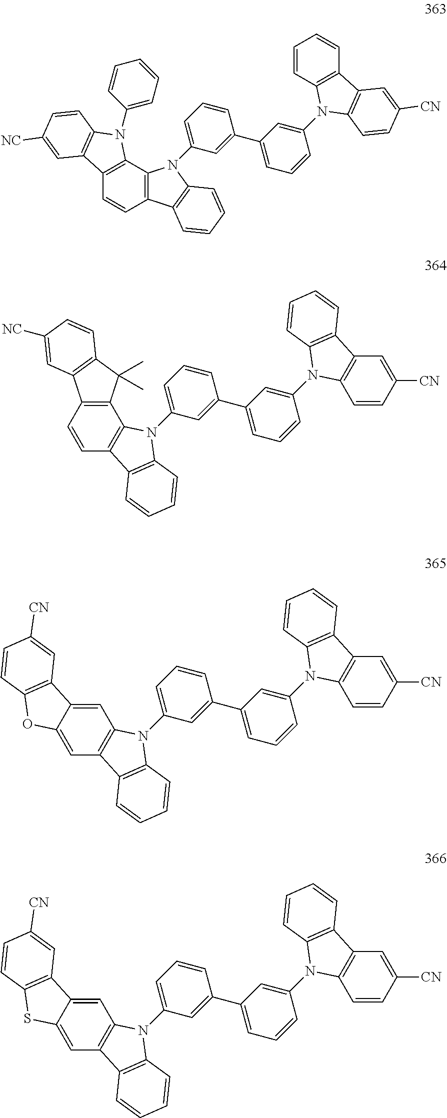 [SMILES: N#Cc1ccc2c(c1)c1ccccc1n2-c1cccc(-c2cccc(-n3c4ccccc4c4ccc5c6cc(C#N)ccc6n(-c6ccccc6)c5c43)c2)c1 ; CC1(C)c2ccc(C#N)cc2-c2ccc3c4ccccc4n(-c4cccc(-c5cccc(-n6c7ccccc7c7cc(C#N)ccc76)c5)c4)c3c21 ; N#Cc1ccc2oc3cc4c5ccccc5n(-c5cccc(-c6cccc(-n7c8ccccc8c8cc(C#N)ccc87)c6)c5)c4cc3c2c1 ; N#Cc1ccc2sc3cc4c5ccccc5n(-c5cccc(-c6cccc(-n7c8ccccc8c8cc(C#N)ccc87)c6)c5)c4cc3c2c1]

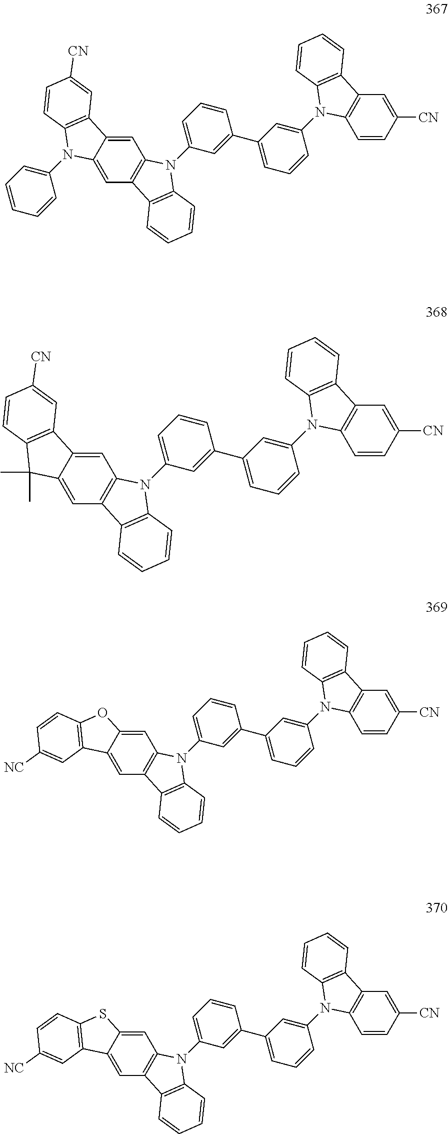 [SMILES: N#Cc1ccc2c(c1)c1ccccc1n2-c1cccc(-c2cccc(-n3c4ccccc4c4cc5c(cc43)c3cc(C#N)ccc3n5-c3ccccc3)c2)c1 ; CC1(C)c2ccc(C#N)cc2-c2cc3c(cc21)c1ccccc1n3-c1cccc(-c2cccc(-n3c4ccccc4c4cc(C#N)ccc43)c2)c1 ; N#Cc1ccc2oc3cc4c(cc3c2c1)c1ccccc1n4-c1cccc(-c2cccc(-n3c4ccccc4c4cc(C#N)ccc43)c2)c1 ; N#Cc1ccc2sc3cc4c(cc3c2c1)c1ccccc1n4-c1cccc(-c2cccc(-n3c4ccccc4c4cc(C#N)ccc43)c2)c1]

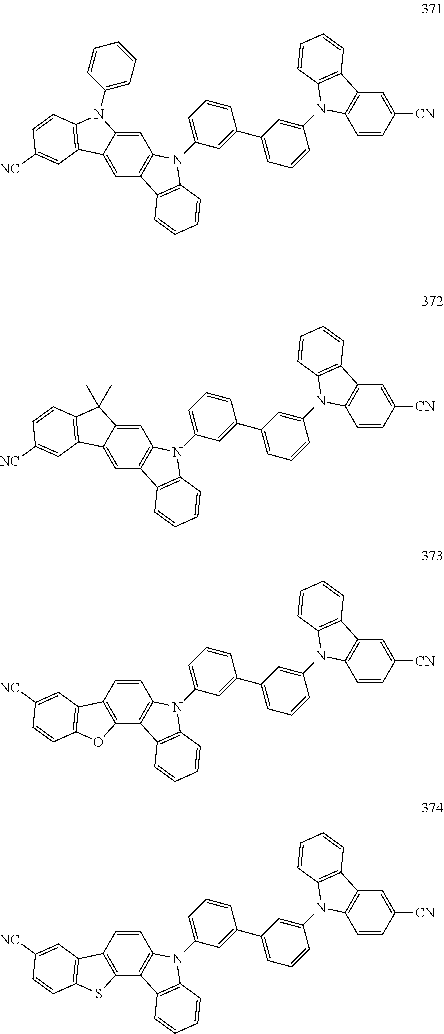 [SMILES: N#Cc1ccc2c(c1)c1ccccc1n2-c1cccc(-c2cccc(-n3c4ccccc4c4cc5c6cc(C#N)ccc6n(-c6ccccc6)c5cc43)c2)c1 ; CC1(C)c2ccc(C#N)cc2-c2cc3c4ccccc4n(-c4cccc(-c5cccc(-n6c7ccccc7c7cc(C#N)ccc76)c5)c4)c3cc21 ; N#Cc1ccc2oc3c(ccc4c3c3ccccc3n4-c3cccc(-c4cccc(-n5c6ccccc6c6cc(C#N)ccc65)c4)c3)c2c1 ; N#Cc1ccc2sc3c(ccc4c3c3ccccc3n4-c3cccc(-c4cccc(-n5c6ccccc6c6cc(C#N)ccc65)c4)c3)c2c1]

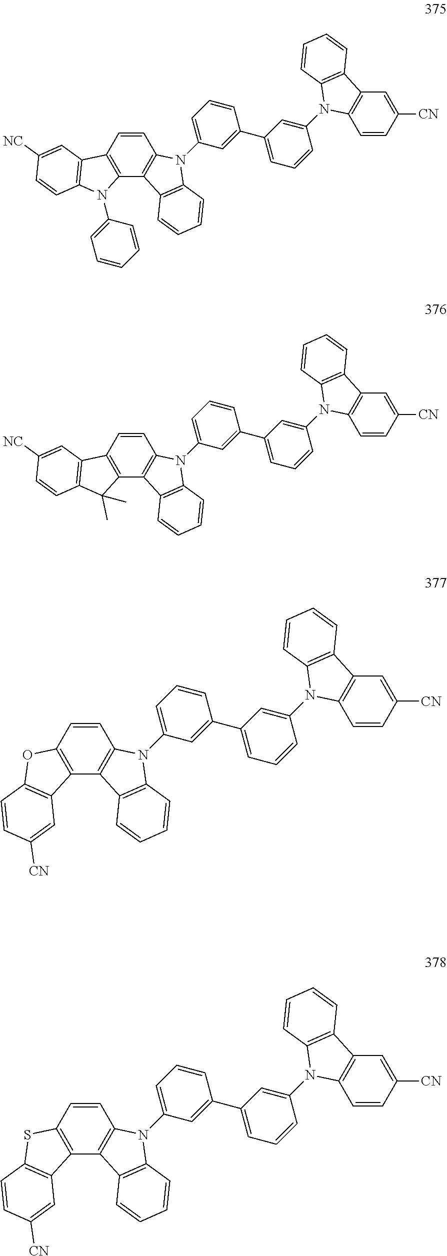 [SMILES: N#Cc1ccc2c(c1)c1ccccc1n2-c1cccc(-c2cccc(-n3c4ccccc4c4c3ccc3c5cc(C#N)ccc5n(-c5ccccc5)c34)c2)c1 ; CC1(C)c2ccc(C#N)cc2-c2ccc3c(c21)c1ccccc1n3-c1cccc(-c2cccc(-n3c4ccccc4c4cc(C#N)ccc43)c2)c1 ; N#Cc1ccc2oc3ccc4c(c5ccccc5n4-c4cccc(-c5cccc(-n6c7ccccc7c7cc(C#N)ccc76)c5)c4)c3c2c1 ; N#Cc1ccc2sc3ccc4c(c5ccccc5n4-c4cccc(-c5cccc(-n6c7ccccc7c7cc(C#N)ccc76)c5)c4)c3c2c1]

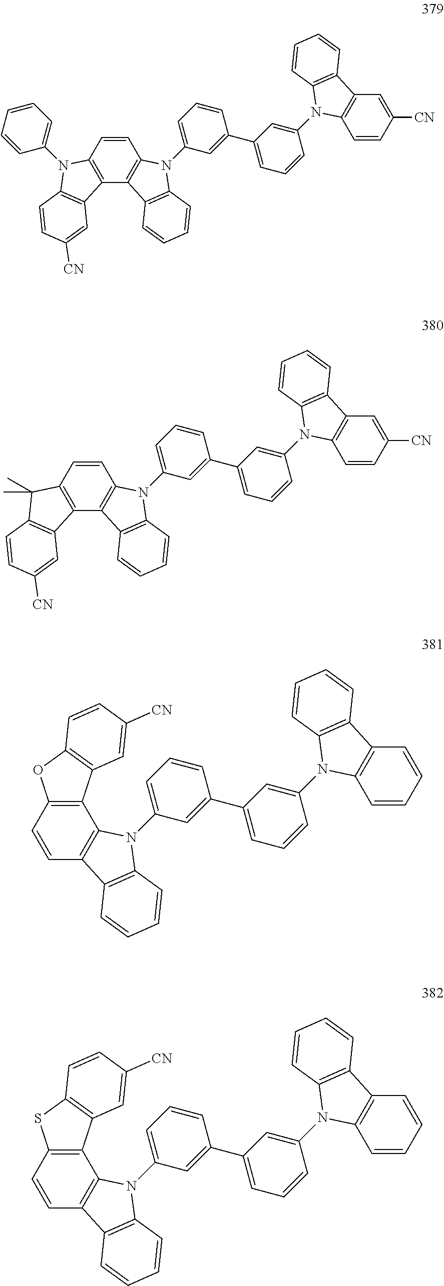 [SMILES: N#Cc1ccc2c(c1)c1ccccc1n2-c1cccc(-c2cccc(-n3c4ccccc4c4c5c6cc(C#N)ccc6n(-c6ccccc6)c5ccc43)c2)c1 ; CC1(C)c2ccc(C#N)cc2-c2c1ccc1c2c2ccccc2n1-c1cccc(-c2cccc(-n3c4ccccc4c4cc(C#N)ccc43)c2)c1 ; N#Cc1ccc2oc3ccc4c5ccccc5n(-c5cccc(-c6cccc(-n7c8ccccc8c8ccccc87)c6)c5)c4c3c2c1 ; N#Cc1ccc2sc3ccc4c5ccccc5n(-c5cccc(-c6cccc(-n7c8ccccc8c8ccccc87)c6)c5)c4c3c2c1]

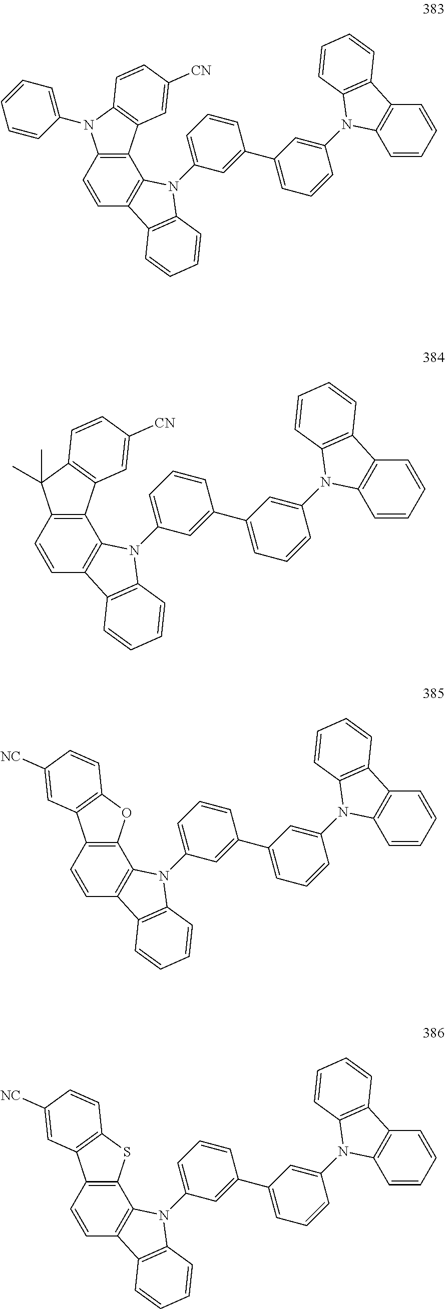 [SMILES: N#Cc1ccc2c(c1)c1c(ccc3c4ccccc4n(-c4cccc(-c5cccc(-n6c7ccccc7c7ccccc76)c5)c4)c31)n2-c1ccccc1 ; CC1(C)c2ccc(C#N)cc2-c2c1ccc1c3ccccc3n(-c3cccc(-c4cccc(-n5c6ccccc6c6ccccc65)c4)c3)c21 ; N#Cc1ccc2oc3c(ccc4c5ccccc5n(-c5cccc(-c6cccc(-n7c8ccccc8c8ccccc87)c6)c5)c43)c2c1 ; N#Cc1ccc2sc3c(ccc4c5ccccc5n(-c5cccc(-c6cccc(-n7c8ccccc8c8ccccc87)c6)c5)c43)c2c1]

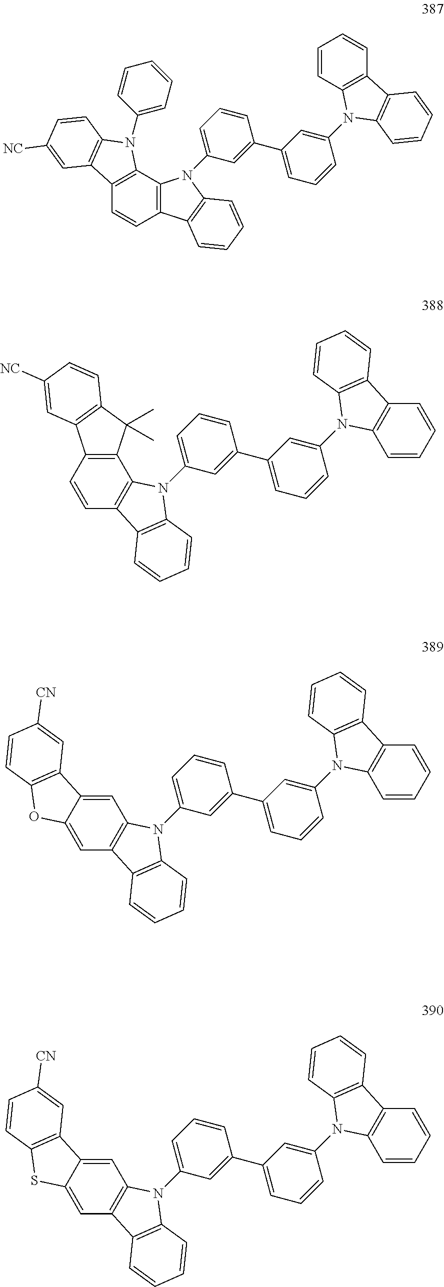 [SMILES: N#Cc1ccc2c(c1)c1ccc3c4ccccc4n(-c4cccc(-c5cccc(-n6c7ccccc7c7ccccc76)c5)c4)c3c1n2-c1ccccc1 ; CC1(C)c2ccc(C#N)cc2-c2ccc3c4ccccc4n(-c4cccc(-c5cccc(-n6c7ccccc7c7ccccc76)c5)c4)c3c21 ; N#Cc1ccc2oc3cc4c5ccccc5n(-c5cccc(-c6cccc(-n7c8ccccc8c8ccccc87)c6)c5)c4cc3c2c1 ; N#Cc1ccc2sc3cc4c5ccccc5n(-c5cccc(-c6cccc(-n7c8ccccc8c8ccccc87)c6)c5)c4cc3c2c1]

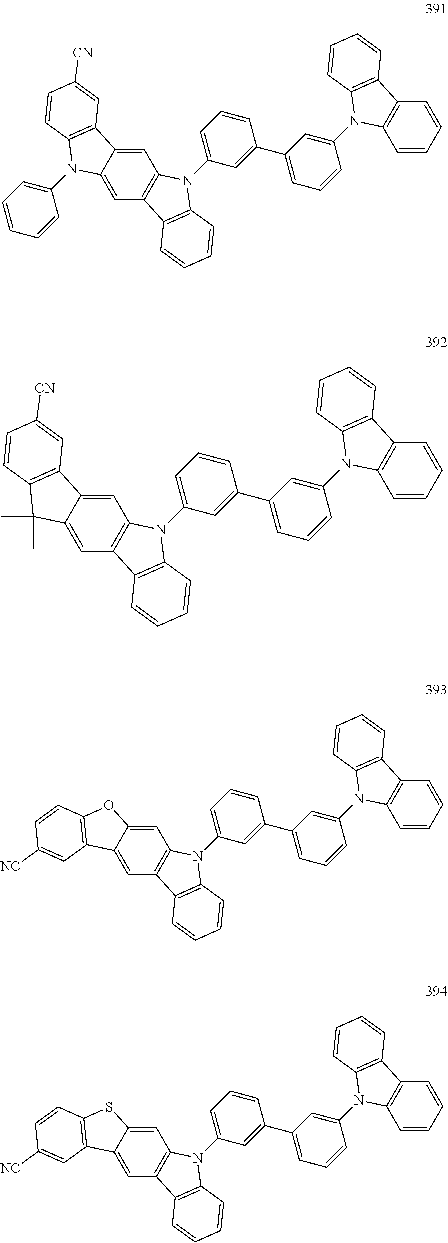 [SMILES: N#Cc1ccc2c(c1)c1cc3c(cc1n2-c1ccccc1)c1ccccc1n3-c1cccc(-c2cccc(-n3c4ccccc4c4ccccc43)c2)c1 ; CC1(C)c2ccc(C#N)cc2-c2cc3c(cc21)c1ccccc1n3-c1cccc(-c2cccc(-n3c4ccccc4c4ccccc43)c2)c1 ; N#Cc1ccc2oc3cc4c(cc3c2c1)c1ccccc1n4-c1cccc(-c2cccc(-n3c4ccccc4c4ccccc43)c2)c1 ; N#Cc1ccc2sc3cc4c(cc3c2c1)c1ccccc1n4-c1cccc(-c2cccc(-n3c4ccccc4c4ccccc43)c2)c1]

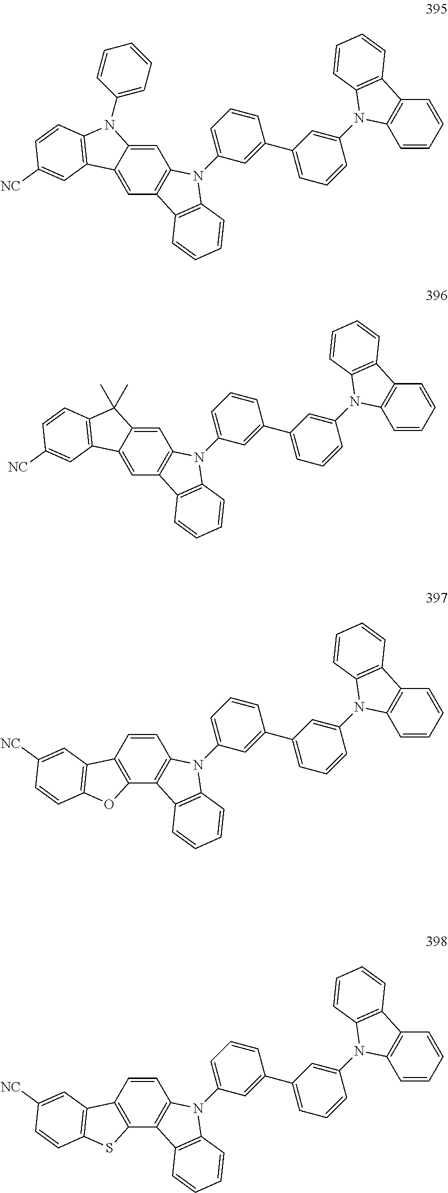 [SMILES: N#Cc1ccc2c(c1)c1cc3c4ccccc4n(-c4cccc(-c5cccc(-n6c7ccccc7c7ccccc76)c5)c4)c3cc1n2-c1ccccc1 ; CC1(C)c2ccc(C#N)cc2-c2cc3c4ccccc4n(-c4cccc(-c5cccc(-n6c7ccccc7c7ccccc76)c5)c4)c3cc21 ; N#Cc1ccc2oc3c(ccc4c3c3ccccc3n4-c3cccc(-c4cccc(-n5c6ccccc6c6ccccc65)c4)c3)c2c1 ; N#Cc1ccc2sc3c(ccc4c3c3ccccc3n4-c3cccc(-c4cccc(-n5c6ccccc6c6ccccc65)c4)c3)c2c1]

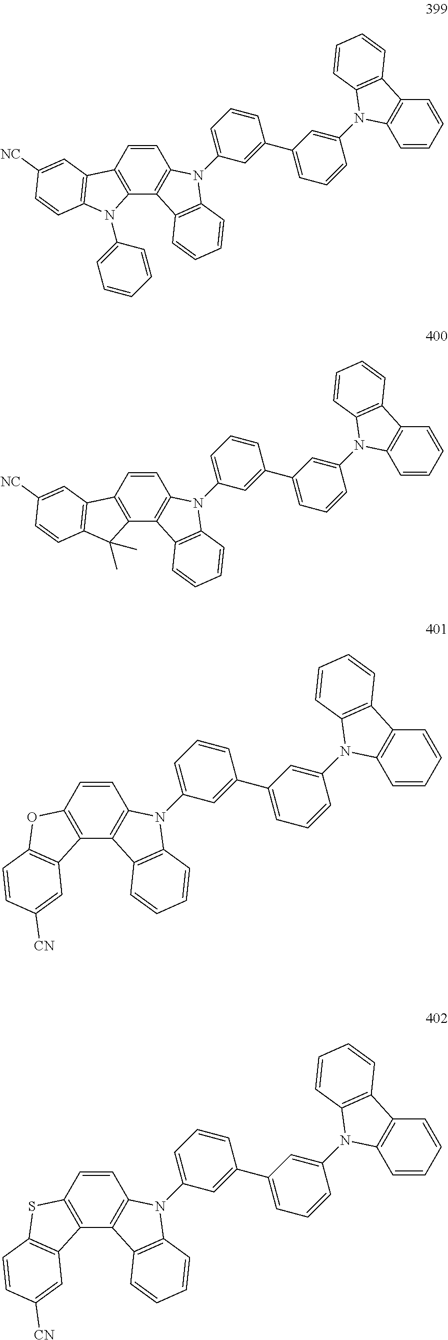 [SMILES: N#Cc1ccc2c(c1)c1ccc3c(c4ccccc4n3-c3cccc(-c4cccc(-n5c6ccccc6c6ccccc65)c4)c3)c1n2-c1ccccc1 ; CC1(C)c2ccc(C#N)cc2-c2ccc3c(c21)c1ccccc1n3-c1cccc(-c2cccc(-n3c4ccccc4c4ccccc43)c2)c1 ; N#Cc1ccc2oc3ccc4c(c5ccccc5n4-c4cccc(-c5cccc(-n6c7ccccc7c7ccccc76)c5)c4)c3c2c1 ; N#Cc1ccc2sc3ccc4c(c5ccccc5n4-c4cccc(-c5cccc(-n6c7ccccc7c7ccccc76)c5)c4)c3c2c1]

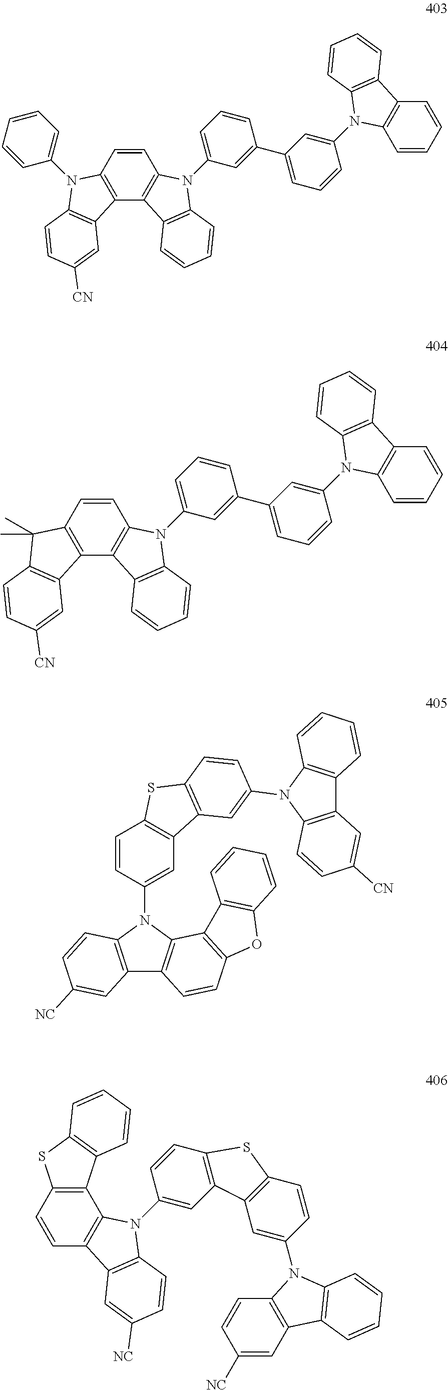 [SMILES: N#Cc1ccc2c(c1)c1c3c4ccccc4n(-c4cccc(-c5cccc(-n6c7ccccc7c7ccccc76)c5)c4)c3ccc1n2-c1ccccc1 ; CC1(C)c2ccc(C#N)cc2-c2c1ccc1c2c2ccccc2n1-c1cccc(-c2cccc(-n3c4ccccc4c4ccccc43)c2)c1 ; N#Cc1ccc2c(c1)c1ccccc1n2-c1ccc2sc3ccc(-n4c5ccc(C#N)cc5c5ccc6oc7ccccc7c6c54)cc3c2c1 ; N#Cc1ccc2c(c1)c1ccccc1n2-c1ccc2sc3ccc(-n4c5ccc(C#N)cc5c5ccc6sc7ccccc7c6c54)cc3c2c1]

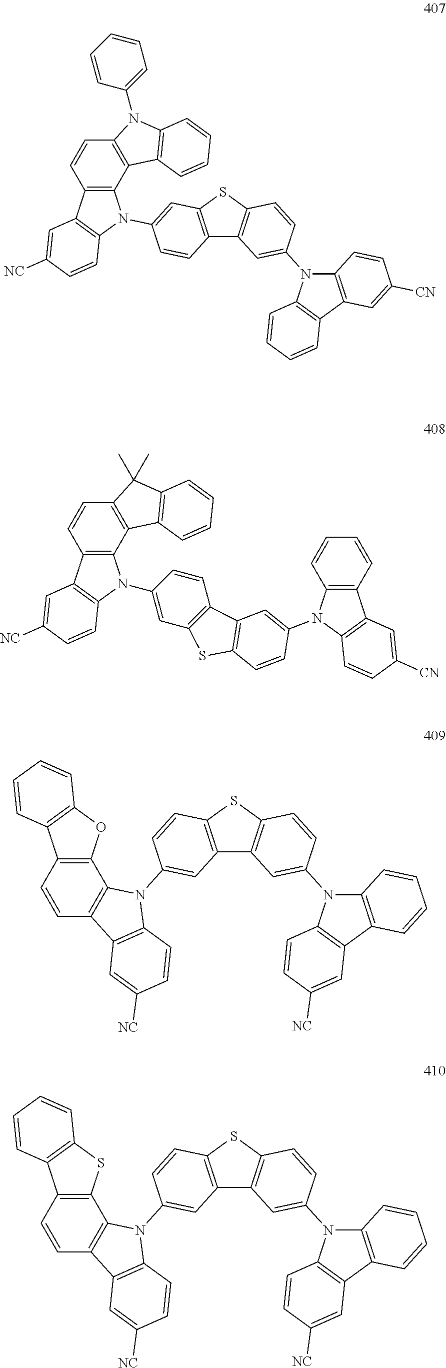 [SMILES: N#Cc1ccc2c(c1)c1ccccc1n2-c1ccc2sc3cc(-n4c5ccc(C#N)cc5c5ccc6c(c7ccccc7n6-c6ccccc6)c54)ccc3c2c1 ; CC1(C)c2ccccc2-c2c1ccc1c3cc(C#N)ccc3n(-c3ccc4c(c3)sc3ccc(-n5c6ccccc6c6cc(C#N)ccc65)cc34)c21 ; N#Cc1ccc2c(c1)c1ccccc1n2-c1ccc2sc3ccc(-n4c5ccc(C#N)cc5c5ccc6c7ccccc7oc6c54)cc3c2c1 ; N#Cc1ccc2c(c1)c1ccccc1n2-c1ccc2sc3ccc(-n4c5ccc(C#N)cc5c5ccc6c7ccccc7sc6c54)cc3c2c1]

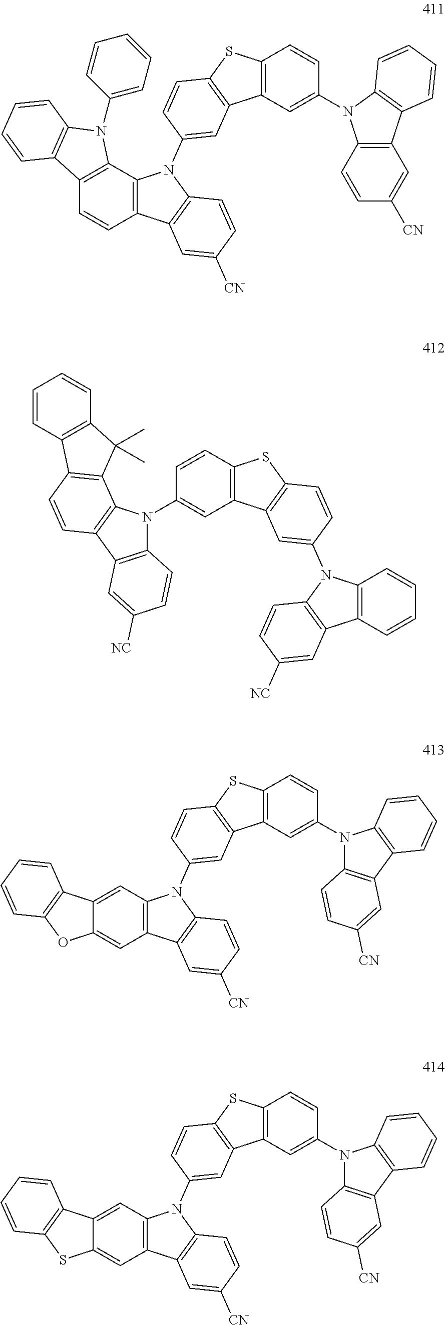 [SMILES: N#Cc1ccc2c(c1)c1ccccc1n2-c1ccc2sc3ccc(-n4c5ccc(C#N)cc5c5ccc6c7ccccc7n(-c7ccccc7)c6c54)cc3c2c1 ; CC1(C)c2ccccc2-c2ccc3c4cc(C#N)ccc4n(-c4ccc5sc6ccc(-n7c8ccccc8c8cc(C#N)ccc87)cc6c5c4)c3c21 ; N#Cc1ccc2c(c1)c1ccccc1n2-c1ccc2sc3ccc(-n4c5ccc(C#N)cc5c5cc6oc7ccccc7c6cc54)cc3c2c1 ; N#Cc1ccc2c(c1)c1ccccc1n2-c1ccc2sc3ccc(-n4c5ccc(C#N)cc5c5cc6sc7ccccc7c6cc54)cc3c2c1]

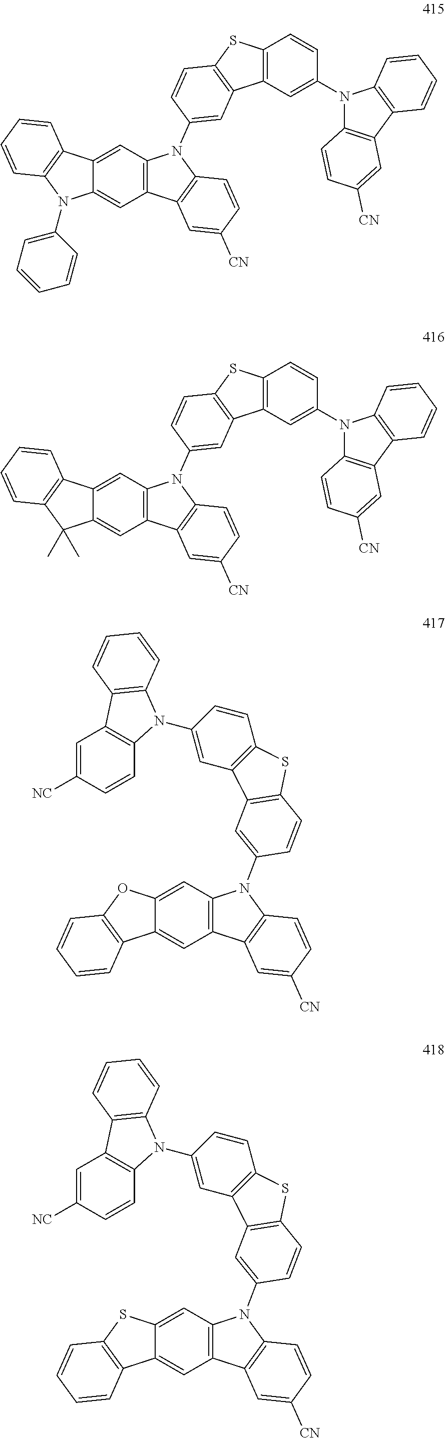 [SMILES: N#Cc1ccc2c(c1)c1ccccc1n2-c1ccc2sc3ccc(-n4c5ccc(C#N)cc5c5cc6c(cc54)c4ccccc4n6-c4ccccc4)cc3c2c1 ; CC1(C)c2ccccc2-c2cc3c(cc21)c1cc(C#N)ccc1n3-c1ccc2sc3ccc(-n4c5ccccc5c5cc(C#N)ccc54)cc3c2c1 ; N#Cc1ccc2c(c1)c1ccccc1n2-c1ccc2sc3ccc(-n4c5ccc(C#N)cc5c5cc6c(cc54)oc4ccccc46)cc3c2c1 ; N#Cc1ccc2c(c1)c1ccccc1n2-c1ccc2sc3ccc(-n4c5ccc(C#N)cc5c5cc6c(cc54)sc4ccccc46)cc3c2c1]

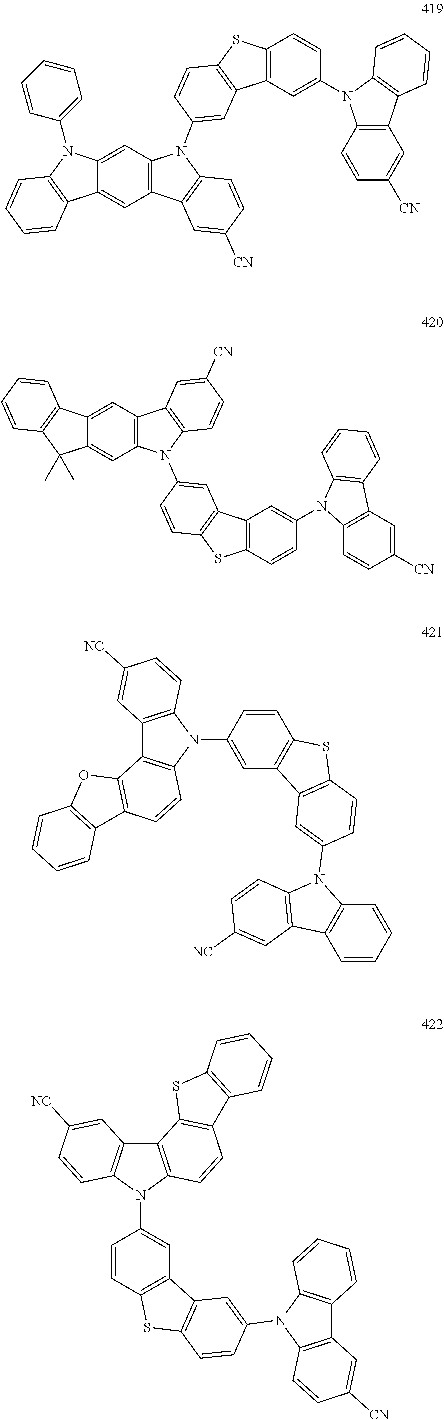 [SMILES: N#Cc1ccc2c(c1)c1ccccc1n2-c1ccc2sc3ccc(-n4c5ccc(C#N)cc5c5cc6c7ccccc7n(-c7ccccc7)c6cc54)cc3c2c1 ; CC1(C)c2ccccc2-c2cc3c4cc(C#N)ccc4n(-c4ccc5sc6ccc(-n7c8ccccc8c8cc(C#N)ccc87)cc6c5c4)c3cc21 ; N#Cc1ccc2c(c1)c1ccccc1n2-c1ccc2sc3ccc(-n4c5ccc(C#N)cc5c5c6oc7ccccc7c6ccc54)cc3c2c1 ; N#Cc1ccc2c(c1)c1ccccc1n2-c1ccc2sc3ccc(-n4c5ccc(C#N)cc5c5c6sc7ccccc7c6ccc54)cc3c2c1]

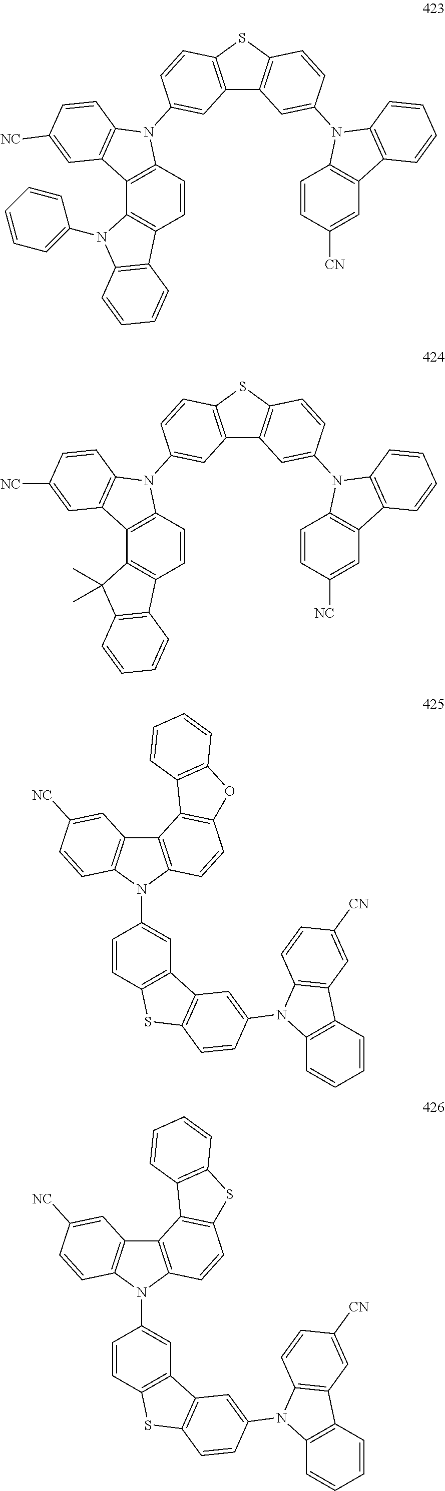 [SMILES: N#Cc1ccc2c(c1)c1ccccc1n2-c1ccc2sc3ccc(-n4c5ccc(C#N)cc5c5c4ccc4c6ccccc6n(-c6ccccc6)c45)cc3c2c1 ; CC1(C)c2ccccc2-c2ccc3c(c21)c1cc(C#N)ccc1n3-c1ccc2sc3ccc(-n4c5ccccc5c5cc(C#N)ccc54)cc3c2c1 ; N#Cc1ccc2c(c1)c1ccccc1n2-c1ccc2sc3ccc(-n4c5ccc(C#N)cc5c5c6c(ccc54)oc4ccccc46)cc3c2c1 ; N#Cc1ccc2c(c1)c1ccccc1n2-c1ccc2sc3ccc(-n4c5ccc(C#N)cc5c5c6c(ccc54)sc4ccccc46)cc3c2c1]

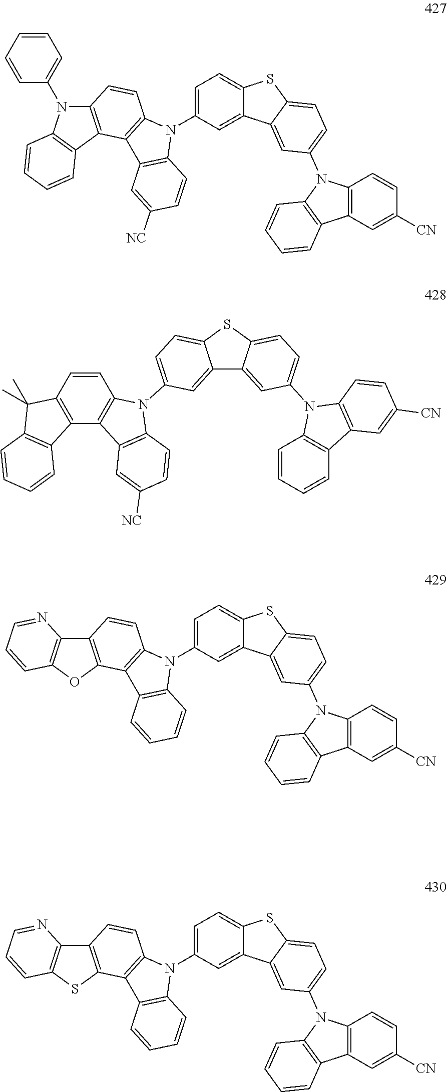 [SMILES: N#Cc1ccc2c(c1)c1ccccc1n2-c1ccc2sc3ccc(-n4c5ccc(C#N)cc5c5c6c7ccccc7n(-c7ccccc7)c6ccc54)cc3c2c1 ; CC1(C)c2ccccc2-c2c1ccc1c2c2cc(C#N)ccc2n1-c1ccc2sc3ccc(-n4c5ccccc5c5cc(C#N)ccc54)cc3c2c1 ; N#Cc1ccc2c(c1)c1ccccc1n2-c1ccc2sc3ccc(-n4c5ccccc5c5c6oc7cccnc7c6ccc54)cc3c2c1 ; N#Cc1ccc2c(c1)c1ccccc1n2-c1ccc2sc3ccc(-n4c5ccccc5c5c6sc7cccnc7c6ccc54)cc3c2c1]

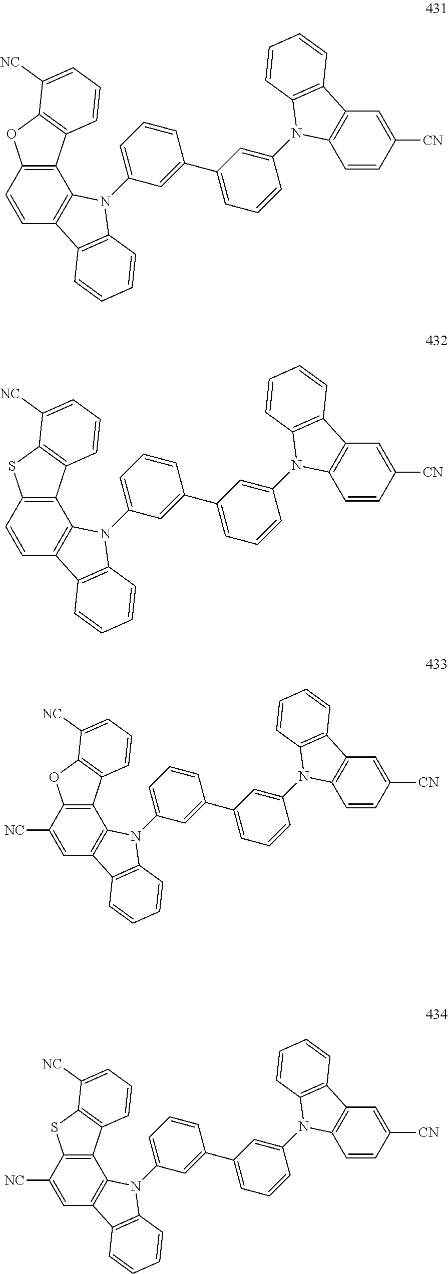 [SMILES: N#Cc1ccc2c(c1)c1ccccc1n2-c1cccc(-c2cccc(-n3c4ccccc4c4ccc5oc6c(C#N)cccc6c5c43)c2)c1 ; N#Cc1ccc2c(c1)c1ccccc1n2-c1cccc(-c2cccc(-n3c4ccccc4c4ccc5sc6c(C#N)cccc6c5c43)c2)c1 ; N#Cc1ccc2c(c1)c1ccccc1n2-c1cccc(-c2cccc(-n3c4ccccc4c4cc(C#N)c5oc6c(C#N)cccc6c5c43)c2)c1 ; N#Cc1ccc2c(c1)c1ccccc1n2-c1cccc(-c2cccc(-n3c4ccccc4c4cc(C#N)c5sc6c(C#N)cccc6c5c43)c2)c1]

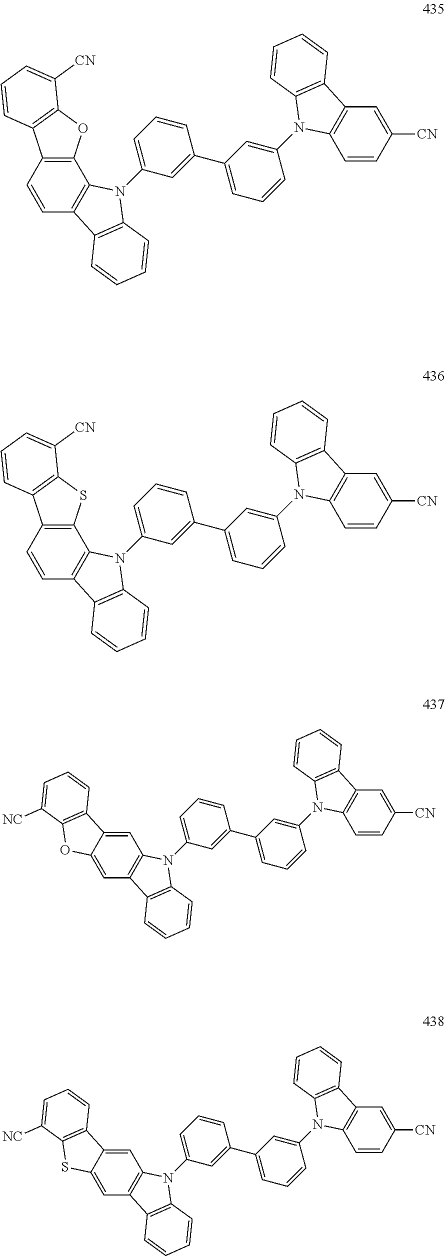 [SMILES: N#Cc1ccc2c(c1)c1ccccc1n2-c1cccc(-c2cccc(-n3c4ccccc4c4ccc5c6cccc(C#N)c6oc5c43)c2)c1 ; N#Cc1ccc2c(c1)c1ccccc1n2-c1cccc(-c2cccc(-n3c4ccccc4c4ccc5c6cccc(C#N)c6sc5c43)c2)c1 ; N#Cc1ccc2c(c1)c1ccccc1n2-c1cccc(-c2cccc(-n3c4ccccc4c4cc5oc6c(C#N)cccc6c5cc43)c2)c1 ; N#Cc1ccc2c(c1)c1ccccc1n2-c1cccc(-c2cccc(-n3c4ccccc4c4cc5sc6c(C#N)cccc6c5cc43)c2)c1]

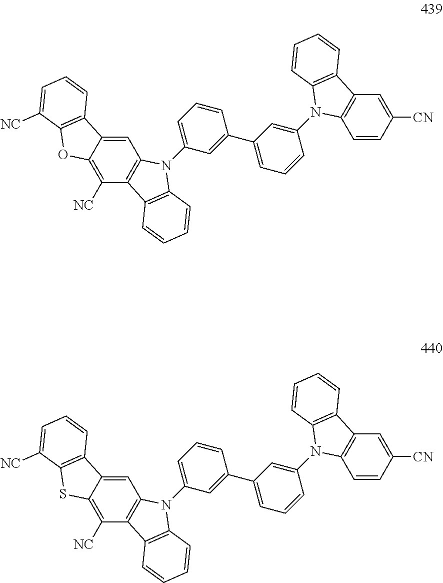 [SMILES: N#Cc1ccc2c(c1)c1ccccc1n2-c1cccc(-c2cccc(-n3c4ccccc4c4c(C#N)c5oc6c(C#N)cccc6c5cc43)c2)c1 ; N#Cc1ccc2c(c1)c1ccccc1n2-c1cccc(-c2cccc(-n3c4ccccc4c4c(C#N)c5sc6c(C#N)cccc6c5cc43)c2)c1]

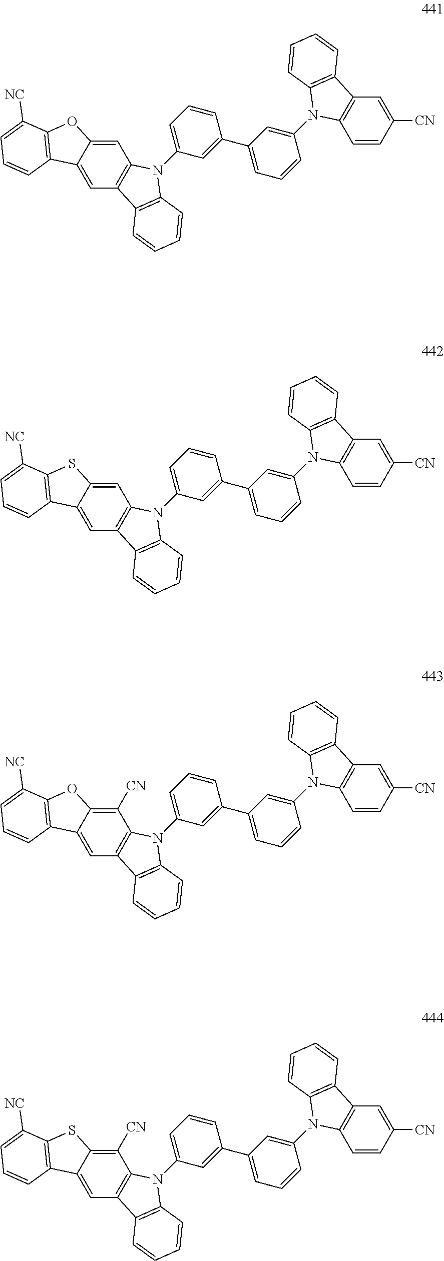 [SMILES: N#Cc1ccc2c(c1)c1ccccc1n2-c1cccc(-c2cccc(-n3c4ccccc4c4cc5c(cc43)oc3c(C#N)cccc35)c2)c1 ; N#Cc1ccc2c(c1)c1ccccc1n2-c1cccc(-c2cccc(-n3c4ccccc4c4cc5c(cc43)sc3c(C#N)cccc35)c2)c1 ; N#Cc1ccc2c(c1)c1ccccc1n2-c1cccc(-c2cccc(-n3c4ccccc4c4cc5c(oc6c(C#N)cccc65)c(C#N)c43)c2)c1 ; N#Cc1ccc2c(c1)c1ccccc1n2-c1cccc(-c2cccc(-n3c4ccccc4c4cc5c(sc6c(C#N)cccc65)c(C#N)c43)c2)c1]

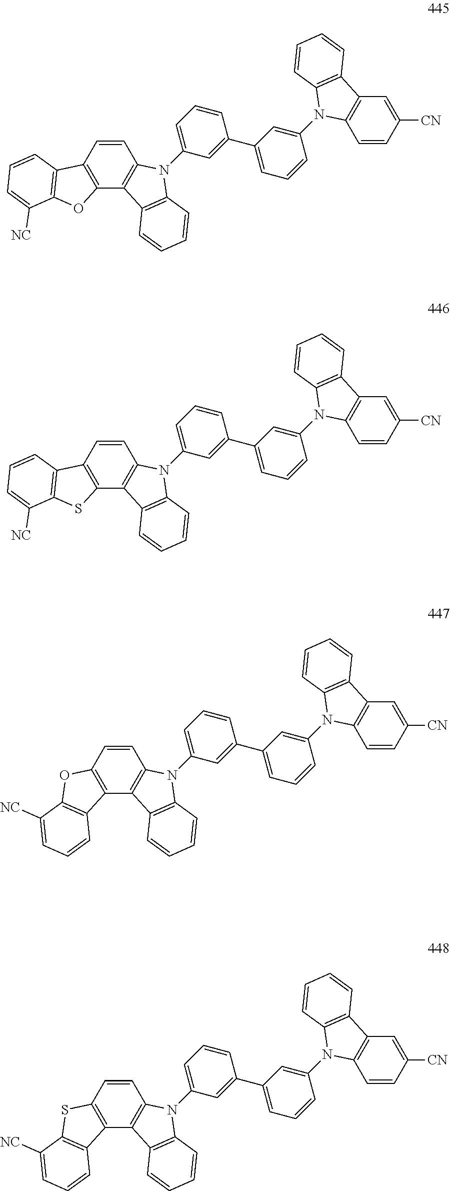 [SMILES: N#Cc1ccc2c(c1)c1ccccc1n2-c1cccc(-c2cccc(-n3c4ccccc4c4c5oc6c(C#N)cccc6c5ccc43)c2)c1 ; N#Cc1ccc2c(c1)c1ccccc1n2-c1cccc(-c2cccc(-n3c4ccccc4c4c5sc6c(C#N)cccc6c5ccc43)c2)c1 ; N#Cc1ccc2c(c1)c1ccccc1n2-c1cccc(-c2cccc(-n3c4ccccc4c4c5c(ccc43)oc3c(C#N)cccc35)c2)c1 ; N#Cc1ccc2c(c1)c1ccccc1n2-c1cccc(-c2cccc(-n3c4ccccc4c4c5c(ccc43)sc3c(C#N)cccc35)c2)c1]

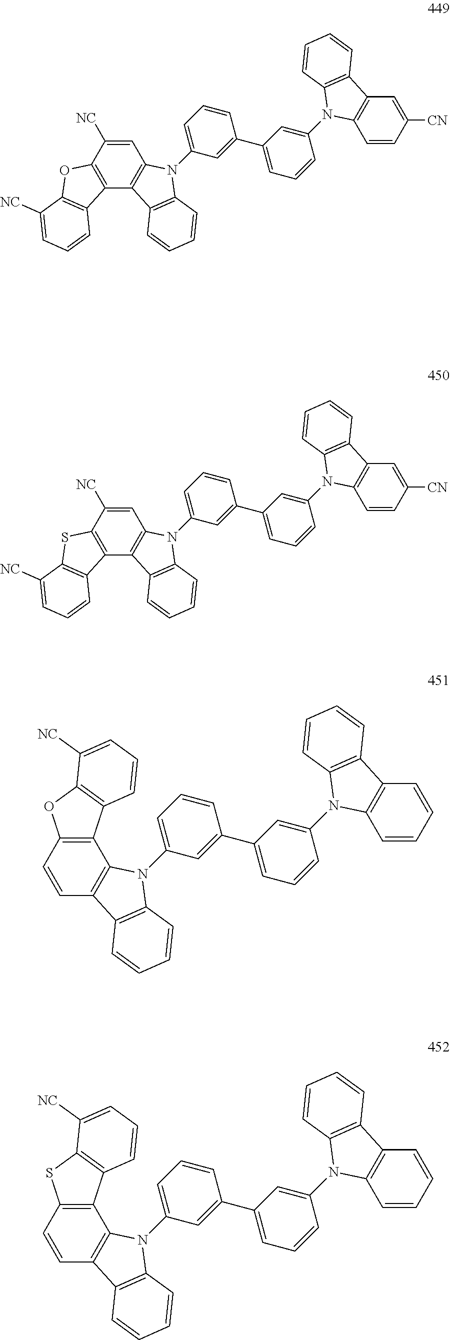 [SMILES: N#Cc1ccc2c(c1)c1ccccc1n2-c1cccc(-c2cccc(-n3c4ccccc4c4c5c(oc6c(C#N)cccc65)c(C#N)cc43)c2)c1 ; N#Cc1ccc2c(c1)c1ccccc1n2-c1cccc(-c2cccc(-n3c4ccccc4c4c5c(sc6c(C#N)cccc65)c(C#N)cc43)c2)c1 ; N#Cc1cccc2c1oc1ccc3c4ccccc4n(-c4cccc(-c5cccc(-n6c7ccccc7c7ccccc76)c5)c4)c3c12 ; N#Cc1cccc2c1sc1ccc3c4ccccc4n(-c4cccc(-c5cccc(-n6c7ccccc7c7ccccc76)c5)c4)c3c12]

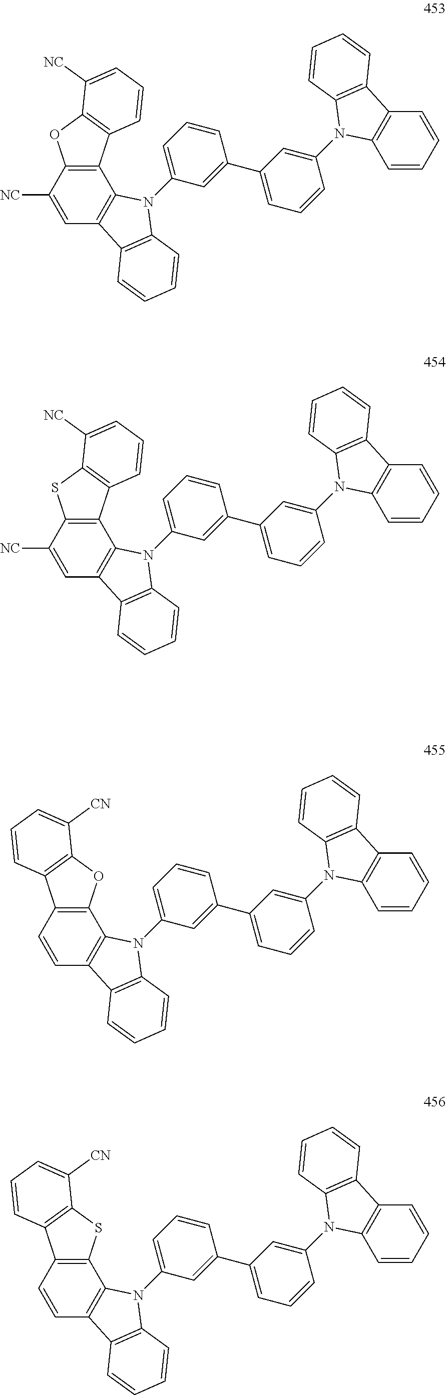 [SMILES: N#Cc1cccc2c1oc1c(C#N)cc3c4ccccc4n(-c4cccc(-c5cccc(-n6c7ccccc7c7ccccc76)c5)c4)c3c12 ; N#Cc1cccc2c1sc1c(C#N)cc3c4ccccc4n(-c4cccc(-c5cccc(-n6c7ccccc7c7ccccc76)c5)c4)c3c12 ; N#Cc1cccc2c1oc1c2ccc2c3ccccc3n(-c3cccc(-c4cccc(-n5c6ccccc6c6ccccc65)c4)c3)c21 ; N#Cc1cccc2c1sc1c2ccc2c3ccccc3n(-c3cccc(-c4cccc(-n5c6ccccc6c6ccccc65)c4)c3)c21]

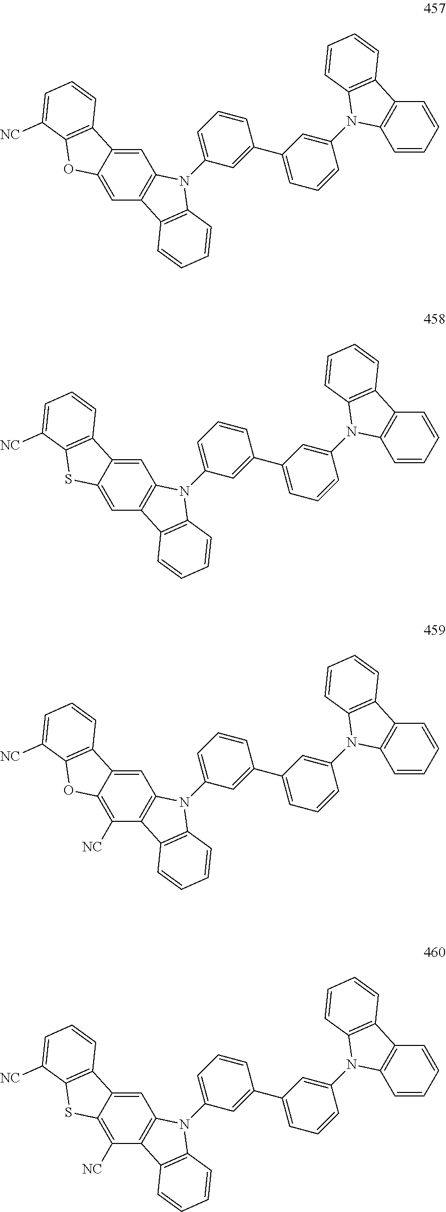 [SMILES: N#Cc1cccc2c1oc1cc3c4ccccc4n(-c4cccc(-c5cccc(-n6c7ccccc7c7ccccc76)c5)c4)c3cc12 ; N#Cc1cccc2c1sc1cc3c4ccccc4n(-c4cccc(-c5cccc(-n6c7ccccc7c7ccccc76)c5)c4)c3cc12 ; N#Cc1cccc2c1oc1c(C#N)c3c4ccccc4n(-c4cccc(-c5cccc(-n6c7ccccc7c7ccccc76)c5)c4)c3cc12 ; N#Cc1cccc2c1sc1c(C#N)c3c4ccccc4n(-c4cccc(-c5cccc(-n6c7ccccc7c7ccccc76)c5)c4)c3cc12]

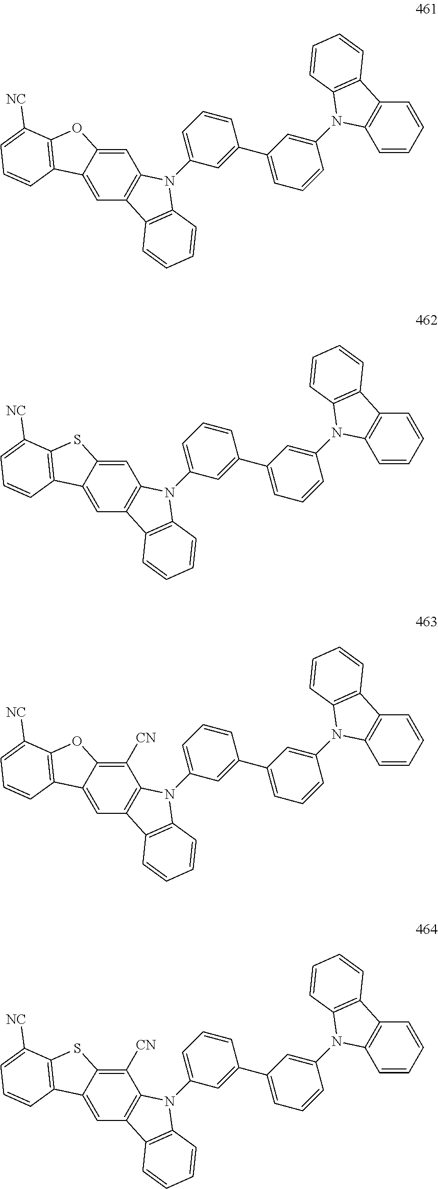 [SMILES: N#Cc1cccc2c1oc1cc3c(cc12)c1ccccc1n3-c1cccc(-c2cccc(-n3c4ccccc4c4ccccc43)c2)c1 ; N#Cc1cccc2c1sc1cc3c(cc12)c1ccccc1n3-c1cccc(-c2cccc(-n3c4ccccc4c4ccccc43)c2)c1 ; N#Cc1cccc2c1oc1c(C#N)c3c(cc12)c1ccccc1n3-c1cccc(-c2cccc(-n3c4ccccc4c4ccccc43)c2)c1 ; N#Cc1cccc2c1sc1c(C#N)c3c(cc12)c1ccccc1n3-c1cccc(-c2cccc(-n3c4ccccc4c4ccccc43)c2)c1]

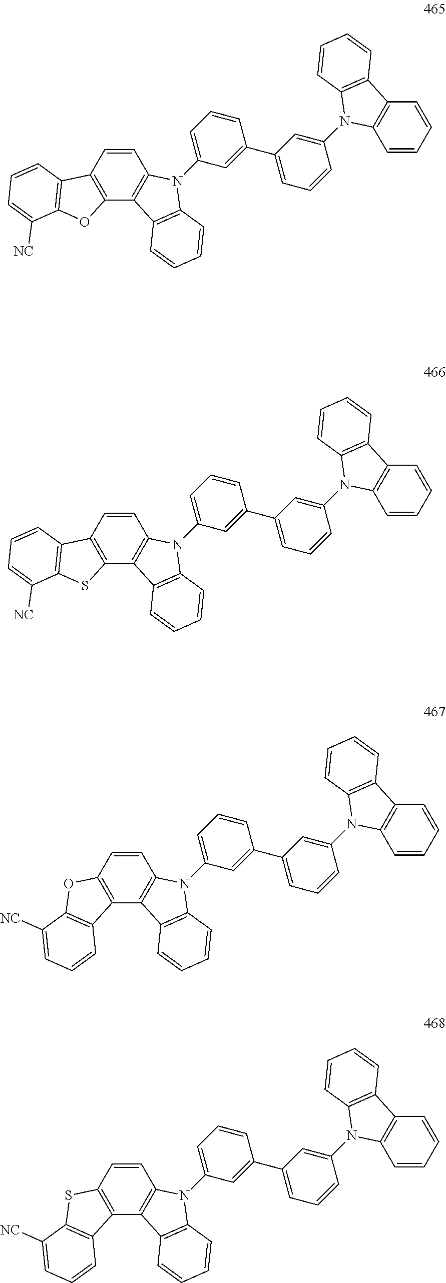 [SMILES: N#Cc1cccc2c1oc1c2ccc2c1c1ccccc1n2-c1cccc(-c2cccc(-n3c4ccccc4c4ccccc43)c2)c1 ; N#Cc1cccc2c1sc1c2ccc2c1c1ccccc1n2-c1cccc(-c2cccc(-n3c4ccccc4c4ccccc43)c2)c1 ; N#Cc1cccc2c1oc1ccc3c(c4ccccc4n3-c3cccc(-c4cccc(-n5c6ccccc6c6ccccc65)c4)c3)c12 ; N#Cc1cccc2c1sc1ccc3c(c4ccccc4n3-c3cccc(-c4cccc(-n5c6ccccc6c6ccccc65)c4)c3)c12]

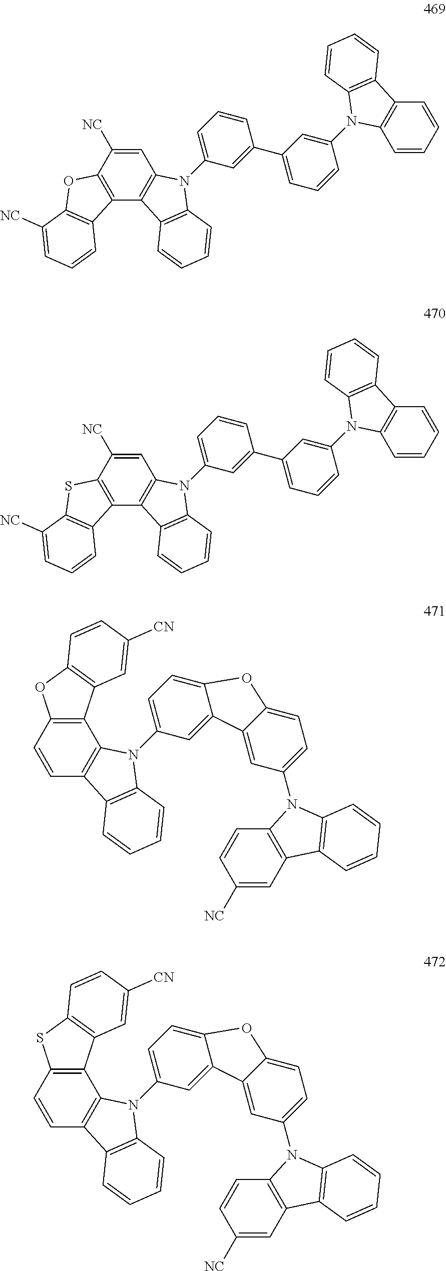 [SMILES: N#Cc1cccc2c1oc1c(C#N)cc3c(c4ccccc4n3-c3cccc(-c4cccc(-n5c6ccccc6c6ccccc65)c4)c3)c12 ; N#Cc1cccc2c1sc1c(C#N)cc3c(c4ccccc4n3-c3cccc(-c4cccc(-n5c6ccccc6c6ccccc65)c4)c3)c12 ; N#Cc1ccc2oc3ccc4c5ccccc5n(-c5ccc6oc7ccc(-n8c9ccccc9c9cc(C#N)ccc98)cc7c6c5)c4c3c2c1 ; N#Cc1ccc2sc3ccc4c5ccccc5n(-c5ccc6oc7ccc(-n8c9ccccc9c9cc(C#N)ccc98)cc7c6c5)c4c3c2c1]

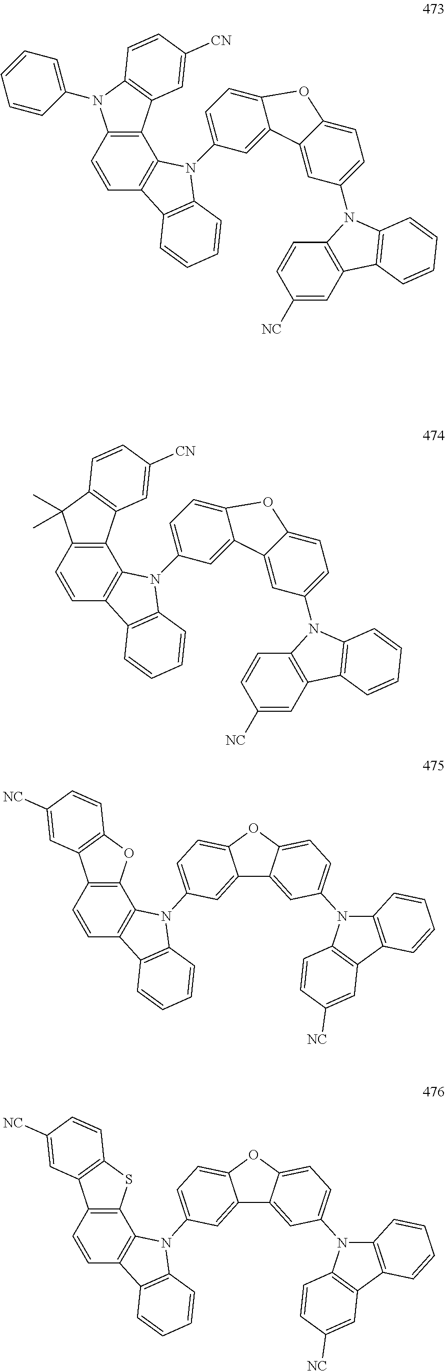 [SMILES: N#Cc1ccc2c(c1)c1ccccc1n2-c1ccc2oc3ccc(-n4c5ccccc5c5ccc6c(c7cc(C#N)ccc7n6-c6ccccc6)c54)cc3c2c1 ; CC1(C)c2ccc(C#N)cc2-c2c1ccc1c3ccccc3n(-c3ccc4oc5ccc(-n6c7ccccc7c7cc(C#N)ccc76)cc5c4c3)c21 ; N#Cc1ccc2oc3c(ccc4c5ccccc5n(-c5ccc6oc7ccc(-n8c9ccccc9c9cc(C#N)ccc98)cc7c6c5)c43)c2c1 ; N#Cc1ccc2sc3c(ccc4c5ccccc5n(-c5ccc6oc7ccc(-n8c9ccccc9c9cc(C#N)ccc98)cc7c6c5)c43)c2c1]

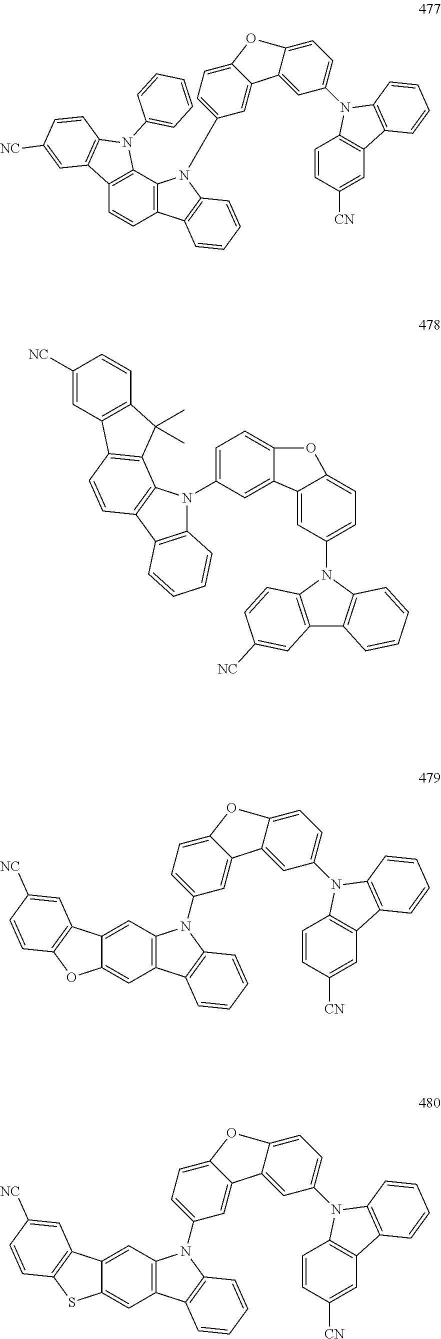 [SMILES: N#Cc1ccc2c(c1)c1ccccc1n2-c1ccc2oc3ccc(-n4c5ccccc5c5ccc6c7cc(C#N)ccc7n(-c7ccccc7)c6c54)cc3c2c1 ; CC1(C)c2ccc(C#N)cc2-c2ccc3c4ccccc4n(-c4ccc5oc6ccc(-n7c8ccccc8c8cc(C#N)ccc87)cc6c5c4)c3c21 ; N#Cc1ccc2oc3cc4c5ccccc5n(-c5ccc6oc7ccc(-n8c9ccccc9c9cc(C#N)ccc98)cc7c6c5)c4cc3c2c1 ; N#Cc1ccc2sc3cc4c5ccccc5n(-c5ccc6oc7ccc(-n8c9ccccc9c9cc(C#N)ccc98)cc7c6c5)c4cc3c2c1]

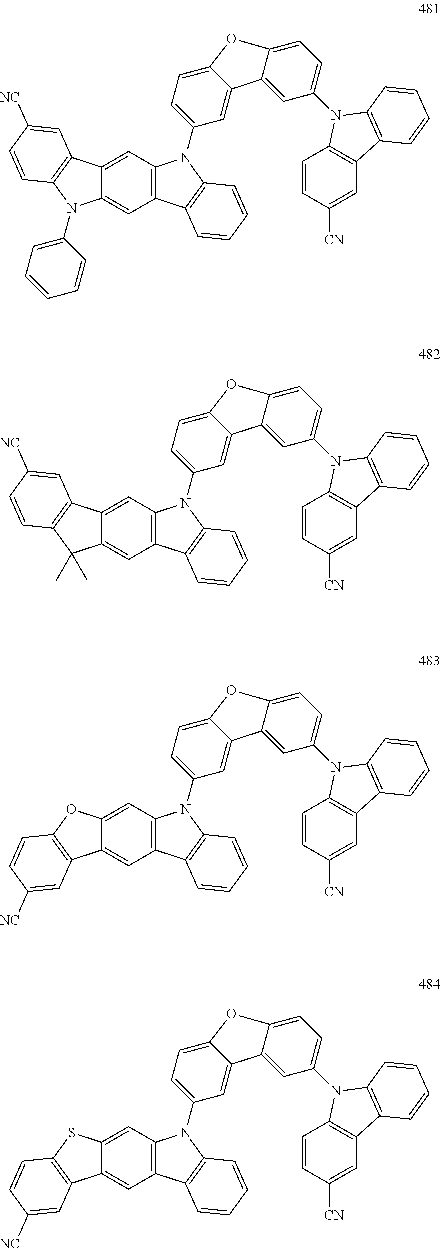 [SMILES: N#Cc1ccc2c(c1)c1ccccc1n2-c1ccc2oc3ccc(-n4c5ccccc5c5cc6c(cc54)c4cc(C#N)ccc4n6-c4ccccc4)cc3c2c1 ; CC1(C)c2ccc(C#N)cc2-c2cc3c(cc21)c1ccccc1n3-c1ccc2oc3ccc(-n4c5ccccc5c5cc(C#N)ccc54)cc3c2c1 ; N#Cc1ccc2oc3cc4c(cc3c2c1)c1ccccc1n4-c1ccc2oc3ccc(-n4c5ccccc5c5cc(C#N)ccc54)cc3c2c1 ; N#Cc1ccc2sc3cc4c(cc3c2c1)c1ccccc1n4-c1ccc2oc3ccc(-n4c5ccccc5c5cc(C#N)ccc54)cc3c2c1]

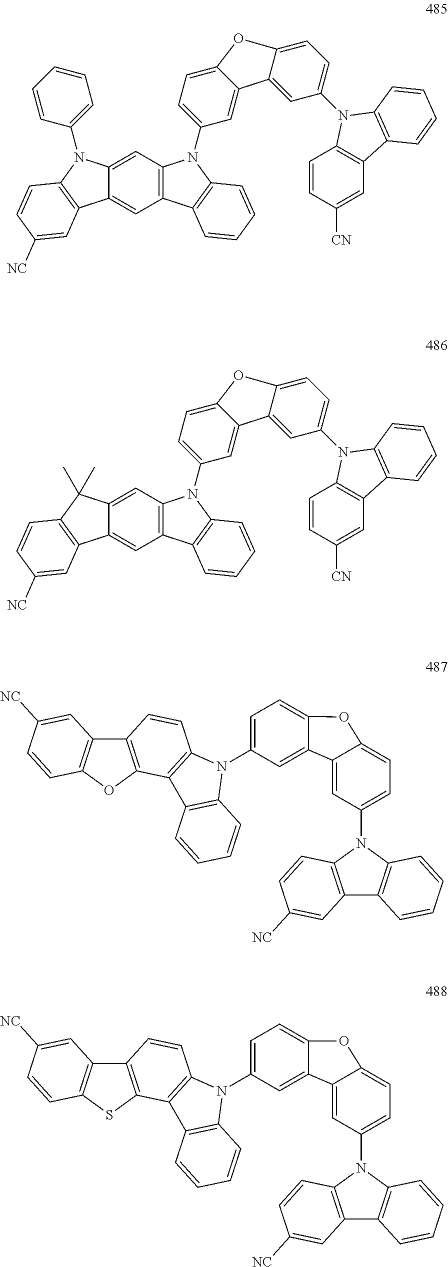 [SMILES: N#Cc1ccc2c(c1)c1ccccc1n2-c1ccc2oc3ccc(-n4c5ccccc5c5cc6c7cc(C#N)ccc7n(-c7ccccc7)c6cc54)cc3c2c1 ; CC1(C)c2ccc(C#N)cc2-c2cc3c4ccccc4n(-c4ccc5oc6ccc(-n7c8ccccc8c8cc(C#N)ccc87)cc6c5c4)c3cc21 ; N#Cc1ccc2oc3c(ccc4c3c3ccccc3n4-c3ccc4oc5ccc(-n6c7ccccc7c7cc(C#N)ccc76)cc5c4c3)c2c1 ; N#Cc1ccc2sc3c(ccc4c3c3ccccc3n4-c3ccc4oc5ccc(-n6c7ccccc7c7cc(C#N)ccc76)cc5c4c3)c2c1]

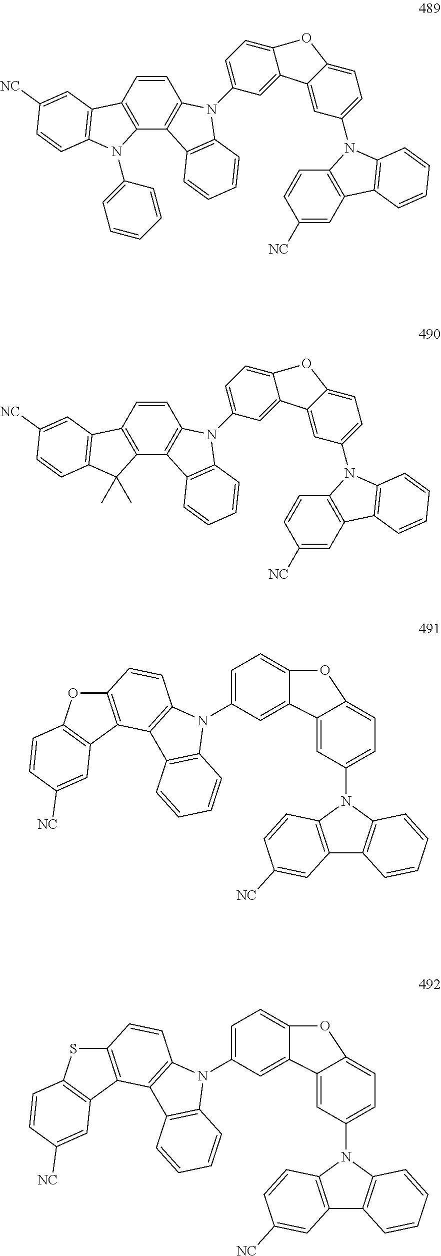 [SMILES: N#Cc1ccc2c(c1)c1ccccc1n2-c1ccc2oc3ccc(-n4c5ccccc5c5c4ccc4c6cc(C#N)ccc6n(-c6ccccc6)c45)cc3c2c1 ; CC1(C)c2ccc(C#N)cc2-c2ccc3c(c21)c1ccccc1n3-c1ccc2oc3ccc(-n4c5ccccc5c5cc(C#N)ccc54)cc3c2c1 ; N#Cc1ccc2oc3ccc4c(c5ccccc5n4-c4ccc5oc6ccc(-n7c8ccccc8c8cc(C#N)ccc87)cc6c5c4)c3c2c1 ; N#Cc1ccc2sc3ccc4c(c5ccccc5n4-c4ccc5oc6ccc(-n7c8ccccc8c8cc(C#N)ccc87)cc6c5c4)c3c2c1]

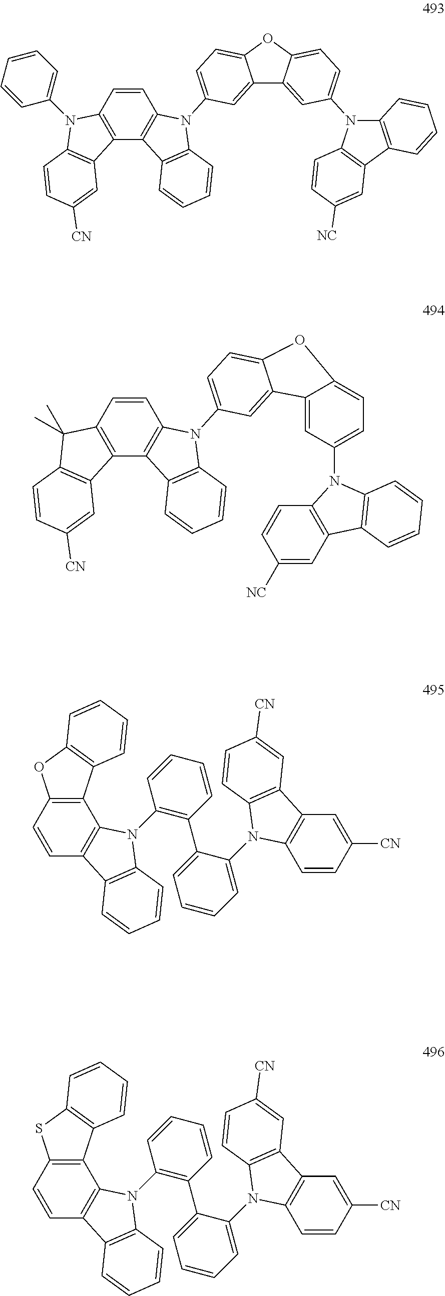 [SMILES: N#Cc1ccc2c(c1)c1ccccc1n2-c1ccc2oc3ccc(-n4c5ccccc5c5c6c7cc(C#N)ccc7n(-c7ccccc7)c6ccc54)cc3c2c1 ; CC1(C)c2ccc(C#N)cc2-c2c1ccc1c2c2ccccc2n1-c1ccc2oc3ccc(-n4c5ccccc5c5cc(C#N)ccc54)cc3c2c1 ; N#Cc1ccc2c(c1)c1cc(C#N)ccc1n2-c1ccccc1-c1ccccc1-n1c2ccccc2c2ccc3oc4ccccc4c3c21 ; N#Cc1ccc2c(c1)c1cc(C#N)ccc1n2-c1ccccc1-c1ccccc1-n1c2ccccc2c2ccc3sc4ccccc4c3c21]

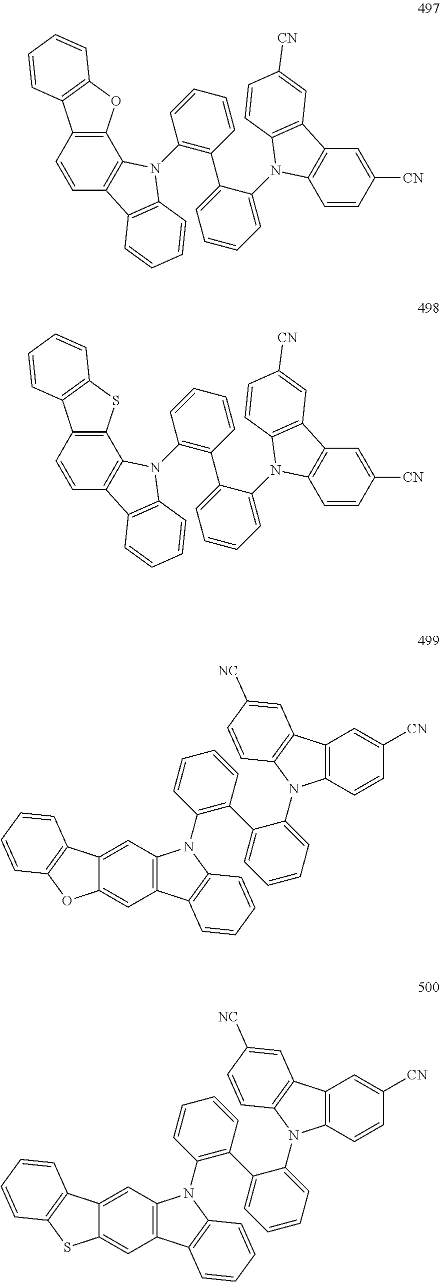 [SMILES: N#Cc1ccc2c(c1)c1cc(C#N)ccc1n2-c1ccccc1-c1ccccc1-n1c2ccccc2c2ccc3c4ccccc4oc3c21 ; N#Cc1ccc2c(c1)c1cc(C#N)ccc1n2-c1ccccc1-c1ccccc1-n1c2ccccc2c2ccc3c4ccccc4sc3c21 ; N#Cc1ccc2c(c1)c1cc(C#N)ccc1n2-c1ccccc1-c1ccccc1-n1c2ccccc2c2cc3oc4ccccc4c3cc21 ; N#Cc1ccc2c(c1)c1cc(C#N)ccc1n2-c1ccccc1-c1ccccc1-n1c2ccccc2c2cc3sc4ccccc4c3cc21]

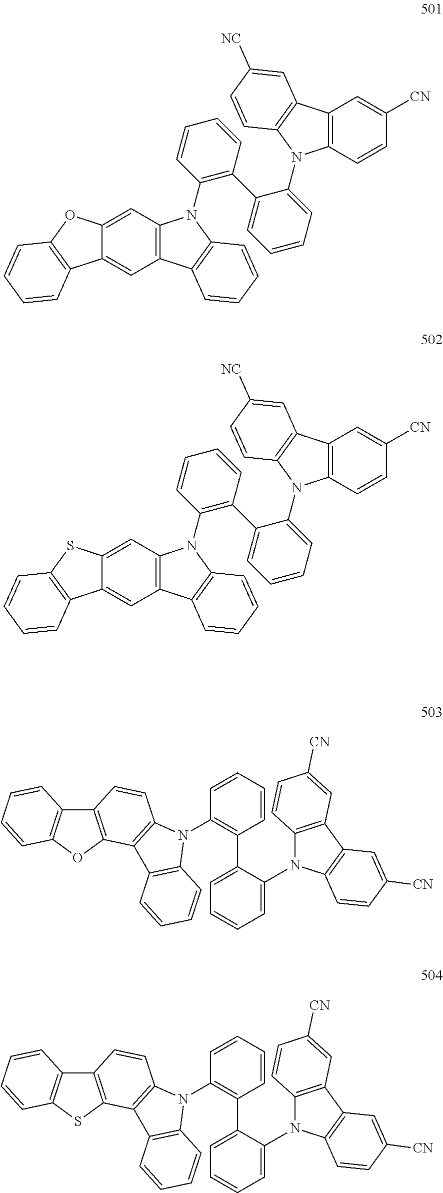 [SMILES: N#Cc1ccc2c(c1)c1cc(C#N)ccc1n2-c1ccccc1-c1ccccc1-n1c2ccccc2c2cc3c(cc21)oc1ccccc13 ; N#Cc1ccc2c(c1)c1cc(C#N)ccc1n2-c1ccccc1-c1ccccc1-n1c2ccccc2c2cc3c(cc21)sc1ccccc13 ; N#Cc1ccc2c(c1)c1cc(C#N)ccc1n2-c1ccccc1-c1ccccc1-n1c2ccccc2c2c3oc4ccccc4c3ccc21 ; N#Cc1ccc2c(c1)c1cc(C#N)ccc1n2-c1ccccc1-c1ccccc1-n1c2ccccc2c2c3sc4ccccc4c3ccc21]

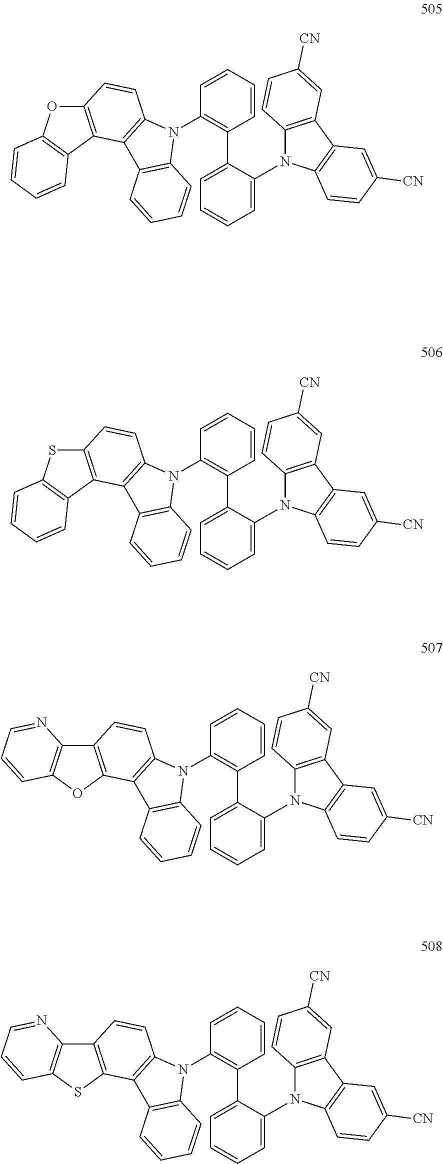 [SMILES: N#Cc1ccc2c(c1)c1cc(C#N)ccc1n2-c1ccccc1-c1ccccc1-n1c2ccccc2c2c3c(ccc21)oc1ccccc13 ; N#Cc1ccc2c(c1)c1cc(C#N)ccc1n2-c1ccccc1-c1ccccc1-n1c2ccccc2c2c3c(ccc21)sc1ccccc13 ; N#Cc1ccc2c(c1)c1cc(C#N)ccc1n2-c1ccccc1-c1ccccc1-n1c2ccccc2c2c3oc4cccnc4c3ccc21 ; N#Cc1ccc2c(c1)c1cc(C#N)ccc1n2-c1ccccc1-c1ccccc1-n1c2ccccc2c2c3sc4cccnc4c3ccc21]

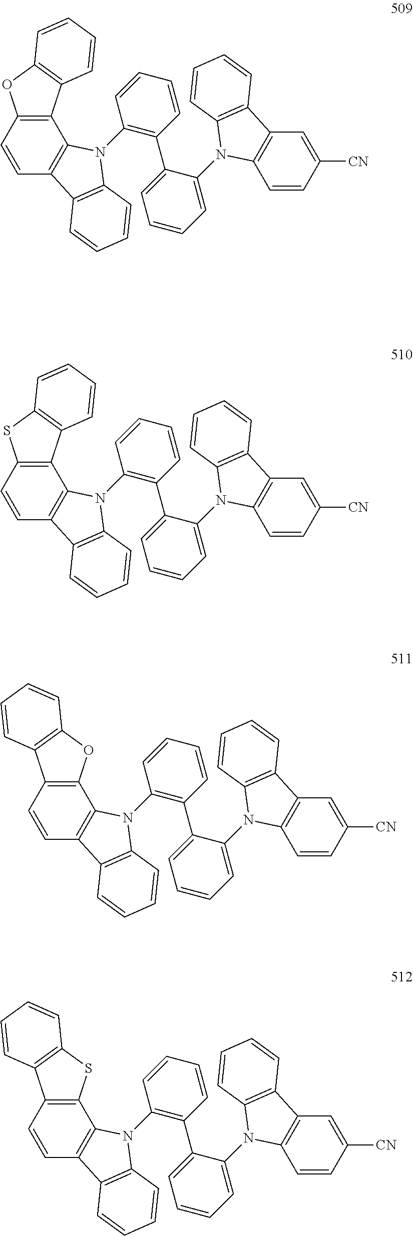 [SMILES: N#Cc1ccc2c(c1)c1ccccc1n2-c1ccccc1-c1ccccc1-n1c2ccccc2c2ccc3oc4ccccc4c3c21 ; N#Cc1ccc2c(c1)c1ccccc1n2-c1ccccc1-c1ccccc1-n1c2ccccc2c2ccc3sc4ccccc4c3c21 ; N#Cc1ccc2c(c1)c1ccccc1n2-c1ccccc1-c1ccccc1-n1c2ccccc2c2ccc3c4ccccc4oc3c21 ; N#Cc1ccc2c(c1)c1ccccc1n2-c1ccccc1-c1ccccc1-n1c2ccccc2c2ccc3c4ccccc4sc3c21]

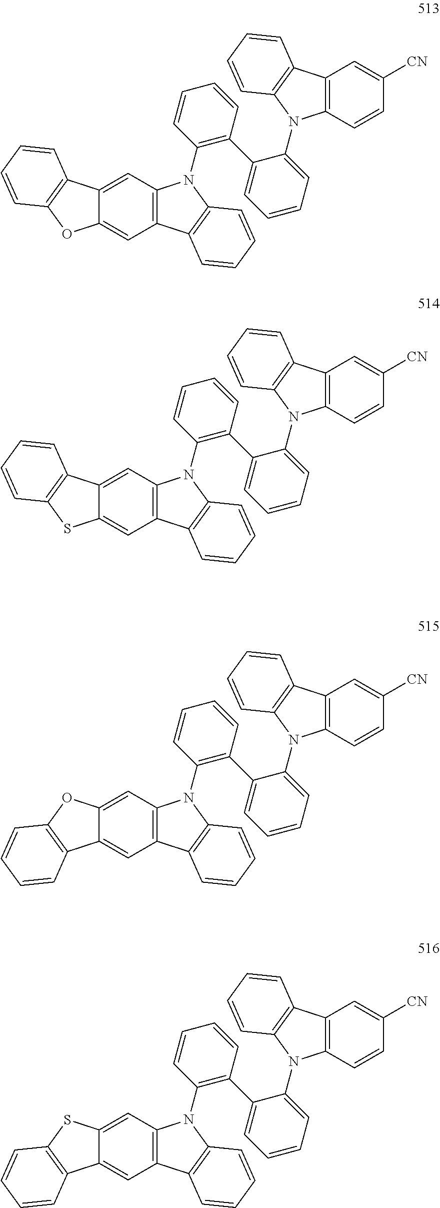 [SMILES: N#Cc1ccc2c(c1)c1ccccc1n2-c1ccccc1-c1ccccc1-n1c2ccccc2c2cc3oc4ccccc4c3cc21 ; N#Cc1ccc2c(c1)c1ccccc1n2-c1ccccc1-c1ccccc1-n1c2ccccc2c2cc3sc4ccccc4c3cc21 ; N#Cc1ccc2c(c1)c1ccccc1n2-c1ccccc1-c1ccccc1-n1c2ccccc2c2cc3c(cc21)oc1ccccc13 ; N#Cc1ccc2c(c1)c1ccccc1n2-c1ccccc1-c1ccccc1-n1c2ccccc2c2cc3c(cc21)sc1ccccc13]

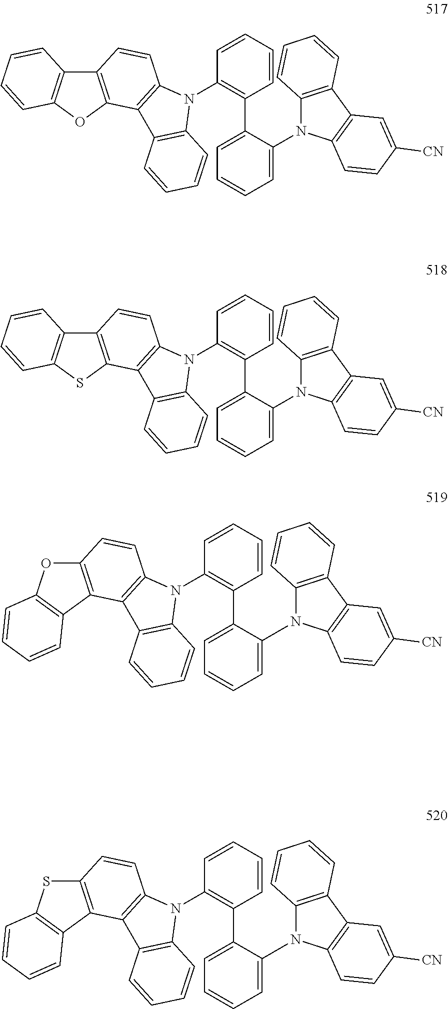 [SMILES: N#Cc1ccc2c(c1)c1ccccc1n2-c1ccccc1-c1ccccc1-n1c2ccccc2c2c3oc4ccccc4c3ccc21 ; N#Cc1ccc2c(c1)c1ccccc1n2-c1ccccc1-c1ccccc1-n1c2ccccc2c2c3sc4ccccc4c3ccc21 ; N#Cc1ccc2c(c1)c1ccccc1n2-c1ccccc1-c1ccccc1-n1c2ccccc2c2c3c(ccc21)oc1ccccc13 ; N#Cc1ccc2c(c1)c1ccccc1n2-c1ccccc1-c1ccccc1-n1c2ccccc2c2c3c(ccc21)sc1ccccc13]

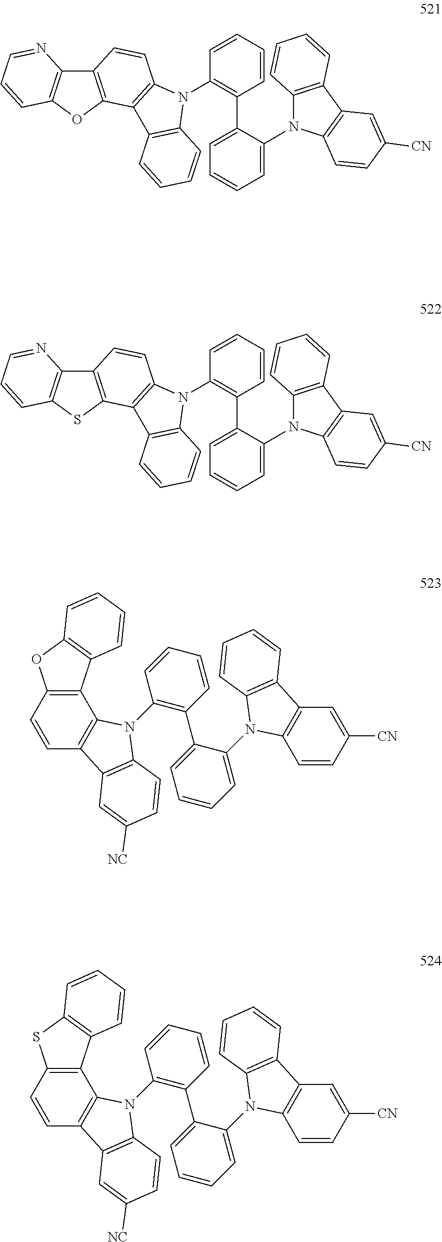 [SMILES: N#Cc1ccc2c(c1)c1ccccc1n2-c1ccccc1-c1ccccc1-n1c2ccccc2c2c3oc4cccnc4c3ccc21 ; N#Cc1ccc2c(c1)c1ccccc1n2-c1ccccc1-c1ccccc1-n1c2ccccc2c2c3sc4cccnc4c3ccc21 ; N#Cc1ccc2c(c1)c1ccccc1n2-c1ccccc1-c1ccccc1-n1c2ccc(C#N)cc2c2ccc3oc4ccccc4c3c21 ; N#Cc1ccc2c(c1)c1ccccc1n2-c1ccccc1-c1ccccc1-n1c2ccc(C#N)cc2c2ccc3sc4ccccc4c3c21]

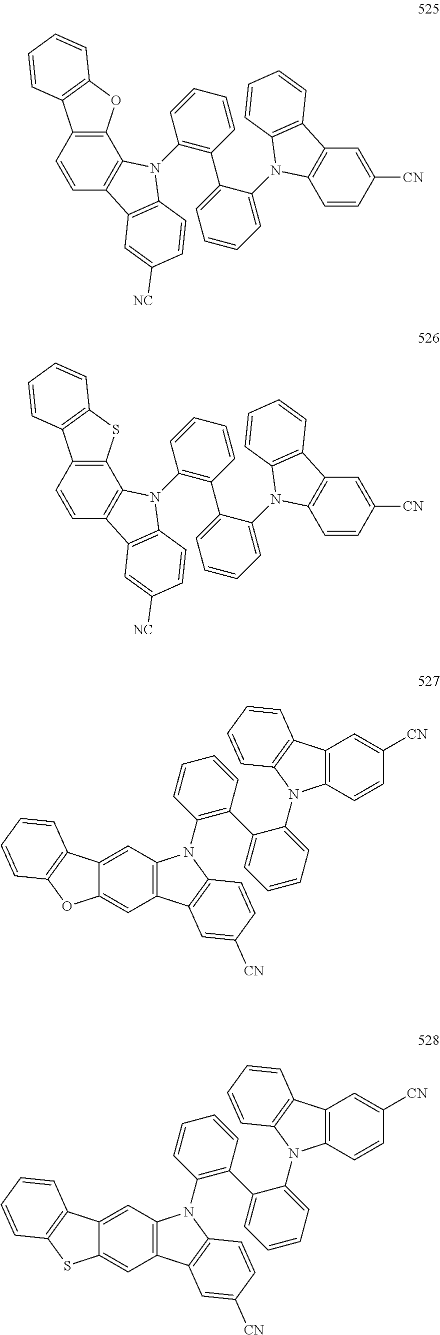 [SMILES: N#Cc1ccc2c(c1)c1ccccc1n2-c1ccccc1-c1ccccc1-n1c2ccc(C#N)cc2c2ccc3c4ccccc4oc3c21 ; N#Cc1ccc2c(c1)c1ccccc1n2-c1ccccc1-c1ccccc1-n1c2ccc(C#N)cc2c2ccc3c4ccccc4sc3c21 ; N#Cc1ccc2c(c1)c1ccccc1n2-c1ccccc1-c1ccccc1-n1c2ccc(C#N)cc2c2cc3oc4ccccc4c3cc21 ; N#Cc1ccc2c(c1)c1ccccc1n2-c1ccccc1-c1ccccc1-n1c2ccc(C#N)cc2c2cc3sc4ccccc4c3cc21]

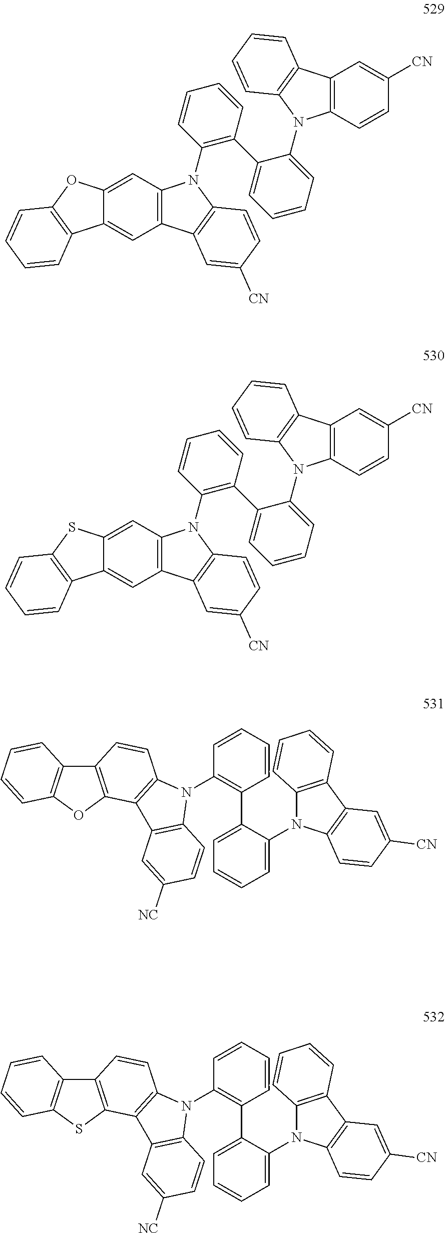 [SMILES: N#Cc1ccc2c(c1)c1ccccc1n2-c1ccccc1-c1ccccc1-n1c2ccc(C#N)cc2c2cc3c(cc21)oc1ccccc13 ; N#Cc1ccc2c(c1)c1ccccc1n2-c1ccccc1-c1ccccc1-n1c2ccc(C#N)cc2c2cc3c(cc21)sc1ccccc13 ; N#Cc1ccc2c(c1)c1ccccc1n2-c1ccccc1-c1ccccc1-n1c2ccc(C#N)cc2c2c3oc4ccccc4c3ccc21 ; N#Cc1ccc2c(c1)c1ccccc1n2-c1ccccc1-c1ccccc1-n1c2ccc(C#N)cc2c2c3sc4ccccc4c3ccc21]

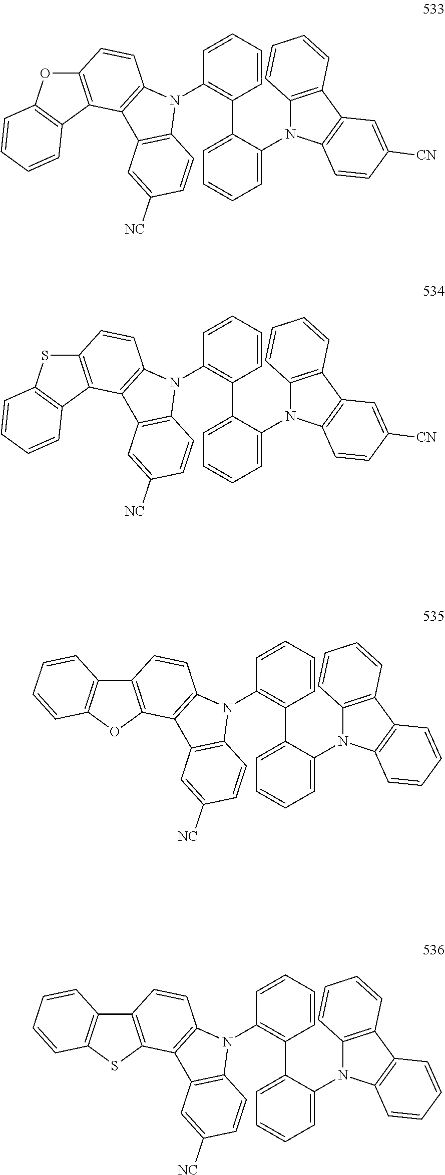 [SMILES: N#Cc1ccc2c(c1)c1ccccc1n2-c1ccccc1-c1ccccc1-n1c2ccc(C#N)cc2c2c3c(ccc21)oc1ccccc13 ; N#Cc1ccc2c(c1)c1ccccc1n2-c1ccccc1-c1ccccc1-n1c2ccc(C#N)cc2c2c3c(ccc21)sc1ccccc13 ; N#Cc1ccc2c(c1)c1c3oc4ccccc4c3ccc1n2-c1ccccc1-c1ccccc1-n1c2ccccc2c2ccccc21 ; N#Cc1ccc2c(c1)c1c3sc4ccccc4c3ccc1n2-c1ccccc1-c1ccccc1-n1c2ccccc2c2ccccc21]

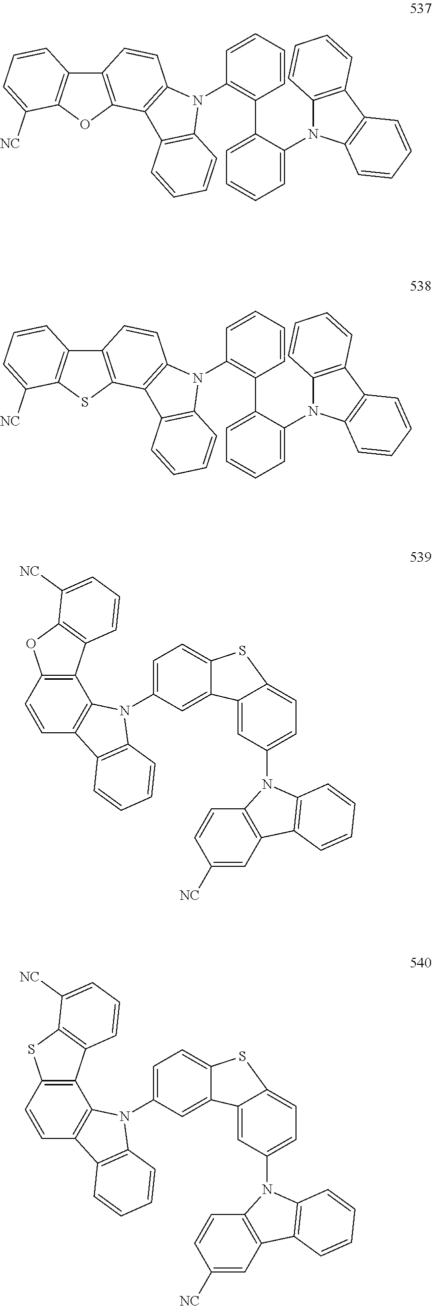 [SMILES: N#Cc1cccc2c1oc1c2ccc2c1c1ccccc1n2-c1ccccc1-c1ccccc1-n1c2ccccc2c2ccccc21 ; N#Cc1cccc2c1sc1c2ccc2c1c1ccccc1n2-c1ccccc1-c1ccccc1-n1c2ccccc2c2ccccc21 ; N#Cc1ccc2c(c1)c1ccccc1n2-c1ccc2sc3ccc(-n4c5ccccc5c5ccc6oc7c(C#N)cccc7c6c54)cc3c2c1 ; N#Cc1ccc2c(c1)c1ccccc1n2-c1ccc2sc3ccc(-n4c5ccccc5c5ccc6sc7c(C#N)cccc7c6c54)cc3c2c1]

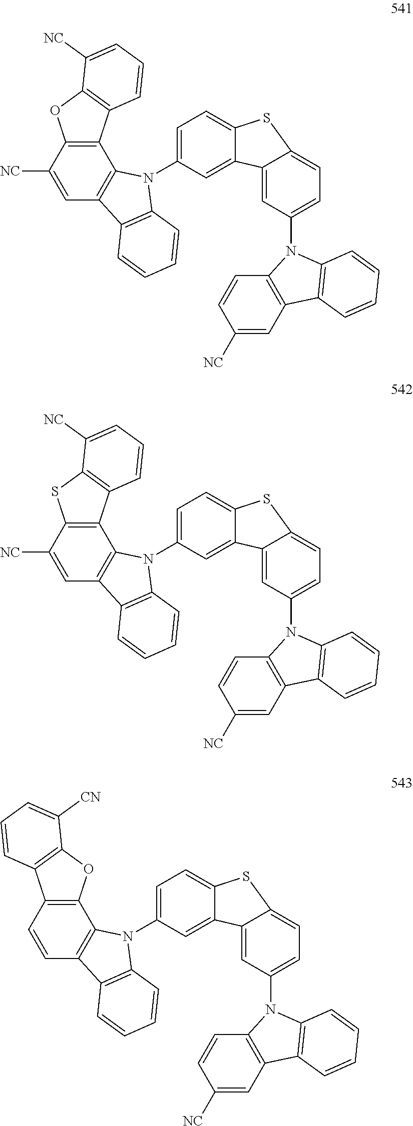 [SMILES: N#Cc1ccc2c(c1)c1ccccc1n2-c1ccc2sc3ccc(-n4c5ccccc5c5cc(C#N)c6oc7c(C#N)cccc7c6c54)cc3c2c1 ; N#Cc1ccc2c(c1)c1ccccc1n2-c1ccc2sc3ccc(-n4c5ccccc5c5cc(C#N)c6sc7c(C#N)cccc7c6c54)cc3c2c1 ; N#Cc1ccc2c(c1)c1ccccc1n2-c1ccc2sc3ccc(-n4c5ccccc5c5ccc6c7cccc(C#N)c7oc6c54)cc3c2c1]

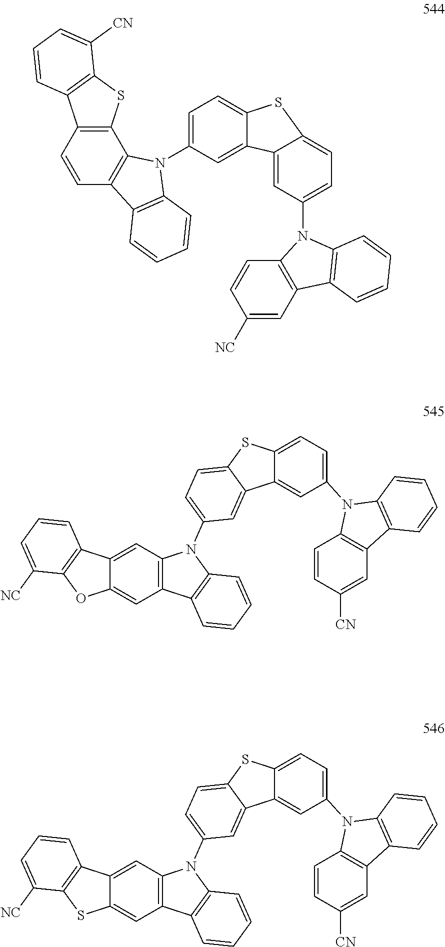 [SMILES: N#Cc1ccc2c(c1)c1ccccc1n2-c1ccc2sc3ccc(-n4c5ccccc5c5ccc6c7cccc(C#N)c7sc6c54)cc3c2c1 ; N#Cc1ccc2c(c1)c1ccccc1n2-c1ccc2sc3ccc(-n4c5ccccc5c5cc6oc7c(C#N)cccc7c6cc54)cc3c2c1 ; N#Cc1ccc2c(c1)c1ccccc1n2-c1ccc2sc3ccc(-n4c5ccccc5c5cc6sc7c(C#N)cccc7c6cc54)cc3c2c1]

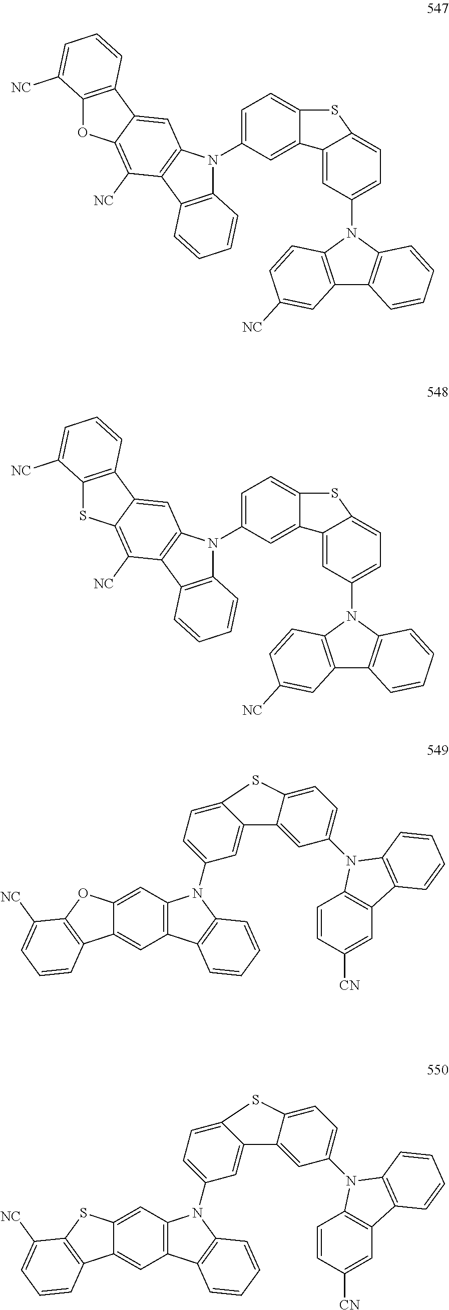 [SMILES: N#Cc1ccc2c(c1)c1ccccc1n2-c1ccc2sc3ccc(-n4c5ccccc5c5c(C#N)c6oc7c(C#N)cccc7c6cc54)cc3c2c1 ; N#Cc1ccc2c(c1)c1ccccc1n2-c1ccc2sc3ccc(-n4c5ccccc5c5c(C#N)c6sc7c(C#N)cccc7c6cc54)cc3c2c1 ; N#Cc1ccc2c(c1)c1ccccc1n2-c1ccc2sc3ccc(-n4c5ccccc5c5cc6c(cc54)oc4c(C#N)cccc46)cc3c2c1 ; N#Cc1ccc2c(c1)c1ccccc1n2-c1ccc2sc3ccc(-n4c5ccccc5c5cc6c(cc54)sc4c(C#N)cccc46)cc3c2c1]

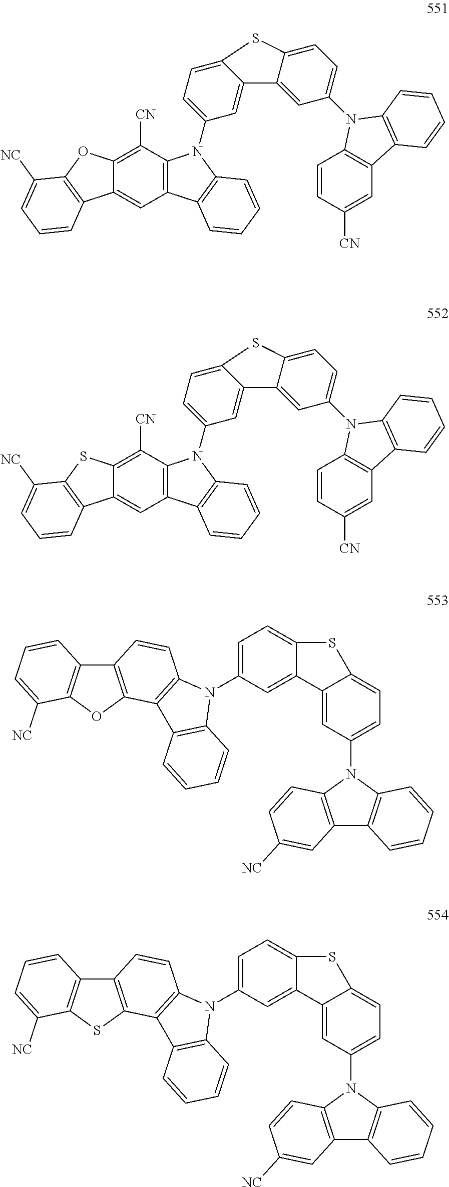 [SMILES: N#Cc1ccc2c(c1)c1ccccc1n2-c1ccc2sc3ccc(-n4c5ccccc5c5cc6c(oc7c(C#N)cccc76)c(C#N)c54)cc3c2c1 ; N#Cc1ccc2c(c1)c1ccccc1n2-c1ccc2sc3ccc(-n4c5ccccc5c5cc6c(sc7c(C#N)cccc76)c(C#N)c54)cc3c2c1 ; N#Cc1ccc2c(c1)c1ccccc1n2-c1ccc2sc3ccc(-n4c5ccccc5c5c6oc7c(C#N)cccc7c6ccc54)cc3c2c1 ; N#Cc1ccc2c(c1)c1ccccc1n2-c1ccc2sc3ccc(-n4c5ccccc5c5c6sc7c(C#N)cccc7c6ccc54)cc3c2c1]

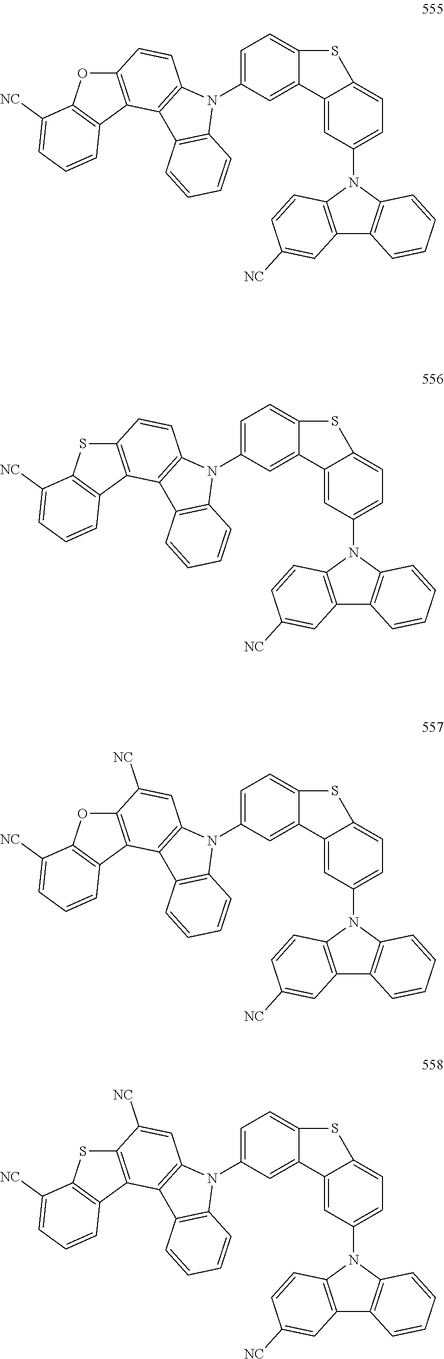 [SMILES: N#Cc1ccc2c(c1)c1ccccc1n2-c1ccc2sc3ccc(-n4c5ccccc5c5c6c(ccc54)oc4c(C#N)cccc46)cc3c2c1 ; N#Cc1ccc2c(c1)c1ccccc1n2-c1ccc2sc3ccc(-n4c5ccccc5c5c6c(ccc54)sc4c(C#N)cccc46)cc3c2c1 ; N#Cc1ccc2c(c1)c1ccccc1n2-c1ccc2sc3ccc(-n4c5ccccc5c5c6c(oc7c(C#N)cccc76)c(C#N)cc54)cc3c2c1 ; N#Cc1ccc2c(c1)c1ccccc1n2-c1ccc2sc3ccc(-n4c5ccccc5c5c6c(sc7c(C#N)cccc76)c(C#N)cc54)cc3c2c1]

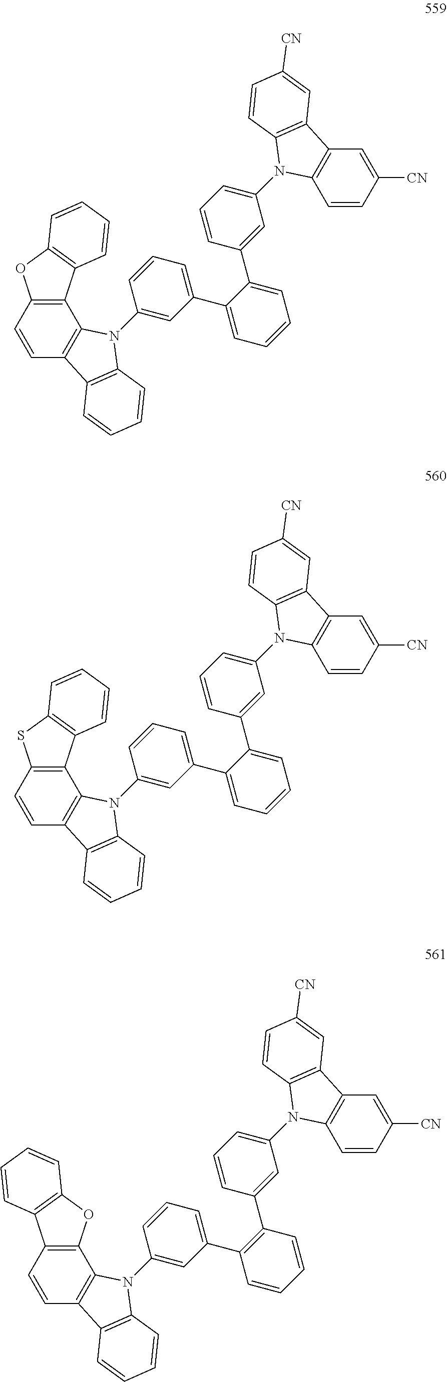 [SMILES: N#Cc1ccc2c(c1)c1cc(C#N)ccc1n2-c1cccc(-c2ccccc2-c2cccc(-n3c4ccccc4c4ccc5oc6ccccc6c5c43)c2)c1 ; N#Cc1ccc2c(c1)c1cc(C#N)ccc1n2-c1cccc(-c2ccccc2-c2cccc(-n3c4ccccc4c4ccc5sc6ccccc6c5c43)c2)c1 ; N#Cc1ccc2c(c1)c1cc(C#N)ccc1n2-c1cccc(-c2ccccc2-c2cccc(-n3c4ccccc4c4ccc5c6ccccc6oc5c43)c2)c1]

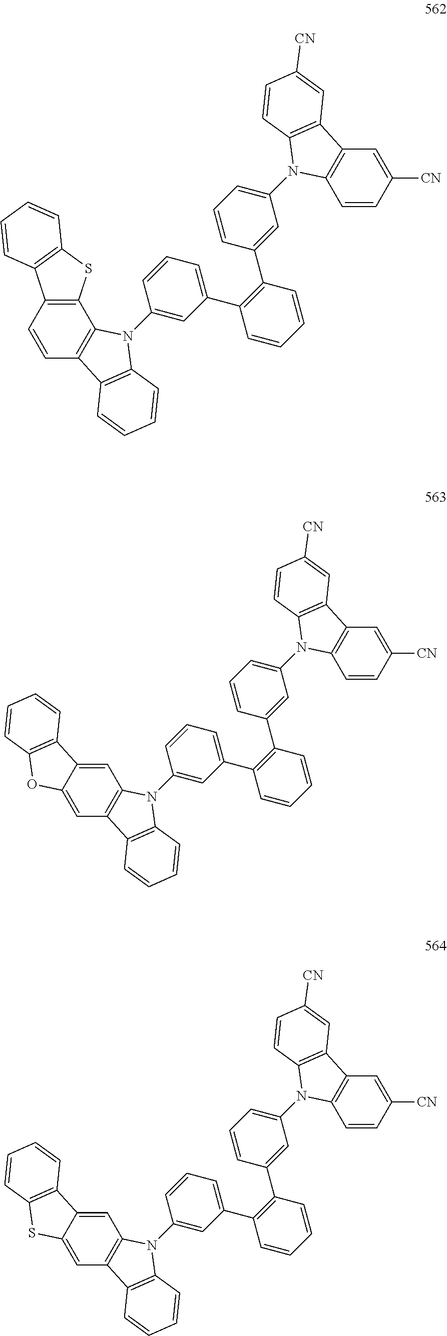 [SMILES: N#Cc1ccc2c(c1)c1cc(C#N)ccc1n2-c1cccc(-c2ccccc2-c2cccc(-n3c4ccccc4c4ccc5c6ccccc6sc5c43)c2)c1 ; N#Cc1ccc2c(c1)c1cc(C#N)ccc1n2-c1cccc(-c2ccccc2-c2cccc(-n3c4ccccc4c4cc5oc6ccccc6c5cc43)c2)c1 ; N#Cc1ccc2c(c1)c1cc(C#N)ccc1n2-c1cccc(-c2ccccc2-c2cccc(-n3c4ccccc4c4cc5sc6ccccc6c5cc43)c2)c1]

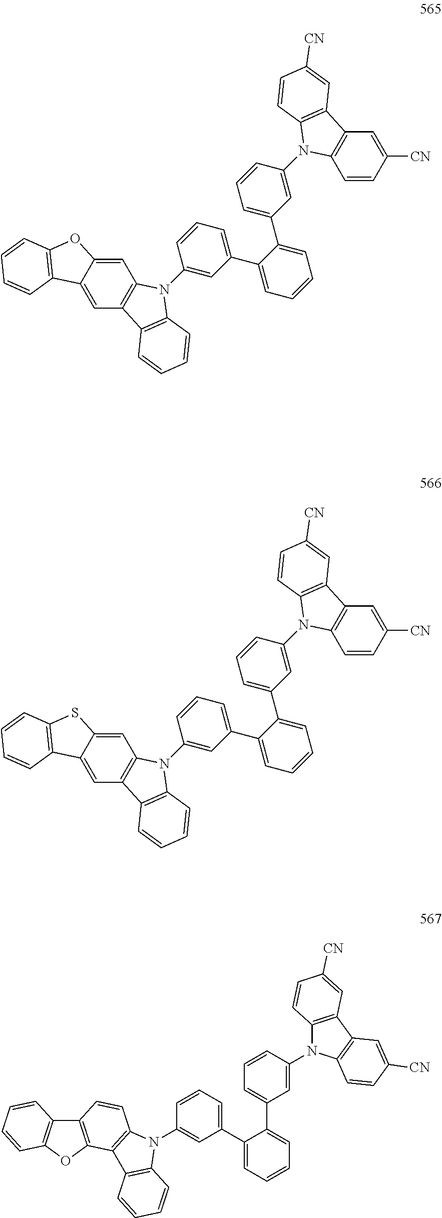 [SMILES: N#Cc1ccc2c(c1)c1cc(C#N)ccc1n2-c1cccc(-c2ccccc2-c2cccc(-n3c4ccccc4c4cc5c(cc43)oc3ccccc35)c2)c1 ; N#Cc1ccc2c(c1)c1cc(C#N)ccc1n2-c1cccc(-c2ccccc2-c2cccc(-n3c4ccccc4c4cc5c(cc43)sc3ccccc35)c2)c1 ; N#Cc1ccc2c(c1)c1cc(C#N)ccc1n2-c1cccc(-c2ccccc2-c2cccc(-n3c4ccccc4c4c5oc6ccccc6c5ccc43)c2)c1]

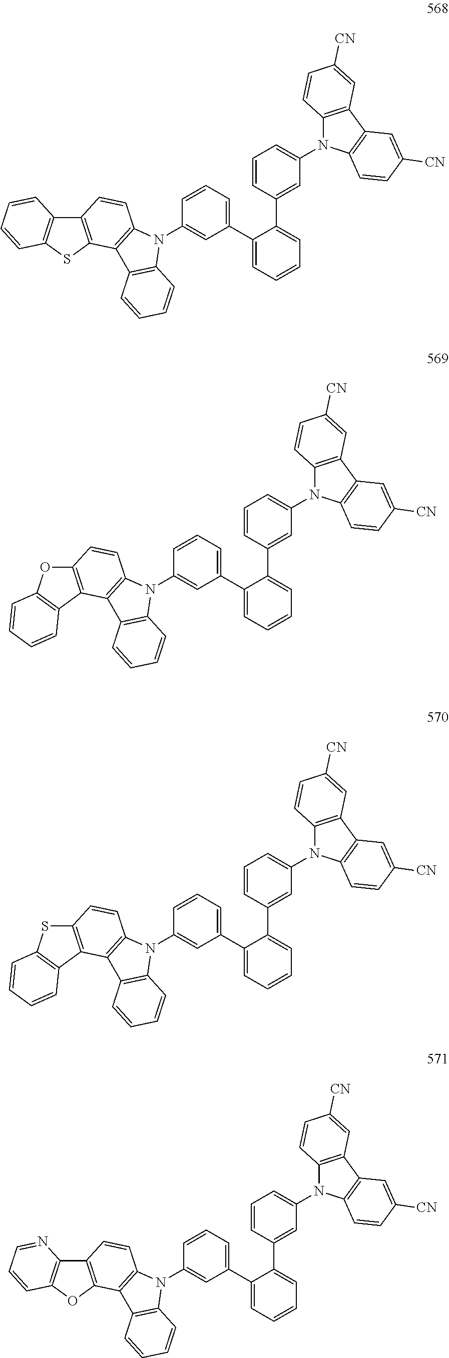 [SMILES: N#Cc1ccc2c(c1)c1cc(C#N)ccc1n2-c1cccc(-c2ccccc2-c2cccc(-n3c4ccccc4c4c5sc6ccccc6c5ccc43)c2)c1 ; N#Cc1ccc2c(c1)c1cc(C#N)ccc1n2-c1cccc(-c2ccccc2-c2cccc(-n3c4ccccc4c4c5c(ccc43)oc3ccccc35)c2)c1 ; N#Cc1ccc2c(c1)c1cc(C#N)ccc1n2-c1cccc(-c2ccccc2-c2cccc(-n3c4ccccc4c4c5c(ccc43)sc3ccccc35)c2)c1 ; N#Cc1ccc2c(c1)c1cc(C#N)ccc1n2-c1cccc(-c2ccccc2-c2cccc(-n3c4ccccc4c4c5oc6cccnc6c5ccc43)c2)c1]

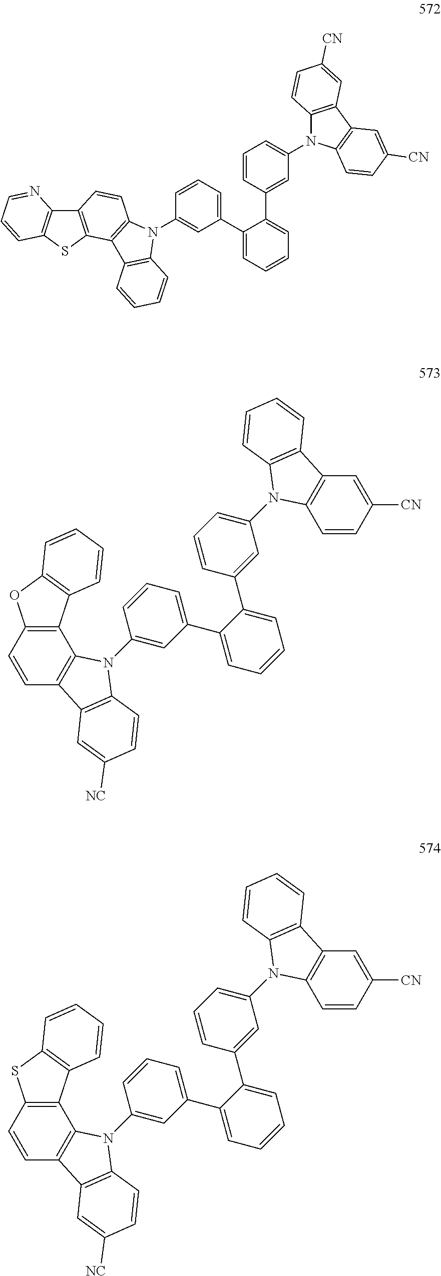 [SMILES: N#Cc1ccc2c(c1)c1cc(C#N)ccc1n2-c1cccc(-c2ccccc2-c2cccc(-n3c4ccccc4c4c5sc6cccnc6c5ccc43)c2)c1 ; N#Cc1ccc2c(c1)c1ccccc1n2-c1cccc(-c2ccccc2-c2cccc(-n3c4ccc(C#N)cc4c4ccc5oc6ccccc6c5c43)c2)c1 ; N#Cc1ccc2c(c1)c1ccccc1n2-c1cccc(-c2ccccc2-c2cccc(-n3c4ccc(C#N)cc4c4ccc5sc6ccccc6c5c43)c2)c1]

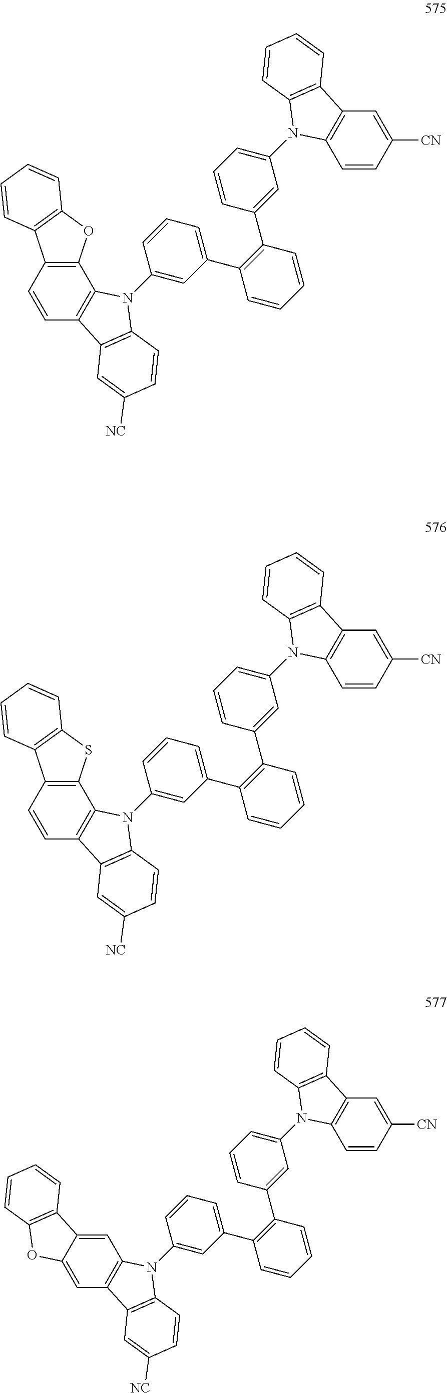 [SMILES: N#Cc1ccc2c(c1)c1ccccc1n2-c1cccc(-c2ccccc2-c2cccc(-n3c4ccc(C#N)cc4c4ccc5c6ccccc6oc5c43)c2)c1 ; N#Cc1ccc2c(c1)c1ccccc1n2-c1cccc(-c2ccccc2-c2cccc(-n3c4ccc(C#N)cc4c4ccc5c6ccccc6sc5c43)c2)c1 ; N#Cc1ccc2c(c1)c1ccccc1n2-c1cccc(-c2ccccc2-c2cccc(-n3c4ccc(C#N)cc4c4cc5oc6ccccc6c5cc43)c2)c1]

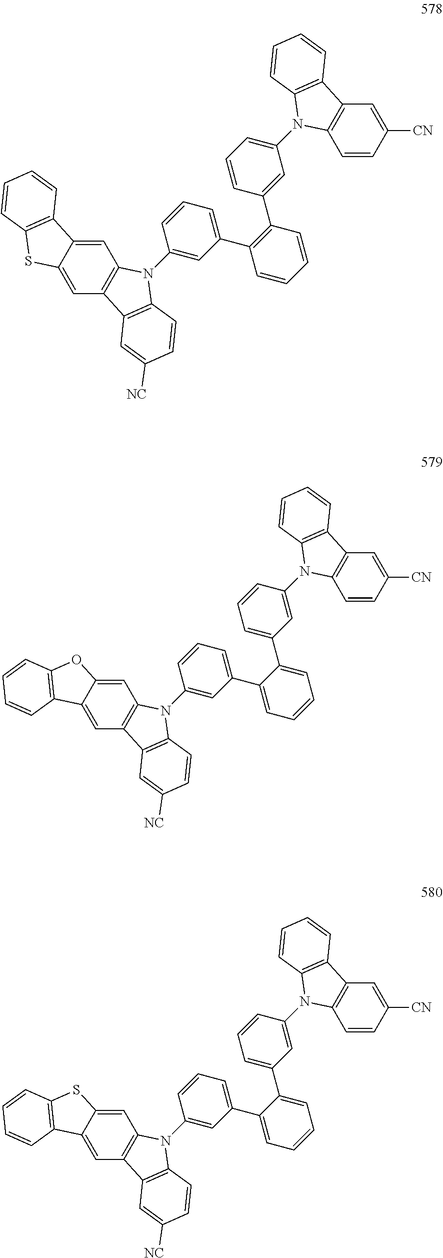 [SMILES: N#Cc1ccc2c(c1)c1ccccc1n2-c1cccc(-c2ccccc2-c2cccc(-n3c4ccc(C#N)cc4c4cc5sc6ccccc6c5cc43)c2)c1 ; N#Cc1ccc2c(c1)c1ccccc1n2-c1cccc(-c2ccccc2-c2cccc(-n3c4ccc(C#N)cc4c4cc5c(cc43)oc3ccccc35)c2)c1 ; N#Cc1ccc2c(c1)c1ccccc1n2-c1cccc(-c2ccccc2-c2cccc(-n3c4ccc(C#N)cc4c4cc5c(cc43)sc3ccccc35)c2)c1]

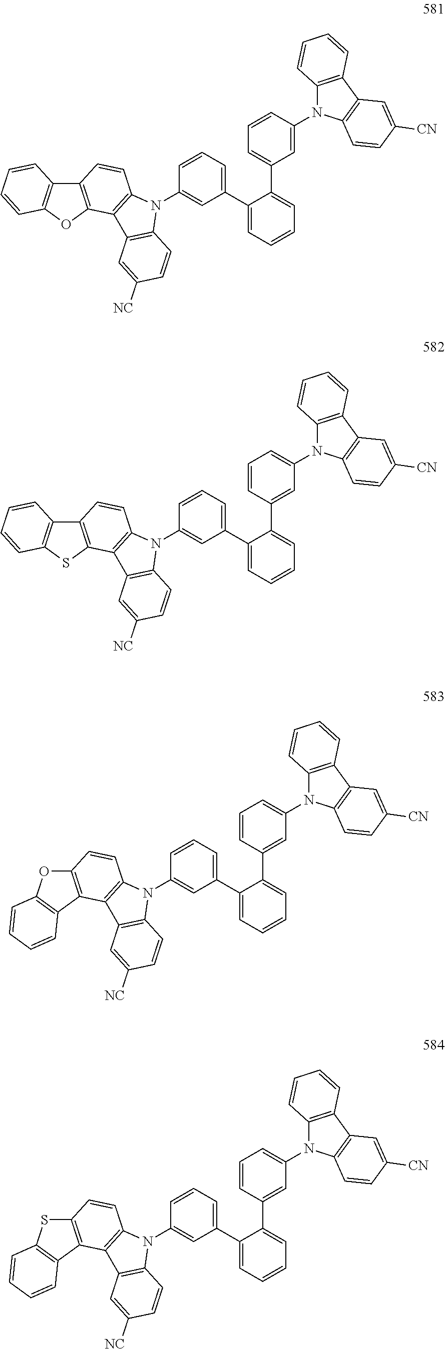 [SMILES: N#Cc1ccc2c(c1)c1ccccc1n2-c1cccc(-c2ccccc2-c2cccc(-n3c4ccc(C#N)cc4c4c5oc6ccccc6c5ccc43)c2)c1 ; N#Cc1ccc2c(c1)c1ccccc1n2-c1cccc(-c2ccccc2-c2cccc(-n3c4ccc(C#N)cc4c4c5sc6ccccc6c5ccc43)c2)c1 ; N#Cc1ccc2c(c1)c1ccccc1n2-c1cccc(-c2ccccc2-c2cccc(-n3c4ccc(C#N)cc4c4c5c(ccc43)oc3ccccc35)c2)c1 ; N#Cc1ccc2c(c1)c1ccccc1n2-c1cccc(-c2ccccc2-c2cccc(-n3c4ccc(C#N)cc4c4c5c(ccc43)sc3ccccc35)c2)c1]

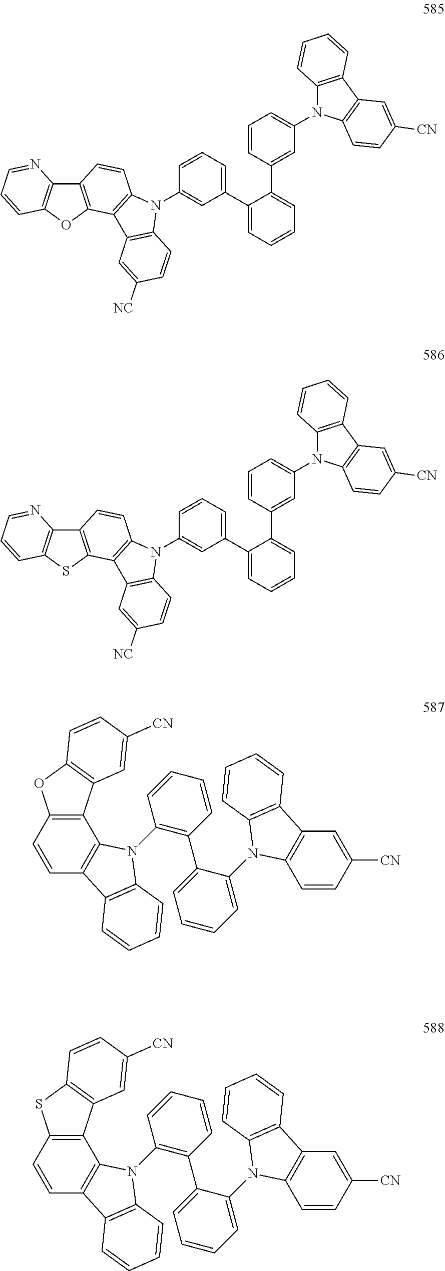 [SMILES: N#Cc1ccc2c(c1)c1ccccc1n2-c1cccc(-c2ccccc2-c2cccc(-n3c4ccc(C#N)cc4c4c5oc6cccnc6c5ccc43)c2)c1 ; N#Cc1ccc2c(c1)c1ccccc1n2-c1cccc(-c2ccccc2-c2cccc(-n3c4ccc(C#N)cc4c4c5sc6cccnc6c5ccc43)c2)c1 ; N#Cc1ccc2oc3ccc4c5ccccc5n(-c5ccccc5-c5ccccc5-n5c6ccccc6c6cc(C#N)ccc65)c4c3c2c1 ; N#Cc1ccc2sc3ccc4c5ccccc5n(-c5ccccc5-c5ccccc5-n5c6ccccc6c6cc(C#N)ccc65)c4c3c2c1]

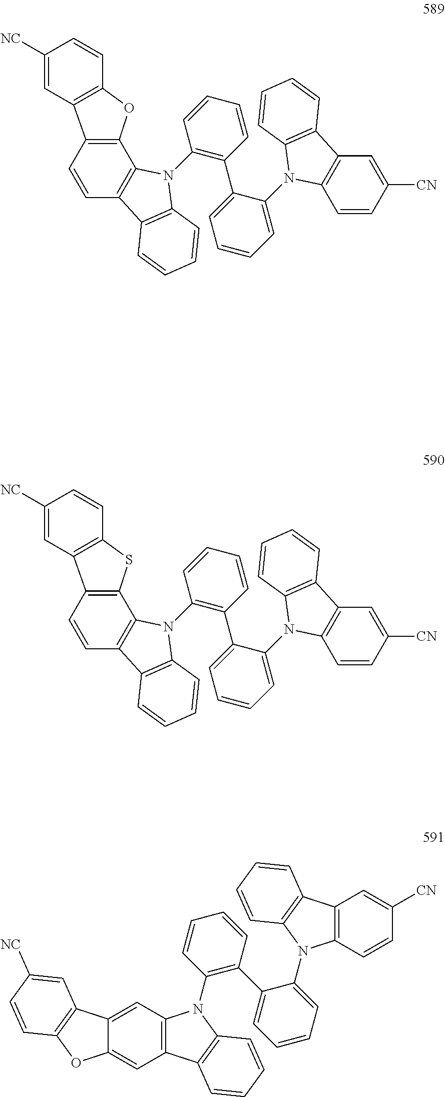 [SMILES: N#Cc1ccc2oc3c(ccc4c5ccccc5n(-c5ccccc5-c5ccccc5-n5c6ccccc6c6cc(C#N)ccc65)c43)c2c1 ; N#Cc1ccc2sc3c(ccc4c5ccccc5n(-c5ccccc5-c5ccccc5-n5c6ccccc6c6cc(C#N)ccc65)c43)c2c1 ; N#Cc1ccc2oc3cc4c5ccccc5n(-c5ccccc5-c5ccccc5-n5c6ccccc6c6cc(C#N)ccc65)c4cc3c2c1]

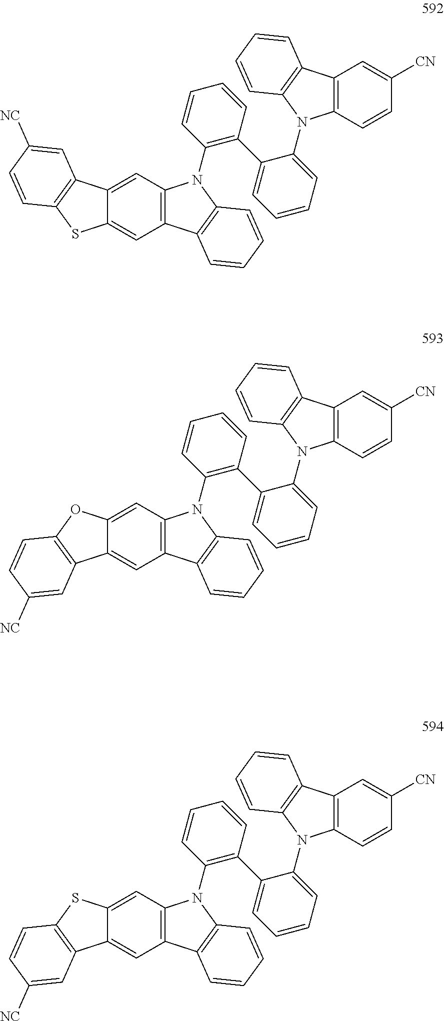 [SMILES: N#Cc1ccc2sc3cc4c5ccccc5n(-c5ccccc5-c5ccccc5-n5c6ccccc6c6cc(C#N)ccc65)c4cc3c2c1 ; N#Cc1ccc2oc3cc4c(cc3c2c1)c1ccccc1n4-c1ccccc1-c1ccccc1-n1c2ccccc2c2cc(C#N)ccc21 ; N#Cc1ccc2sc3cc4c(cc3c2c1)c1ccccc1n4-c1ccccc1-c1ccccc1-n1c2ccccc2c2cc(C#N)ccc21]

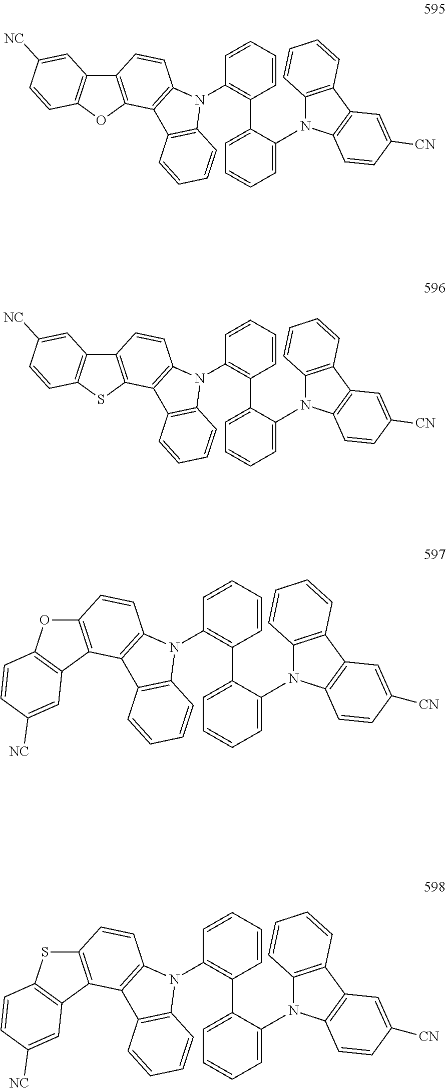 [SMILES: N#Cc1ccc2oc3c(ccc4c3c3ccccc3n4-c3ccccc3-c3ccccc3-n3c4ccccc4c4cc(C#N)ccc43)c2c1 ; N#Cc1ccc2sc3c(ccc4c3c3ccccc3n4-c3ccccc3-c3ccccc3-n3c4ccccc4c4cc(C#N)ccc43)c2c1 ; N#Cc1ccc2oc3ccc4c(c5ccccc5n4-c4ccccc4-c4ccccc4-n4c5ccccc5c5cc(C#N)ccc54)c3c2c1 ; N#Cc1ccc2sc3ccc4c(c5ccccc5n4-c4ccccc4-c4ccccc4-n4c5ccccc5c5cc(C#N)ccc54)c3c2c1]

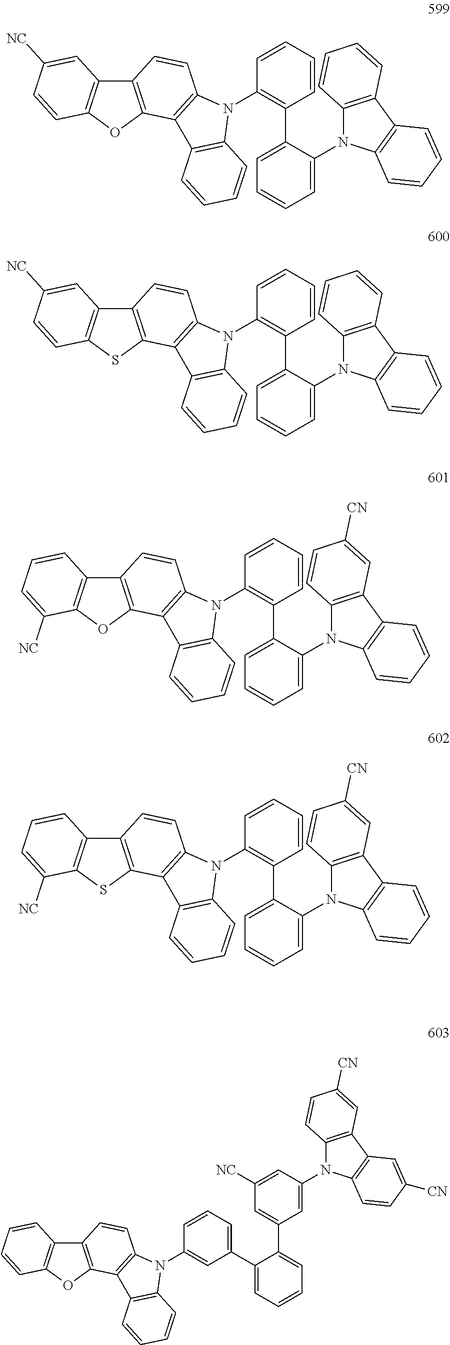 [SMILES: N#Cc1ccc2oc3c(ccc4c3c3ccccc3n4-c3ccccc3-c3ccccc3-n3c4ccccc4c4ccccc43)c2c1 ; N#Cc1ccc2sc3c(ccc4c3c3ccccc3n4-c3ccccc3-c3ccccc3-n3c4ccccc4c4ccccc43)c2c1 ; N#Cc1ccc2c(c1)c1ccccc1n2-c1ccccc1-c1ccccc1-n1c2ccccc2c2c3oc4c(C#N)cccc4c3ccc21 ; N#Cc1ccc2c(c1)c1ccccc1n2-c1ccccc1-c1ccccc1-n1c2ccccc2c2c3sc4c(C#N)cccc4c3ccc21 ; N#Cc1cc(-c2ccccc2-c2cccc(-n3c4ccccc4c4c5oc6ccccc6c5ccc43)c2)cc(-n2c3ccc(C#N)cc3c3cc(C#N)ccc32)c1]

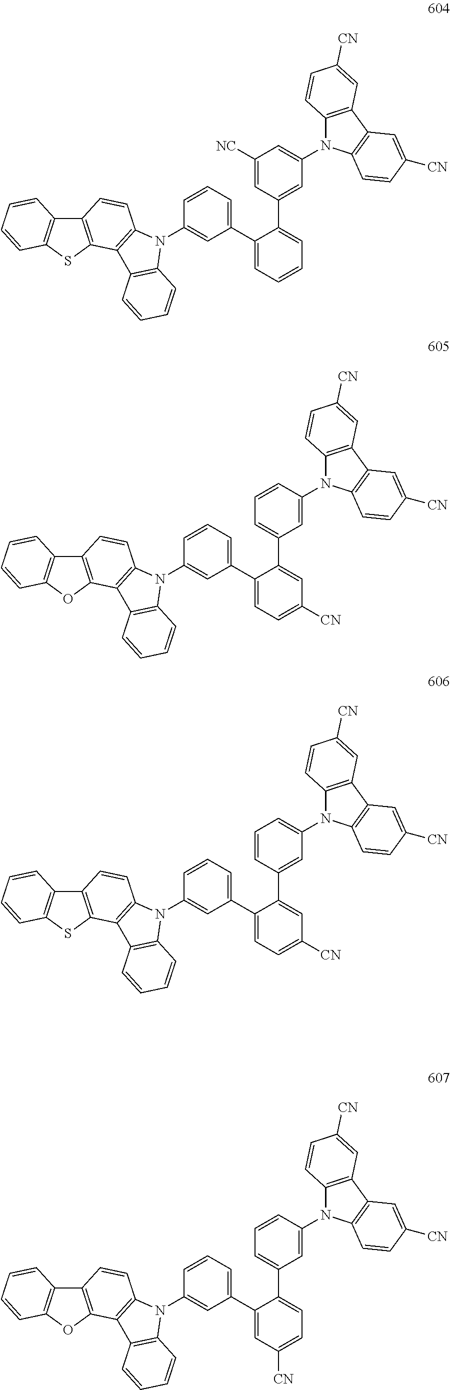 [SMILES: N#Cc1cc(-c2ccccc2-c2cccc(-n3c4ccccc4c4c5sc6ccccc6c5ccc43)c2)cc(-n2c3ccc(C#N)cc3c3cc(C#N)ccc32)c1 ; N#Cc1ccc(-c2cccc(-n3c4ccccc4c4c5oc6ccccc6c5ccc43)c2)c(-c2cccc(-n3c4ccc(C#N)cc4c4cc(C#N)ccc43)c2)c1 ; N#Cc1ccc(-c2cccc(-n3c4ccccc4c4c5sc6ccccc6c5ccc43)c2)c(-c2cccc(-n3c4ccc(C#N)cc4c4cc(C#N)ccc43)c2)c1 ; N#Cc1ccc(-c2cccc(-n3c4ccc(C#N)cc4c4cc(C#N)ccc43)c2)c(-c2cccc(-n3c4ccccc4c4c5oc6ccccc6c5ccc43)c2)c1]

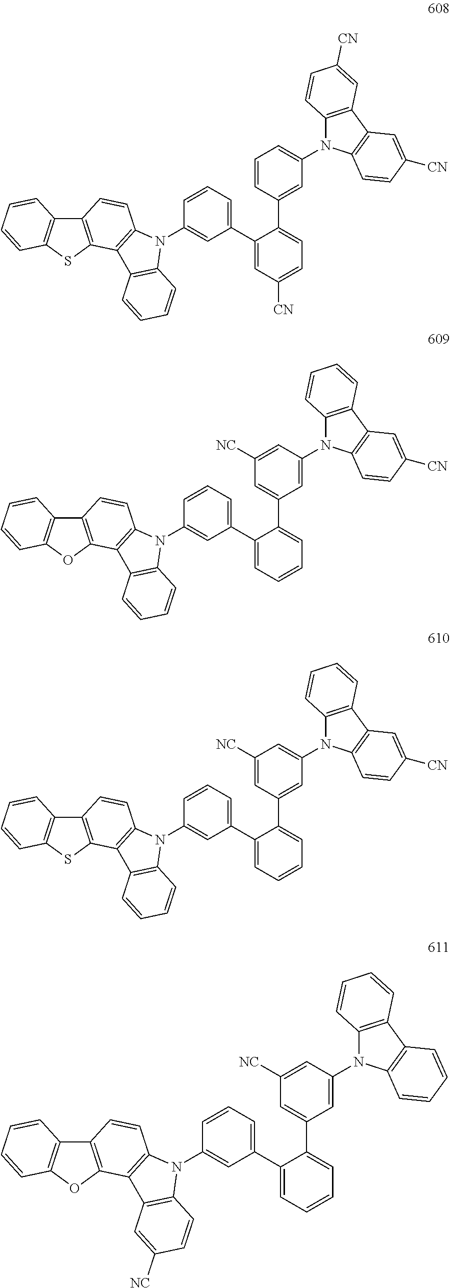 [SMILES: N#Cc1ccc(-c2cccc(-n3c4ccc(C#N)cc4c4cc(C#N)ccc43)c2)c(-c2cccc(-n3c4ccccc4c4c5sc6ccccc6c5ccc43)c2)c1 ; N#Cc1cc(-c2ccccc2-c2cccc(-n3c4ccccc4c4c5oc6ccccc6c5ccc43)c2)cc(-n2c3ccccc3c3cc(C#N)ccc32)c1 ; N#Cc1cc(-c2ccccc2-c2cccc(-n3c4ccccc4c4c5sc6ccccc6c5ccc43)c2)cc(-n2c3ccccc3c3cc(C#N)ccc32)c1 ; N#Cc1cc(-c2ccccc2-c2cccc(-n3c4ccc(C#N)cc4c4c5oc6ccccc6c5ccc43)c2)cc(-n2c3ccccc3c3ccccc32)c1]

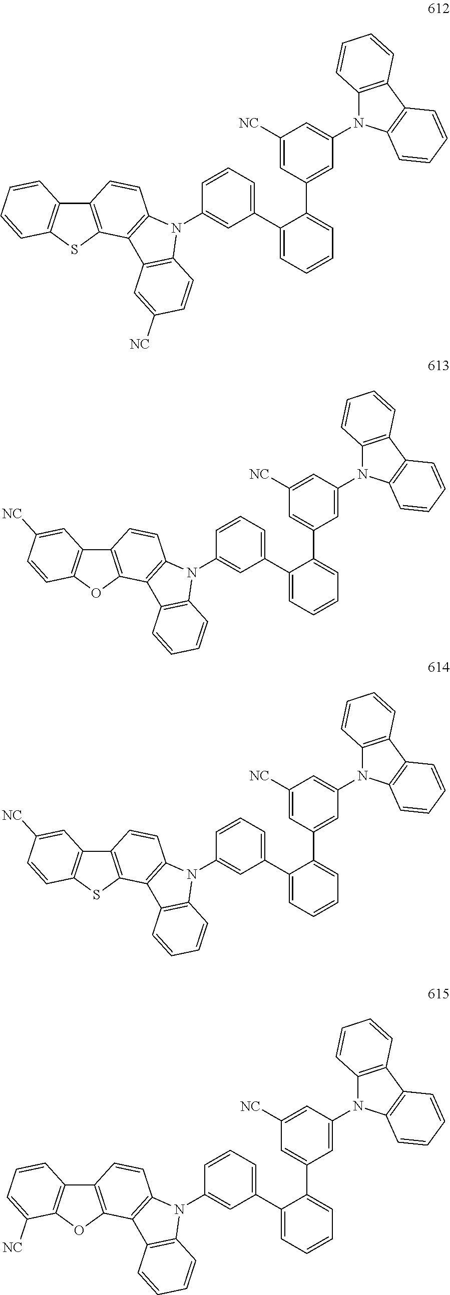 [SMILES: N#Cc1cc(-c2ccccc2-c2cccc(-n3c4ccc(C#N)cc4c4c5sc6ccccc6c5ccc43)c2)cc(-n2c3ccccc3c3ccccc32)c1 ; N#Cc1cc(-c2ccccc2-c2cccc(-n3c4ccccc4c4c5oc6ccc(C#N)cc6c5ccc43)c2)cc(-n2c3ccccc3c3ccccc32)c1 ; N#Cc1cc(-c2ccccc2-c2cccc(-n3c4ccccc4c4c5sc6ccc(C#N)cc6c5ccc43)c2)cc(-n2c3ccccc3c3ccccc32)c1 ; N#Cc1cc(-c2ccccc2-c2cccc(-n3c4ccccc4c4c5oc6c(C#N)cccc6c5ccc43)c2)cc(-n2c3ccccc3c3ccccc32)c1]

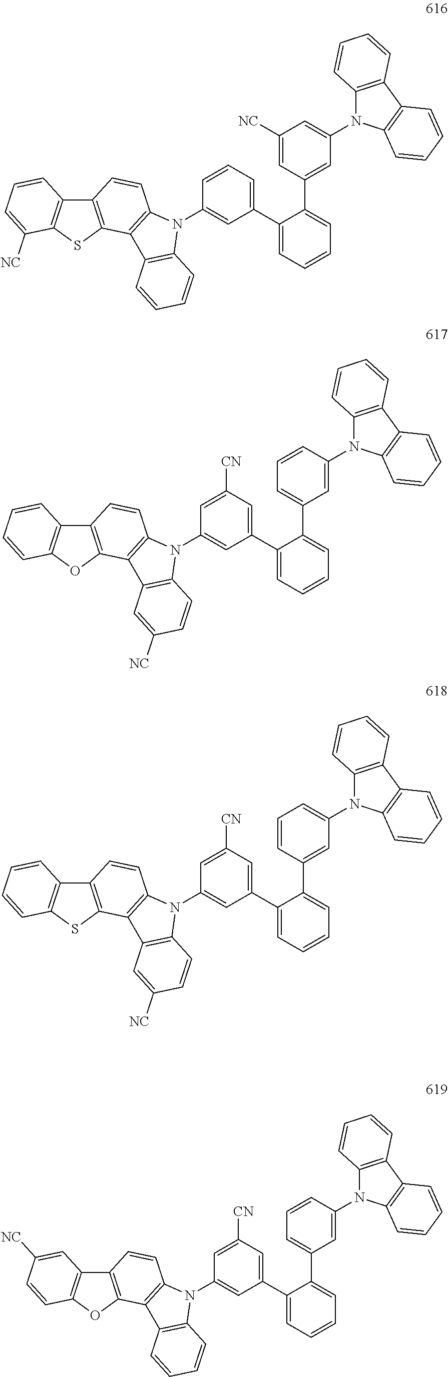 [SMILES: N#Cc1cc(-c2ccccc2-c2cccc(-n3c4ccccc4c4c5sc6c(C#N)cccc6c5ccc43)c2)cc(-n2c3ccccc3c3ccccc32)c1 ; N#Cc1cc(-c2ccccc2-c2cccc(-n3c4ccccc4c4ccccc43)c2)cc(-n2c3ccc(C#N)cc3c3c4oc5ccccc5c4ccc32)c1 ; N#Cc1cc(-c2ccccc2-c2cccc(-n3c4ccccc4c4ccccc43)c2)cc(-n2c3ccc(C#N)cc3c3c4sc5ccccc5c4ccc32)c1 ; N#Cc1cc(-c2ccccc2-c2cccc(-n3c4ccccc4c4ccccc43)c2)cc(-n2c3ccccc3c3c4oc5ccc(C#N)cc5c4ccc32)c1]

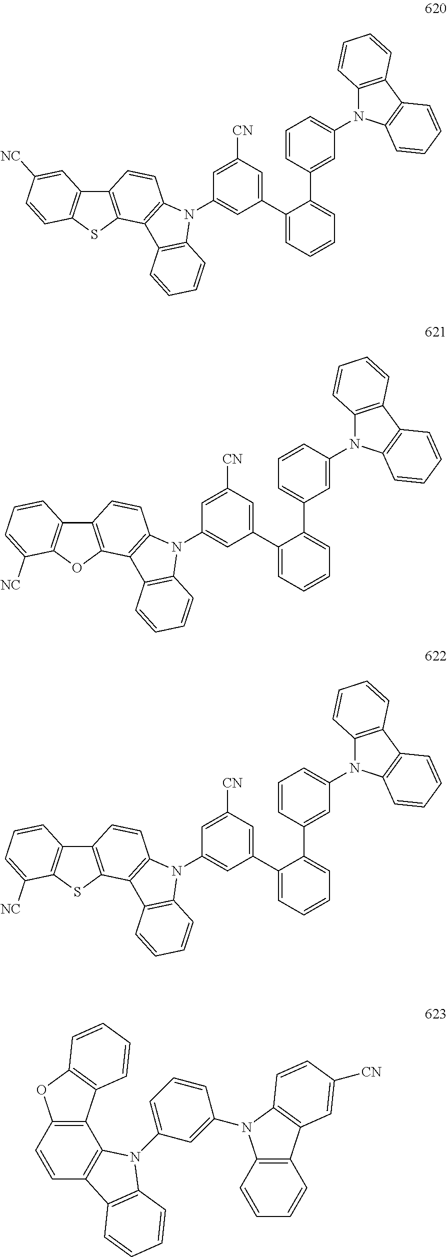 [SMILES: N#Cc1cc(-c2ccccc2-c2cccc(-n3c4ccccc4c4ccccc43)c2)cc(-n2c3ccccc3c3c4sc5ccc(C#N)cc5c4ccc32)c1 ; N#Cc1cc(-c2ccccc2-c2cccc(-n3c4ccccc4c4ccccc43)c2)cc(-n2c3ccccc3c3c4oc5c(C#N)cccc5c4ccc32)c1 ; N#Cc1cc(-c2ccccc2-c2cccc(-n3c4ccccc4c4ccccc43)c2)cc(-n2c3ccccc3c3c4sc5c(C#N)cccc5c4ccc32)c1 ; N#Cc1ccc2c(c1)c1ccccc1n2-c1cccc(-n2c3ccccc3c3ccc4oc5ccccc5c4c32)c1]

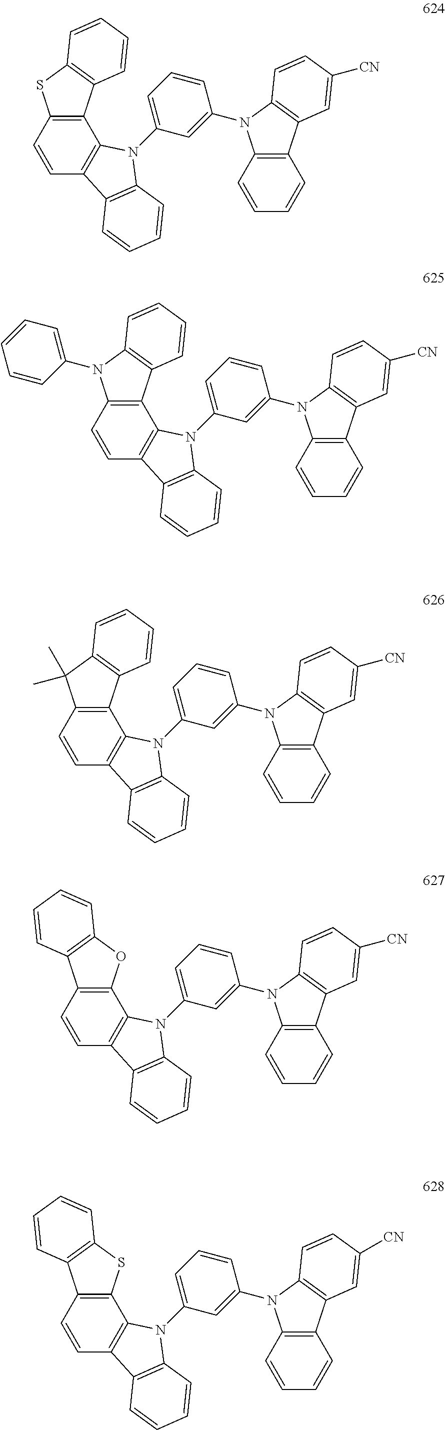 [SMILES: N#Cc1ccc2c(c1)c1ccccc1n2-c1cccc(-n2c3ccccc3c3ccc4sc5ccccc5c4c32)c1 ; N#Cc1ccc2c(c1)c1ccccc1n2-c1cccc(-n2c3ccccc3c3ccc4c(c5ccccc5n4-c4ccccc4)c32)c1 ; CC1(C)c2ccccc2-c2c1ccc1c3ccccc3n(-c3cccc(-n4c5ccccc5c5cc(C#N)ccc54)c3)c21 ; N#Cc1ccc2c(c1)c1ccccc1n2-c1cccc(-n2c3ccccc3c3ccc4c5ccccc5oc4c32)c1 ; N#Cc1ccc2c(c1)c1ccccc1n2-c1cccc(-n2c3ccccc3c3ccc4c5ccccc5sc4c32)c1]

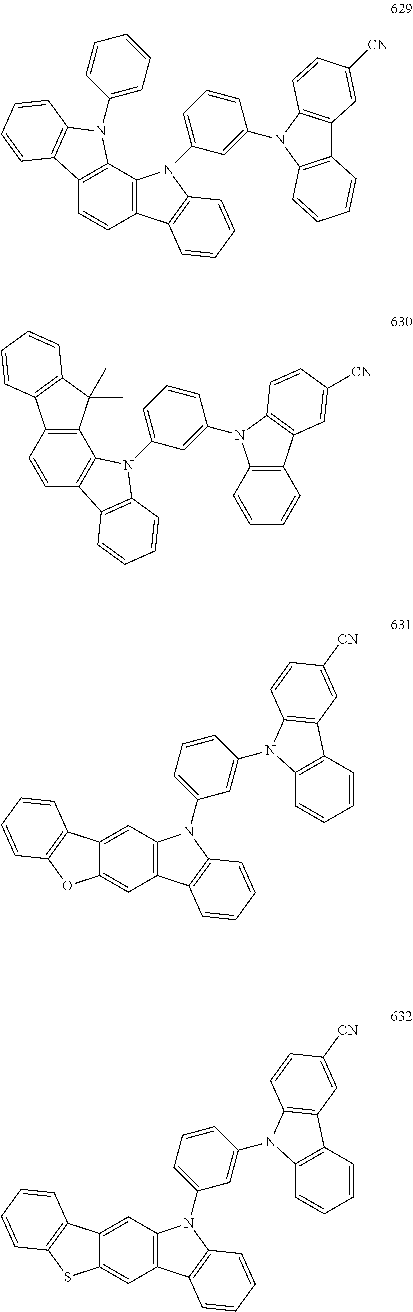 [SMILES: N#Cc1ccc2c(c1)c1ccccc1n2-c1cccc(-n2c3ccccc3c3ccc4c5ccccc5n(-c5ccccc5)c4c32)c1 ; CC1(C)c2ccccc2-c2ccc3c4ccccc4n(-c4cccc(-n5c6ccccc6c6cc(C#N)ccc65)c4)c3c21 ; N#Cc1ccc2c(c1)c1ccccc1n2-c1cccc(-n2c3ccccc3c3cc4oc5ccccc5c4cc32)c1 ; N#Cc1ccc2c(c1)c1ccccc1n2-c1cccc(-n2c3ccccc3c3cc4sc5ccccc5c4cc32)c1]

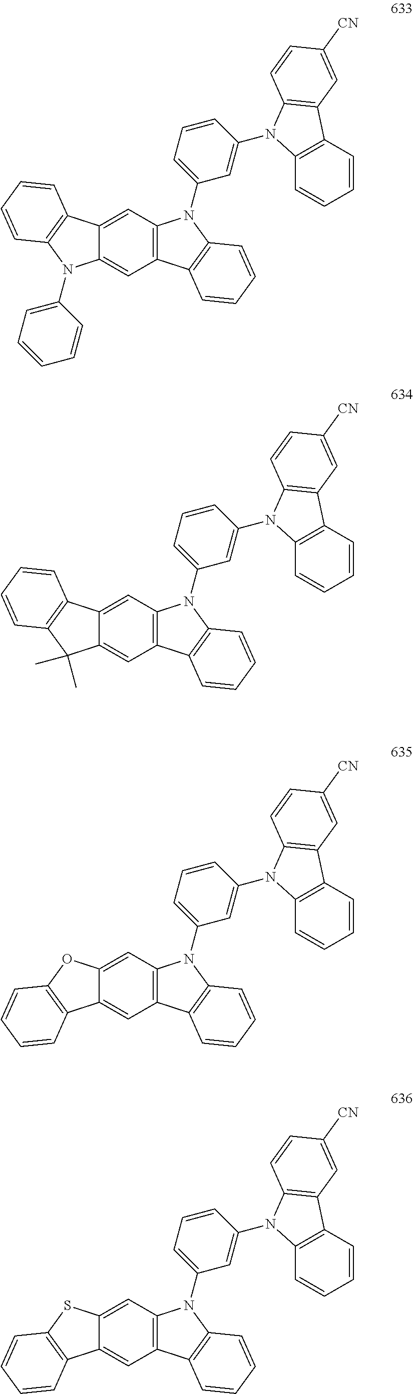 [SMILES: N#Cc1ccc2c(c1)c1ccccc1n2-c1cccc(-n2c3ccccc3c3cc4c(cc32)c2ccccc2n4-c2ccccc2)c1 ; CC1(C)c2ccccc2-c2cc3c(cc21)c1ccccc1n3-c1cccc(-n2c3ccccc3c3cc(C#N)ccc32)c1 ; N#Cc1ccc2c(c1)c1ccccc1n2-c1cccc(-n2c3ccccc3c3cc4c(cc32)oc2ccccc24)c1 ; N#Cc1ccc2c(c1)c1ccccc1n2-c1cccc(-n2c3ccccc3c3cc4c(cc32)sc2ccccc24)c1]

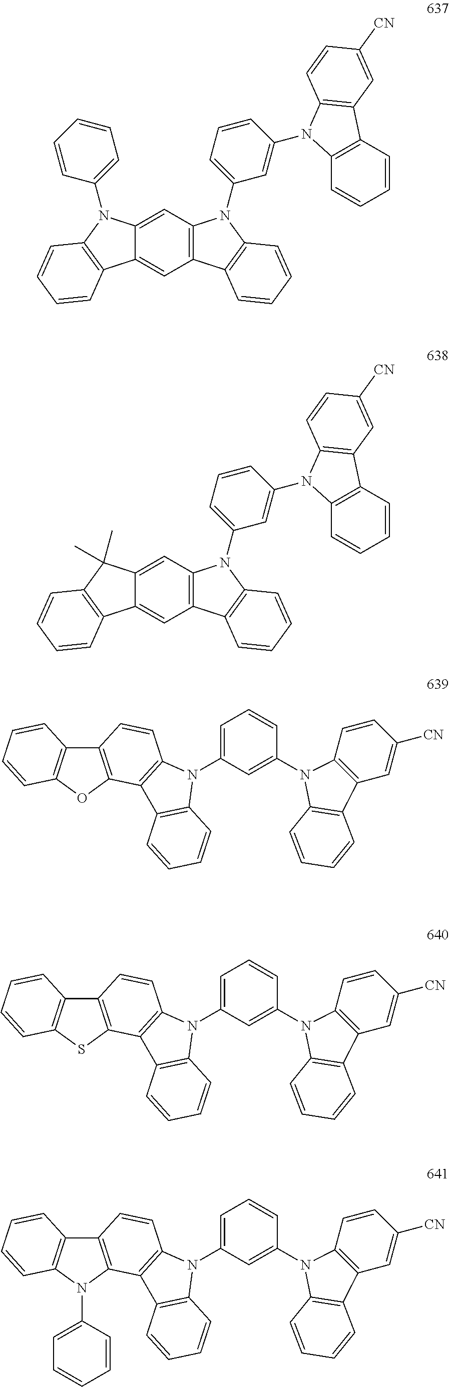 [SMILES: N#Cc1ccc2c(c1)c1ccccc1n2-c1cccc(-n2c3ccccc3c3cc4c5ccccc5n(-c5ccccc5)c4cc32)c1 ; CC1(C)c2ccccc2-c2cc3c4ccccc4n(-c4cccc(-n5c6ccccc6c6cc(C#N)ccc65)c4)c3cc21 ; N#Cc1ccc2c(c1)c1ccccc1n2-c1cccc(-n2c3ccccc3c3c4oc5ccccc5c4ccc32)c1 ; N#Cc1ccc2c(c1)c1ccccc1n2-c1cccc(-n2c3ccccc3c3c4sc5ccccc5c4ccc32)c1 ; N#Cc1ccc2c(c1)c1ccccc1n2-c1cccc(-n2c3ccccc3c3c2ccc2c4ccccc4n(-c4ccccc4)c23)c1]

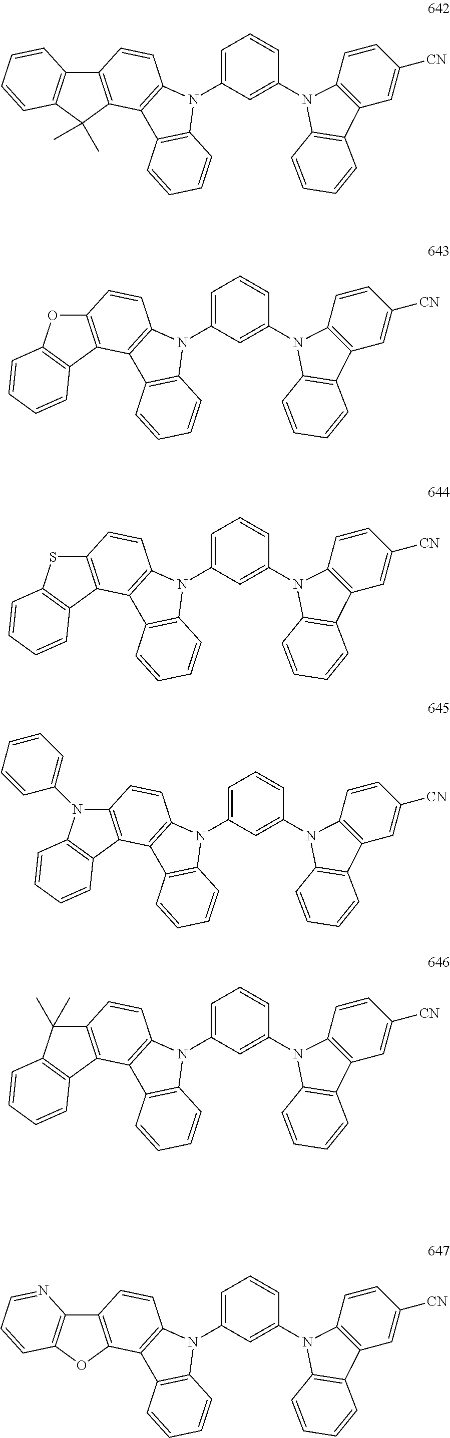 [SMILES: CC1(C)c2ccccc2-c2ccc3c(c21)c1ccccc1n3-c1cccc(-n2c3ccccc3c3cc(C#N)ccc32)c1 ; N#Cc1ccc2c(c1)c1ccccc1n2-c1cccc(-n2c3ccccc3c3c4c(ccc32)oc2ccccc24)c1 ; N#Cc1ccc2c(c1)c1ccccc1n2-c1cccc(-n2c3ccccc3c3c4c(ccc32)sc2ccccc24)c1 ; N#Cc1ccc2c(c1)c1ccccc1n2-c1cccc(-n2c3ccccc3c3c4c5ccccc5n(-c5ccccc5)c4ccc32)c1 ; CC1(C)c2ccccc2-c2c1ccc1c2c2ccccc2n1-c1cccc(-n2c3ccccc3c3cc(C#N)ccc32)c1 ; N#Cc1ccc2c(c1)c1ccccc1n2-c1cccc(-n2c3ccccc3c3c4oc5cccnc5c4ccc32)c1]

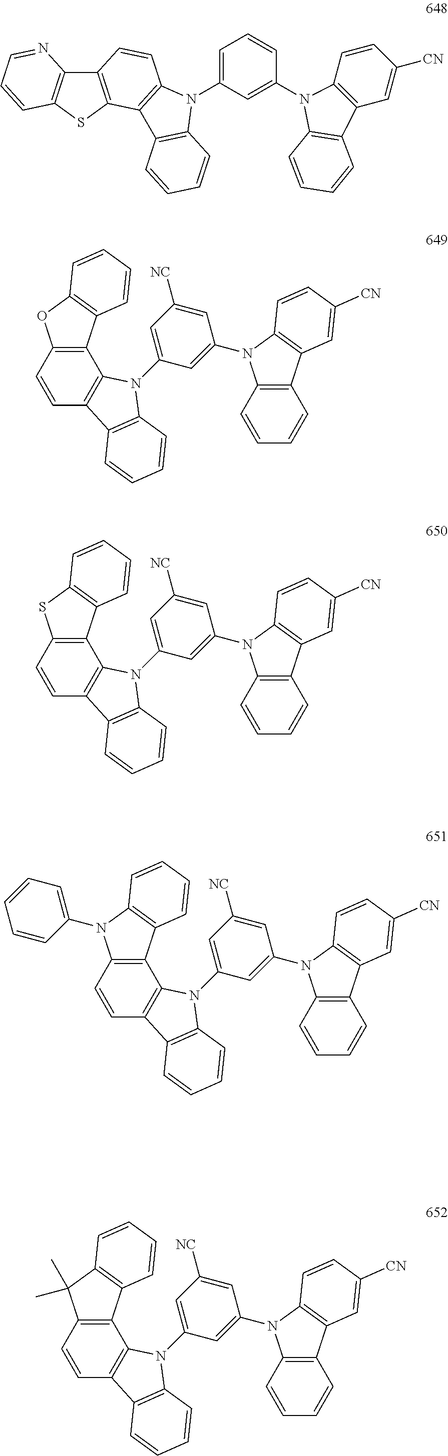 [SMILES: N#Cc1ccc2c(c1)c1ccccc1n2-c1cccc(-n2c3ccccc3c3c4sc5cccnc5c4ccc32)c1 ; N#Cc1cc(-n2c3ccccc3c3cc(C#N)ccc32)cc(-n2c3ccccc3c3ccc4oc5ccccc5c4c32)c1 ; N#Cc1cc(-n2c3ccccc3c3cc(C#N)ccc32)cc(-n2c3ccccc3c3ccc4sc5ccccc5c4c32)c1 ; N#Cc1cc(-n2c3ccccc3c3cc(C#N)ccc32)cc(-n2c3ccccc3c3ccc4c(c5ccccc5n4-c4ccccc4)c32)c1 ; CC1(C)c2ccccc2-c2c1ccc1c3ccccc3n(-c3cc(C#N)cc(-n4c5ccccc5c5cc(C#N)ccc54)c3)c21]

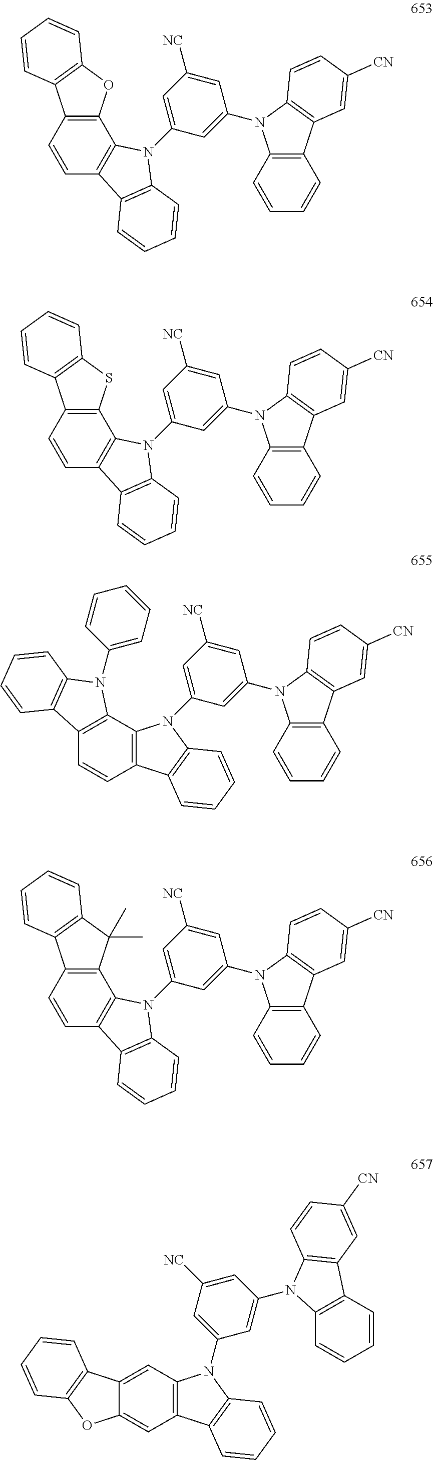

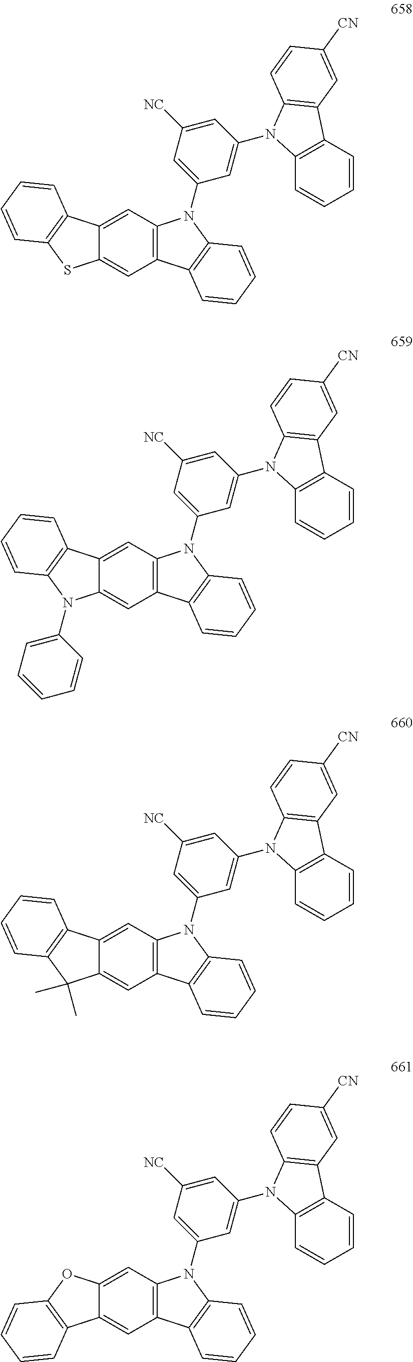 [SMILES: N#Cc1cc(-n2c3ccccc3c3cc(C#N)ccc32)cc(-n2c3ccccc3c3cc4sc5ccccc5c4cc32)c1 ; N#Cc1cc(-n2c3ccccc3c3cc(C#N)ccc32)cc(-n2c3ccccc3c3cc4c(cc32)c2ccccc2n4-c2ccccc2)c1 ; CC1(C)c2ccccc2-c2cc3c(cc21)c1ccccc1n3-c1cc(C#N)cc(-n2c3ccccc3c3cc(C#N)ccc32)c1 ; N#Cc1cc(-n2c3ccccc3c3cc(C#N)ccc32)cc(-n2c3ccccc3c3cc4c(cc32)oc2ccccc24)c1]

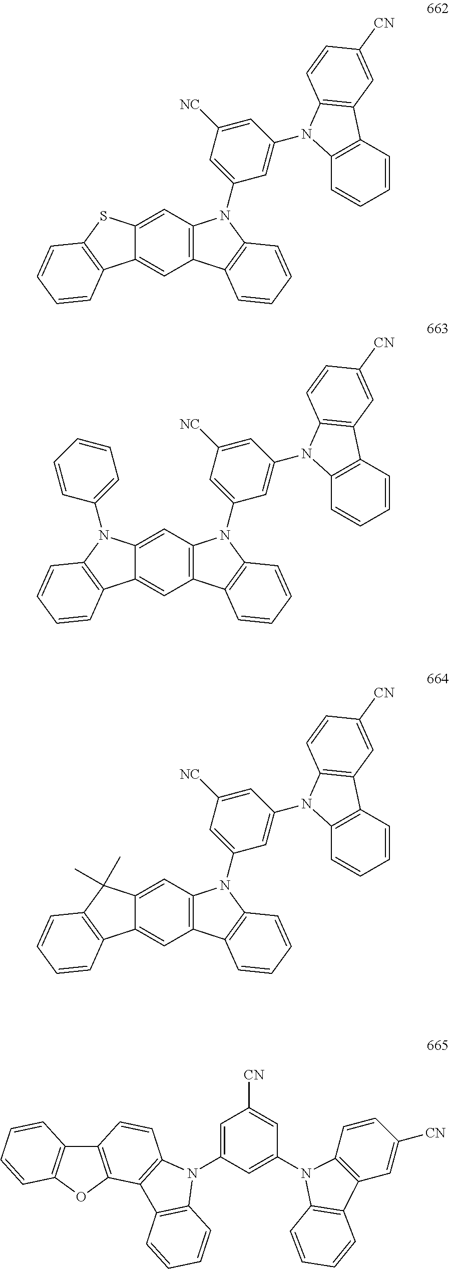 [SMILES: N#Cc1cc(-n2c3ccccc3c3cc(C#N)ccc32)cc(-n2c3ccccc3c3cc4c(cc32)sc2ccccc24)c1 ; N#Cc1cc(-n2c3ccccc3c3cc(C#N)ccc32)cc(-n2c3ccccc3c3cc4c5ccccc5n(-c5ccccc5)c4cc32)c1 ; CC1(C)c2ccccc2-c2cc3c4ccccc4n(-c4cc(C#N)cc(-n5c6ccccc6c6cc(C#N)ccc65)c4)c3cc21 ; N#Cc1cc(-n2c3ccccc3c3cc(C#N)ccc32)cc(-n2c3ccccc3c3c4oc5ccccc5c4ccc32)c1]

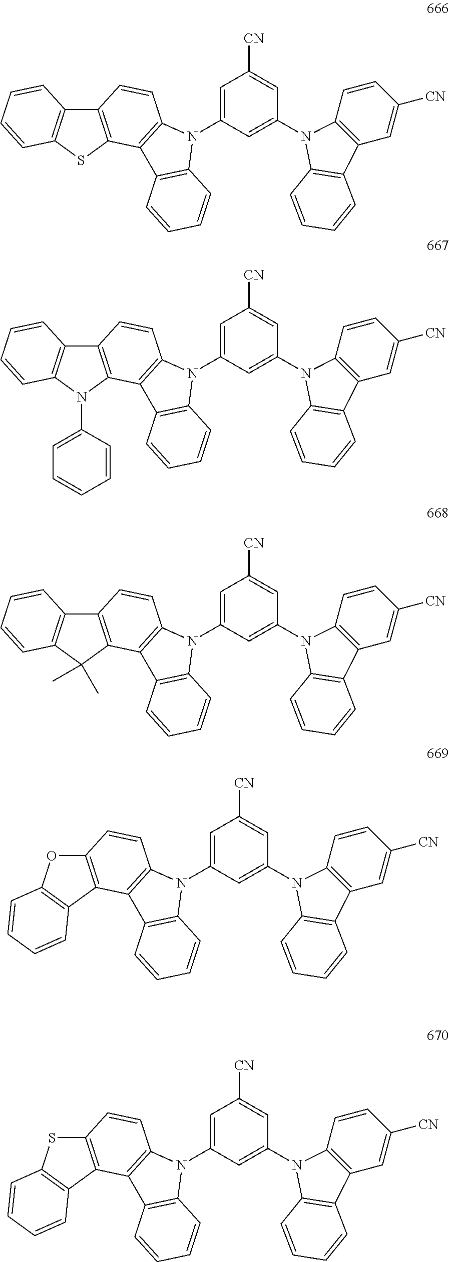 [SMILES: N#Cc1cc(-n2c3ccccc3c3cc(C#N)ccc32)cc(-n2c3ccccc3c3c4sc5ccccc5c4ccc32)c1 ; N#Cc1cc(-n2c3ccccc3c3cc(C#N)ccc32)cc(-n2c3ccccc3c3c2ccc2c4ccccc4n(-c4ccccc4)c23)c1 ; CC1(C)c2ccccc2-c2ccc3c(c21)c1ccccc1n3-c1cc(C#N)cc(-n2c3ccccc3c3cc(C#N)ccc32)c1 ; N#Cc1cc(-n2c3ccccc3c3cc(C#N)ccc32)cc(-n2c3ccccc3c3c4c(ccc32)oc2ccccc24)c1 ; N#Cc1cc(-n2c3ccccc3c3cc(C#N)ccc32)cc(-n2c3ccccc3c3c4c(ccc32)sc2ccccc24)c1]

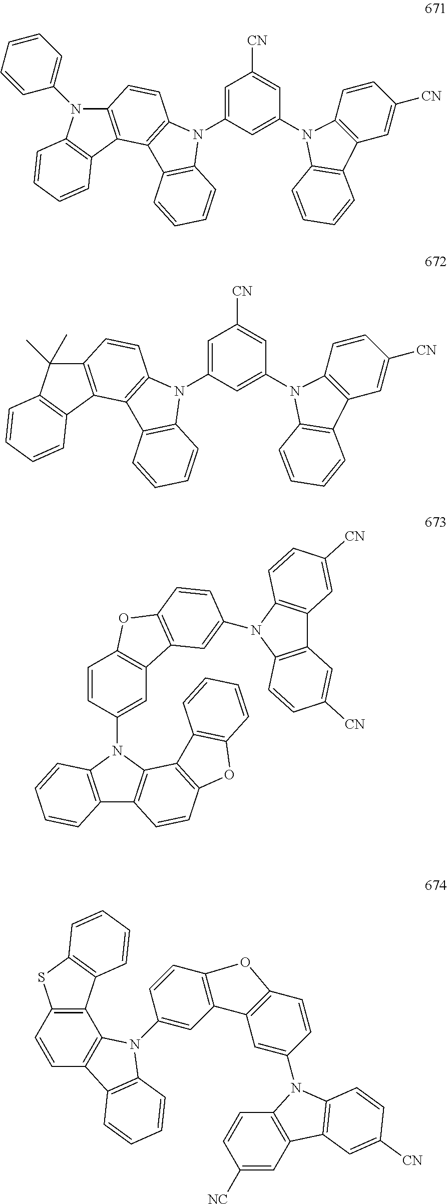 [SMILES: N#Cc1cc(-n2c3ccccc3c3cc(C#N)ccc32)cc(-n2c3ccccc3c3c4c5ccccc5n(-c5ccccc5)c4ccc32)c1 ; CC1(C)c2ccccc2-c2c1ccc1c2c2ccccc2n1-c1cc(C#N)cc(-n2c3ccccc3c3cc(C#N)ccc32)c1 ; N#Cc1ccc2c(c1)c1cc(C#N)ccc1n2-c1ccc2oc3ccc(-n4c5ccccc5c5ccc6oc7ccccc7c6c54)cc3c2c1 ; N#Cc1ccc2c(c1)c1cc(C#N)ccc1n2-c1ccc2oc3ccc(-n4c5ccccc5c5ccc6sc7ccccc7c6c54)cc3c2c1]

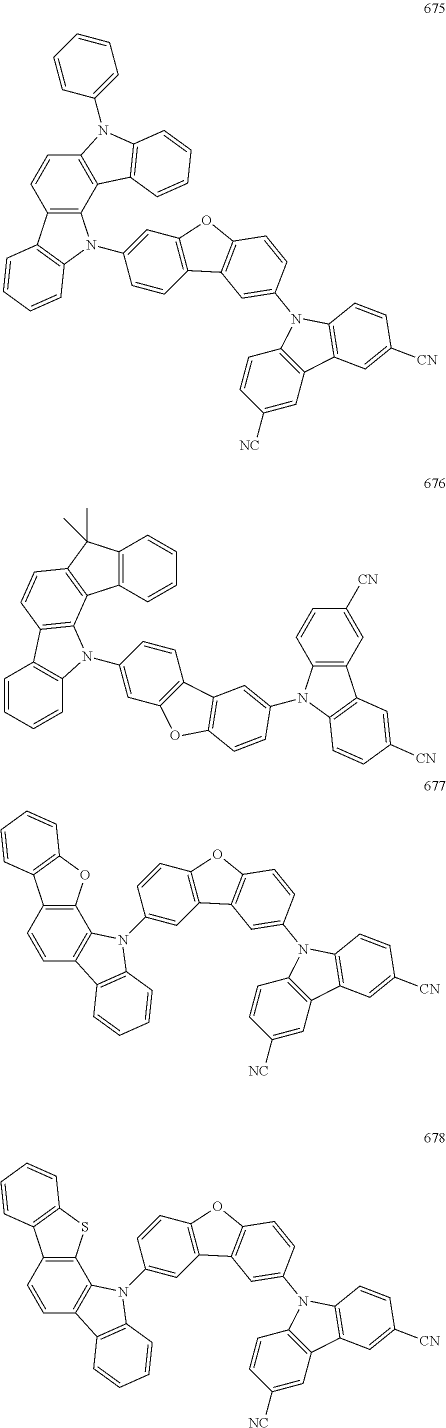 [SMILES: N#Cc1ccc2c(c1)c1cc(C#N)ccc1n2-c1ccc2oc3cc(-n4c5ccccc5c5ccc6c(c7ccccc7n6-c6ccccc6)c54)ccc3c2c1 ; CC1(C)c2ccccc2-c2c1ccc1c3ccccc3n(-c3ccc4c(c3)oc3ccc(-n5c6ccc(C#N)cc6c6cc(C#N)ccc65)cc34)c21 ; N#Cc1ccc2c(c1)c1cc(C#N)ccc1n2-c1ccc2oc3ccc(-n4c5ccccc5c5ccc6c7ccccc7oc6c54)cc3c2c1 ; N#Cc1ccc2c(c1)c1cc(C#N)ccc1n2-c1ccc2oc3ccc(-n4c5ccccc5c5ccc6c7ccccc7sc6c54)cc3c2c1]

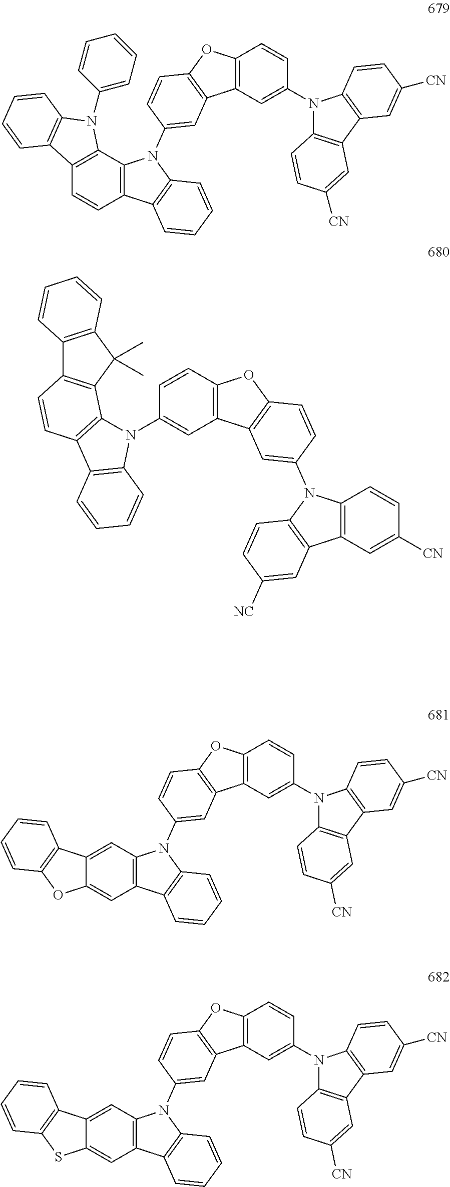 [SMILES: N#Cc1ccc2c(c1)c1cc(C#N)ccc1n2-c1ccc2oc3ccc(-n4c5ccccc5c5ccc6c7ccccc7n(-c7ccccc7)c6c54)cc3c2c1 ; CC1(C)c2ccccc2-c2ccc3c4ccccc4n(-c4ccc5oc6ccc(-n7c8ccc(C#N)cc8c8cc(C#N)ccc87)cc6c5c4)c3c21 ; N#Cc1ccc2c(c1)c1cc(C#N)ccc1n2-c1ccc2oc3ccc(-n4c5ccccc5c5cc6oc7ccccc7c6cc54)cc3c2c1 ; N#Cc1ccc2c(c1)c1cc(C#N)ccc1n2-c1ccc2oc3ccc(-n4c5ccccc5c5cc6sc7ccccc7c6cc54)cc3c2c1]

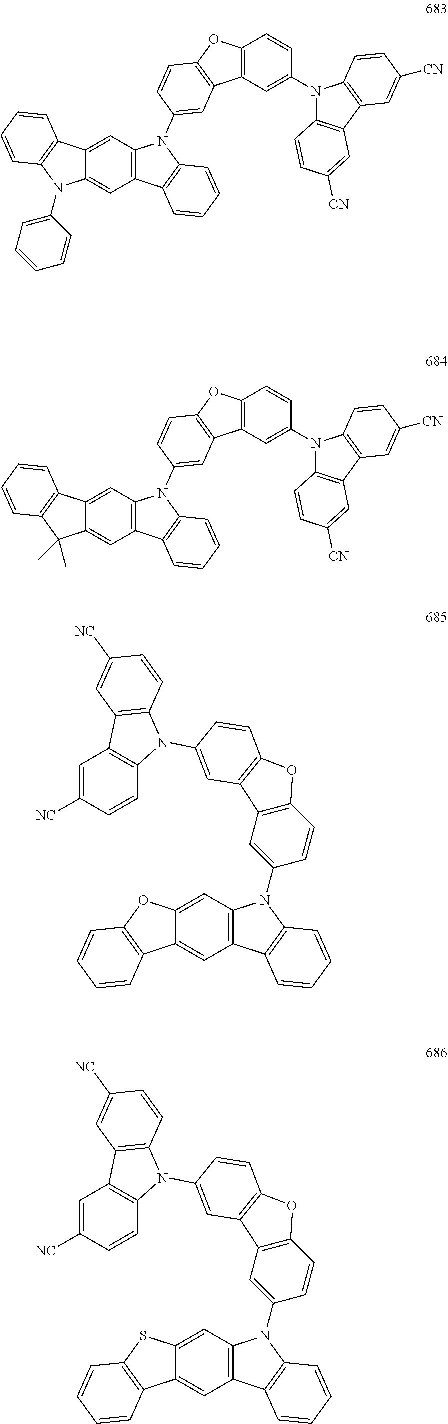 [SMILES: N#Cc1ccc2c(c1)c1cc(C#N)ccc1n2-c1ccc2oc3ccc(-n4c5ccccc5c5cc6c(cc54)c4ccccc4n6-c4ccccc4)cc3c2c1 ; CC1(C)c2ccccc2-c2cc3c(cc21)c1ccccc1n3-c1ccc2oc3ccc(-n4c5ccc(C#N)cc5c5cc(C#N)ccc54)cc3c2c1 ; N#Cc1ccc2c(c1)c1cc(C#N)ccc1n2-c1ccc2oc3ccc(-n4c5ccccc5c5cc6c(cc54)oc4ccccc46)cc3c2c1 ; N#Cc1ccc2c(c1)c1cc(C#N)ccc1n2-c1ccc2oc3ccc(-n4c5ccccc5c5cc6c(cc54)sc4ccccc46)cc3c2c1]

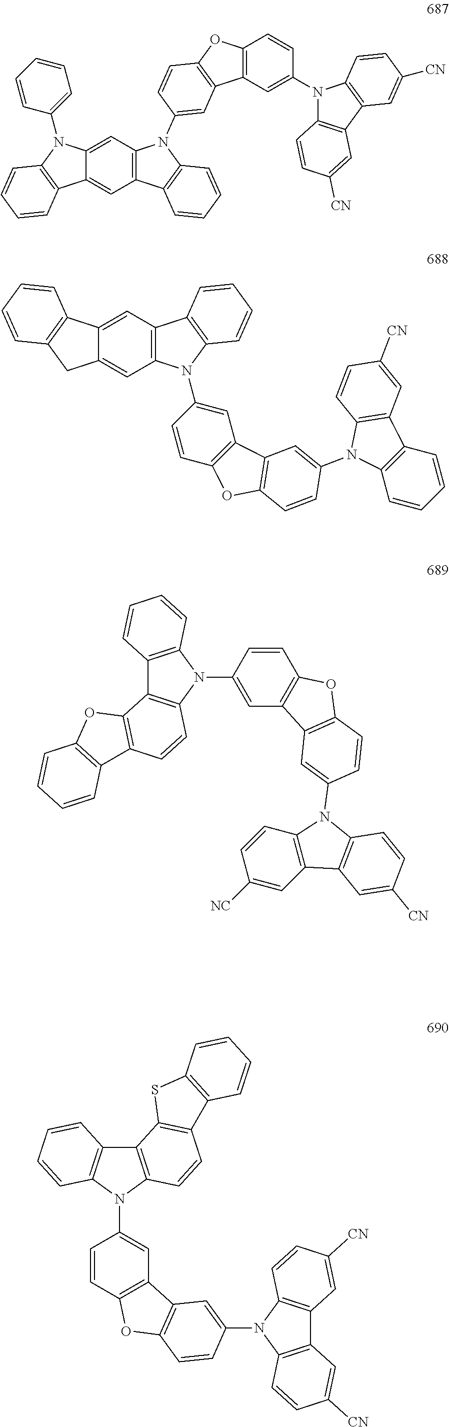 [SMILES: N#Cc1ccc2c(c1)c1cc(C#N)ccc1n2-c1ccc2oc3ccc(-n4c5ccccc5c5cc6c7ccccc7n(-c7ccccc7)c6cc54)cc3c2c1 ; N#Cc1ccc2c(c1)c1ccccc1n2-c1ccc2oc3ccc(-n4c5ccccc5c5cc6c(cc54)Cc4ccccc4-6)cc3c2c1 ; N#Cc1ccc2c(c1)c1cc(C#N)ccc1n2-c1ccc2oc3ccc(-n4c5ccccc5c5c6oc7ccccc7c6ccc54)cc3c2c1 ; N#Cc1ccc2c(c1)c1cc(C#N)ccc1n2-c1ccc2oc3ccc(-n4c5ccccc5c5c6sc7ccccc7c6ccc54)cc3c2c1]

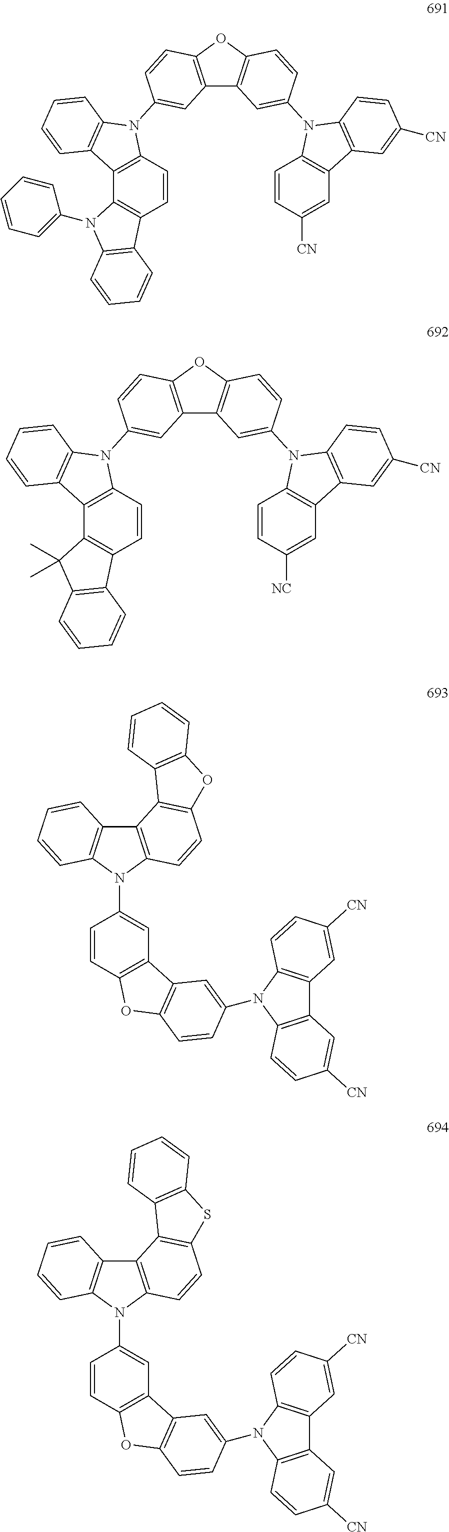 [SMILES: N#Cc1ccc2c(c1)c1cc(C#N)ccc1n2-c1ccc2oc3ccc(-n4c5ccccc5c5c4ccc4c6ccccc6n(-c6ccccc6)c45)cc3c2c1 ; CC1(C)c2ccccc2-c2ccc3c(c21)c1ccccc1n3-c1ccc2oc3ccc(-n4c5ccc(C#N)cc5c5cc(C#N)ccc54)cc3c2c1 ; N#Cc1ccc2c(c1)c1cc(C#N)ccc1n2-c1ccc2oc3ccc(-n4c5ccccc5c5c6c(ccc54)oc4ccccc46)cc3c2c1 ; N#Cc1ccc2c(c1)c1cc(C#N)ccc1n2-c1ccc2oc3ccc(-n4c5ccccc5c5c6c(ccc54)sc4ccccc46)cc3c2c1]

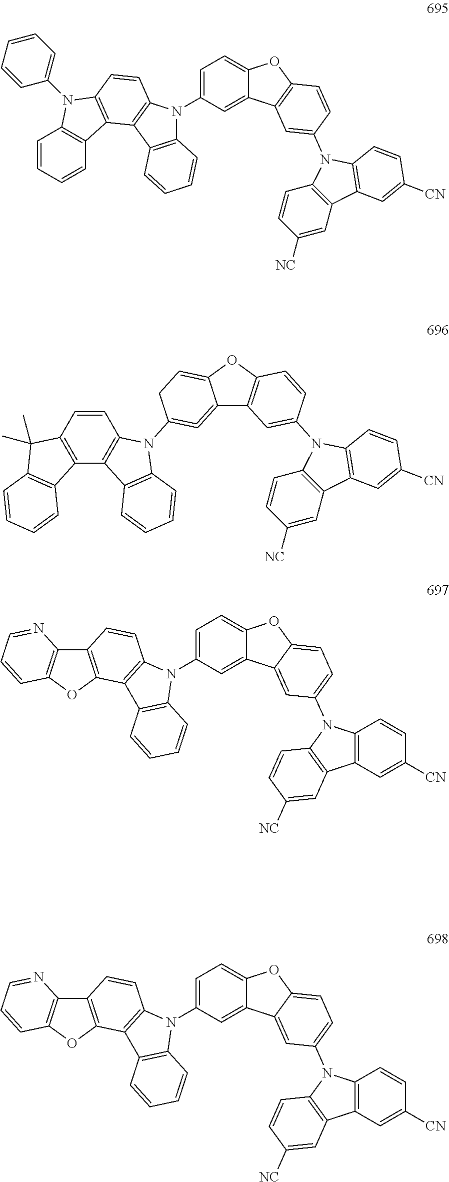 [SMILES: N#Cc1ccc2c(c1)c1cc(C#N)ccc1n2-c1ccc2oc3ccc(-n4c5ccccc5c5c6c7ccccc7n(-c7ccccc7)c6ccc54)cc3c2c1 ; CC1(C)c2ccccc2-c2c1ccc1c2c2ccccc2n1C1=C=c2c(oc3ccc(-n4c5ccc(C#N)cc5c5cc(C#N)ccc54)cc23)=CC1 ; N#Cc1ccc2c(c1)c1cc(C#N)ccc1n2-c1ccc2oc3ccc(-n4c5ccccc5c5c6oc7cccnc7c6ccc54)cc3c2c1 ; N#Cc1ccc2c(c1)c1cc(C#N)ccc1n2-c1ccc2oc3ccc(-n4c5ccccc5c5c6oc7cccnc7c6ccc54)cc3c2c1]

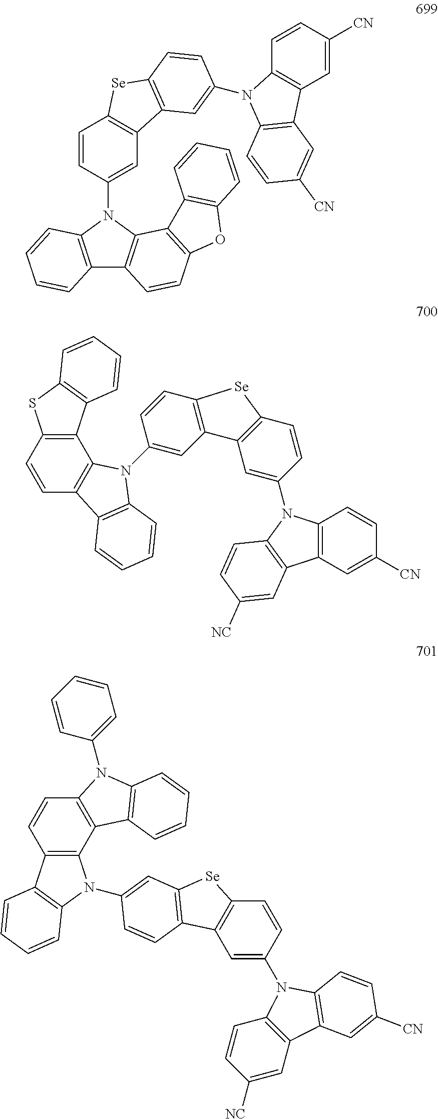 [SMILES: N#Cc1ccc2c(c1)c1cc(C#N)ccc1n2-c1ccc2[se]c3ccc(-n4c5ccccc5c5ccc6oc7ccccc7c6c54)cc3c2c1 ; N#Cc1ccc2c(c1)c1cc(C#N)ccc1n2-c1ccc2[se]c3ccc(-n4c5ccccc5c5ccc6sc7ccccc7c6c54)cc3c2c1 ; N#Cc1ccc2c(c1)c1cc(C#N)ccc1n2-c1ccc2[se]c3cc(-n4c5ccccc5c5ccc6c(c7ccccc7n6-c6ccccc6)c54)ccc3c2c1]

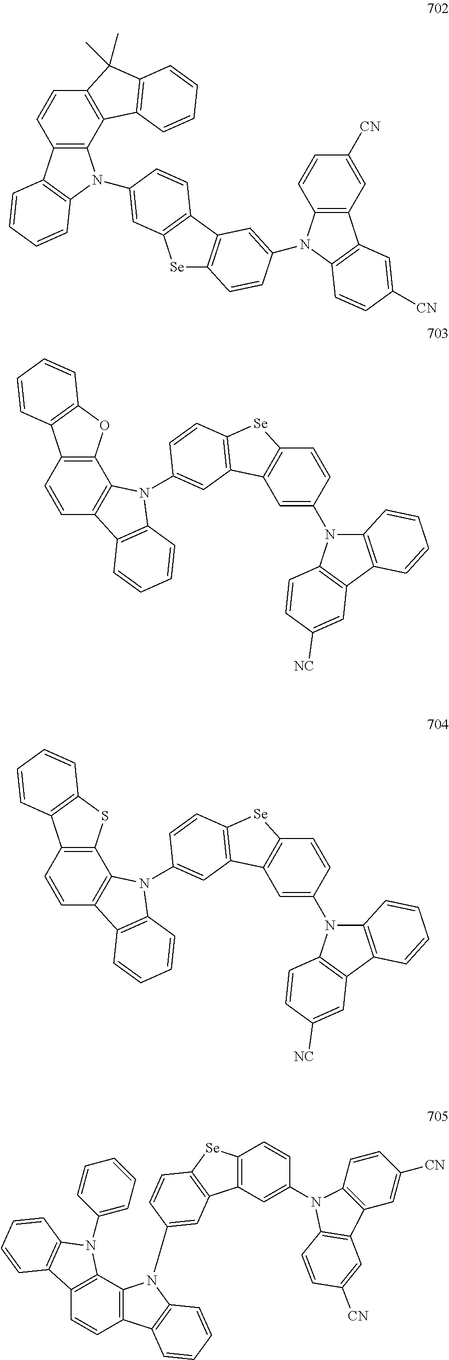 [SMILES: CC1(C)c2ccccc2-c2c1ccc1c3ccccc3n(-c3ccc4c(c3)[se]c3ccc(-n5c6ccc(C#N)cc6c6cc(C#N)ccc65)cc34)c21 ; N#Cc1ccc2c(c1)c1ccccc1n2-c1ccc2[se]c3ccc(-n4c5ccccc5c5ccc6c7ccccc7oc6c54)cc3c2c1 ; N#Cc1ccc2c(c1)c1ccccc1n2-c1ccc2[se]c3ccc(-n4c5ccccc5c5ccc6c7ccccc7sc6c54)cc3c2c1 ; N#Cc1ccc2c(c1)c1cc(C#N)ccc1n2-c1ccc2[se]c3ccc(-n4c5ccccc5c5ccc6c7ccccc7n(-c7ccccc7)c6c54)cc3c2c1]

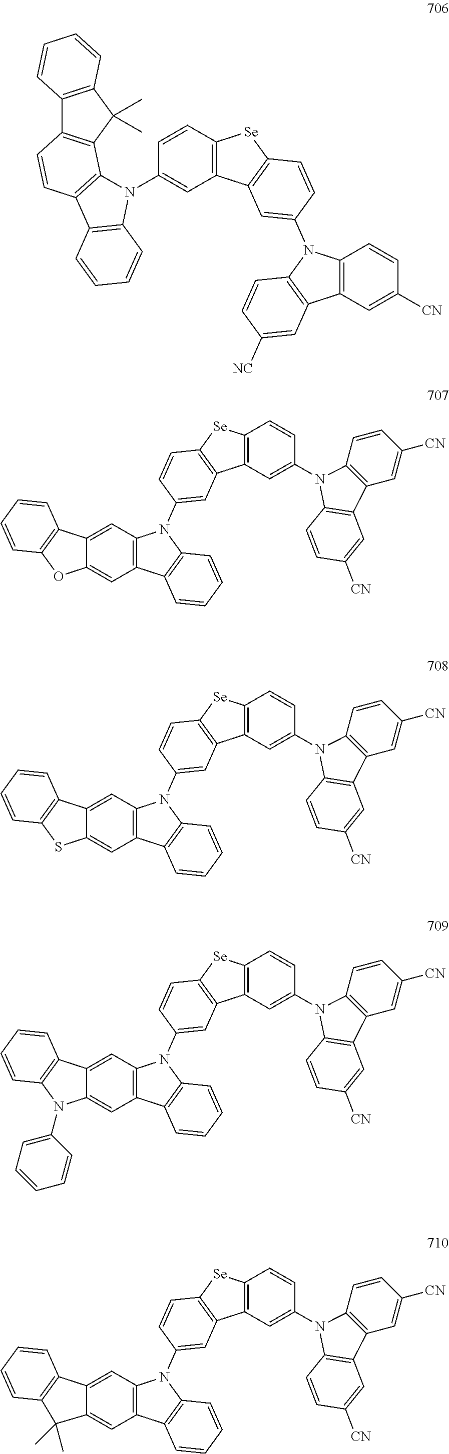 [SMILES: CC1(C)c2ccccc2-c2ccc3c4ccccc4n(-c4ccc5[se]c6ccc(-n7c8ccc(C#N)cc8c8cc(C#N)ccc87)cc6c5c4)c3c21 ; N#Cc1ccc2c(c1)c1cc(C#N)ccc1n2-c1ccc2[se]c3ccc(-n4c5ccccc5c5cc6oc7ccccc7c6cc54)cc3c2c1 ; N#Cc1ccc2c(c1)c1cc(C#N)ccc1n2-c1ccc2[se]c3ccc(-n4c5ccccc5c5cc6sc7ccccc7c6cc54)cc3c2c1 ; N#Cc1ccc2c(c1)c1cc(C#N)ccc1n2-c1ccc2[se]c3ccc(-n4c5ccccc5c5cc6c(cc54)c4ccccc4n6-c4ccccc4)cc3c2c1 ; CC1(C)c2ccccc2-c2cc3c(cc21)c1ccccc1n3-c1ccc2[se]c3ccc(-n4c5ccc(C#N)cc5c5cc(C#N)ccc54)cc3c2c1]

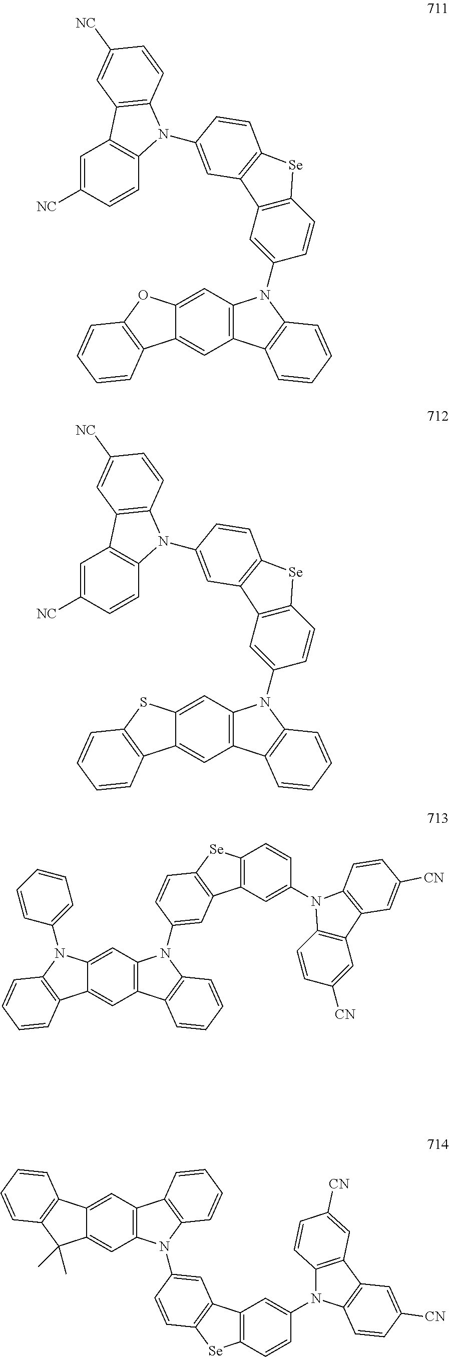 [SMILES: N#Cc1ccc2c(c1)c1cc(C#N)ccc1n2-c1ccc2[se]c3ccc(-n4c5ccccc5c5cc6c(cc54)oc4ccccc46)cc3c2c1 ; N#Cc1ccc2c(c1)c1cc(C#N)ccc1n2-c1ccc2[se]c3ccc(-n4c5ccccc5c5cc6c(cc54)sc4ccccc46)cc3c2c1 ; N#Cc1ccc2c(c1)c1cc(C#N)ccc1n2-c1ccc2[se]c3ccc(-n4c5ccccc5c5cc6c7ccccc7n(-c7ccccc7)c6cc54)cc3c2c1 ; CC1(C)c2ccccc2-c2cc3c4ccccc4n(-c4ccc5[se]c6ccc(-n7c8ccc(C#N)cc8c8cc(C#N)ccc87)cc6c5c4)c3cc21]

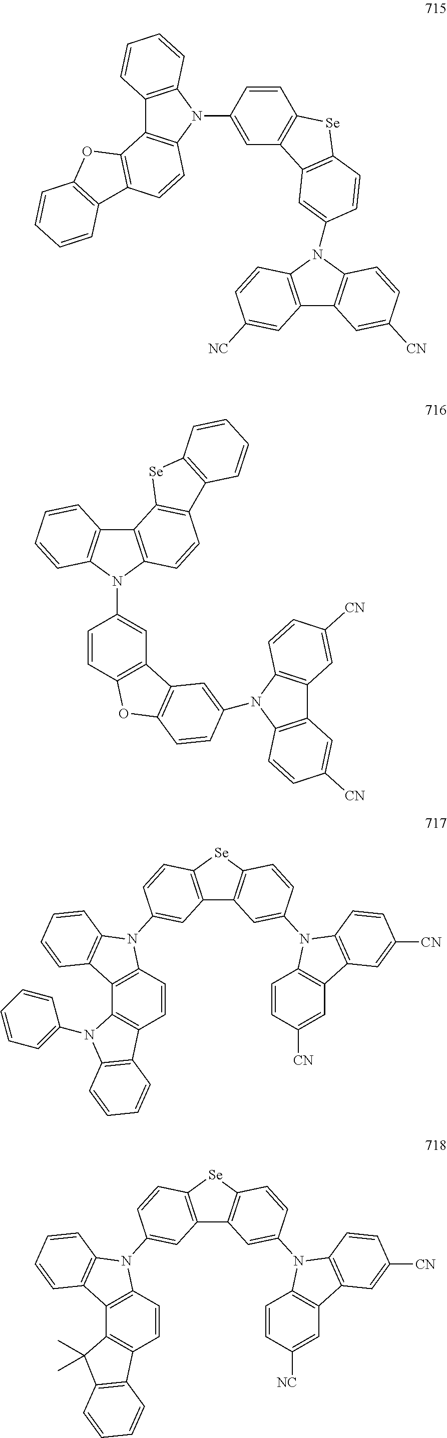 [SMILES: N#Cc1ccc2c(c1)c1cc(C#N)ccc1n2-c1ccc2[se]c3ccc(-n4c5ccccc5c5c6oc7ccccc7c6ccc54)cc3c2c1 ; N#Cc1ccc2c(c1)c1cc(C#N)ccc1n2-c1ccc2oc3ccc(-n4c5ccccc5c5c6[se]c7ccccc7c6ccc54)cc3c2c1 ; N#Cc1ccc2c(c1)c1cc(C#N)ccc1n2-c1ccc2[se]c3ccc(-n4c5ccccc5c5c4ccc4c6ccccc6n(-c6ccccc6)c45)cc3c2c1 ; CC1(C)c2ccccc2-c2ccc3c(c21)c1ccccc1n3-c1ccc2[se]c3ccc(-n4c5ccc(C#N)cc5c5cc(C#N)ccc54)cc3c2c1]

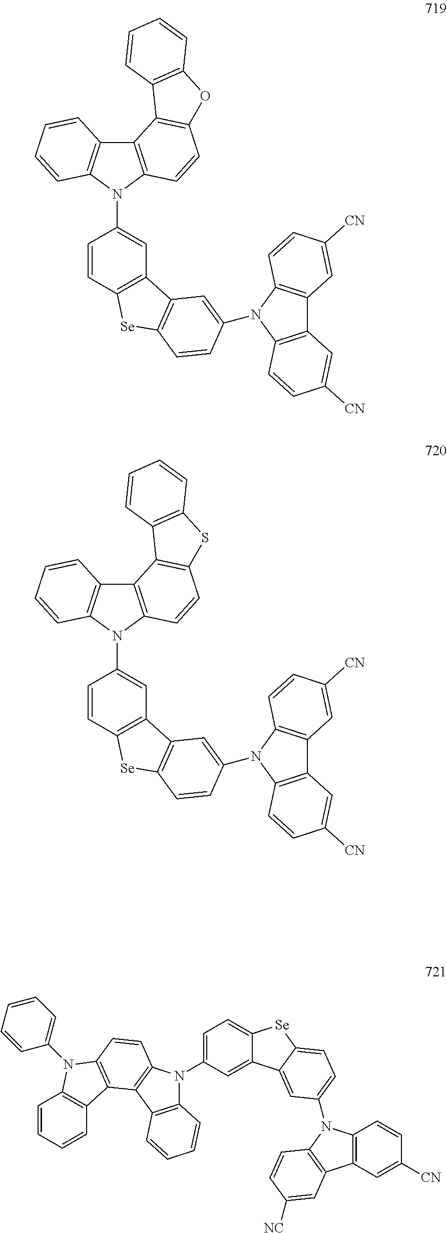 [SMILES: N#Cc1ccc2c(c1)c1cc(C#N)ccc1n2-c1ccc2[se]c3ccc(-n4c5ccccc5c5c6c(ccc54)oc4ccccc46)cc3c2c1 ; N#Cc1ccc2c(c1)c1cc(C#N)ccc1n2-c1ccc2[se]c3ccc(-n4c5ccccc5c5c6c(ccc54)sc4ccccc46)cc3c2c1 ; N#Cc1ccc2c(c1)c1cc(C#N)ccc1n2-c1ccc2[se]c3ccc(-n4c5ccccc5c5c6c7ccccc7n(-c7ccccc7)c6ccc54)cc3c2c1]

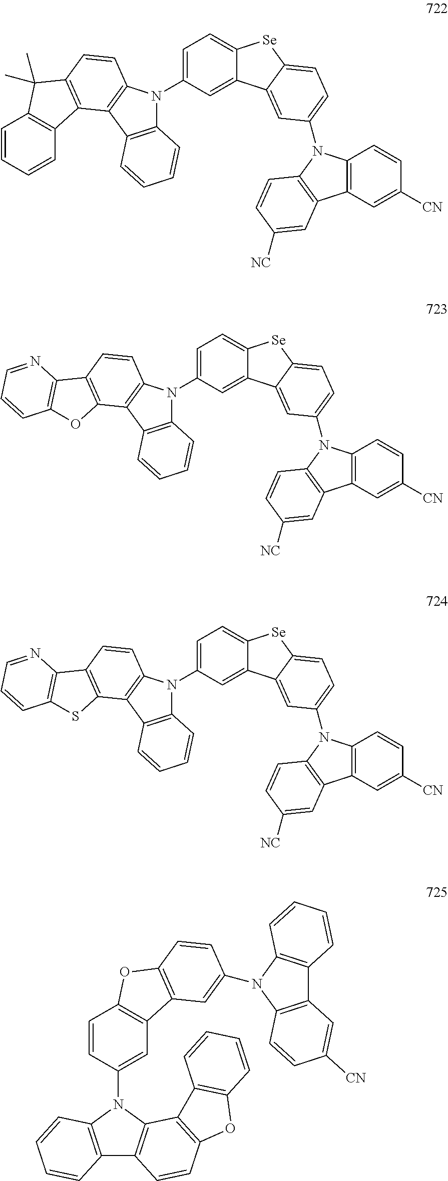 [SMILES: CC1(C)c2ccccc2-c2c1ccc1c2c2ccccc2n1-c1ccc2[se]c3ccc(-n4c5ccc(C#N)cc5c5cc(C#N)ccc54)cc3c2c1 ; N#Cc1ccc2c(c1)c1cc(C#N)ccc1n2-c1ccc2[se]c3ccc(-n4c5ccccc5c5c6oc7cccnc7c6ccc54)cc3c2c1 ; N#Cc1ccc2c(c1)c1cc(C#N)ccc1n2-c1ccc2[se]c3ccc(-n4c5ccccc5c5c6sc7cccnc7c6ccc54)cc3c2c1 ; N#Cc1ccc2c(c1)c1ccccc1n2-c1ccc2oc3ccc(-n4c5ccccc5c5ccc6oc7ccccc7c6c54)cc3c2c1]

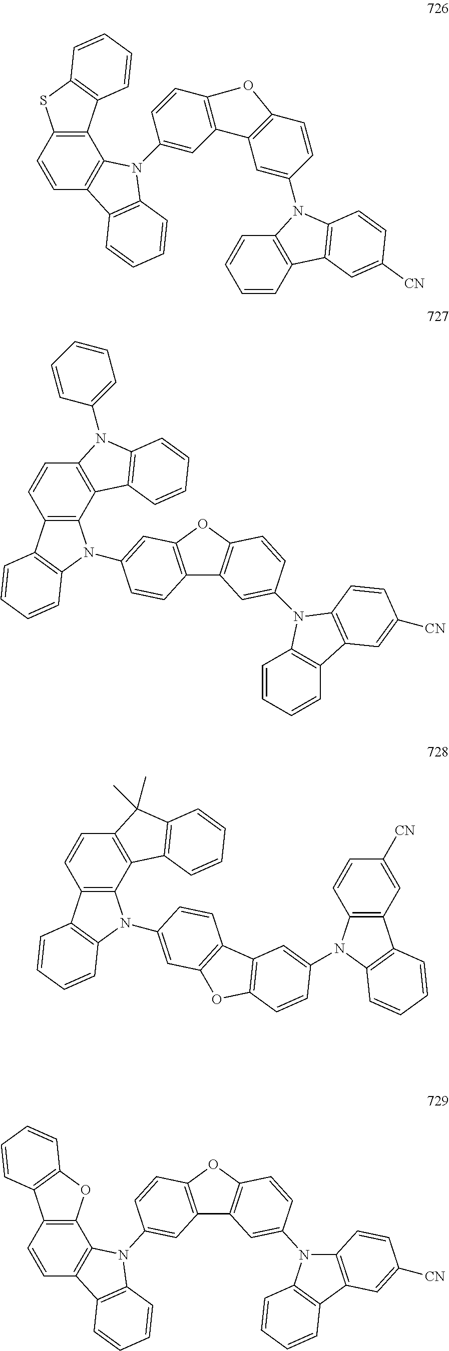 [SMILES: N#Cc1ccc2c(c1)c1ccccc1n2-c1ccc2oc3ccc(-n4c5ccccc5c5ccc6sc7ccccc7c6c54)cc3c2c1 ; N#Cc1ccc2c(c1)c1ccccc1n2-c1ccc2oc3cc(-n4c5ccccc5c5ccc6c(c7ccccc7n6-c6ccccc6)c54)ccc3c2c1 ; CC1(C)c2ccccc2-c2c1ccc1c3ccccc3n(-c3ccc4c(c3)oc3ccc(-n5c6ccccc6c6cc(C#N)ccc65)cc34)c21 ; N#Cc1ccc2c(c1)c1ccccc1n2-c1ccc2oc3ccc(-n4c5ccccc5c5ccc6c7ccccc7oc6c54)cc3c2c1]

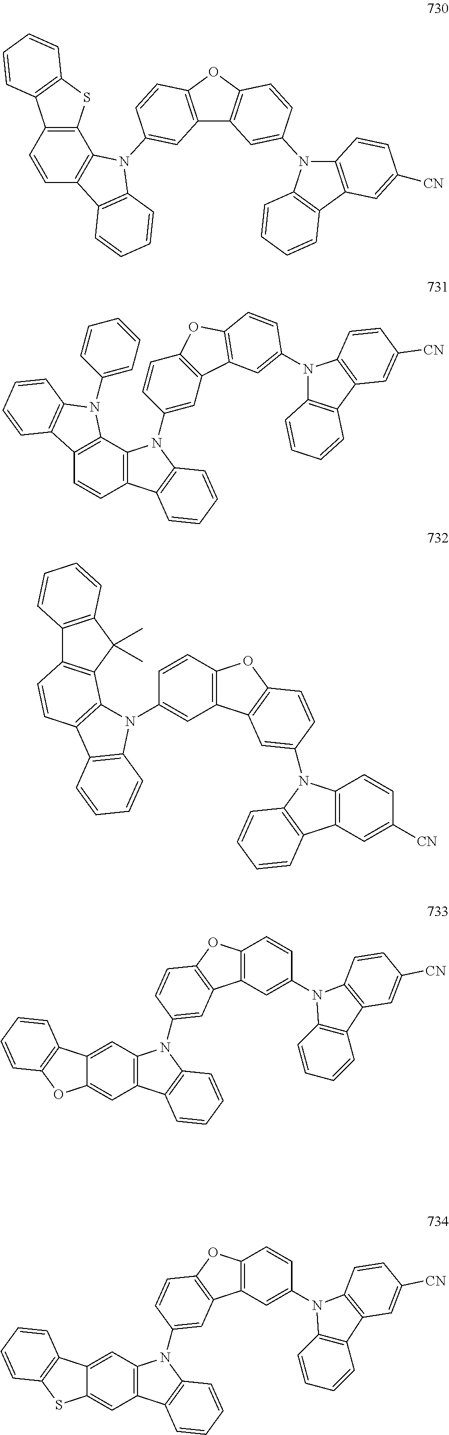 [SMILES: N#Cc1ccc2c(c1)c1ccccc1n2-c1ccc2oc3ccc(-n4c5ccccc5c5ccc6c7ccccc7sc6c54)cc3c2c1 ; N#Cc1ccc2c(c1)c1ccccc1n2-c1ccc2oc3ccc(-n4c5ccccc5c5ccc6c7ccccc7n(-c7ccccc7)c6c54)cc3c2c1 ; CC1(C)c2ccccc2-c2ccc3c4ccccc4n(-c4ccc5oc6ccc(-n7c8ccccc8c8cc(C#N)ccc87)cc6c5c4)c3c21 ; N#Cc1ccc2c(c1)c1ccccc1n2-c1ccc2oc3ccc(-n4c5ccccc5c5cc6oc7ccccc7c6cc54)cc3c2c1 ; N#Cc1ccc2c(c1)c1ccccc1n2-c1ccc2oc3ccc(-n4c5ccccc5c5cc6sc7ccccc7c6cc54)cc3c2c1]

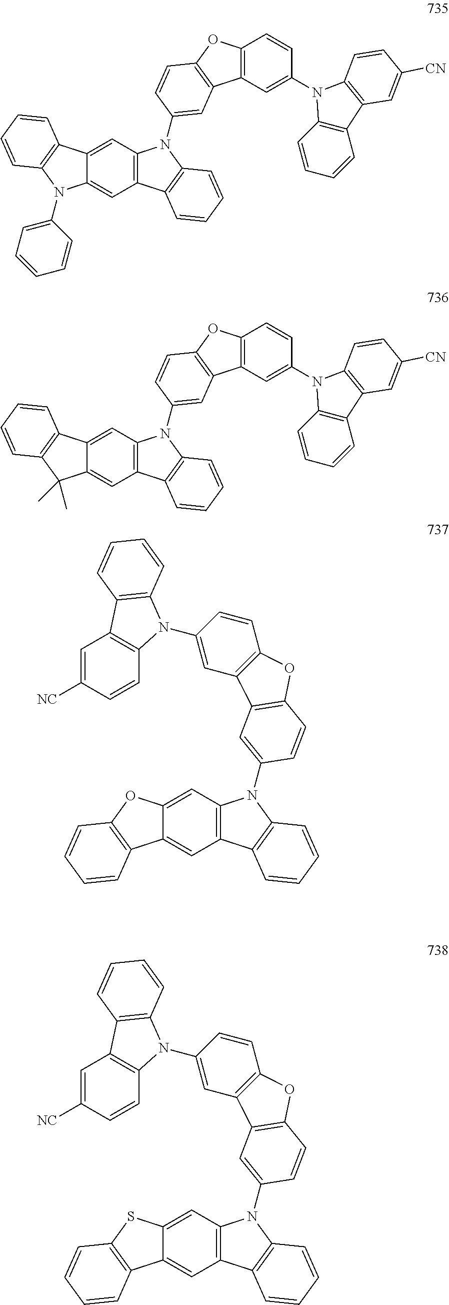 [SMILES: N#Cc1ccc2c(c1)c1ccccc1n2-c1ccc2oc3ccc(-n4c5ccccc5c5cc6c(cc54)c4ccccc4n6-c4ccccc4)cc3c2c1 ; CC1(C)c2ccccc2-c2cc3c(cc21)c1ccccc1n3-c1ccc2oc3ccc(-n4c5ccccc5c5cc(C#N)ccc54)cc3c2c1 ; N#Cc1ccc2c(c1)c1ccccc1n2-c1ccc2oc3ccc(-n4c5ccccc5c5cc6c(cc54)oc4ccccc46)cc3c2c1 ; N#Cc1ccc2c(c1)c1ccccc1n2-c1ccc2oc3ccc(-n4c5ccccc5c5cc6c(cc54)sc4ccccc46)cc3c2c1]

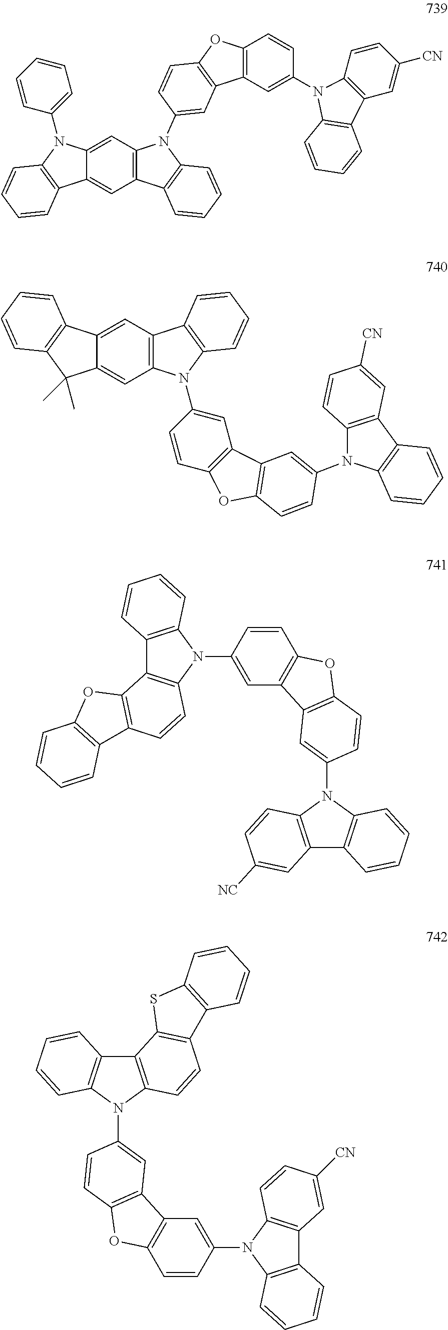 [SMILES: N#Cc1ccc2c(c1)c1ccccc1n2-c1ccc2oc3ccc(-n4c5ccccc5c5cc6c7ccccc7n(-c7ccccc7)c6cc54)cc3c2c1 ; CC1(C)c2ccccc2-c2cc3c4ccccc4n(-c4ccc5oc6ccc(-n7c8ccccc8c8cc(C#N)ccc87)cc6c5c4)c3cc21 ; N#Cc1ccc2c(c1)c1ccccc1n2-c1ccc2oc3ccc(-n4c5ccccc5c5c6oc7ccccc7c6ccc54)cc3c2c1 ; N#Cc1ccc2c(c1)c1ccccc1n2-c1ccc2oc3ccc(-n4c5ccccc5c5c6sc7ccccc7c6ccc54)cc3c2c1]

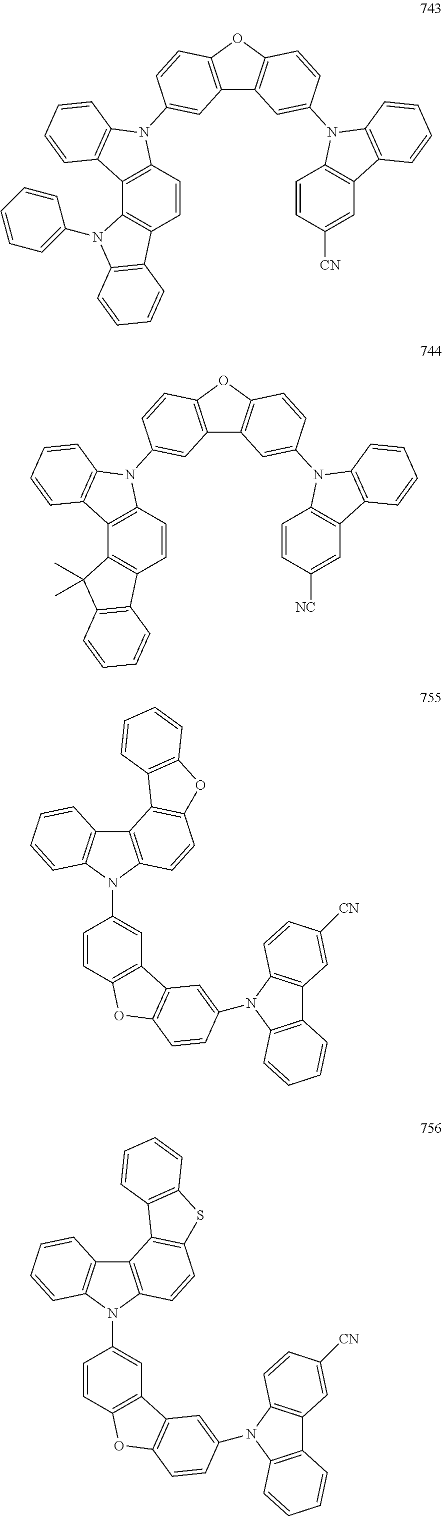 [SMILES: N#Cc1ccc2c(c1)c1ccccc1n2-c1ccc2oc3ccc(-n4c5ccccc5c5c4ccc4c6ccccc6n(-c6ccccc6)c45)cc3c2c1 ; CC1(C)c2ccccc2-c2ccc3c(c21)c1ccccc1n3-c1ccc2oc3ccc(-n4c5ccccc5c5cc(C#N)ccc54)cc3c2c1 ; N#Cc1ccc2c(c1)c1ccccc1n2-c1ccc2oc3ccc(-n4c5ccccc5c5c6c(ccc54)oc4ccccc46)cc3c2c1 ; N#Cc1ccc2c(c1)c1ccccc1n2-c1ccc2oc3ccc(-n4c5ccccc5c5c6c(ccc54)sc4ccccc46)cc3c2c1]

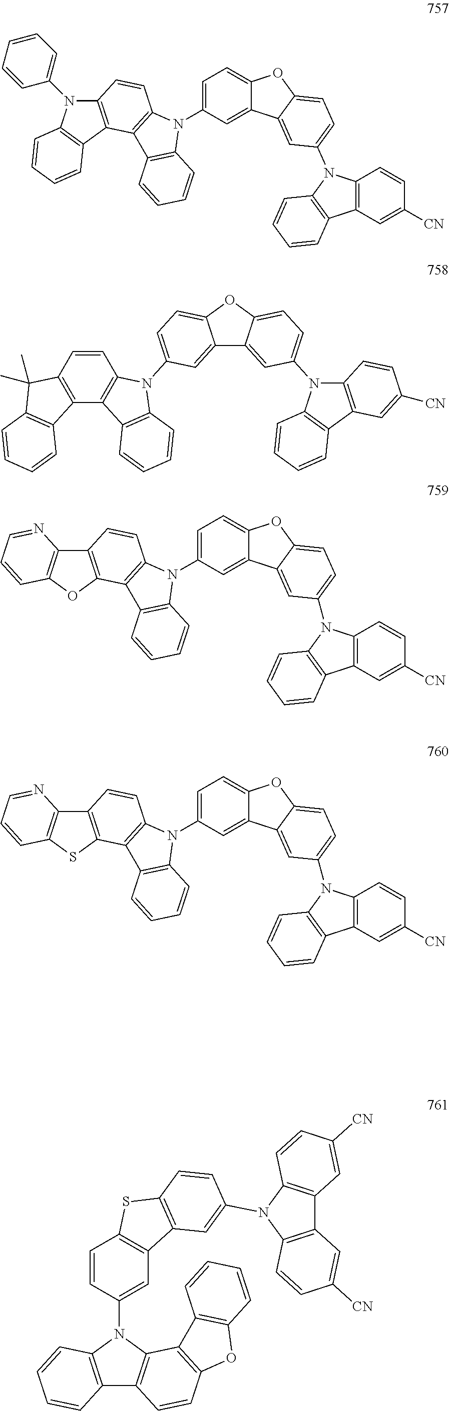 [SMILES: N#Cc1ccc2c(c1)c1ccccc1n2-c1ccc2oc3ccc(-n4c5ccccc5c5c6c7ccccc7n(-c7ccccc7)c6ccc54)cc3c2c1 ; CC1(C)c2ccccc2-c2c1ccc1c2c2ccccc2n1-c1ccc2oc3ccc(-n4c5ccccc5c5cc(C#N)ccc54)cc3c2c1 ; N#Cc1ccc2c(c1)c1ccccc1n2-c1ccc2oc3ccc(-n4c5ccccc5c5c6oc7cccnc7c6ccc54)cc3c2c1 ; N#Cc1ccc2c(c1)c1ccccc1n2-c1ccc2oc3ccc(-n4c5ccccc5c5c6sc7cccnc7c6ccc54)cc3c2c1 ; N#Cc1ccc2c(c1)c1cc(C#N)ccc1n2-c1ccc2sc3ccc(-n4c5ccccc5c5ccc6oc7ccccc7c6c54)cc3c2c1]

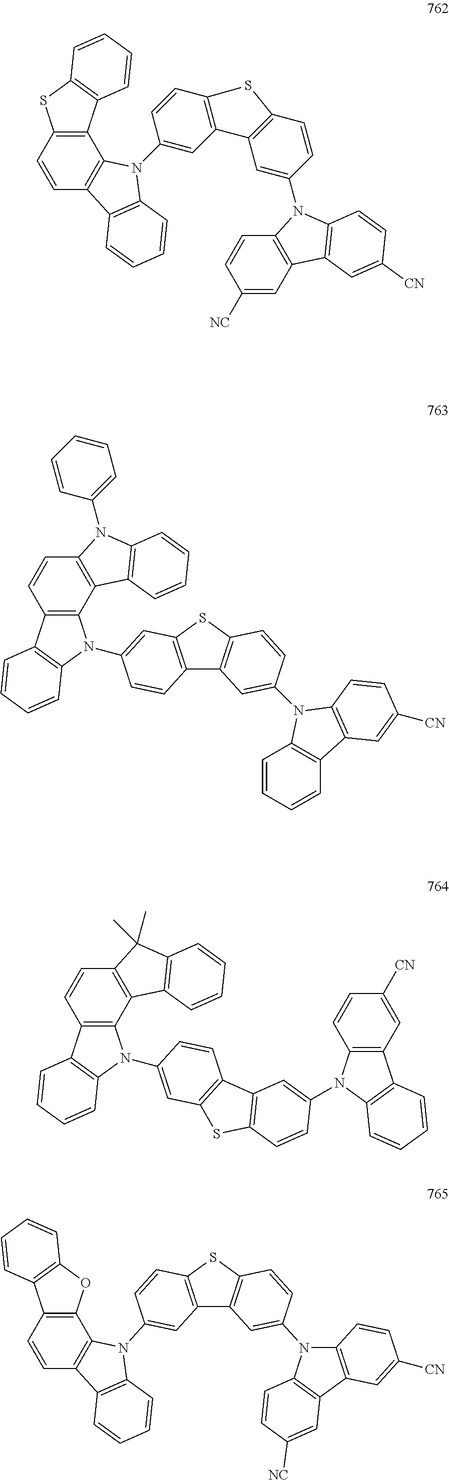 [SMILES: N#Cc1ccc2c(c1)c1cc(C#N)ccc1n2-c1ccc2sc3ccc(-n4c5ccccc5c5ccc6sc7ccccc7c6c54)cc3c2c1 ; N#Cc1ccc2c(c1)c1ccccc1n2-c1ccc2sc3cc(-n4c5ccccc5c5ccc6c(c7ccccc7n6-c6ccccc6)c54)ccc3c2c1 ; CC1(C)c2ccccc2-c2c1ccc1c3ccccc3n(-c3ccc4c(c3)sc3ccc(-n5c6ccccc6c6cc(C#N)ccc65)cc34)c21 ; N#Cc1ccc2c(c1)c1cc(C#N)ccc1n2-c1ccc2sc3ccc(-n4c5ccccc5c5ccc6c7ccccc7oc6c54)cc3c2c1]

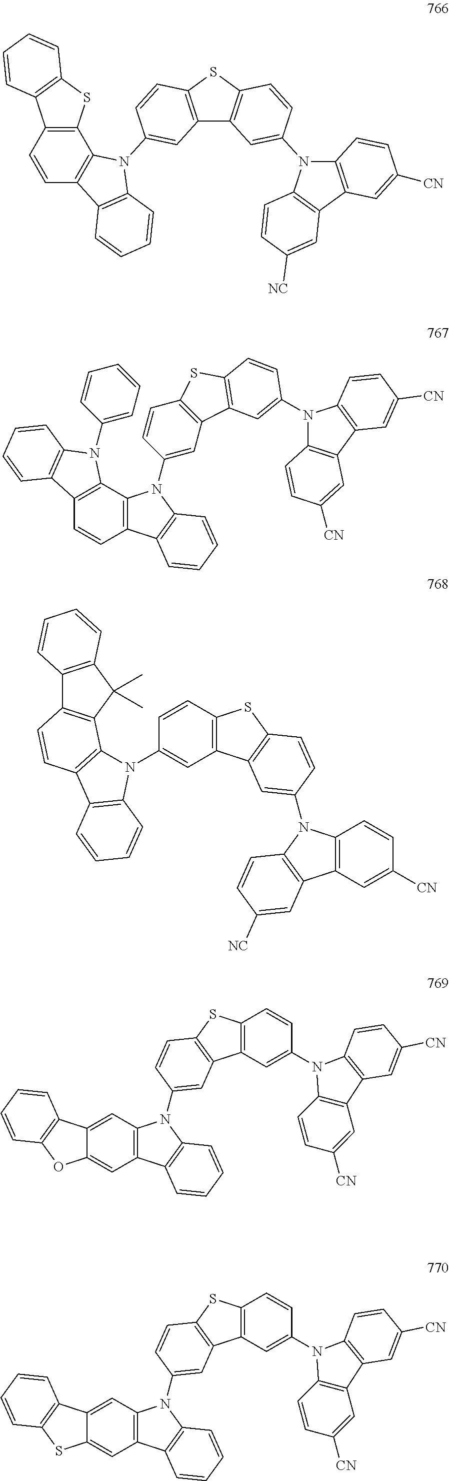 [SMILES: N#Cc1ccc2c(c1)c1cc(C#N)ccc1n2-c1ccc2sc3ccc(-n4c5ccccc5c5ccc6c7ccccc7sc6c54)cc3c2c1 ; N#Cc1ccc2c(c1)c1cc(C#N)ccc1n2-c1ccc2sc3ccc(-n4c5ccccc5c5ccc6c7ccccc7n(-c7ccccc7)c6c54)cc3c2c1 ; CC1(C)c2ccccc2-c2ccc3c4ccccc4n(-c4ccc5sc6ccc(-n7c8ccc(C#N)cc8c8cc(C#N)ccc87)cc6c5c4)c3c21 ; N#Cc1ccc2c(c1)c1cc(C#N)ccc1n2-c1ccc2sc3ccc(-n4c5ccccc5c5cc6oc7ccccc7c6cc54)cc3c2c1 ; N#Cc1ccc2c(c1)c1cc(C#N)ccc1n2-c1ccc2sc3ccc(-n4c5ccccc5c5cc6sc7ccccc7c6cc54)cc3c2c1]

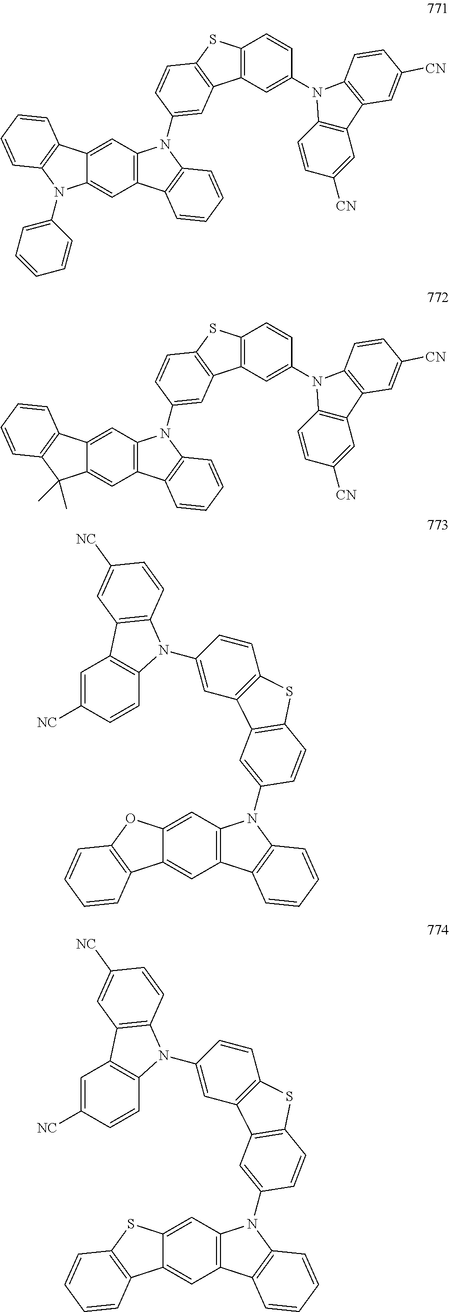

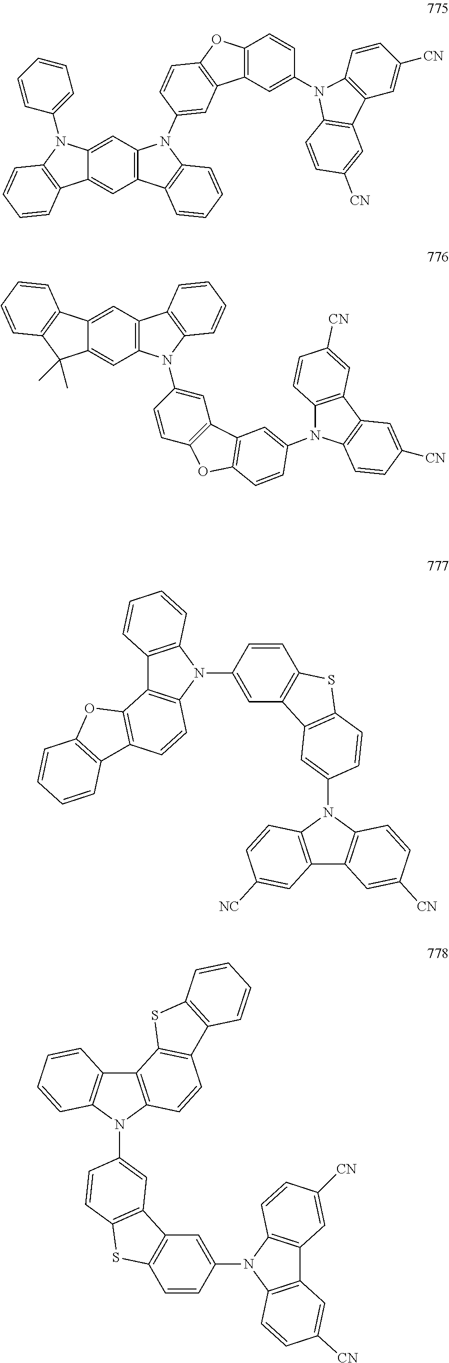 [SMILES: N#Cc1ccc2c(c1)c1cc(C#N)ccc1n2-c1ccc2oc3ccc(-n4c5ccccc5c5cc6c7ccccc7n(-c7ccccc7)c6cc54)cc3c2c1 ; CC1(C)c2ccccc2-c2cc3c4ccccc4n(-c4ccc5oc6ccc(-n7c8ccc(C#N)cc8c8cc(C#N)ccc87)cc6c5c4)c3cc21 ; N#Cc1ccc2c(c1)c1cc(C#N)ccc1n2-c1ccc2sc3ccc(-n4c5ccccc5c5c6oc7ccccc7c6ccc54)cc3c2c1 ; N#Cc1ccc2c(c1)c1cc(C#N)ccc1n2-c1ccc2sc3ccc(-n4c5ccccc5c5c6sc7ccccc7c6ccc54)cc3c2c1]

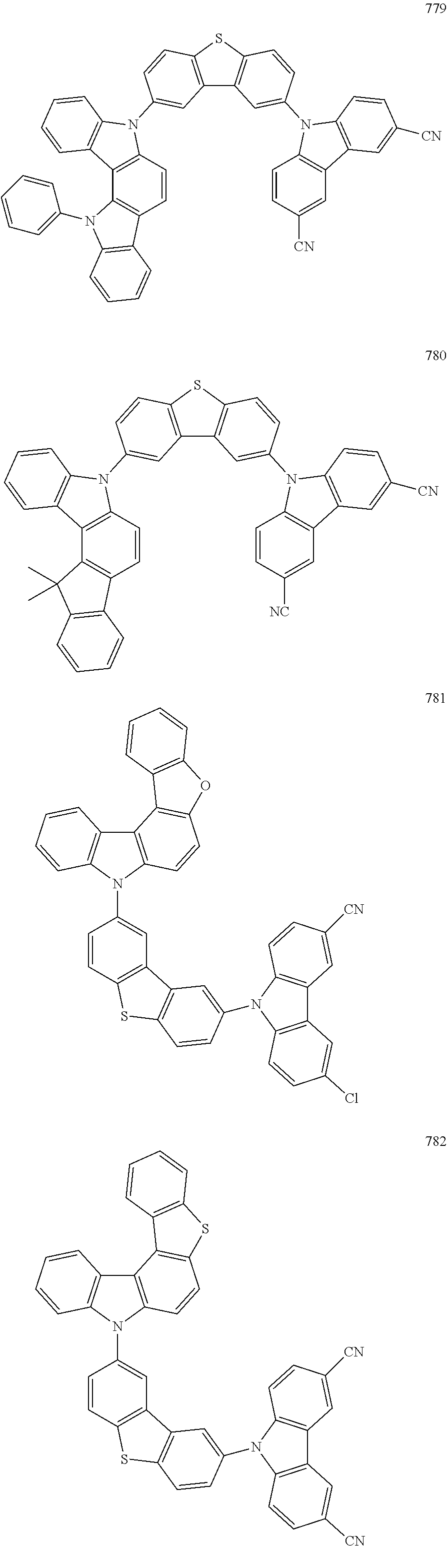 [SMILES: N#Cc1ccc2c(c1)c1cc(C#N)ccc1n2-c1ccc2sc3ccc(-n4c5ccccc5c5c4ccc4c6ccccc6n(-c6ccccc6)c45)cc3c2c1 ; CC1(C)c2ccccc2-c2ccc3c(c21)c1ccccc1n3-c1ccc2sc3ccc(-n4c5ccc(C#N)cc5c5cc(C#N)ccc54)cc3c2c1 ; N#Cc1ccc2c(c1)c1cc(Cl)ccc1n2-c1ccc2sc3ccc(-n4c5ccccc5c5c6c(ccc54)oc4ccccc46)cc3c2c1 ; N#Cc1ccc2c(c1)c1cc(C#N)ccc1n2-c1ccc2sc3ccc(-n4c5ccccc5c5c6c(ccc54)sc4ccccc46)cc3c2c1]

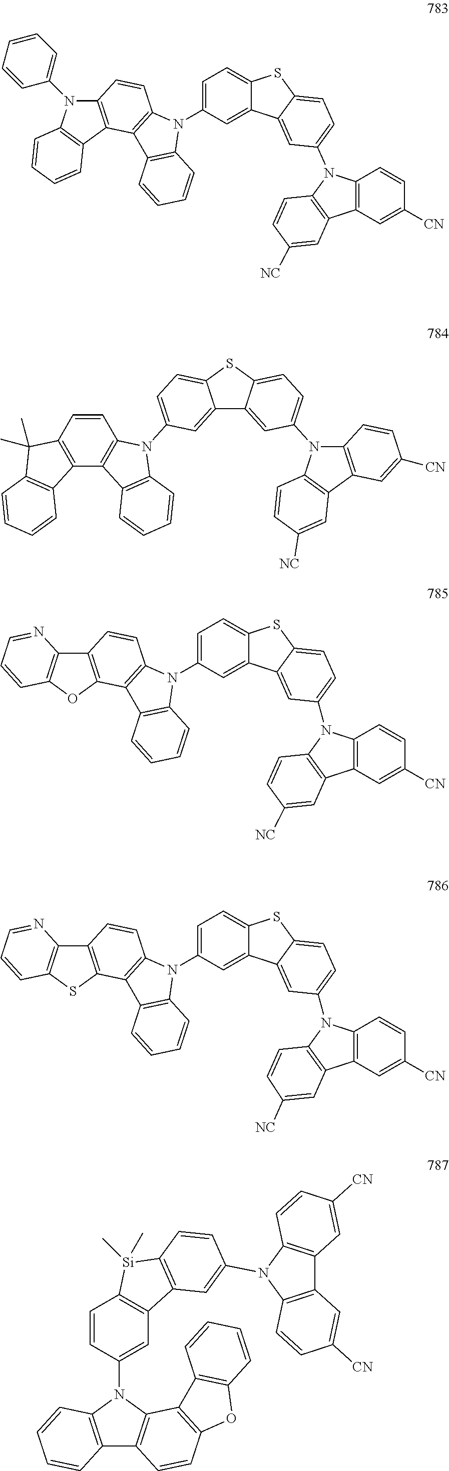 [SMILES: N#Cc1ccc2c(c1)c1cc(C#N)ccc1n2-c1ccc2sc3ccc(-n4c5ccccc5c5c6c7ccccc7n(-c7ccccc7)c6ccc54)cc3c2c1 ; CC1(C)c2ccccc2-c2c1ccc1c2c2ccccc2n1-c1ccc2sc3ccc(-n4c5ccc(C#N)cc5c5cc(C#N)ccc54)cc3c2c1 ; N#Cc1ccc2c(c1)c1cc(C#N)ccc1n2-c1ccc2sc3ccc(-n4c5ccccc5c5c6oc7cccnc7c6ccc54)cc3c2c1 ; N#Cc1ccc2c(c1)c1cc(C#N)ccc1n2-c1ccc2sc3ccc(-n4c5ccccc5c5c6sc7cccnc7c6ccc54)cc3c2c1 ; C[Si]1(C)c2ccc(-n3c4ccc(C#N)cc4c4cc(C#N)ccc43)cc2-c2cc(-n3c4ccccc4c4ccc5oc6ccccc6c5c43)ccc21]

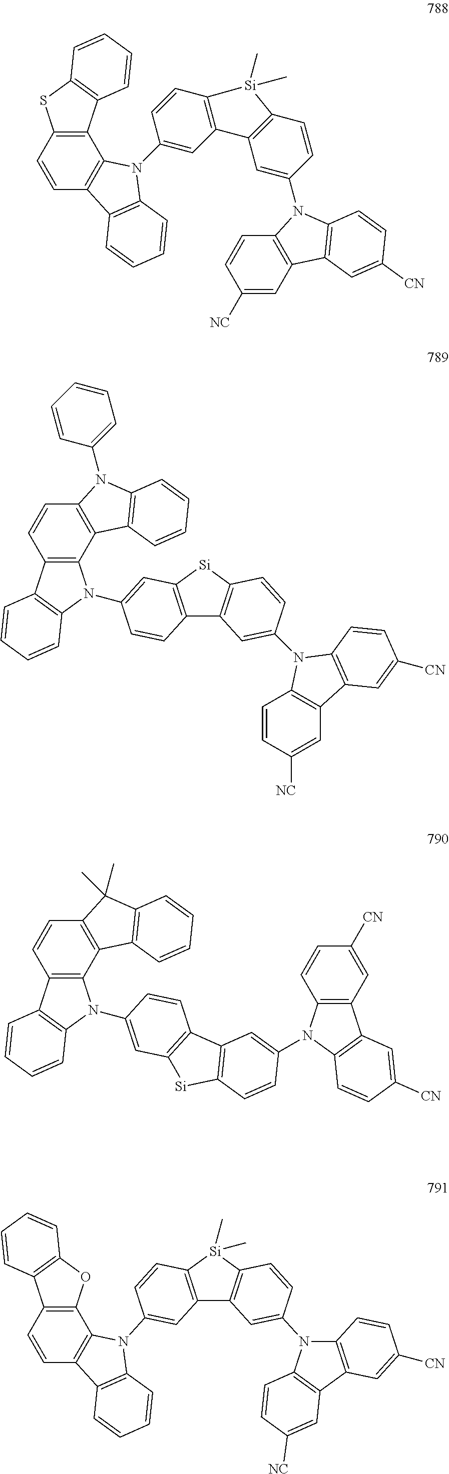

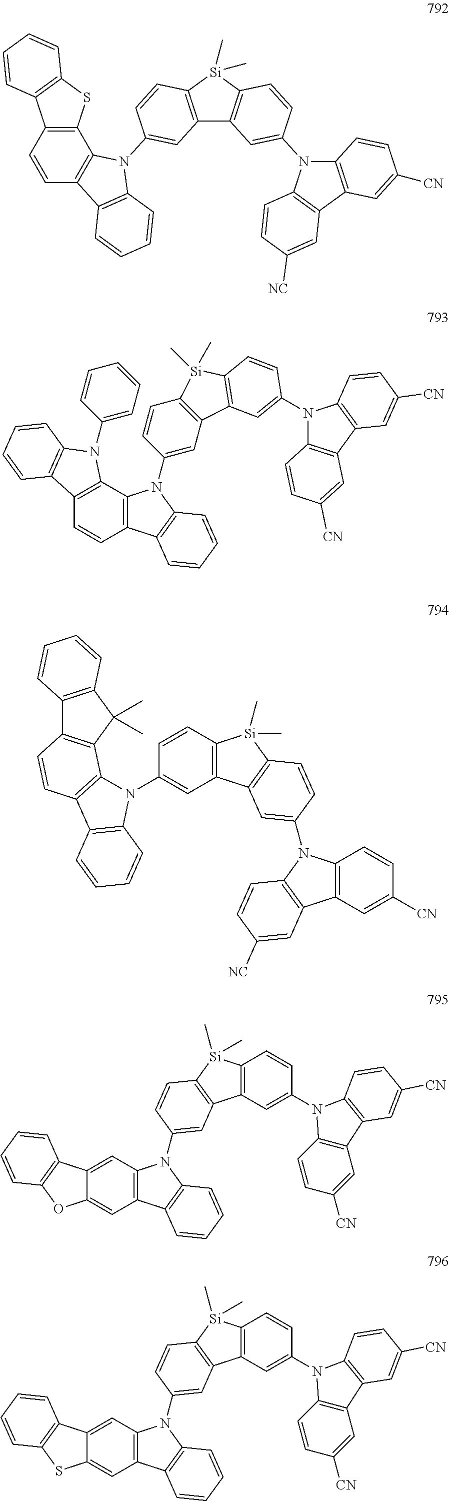 [SMILES: C[Si]1(C)c2ccc(-n3c4ccc(C#N)cc4c4cc(C#N)ccc43)cc2-c2cc(-n3c4ccccc4c4ccc5c6ccccc6sc5c43)ccc21 ; C[Si]1(C)c2ccc(-n3c4ccc(C#N)cc4c4cc(C#N)ccc43)cc2-c2cc(-n3c4ccccc4c4ccc5c6ccccc6n(-c6ccccc6)c5c43)ccc21 ; CC1(C)c2ccccc2-c2ccc3c4ccccc4n(-c4ccc5c(c4)-c4cc(-n6c7ccc(C#N)cc7c7cc(C#N)ccc76)ccc4[Si]5(C)C)c3c21 ; C[Si]1(C)c2ccc(-n3c4ccc(C#N)cc4c4cc(C#N)ccc43)cc2-c2cc(-n3c4ccccc4c4cc5oc6ccccc6c5cc43)ccc21 ; C[Si]1(C)c2ccc(-n3c4ccc(C#N)cc4c4cc(C#N)ccc43)cc2-c2cc(-n3c4ccccc4c4cc5sc6ccccc6c5cc43)ccc21]

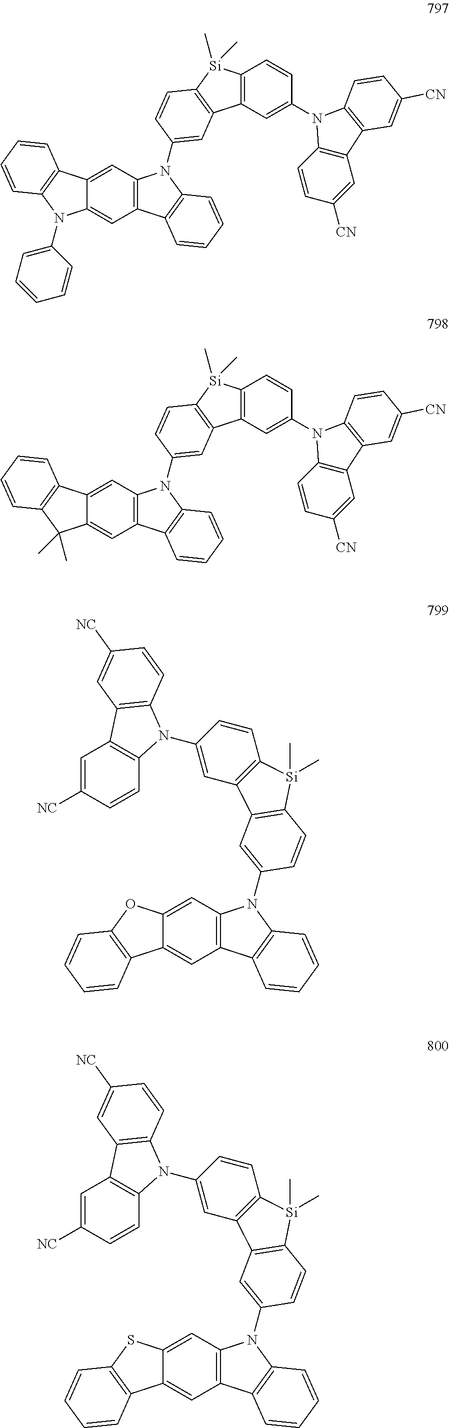 [SMILES: C[Si]1(C)c2ccc(-n3c4ccc(C#N)cc4c4cc(C#N)ccc43)cc2-c2cc(-n3c4ccccc4c4cc5c(cc43)c3ccccc3n5-c3ccccc3)ccc21 ; CC1(C)c2ccccc2-c2cc3c(cc21)c1ccccc1n3-c1ccc2c(c1)-c1cc(-n3c4ccc(C#N)cc4c4cc(C#N)ccc43)ccc1[Si]2(C)C ; C[Si]1(C)c2ccc(-n3c4ccc(C#N)cc4c4cc(C#N)ccc43)cc2-c2cc(-n3c4ccccc4c4cc5c(cc43)oc3ccccc35)ccc21 ; C[Si]1(C)c2ccc(-n3c4ccc(C#N)cc4c4cc(C#N)ccc43)cc2-c2cc(-n3c4ccccc4c4cc5c(cc43)sc3ccccc35)ccc21]

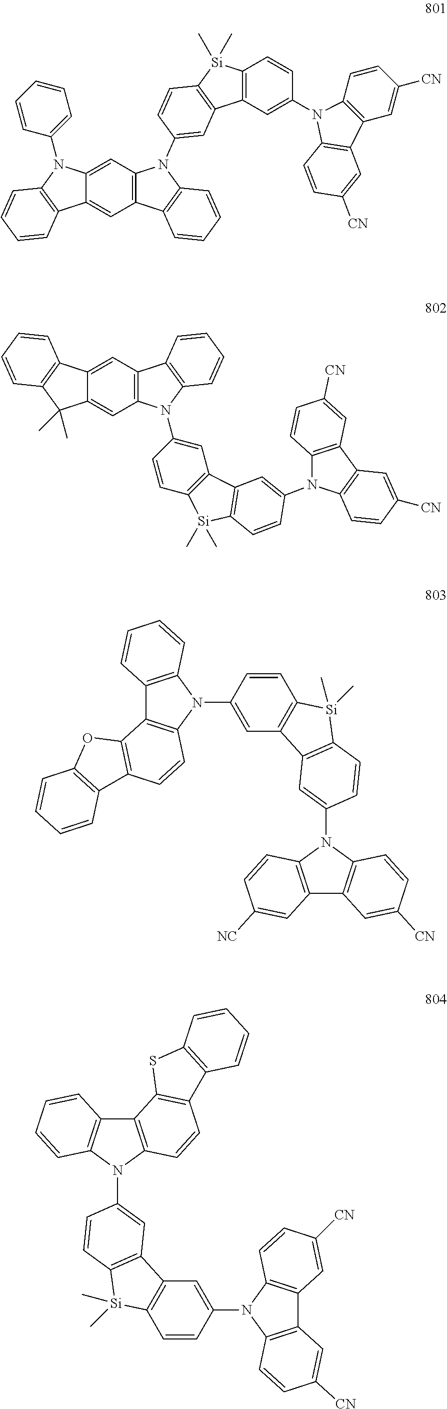 [SMILES: C[Si]1(C)c2ccc(-n3c4ccc(C#N)cc4c4cc(C#N)ccc43)cc2-c2cc(-n3c4ccccc4c4cc5c6ccccc6n(-c6ccccc6)c5cc43)ccc21 ; CC1(C)c2ccccc2-c2cc3c4ccccc4n(-c4ccc5c(c4)-c4cc(-n6c7ccc(C#N)cc7c7cc(C#N)ccc76)ccc4[Si]5(C)C)c3cc21 ; C[Si]1(C)c2ccc(-n3c4ccc(C#N)cc4c4cc(C#N)ccc43)cc2-c2cc(-n3c4ccccc4c4c5oc6ccccc6c5ccc43)ccc21 ; C[Si]1(C)c2ccc(-n3c4ccc(C#N)cc4c4cc(C#N)ccc43)cc2-c2cc(-n3c4ccccc4c4c5sc6ccccc6c5ccc43)ccc21]

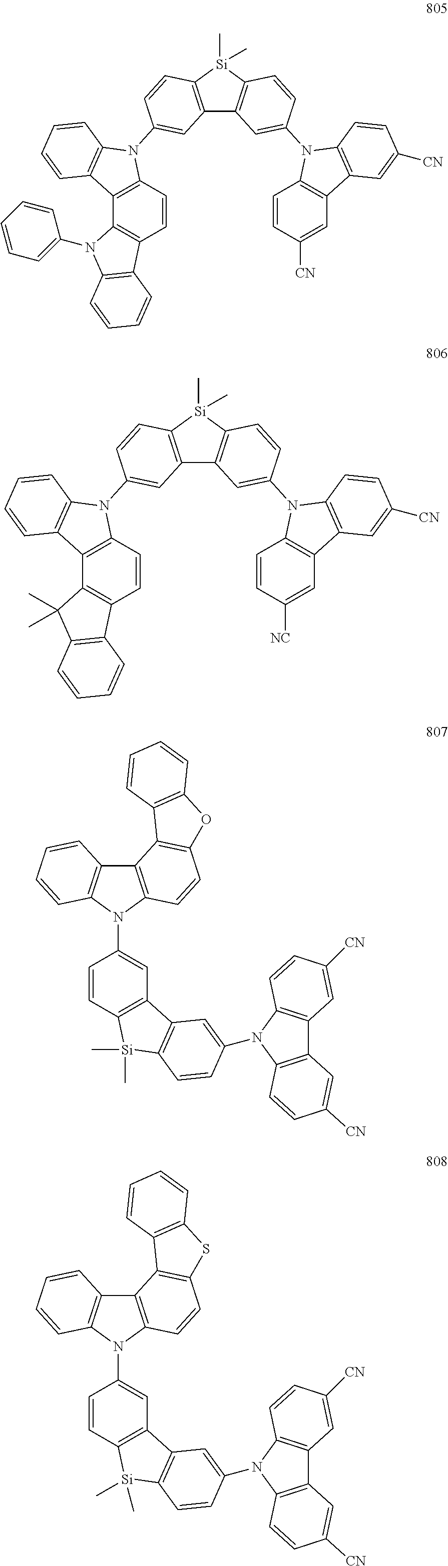 [SMILES: C[Si]1(C)c2ccc(-n3c4ccc(C#N)cc4c4cc(C#N)ccc43)cc2-c2cc(-n3c4ccccc4c4c3ccc3c5ccccc5n(-c5ccccc5)c34)ccc21 ; CC1(C)c2ccccc2-c2ccc3c(c21)c1ccccc1n3-c1ccc2c(c1)-c1cc(-n3c4ccc(C#N)cc4c4cc(C#N)ccc43)ccc1[Si]2(C)C ; C[Si]1(C)c2ccc(-n3c4ccc(C#N)cc4c4cc(C#N)ccc43)cc2-c2cc(-n3c4ccccc4c4c5c(ccc43)oc3ccccc35)ccc21 ; C[Si]1(C)c2ccc(-n3c4ccc(C#N)cc4c4cc(C#N)ccc43)cc2-c2cc(-n3c4ccccc4c4c5c(ccc43)sc3ccccc35)ccc21]

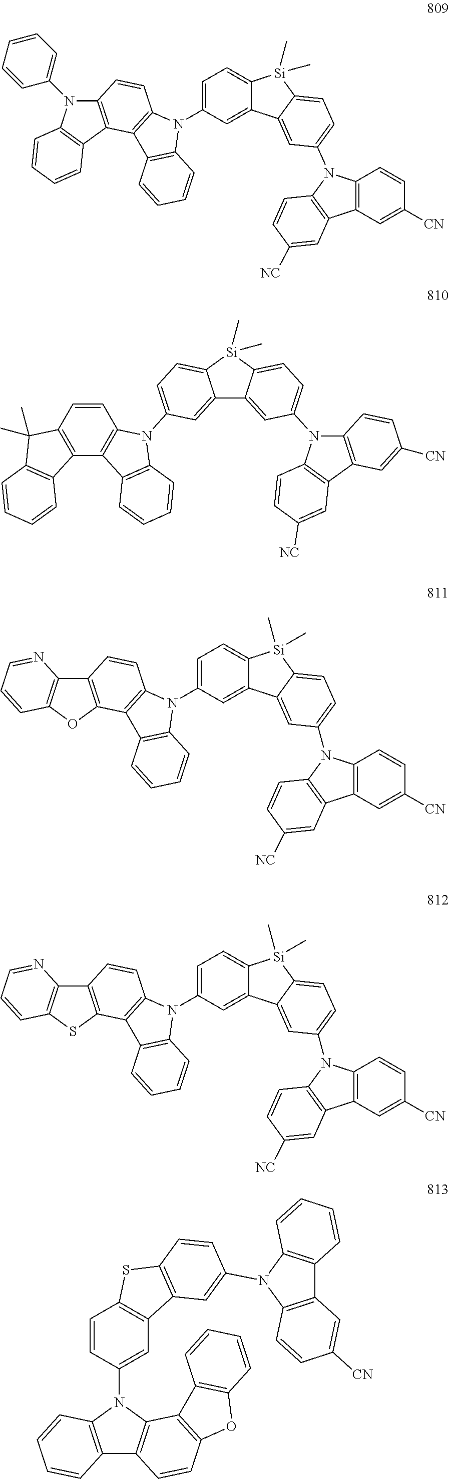 [SMILES: C[Si]1(C)c2ccc(-n3c4ccc(C#N)cc4c4cc(C#N)ccc43)cc2-c2cc(-n3c4ccccc4c4c5c6ccccc6n(-c6ccccc6)c5ccc43)ccc21 ; CC1(C)c2ccccc2-c2c1ccc1c2c2ccccc2n1-c1ccc2c(c1)-c1cc(-n3c4ccc(C#N)cc4c4cc(C#N)ccc43)ccc1[Si]2(C)C ; C[Si]1(C)c2ccc(-n3c4ccc(C#N)cc4c4cc(C#N)ccc43)cc2-c2cc(-n3c4ccccc4c4c5oc6cccnc6c5ccc43)ccc21 ; C[Si]1(C)c2ccc(-n3c4ccc(C#N)cc4c4cc(C#N)ccc43)cc2-c2cc(-n3c4ccccc4c4c5sc6cccnc6c5ccc43)ccc21 ; N#Cc1ccc2c(c1)c1ccccc1n2-c1ccc2sc3ccc(-n4c5ccccc5c5ccc6oc7ccccc7c6c54)cc3c2c1]

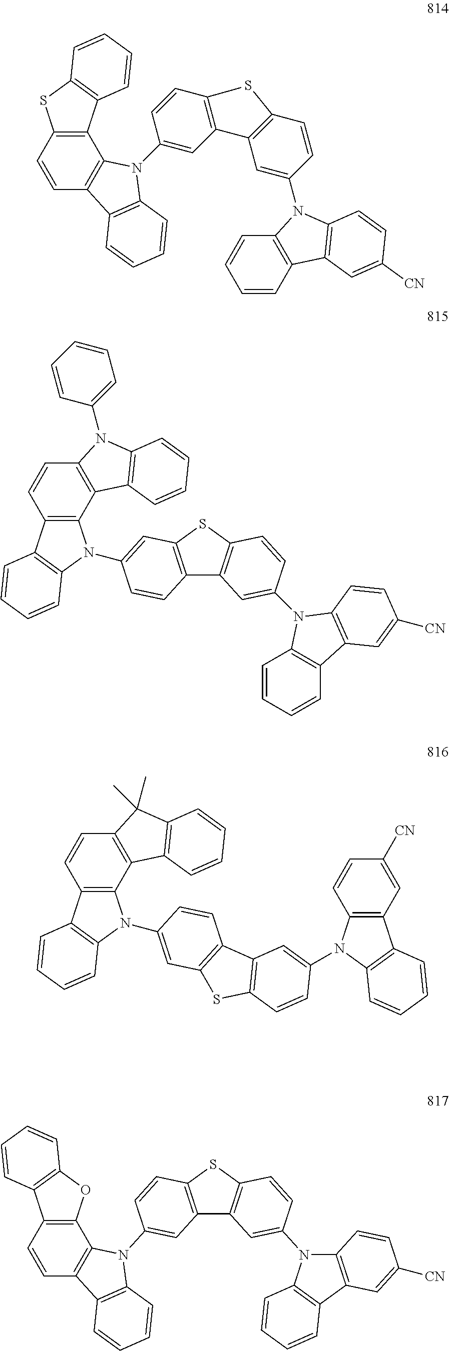 [SMILES: N#Cc1ccc2c(c1)c1ccccc1n2-c1ccc2sc3ccc(-n4c5ccccc5c5ccc6sc7ccccc7c6c54)cc3c2c1 ; N#Cc1ccc2c(c1)c1ccccc1n2-c1ccc2sc3cc(-n4c5ccccc5c5ccc6c(c7ccccc7n6-c6ccccc6)c54)ccc3c2c1 ; CC1(C)c2ccccc2-c2c1ccc1c3ccccc3n(-c3ccc4c(c3)sc3ccc(-n5c6ccccc6c6cc(C#N)ccc65)cc34)c21 ; N#Cc1ccc2c(c1)c1ccccc1n2-c1ccc2sc3ccc(-n4c5ccccc5c5ccc6c7ccccc7oc6c54)cc3c2c1]

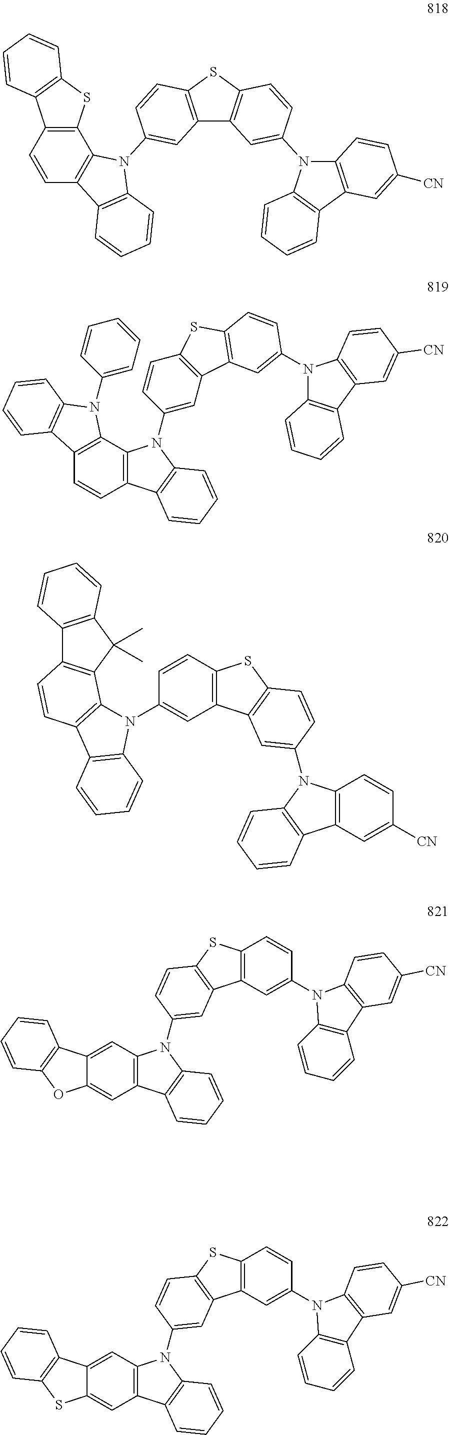 [SMILES: N#Cc1ccc2c(c1)c1ccccc1n2-c1ccc2sc3ccc(-n4c5ccccc5c5ccc6c7ccccc7sc6c54)cc3c2c1 ; N#Cc1ccc2c(c1)c1ccccc1n2-c1ccc2sc3ccc(-n4c5ccccc5c5ccc6c7ccccc7n(-c7ccccc7)c6c54)cc3c2c1 ; CC1(C)c2ccccc2-c2ccc3c4ccccc4n(-c4ccc5sc6ccc(-n7c8ccccc8c8cc(C#N)ccc87)cc6c5c4)c3c21 ; N#Cc1ccc2c(c1)c1ccccc1n2-c1ccc2sc3ccc(-n4c5ccccc5c5cc6oc7ccccc7c6cc54)cc3c2c1 ; N#Cc1ccc2c(c1)c1ccccc1n2-c1ccc2sc3ccc(-n4c5ccccc5c5cc6sc7ccccc7c6cc54)cc3c2c1]

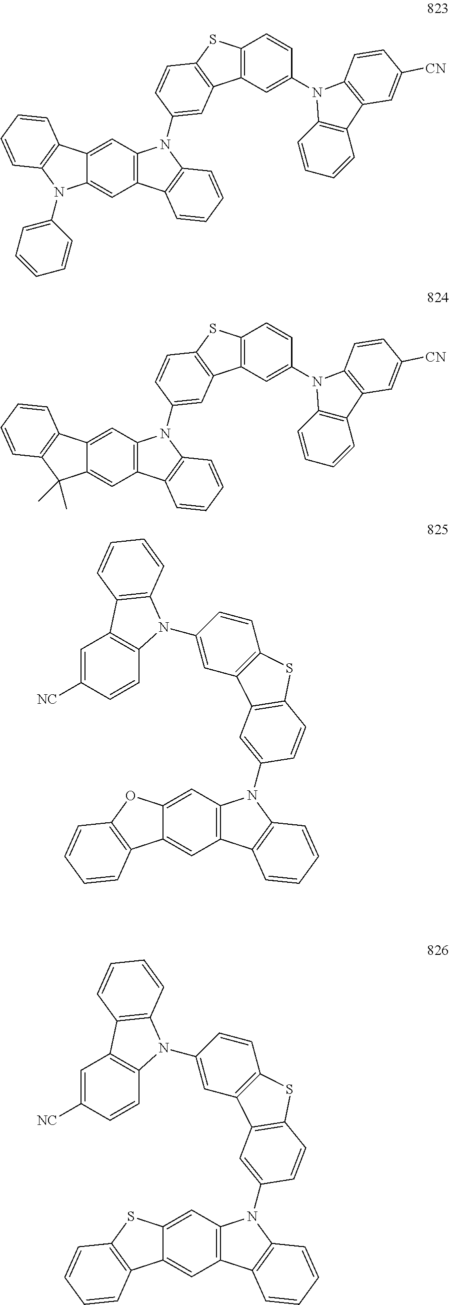 [SMILES: N#Cc1ccc2c(c1)c1ccccc1n2-c1ccc2sc3ccc(-n4c5ccccc5c5cc6c(cc54)c4ccccc4n6-c4ccccc4)cc3c2c1 ; CC1(C)c2ccccc2-c2cc3c(cc21)c1ccccc1n3-c1ccc2sc3ccc(-n4c5ccccc5c5cc(C#N)ccc54)cc3c2c1 ; N#Cc1ccc2c(c1)c1ccccc1n2-c1ccc2sc3ccc(-n4c5ccccc5c5cc6c(cc54)oc4ccccc46)cc3c2c1 ; N#Cc1ccc2c(c1)c1ccccc1n2-c1ccc2sc3ccc(-n4c5ccccc5c5cc6c(cc54)sc4ccccc46)cc3c2c1]

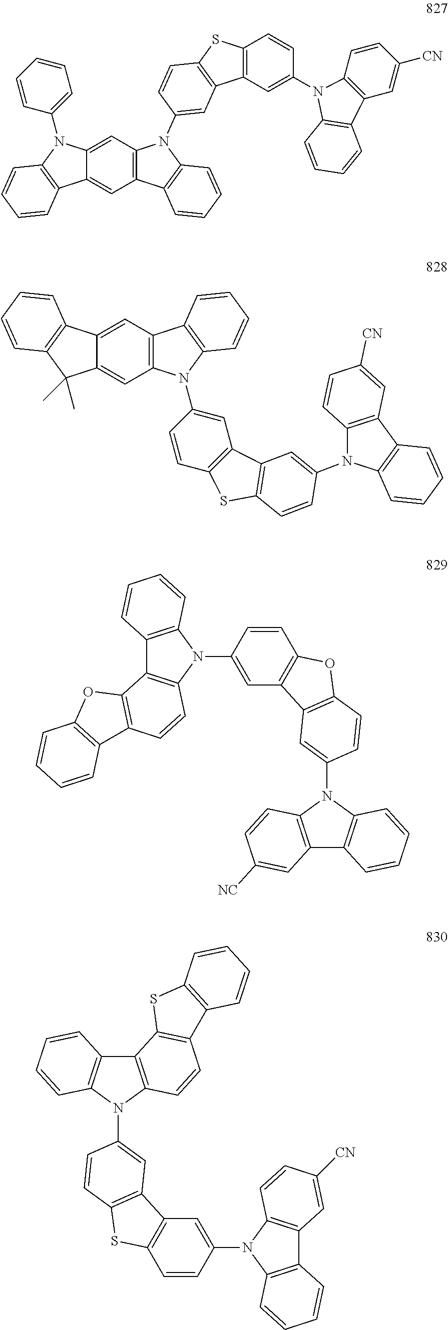 [SMILES: N#Cc1ccc2c(c1)c1ccccc1n2-c1ccc2sc3ccc(-n4c5ccccc5c5cc6c7ccccc7n(-c7ccccc7)c6cc54)cc3c2c1 ; CC1(C)c2ccccc2-c2cc3c4ccccc4n(-c4ccc5sc6ccc(-n7c8ccccc8c8cc(C#N)ccc87)cc6c5c4)c3cc21 ; N#Cc1ccc2c(c1)c1ccccc1n2-c1ccc2sc3ccc(-n4c5ccccc5c5c6oc7ccccc7c6ccc54)cc3c2c1 ; N#Cc1ccc2c(c1)c1ccccc1n2-c1ccc2sc3ccc(-n4c5ccccc5c5c6sc7ccccc7c6ccc54)cc3c2c1]

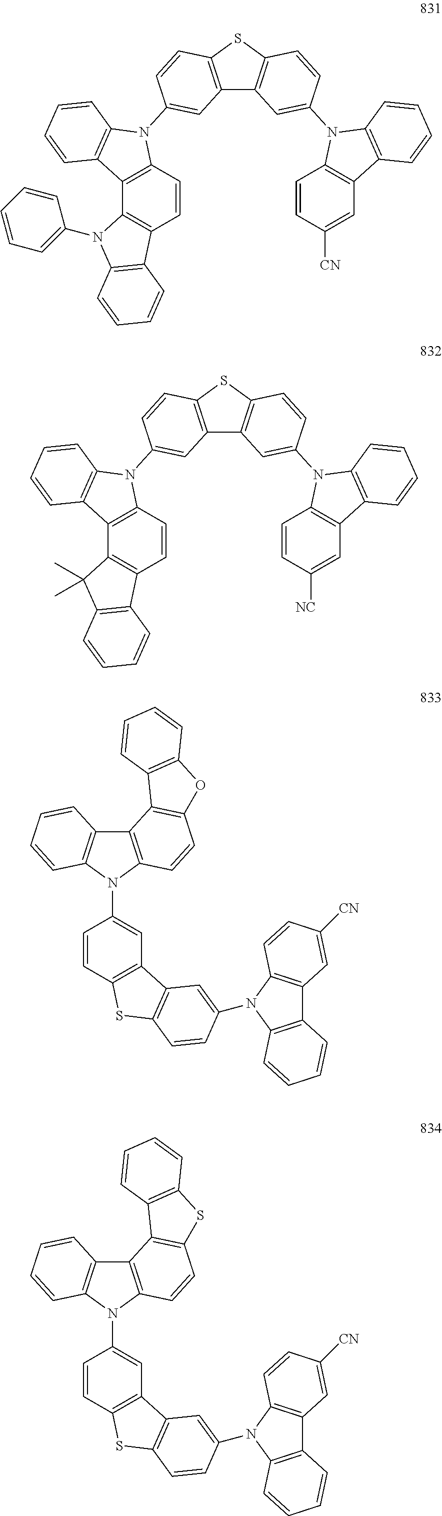 [SMILES: N#Cc1ccc2c(c1)c1ccccc1n2-c1ccc2sc3ccc(-n4c5ccccc5c5c4ccc4c6ccccc6n(-c6ccccc6)c45)cc3c2c1 ; CC1(C)c2ccccc2-c2ccc3c(c21)c1ccccc1n3-c1ccc2sc3ccc(-n4c5ccccc5c5cc(C#N)ccc54)cc3c2c1 ; N#Cc1ccc2c(c1)c1ccccc1n2-c1ccc2sc3ccc(-n4c5ccccc5c5c6c(ccc54)oc4ccccc46)cc3c2c1 ; N#Cc1ccc2c(c1)c1ccccc1n2-c1ccc2sc3ccc(-n4c5ccccc5c5c6c(ccc54)sc4ccccc46)cc3c2c1]

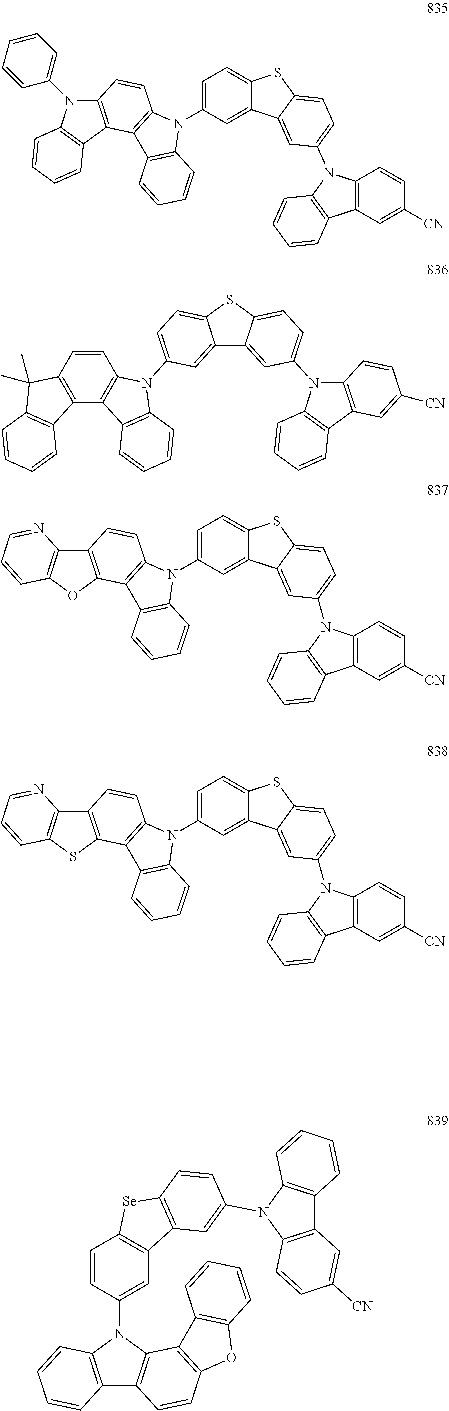 [SMILES: N#Cc1ccc2c(c1)c1ccccc1n2-c1ccc2sc3ccc(-n4c5ccccc5c5c6c7ccccc7n(-c7ccccc7)c6ccc54)cc3c2c1 ; CC1(C)c2ccccc2-c2c1ccc1c2c2ccccc2n1-c1ccc2sc3ccc(-n4c5ccccc5c5cc(C#N)ccc54)cc3c2c1 ; N#Cc1ccc2c(c1)c1ccccc1n2-c1ccc2sc3ccc(-n4c5ccccc5c5c6oc7cccnc7c6ccc54)cc3c2c1 ; N#Cc1ccc2c(c1)c1ccccc1n2-c1ccc2sc3ccc(-n4c5ccccc5c5c6sc7cccnc7c6ccc54)cc3c2c1 ; N#Cc1ccc2c(c1)c1ccccc1n2-c1ccc2[se]c3ccc(-n4c5ccccc5c5ccc6oc7ccccc7c6c54)cc3c2c1]

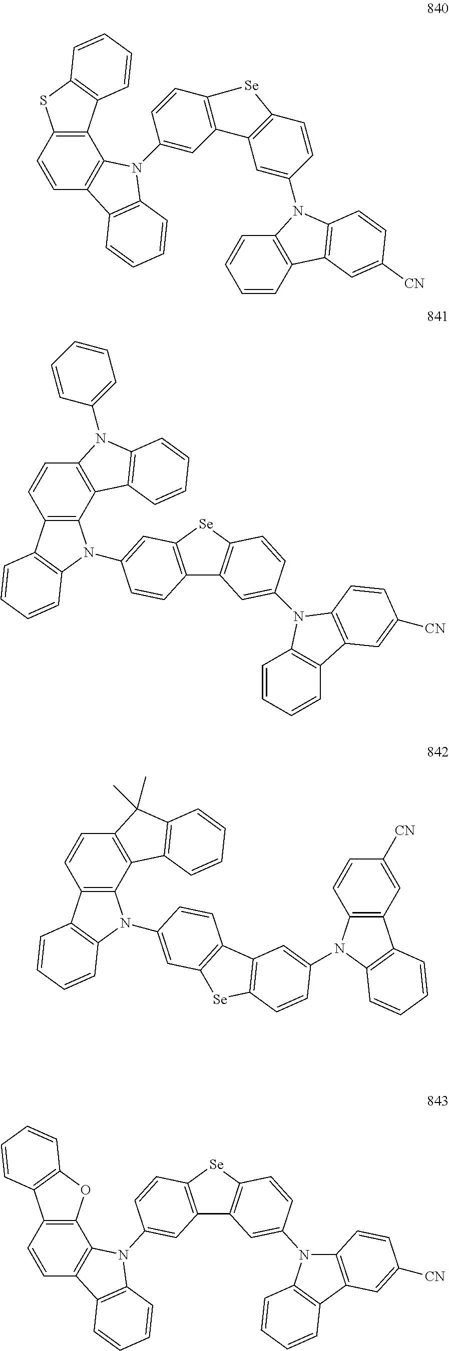 [SMILES: N#Cc1ccc2c(c1)c1ccccc1n2-c1ccc2[se]c3ccc(-n4c5ccccc5c5ccc6sc7ccccc7c6c54)cc3c2c1 ; N#Cc1ccc2c(c1)c1ccccc1n2-c1ccc2[se]c3cc(-n4c5ccccc5c5ccc6c(c7ccccc7n6-c6ccccc6)c54)ccc3c2c1 ; CC1(C)c2ccccc2-c2c1ccc1c3ccccc3n(-c3ccc4c(c3)[se]c3ccc(-n5c6ccccc6c6cc(C#N)ccc65)cc34)c21 ; N#Cc1ccc2c(c1)c1ccccc1n2-c1ccc2[se]c3ccc(-n4c5ccccc5c5ccc6c7ccccc7oc6c54)cc3c2c1]

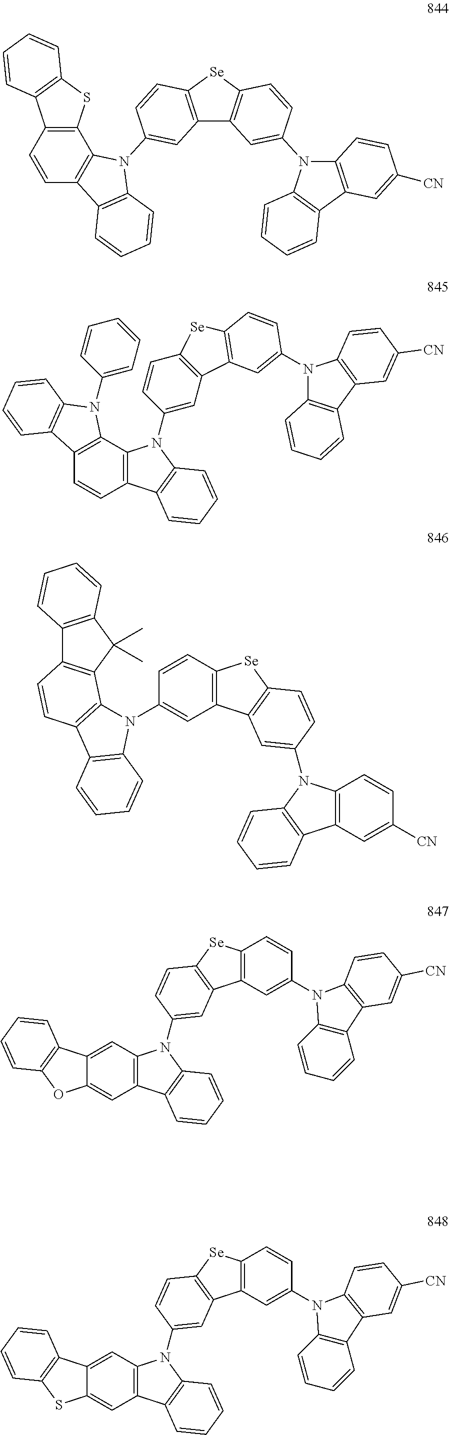 [SMILES: N#Cc1ccc2c(c1)c1ccccc1n2-c1ccc2[se]c3ccc(-n4c5ccccc5c5ccc6c7ccccc7sc6c54)cc3c2c1 ; N#Cc1ccc2c(c1)c1ccccc1n2-c1ccc2[se]c3ccc(-n4c5ccccc5c5ccc6c7ccccc7n(-c7ccccc7)c6c54)cc3c2c1 ; CC1(C)c2ccccc2-c2ccc3c4ccccc4n(-c4ccc5[se]c6ccc(-n7c8ccccc8c8cc(C#N)ccc87)cc6c5c4)c3c21 ; N#Cc1ccc2c(c1)c1ccccc1n2-c1ccc2[se]c3ccc(-n4c5ccccc5c5cc6oc7ccccc7c6cc54)cc3c2c1 ; N#Cc1ccc2c(c1)c1ccccc1n2-c1ccc2[se]c3ccc(-n4c5ccccc5c5cc6sc7ccccc7c6cc54)cc3c2c1]

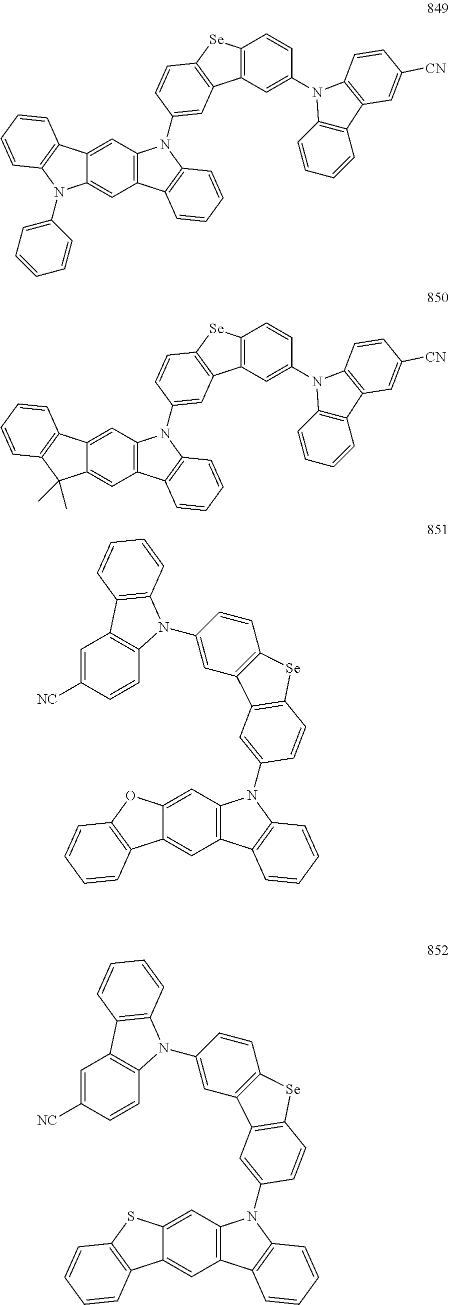 [SMILES: N#Cc1ccc2c(c1)c1ccccc1n2-c1ccc2[se]c3ccc(-n4c5ccccc5c5cc6c(cc54)c4ccccc4n6-c4ccccc4)cc3c2c1 ; CC1(C)c2ccccc2-c2cc3c(cc21)c1ccccc1n3-c1ccc2[se]c3ccc(-n4c5ccccc5c5cc(C#N)ccc54)cc3c2c1 ; N#Cc1ccc2c(c1)c1ccccc1n2-c1ccc2[se]c3ccc(-n4c5ccccc5c5cc6c(cc54)oc4ccccc46)cc3c2c1 ; N#Cc1ccc2c(c1)c1ccccc1n2-c1ccc2[se]c3ccc(-n4c5ccccc5c5cc6c(cc54)sc4ccccc46)cc3c2c1]

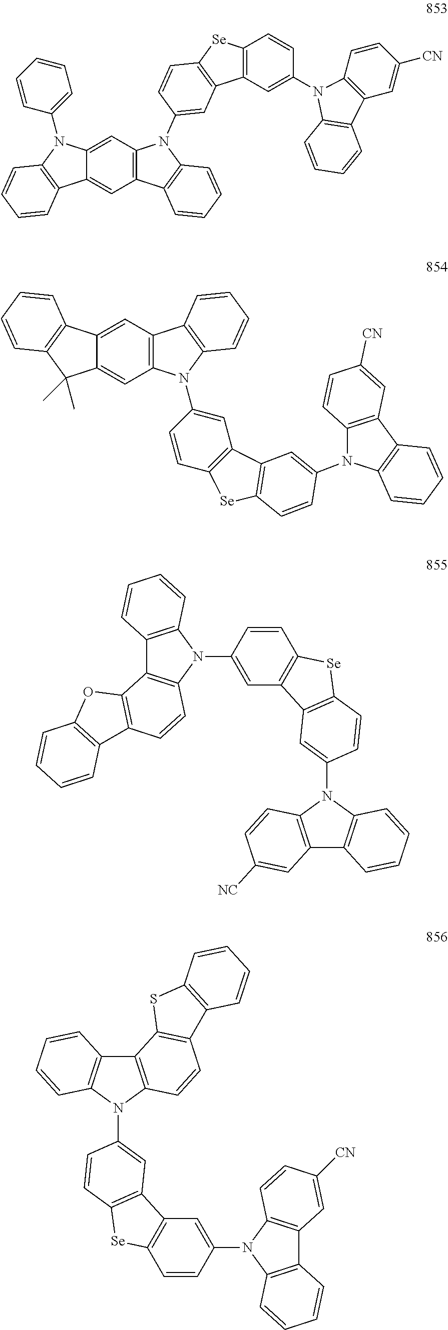 [SMILES: N#Cc1ccc2c(c1)c1ccccc1n2-c1ccc2[se]c3ccc(-n4c5ccccc5c5cc6c7ccccc7n(-c7ccccc7)c6cc54)cc3c2c1 ; CC1(C)c2ccccc2-c2cc3c4ccccc4n(-c4ccc5[se]c6ccc(-n7c8ccccc8c8cc(C#N)ccc87)cc6c5c4)c3cc21 ; N#Cc1ccc2c(c1)c1ccccc1n2-c1ccc2[se]c3ccc(-n4c5ccccc5c5c6oc7ccccc7c6ccc54)cc3c2c1 ; N#Cc1ccc2c(c1)c1ccccc1n2-c1ccc2[se]c3ccc(-n4c5ccccc5c5c6sc7ccccc7c6ccc54)cc3c2c1]

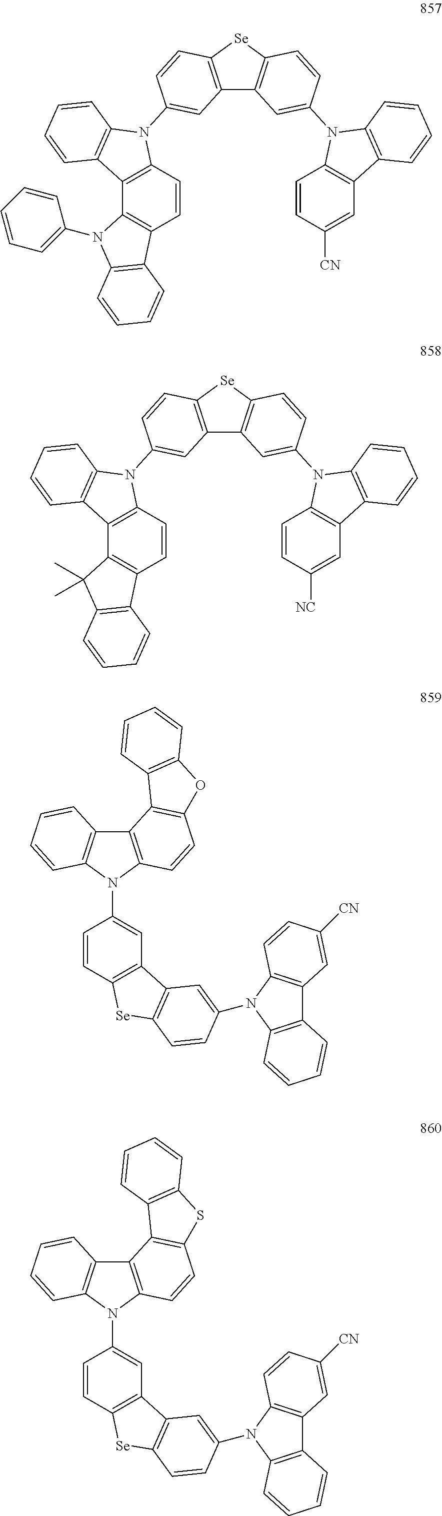 [SMILES: N#Cc1ccc2c(c1)c1ccccc1n2-c1ccc2[se]c3ccc(-n4c5ccccc5c5c4ccc4c6ccccc6n(-c6ccccc6)c45)cc3c2c1 ; CC1(C)c2ccccc2-c2ccc3c(c21)c1ccccc1n3-c1ccc2[se]c3ccc(-n4c5ccccc5c5cc(C#N)ccc54)cc3c2c1 ; N#Cc1ccc2c(c1)c1ccccc1n2-c1ccc2[se]c3ccc(-n4c5ccccc5c5c6c(ccc54)oc4ccccc46)cc3c2c1 ; N#Cc1ccc2c(c1)c1ccccc1n2-c1ccc2[se]c3ccc(-n4c5ccccc5c5c6c(ccc54)sc4ccccc46)cc3c2c1]

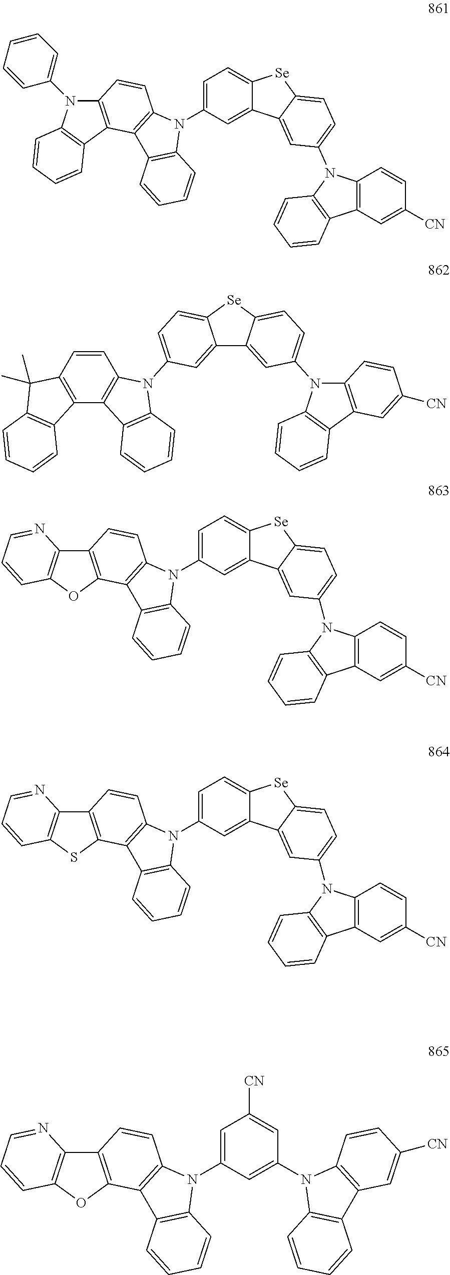 [SMILES: N#Cc1ccc2c(c1)c1ccccc1n2-c1ccc2[se]c3ccc(-n4c5ccccc5c5c6c7ccccc7n(-c7ccccc7)c6ccc54)cc3c2c1 ; CC1(C)c2ccccc2-c2c1ccc1c2c2ccccc2n1-c1ccc2[se]c3ccc(-n4c5ccccc5c5cc(C#N)ccc54)cc3c2c1 ; N#Cc1ccc2c(c1)c1ccccc1n2-c1ccc2[se]c3ccc(-n4c5ccccc5c5c6oc7cccnc7c6ccc54)cc3c2c1 ; N#Cc1ccc2c(c1)c1ccccc1n2-c1ccc2[se]c3ccc(-n4c5ccccc5c5c6sc7cccnc7c6ccc54)cc3c2c1 ; N#Cc1cc(-n2c3ccccc3c3cc(C#N)ccc32)cc(-n2c3ccccc3c3c4oc5cccnc5c4ccc32)c1]

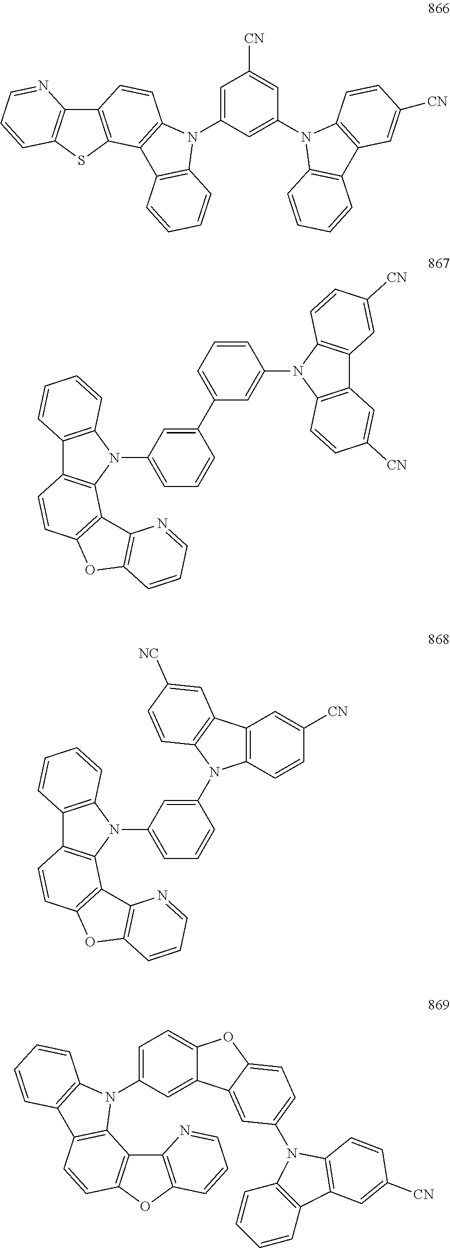 [SMILES: N#Cc1cc(-n2c3ccccc3c3cc(C#N)ccc32)cc(-n2c3ccccc3c3c4sc5cccnc5c4ccc32)c1 ; N#Cc1ccc2c(c1)c1cc(C#N)ccc1n2-c1cccc(-c2cccc(-n3c4ccccc4c4ccc5oc6cccnc6c5c43)c2)c1 ; N#Cc1ccc2c(c1)c1cc(C#N)ccc1n2-c1cccc(-n2c3ccccc3c3ccc4oc5cccnc5c4c32)c1 ; N#Cc1ccc2c(c1)c1ccccc1n2-c1ccc2oc3ccc(-n4c5ccccc5c5ccc6oc7cccnc7c6c54)cc3c2c1]

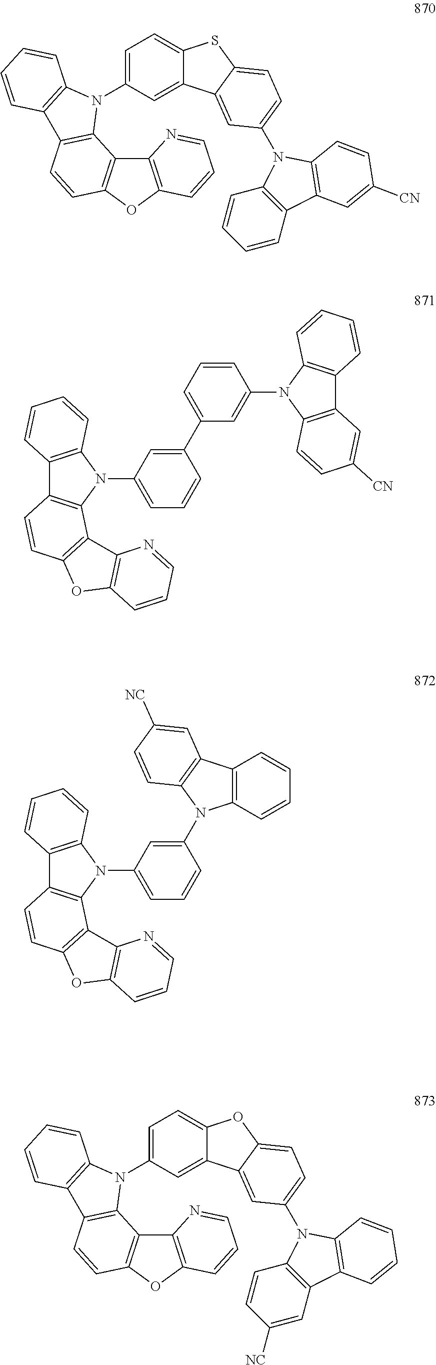 [SMILES: N#Cc1ccc2c(c1)c1ccccc1n2-c1ccc2sc3ccc(-n4c5ccccc5c5ccc6oc7cccnc7c6c54)cc3c2c1 ; N#Cc1ccc2c(c1)c1ccccc1n2-c1cccc(-c2cccc(-n3c4ccccc4c4ccc5oc6cccnc6c5c43)c2)c1 ; N#Cc1ccc2c(c1)c1ccccc1n2-c1cccc(-n2c3ccccc3c3ccc4oc5cccnc5c4c32)c1 ; N#Cc1ccc2c(c1)c1ccccc1n2-c1ccc2oc3ccc(-n4c5ccccc5c5ccc6oc7cccnc7c6c54)cc3c2c1]

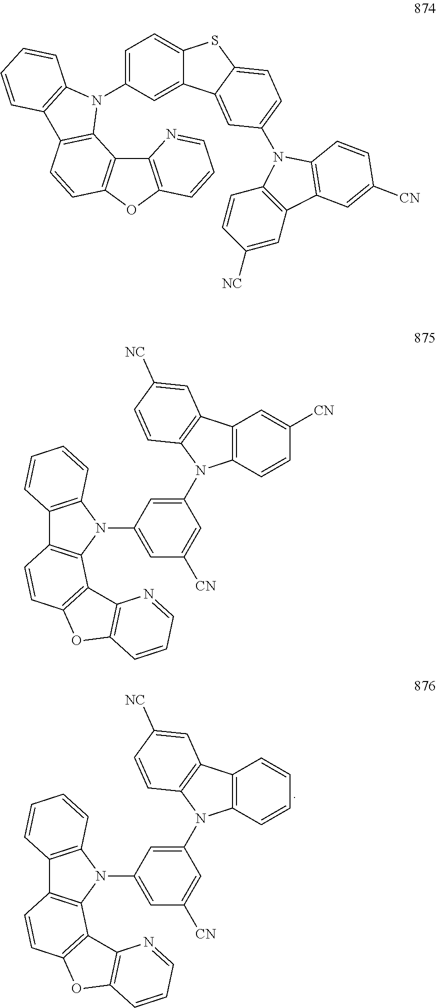

CY₁ in Formula 1 may be represented by Formula 2. Accordingly, the condensed cyclic compound represented by Formula 1 may have excellent heat resistance while maintaining high triplet energy. Also, electrical characteristics (e.g., the highest occupied molecular orbital (HOMO) energy level, the lowest unoccupied molecular orbital (LUMO) energy level, etc.) may be easily controlled to strengthen hole transport. Therefore, the condensed cyclic compound represented by Formula 1 may have a triplet (T₁) energy level and the HOMO and the LUMO energy levels suitable for use as a material for an electronic device, for example, an organic light-emitting device (e.g., a material for a host in an emission layer, a common layer, etc.).

Also, at least one selected from X₇, X₈, X₁₄, and X₂₁ to X₂₈ in the condensed cyclic compound represented by Formula 1 may be essentially C(CN). Therefore, electrical characteristics (e.g., the HOMO energy level, the LUMO energy level, etc.) of the condensed cyclic compound represented by Formula 1 may be easily controlled to strengthen charge (e.g., electron) transport and have excellent heat resistance.

For example, the HOMO energy level, the LUMO energy level, the T₁ energy level, and the singlet (S₁) energy level of Compounds 27, 29, 33, 43, 45, 93, 165, 299, 665, 876, and A to C were evaluated using a Gaussian program. Simulation results thereof are shown in Table 1:

TABLE 1

| | HOMO (eV) | LUMO (eV) | T₁ (eV) | S₁(eV) |
|---|---|---|---|---|
| Compound 27 | −5.750 | −1.768 | 2.991 | 3.612 |
| Compound 29 | −5.348 | −1.662 | 2.967 | 3.360 |
| Compound 33 | −5.503 | −1.658 | 2.904 | 3.295 |
| Compound 43 | −5.626 | −1.717 | 2.998 | 3.550 |
| Compound 44 | −5.655 | −1.730 | 2.961 | 3.573 |
| Compound 45 | −5.342 | −1.682 | 2.949 | 3.317 |
| Compound 93 | −5.825 | −1.463 | 2.978 | 3.783 |
| Compound 165 | −5.817 | −1.447 | 2.992 | 3.821 |
| Compound 299 | −5.713 | −1.651 | 2.978 | 3.585 |
| Compound 665 | −5.703 | −2.131 | 2.839 | 3.032 |
| Compound 876 | −5.885 | −1.971 | 2.936 | 3.300 |
| Compound A | −5.450 | −1.080 | 3.160 | 3.330 |
| Compound B | −5.069 | −0.770 | 2.967 | 3.356 |
| Compound C | −5.802 | −1.703 | 3.080 | 3.430 |

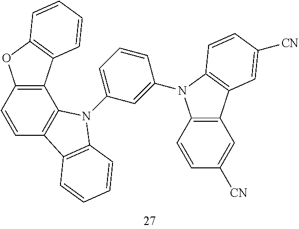 [SMILES: N#Cc1ccc2c(c1)c1cc(C#N)ccc1n2-c1cccc(-n2c3ccccc3c3ccc4oc5ccccc5c4c32)c1]

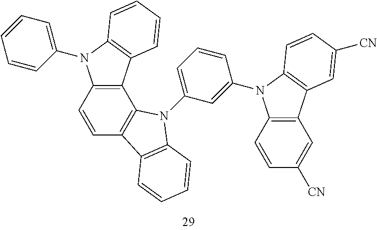 [SMILES: N#Cc1ccc2c(c1)c1cc(C#N)ccc1n2-c1cccc(-n2c3ccccc3c3ccc4c(c5ccccc5n4-c4ccccc4)c32)c1]

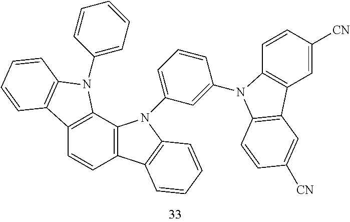 [SMILES: N#Cc1ccc2c(c1)c1cc(C#N)ccc1n2-c1cccc(-n2c3ccccc3c3ccc4c5ccccc5n(-c5ccccc5)c4c32)c1]

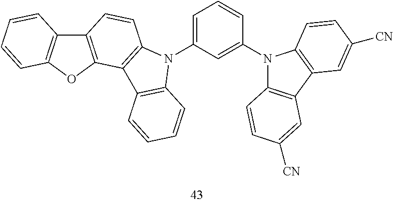 [SMILES: N#Cc1ccc2c(c1)c1cc(C#N)ccc1n2-c1cccc(-n2c3ccccc3c3c4oc5ccccc5c4ccc32)c1]

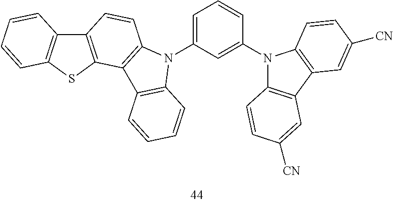 [SMILES: N#Cc1ccc2c(c1)c1cc(C#N)ccc1n2-c1cccc(-n2c3ccccc3c3c4sc5ccccc5c4ccc32)c1]

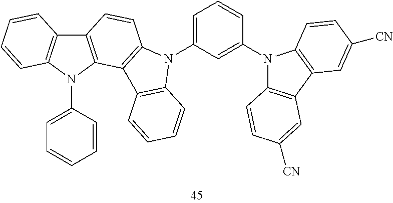 [SMILES: N#Cc1ccc2c(c1)c1cc(C#N)ccc1n2-c1cccc(-n2c3ccccc3c3c2ccc2c4ccccc4n(-c4ccccc4)c23)c1]

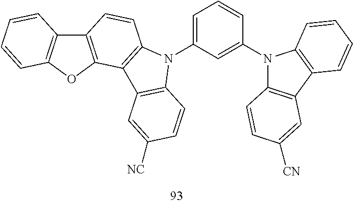 [SMILES: N#Cc1ccc2c(c1)c1ccccc1n2-c1cccc(-n2c3ccc(C#N)cc3c3c4oc5ccccc5c4ccc32)c1]

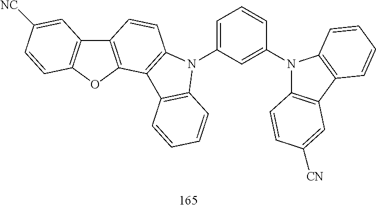 [SMILES: N#Cc1ccc2oc3c(ccc4c3c3ccccc3n4-c3cccc(-n4c5ccccc5c5cc(C#N)ccc54)c3)c2c1]

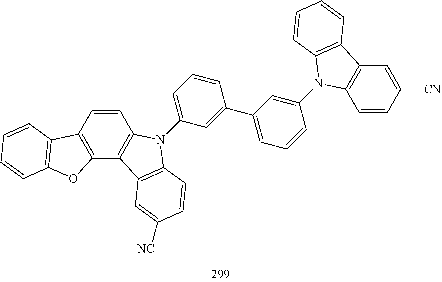 [SMILES: N#Cc1ccc2c(c1)c1ccccc1n2-c1cccc(-c2cccc(-n3c4ccc(C#N)cc4c4c5oc6ccccc6c5ccc43)c2)c1]

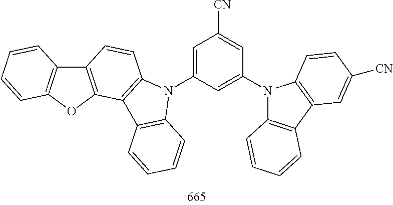 [SMILES: N#Cc1cc(-n2c3ccccc3c3cc(C#N)ccc32)cc(-n2c3ccccc3c3c4oc5ccccc5c4ccc32)c1]

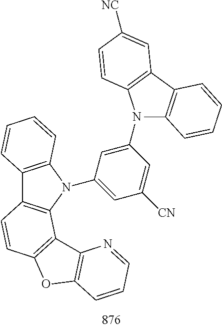 [SMILES: N#Cc1cc(-n2c3ccccc3c3cc(C#N)ccc32)cc(-n2c3ccccc3c3ccc4oc5cccnc5c4c32)c1]

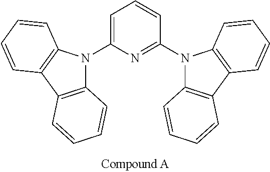 [SMILES: c1cc(-n2c3ccccc3c3ccccc32)nc(-n2c3ccccc3c3ccccc32)c1]

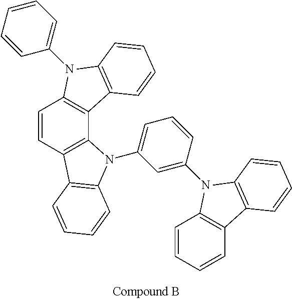 [SMILES: c1ccc(-n2c3ccccc3c3c2ccc2c4ccccc4n(-c4cccc(-n5c6ccccc6c6ccccc65)c4)c23)cc1]

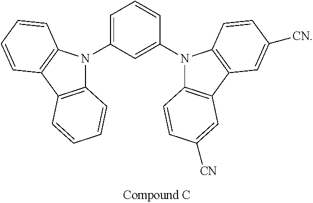 [SMILES: N#Cc1ccc2c(c1)c1cc(C#N)ccc1n2-c1cccc(-n2c3ccccc3c3ccccc32)c1]

Referring to Table 1, it was confirmed that the condensed cyclic compound represented by Formula 1 had excellent electrical characteristics, for example, a high T₁ energy level.

Synthesis methods of the condensed cyclic compound represented by Formula 1 may be understood by one of ordinary skill in the art by referring to Synthesis Examples provided below.

The condensed cyclic compound represented by Formula 1 is suitable for use in an organic layer of an organic light-emitting device, for example, for use as a material for forming an emission layer and/or an electron/hole transport region.

Thus, another aspect provides an organic light-emitting device that includes:

a first electrode;

a second electrode; and

an organic layer that is disposed between the first electrode and the second electrode,

wherein the organic layer includes an emission layer, and

wherein the organic layer may include at least one of the condensed cyclic compounds represented by Formula 1.

The organic light-emitting device may have, due to the inclusion of the organic layer including the condensed cyclic compound represented by Formula 1, high efficiency and a long lifespan.

In one or more embodiments, the condensed cyclic compound represented by Formula 1 may be included in the emission layer.

In one or more embodiments, the condensed cyclic compound represented by Formula 1 may be included in the emission layer, and the condensed cyclic compound represented by Formula 1 may be a delayed fluorescent material.

In one or more embodiments, the emission layer may include a host and a dopant (an amount of the host may be larger than an amount of the dopant), and the host may include the condensed cyclic compound represented by Formula 1. The condensed cyclic compound acting as the host may transfer energy to the dopant by a delayed fluorescence emission mechanism. The dopant may include at least one selected from a fluorescent dopant and a phosphorescent dopant. The dopant may be selected from dopants known in the related art. The host may further include any host selected from hosts known in the related art.

In one or more embodiments, the emission layer may include a host and a dopant (an amount of the host may be larger than an amount of the dopant), and the dopant may include the condensed cyclic compound represented by Formula 1. The condensed cyclic compound acting as the dopant may emit a delayed fluorescence by a delayed fluorescence emission mechanism. The host may be selected from dopants known in the related art.

The emission layer may emit red, green, or blue light.

In one or more embodiments, the emission layer may be a blue emission layer including a phosphorescent dopant, but is not limited thereto.

In one or more embodiments, the condensed cyclic compound represented by Formula 1 may be included in the electron transport region.

In one or more embodiments, the electron transport region of the organic light-emitting device may include at least one of a hole blocking layer and an electron transport layer, and at least one of an electron blocking layer and an electron transport layer may include the condensed cyclic compound represented by Formula 1.

In one or more embodiments, the electron transport region of the organic light-emitting device may include the hole blocking layer, and the condensed cyclic compound represented by Formula 1 may be included in the hole blocking layer. The hole blocking layer may directly contact the emission layer.

The expression “(an organic layer) includes at least one of the condensed cyclic compounds” as used herein may mean that “(an organic layer) may include one condensed cyclic compound belonging to the category of Formula 1, or may include two or more different condensed cyclic compounds belonging to the category of Formula 1.”

For example, the organic layer may include, as the condensed cyclic compound, Compound 1 alone. In this embodiment, Compound 1 may be included in an emission layer of the organic light-emitting device. In one or more embodiments, the organic layer may include, as the condensed cyclic compound, Compound 1 and Compound 2. In this embodiment, Compound 1 and Compound 2 may be included in an identical layer (for example, Compound 1 and Compound 2 may all be included in an emission layer), or different layers (for example, Compound 1 may be included in an emission layer and Compound 2 may be included in a hole blocking layer).

The first electrode may be an anode, which is a hole injection electrode, and the second electrode may be a cathode, which is an electron injection electrode; or the first electrode may be a cathode, which is an electron injection electrode, and the second electrode may be an anode, which is a hole injection electrode.

For example, in the organic light-emitting device,

the first electrode may be an anode, and the second electrode may be a cathode, and

the organic layer may include a hole transport region disposed between the first electrode and the emission layer and may also include an electron transport region disposed between the emission layer and the second electrode, wherein

the hole transport region may include at least one layer selected from a hole injection layer, a hole transport layer, and an electron blocking layer, and

the electron transport region may include at least one selected from a hole blocking layer, an electron transport layer, and an electron injection layer.

The term “organic layer” as used herein refers to a single layer and/or a plurality of layers disposed between the first electrode and the second electrode of the organic light-emitting device. The “organic layer” may include, in addition to an organic compound, an organometallic complex including metal.

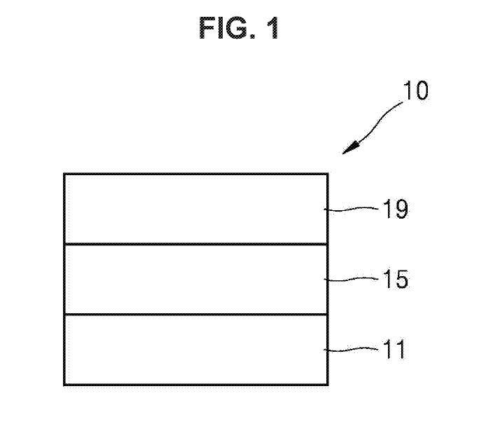

FIG. 1 is a schematic view of an organic light-emitting device 10 according to an embodiment. Hereinafter, the structure of an organic light-emitting device according to an embodiment and a method of manufacturing an organic light-emitting device according to an embodiment will be described in connection with FIG. 1. The organic light-emitting device 10 includes a first electrode 11, an organic layer 15, and a second electrode 19, which are sequentially stacked.

A substrate may be additionally disposed under the first electrode 11 or above the second electrode 19. For use as the substrate, any substrate that is used in general organic light-emitting devices may be used, and the substrate may be a glass substrate or a transparent plastic substrate, each having excellent mechanical strength, thermal stability, transparency, surface smoothness, ease of handling, and water resistance.

In one or more embodiments, the first electrode 11 may be formed by depositing or sputtering a material for forming the first electrode 11 on the substrate. The first electrode 11 may be an anode. The material for forming the first electrode 11 may be selected from materials with a high work function to facilitate hole injection. The first electrode 11 may be a reflective electrode, a semi-transmissive electrode, or a transmissive electrode. The material for forming the first electrode 11 may be an indium tin oxide (ITO), indium zinc oxide (IZO), tin oxide (SnO₂), or zinc oxide (ZnO). In one or more embodiments, the material for forming the first electrode 11 may be metal, such as magnesium (Mg), aluminum (Al), aluminum-lithium (Al—Li), calcium (Ca), magnesium-indium (Mg—In), or magnesium-silver (Mg—Ag).

The first electrode 11 may have a single-layered structure or a multi-layered structure including two or more layers. For example, the first electrode 11 may have a three-layered structure of ITO/Ag/ITO, but the structure of the first electrode 11 is not limited thereto.

The organic layer 15 may be disposed on the first electrode 11.

The organic layer 15 may include a hole transport region, an emission layer, and an electron transport region.

The hole transport region may be disposed between the first electrode 11 and the emission layer.

The hole transport region may include at least one selected from a hole injection layer, a hole transport layer, an electron blocking layer, and a buffer layer.

The hole transport region may include only either a hole injection layer or a hole transport layer. In one or more embodiments, the hole transport region may have a hole injection layer/hole transport layer structure or a hole injection layer/hole transport layer/electron blocking layer structure, which are sequentially stacked in this stated order on top of the first electrode 11.

When the hole transport region includes a hole injection layer (HIL), the hole injection layer may be formed on the first electrode 11 by using one or more suitable methods, for example, vacuum deposition, spin coating, casting, and/or Langmuir-Blodgett (LB) deposition.

When the hole injection layer is formed using vacuum deposition, vacuum deposition conditions may vary according to the compound that is used to form the hole injection layer, and the desired structure and thermal properties of the hole injection layer to be formed. For example, vacuum deposition may be performed at a temperature of about 100° C. to about 500° C., a pressure of about 10⁻⁸ torr to about 10⁻³ torr, and a deposition rate of about 0.01 to about 100 Angstroms per second (A/sec). However, the deposition conditions are not limited thereto.

When the hole injection layer is formed using spin coating, the coating conditions may vary depending on the compound that is used to form the hole injection layer, and the desired structure and thermal properties of the hole injection layer to be formed. For example, the coating rate may be in the range of about 2,000 revolutions per minute (rpm) to about 5,000 rpm, and a temperature at which heat treatment is performed to remove a solvent after coating may be in the range of about 80° C. to about 200° C. However, the coating conditions are not limited thereto.

Conditions for forming a hole transport layer and an electron blocking layer may be understood by referring to conditions for forming the hole injection layer.

The hole transport region may include at least one selected from m-MTDATA, TDATA, 2-TNATA, NPB, R-NPB, TPD, Spiro-TPD, Spiro-NPB, methylated-NPB, TAPC, HMTPD, 4,4′,4″-tris(N-carbazolyl)triphenylamine (TCTA), polyaniline/dodecylbenzene sulfonic acid (PANI/DBSA), poly(3,4-ethylenedioxythiophene)/poly(4-styrene sulfonate) (PEDOT/PSS), polyaniline/camphor sulfonic acid (PANI/CSA), polyaniline/poly(4-styrene sulfonate) (PANI/PSS), a compound represented by Formula 201 below, and a compound represented by Formula 202:

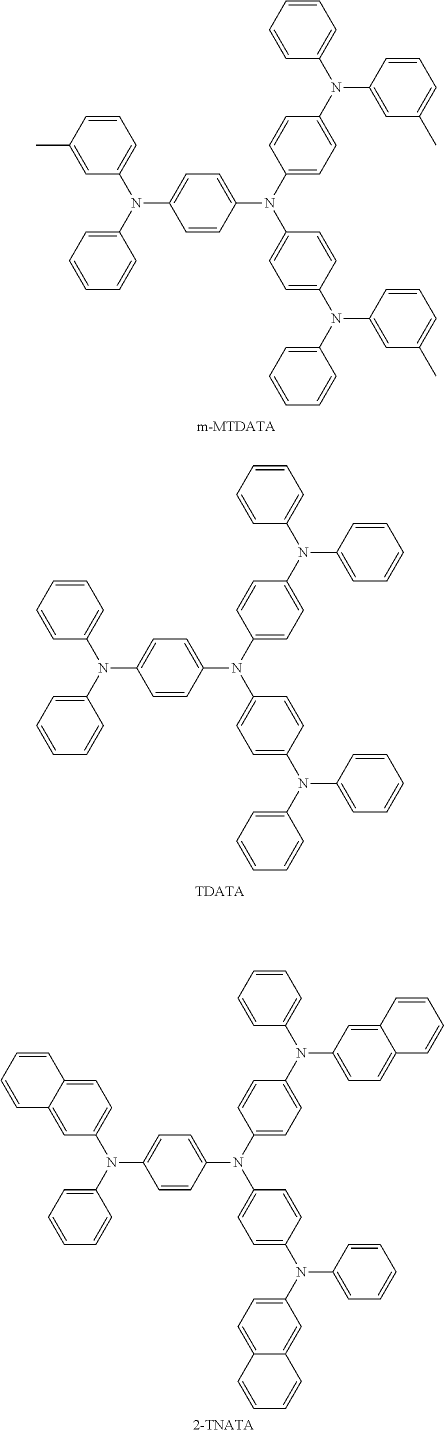

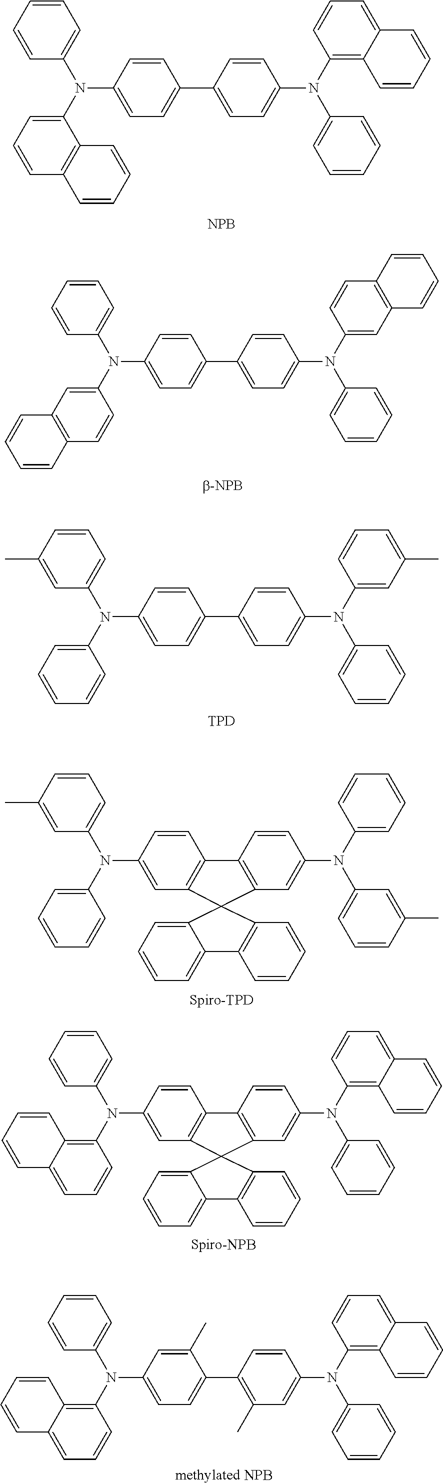

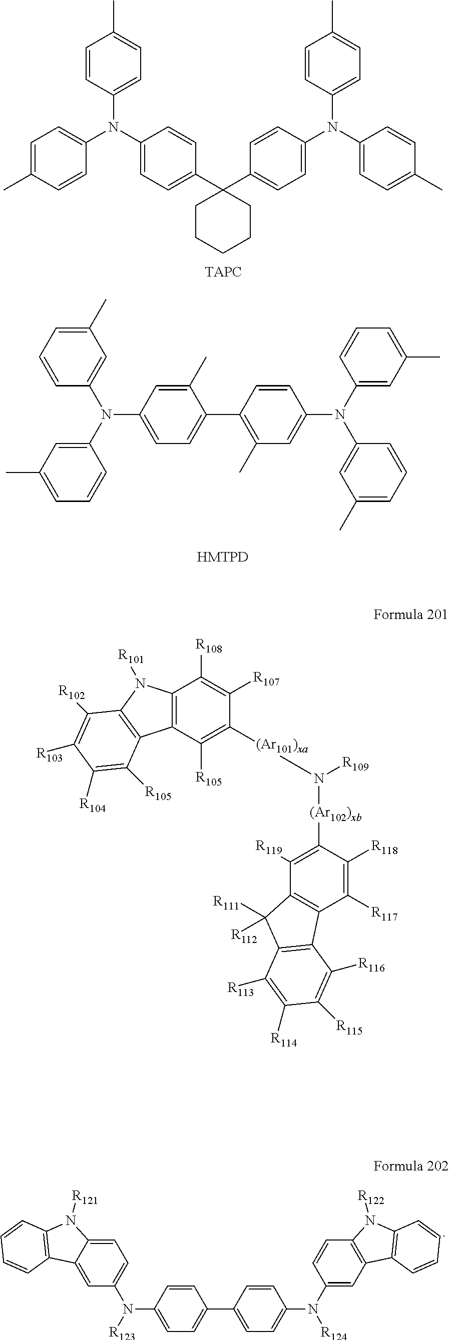

Ar₁₀₁ to Ar₁₀₂ in Formula 201 may each independently be selected from:

a phenylene group, a pentalenylene group, an indenylene group, a naphthylene group, an azulenylene group, a heptalenylene group, an acenaphthylene group, a fluorenylene group, a phenalenylene group, a phenanthrenylene group, an anthracenylene group, a fluoranthenylene group, a triphenylenylene group, a pyrenylene group, a chrysenylenylene group, a naphthacenylene group, a picenylene group, a perylenylene group, and a pentacenylene group; and

a phenylene group, a pentalenylene group, an indenylene group, a naphthylene group, an azulenylene group, a heptalenylene group, an acenaphthylene group, a fluorenylene group, a phenalenylene group, a phenanthrenylene group, an anthracenylene group, a fluoranthenylene group, a triphenylenylene group, a pyrenylene group, a chrysenylenylene group, a naphthacenylene group, a picenylene group, a perylenylene group, and a pentacenylene group, each substituted with at least one selected from deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₃-C₁₀ cycloalkenyl group, a C₁-C₁₀ heterocycloalkyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, and a monovalent non-aromatic condensed heteropolycyclic group.

xa and xb in Formula 201 may each independently be an integer selected from 0 to 5, or 0, 1, or 2. For example, xa may be 1 and xb may be 0, but xa and xb are not limited thereto.

R₁₀₁ to R₁₀₈, R₁₁₁ to R₁₁₉, and R₁₂₁ to R₁₂₄ in Formulae 201 and 202 may each independently be selected from:

hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₁₀ alkyl group (for example, a methyl group, an ethyl group, a propyl group, a butyl group, pentyl group, a hexyl group, etc.) and C₁-C₁₀ alkoxy group (for example, a methoxy group, an ethoxy group, a propoxy group, a butoxy group, a pentoxy group, etc.);

a C₁-C₁₀ alkyl group and a C₁-C₁₀ alkoxy group, each substituted with at least one selected from deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, and a phosphoric acid group or a salt thereof;

a phenyl group, a naphthyl group, an anthracenyl group, a fluorenyl group, and a pyrenyl group; and

a phenyl group, a naphthyl group, an anthracenyl group, a fluorenyl group, and a pyrenyl group, each substituted with at least one selected from deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₁₀ alkyl group, and a C₁-C₁₀ alkoxy group,

but embodiments of the present disclosure are not limited thereto.

R₁₀₉ in Formula 201 may be selected from:

a phenyl group, a naphthyl group, an anthracenyl group, and a pyridinyl group; and

a phenyl group, a naphthyl group, an anthracenyl group, and a pyridinyl group, each substituted with at least one selected from deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a phenyl group, a naphthyl group, an anthracenyl group, and a pyridinyl group.

In one or more embodiments, the compound represented by Formula 201 may be represented by Formula 201A, but embodiments of the present disclosure are not limited thereto:

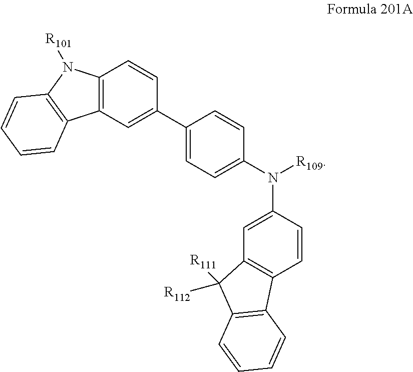

Detailed descriptions about R₁₀₁, R₁₁₁, R₁₁₂, and R₁₀₉ in Formula 201A are already described above.

For example, the compound represented by Formula 201 and the compound represented by Formula 202 may include compounds HT1 to HT20 illustrated below, but are not limited thereto.

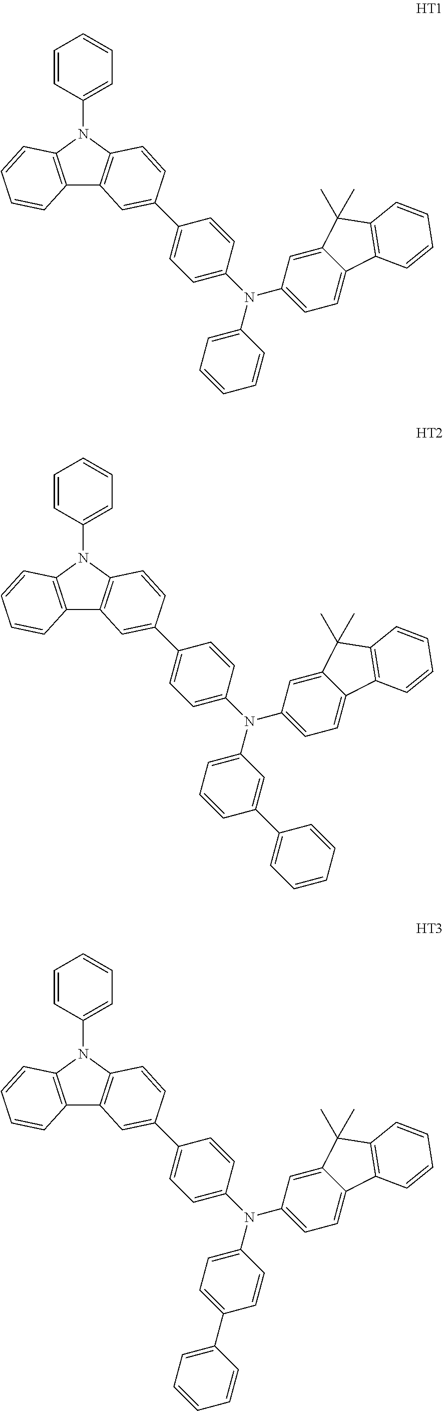 [SMILES: CC1(C)c2ccccc2-c2ccc(N(c3ccccc3)c3ccc(-c4ccc5c(c4)c4ccccc4n5-c4ccccc4)cc3)cc21 ; CC1(C)c2ccccc2-c2ccc(N(c3ccc(-c4ccc5c(c4)c4ccccc4n5-c4ccccc4)cc3)c3cccc(-c4ccccc4)c3)cc21 ; CC1(C)c2ccccc2-c2ccc(N(c3ccc(-c4ccccc4)cc3)c3ccc(-c4ccc5c(c4)c4ccccc4n5-c4ccccc4)cc3)cc21]

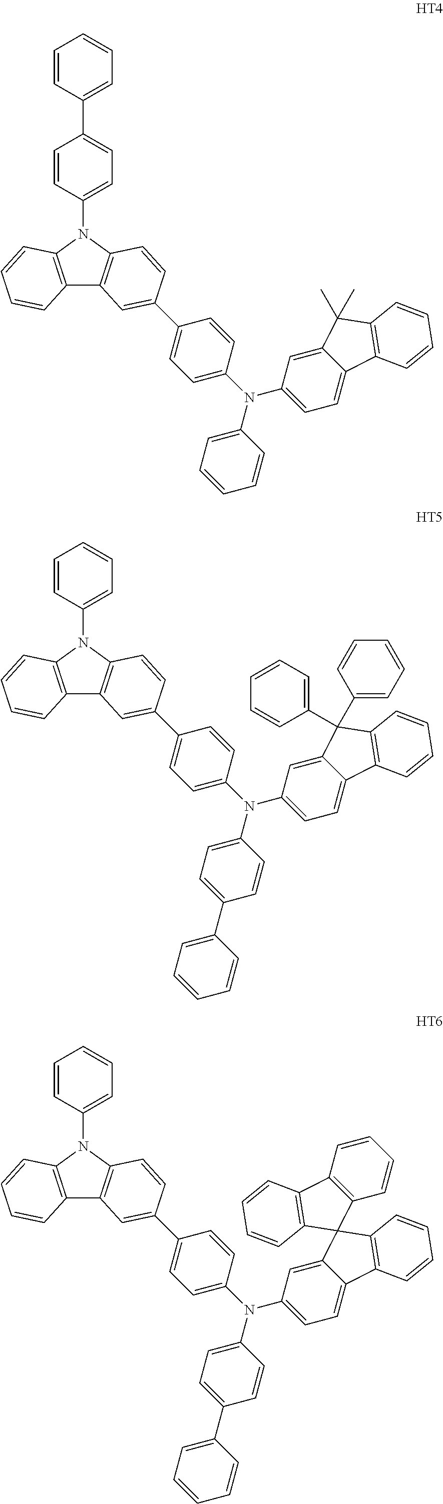 [SMILES: CC1(C)c2ccccc2-c2ccc(N(c3ccccc3)c3ccc(-c4ccc5c(c4)c4ccccc4n5-c4ccc(-c5ccccc5)cc4)cc3)cc21 ; c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1 ; c1ccc(-c2ccc(N(c3ccc(-c4ccc5c(c4)c4ccccc4n5-c4ccccc4)cc3)c3ccc4c(c3)C3(c5ccccc5-c5ccccc53)c3ccccc3-4)cc2)cc1]

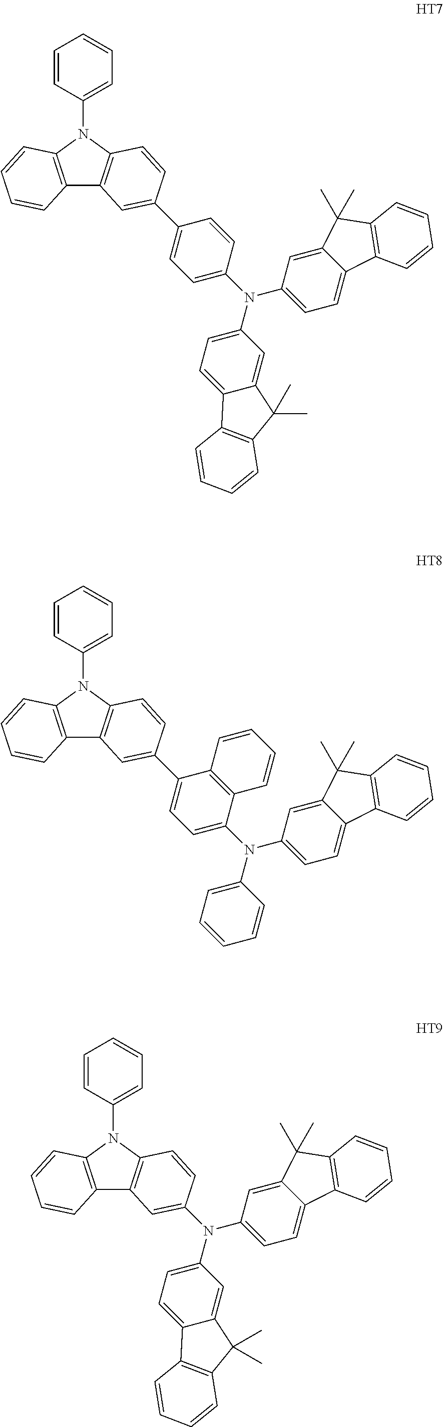 [SMILES: CC1(C)c2ccccc2-c2ccc(N(c3ccc(-c4ccc5c(c4)c4ccccc4n5-c4ccccc4)cc3)c3ccc4c(c3)C(C)(C)c3ccccc3-4)cc21 ; CC1(C)c2ccccc2-c2ccc(N(c3ccccc3)c3ccc(-c4ccc5c(c4)c4ccccc4n5-c4ccccc4)c4ccccc34)cc21 ; CC1(C)c2ccccc2-c2ccc(N(c3ccc4c(c3)C(C)(C)c3ccccc3-4)c3ccc4c(c3)c3ccccc3n4-c3ccccc3)cc21]

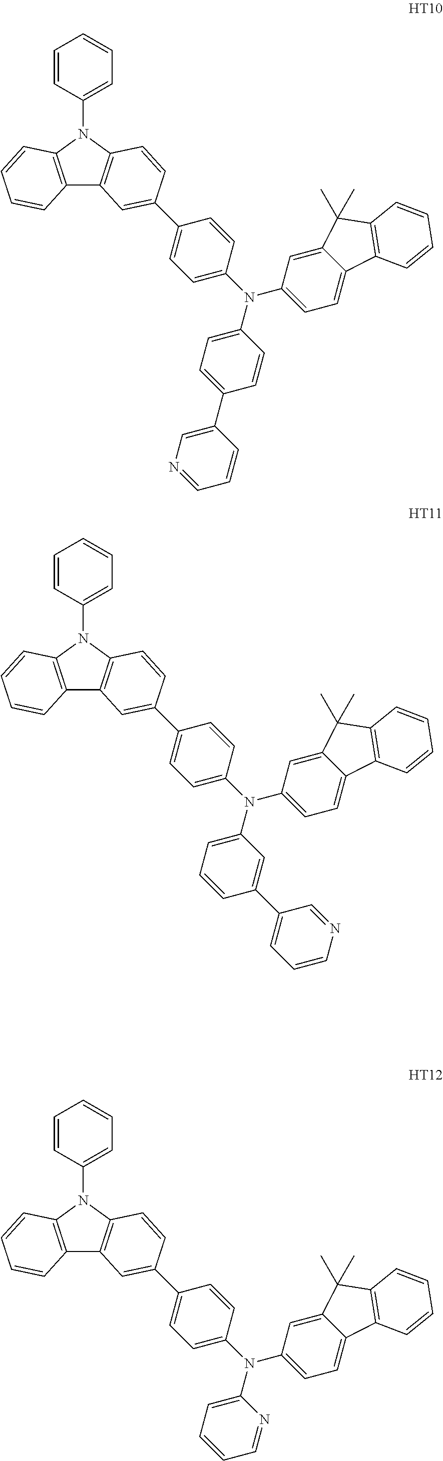 [SMILES: CC1(C)c2ccccc2-c2ccc(N(c3ccc(-c4cccnc4)cc3)c3ccc(-c4ccc5c(c4)c4ccccc4n5-c4ccccc4)cc3)cc21 ; CC1(C)c2ccccc2-c2ccc(N(c3ccc(-c4ccc5c(c4)c4ccccc4n5-c4ccccc4)cc3)c3cccc(-c4cccnc4)c3)cc21 ; CC1(C)c2ccccc2-c2ccc(N(c3ccc(-c4ccc5c(c4)c4ccccc4n5-c4ccccc4)cc3)c3ccccn3)cc21]

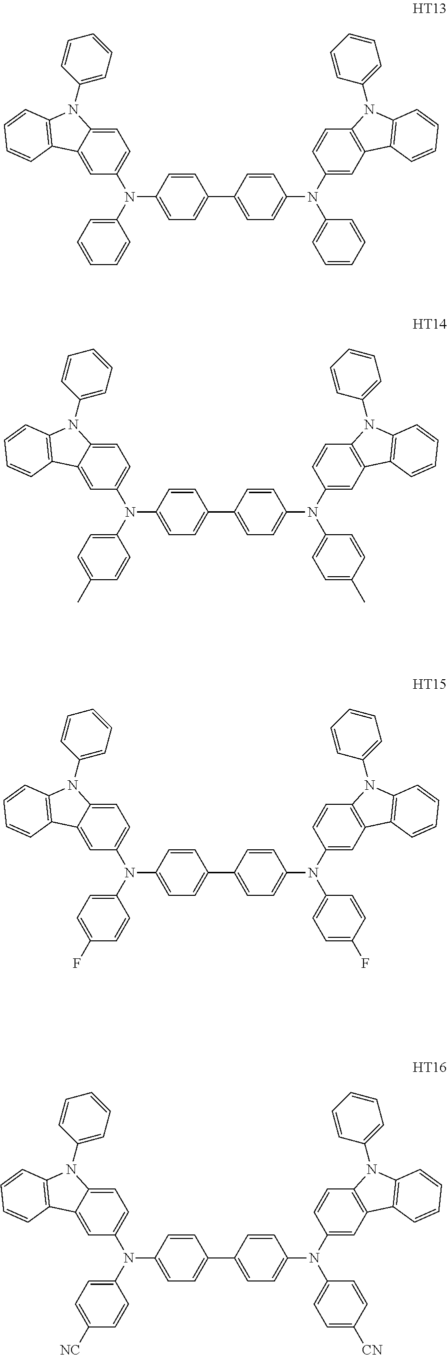 [SMILES: c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1 ; Cc1ccc(N(c2ccc(-c3ccc(N(c4ccc(C)cc4)c4ccc5c(c4)c4ccccc4n5-c4ccccc4)cc3)cc2)c2ccc3c(c2)c2ccccc2n3-c2ccccc2)cc1 ; Fc1ccc(N(c2ccc(-c3ccc(N(c4ccc(F)cc4)c4ccc5c(c4)c4ccccc4n5-c4ccccc4)cc3)cc2)c2ccc3c(c2)c2ccccc2n3-c2ccccc2)cc1 ; N#Cc1ccc(N(c2ccc(-c3ccc(N(c4ccc(C#N)cc4)c4ccc5c(c4)c4ccccc4n5-c4ccccc4)cc3)cc2)c2ccc3c(c2)c2ccccc2n3-c2ccccc2)cc1]

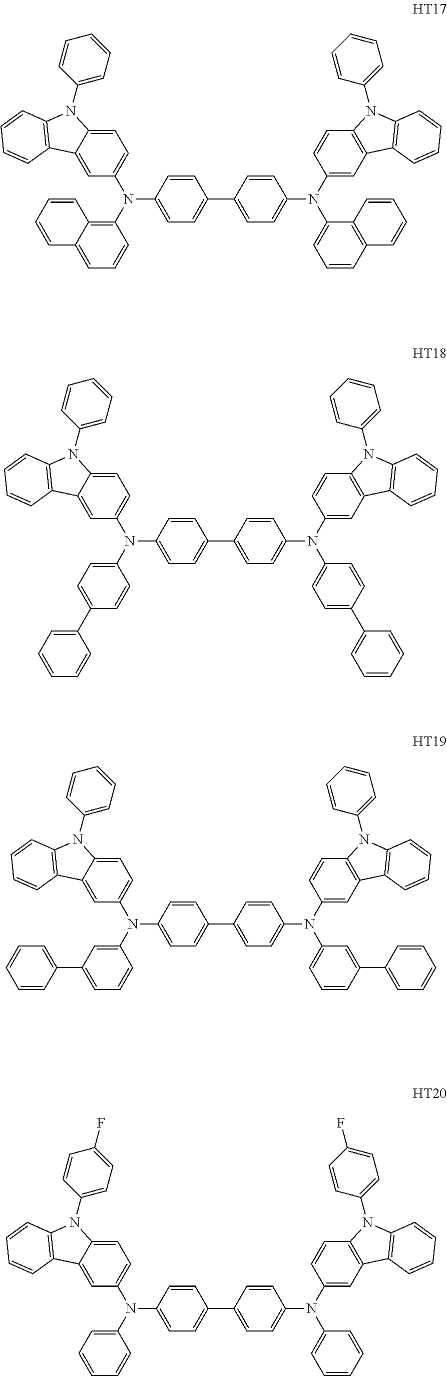 [SMILES: c1ccc(-n2c3ccccc3c3cc(N(c4ccc(-c5ccc(N(c6ccc7c(c6)c6ccccc6n7-c6ccccc6)c6cccc7ccccc67)cc5)cc4)c4cccc5ccccc45)ccc32)cc1 ; c1ccc(-c2ccc(N(c3ccc(-c4ccc(N(c5ccc(-c6ccccc6)cc5)c5ccc6c(c5)c5ccccc5n6-c5ccccc5)cc4)cc3)c3ccc4c(c3)c3ccccc3n4-c3ccccc3)cc2)cc1 ; c1ccc(-c2cccc(N(c3ccc(-c4ccc(N(c5cccc(-c6ccccc6)c5)c5ccc6c(c5)c5ccccc5n6-c5ccccc5)cc4)cc3)c3ccc4c(c3)c3ccccc3n4-c3ccccc3)c2)cc1 ; Fc1ccc(-n2c3ccccc3c3cc(N(c4ccccc4)c4ccc(-c5ccc(N(c6ccccc6)c6ccc7c(c6)c6ccccc6n7-c6ccc(F)cc6)cc5)cc4)ccc32)cc1]

A thickness of the hole transport region may be in a range of about 100 Angstroms (Å) to about 10,000 Å, for example, about 100 Å to about 1,000 Å. When the hole transport region includes at least one selected from a hole injection layer and a hole transport layer, the thickness of the hole injection layer may be in a range of about 100 Å to about 10,000 Å, for example, about 100 Å to about 1,000 Å, and the thickness of the hole transport layer may be in a range of about 50 Å to about 2,000 Å, for example, about 100 Å to about 1,500 Å. While not wishing to be bound by theory, it is understood that when the thicknesses of the hole transport region, the hole injection layer and the hole transport layer are within these ranges, satisfactory hole transporting characteristics may be obtained without a substantial increase in driving voltage.

The hole transport region may further include, in addition to these materials, a charge-generation material for the improvement of conductive properties. The charge-generation material may be homogeneously or non-homogeneously dispersed in the hole transport region.

The charge-generation material may be, for example, a p-dopant. The p-dopant may be one selected from a quinone derivative, a metal oxide, and a cyano group-containing compound, but embodiments are not limited thereto. Non-limiting examples of the p-dopant are a quinone derivative, such as tetracyanoquinonedimethane (TCNQ) or 2,3,5,6-tetrafluoro-tetracyano-1,4-benzoquinonedimethane (F4-TCNQ); a metal oxide, such as a tungsten oxide or a molybdenum oxide; and a cyano group-containing compound, such as Compound HT-D1 or HP-1, but are not limited thereto.

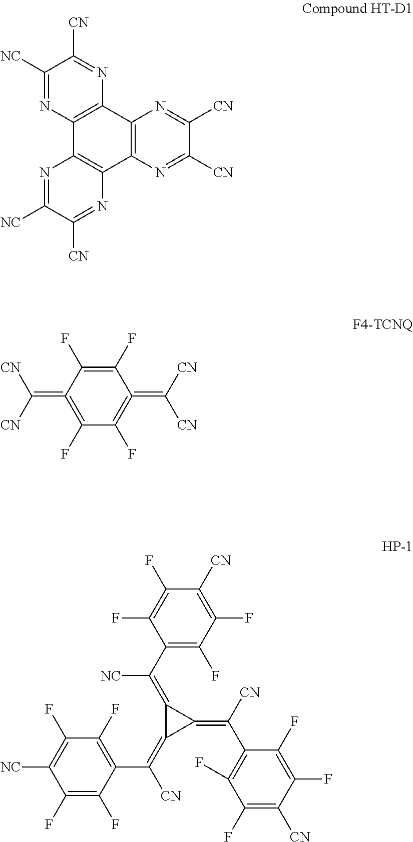

The hole transport region may include a buffer layer.

Also, the buffer layer may compensate for an optical resonance distance according to a wavelength of light emitted from the emission layer, and thus, efficiency of a formed organic light-emitting device may be improved.

Then, an emission layer may be formed on the hole transport region by vacuum deposition, spin coating, casting, LB deposition, or the like. When the emission layer is formed by vacuum deposition or spin coating, the deposition or coating conditions may be similar to those applied to form the hole injection layer although the deposition or coating conditions may vary depending on the material that is used to form the emission layer.

The electron transport region may further include an electron blocking layer.

The electron blocking layer may include, for example, mCP, but a material therefor is not limited thereto.

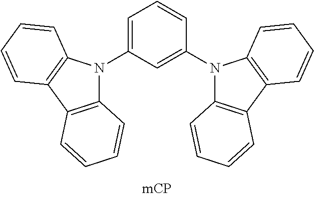 [SMILES: c1cc(-n2c3ccccc3c3ccccc32)cc(-n2c3ccccc3c3ccccc32)c1]

A thickness of the electron blocking layer may be in a range of about 50 Å to about 1,000 Å, for example, about 70 Å to about 500 Å. While not wishing to be bound by theory, it is understood that when the thickness of the electron blocking layer is within the range described above, the electron blocking layer may have satisfactory electron blocking characteristics without a substantial increase in driving voltage.

When the organic light-emitting device is a full color organic light-emitting device, the emission layer may be patterned into a red emission layer, a green emission layer, and a blue emission layer. In one or more embodiments, due to a stack structure including a red emission layer, a green emission layer, and/or a blue emission layer, the emission layer may emit white light.

The emission layer may include the condensed cyclic compound represented by Formula 1. For example, the emission layer may include the compound represented by Formula 1 alone. In one or more embodiments, the emission layer may include a host and a dopant, and the host may include the condensed cyclic compound represented by Formula 1. In one or more embodiments, the emission layer may include a host and a dopant, and the dopant may include the condensed cyclic compound represented by Formula 1

In one or more embodiments, the dopant in the emission layer may be a phosphorescent dopant, and the phosphorescent dopant may include an organometallic compound represented by Formula 81 below:

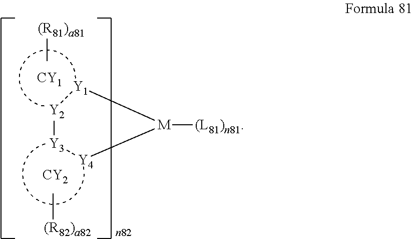

In Formula 81,

M may be selected from iridium (Ir), platinum (Pt), osmium (Os), titanium (Ti), zirconium (Zr), hafnium (Hf), europium (Eu), terbium (Tb), and thulium (Tm),

Y₁ to Y₄ may each independently be carbon(C) or nitrogen (N),

Y₁ and Y₂ may be linked via a single bond or a double bond, Y₃ and Y₄ may be linked via a single bond or a double bond,

CY₁ and CY₂ may each independently be selected from a benzene ring, a naphthalene ring, a fluorene ring, a spiro-fluorene ring, an indene ring, a pyrrole ring, a thiophene ring, a furan ring, an imidazole ring, a pyrazole ring, a thiazole ring, an isothiazole ring, an oxazole ring, an isoxazole ring, a pyridine ring, a pyrazine ring, a pyrimidine ring, a pyridazine ring, a quinoline ring, an isoquinoline ring, a benzoquinoline ring, a quinoxaline ring, a quinazoline ring, a carbazole ring, a benzimidazole ring, a benzofuran ring, a benzothiophene ring, an isobenzothiophene ring, a benzoxazole ring, an isobenzoxazole ring, a triazole ring, a tetrazole ring, an oxadiazole ring, a triazine ring, a dibenzofuran ring, or a dibenzothiophene ring, and CY₁ and CY₂ may optionally be linked via a single bond or an organic linking group,

R₈₁ and R₈₂ may each independently be selected from hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, —SF₅, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent non-aromatic condensed polycyclic group, a substituted or unsubstituted monovalent non-aromatic condensed heteropolycyclic group, —N(Q₁)(Q₂), —Si(Q₃)(Q₄)(Q₅), and —B(Q₆)(Q₇),

a81 and a82 may each independently be an integer selected from 1 to 5,

n81 may be an integer selected from 0 to 4,

n82 may be 1, 2, or 3, and

L₈₁ may be a monovalent organic ligand, a divalent organic ligand, or a trivalent organic ligand.

R₈₁ and R₈₂ are the same as described in connection with R₁₁.

The phosphorescent dopant may include at least one selected from FIr₆ and Compounds PD1 to PD78, but embodiments are not limited thereto:

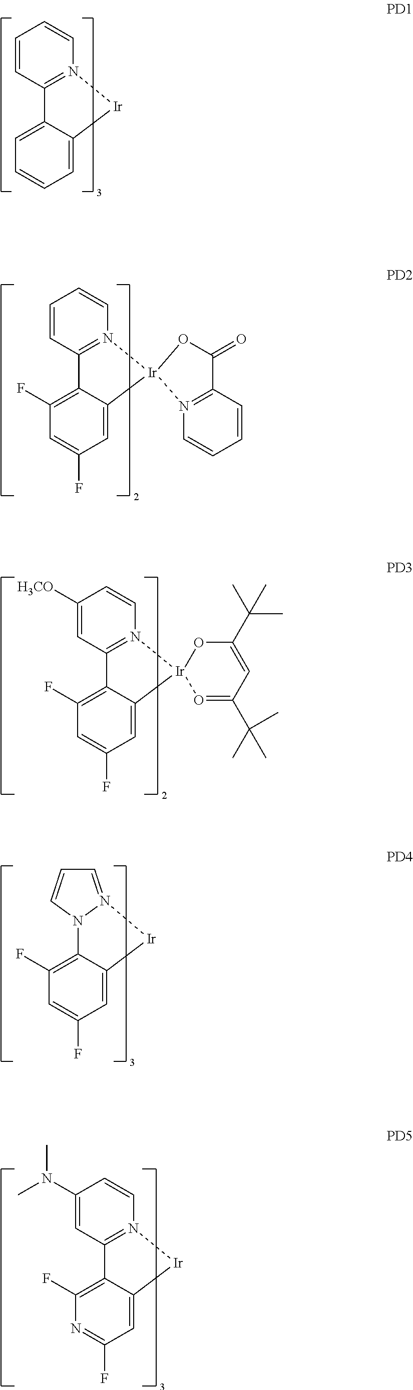

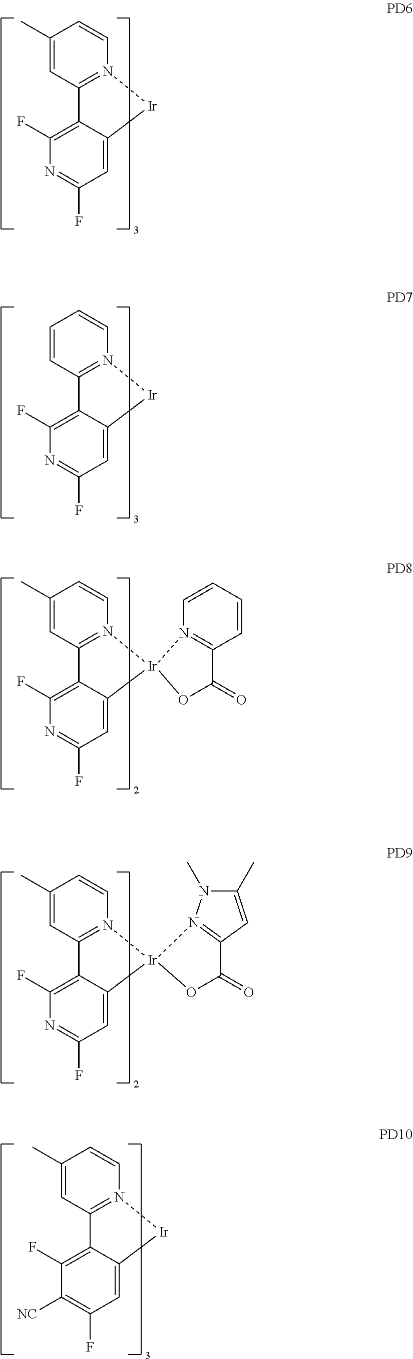

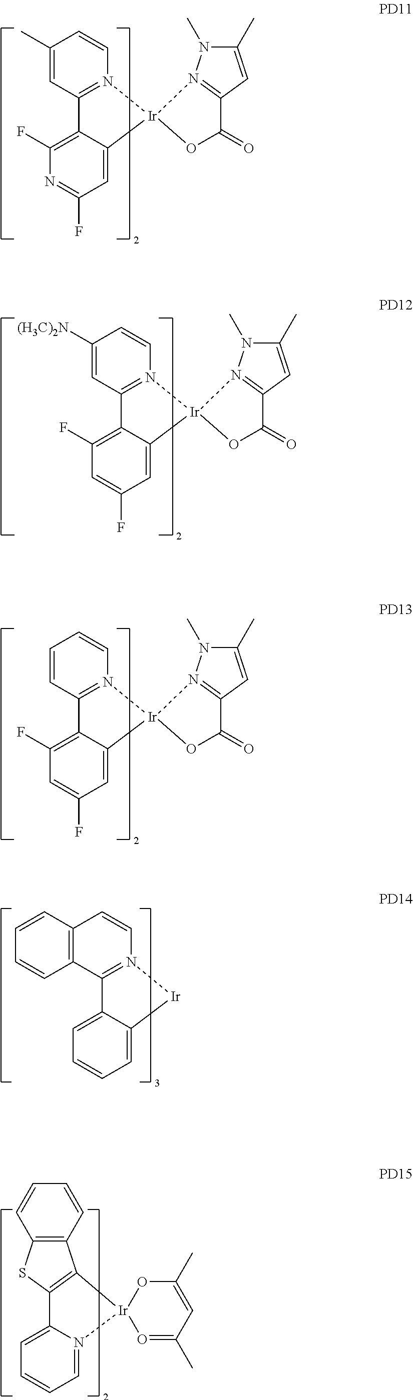

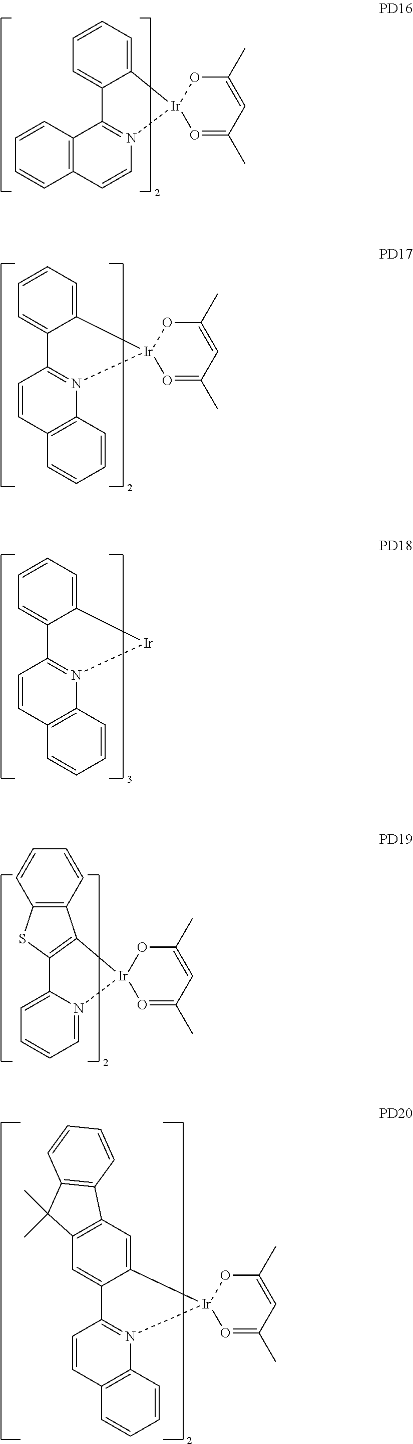

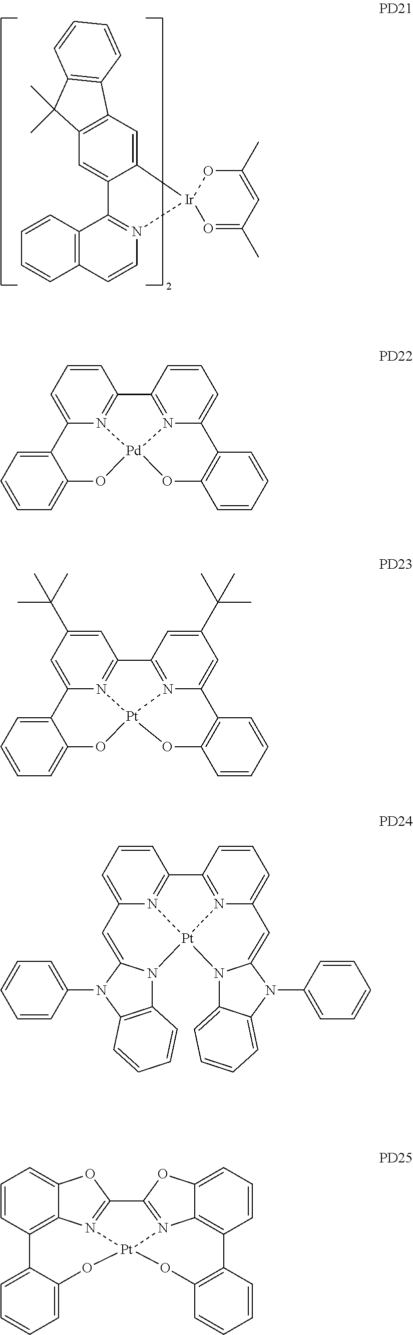

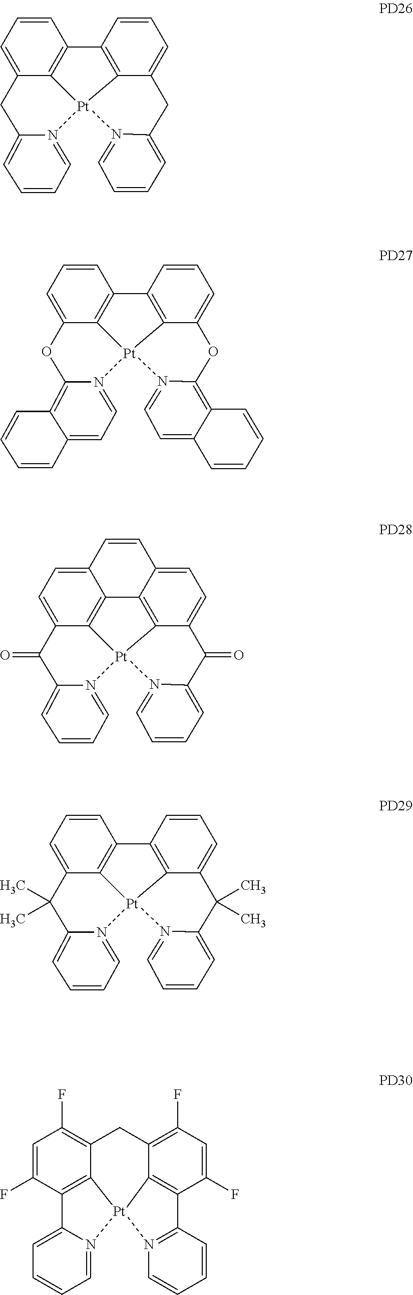

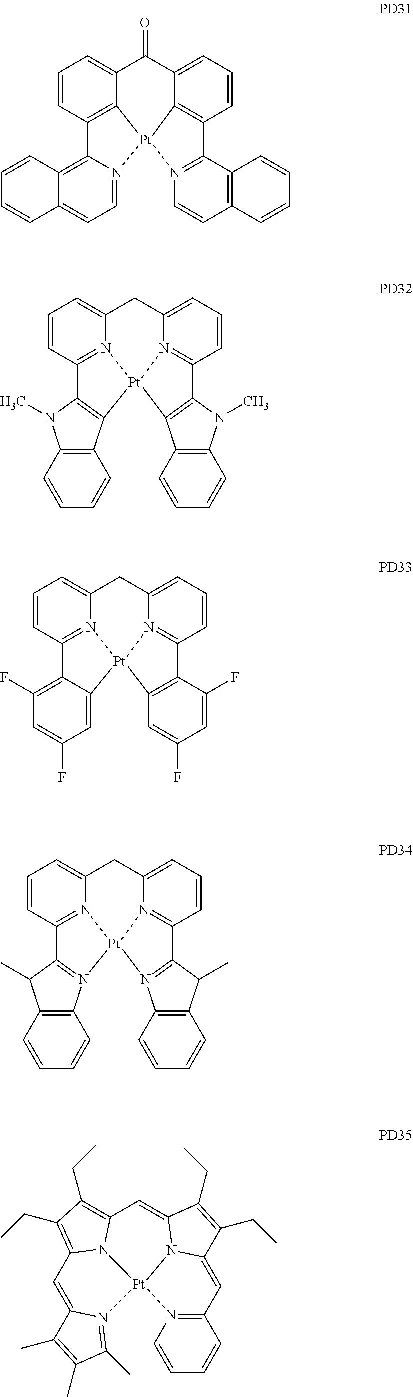

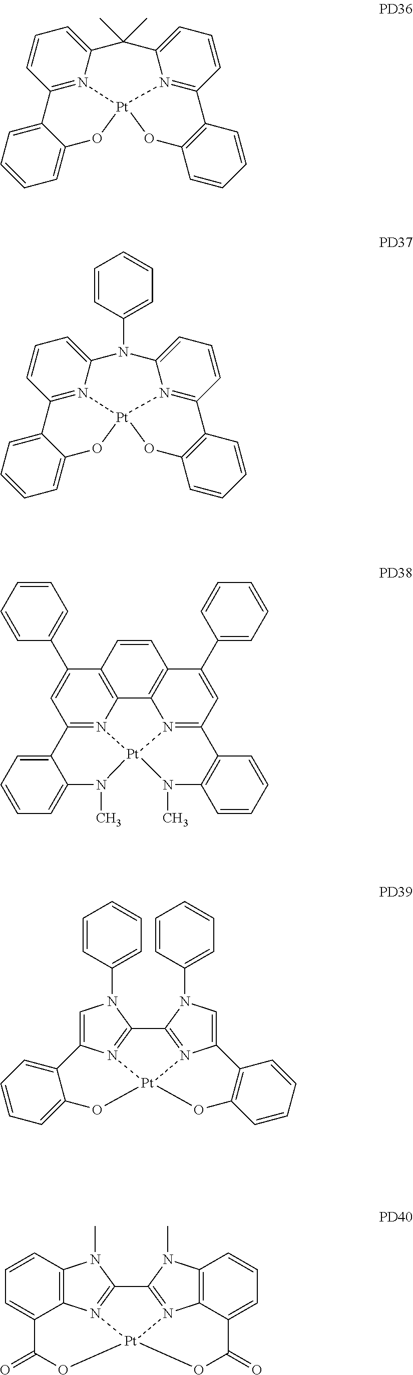

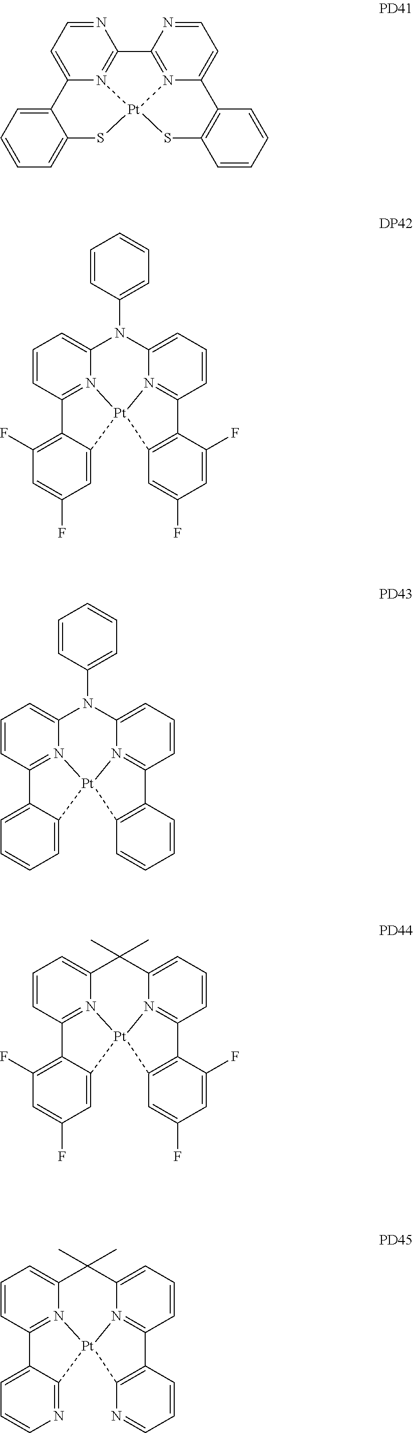

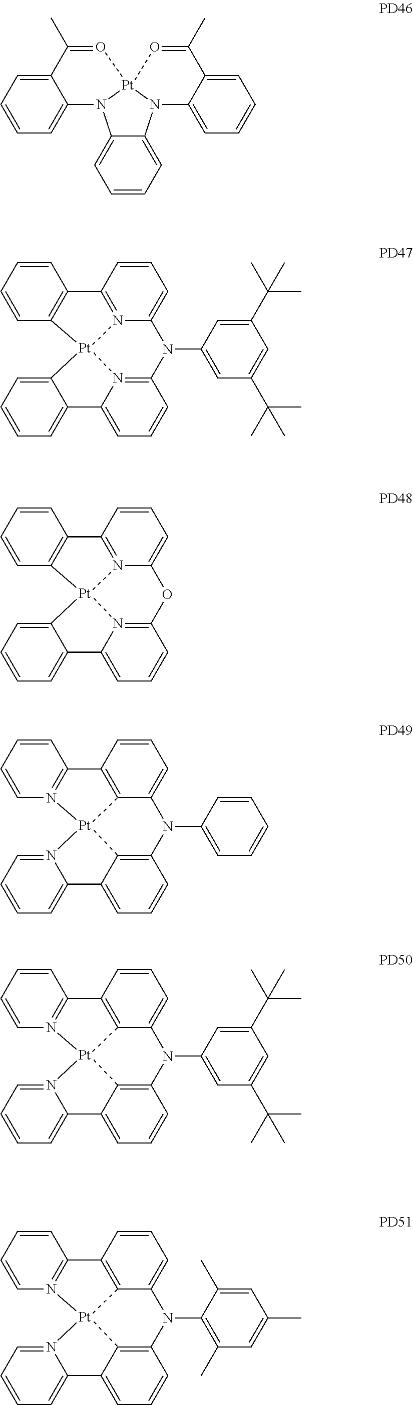

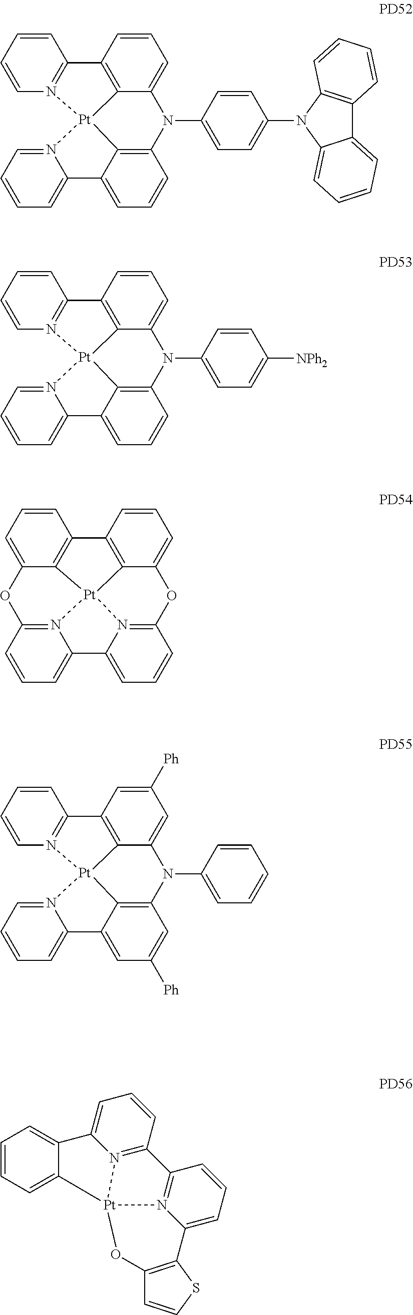

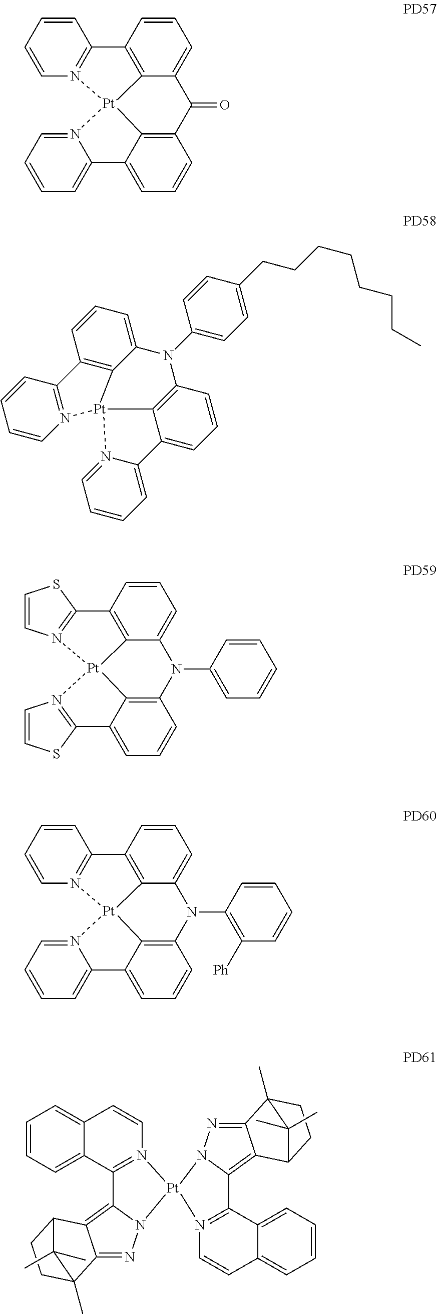

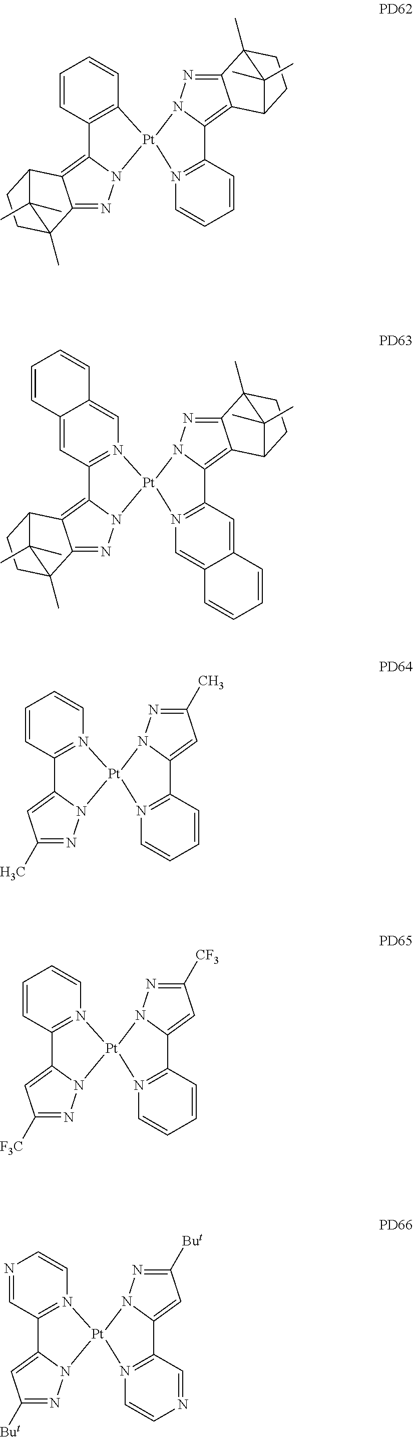

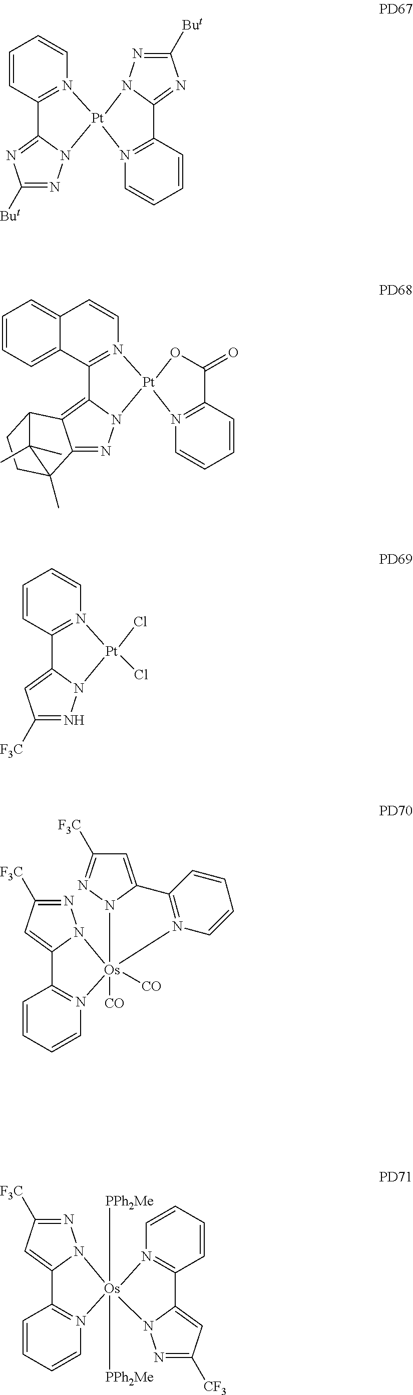

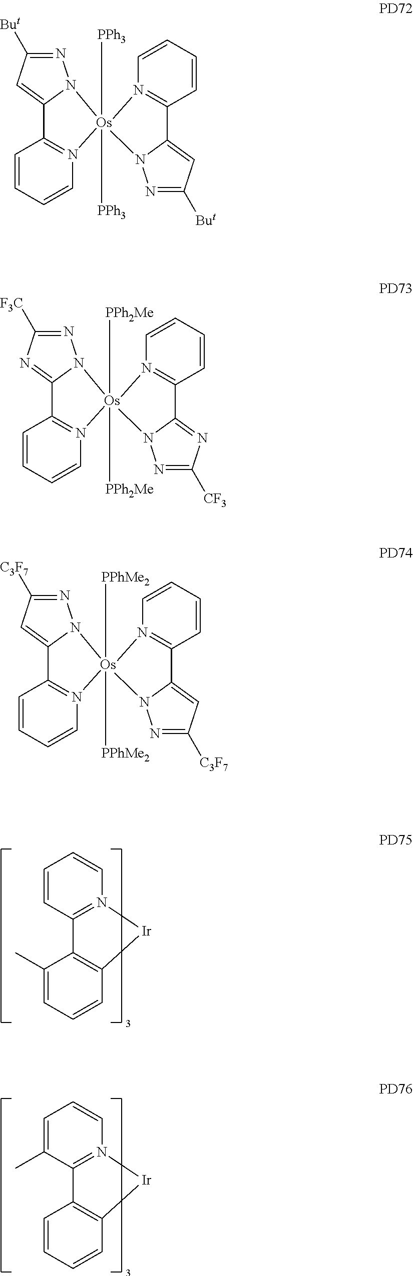

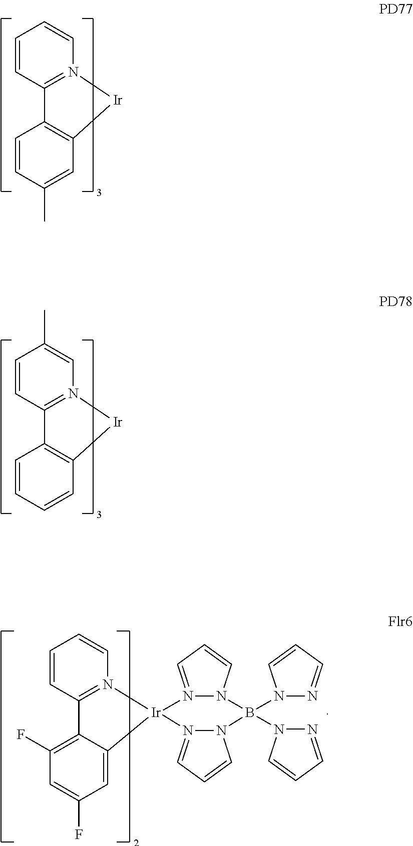

In one or more embodiments, the phosphorescent dopant may include PtOEP:

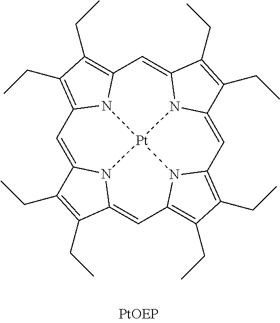

When the emission layer includes a host and a dopant, an amount of the dopant may be in a range of about 0.01 to about 20 parts by weight based on 100 parts by weight of the host, but is not limited thereto.

A thickness of the emission layer may be in a range of about 100 Å to about 1,000 Å, for example, about 200 Å to about 600 Å. While not wishing to be bound by theory, it is understood that when the thickness of the emission layer is within this range, excellent light emission characteristics may be obtained without a substantial increase in driving voltage.

Then, an electron transport region may be disposed on the emission layer.

The electron transport region may include at least one selected from a hole blocking layer, an electron transport layer, and an electron injection layer.

For example, the electron transport region may have a hole blocking layer/electron transport layer/electron injection layer structure or an electron transport layer/electron injection layer structure, but the structure of the electron transport region is not limited thereto. The electron transport layer may have a single-layered structure or a multi-layered structure including two or more different materials.

Conditions for forming the hole blocking layer, the electron transport layer, and the electron injection layer which constitute the electron transport region may be understood by referring to the conditions for forming the hole injection layer.

When the electron transport region includes a hole blocking layer, the hole blocking layer may include, for example, at least one of BCP and Bphen, but may also include other materials.

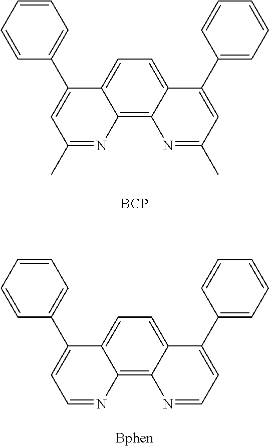 [SMILES: Cc1cc(-c2ccccc2)c2ccc3c(-c4ccccc4)cc(C)nc3c2n1 ; "BCP" ; c1ccc(-c2ccnc3c2ccc2c(-c4ccccc4)ccnc23)cc1]

The hole blocking layer may include the condensed cyclic compound represented by Formula 1.

A thickness of the hole blocking layer may be in a range of about 20 Å to about 1,000 Å, for example, about 30 Å to about 300 Å. While not wishing to be bound by theory, it is understood that when the thickness of the hole blocking layer is within these ranges, the hole blocking layer may have excellent hole blocking characteristics without a substantial increase in driving voltage.

The electron transport layer may further include at least one selected from BCP, Bphen, Alq₃, BAIq, TAZ, and NTAZ.

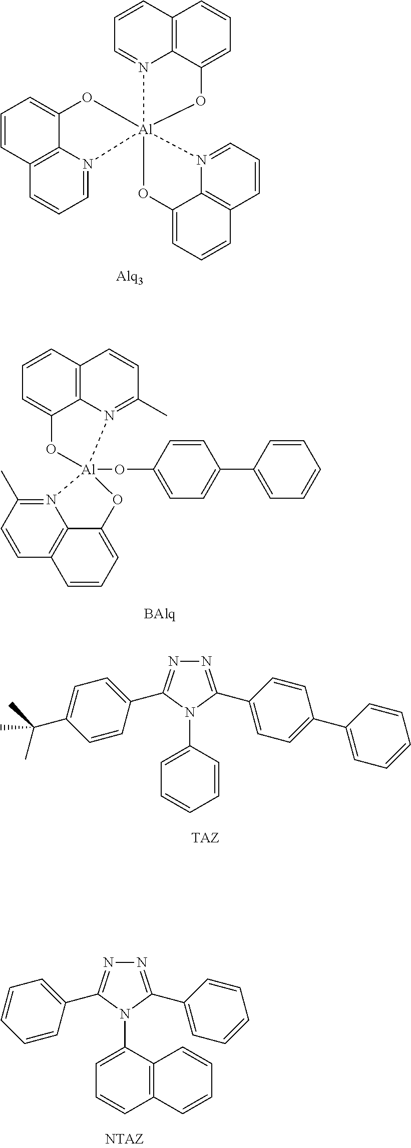

In one or more embodiments, the electron transport layer may include at least one selected from Compounds ET1, ET2, and ET3, but embodiments are not limited thereto:

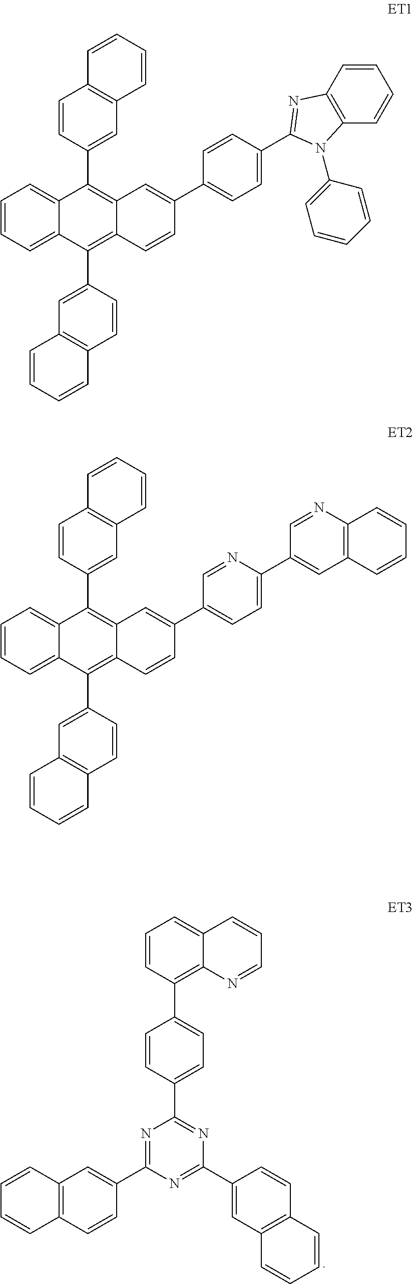 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc2cc(-c3c4ccccc4c(-c4ccc5ccccc5c4)c4cc(-c5ccc(-c6cnc7ccccc7c6)nc5)ccc34)ccc2c1 ; c1ccc2cc(-c3nc(-c4ccc(-c5cccc6cccnc56)cc4)nc(-c4ccc5ccccc5c4)n3)ccc2c1]

A thickness of the electron transport layer may be in a range of about 100 Å to about 1,000 Å, for example, about 150 Å to about 500 Å. While not wishing to be bound by theory, it is understood that when the thickness of the electron transport layer is within the range described above, the electron transport layer may have satisfactory electron transport characteristics without a substantial increase in driving voltage.

Also, the electron transport layer may further include, in addition to the materials described above, a metal-containing material.

The metal-containing material may include a Li complex. The Li complex may include, for example, Compound ET-D1 (lithium quinolate, LiQ) or ET-D2.

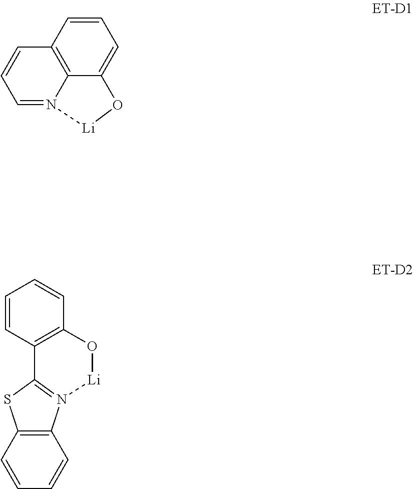

The electron transport region may include an electron injection layer that promotes flow of electrons from the second electrode 19 thereinto.

The electron injection layer may include at least one selected from LiQ, LiF, NaCl, CsF, Li₂O, and BaO.

A thickness of the electron injection layer may be in a range of about 1 Å to about 100 Å, for example, about 3 Å to about 90 Å. While not wishing to be bound by theory, it is understood that when the thickness of the electron injection layer is within the range described above, the electron injection layer may have satisfactory electron injection characteristics without a substantial increase in driving voltage.

The second electrode 19 is disposed on the organic layer 15. The second electrode 19 may be a cathode. A material for forming the second electrode 19 may be metal, an alloy, an electrically conductive compound, or a combination thereof, which have a relatively low work function. For example, lithium (Li), magnesium (Mg), aluminum (Al), aluminum-lithium (Al—Li), calcium (Ca), magnesium-indium (Mg—In), or magnesium-silver (Mg—Ag) may be formed as the material for forming the second electrode 19. To manufacture a top-emission type light-emitting device, a transmissive electrode formed using ITO or IZO may be used as the second electrode 19.

Hereinbefore, the organic light-emitting device has been described with reference to FIG. 1, but is not limited thereto.

The term “C₁-C₆₀ alkyl group” as used herein refers to a linear or branched aliphatic saturated hydrocarbon monovalent group having 1 to 60 carbon atoms. Non-limiting examples thereof include a methyl group, an ethyl group, a propyl group, an iso-butyl group, a sec-butyl group, a tert-butyl group, a pentyl group, an iso-amyl group, and a hexyl group. The term “C₁-C₆₀ alkylene group” as used herein refers to a divalent group having the same structure as the C₁-C₆₀ alkyl group.

The term “C₁-C₆₀ alkoxy group” as used herein refers to a monovalent group represented by —OA₁₀₁ (wherein A₁₀₁ is the C₁-C₆₀ alkyl group). Non-limiting examples thereof include a methoxy group, an ethoxy group, and an iso-propyloxy (iso-propoxy) group.

The term “C₂-C₆₀ alkenyl group” as used herein refers to a hydrocarbon group having at least one carbon-carbon double bond in the middle or at the terminus of the C₂-C₆₀ alkyl group. Non-limiting examples thereof include an ethenyl group, a propenyl group, and a butenyl group. The term “C₂-C₆₀ alkenylene group” as used herein refers to a divalent group having the same structure as the C₂-C₆₀ alkenyl group.

The term “C₂-C₆₀ alkynyl group” as used herein refers to a hydrocarbon group having at least one carbon-carbon triple bond in the middle or at the terminus of the C₂-C₆₀ alkyl group. Non-limiting examples thereof include an ethynyl group and a propynyl group. The term “C₂-C₆₀ alkynylene group” as used herein refers to a divalent group having the same structure as the C₂-C₆₀ alkynyl group.

The term “C₃-C₁₀ cycloalkyl group” as used herein refers to a monovalent saturated hydrocarbon monocyclic group having 3 to 10 carbon atoms. Non-limiting examples thereof include a cyclopropyl group, a cyclobutyl group, a cyclopentyl group, a cyclohexyl group, and a cycloheptyl group. The term “C₃-C₁₀ cycloalkylene group” as used herein refers to a divalent group having the same structure as the C₃-C₁₀ cycloalkyl group.

The term “C₁-C₁₀ heterocycloalkyl group” as used herein refers to a monovalent saturated monocyclic group having at least one heteroatom selected from N, O, P, Si and S as a ring-forming atom, and 1 to 10 carbon atoms. Non-limiting examples thereof include a tetrahydrofuranyl group and a tetrahydrothiophenyl group. The term “C₁-C₁₀ heterocycloalkylene group” as used herein refers to a divalent group having the same structure as the C₁-C₁₀ heterocycloalkyl group.

The term “C₃-C₁₀ cycloalkenyl group” as used herein refers to a monovalent monocyclic group that has 3 to 10 carbon atoms and at least one carbon-carbon double bond in the ring thereof, and which is not aromatic. Examples thereof include a cyclopentenyl group, a cyclohexenyl group, and a cycloheptenyl group. The term “C₃-C₁₀ cycloalkenylene group” as used herein refers to a divalent group having the same structure as the C₃-C₁₀ cycloalkenyl group.

The term “C₁-C₁₀ heterocycloalkenyl group” as used herein refers to a monovalent monocyclic group that has at least one heteroatom selected from N, O, P, Si, and S as a ring-forming atom, 1 to 10 carbon atoms, and at least one carbon-carbon double bond in its ring. Non-limiting examples of the C₂-C₁₀ heterocycloalkenyl group include a 2,3-dihydrofuranyl group and a 2,3-dihydrothiophenyl group. The term “C₁-C₁₀ heterocycloalkenylene group” as used herein refers to a divalent group having the same structure as the C₁-C₁₀ heterocycloalkenyl group.

The term “C₆-C₆₀ aryl group” as used herein refers to a monovalent group having a carbocyclic aromatic system having 6 to 60 carbon atoms, and the term “C₆-C₆₀ arylene group” as used herein refers to a divalent group having a carbocyclic aromatic system having 6 to 60 carbon atoms. Non-limiting examples of the C₆-C₆₀ aryl group include a phenyl group, a naphthyl group, an anthracenyl group, a phenanthrenyl group, a pyrenyl group, and a chrysenyl group. When the C₆-C₆₀ aryl group and the C₆-C₆₀ arylene group each include two or more rings, the rings may be fused to each other.

The term “C₁-C₆₀ heteroaryl group” as used herein refers to a monovalent group having a heterocyclic aromatic system that has at least one heteroatom selected from N, O, P, and S as a ring-forming atom, and 1 to 60 carbon atoms. The term C₁-C₆₀ heteroarylene group” as used herein refers to a divalent group having a heterocyclic aromatic system that has at least one heteroatom selected from N, O, P, and S as a ring-forming atom, and 1 to 60 carbon atoms. Non-limiting examples of the C₁-C₆₀ heteroaryl group include a pyridinyl group, a pyrimidinyl group, a pyrazinyl group, a pyridazinyl group, a triazinyl group, a quinolinyl group, and an isoquinolinyl group. When the C₁-C₆₀ heteroaryl group and the C₁-C₆₀ heteroarylene group each include two or more rings, the rings may be fused to each other.

The term “C₆-C₆₀ aryloxy group” as used herein refers to —OA₁₀₂ (wherein A₁₀₂ is the C₆-C₆₀ aryl group), and a C₆-C₆₀ arylthio group used herein indicates —SA₁₀₃ (wherein A₁₀₃ is the C₆-C₆₀ aryl group).

The term “monovalent non-aromatic condensed polycyclic group” as used herein refers to a monovalent group that has two or more rings condensed to each other, which includes only carbon atoms (for example, the number of carbon atoms may be in a range of 8 to 60) as ring-forming atoms, and which is non-aromatic in the entire molecular structure. A non-limiting example of the monovalent non-aromatic condensed polycyclic group is a fluorenyl group. The term “divalent non-aromatic condensed polycyclic group,” as used herein, refers to a divalent group having the same structure as the monovalent non-aromatic condensed polycyclic group.

The term “monovalent non-aromatic condensed heteropolycyclic group” as used herein refers to a monovalent group that has two or more rings condensed to each other, that has a heteroatom selected from N, O, P, Si, and S, other than carbon atoms (for example, the number of carbon atoms may be in a range of 2 to 60), as a ring-forming atom, and that is non-aromaticity in the entire molecular structure. Non-limiting examples of the monovalent non-aromatic condensed heteropolycyclic group include a carbazolyl group. The term “divalent non-aromatic condensed heteropolycyclic group,” as used herein, refers to a divalent group having the same structure as the monovalent non-aromatic condensed heteropolycyclic group.

At least one substituent selected from a substituent(s) of the substituted C₁-C₆₀ alkyl group, the substituted C₂-C₆₀ alkenyl group, the substituted C₂-C₆₀ alkynyl group, the substituted C₁-C₆₀ alkoxy group, the substituted C₃-C₁₀ cycloalkyl group, the substituted C₁-C₁₀ heterocycloalkyl group, the substituted C₃-C₁₀ cycloalkenyl group, the substituted C₁-C₁₀ heterocycloalkenyl group, the substituted C₆-C₆₀ aryl group, the substituted C₆-C₆₀ aryloxy group, the substituted C₆-C₆₀ arylthio group, the substituted C₁-C₆₀ heteroaryl group, the substituted monovalent non-aromatic condensed polycyclic group, and the substituted monovalent non-aromatic condensed heteropolycyclic group in Formula 1 may be selected from deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, and —Si(Q₃₁)(Q₃₂)(Q₃₃),

wherein Q₁ to Q₃, Q₁₁ to Q₁₃, Q₂₁ to Q₂₃, and Q₃₁ to Q₃₃ may each independently be selected from hydrogen, a C₁-C₆₀ alkyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₁-C₆₀ heteroaryl group, a monovalent non-aromatic condensed polycyclic group, and a monovalent non-aromatic condensed heteropolycyclic group.

When a group containing a specified number of carbon atoms is substituted with any of the groups listed in the preceding paragraph, the number of carbon atoms in the resulting “substituted” group is defined as the sum of the carbon atoms contained in the original (unsubstituted) group and the carbon atoms (if any) contained in the substituent. For example, when the term “substituted C₁-C₃₀ alkyl” refers to a C₁-C₃₀ alkyl group substituted with C₆-C₃₀ aryl group, the total number of carbon atoms in the resulting aryl substituted alkyl group is C₇-C₆₀.

The expressions * and *′ used herein each refer to a binding site to a neighboring atom in a corresponding Formula.

Hereinafter, a compound and an organic light-emitting device according to embodiments are described in detail with reference to Synthesis Example and Examples. However, the organic light-emitting device is not limited thereto. The wording “‘B’ was used instead of ‘A’” used in describing Synthesis Examples means that a molar equivalent of ‘A’ was identical to a molar equivalent of ‘B’.

### EXAMPLES

### Synthesis Example 1: Synthesis of Compound 27

### (1) Synthesis of Intermediate 1

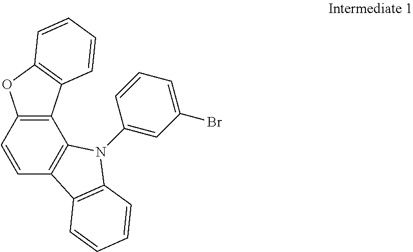 [SMILES: Brc1cccc(-n2c3ccccc3c3ccc4oc5ccccc5c4c32)c1]

3 grams (g) (73.46 millimoles, mmol) of NaH (60% in mineral oil) was added into a well-dried 500-ml round-bottom flask (RBF) and 200 milliliters (ml) of dimethylformamide (DMF) was added thereto. The mixture was stirred at a temperature of 0° C. under a nitrogen atmosphere. 18 g (69.96 mmol) of 12H-benzofuro[3,2-a]carbazole was slowly dissolved in 100 ml of DMF and added to the mixture. The resultant was heated to room temperature and stirred for 3 hours. 19.6 g (112 mmol) of 1-bromo-3-fluorobenzene was slowly added to the reaction mixture. The reaction temperature was raised to 150° C., and the resultant substance was stirred for 22 hours. A solid product was extracted by using methylene chloride (MC) and was dried by using MgSO₄. The solvent was concentrated under reduced pressure. A solution dissolved in hot toluene was filtered through a thin plug of silica, and the filtrate was concentrated under reduced pressure. A precipitate was formed upon treatment with MC/methanol mixture and the resultant was passed through a filtering paper. The obtained solid was dried in a vacuum oven to obtain 21 g (73%) of Intermediate 1.

### (2) Synthesis of Compound 27

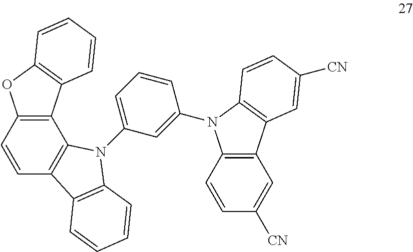 [SMILES: N#Cc1ccc2c(c1)c1cc(C#N)ccc1n2-c1cccc(-n2c3ccccc3c3ccc4oc5ccccc5c4c32)c1]

15 g (35 mmol) of Intermediate 1, 7.2 g (33 mmol) of 9H-carbazole-3,6-dicarbonitrile, 3.2 g (16.5 mmol) of CuI, 9.1 g (66 mmol) of K₂CO₃, and 6 g (33 mmol) of 1,10-phenanthroline were added into a 250-ml RBF, and 150 ml of DMF was added thereto. The mixture was stirred at a temperature of 165° C. for 24 hours. The reaction mixture was cooled and poured into a mixed solution of water and methanol to obtain a precipitate. The obtained precipitate was passed through a filtering paper. The obtained solid was washed by methanol and was dried in a vacuum oven. The solid was dissolved in hot MC and passed through a thin plug of silica, and the filtrate was concentrated. The recrystallization was performed through ethyl acetate to obtain 10.5 g (58%) of Compound 27.

MALDI-TOF Mass (calculated value: 548.59 g/mol, measured value: 548.28 g/mol)

### Synthesis Example 2: Synthesis of Compound 29

### (1) Synthesis of Intermediate 2

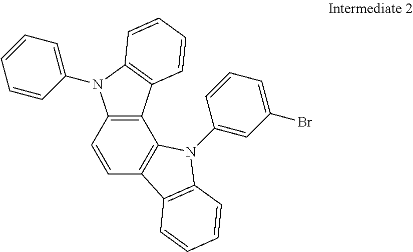 [SMILES: Brc1cccc(-n2c3ccccc3c3ccc4c(c5ccccc5n4-c4ccccc4)c32)c1]

15 g (45 mmol) of 5-phenyl-5,12-dihydroindolo[3,2-a]carbazole, 15.3 g (54 mmol) of 3-bromoiodobenzene, 4.3 g (22.6 mmol) of CuI, 12.5 g (90 mmol) of K₂CO₃, and 9.7 g (54 mmol) of 1,10-phenanthroline were added into a 250-ml RBF, and 180 ml of DMF was added thereto. The mixture was stirred at a temperature of 165° C. for 24 hours. The reaction mixture was cooled and washed by water, and a solid product was extracted by MC. The solid product was dried by MgSO₄ and distilled under reduced pressure. 12.8 g (58%) of Intermediate 2 was obtained after performing silica column chromatography (MC:Hexane=1:4).

### (2) Synthesis of Compound 29

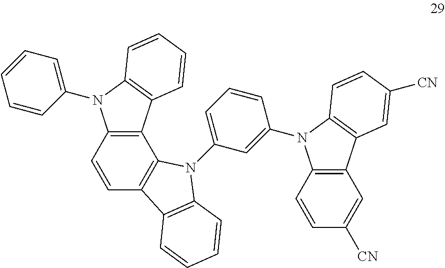 [SMILES: N#Cc1ccc2c(c1)c1cc(C#N)ccc1n2-c1cccc(-n2c3ccccc3c3ccc4c(c5ccccc5n4-c4ccccc4)c32)c1]

Compound 29 (42%) was synthesized in the same manner as in Synthesis of Compound 27 of Synthesis Example 1, except that Intermediate 2 was used instead of Intermediate 1 in synthesizing Compound 20.

MALDI-TOF Mass (calculated value: 623.70 g/mol, measured value: 623.42 g/mol)

### Synthesis Example 3: Synthesis of Compound 33

### (1) Synthesis of Intermediate 3

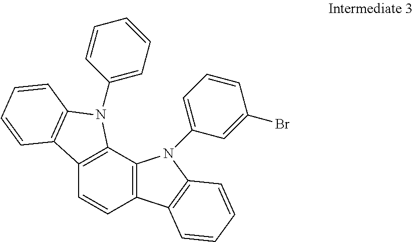 [SMILES: Brc1cccc(-n2c3ccccc3c3ccc4c5ccccc5n(-c5ccccc5)c4c32)c1]

Intermediate 3 (22%) was synthesized in the same manner as in Synthesis of Intermediate 2 of Synthesis Example 2, except that 11-phenyl-11,12-dihydroindolo[2,3-a]carbazole was used instead of 5-phenyl-5,12-dihydroindolo[3,2-a]carbazole in synthesizing Compound 3.

### (2) Synthesis of Compound 33

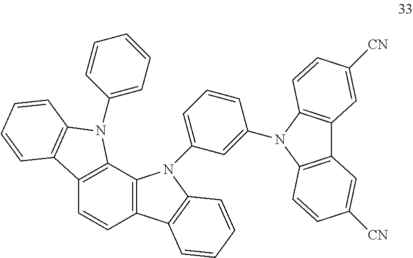 [SMILES: N#Cc1ccc2c(c1)c1cc(C#N)ccc1n2-c1cccc(-n2c3ccccc3c3ccc4c5ccccc5n(-c5ccccc5)c4c32)c1]

Compound 33 (47%) was synthesized in the same manner as in Syntheses of Compound 27 of Synthesis Example 1, expect that Intermediate 3 was used instead of Intermediate 1 in synthesizing Compound 33.

MALDI-TOF Mass (calculated value: 623.70 g/mol, measured value: 623.41 g/mol)

### Synthesis Example 4: Synthesis of Compound 43

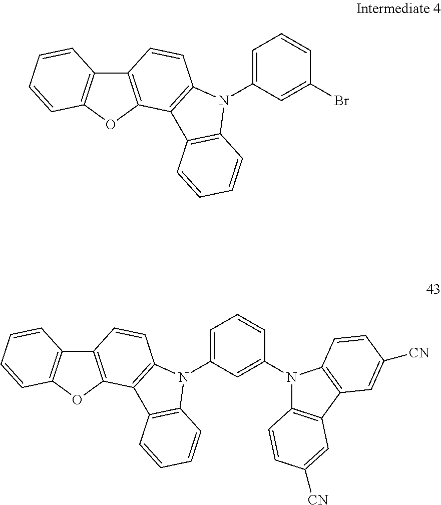 [SMILES: Brc1cccc(-n2c3ccccc3c3c4oc5ccccc5c4ccc32)c1 ; N#Cc1ccc2c(c1)c1cc(C#N)ccc1n2-c1cccc(-n2c3ccccc3c3c4oc5ccccc5c4ccc32)c1]

### (1) Synthesis of Intermediate 4

Intermediate 4 (81%) was synthesized in the same manner as Synthesis of Intermediate 1 of Synthesis Example 1, except that 5H-benzofuro[3,2-c]carbazole was used instead of 12H-benzofuro[3,2-a]carbazole in synthesizing Intermediate 4.

### (2) Synthesis of Compound 43

Compound 43 (60%) was synthesized in the same manner as in Synthesis of Compound 27 of Synthesis Example 1, except that Intermediate 4 was used instead of Intermediate 1 in synthesizing Compound 43.

MALDI-TOF Mass (calculated value: 548.59 g/mol, measured value: 548.28 g/mol)

### Synthesis Example 5: Synthesis of Compound 44

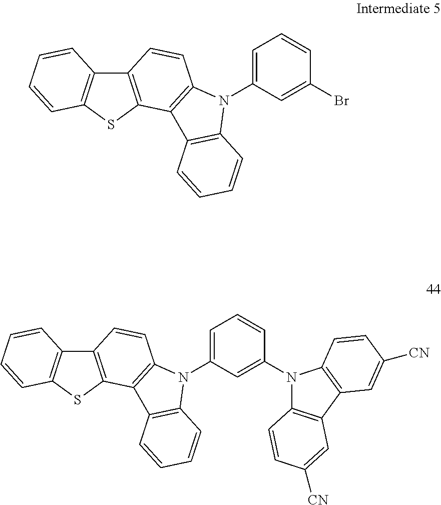 [SMILES: Brc1cccc(-n2c3ccccc3c3c4sc5ccccc5c4ccc32)c1 ; N#Cc1ccc2c(c1)c1cc(C#N)ccc1n2-c1cccc(-n2c3ccccc3c3c4sc5ccccc5c4ccc32)c1]

### (1) Synthesis of Intermediate 5

Intermediate 5 (79%) was synthesized in the same manner as Synthesis of Intermediate 1 of Synthesis Example 1, except that 5H-benzo[4,5]thieno[3,2-c]carbazole was used instead of 12H-benzofuro[3,2-a]carbazole in synthesizing Intermediate 5.

### (2) Synthesis of Compound 44

Compound 44 (57%) was synthesized in the same manner as in Synthesis of Compound 27 of Synthesis Example 1, except that Intermediate 5 was used instead of Intermediate 1 in synthesizing Compound 44.

MALDI-TOF Mass (calculated value: 564.66 g/mol, measured value: 563.97 g/mol)

### Synthesis Example 6: Synthesis of Compound 45

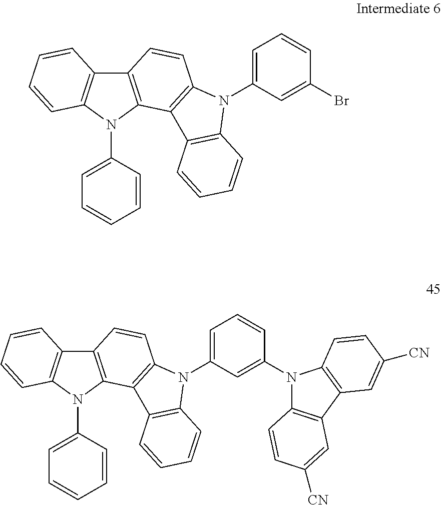 [SMILES: Brc1cccc(-n2c3ccccc3c3c2ccc2c4ccccc4n(-c4ccccc4)c23)c1 ; N#Cc1ccc2c(c1)c1cc(C#N)ccc1n2-c1cccc(-n2c3ccccc3c3c2ccc2c4ccccc4n(-c4ccccc4)c23)c1]

### (1) Synthesis of Intermediate 6

Intermediate 6 (17%) was synthesized in the same manner as in Synthesis of Intermediate 2 of Synthesis Example 2, except that, in synthesizing Intermediate 6, 12-phenyl-5,12-dihydroindolo[3,2-a]carbazole was used instead of 5-phenyl-5,12-dihydroindolo[3,2-a]carbazole, the solvent (DMF) was not used, 3-bromoiodobenzene was used in an excessive amount, and the temperature was 180° C. instead of 165° C.

### (2) Synthesis of Compound 45

Compound 45 (45%) was synthesized in the same manner as Synthesis of Compound 27 of Synthesis Example 1, except that Intermediate 6 was used instead of Intermediate 1 in synthesizing Compound 45.

MALDI-TOF Mass (calculated value: 623.70 g/mol, measured value: 623.41 g/mol)

### Synthesis Example 7: Synthesis of Compound 93

### (1) Synthesis of Intermediate 7

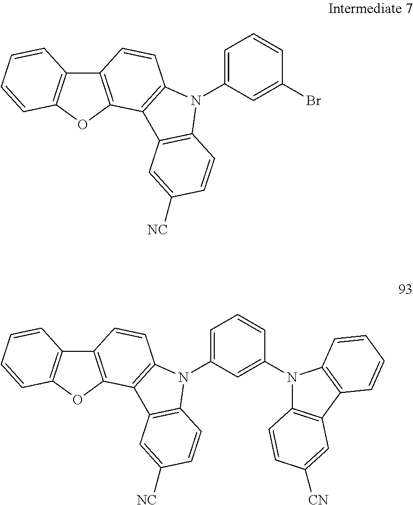 [SMILES: N#Cc1ccc2c(c1)c1c3oc4ccccc4c3ccc1n2-c1cccc(Br)c1 ; N#Cc1ccc2c(c1)c1ccccc1n2-c1cccc(-n2c3ccc(C#N)cc3c3c4oc5ccccc5c4ccc32)c1]

Intermediate 7 (78%) was synthesized in the same manner as in Synthesis of Intermediate 1 of Synthesis Example 1, except that 5H-benzofuro[3,2-c]carbazole-2-carbonitrile was used instead of 12H-benzofuro[3,2-a]carbazole in synthesizing Intermediate 7.

### (2) Synthesis of Compound 93

Compound 93 (62%) was synthesized in the same manner as in Synthesis of Compound 27 of Synthesis Example 1, except that Intermediate 7 was used instead of Intermediate 1 and 9H-carbazole-3-carbonitrile was used instead of 9H-carbazole-3,6-dicarbonitrile in synthesizing Compound 93.

MALDI-TOF Mass (calculated value: 548.59 g/mol, measured value: 548.12 g/mol)

### Synthesis Example 8: Synthesis of Compound 165

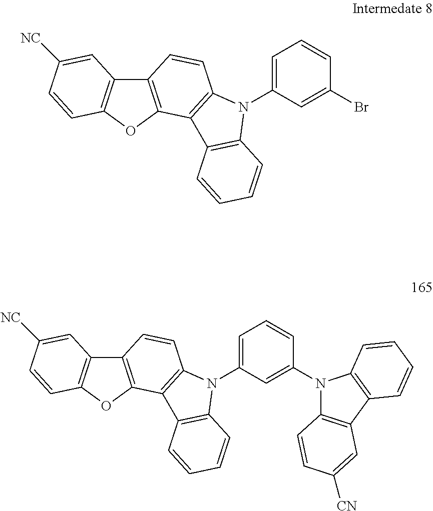 [SMILES: N#Cc1ccc2oc3c(ccc4c3c3ccccc3n4-c3cccc(Br)c3)c2c1 ; N#Cc1ccc2oc3c(ccc4c3c3ccccc3n4-c3cccc(-n4c5ccccc5c5cc(C#N)ccc54)c3)c2c1]

### (1) Synthesis of Intermediate 8

Intermediate 8 was synthesized in the same manner as in Synthesis of Intermediate 1 of Synthesis Example 1, except that 5H-benzofuro[3,2-c]carbazole-9-carbonitrile was used instead of 12H-benzofuro[3,2-a]carbazole in synthesizing Intermediate 8.

### (2) Synthesis of Compound 165

Compound 165 (54%) was synthesized in the same manner as in Synthesis of Compound 27 of Synthesis Example 1, except that Intermediate 8 was used instead of Intermediate 1 and 9H-carbazole-3-carbonitrile was used instead of 9H-carbazole-3,6-dicarbonitrile in synthesizing Compound 165.

MALDI-TOF Mass (calculated value: 548.59 g/mol, measured value: 548.12 g/mol)

### Synthesis Example 9: Synthesis of Compound 299

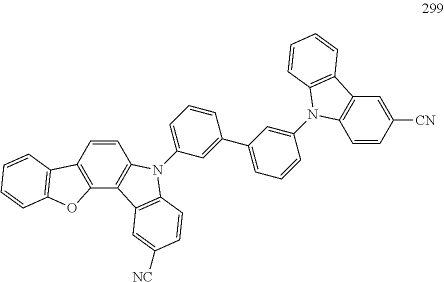 [SMILES: N#Cc1ccc2c(c1)c1ccccc1n2-c1cccc(-c2cccc(-n3c4ccc(C#N)cc4c4c5oc6ccccc6c5ccc43)c2)c1]

12 g (27.4 mmol) of Intermediate 7, 10.8 g (27.4 mmol) of 9-(3-(4,4,5,5-tetramethyl-1,3,2-dioxaborolan-2-yl)phenyl)-9H-carbazole-3-carbonitrile, 6.3 g (5.5 mmol) of Pd(PPh₃)₄, and 11.4 g (82 mmol) of K₂CO₃ were added into a 250-ml RBF, and 41 ml of water and 95 ml of tetrahydrofuran (THF) were added thereto. The mixture was stirred at a temperature of 90° C. for 21 hours. The reaction mixture was cooled, and methanol was poured thereto to obtain a precipitate. The precipitate was passed through a filtering paper. The obtained solid was dissolved in hot MC, and the solution was passed through a thin plug of silica. The filtrate was concentrated. The recrystallization with ethyl acetate was performed to obtain 6.9 g (40%) of Compound 299.

MALDI-TOF Mass (calculated value: 624.69 g/mol, measured value: 624.27 g/mol)

### Synthesis Example 10: Synthesis of Compound 665

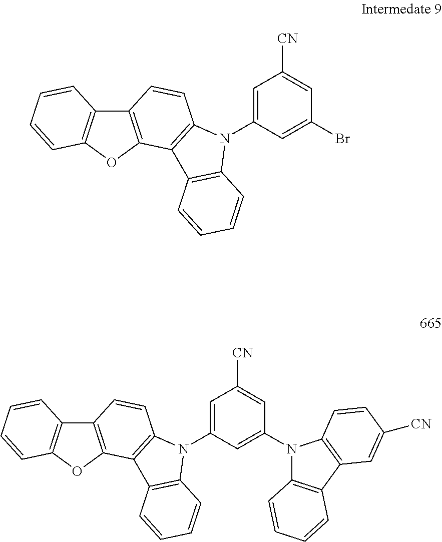 [SMILES: N#Cc1cc(Br)cc(-n2c3ccccc3c3c4oc5ccccc5c4ccc32)c1 ; N#Cc1cc(-n2c3ccccc3c3cc(C#N)ccc32)cc(-n2c3ccccc3c3c4oc5ccccc5c4ccc32)c1]

### (1) Synthesis of Intermediate 9

Intermediate 9 (71%) was synthesized in the same manner as in Intermediate 1 of Synthesis Example 1, except that 5H-benzofuro[3,2-c]carbazole was used instead of 12H-benzofuro[3,2-a]carbazole and 3-bromo-5-fluorobenzonitrile was used instead of 1-bromo-3-fluorobenzene in synthesizing Intermediate 9.

### (2) Synthesis of Compound 665

Compound 665 (62%) was synthesized in the same manner as in Synthesis of Compound 27 of Synthesis Example 1, except that Intermediate 9 was used instead of Intermediate 1 and 9H-carbazole-3-carbonitrile was used instead of 9H-carbazole-3,6-dicarbonitrile in synthesizing Compound 665.

MALDI-TOF Mass (calculated value: 548.59 g/mol, measured value: 548.18 g/mol)

### Synthesis Example 11: Synthesis of Compound 876

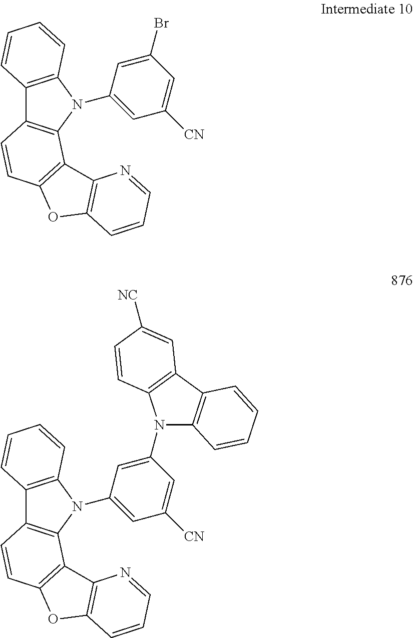 [SMILES: N#Cc1cc(Br)cc(-n2c3ccccc3c3ccc4oc5cccnc5c4c32)c1 ; N#Cc1cc(-n2c3ccccc3c3cc(C#N)ccc32)cc(-n2c3ccccc3c3ccc4oc5cccnc5c4c32)c1]

### (1) Synthesis of Intermediate 10

Intermediate 10 (59%) was synthesized in the same manner as in Synthesis of Intermediate 1 of Synthesis Example 1, except that 12H-pyrido[2′,3′:4,5]furo[3,2-a]carbazole was used instead of 12H-benzofuro[3,2-a]carbazole and 3-bromo-5-fluorobenzonitrile was used instead of 1-bromo-3-fluorobenzene in synthesizing Intermediate 10.

### (2) Synthesis of Compound 876

Compound 876 (63%) was synthesized in the same manner as in Synthesis of Compound 27 of Synthesis Example 1, except that Intermediate 10 was used instead of Intermediate 1 and 9H-carbazole-3-carbonitrile was used instead of 9H-carbazole-3,6-dicarbonitrile in synthesizing Compound 876.

MALDI-TOF Mass (calculated value: 549.58 g/mol, measured value: 549.18 g/mol)

### Evaluation Example 1: Evaluation of Thermal Characteristics

Thermal analysis (N₂ atmosphere, temperature range: from room temperature to 800-C(10° C./min)-TGA, from room temperature to 400-C-DSC, Pan Type: Pt Pan in disposable Al Pan (TGA), disposable Al pan (DSC)) was performed on Compounds 27, 29, 43, 44, A, B, and C by using thermogravimetric analysis (TGA) and differential scanning calorimetry (DSC). Results thereof are shown in Table 2.

TABLE 2

| Compound No. | Tg(° C.) |
|---|---|
| 27 | 159 |
| 29 | 185 |
| 43 | 166 |
| 44 | 177 |
| Compound A | 72 |
| Compound B | 130 |
| Compound C | 128 |

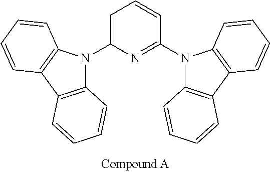 [SMILES: c1cc(-n2c3ccccc3c3ccccc32)nc(-n2c3ccccc3c3ccccc32)c1]

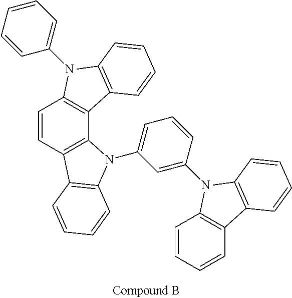 [SMILES: c1ccc(-n2c3ccccc3c3c2ccc2c4ccccc4n(-c4cccc(-n5c6ccccc6c6ccccc65)c4)c23)cc1]

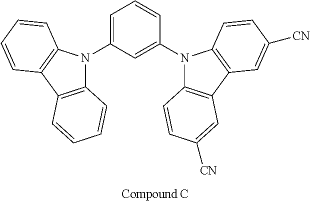 [SMILES: N#Cc1ccc2c(c1)c1cc(C#N)ccc1n2-c1cccc(-n2c3ccccc3c3ccccc32)c1]

Referring to Table 2, it was confirmed that Compounds 27, 29, 43, and 44 had excellent thermal stability as compared with Compounds A, B, and C.

### Example 1

A glass substrate, on which an ITO electrode (a first electrode or an anode) was formed to have a thickness of 1,500 Å, was ultrasonically washed with distilled water. When the washing with distilled water was completed, sonification washing was performed using a solvent, such as iso-propyl alcohol, acetone, or methanol. The resultant washed glass substrate was dried and transferred to a plasma washer in which the glass substrate was washed with oxygen plasma for 5 minutes, and then, the glass substrate was transferred to a vacuum-depositing device.

Compound HT3 and Compound HP-1 were co-deposited on the ITO electrode of the glass substrate to form a hole injection layer having a thickness of 100 Å, and Compound HT3 was deposited on the hole injection layer to form a hole transport layer having a thickness of 1,300 Å. Then, mCP was deposited on the hole transport layer to form an electron blocking layer having a thickness of 150 Å, thereby forming a hole transport region.

Compound 27 (host) and FIr6 (dopant, 10 percent by weight, wt %) were co-deposited on the hole transport region to form an emission layer having a thickness of 300 Å.

Compound 1 was vacuum-deposited on the emission layer to form a hole blocking layer having a thickness of 100 Å, and Compound ET3 and LiQ were vacuum-deposited on the hole blocking layer to form an electron transport layer having a thickness of 250 Å. Then, LiQ was deposited on the electron transport layer to form an electron injection layer having a thickness of 5 Å, and an Al electrode (cathode) having a thickness of 1,000 Å was formed on the electron injection layer, thereby completing the manufacture of an organic light-emitting device.

### Examples 2 to 6 and Comparative Examples 1 and 2

Organic light-emitting devices of Examples 2 to 6 and Comparative Examples 1 and 2 were manufactured in the same manner as in Example 1, except that Compounds shown in Table 3 were used instead of Compound 27 as a material for a host of an emission layer.

### Evaluation Example 4: Evaluation on Characteristics of Organic Light-Emitting Devices

The efficiency and lifespan (T₈₀) of the organic light-emitting devices manufactured in Examples 1 to 6 and Comparative Examples 1 and 2 were evaluated using a Keithley SMU 236 and a PR650 luminance meter. In Table 3, the efficiency of Examples 1 to 6 and Comparative Examples 1 and 2 is a relative value converted when the efficiency of the organic light-emitting device of Compound A is assumed as “100”.

In Table 3, the lifespan (T₈₀) was obtained by measuring a period of time (hours, hr) that had lapsed until the luminance was reduced to 80% of the initial luminance (@500nit) after driving of the organic light-emitting device. In Table 3, the lifespan (T₈₀) of Examples 1 to 6 and Comparative Examples 1 and 2 is a relative value converted when the lifespan (T₈₀) of the organic light-emitting device of Compound A is assumed as “100”.

TABLE 3

| | | Efficiency | |
|---|---|---|---|
| | Material for host | (cd/A) | T₈₀ (hr) |
| | of emission layer | (relative value) | (relative value) |
| Example 1 | Compound 27 | 108 | 389 |
| Example 2 | Compound 29 | 170 | 204 |
| Example 3 | Compound 43 | 134 | 351 |
| Example 4 | Compound 44 | 133 | 189 |
| Example 5 | Compound 93 | 120 | 250 |
| Example 6 | Compound 299 | 155 | 275 |
| Comparative | Compound A | 100 | 100 |
| Example 1 | | | |
| Comparative | Compound B | 124 | 243 |
| Example 2 | | | |

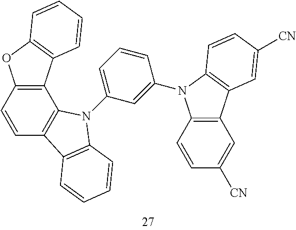 [SMILES: N#Cc1ccc2c(c1)c1cc(C#N)ccc1n2-c1cccc(-n2c3ccccc3c3ccc4oc5ccccc5c4c32)c1]

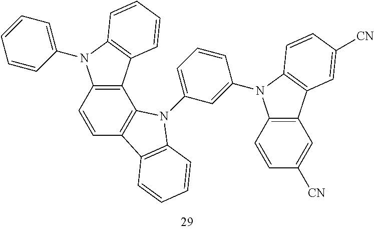 [SMILES: N#Cc1ccc2c(c1)c1cc(C#N)ccc1n2-c1cccc(-n2c3ccccc3c3ccc4c(c5ccccc5n4-c4ccccc4)c32)c1]

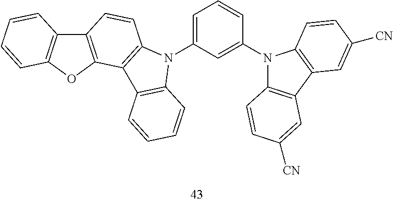 [SMILES: N#Cc1ccc2c(c1)c1cc(C#N)ccc1n2-c1cccc(-n2c3ccccc3c3c4oc5ccccc5c4ccc32)c1]

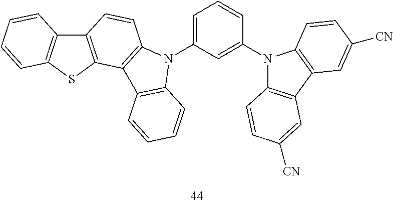 [SMILES: N#Cc1ccc2c(c1)c1cc(C#N)ccc1n2-c1cccc(-n2c3ccccc3c3c4sc5ccccc5c4ccc32)c1]

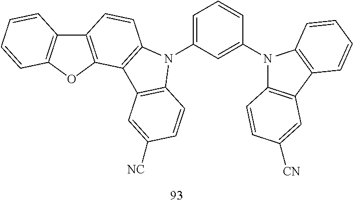 [SMILES: N#Cc1ccc2c(c1)c1ccccc1n2-c1cccc(-n2c3ccc(C#N)cc3c3c4oc5ccccc5c4ccc32)c1]

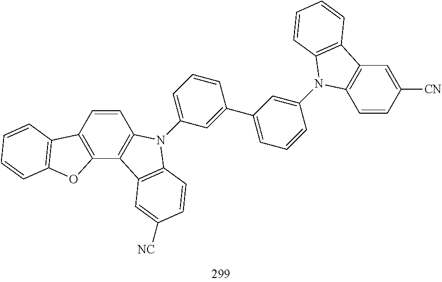 [SMILES: N#Cc1ccc2c(c1)c1ccccc1n2-c1cccc(-c2cccc(-n3c4ccc(C#N)cc4c4c5oc6ccccc6c5ccc43)c2)c1]

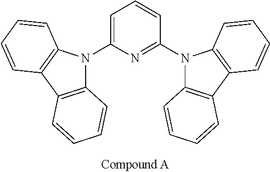 [SMILES: c1cc(-n2c3ccccc3c3ccccc32)nc(-n2c3ccccc3c3ccccc32)c1]

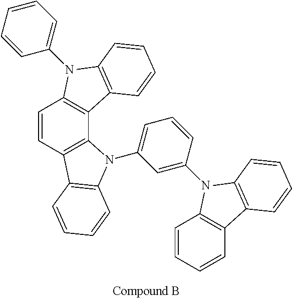 [SMILES: c1ccc(-n2c3ccccc3c3c2ccc2c4ccccc4n(-c4cccc(-n5c6ccccc6c6ccccc65)c4)c23)cc1]

Referring to Table 3, it was confirmed that the organic light-emitting devices of Examples 1 to 6 had high efficiency and a long lifespan as compared to those of the organic light-emitting devices of Comparative Examples 1 and 2.

Since the condensed cyclic compound has excellent electrical characteristics and thermal stability, the organic light-emitting device including the condensed cyclic compound may have high efficiency and long lifespan characteristics.

It should be understood that embodiments described herein should be considered in a descriptive sense only and not for purposes of limitation. Descriptions of features or aspects within each embodiment should typically be considered as available for other similar features or aspects in other embodiments.

While one or more embodiments have been described with reference to the figures, it will be understood by those of ordinary skill in the art that various changes in form and details may be made therein without departing from the spirit and scope of the present disclosure as defined by the following claims.

What is claimed is:

1. A condensed cyclic compound represented by Formula 1: 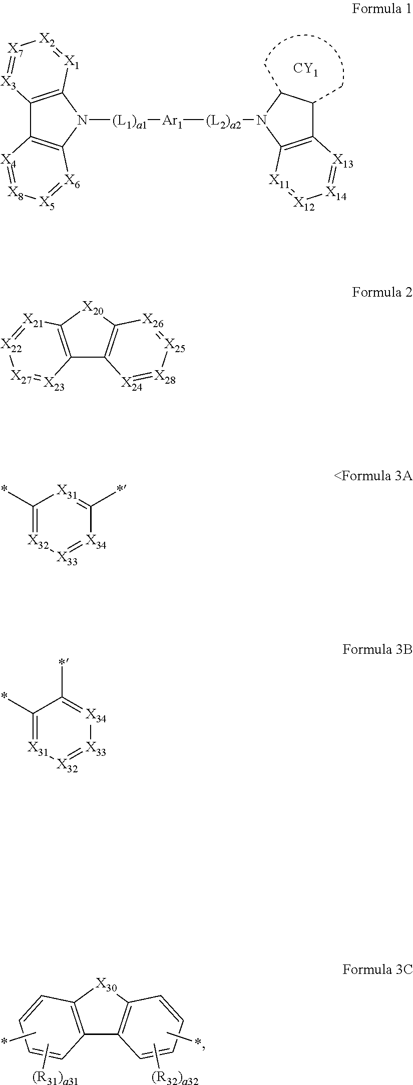
wherein, in Formulae 1, 2, and 3A to 3C,
X₁ is N or C(R₁), X₂ is N or C(R₂), X₃ is N or C(R₃), X₄ is N or C(R₄), X₅ is N or C(R₅), X₆ is N or C(R₆), X₇ is N or C(R₇), and X₈ is N or C(R₈),
X₁₁ is N or C(R₁₁), X₁₂ is N or C(R₁₂), X₁₃ is N or C(R₁₃), and X₁₄ is N or C(R₁₄),
CY₁ is represented by Formula 2,
X₂₀ is selected from O, S, N(R₂₀), and C(R₂₀)(R₂₉),
X₂₁ is N or C(R₂₁), X₂₂ is N or C(R₂₂), X₂₃ is N or C(R₂₃), X₂₄ is N or C(R₂₄), X₂₅ is N or C(R₂₅), X₂₆ is N or C(R₂₆), X₂₇ is N or C(R₂₇), and X₂₈ is N or C(R₂₈),
CY₁ is fused with a neighboring 5-membered ring comprising N as a ring-forming atom via X₂₁ and X₂₂, X₂₂ and X₂₇, X₂₇ and X₂₃, X₂₄ and X₂₈, X₂₈ and X₂₅, or X₂₅ and X₂₆,
R₁ to R₈, R₁₁ to R₁₄, and R₂₀ to R₂₉ are each independently selected from hydrogen, deuterium, —F, —Cl, —Br, —I, a hydroxyl group, a cyano (CN) group, a nitro group, an amino group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent non-aromatic condensed polycyclic group, a substituted or unsubstituted monovalent non-aromatic condensed heteropolycyclic group, and —Si(Q₁)(Q₂)(Q₃),
at least one selected from X₇, X₈, X₁₄, and X₂₁ to X₂₈ is C(CN),
Ar₁ is represented by one selected from Formulae 3A to 3C,
X₃₀ is selected from O, S, N(R₃₀), C(R₃₀)(R₃₅), Si(R₃₀)(R₃₅), Se, and P(═O)(R₃₀),
X₃₁ is N or C(R₃₁), X₃₂ is N or C(R₃₂), X₃₃ is N or C(R₃₃), and X₃₄ is N or C(R₃₄),
R₃₀ to R₃₅ are each independently selected from hydrogen, deuterium, a cyano group, a C₁-C₄ alkyl group, a phenyl group, a pyridinyl group, a pyrimidinyl group, a pyrazinyl group, a pyridazinyl group, a triazinyl group, and —Si(Q₁₁)(Q₁₂)(Q₁₃),
a31 and a32 are each independently an integer selected from 0 to 3, wherein when a31 is two or more, two or more groups R₃₁ are identical to or different from each other, and when a32 is two or more, two or more groups R₃₂ are identical to or different from each other,
L₁ and L₂ are each independently selected from:
a phenylene group, a pyridinylene group, a pyrimidinylene group, a pyrazinylene group, a pyridazinylene group, a triazinylene group, a dibenzofuranylene group, a dibenzothiophenylene group, and a carbazolylene group; and
a phenylene group, a pyridinylene group, a pyrimidinylene group, a pyrazinylene group, a pyridazinylene group, a triazinylene group, a dibenzofuranylene group, a dibenzothiophenylene group, and a carbazolylene group, each substituted with at least one selected from deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₁₀ alkyl group, a C₁-C₁₀ alkoxy group, a phenyl group, a naphthyl group, a pyridinyl group, a pyrimidinyl group, a pyrazinyl group, a pyridazinyl group, a triazinyl group, and —Si(Q₂₁)(Q₂₂)(Q₂₃),
a1 and a2 are each independently an integer selected from 0 to 5, wherein when a1 is two or more, two or more groups L₁ are identical to or different from each other, and when a2 is two or more, two or more groups L₂ are identical to or different from each other,
when Ar₁ is represented by Formula 3A or 3B, the sum of a1 and a2 is 1, and L₁ or L₂ is a phenylene group, a group represented by *-(L₁)ₐ₁-Ar₁-(L₂)ₐ₂-*′ in Formula 1 does not comprise a cyano (CN) group as a substituent,
* and *′ each indicate a binding side to a neighboring atom, and
at least one substituent selected from a substituent(s) of the substituted C₁-C₆₀ alkyl group, the substituted C₂-C₆₀ alkenyl group, the substituted C₂-C₆₀ alkynyl group, the substituted C₁-C₆₀ alkoxy group, the substituted C₃-C₁₀ cycloalkyl group, the substituted C₁-C₁₀ heterocycloalkyl group, the substituted C₃-C₁₀ cycloalkenyl group, the substituted C₁-C₁₀ heterocycloalkenyl group, the substituted C₆-C₆₀ aryl group, the substituted C₆-C₆₀ aryloxy group, the substituted C₆-C₆₀ arylthio group, the substituted C₁-C₆₀ heteroaryl group, the substituted monovalent non-aromatic condensed polycyclic group, and the substituted monovalent non-aromatic condensed heteropolycyclic group is selected from deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, and —Si(Q₃₁)(Q₃₂)(Q₃₃),
wherein Q₁ to Q₃, Q₁₁ to Q₁₃, Q₂₁ to Q₂₃, and Q₃₁ to Q₃₃ are each independently selected from hydrogen, a C₁-C₆₀ alkyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₁-C₆₀ heteroaryl group, a monovalent non-aromatic condensed polycyclic group, and a monovalent non-aromatic condensed heteropolycyclic group.

2. The condensed cyclic compound of claim 1, wherein,
in Formulae 1 and 2,
R₁ to R₈, R₁₁ to R₁₄, and R₂₀ to R₂₉ are each independently selected from:
hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, and a C₁-C₂₀ alkoxy group;
a C₁-C₂₀ alkyl group and a C₁-C₂₀ alkoxy group, each substituted with at least one selected from deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a phenyl group, a naphthyl group, a pyridinyl group, a pyrimidinyl group, a pyrazinyl group, a pyridazinyl group, and a triazinyl group;
a cyclopentyl group, a cyclohexyl group, a cyclopentenyl group, a cyclohexenyl group, a cycloheptenyl group, a phenyl group, a pentalenyl group, an indenyl group, a naphthyl group, an azulenyl group, a heptalenyl group, an indacenyl group, an acenaphthyl group, a fluorenyl group, a spiro-fluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a perylenyl group, a pentaphenyl group, a hexacenyl group, a pyrrolyl group, an imidazolyl group, a pyrazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a phenanthridinyl group, an acridinyl group, a phenanthrolinyl group, a phenazinyl group, a benzoxazolyl group, a benzimidazolyl group, a furanyl group, a benzofuranyl group, a thiophenyl group, a benzothiophenyl group, a thiazolyl group, an isothiazolyl group, a benzothiazolyl group, an isoxazolyl group, an oxazolyl group, a triazolyl group, a tetrazolyl group, an oxadiazolyl group, a triazinyl group, a dibenzofuranyl group, a dibenzothiophenyl group, an imidazopyridimidinyl group, and an imidazopyridinyl group;
a cyclopentyl group, a cyclohexyl group, a cyclopentenyl group, a cyclohexenyl group, a cycloheptenyl group, a phenyl group, a pentalenyl group, an indenyl group, a naphthyl group, an azulenyl group, a heptalenyl group, an indacenyl group, an acenaphthyl group, a fluorenyl group, a spiro-fluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a perylenyl group, a pentaphenyl group, a hexacenyl group, a pyrrolyl group, an imidazolyl group, a pyrazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a phenanthridinyl group, an acridinyl group, a phenanthrolinyl group, a phenazinyl group, a benzoxazolyl group, a benzimidazolyl group, a furanyl group, a benzofuranyl group, a thiophenyl group, a benzothiophenyl group, a thiazolyl group, an isothiazolyl group, a benzothiazolyl group, an isoxazolyl group, an oxazolyl group, a triazolyl group, a tetrazolyl group, an oxadiazolyl group, a triazinyl group, a dibenzofuranyl group, a dibenzothiophenyl group, an imidazopyridimidinyl group, and an imidazopyridinyl group, each substituted with at least one selected from deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₂-C₂₀ alkenyl group, a C₂-C₂₀ alkynyl group, a C₁-C₂₀ alkoxy group, a phenyl group, a naphthyl group, an anthracenyl group, a pyrenyl group, a phenanthrenyl group, a fluorenyl group, a pyridinyl group, a pyrimidinyl group, a pyrazinyl group, a pyridazinyl group, a triazinyl group, a quinolinyl group, an isoquinolinyl group, a phthalazinyl group, a quinoxalinyl group, a cinnolinyl group, a quinazolinyl group, and —Si(Q₃₁)(Q₃₂)(Q₃₃); and
—Si(Q₁)(Q₂)(Q₃),
wherein Q₁ to Q₃ and Q₃₁ to Q₃₃ are each independently selected from hydrogen, a C₁-C₂₀ alkyl group, a C₁-C₂₀ alkoxy group, a phenyl group, a naphthyl group, an anthracenyl group, a pyrenyl group, a phenanthrenyl group, a fluorenyl group, a pyridinyl group, a pyrimidinyl group, a pyrazinyl group, a pyridazinyl group, a triazinyl group, a quinolinyl group, an isoquinolinyl group, a phthalazinyl group, a quinoxalinyl group, a cinnolinyl group, and a quinazolinyl group.

3. The condensed cyclic compound of claim 1, wherein,
in Formulae 1 and 2,
R₁ to R₆, R₁₁ to R₁₃, R₂₀ to R₂₆, and R₂₉ are each independently selected from:
hydrogen, deuterium, —F, —Cl, —Br, —I, a hydroxyl group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₁₀ alkyl group, and a C₁-C₁₀ alkoxy group;
a phenyl group, a naphthyl group, a pyridinyl group, a pyrimidinyl group, a pyrazinyl group, a pyridazinyl group, and a triazinyl group;
a phenyl group, a naphthyl group, a pyridinyl group, a pyrimidinyl group, a pyrazinyl group, a pyridazinyl group, and a triazinyl group, each substituted with at least one selected from deuterium, —F, —Cl, —Br, —I, a hydroxyl group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₁₀ alkyl group, a C₁-C₁₀ alkoxy group, a phenyl group, a naphthyl group, a pyridinyl group, a pyrimidinyl group, a pyrazinyl group, a pyridazinyl group, a triazinyl group, and —Si(Q₃₁)(Q₃₂)(Q₃₃); and
—Si(Q₁)(Q₂)(Q₃), and
R₇, R₈, R₁₄, R₂₇, and R₂₈ are each independently selected from:
hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₁₀ alkyl group, and a C₁-C₁₀ alkoxy group;
a phenyl group, a naphthyl group, a pyridinyl group, a pyrimidinyl group, a pyrazinyl group, a pyridazinyl group, and a triazinyl group;
a phenyl group, a naphthyl group, a pyridinyl group, a pyrimidinyl group, a pyrazinyl group, a pyridazinyl group, and a triazinyl group, each substituted with at least one selected from deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₁₀ alkyl group, a C₁-C₁₀ alkoxy group, a phenyl group, a naphthyl group, a pyridinyl group, a pyrimidinyl group, a pyrazinyl group, a pyridazinyl group, a triazinyl group, and —Si(Q₃₁)(Q₃₂)(Q₃₃); and
—Si(Q₁)(Q₂)(Q₃),
wherein Q₁ to Q₃ and Q₃₁ to Q₃₃ are each independently selected from hydrogen, a C₁-C₁₀ alkyl group, a C₁-C₁₀ alkoxy group, a phenyl group, a naphthyl group, a pyridinyl group, a pyrimidinyl group, a pyrazinyl group, a pyridazinyl group, and a triazinyl group.

4. The condensed cyclic compound of claim 1, wherein
at least one selected from X₇, X₈, X₁₄, X₂₁, and X₂₆ to X₂₈ in Formulae 1 and 2 is C(CN).

5. The condensed cyclic compound of claim 1, wherein
X₁ to X₆, X₁₁ to X₁₃, and X₂₂ to X₂₅ in Formulae 1 and 2 are not C(CN).

6. The condensed cyclic compound of claim 1, wherein
Ar₁ in Formula 1 is represented by one selected from Formulae 3A-1 to 3A-10, 3B-1 to 3B-8, and 3C-1 to 3C-9: 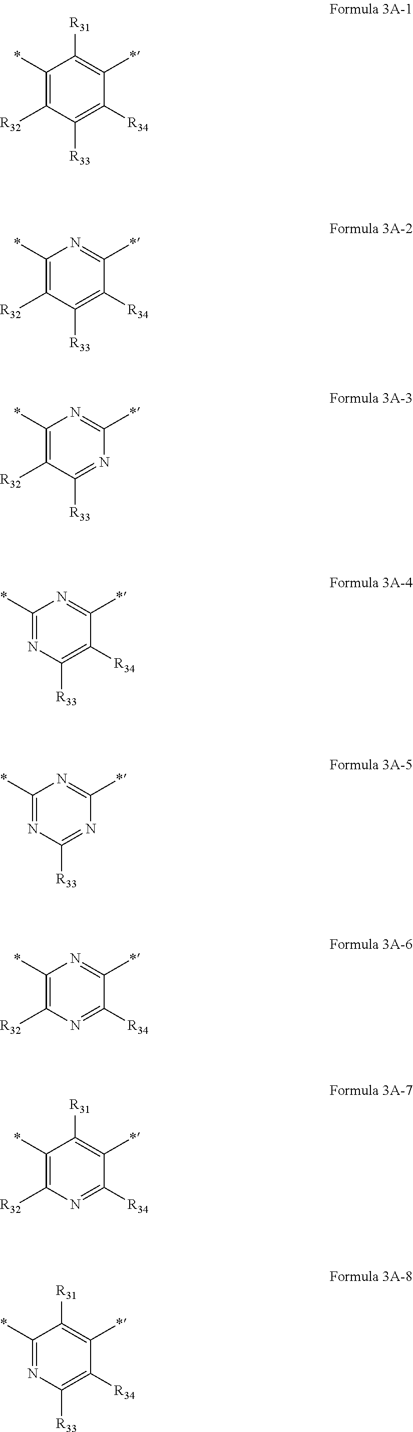 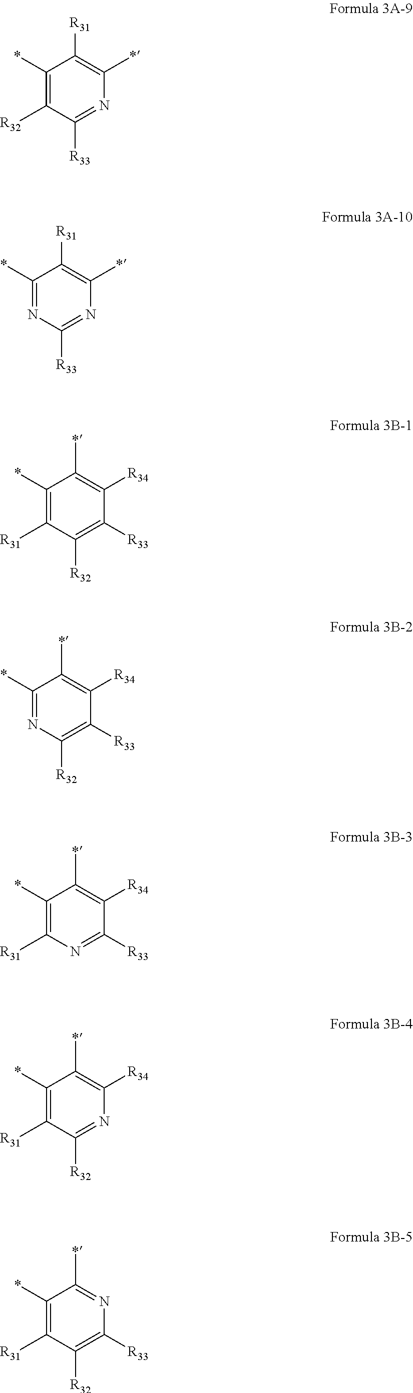 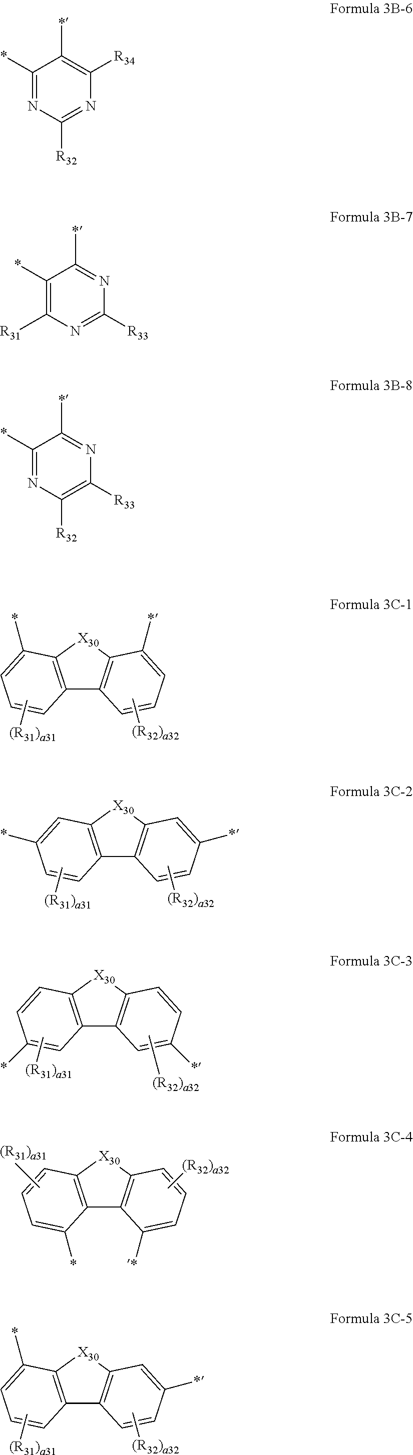 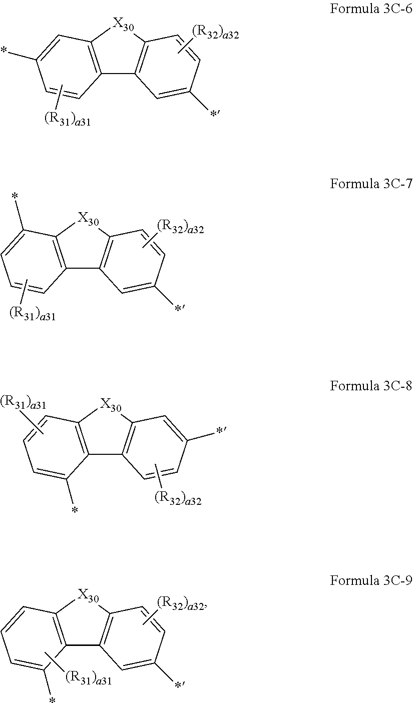
wherein, in Formulae 3A-1 to 3A-10, 3B-1 to 3B-8, and 3C-1 to 3C-9,
X₃₀ is selected from O, S, N(R₃₀), C(R₃₀)(R₃₅), Si(R₃₀)(R₃₅), Se, and P(═O)(R₃₀),
R₃₁ to R₃₄ are each independently selected from hydrogen, deuterium, a cyano group, a methyl group, an ethyl group, an n-propyl group, an iso-propyl group, an n-butyl group, an iso-butyl group, a sec-butyl group, a tert-butyl group, a phenyl group, a pyridinyl group, a pyrimidinyl group, a pyrazinyl group, a pyridazinyl group, a triazinyl group, and —Si(Q₁₁)(Q₁₂)(Q₁₃),
R₃₀ and R₃₅ are each independently selected from hydrogen, deuterium, a cyano group, a methyl group, an ethyl group, an n-propyl group, an iso-propyl group, an n-butyl group, an iso-butyl group, a sec-butyl group, a tert-butyl group, a phenyl group, a pyridinyl group, a pyrimidinyl group, a pyrazinyl group, a pyridazinyl group, and a triazinyl group,
wherein Q₁₁ to Q₁₃ are each independently selected from hydrogen, a C₁-C₁₀ alkyl group, a C₁-C₁₀ alkoxy group, and a phenyl group,
a31 and a32 are each independently 0 or 1, and
* and *′ each indicate a binding site to a neighboring atom.

7. The condensed cyclic compound of claim 1, wherein
L₁ and L₂ in Formula 1 are each independently selected from:
a phenylene group, a pyridinylene group, a pyrimidinylene group, and a triazinylene group; and
a phenylene group, a pyridinylene group, a pyrimidinylene group, and a triazinylene group, each substituted with at least one selected from deuterium, a cyano group, a methyl group, an ethyl group, an n-propyl group, an iso-propyl group, an n-butyl group, an iso-butyl group, a sec-butyl group, a tert-butyl group, a phenyl group, a pyridinyl group, a pyrimidinyl group, a pyrazinyl group, a pyridazinyl group, a triazinyl group, and —Si(Q₂₁)(Q₂₂)(Q₂₃).

8. The condensed cyclic compound of claim 1, wherein
L₁ and L₂ in Formula 1 are each independently selected from:
a phenylene group; and
a phenylene group substituted with at least one selected from deuterium, a cyano group, a methyl group, an ethyl group, an n-propyl group, an iso-propyl group, an n-butyl group, an iso-butyl group, a sec-butyl group, a tert-butyl group, a phenyl group, a pyridinyl group, a pyrimidinyl group, a pyrazinyl group, a pyridazinyl group, a triazinyl group, and —Si(Q₂₁)(Q₂₂)(Q₂₃),
wherein Q₂₁ to Q₂₃ are each independently selected from hydrogen, a C₁-C₁₀ alkyl group, a C₁-C₁₀ alkoxy group, and a phenyl group, and
a1 and a2 are each independently 0 or 1.

9. The condensed cyclic compound of claim 6, wherein,
in Formula 1, a1 and a2 are 0, and Ar₁ is selected from groups represented by Formulae 3A-1, 3A-2, 3B-1, and 3C-3.

10. The condensed cyclic compound of claim 1, wherein,
in Formula 1, the sum of a1 and a2 is one or more, and *-(L₁)ₐ₁-Ar₁-(L₂)ₐ₂-*′ is represented by one selected from Formulae 3-1 to 3-57: 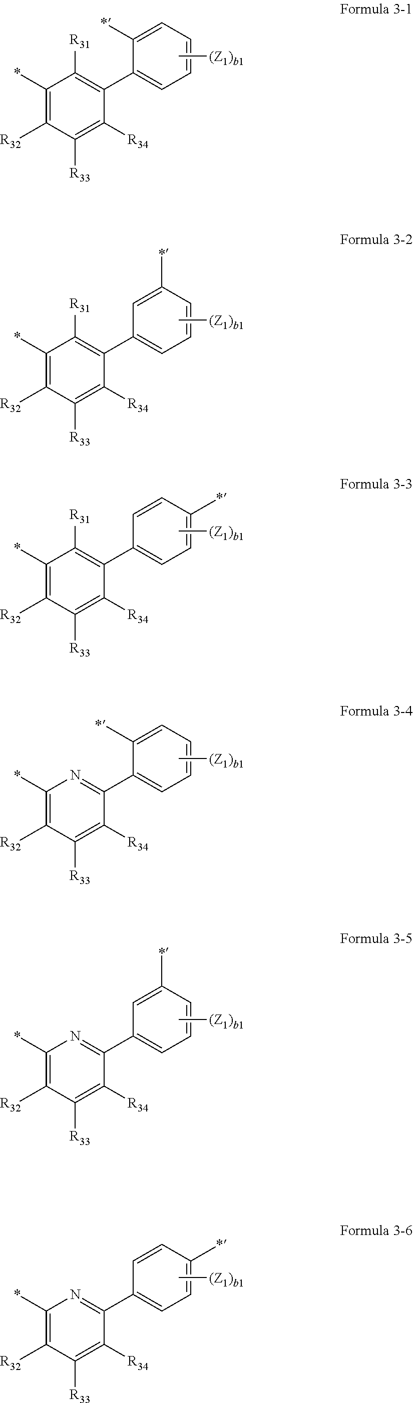 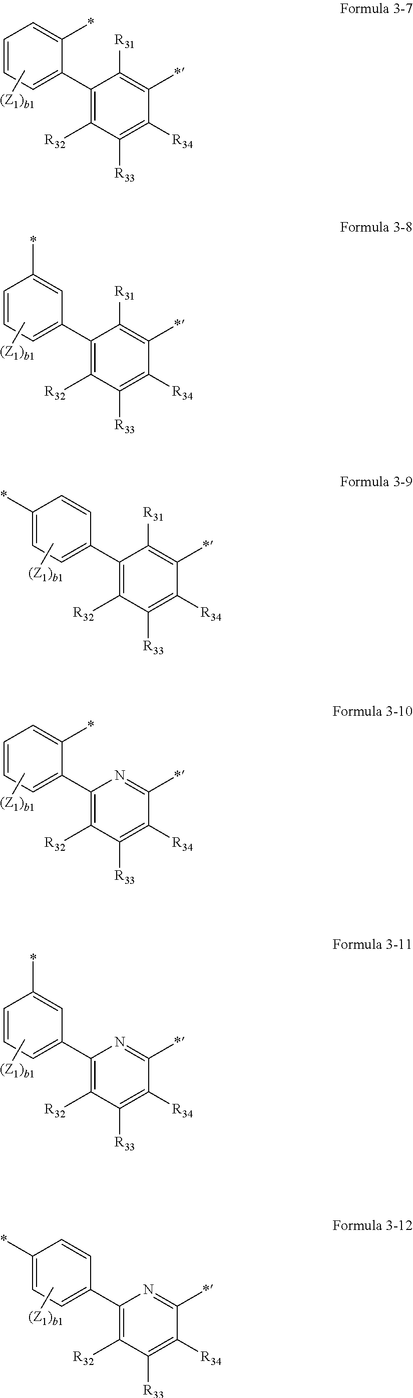 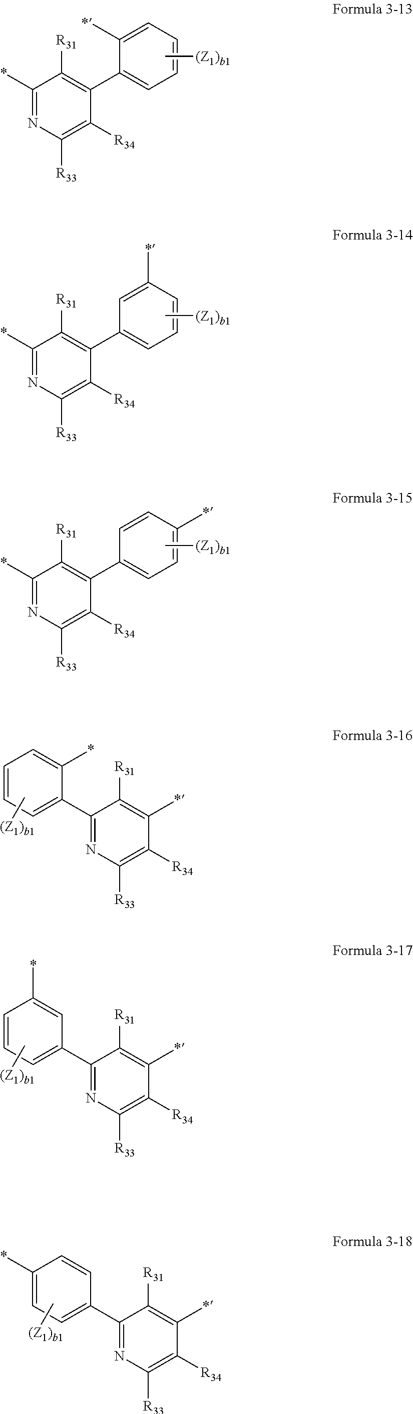 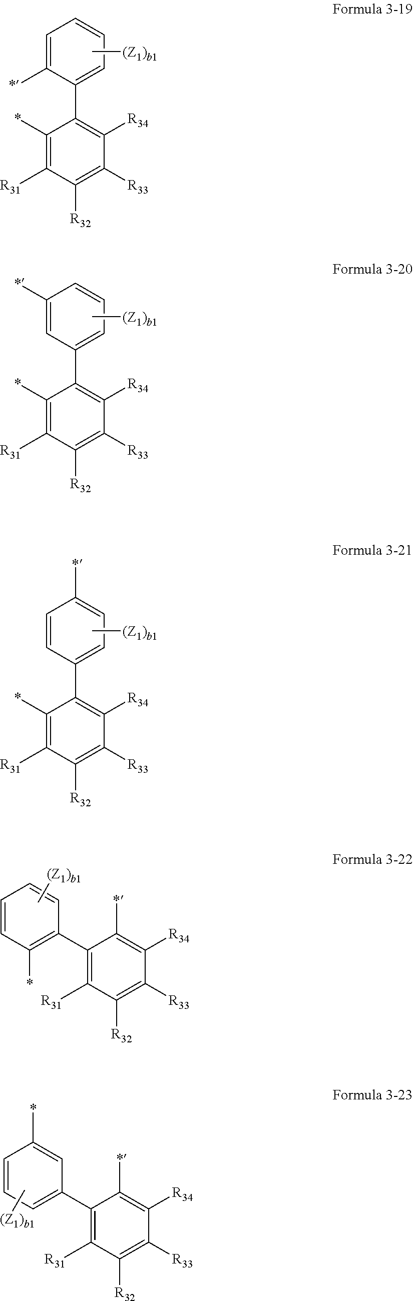 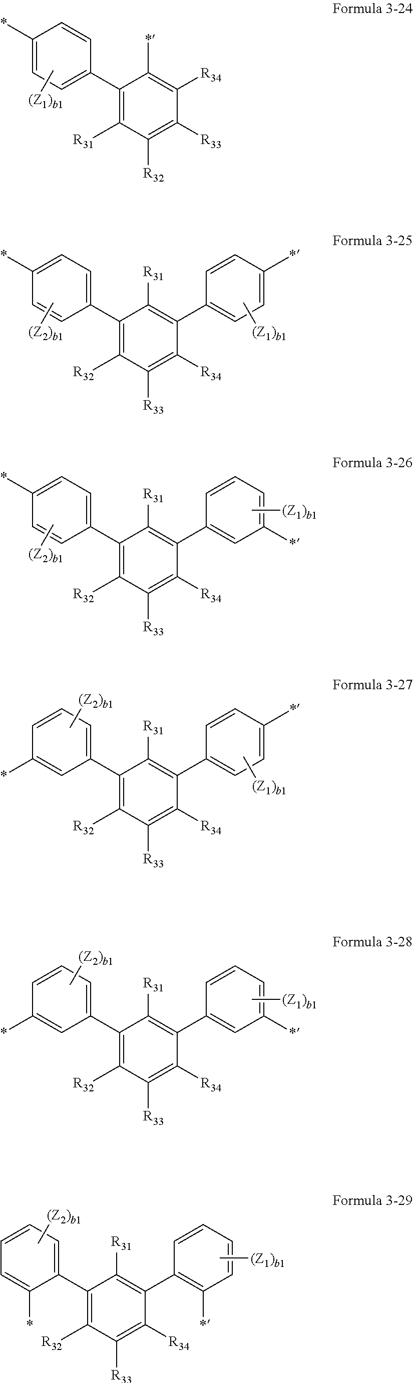 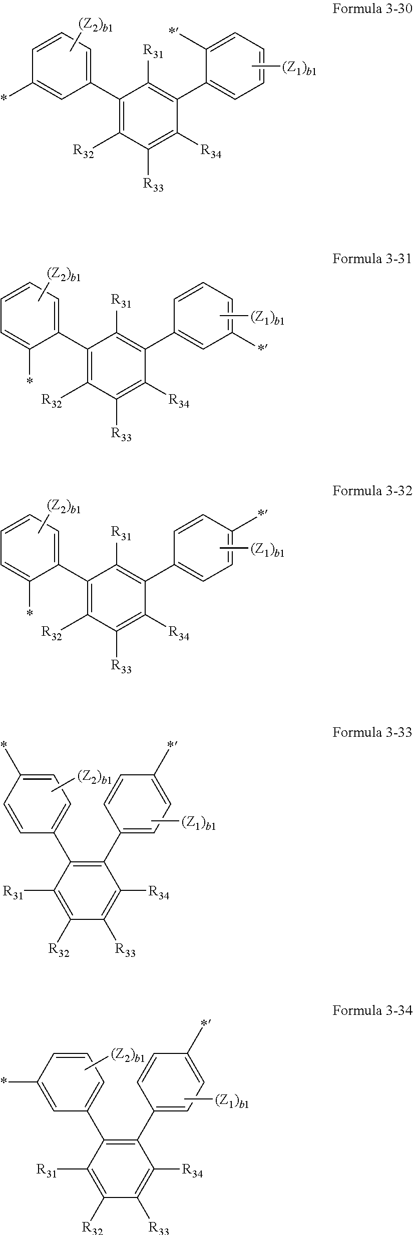 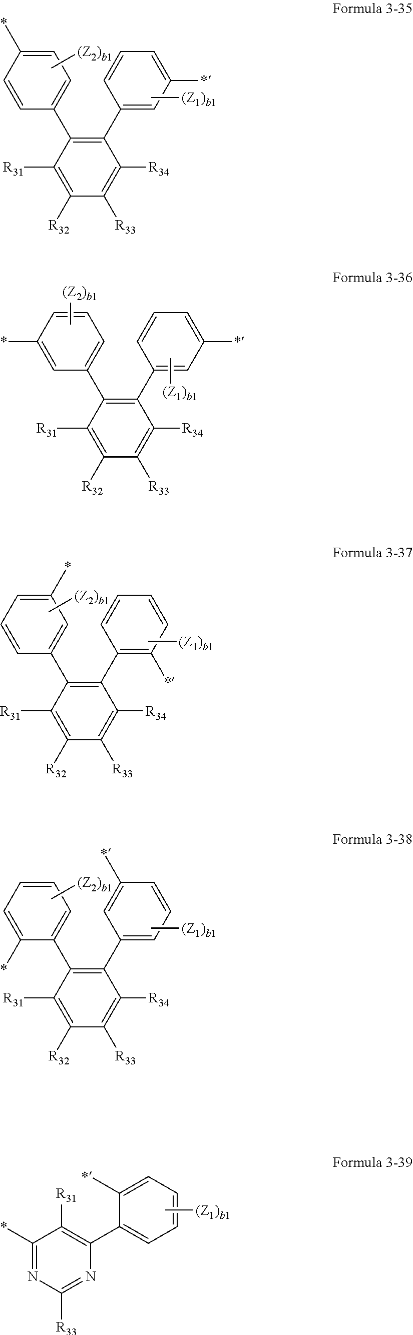 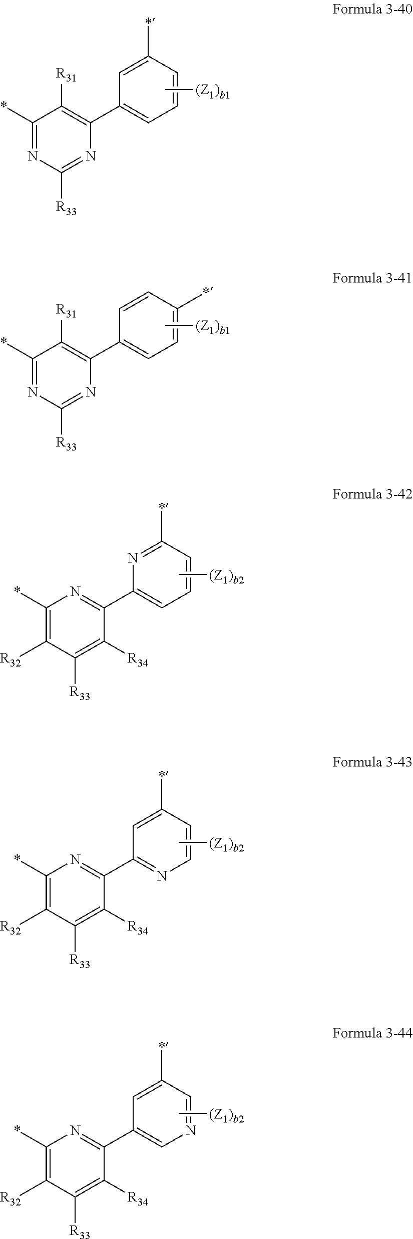 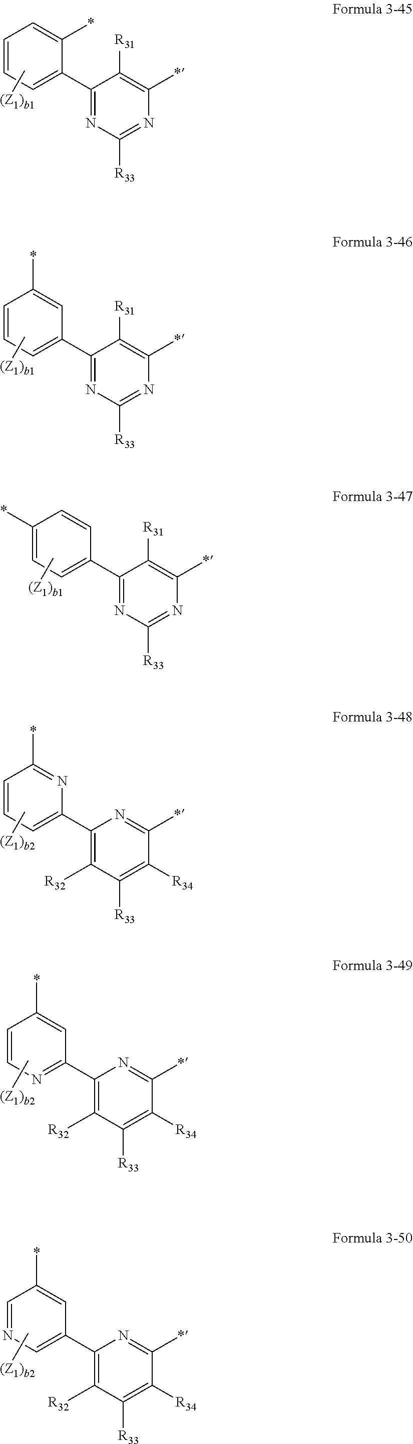 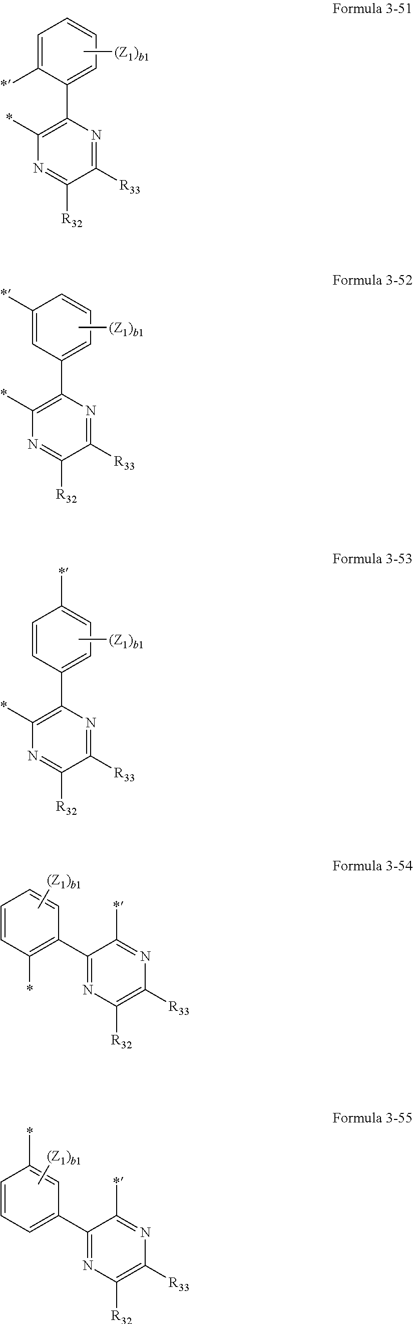 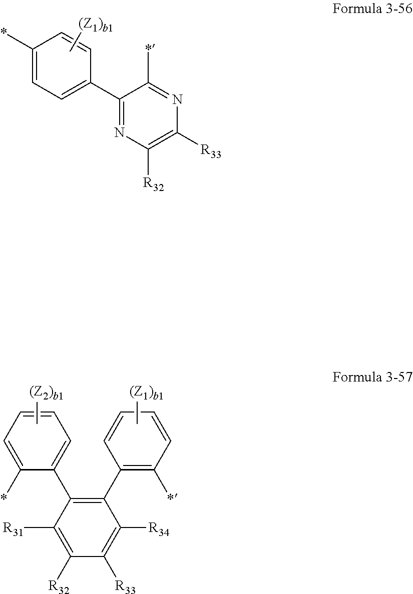
wherein, in Formulae 3-1 to 3-57,
R₃₁ to R₃₄, Z₁, and Z₂ are each independently selected from hydrogen, deuterium, a cyano group, a methyl group, an ethyl group, an n-propyl group, an iso-propyl group, an n-butyl group, an iso-butyl group, a sec-butyl group, a tert-butyl group, a phenyl group, a pyridinyl group, a pyrimidinyl group, a pyrazinyl group, a pyridazinyl group, a triazinyl group, and —Si(Q₁₁)(Q₁₂)(Q₁₃),
wherein Q₁₁ to Q₁₃ are each independently selected from hydrogen, a C₁-C₁₀ alkyl group, a C₁-C₁₀ alkoxy group, and a phenyl group,
b1 is an integer selected from 0 to 4, and b2 is an integer selected from 0 to 3, and
* and *′ each indicate a binding site to a neighboring nitrogen atom.

11. The condensed cyclic compound of claim 1, wherein the condensed cyclic compound is represented by one selected from Formulae 1(1) to 1(7): 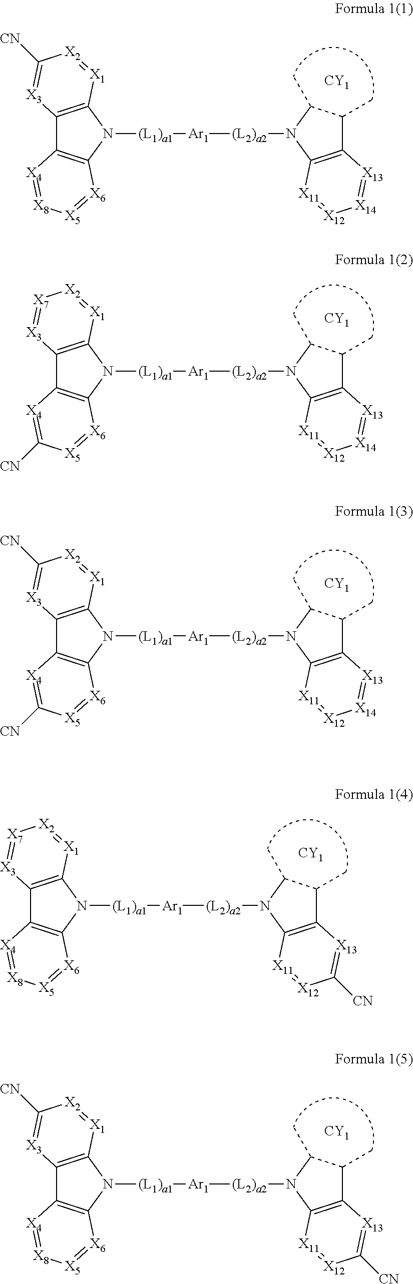 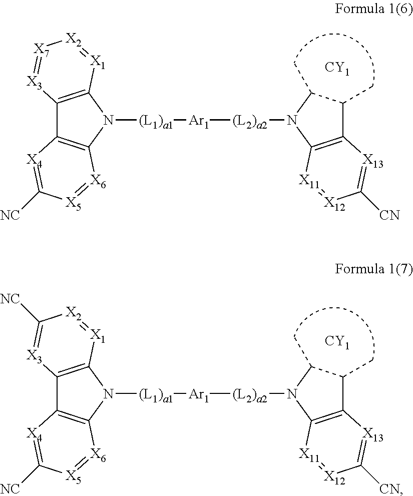
wherein X₁ to X₈, X₁₁ to X₁₄, CY₁, Ar₁, L₁, L₂, a1, and a2 in Formulae 1(1) to 1(7) are the same as described in claim 1.

12. The condensed cyclic compound of claim 1, wherein
the condensed cyclic compound is represented by one selected from Formulae 1A to 1F: 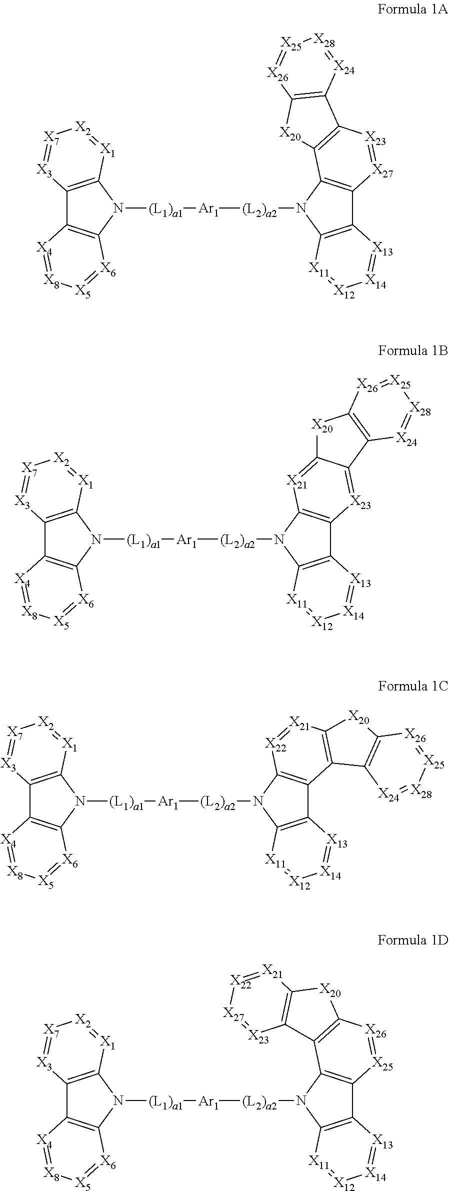 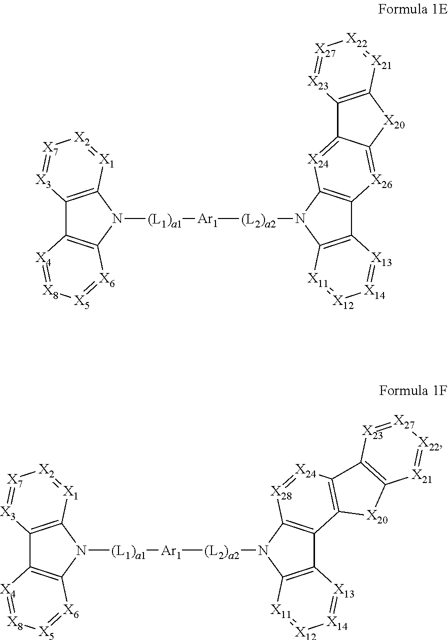
wherein X₁ to X₈, X₁₁ to X₁₄, X₂₀ to X₂₈, Ar₁, L₁, L₂, a1, and a2 in Formulae 1A to 1F are the same as described in claim 1.

13. The condensed cyclic compound of claim 1, wherein
the condensed cyclic compound is represented by one selected from Formulae 1A(1), 1A(2), 1B(1), 1B(2), 1C(1), 1C(2), 1D(1), 1D(2), 1E(1), 1E(2), 1F(1), and 1F(2): 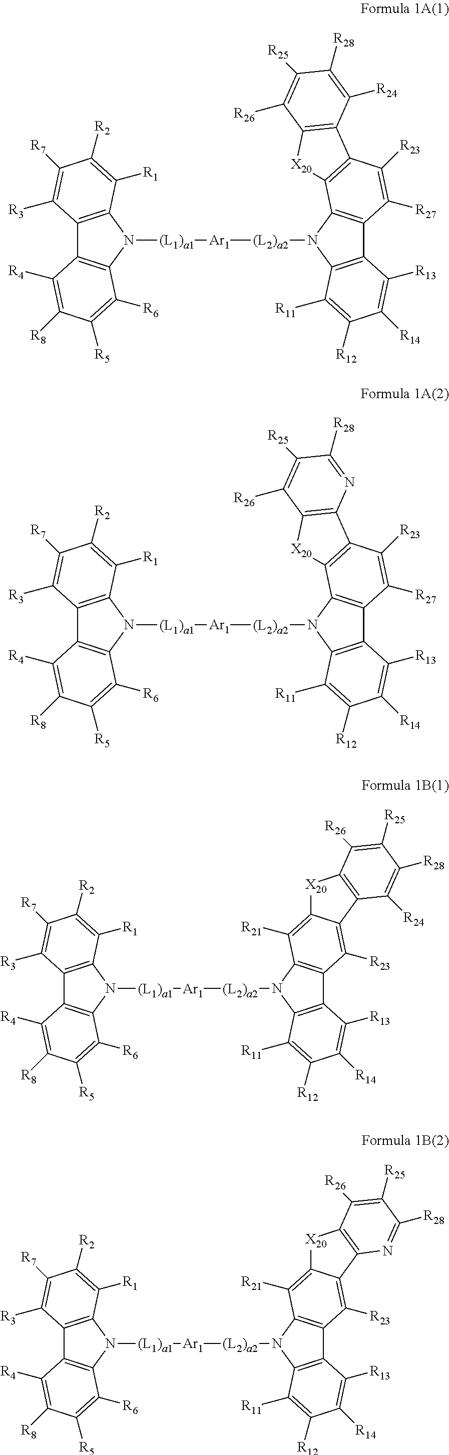 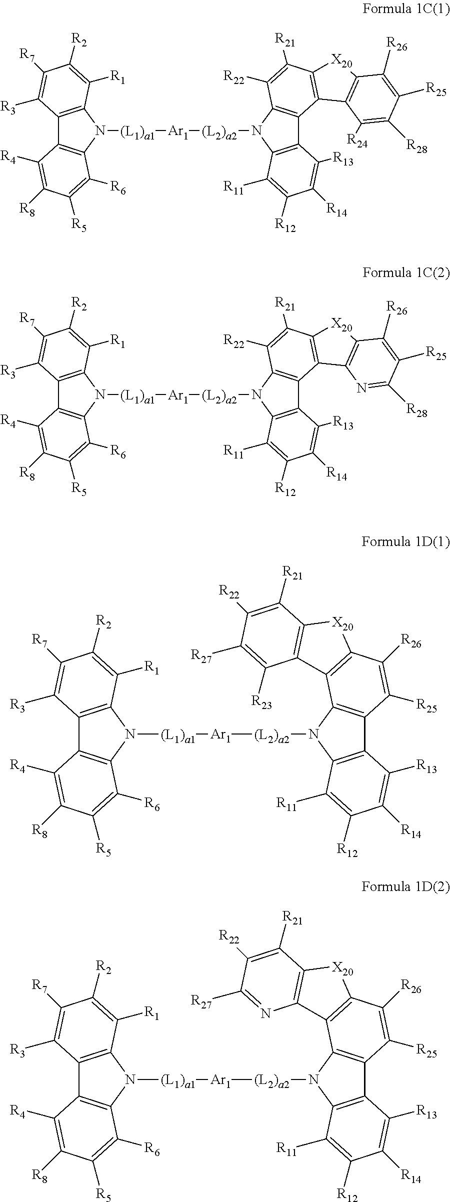 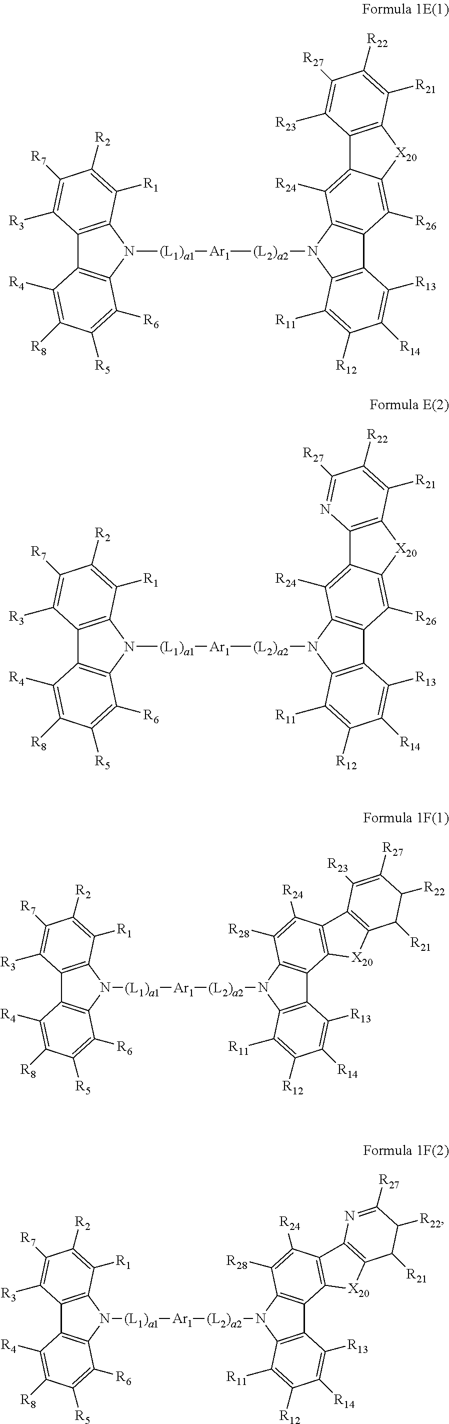
wherein, in Formulae 1A(1), 1A(2), 1B(1), 1B(2), 1C(1), 1C(2), 1D(1), 1D(2), 1E(1), 1E(2), 1F(1), and 1F(2),
Ar₁, L₁, L₂, a1, a2, and X₂₀ are the same as described in claim 1,
R₁ to R₈, R₁₁ to R₁₄, and R₂₀ to R₂₉ are each independently selected from hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a methyl group, an ethyl group, an n-propyl group, an iso-propyl group, an n-butyl group, an iso-butyl group, a sec-butyl group, a tert-butyl group, an n-pentyl group, an iso-pentyl group, a sec-pentyl group, a tert-pentyl group, an n-hexyl group, an iso-hexyl group, a sec-hexyl group, a tert-hexyl group, an n-heptyl group, an iso-heptyl group, a sec-heptyl group, a tert-heptyl group, an n-octyl group, an iso-octyl group, a sec-octyl group, a tert-octyl group, an n-nonyl group, an iso-nonyl group, a sec-nonyl group, a tert-nonyl group, an n-decyl group, an iso-decyl group, a sec-decyl group, a tert-decyl group, a methoxy group, an ethoxy group, a propoxy group, a butoxy group, a pentoxy group, a phenyl group, a naphthyl group, a pyridinyl group, a pyrimidinyl group, a triazinyl group, and —Si(Q₁)(Q₂)(Q₃), and
at least one selected from R₇, R₈, R₁₄, R₂₇, and R₂₈ is a cyano group.

14. The condensed cyclic compound of claim 1, wherein
the condensed cyclic compound is selected from Compounds 1 to 876:

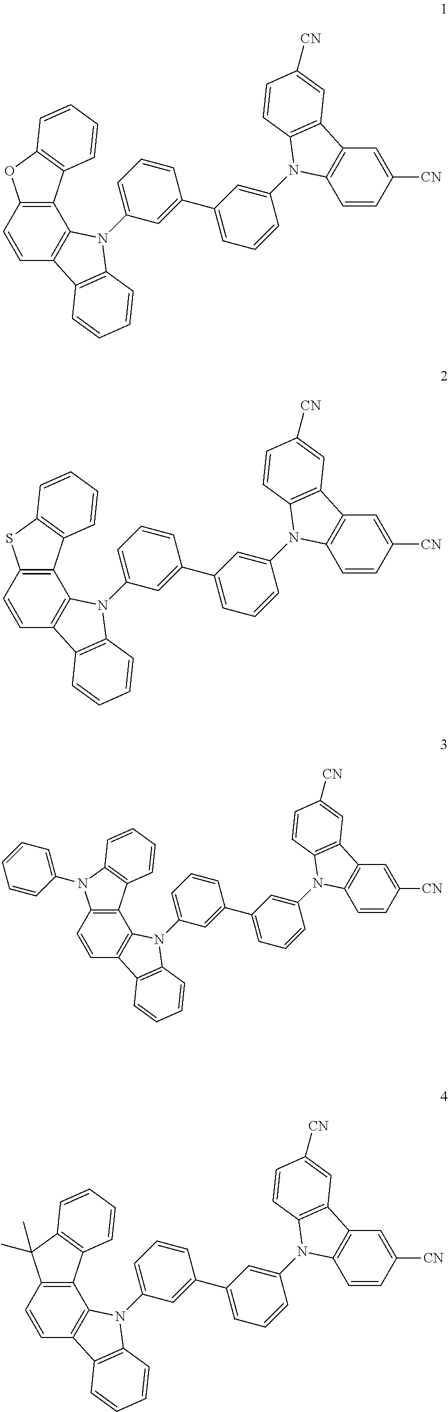 [SMILES: N#Cc1ccc2c(c1)c1cc(C#N)ccc1n2-c1cccc(-c2cccc(-n3c4ccccc4c4ccc5oc6ccccc6c5c43)c2)c1 ; N#Cc1ccc2c(c1)c1cc(C#N)ccc1n2-c1cccc(-c2cccc(-n3c4ccccc4c4ccc5sc6ccccc6c5c43)c2)c1 ; N#Cc1ccc2c(c1)c1cc(C#N)ccc1n2-c1cccc(-c2cccc(-n3c4ccccc4c4ccc5c(c6ccccc6n5-c5ccccc5)c43)c2)c1 ; CC1(C)c2ccccc2-c2c1ccc1c3ccccc3n(-c3cccc(-c4cccc(-n5c6ccc(C#N)cc6c6cc(C#N)ccc65)c4)c3)c21]

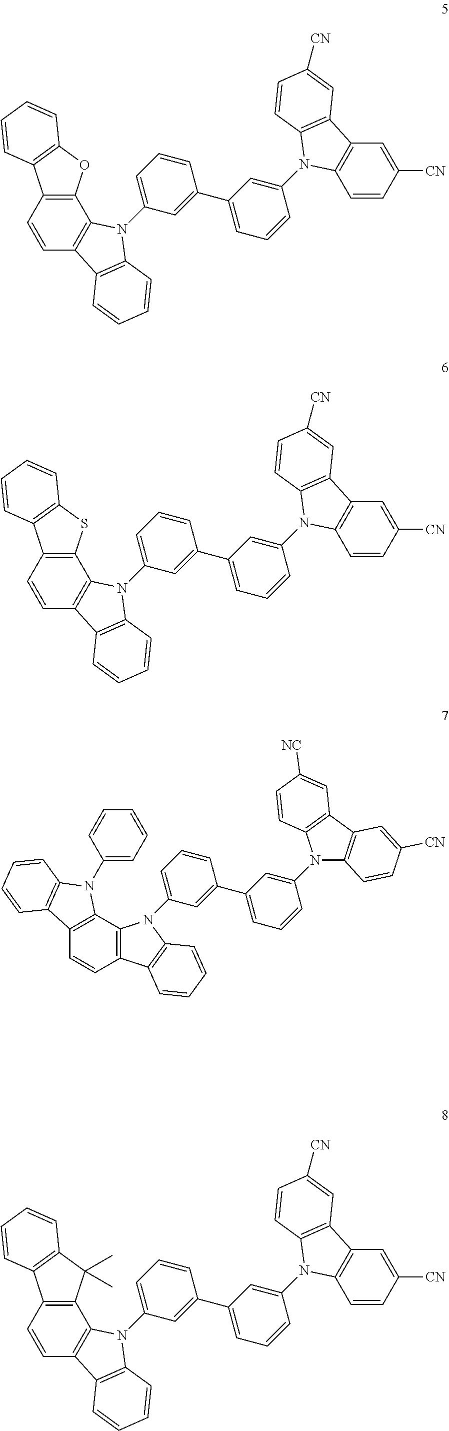 [SMILES: N#Cc1ccc2c(c1)c1cc(C#N)ccc1n2-c1cccc(-c2cccc(-n3c4ccccc4c4ccc5c6ccccc6oc5c43)c2)c1 ; N#Cc1ccc2c(c1)c1cc(C#N)ccc1n2-c1cccc(-c2cccc(-n3c4ccccc4c4ccc5c6ccccc6sc5c43)c2)c1 ; N#Cc1ccc2c(c1)c1cc(C#N)ccc1n2-c1cccc(-c2cccc(-n3c4ccccc4c4ccc5c6ccccc6n(-c6ccccc6)c5c43)c2)c1 ; CC1(C)c2ccccc2-c2ccc3c4ccccc4n(-c4cccc(-c5cccc(-n6c7ccc(C#N)cc7c7cc(C#N)ccc76)c5)c4)c3c21]

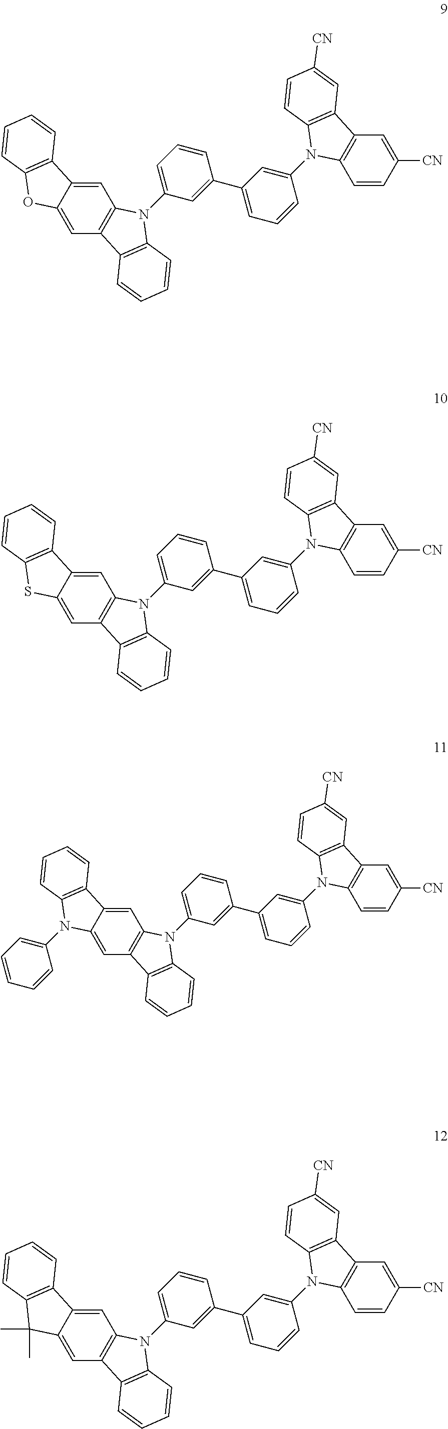 [SMILES: N#Cc1ccc2c(c1)c1cc(C#N)ccc1n2-c1cccc(-c2cccc(-n3c4ccccc4c4cc5oc6ccccc6c5cc43)c2)c1 ; N#Cc1ccc2c(c1)c1cc(C#N)ccc1n2-c1cccc(-c2cccc(-n3c4ccccc4c4cc5sc6ccccc6c5cc43)c2)c1 ; N#Cc1ccc2c(c1)c1cc(C#N)ccc1n2-c1cccc(-c2cccc(-n3c4ccccc4c4cc5c(cc43)c3ccccc3n5-c3ccccc3)c2)c1 ; CC1(C)c2ccccc2-c2cc3c(cc21)c1ccccc1n3-c1cccc(-c2cccc(-n3c4ccc(C#N)cc4c4cc(C#N)ccc43)c2)c1]

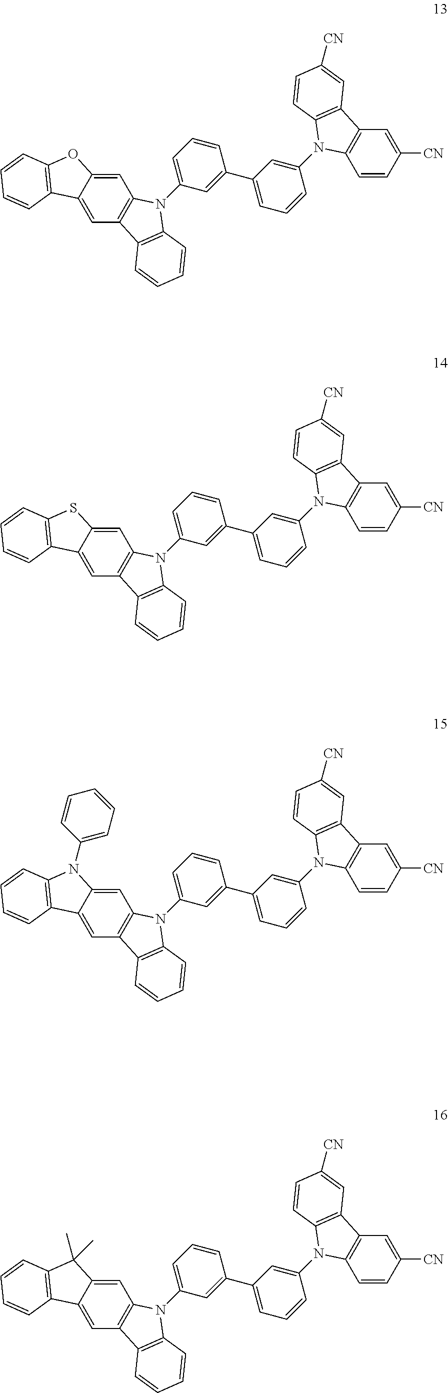 [SMILES: N#Cc1ccc2c(c1)c1cc(C#N)ccc1n2-c1cccc(-c2cccc(-n3c4ccccc4c4cc5c(cc43)oc3ccccc35)c2)c1 ; N#Cc1ccc2c(c1)c1cc(C#N)ccc1n2-c1cccc(-c2cccc(-n3c4ccccc4c4cc5c(cc43)sc3ccccc35)c2)c1 ; N#Cc1ccc2c(c1)c1cc(C#N)ccc1n2-c1cccc(-c2cccc(-n3c4ccccc4c4cc5c6ccccc6n(-c6ccccc6)c5cc43)c2)c1 ; CC1(C)c2ccccc2-c2cc3c4ccccc4n(-c4cccc(-c5cccc(-n6c7ccc(C#N)cc7c7cc(C#N)ccc76)c5)c4)c3cc21]

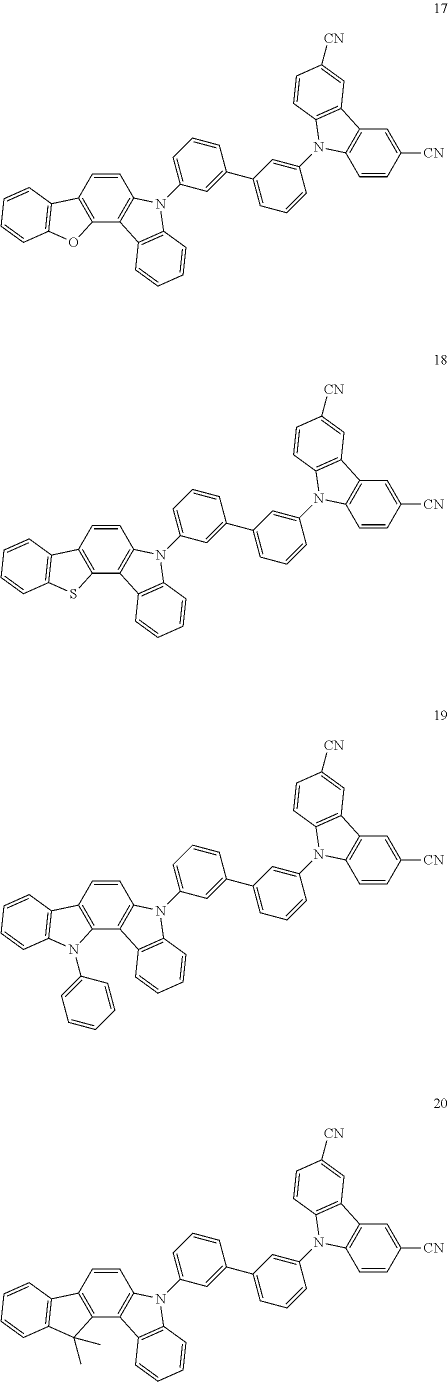 [SMILES: N#Cc1ccc2c(c1)c1cc(C#N)ccc1n2-c1cccc(-c2cccc(-n3c4ccccc4c4c5oc6ccccc6c5ccc43)c2)c1 ; N#Cc1ccc2c(c1)c1cc(C#N)ccc1n2-c1cccc(-c2cccc(-n3c4ccccc4c4c5sc6ccccc6c5ccc43)c2)c1 ; N#Cc1ccc2c(c1)c1cc(C#N)ccc1n2-c1cccc(-c2cccc(-n3c4ccccc4c4c3ccc3c5ccccc5n(-c5ccccc5)c34)c2)c1 ; CC1(C)c2ccccc2-c2ccc3c(c21)c1ccccc1n3-c1cccc(-c2cccc(-n3c4ccc(C#N)cc4c4cc(C#N)ccc43)c2)c1]

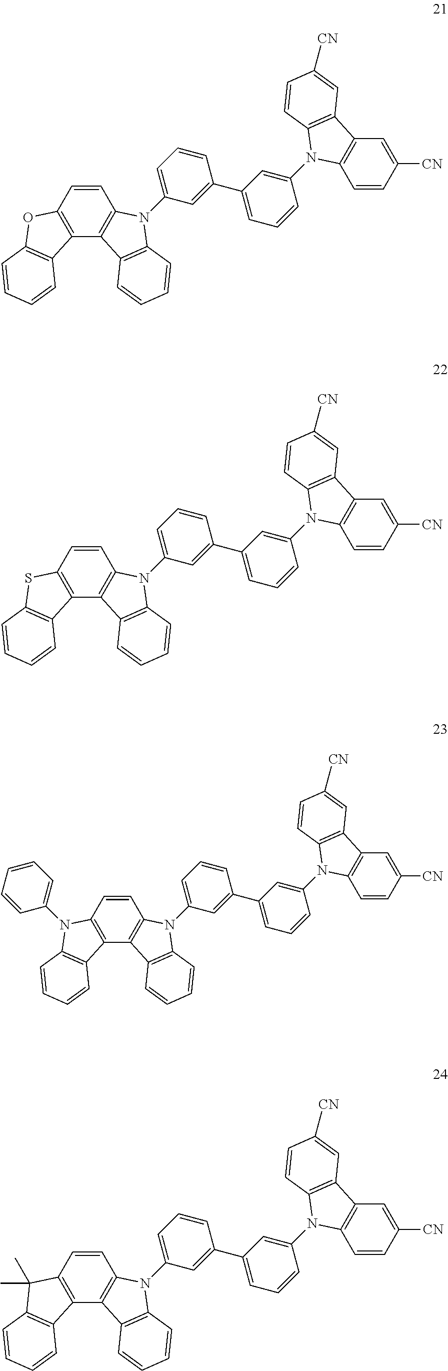 [SMILES: N#Cc1ccc2c(c1)c1cc(C#N)ccc1n2-c1cccc(-c2cccc(-n3c4ccccc4c4c5c(ccc43)oc3ccccc35)c2)c1 ; N#Cc1ccc2c(c1)c1cc(C#N)ccc1n2-c1cccc(-c2cccc(-n3c4ccccc4c4c5c(ccc43)sc3ccccc35)c2)c1 ; N#Cc1ccc2c(c1)c1cc(C#N)ccc1n2-c1cccc(-c2cccc(-n3c4ccccc4c4c5c6ccccc6n(-c6ccccc6)c5ccc43)c2)c1 ; CC1(C)c2ccccc2-c2c1ccc1c2c2ccccc2n1-c1cccc(-c2cccc(-n3c4ccc(C#N)cc4c4cc(C#N)ccc43)c2)c1]

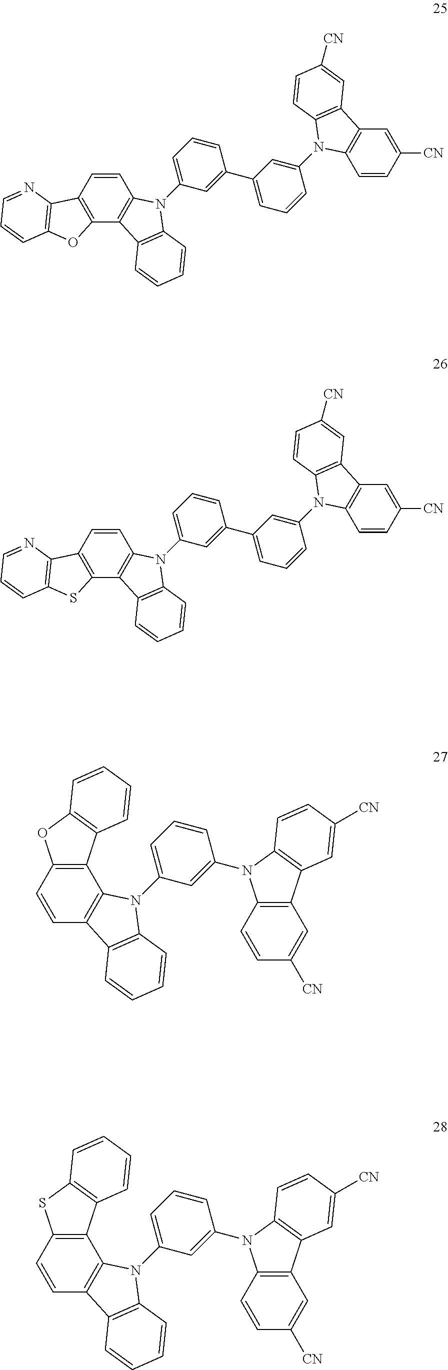 [SMILES: N#Cc1ccc2c(c1)c1cc(C#N)ccc1n2-c1cccc(-c2cccc(-n3c4ccccc4c4c5oc6cccnc6c5ccc43)c2)c1 ; N#Cc1ccc2c(c1)c1cc(C#N)ccc1n2-c1cccc(-c2cccc(-n3c4ccccc4c4c5sc6cccnc6c5ccc43)c2)c1 ; N#Cc1ccc2c(c1)c1cc(C#N)ccc1n2-c1cccc(-n2c3ccccc3c3ccc4oc5ccccc5c4c32)c1 ; N#Cc1ccc2c(c1)c1cc(C#N)ccc1n2-c1cccc(-n2c3ccccc3c3ccc4sc5ccccc5c4c32)c1]

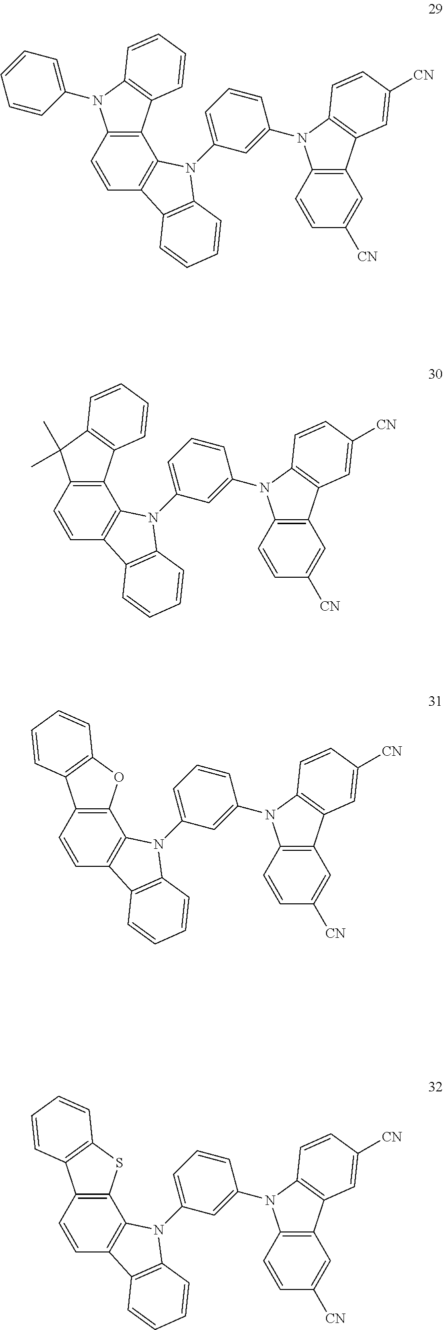 [SMILES: N#Cc1ccc2c(c1)c1cc(C#N)ccc1n2-c1cccc(-n2c3ccccc3c3ccc4c(c5ccccc5n4-c4ccccc4)c32)c1 ; CC1(C)c2ccccc2-c2c1ccc1c3ccccc3n(-c3cccc(-n4c5ccc(C#N)cc5c5cc(C#N)ccc54)c3)c21 ; N#Cc1ccc2c(c1)c1cc(C#N)ccc1n2-c1cccc(-n2c3ccccc3c3ccc4c5ccccc5oc4c32)c1 ; N#Cc1ccc2c(c1)c1cc(C#N)ccc1n2-c1cccc(-n2c3ccccc3c3ccc4c5ccccc5sc4c32)c1]

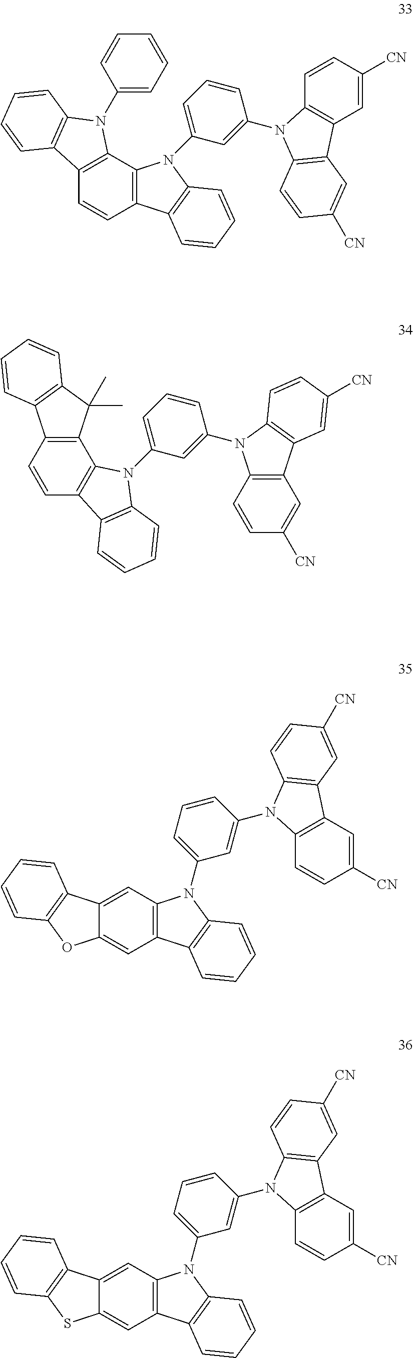 [SMILES: N#Cc1ccc2c(c1)c1cc(C#N)ccc1n2-c1cccc(-n2c3ccccc3c3ccc4c5ccccc5n(-c5ccccc5)c4c32)c1 ; CC1(C)c2ccccc2-c2ccc3c4ccccc4n(-c4cccc(-n5c6ccc(C#N)cc6c6cc(C#N)ccc65)c4)c3c21 ; N#Cc1ccc2c(c1)c1cc(C#N)ccc1n2-c1cccc(-n2c3ccccc3c3cc4oc5ccccc5c4cc32)c1 ; N#Cc1ccc2c(c1)c1cc(C#N)ccc1n2-c1cccc(-n2c3ccccc3c3cc4sc5ccccc5c4cc32)c1]

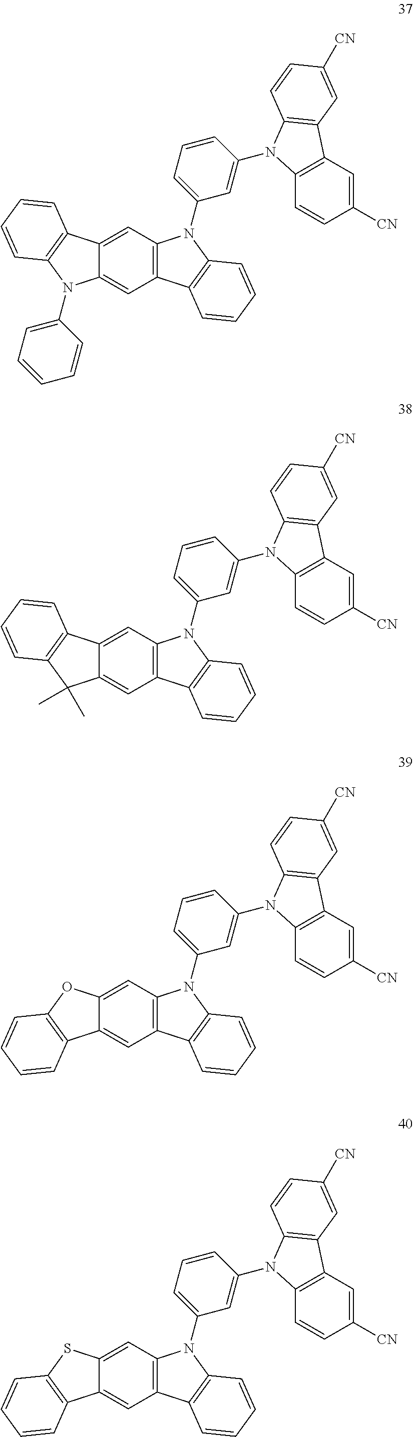 [SMILES: N#Cc1ccc2c(c1)c1cc(C#N)ccc1n2-c1cccc(-n2c3ccccc3c3cc4c(cc32)c2ccccc2n4-c2ccccc2)c1 ; CC1(C)c2ccccc2-c2cc3c(cc21)c1ccccc1n3-c1cccc(-n2c3ccc(C#N)cc3c3cc(C#N)ccc32)c1 ; N#Cc1ccc2c(c1)c1cc(C#N)ccc1n2-c1cccc(-n2c3ccccc3c3cc4c(cc32)oc2ccccc24)c1 ; N#Cc1ccc2c(c1)c1cc(C#N)ccc1n2-c1cccc(-n2c3ccccc3c3cc4c(cc32)sc2ccccc24)c1]

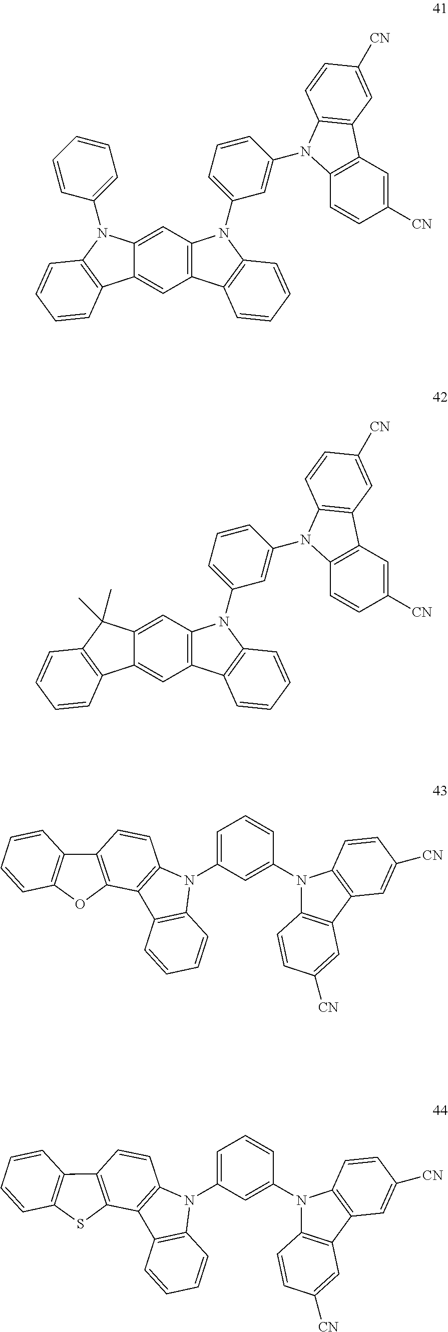 [SMILES: N#Cc1ccc2c(c1)c1cc(C#N)ccc1n2-c1cccc(-n2c3ccccc3c3cc4c5ccccc5n(-c5ccccc5)c4cc32)c1 ; CC1(C)c2ccccc2-c2cc3c4ccccc4n(-c4cccc(-n5c6ccc(C#N)cc6c6cc(C#N)ccc65)c4)c3cc21 ; N#Cc1ccc2c(c1)c1cc(C#N)ccc1n2-c1cccc(-n2c3ccccc3c3c4oc5ccccc5c4ccc32)c1 ; N#Cc1ccc2c(c1)c1cc(C#N)ccc1n2-c1cccc(-n2c3ccccc3c3c4sc5ccccc5c4ccc32)c1]

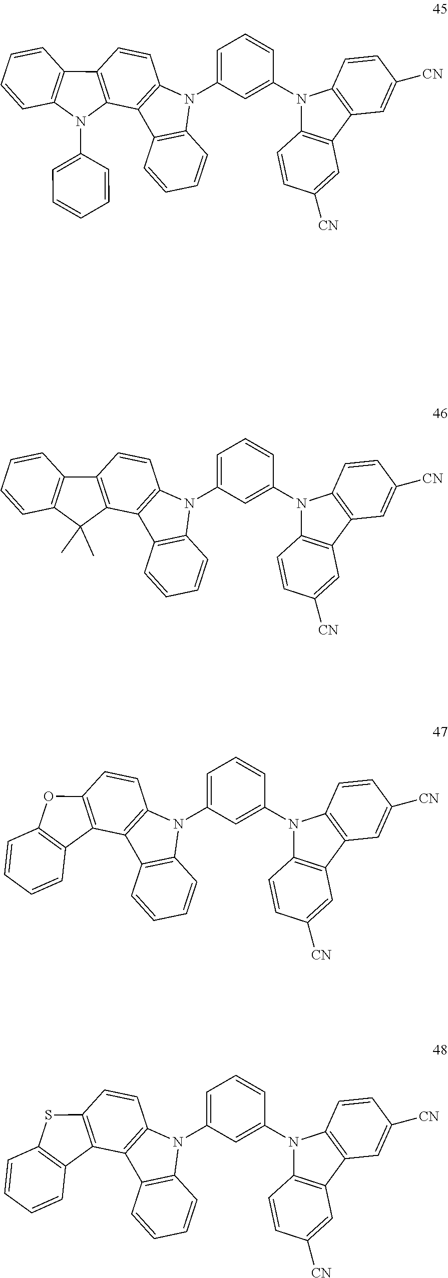 [SMILES: N#Cc1ccc2c(c1)c1cc(C#N)ccc1n2-c1cccc(-n2c3ccccc3c3c2ccc2c4ccccc4n(-c4ccccc4)c23)c1 ; CC1(C)c2ccccc2-c2ccc3c(c21)c1ccccc1n3-c1cccc(-n2c3ccc(C#N)cc3c3cc(C#N)ccc32)c1 ; N#Cc1ccc2c(c1)c1cc(C#N)ccc1n2-c1cccc(-n2c3ccccc3c3c4c(ccc32)oc2ccccc24)c1 ; N#Cc1ccc2c(c1)c1cc(C#N)ccc1n2-c1cccc(-n2c3ccccc3c3c4c(ccc32)sc2ccccc24)c1]

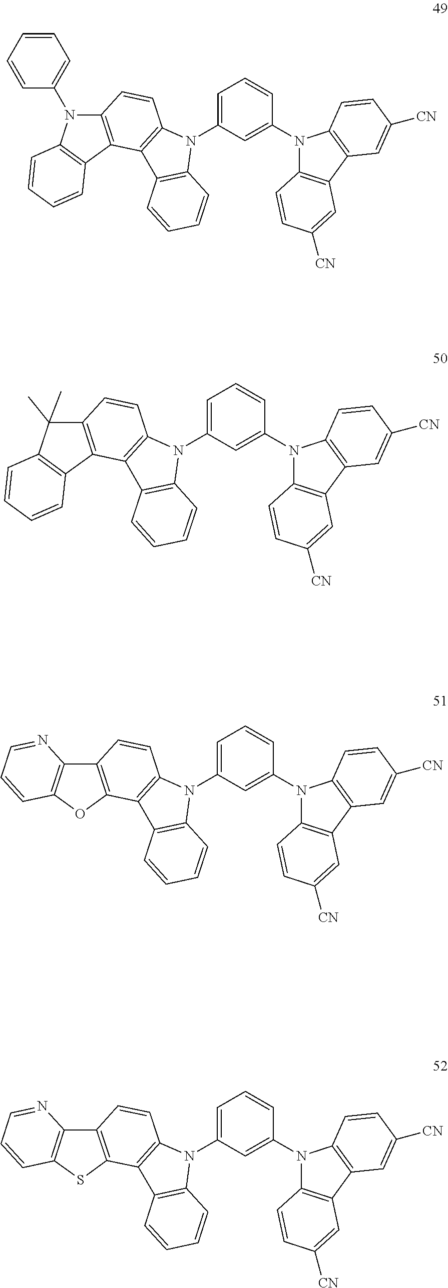 [SMILES: N#Cc1ccc2c(c1)c1cc(C#N)ccc1n2-c1cccc(-n2c3ccccc3c3c4c5ccccc5n(-c5ccccc5)c4ccc32)c1 ; CC1(C)c2ccccc2-c2c1ccc1c2c2ccccc2n1-c1cccc(-n2c3ccc(C#N)cc3c3cc(C#N)ccc32)c1 ; N#Cc1ccc2c(c1)c1cc(C#N)ccc1n2-c1cccc(-n2c3ccccc3c3c4oc5cccnc5c4ccc32)c1 ; N#Cc1ccc2c(c1)c1cc(C#N)ccc1n2-c1cccc(-n2c3ccccc3c3c4sc5cccnc5c4ccc32)c1]

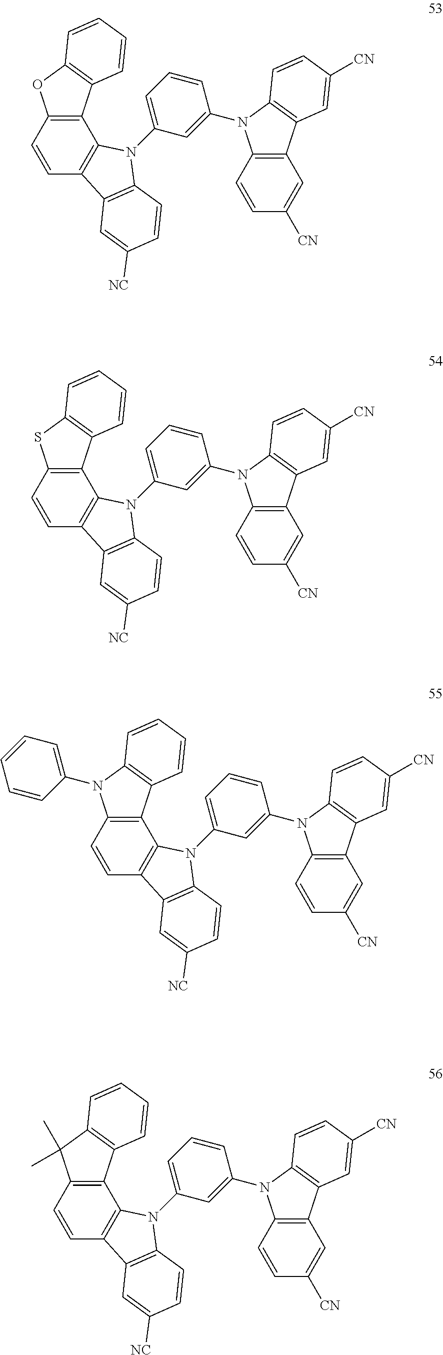 [SMILES: N#Cc1ccc2c(c1)c1cc(C#N)ccc1n2-c1cccc(-n2c3ccc(C#N)cc3c3ccc4oc5ccccc5c4c32)c1 ; N#Cc1ccc2c(c1)c1cc(C#N)ccc1n2-c1cccc(-n2c3ccc(C#N)cc3c3ccc4sc5ccccc5c4c32)c1 ; N#Cc1ccc2c(c1)c1cc(C#N)ccc1n2-c1cccc(-n2c3ccc(C#N)cc3c3ccc4c(c5ccccc5n4-c4ccccc4)c32)c1 ; CC1(C)c2ccccc2-c2c1ccc1c3cc(C#N)ccc3n(-c3cccc(-n4c5ccc(C#N)cc5c5cc(C#N)ccc54)c3)c21]

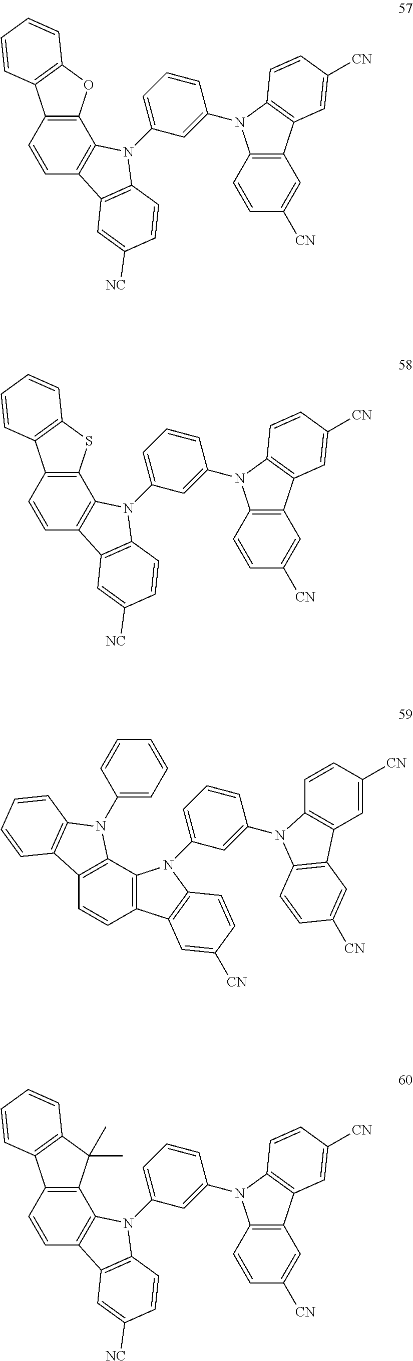 [SMILES: N#Cc1ccc2c(c1)c1cc(C#N)ccc1n2-c1cccc(-n2c3ccc(C#N)cc3c3ccc4c5ccccc5oc4c32)c1 ; N#Cc1ccc2c(c1)c1cc(C#N)ccc1n2-c1cccc(-n2c3ccc(C#N)cc3c3ccc4c5ccccc5sc4c32)c1 ; N#Cc1ccc2c(c1)c1cc(C#N)ccc1n2-c1cccc(-n2c3ccc(C#N)cc3c3ccc4c5ccccc5n(-c5ccccc5)c4c32)c1 ; CC1(C)c2ccccc2-c2ccc3c4cc(C#N)ccc4n(-c4cccc(-n5c6ccc(C#N)cc6c6cc(C#N)ccc65)c4)c3c21]

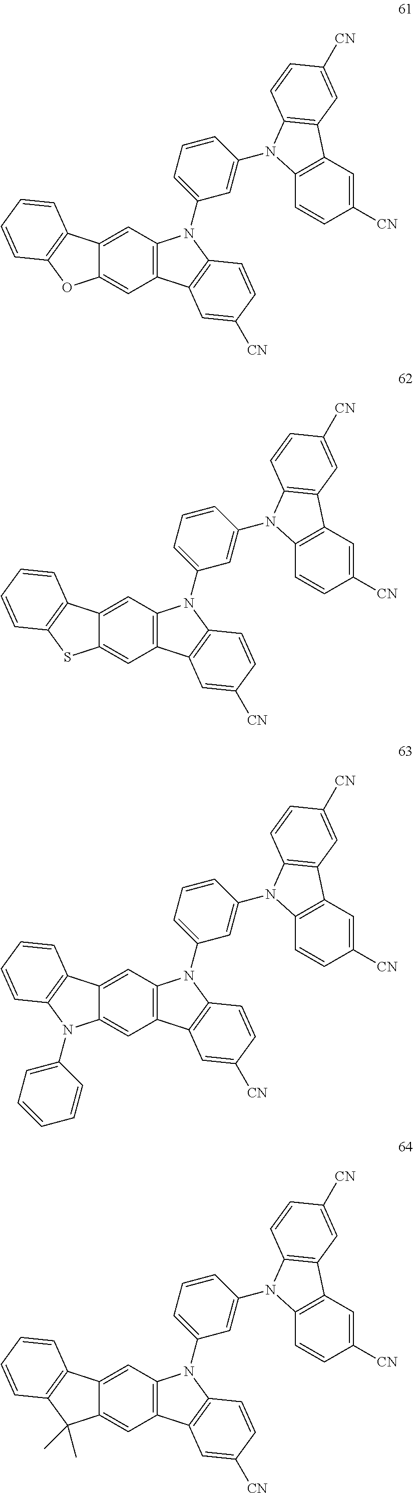 [SMILES: N#Cc1ccc2c(c1)c1cc(C#N)ccc1n2-c1cccc(-n2c3ccc(C#N)cc3c3cc4oc5ccccc5c4cc32)c1 ; N#Cc1ccc2c(c1)c1cc(C#N)ccc1n2-c1cccc(-n2c3ccc(C#N)cc3c3cc4sc5ccccc5c4cc32)c1 ; N#Cc1ccc2c(c1)c1cc(C#N)ccc1n2-c1cccc(-n2c3ccc(C#N)cc3c3cc4c(cc32)c2ccccc2n4-c2ccccc2)c1 ; CC1(C)c2ccccc2-c2cc3c(cc21)c1cc(C#N)ccc1n3-c1cccc(-n2c3ccc(C#N)cc3c3cc(C#N)ccc32)c1]

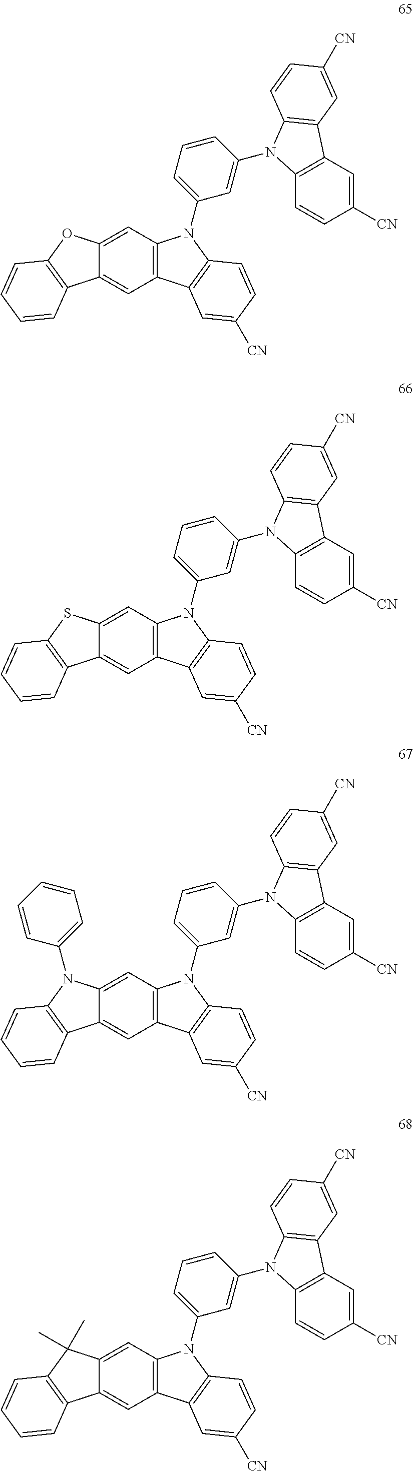 [SMILES: N#Cc1ccc2c(c1)c1cc(C#N)ccc1n2-c1cccc(-n2c3ccc(C#N)cc3c3cc4c(cc32)oc2ccccc24)c1 ; N#Cc1ccc2c(c1)c1cc(C#N)ccc1n2-c1cccc(-n2c3ccc(C#N)cc3c3cc4c(cc32)sc2ccccc24)c1 ; N#Cc1ccc2c(c1)c1cc(C#N)ccc1n2-c1cccc(-n2c3ccc(C#N)cc3c3cc4c5ccccc5n(-c5ccccc5)c4cc32)c1 ; CC1(C)c2ccccc2-c2cc3c4cc(C#N)ccc4n(-c4cccc(-n5c6ccc(C#N)cc6c6cc(C#N)ccc65)c4)c3cc21]

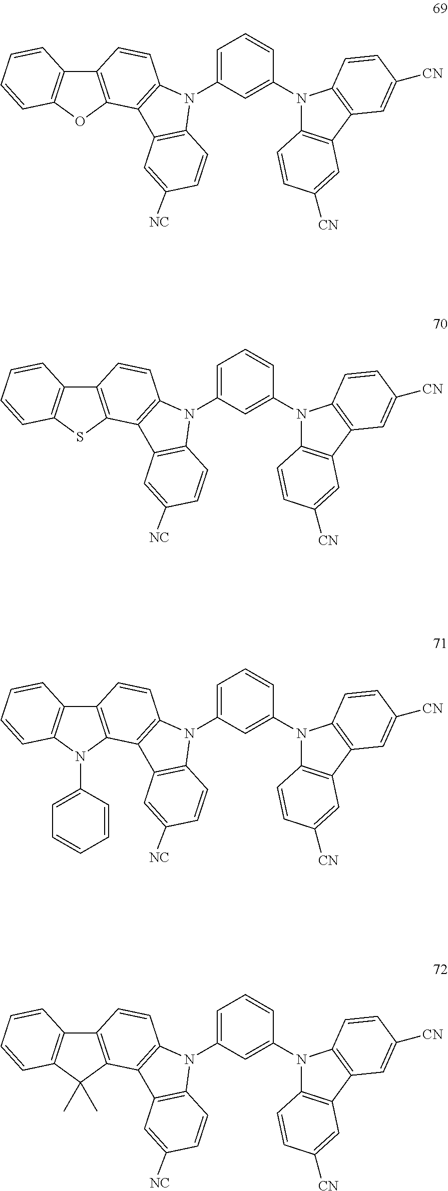

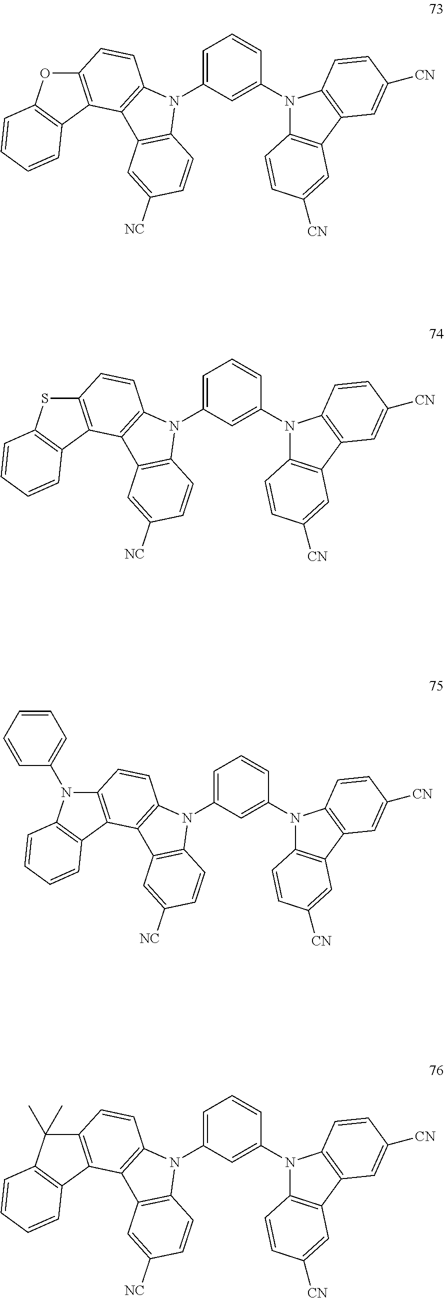 [SMILES: N#Cc1ccc2c(c1)c1cc(C#N)ccc1n2-c1cccc(-n2c3ccc(C#N)cc3c3c4c(ccc32)oc2ccccc24)c1 ; N#Cc1ccc2c(c1)c1cc(C#N)ccc1n2-c1cccc(-n2c3ccc(C#N)cc3c3c4c(ccc32)sc2ccccc24)c1 ; N#Cc1ccc2c(c1)c1cc(C#N)ccc1n2-c1cccc(-n2c3ccc(C#N)cc3c3c4c5ccccc5n(-c5ccccc5)c4ccc32)c1 ; CC1(C)c2ccccc2-c2c1ccc1c2c2cc(C#N)ccc2n1-c1cccc(-n2c3ccc(C#N)cc3c3cc(C#N)ccc32)c1]

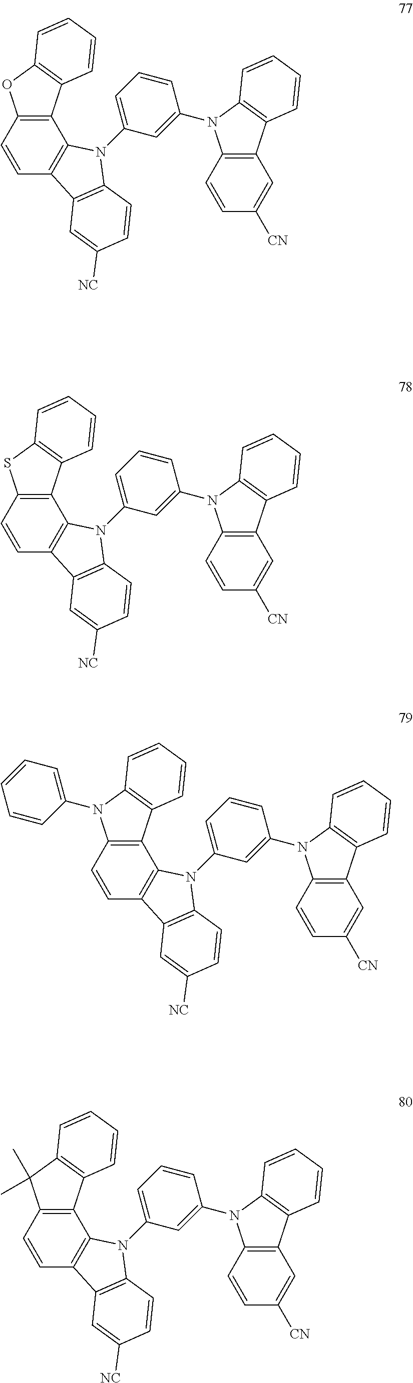 [SMILES: N#Cc1ccc2c(c1)c1ccccc1n2-c1cccc(-n2c3ccc(C#N)cc3c3ccc4oc5ccccc5c4c32)c1 ; N#Cc1ccc2c(c1)c1ccccc1n2-c1cccc(-n2c3ccc(C#N)cc3c3ccc4sc5ccccc5c4c32)c1 ; N#Cc1ccc2c(c1)c1ccccc1n2-c1cccc(-n2c3ccc(C#N)cc3c3ccc4c(c5ccccc5n4-c4ccccc4)c32)c1 ; CC1(C)c2ccccc2-c2c1ccc1c3cc(C#N)ccc3n(-c3cccc(-n4c5ccccc5c5cc(C#N)ccc54)c3)c21]

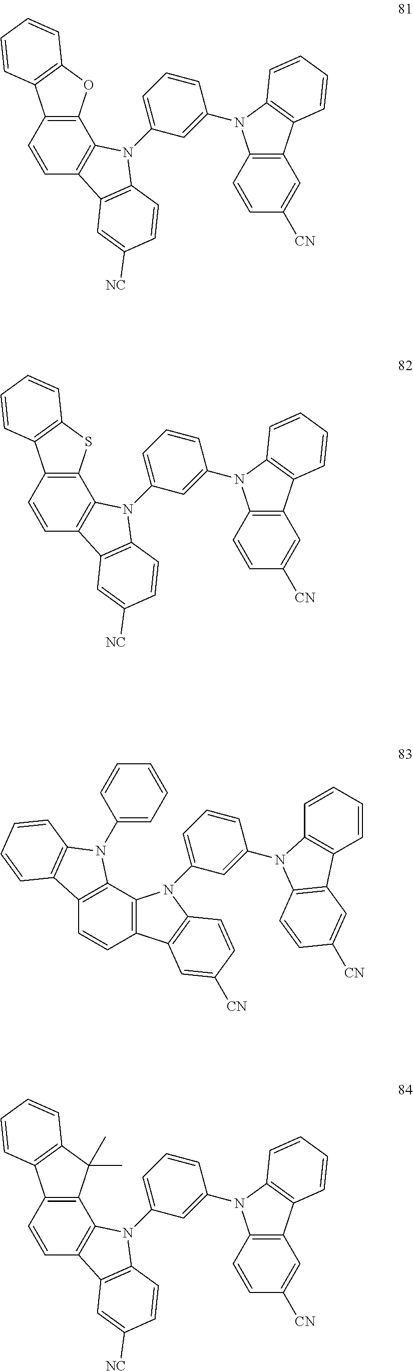 [SMILES: N#Cc1ccc2c(c1)c1ccccc1n2-c1cccc(-n2c3ccc(C#N)cc3c3ccc4c5ccccc5oc4c32)c1 ; N#Cc1ccc2c(c1)c1ccccc1n2-c1cccc(-n2c3ccc(C#N)cc3c3ccc4c5ccccc5sc4c32)c1 ; N#Cc1ccc2c(c1)c1ccccc1n2-c1cccc(-n2c3ccc(C#N)cc3c3ccc4c5ccccc5n(-c5ccccc5)c4c32)c1 ; CC1(C)c2ccccc2-c2ccc3c4cc(C#N)ccc4n(-c4cccc(-n5c6ccccc6c6cc(C#N)ccc65)c4)c3c21]

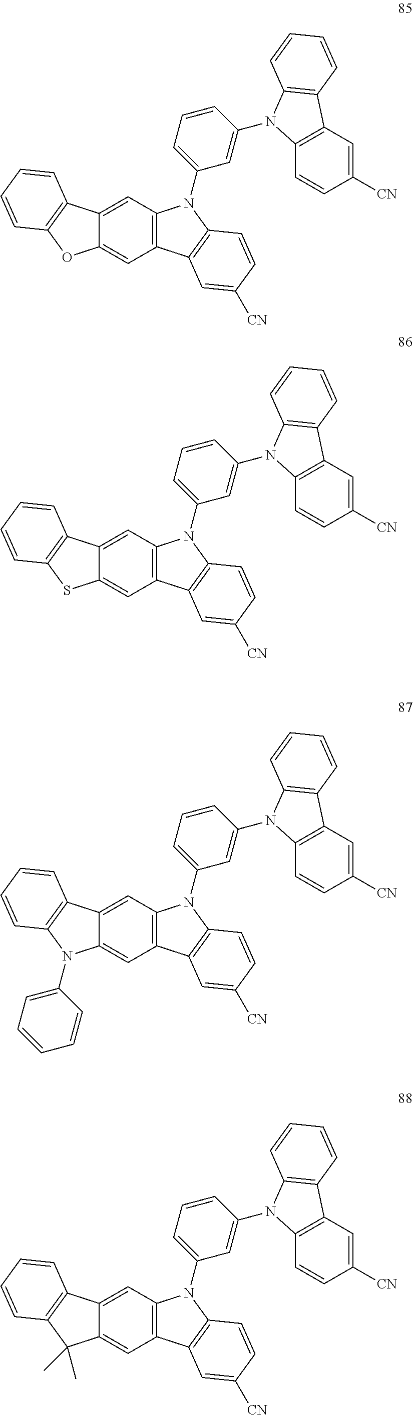 [SMILES: N#Cc1ccc2c(c1)c1ccccc1n2-c1cccc(-n2c3ccc(C#N)cc3c3cc4oc5ccccc5c4cc32)c1 ; N#Cc1ccc2c(c1)c1ccccc1n2-c1cccc(-n2c3ccc(C#N)cc3c3cc4sc5ccccc5c4cc32)c1 ; N#Cc1ccc2c(c1)c1ccccc1n2-c1cccc(-n2c3ccc(C#N)cc3c3cc4c(cc32)c2ccccc2n4-c2ccccc2)c1 ; CC1(C)c2ccccc2-c2cc3c(cc21)c1cc(C#N)ccc1n3-c1cccc(-n2c3ccccc3c3cc(C#N)ccc32)c1]

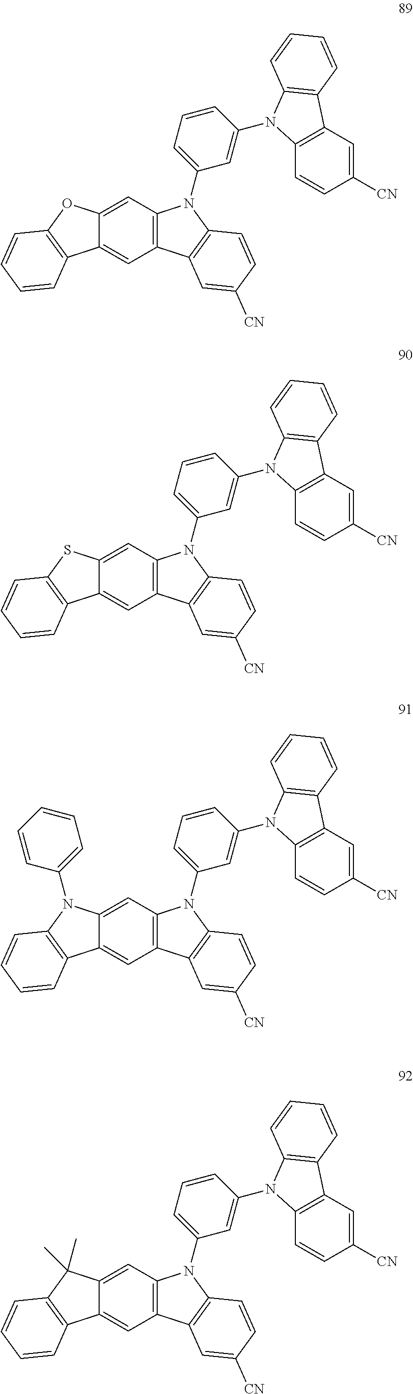 [SMILES: N#Cc1ccc2c(c1)c1ccccc1n2-c1cccc(-n2c3ccc(C#N)cc3c3cc4c(cc32)oc2ccccc24)c1 ; N#Cc1ccc2c(c1)c1ccccc1n2-c1cccc(-n2c3ccc(C#N)cc3c3cc4c(cc32)sc2ccccc24)c1 ; N#Cc1ccc2c(c1)c1ccccc1n2-c1cccc(-n2c3ccc(C#N)cc3c3cc4c5ccccc5n(-c5ccccc5)c4cc32)c1 ; CC1(C)c2ccccc2-c2cc3c4cc(C#N)ccc4n(-c4cccc(-n5c6ccccc6c6cc(C#N)ccc65)c4)c3cc21]

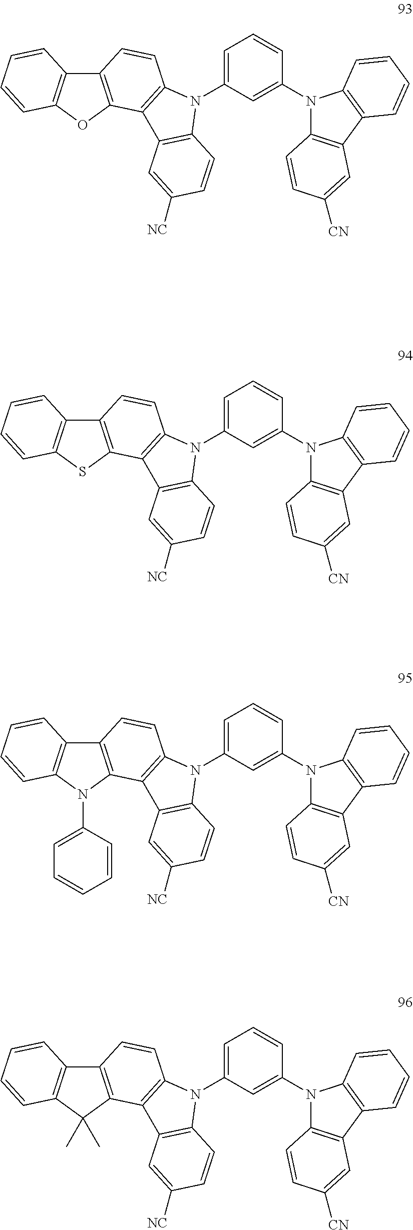 [SMILES: N#Cc1ccc2c(c1)c1ccccc1n2-c1cccc(-n2c3ccc(C#N)cc3c3c4oc5ccccc5c4ccc32)c1 ; N#Cc1ccc2c(c1)c1ccccc1n2-c1cccc(-n2c3ccc(C#N)cc3c3c4sc5ccccc5c4ccc32)c1 ; N#Cc1ccc2c(c1)c1ccccc1n2-c1cccc(-n2c3ccc(C#N)cc3c3c2ccc2c4ccccc4n(-c4ccccc4)c23)c1 ; CC1(C)c2ccccc2-c2ccc3c(c21)c1cc(C#N)ccc1n3-c1cccc(-n2c3ccccc3c3cc(C#N)ccc32)c1]

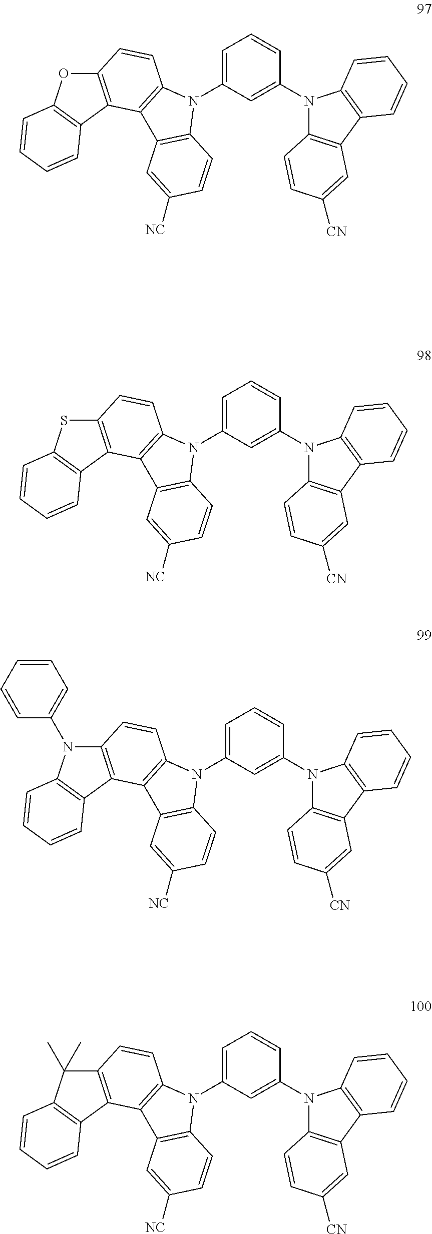 [SMILES: N#Cc1ccc2c(c1)c1ccccc1n2-c1cccc(-n2c3ccc(C#N)cc3c3c4c(ccc32)oc2ccccc24)c1 ; N#Cc1ccc2c(c1)c1ccccc1n2-c1cccc(-n2c3ccc(C#N)cc3c3c4c(ccc32)sc2ccccc24)c1 ; N#Cc1ccc2c(c1)c1ccccc1n2-c1cccc(-n2c3ccc(C#N)cc3c3c4c5ccccc5n(-c5ccccc5)c4ccc32)c1 ; CC1(C)c2ccccc2-c2c1ccc1c2c2cc(C#N)ccc2n1-c1cccc(-n2c3ccccc3c3cc(C#N)ccc32)c1]

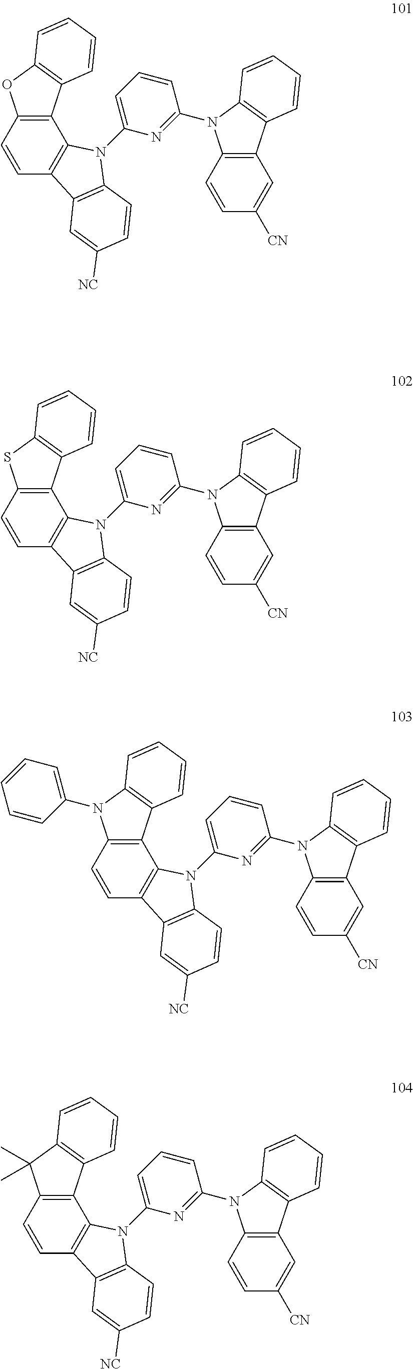 [SMILES: N#Cc1ccc2c(c1)c1ccccc1n2-c1cccc(-n2c3ccc(C#N)cc3c3ccc4oc5ccccc5c4c32)n1 ; N#Cc1ccc2c(c1)c1ccccc1n2-c1cccc(-n2c3ccc(C#N)cc3c3ccc4sc5ccccc5c4c32)n1 ; N#Cc1ccc2c(c1)c1ccccc1n2-c1cccc(-n2c3ccc(C#N)cc3c3ccc4c(c5ccccc5n4-c4ccccc4)c32)n1 ; CC1(C)c2ccccc2-c2c1ccc1c3cc(C#N)ccc3n(-c3cccc(-n4c5ccccc5c5cc(C#N)ccc54)n3)c21]

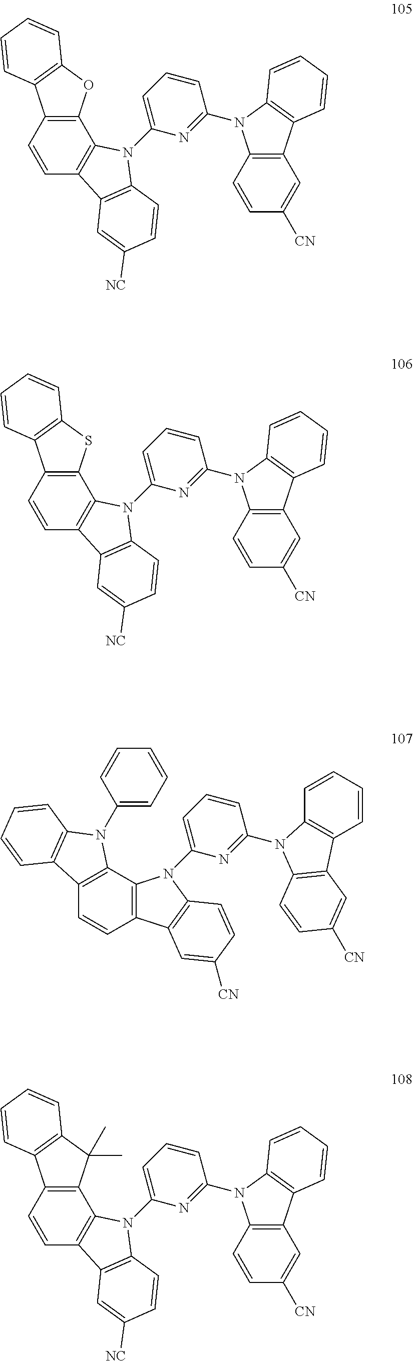 [SMILES: N#Cc1ccc2c(c1)c1ccccc1n2-c1cccc(-n2c3ccc(C#N)cc3c3ccc4c5ccccc5oc4c32)n1 ; N#Cc1ccc2c(c1)c1ccccc1n2-c1cccc(-n2c3ccc(C#N)cc3c3ccc4c5ccccc5sc4c32)n1 ; N#Cc1ccc2c(c1)c1ccccc1n2-c1cccc(-n2c3ccc(C#N)cc3c3ccc4c5ccccc5n(-c5ccccc5)c4c32)n1 ; CC1(C)c2ccccc2-c2ccc3c4cc(C#N)ccc4n(-c4cccc(-n5c6ccccc6c6cc(C#N)ccc65)n4)c3c21]

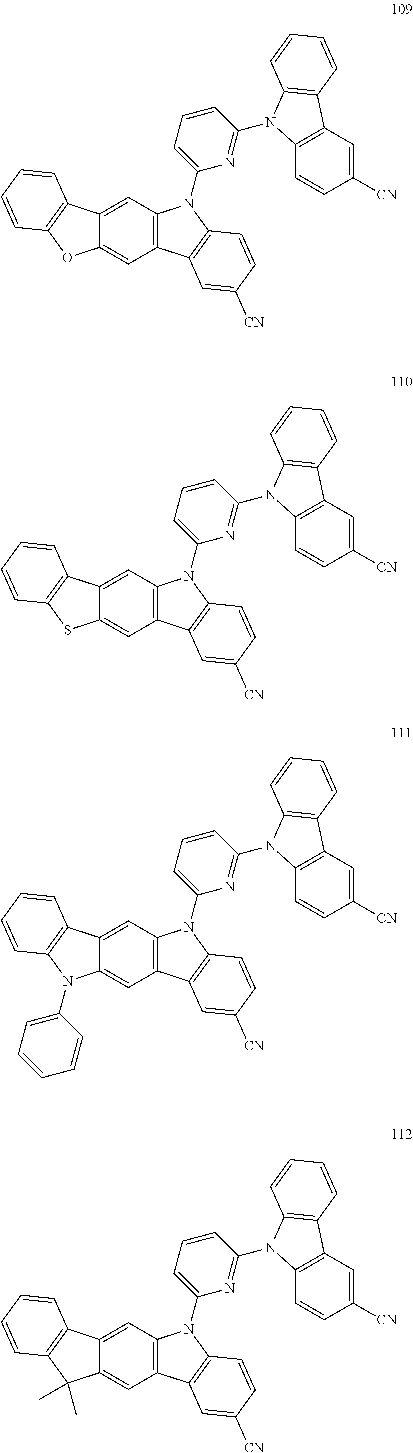 [SMILES: N#Cc1ccc2c(c1)c1ccccc1n2-c1cccc(-n2c3ccc(C#N)cc3c3cc4oc5ccccc5c4cc32)n1 ; N#Cc1ccc2c(c1)c1ccccc1n2-c1cccc(-n2c3ccc(C#N)cc3c3cc4sc5ccccc5c4cc32)n1 ; N#Cc1ccc2c(c1)c1ccccc1n2-c1cccc(-n2c3ccc(C#N)cc3c3cc4c(cc32)c2ccccc2n4-c2ccccc2)n1 ; CC1(C)c2ccccc2-c2cc3c(cc21)c1cc(C#N)ccc1n3-c1cccc(-n2c3ccccc3c3cc(C#N)ccc32)n1]

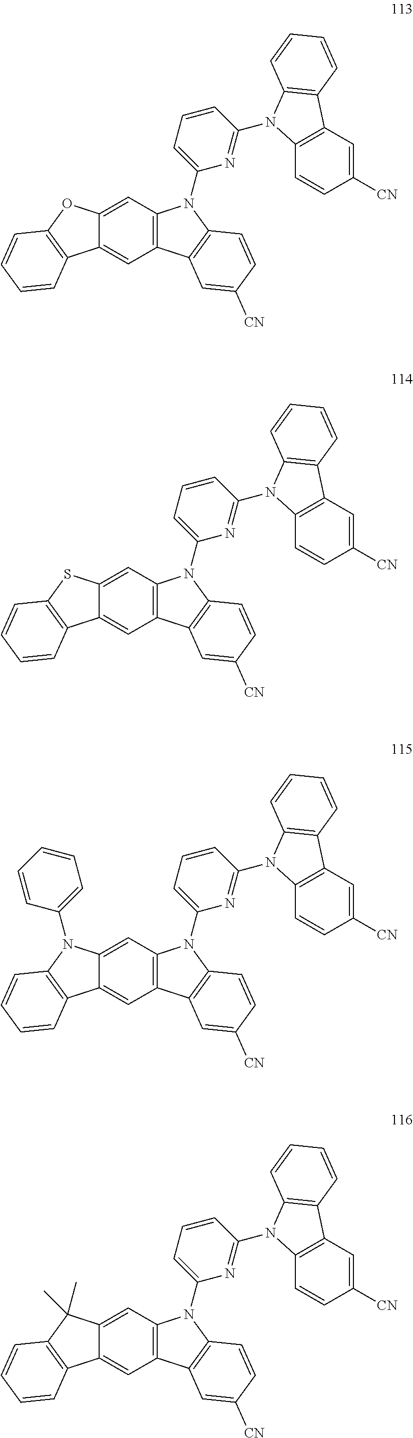 [SMILES: N#Cc1ccc2c(c1)c1ccccc1n2-c1cccc(-n2c3ccc(C#N)cc3c3cc4c(cc32)oc2ccccc24)n1 ; N#Cc1ccc2c(c1)c1ccccc1n2-c1cccc(-n2c3ccc(C#N)cc3c3cc4c(cc32)sc2ccccc24)n1 ; N#Cc1ccc2c(c1)c1ccccc1n2-c1cccc(-n2c3ccc(C#N)cc3c3cc4c5ccccc5n(-c5ccccc5)c4cc32)n1 ; CC1(C)c2ccccc2-c2cc3c4cc(C#N)ccc4n(-c4cccc(-n5c6ccccc6c6cc(C#N)ccc65)n4)c3cc21]

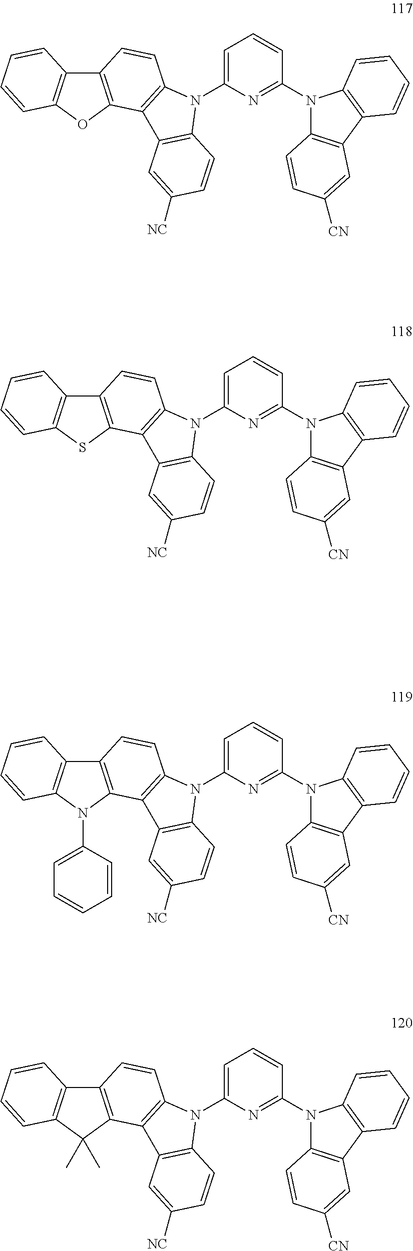 [SMILES: N#Cc1ccc2c(c1)c1ccccc1n2-c1cccc(-n2c3ccc(C#N)cc3c3c4oc5ccccc5c4ccc32)n1 ; N#Cc1ccc2c(c1)c1ccccc1n2-c1cccc(-n2c3ccc(C#N)cc3c3c4sc5ccccc5c4ccc32)n1 ; N#Cc1ccc2c(c1)c1ccccc1n2-c1cccc(-n2c3ccc(C#N)cc3c3c2ccc2c4ccccc4n(-c4ccccc4)c23)n1 ; CC1(C)c2ccccc2-c2ccc3c(c21)c1cc(C#N)ccc1n3-c1cccc(-n2c3ccccc3c3cc(C#N)ccc32)n1]

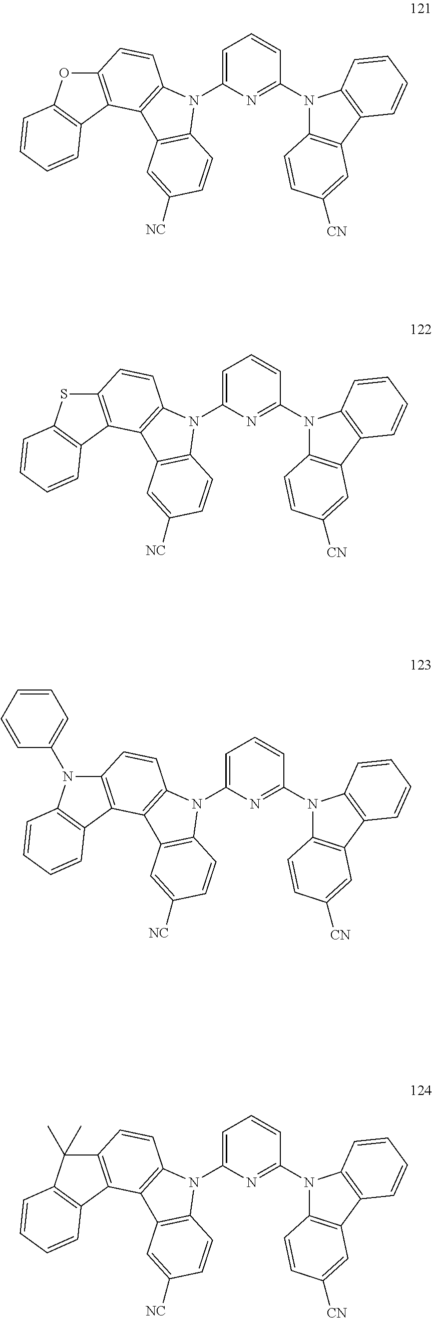 [SMILES: N#Cc1ccc2c(c1)c1ccccc1n2-c1cccc(-n2c3ccc(C#N)cc3c3c4c(ccc32)oc2ccccc24)n1 ; N#Cc1ccc2c(c1)c1ccccc1n2-c1cccc(-n2c3ccc(C#N)cc3c3c4c(ccc32)sc2ccccc24)n1 ; N#Cc1ccc2c(c1)c1ccccc1n2-c1cccc(-n2c3ccc(C#N)cc3c3c4c5ccccc5n(-c5ccccc5)c4ccc32)n1 ; CC1(C)c2ccccc2-c2c1ccc1c2c2cc(C#N)ccc2n1-c1cccc(-n2c3ccccc3c3cc(C#N)ccc32)n1]

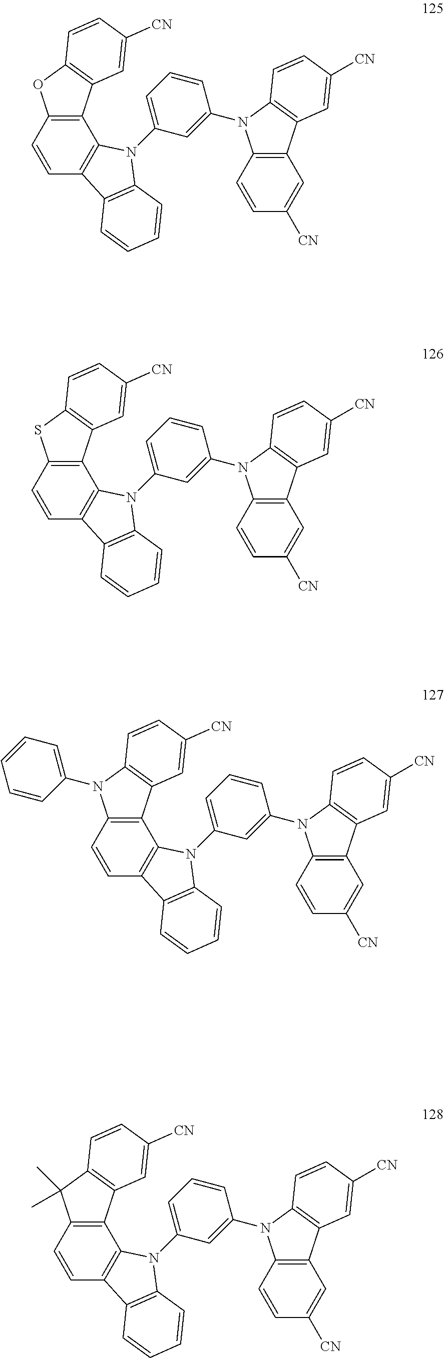 [SMILES: N#Cc1ccc2oc3ccc4c5ccccc5n(-c5cccc(-n6c7ccc(C#N)cc7c7cc(C#N)ccc76)c5)c4c3c2c1 ; N#Cc1ccc2sc3ccc4c5ccccc5n(-c5cccc(-n6c7ccc(C#N)cc7c7cc(C#N)ccc76)c5)c4c3c2c1 ; N#Cc1ccc2c(c1)c1cc(C#N)ccc1n2-c1cccc(-n2c3ccccc3c3ccc4c(c5cc(C#N)ccc5n4-c4ccccc4)c32)c1 ; CC1(C)c2ccc(C#N)cc2-c2c1ccc1c3ccccc3n(-c3cccc(-n4c5ccc(C#N)cc5c5cc(C#N)ccc54)c3)c21]

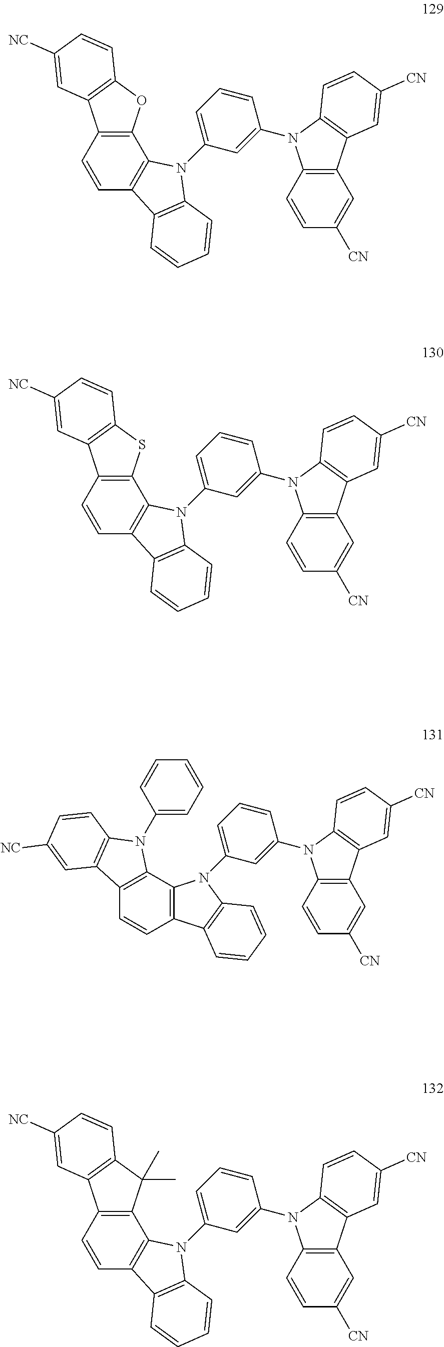 [SMILES: N#Cc1ccc2oc3c(ccc4c5ccccc5n(-c5cccc(-n6c7ccc(C#N)cc7c7cc(C#N)ccc76)c5)c43)c2c1 ; N#Cc1ccc2sc3c(ccc4c5ccccc5n(-c5cccc(-n6c7ccc(C#N)cc7c7cc(C#N)ccc76)c5)c43)c2c1 ; N#Cc1ccc2c(c1)c1cc(C#N)ccc1n2-c1cccc(-n2c3ccccc3c3ccc4c5cc(C#N)ccc5n(-c5ccccc5)c4c32)c1 ; CC1(C)c2ccc(C#N)cc2-c2ccc3c4ccccc4n(-c4cccc(-n5c6ccc(C#N)cc6c6cc(C#N)ccc65)c4)c3c21]

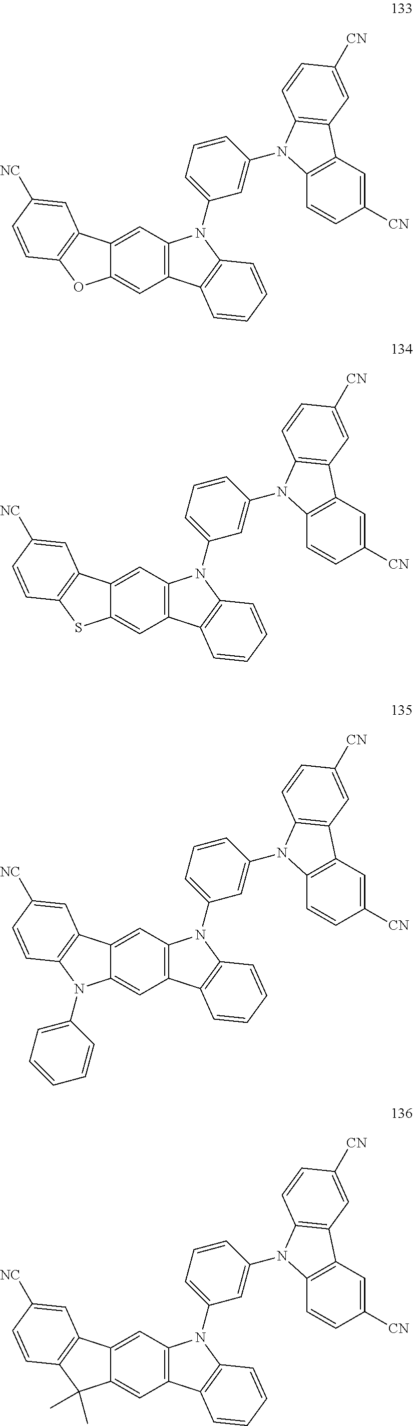 [SMILES: N#Cc1ccc2oc3cc4c5ccccc5n(-c5cccc(-n6c7ccc(C#N)cc7c7cc(C#N)ccc76)c5)c4cc3c2c1 ; N#Cc1ccc2sc3cc4c5ccccc5n(-c5cccc(-n6c7ccc(C#N)cc7c7cc(C#N)ccc76)c5)c4cc3c2c1 ; N#Cc1ccc2c(c1)c1cc(C#N)ccc1n2-c1cccc(-n2c3ccccc3c3cc4c(cc32)c2cc(C#N)ccc2n4-c2ccccc2)c1 ; CC1(C)c2ccc(C#N)cc2-c2cc3c(cc21)c1ccccc1n3-c1cccc(-n2c3ccc(C#N)cc3c3cc(C#N)ccc32)c1]

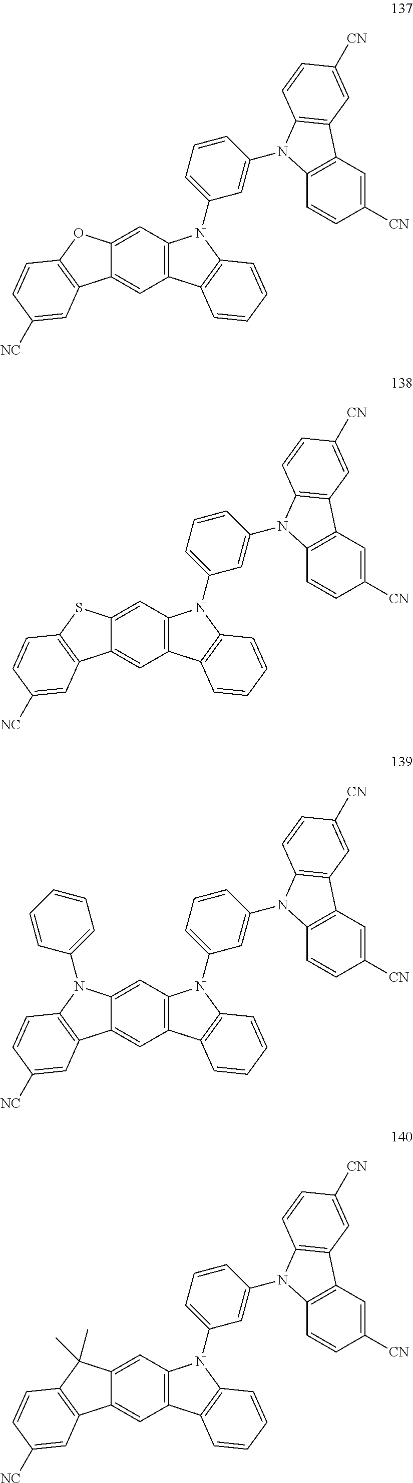 [SMILES: N#Cc1ccc2oc3cc4c(cc3c2c1)c1ccccc1n4-c1cccc(-n2c3ccc(C#N)cc3c3cc(C#N)ccc32)c1 ; N#Cc1ccc2sc3cc4c(cc3c2c1)c1ccccc1n4-c1cccc(-n2c3ccc(C#N)cc3c3cc(C#N)ccc32)c1 ; N#Cc1ccc2c(c1)c1cc(C#N)ccc1n2-c1cccc(-n2c3ccccc3c3cc4c5cc(C#N)ccc5n(-c5ccccc5)c4cc32)c1 ; CC1(C)c2ccc(C#N)cc2-c2cc3c4ccccc4n(-c4cccc(-n5c6ccc(C#N)cc6c6cc(C#N)ccc65)c4)c3cc21]

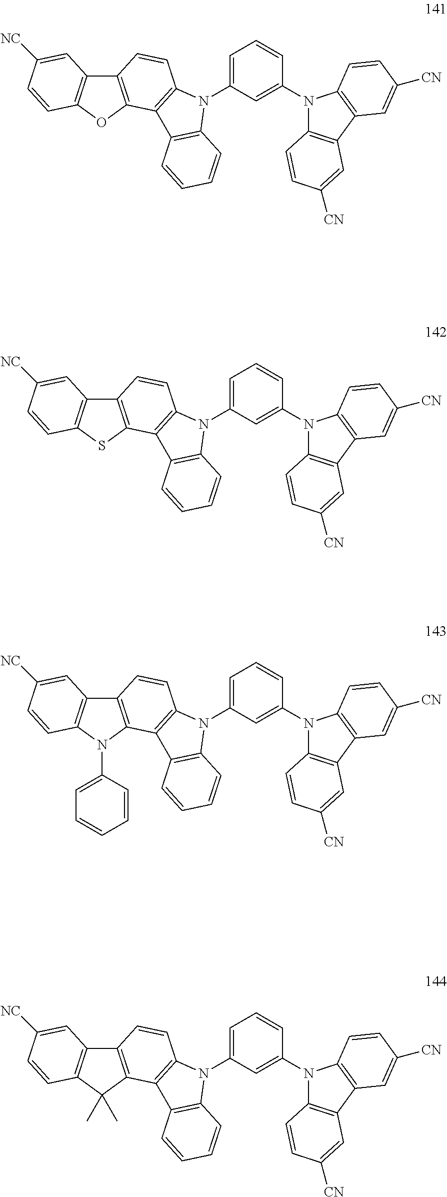 [SMILES: N#Cc1ccc2oc3c(ccc4c3c3ccccc3n4-c3cccc(-n4c5ccc(C#N)cc5c5cc(C#N)ccc54)c3)c2c1 ; N#Cc1ccc2sc3c(ccc4c3c3ccccc3n4-c3cccc(-n4c5ccc(C#N)cc5c5cc(C#N)ccc54)c3)c2c1 ; N#Cc1ccc2c(c1)c1cc(C#N)ccc1n2-c1cccc(-n2c3ccccc3c3c2ccc2c4cc(C#N)ccc4n(-c4ccccc4)c23)c1 ; CC1(C)c2ccc(C#N)cc2-c2ccc3c(c21)c1ccccc1n3-c1cccc(-n2c3ccc(C#N)cc3c3cc(C#N)ccc32)c1]

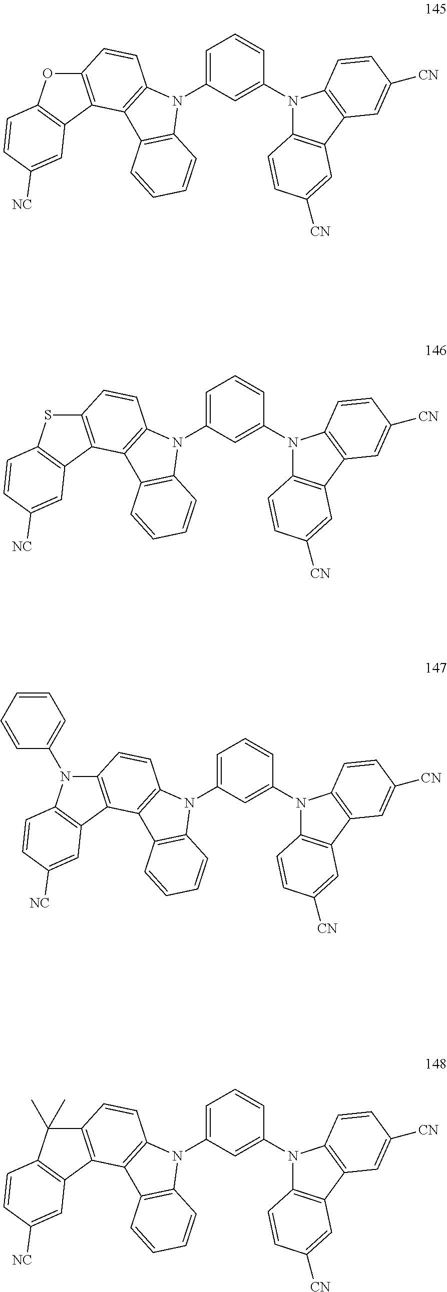 [SMILES: N#Cc1ccc2oc3ccc4c(c5ccccc5n4-c4cccc(-n5c6ccc(C#N)cc6c6cc(C#N)ccc65)c4)c3c2c1 ; N#Cc1ccc2sc3ccc4c(c5ccccc5n4-c4cccc(-n5c6ccc(C#N)cc6c6cc(C#N)ccc65)c4)c3c2c1 ; N#Cc1ccc2c(c1)c1cc(C#N)ccc1n2-c1cccc(-n2c3ccccc3c3c4c5cc(C#N)ccc5n(-c5ccccc5)c4ccc32)c1 ; CC1(C)c2ccc(C#N)cc2-c2c1ccc1c2c2ccccc2n1-c1cccc(-n2c3ccc(C#N)cc3c3cc(C#N)ccc32)c1]

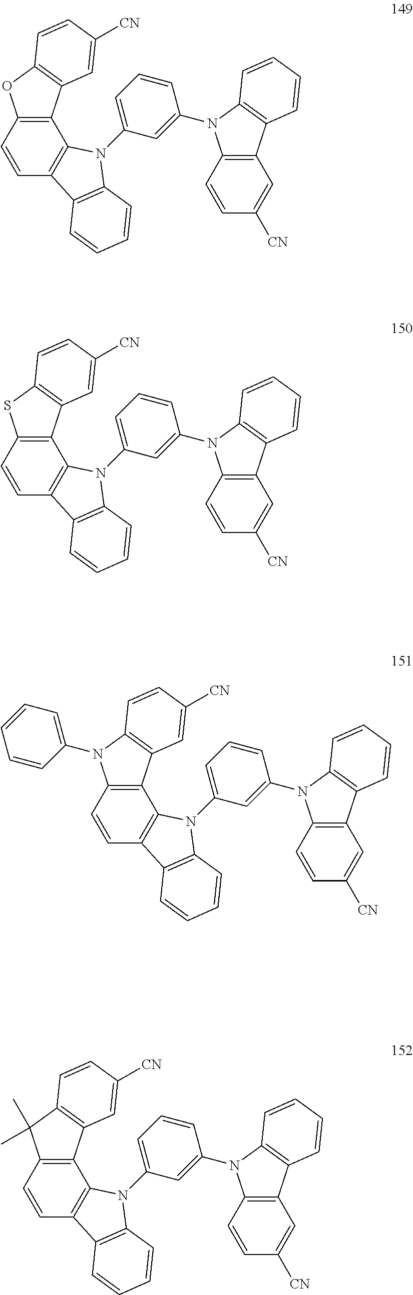 [SMILES: N#Cc1ccc2oc3ccc4c5ccccc5n(-c5cccc(-n6c7ccccc7c7cc(C#N)ccc76)c5)c4c3c2c1 ; N#Cc1ccc2sc3ccc4c5ccccc5n(-c5cccc(-n6c7ccccc7c7cc(C#N)ccc76)c5)c4c3c2c1 ; N#Cc1ccc2c(c1)c1ccccc1n2-c1cccc(-n2c3ccccc3c3ccc4c(c5cc(C#N)ccc5n4-c4ccccc4)c32)c1 ; CC1(C)c2ccc(C#N)cc2-c2c1ccc1c3ccccc3n(-c3cccc(-n4c5ccccc5c5cc(C#N)ccc54)c3)c21]

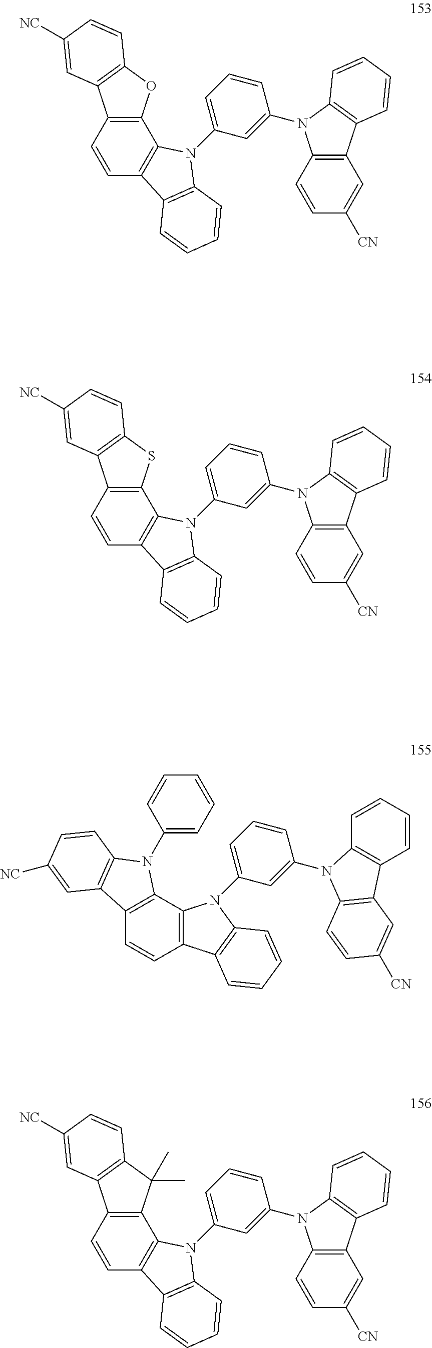 [SMILES: N#Cc1ccc2oc3c(ccc4c5ccccc5n(-c5cccc(-n6c7ccccc7c7cc(C#N)ccc76)c5)c43)c2c1 ; N#Cc1ccc2sc3c(ccc4c5ccccc5n(-c5cccc(-n6c7ccccc7c7cc(C#N)ccc76)c5)c43)c2c1 ; N#Cc1ccc2c(c1)c1ccccc1n2-c1cccc(-n2c3ccccc3c3ccc4c5cc(C#N)ccc5n(-c5ccccc5)c4c32)c1 ; CC1(C)c2ccc(C#N)cc2-c2ccc3c4ccccc4n(-c4cccc(-n5c6ccccc6c6cc(C#N)ccc65)c4)c3c21]

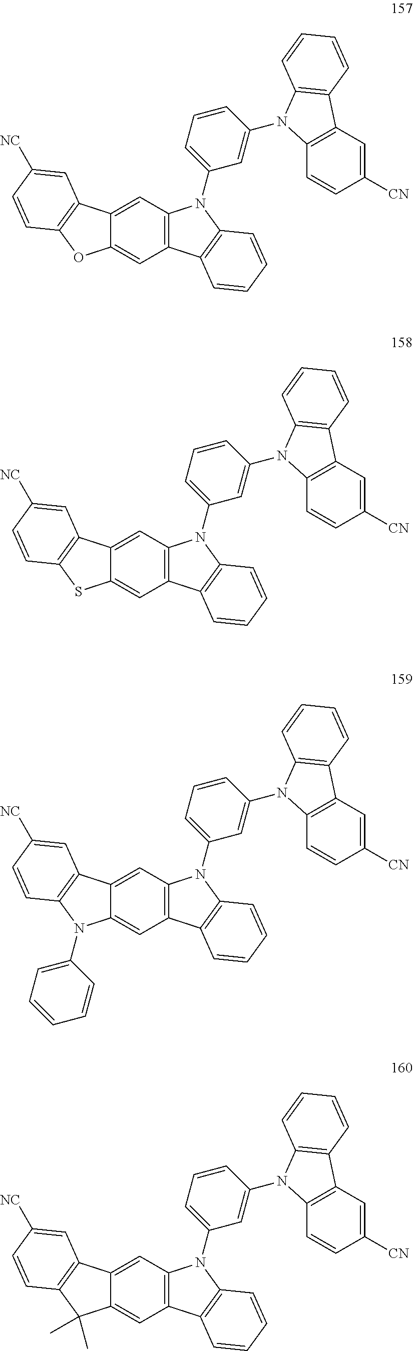 [SMILES: N#Cc1ccc2oc3cc4c5ccccc5n(-c5cccc(-n6c7ccccc7c7cc(C#N)ccc76)c5)c4cc3c2c1 ; N#Cc1ccc2sc3cc4c5ccccc5n(-c5cccc(-n6c7ccccc7c7cc(C#N)ccc76)c5)c4cc3c2c1 ; N#Cc1ccc2c(c1)c1ccccc1n2-c1cccc(-n2c3ccccc3c3cc4c(cc32)c2cc(C#N)ccc2n4-c2ccccc2)c1 ; CC1(C)c2ccc(C#N)cc2-c2cc3c(cc21)c1ccccc1n3-c1cccc(-n2c3ccccc3c3cc(C#N)ccc32)c1]

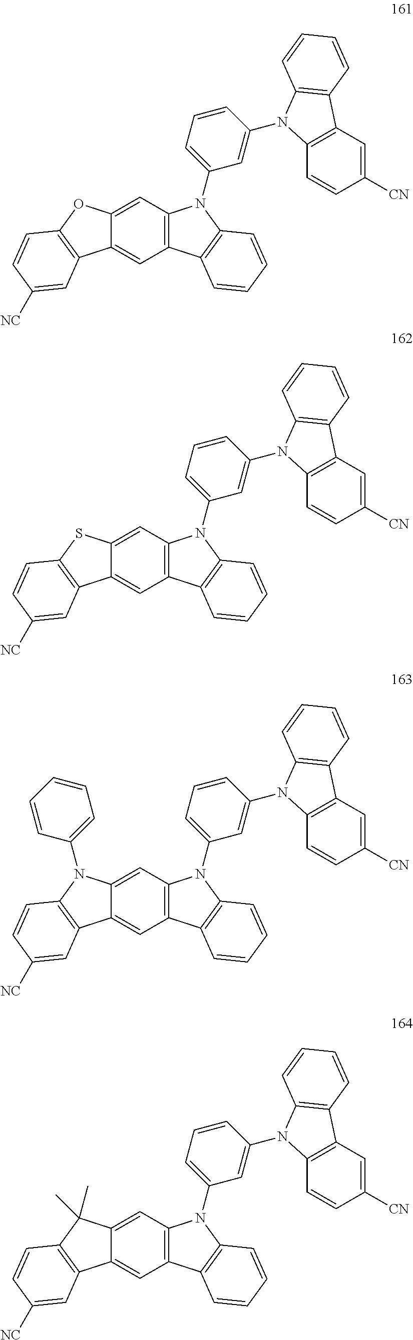 [SMILES: N#Cc1ccc2oc3cc4c(cc3c2c1)c1ccccc1n4-c1cccc(-n2c3ccccc3c3cc(C#N)ccc32)c1 ; N#Cc1ccc2sc3cc4c(cc3c2c1)c1ccccc1n4-c1cccc(-n2c3ccccc3c3cc(C#N)ccc32)c1 ; N#Cc1ccc2c(c1)c1ccccc1n2-c1cccc(-n2c3ccccc3c3cc4c5cc(C#N)ccc5n(-c5ccccc5)c4cc32)c1 ; CC1(C)c2ccc(C#N)cc2-c2cc3c4ccccc4n(-c4cccc(-n5c6ccccc6c6cc(C#N)ccc65)c4)c3cc21]

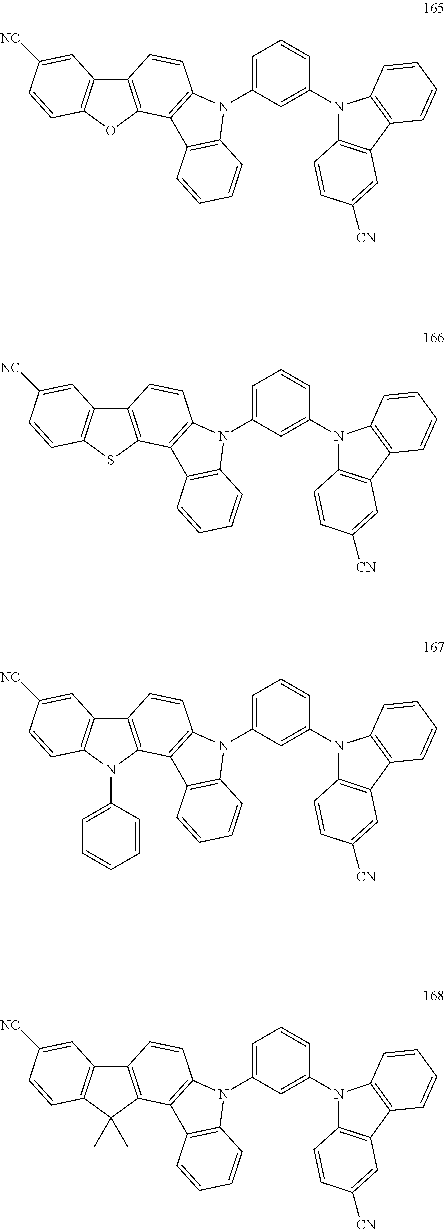 [SMILES: N#Cc1ccc2oc3c(ccc4c3c3ccccc3n4-c3cccc(-n4c5ccccc5c5cc(C#N)ccc54)c3)c2c1 ; N#Cc1ccc2sc3c(ccc4c3c3ccccc3n4-c3cccc(-n4c5ccccc5c5cc(C#N)ccc54)c3)c2c1 ; N#Cc1ccc2c(c1)c1ccccc1n2-c1cccc(-n2c3ccccc3c3c2ccc2c4cc(C#N)ccc4n(-c4ccccc4)c23)c1 ; CC1(C)c2ccc(C#N)cc2-c2ccc3c(c21)c1ccccc1n3-c1cccc(-n2c3ccccc3c3cc(C#N)ccc32)c1]

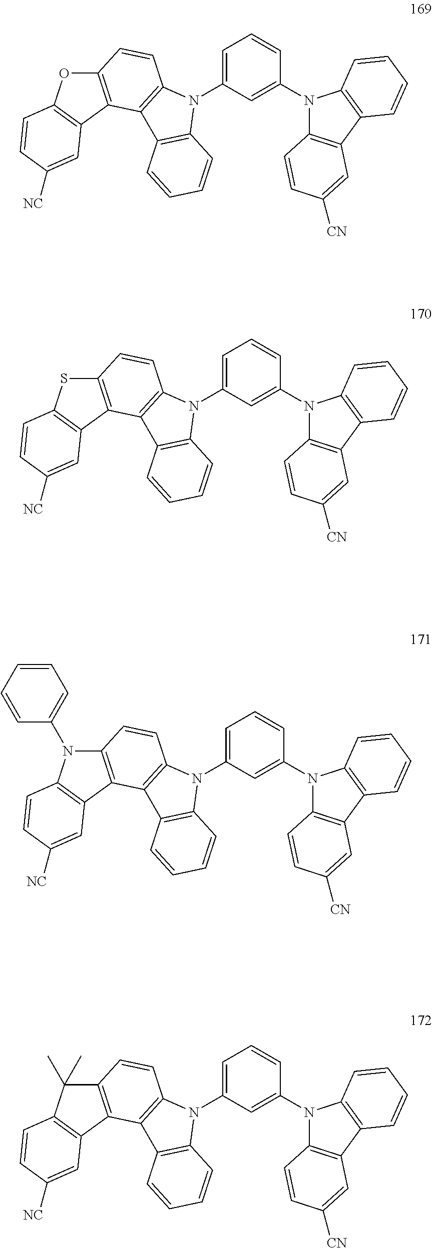 [SMILES: N#Cc1ccc2oc3ccc4c(c5ccccc5n4-c4cccc(-n5c6ccccc6c6cc(C#N)ccc65)c4)c3c2c1 ; N#Cc1ccc2sc3ccc4c(c5ccccc5n4-c4cccc(-n5c6ccccc6c6cc(C#N)ccc65)c4)c3c2c1 ; N#Cc1ccc2c(c1)c1ccccc1n2-c1cccc(-n2c3ccccc3c3c4c5cc(C#N)ccc5n(-c5ccccc5)c4ccc32)c1 ; CC1(C)c2ccc(C#N)cc2-c2c1ccc1c2c2ccccc2n1-c1cccc(-n2c3ccccc3c3cc(C#N)ccc32)c1]

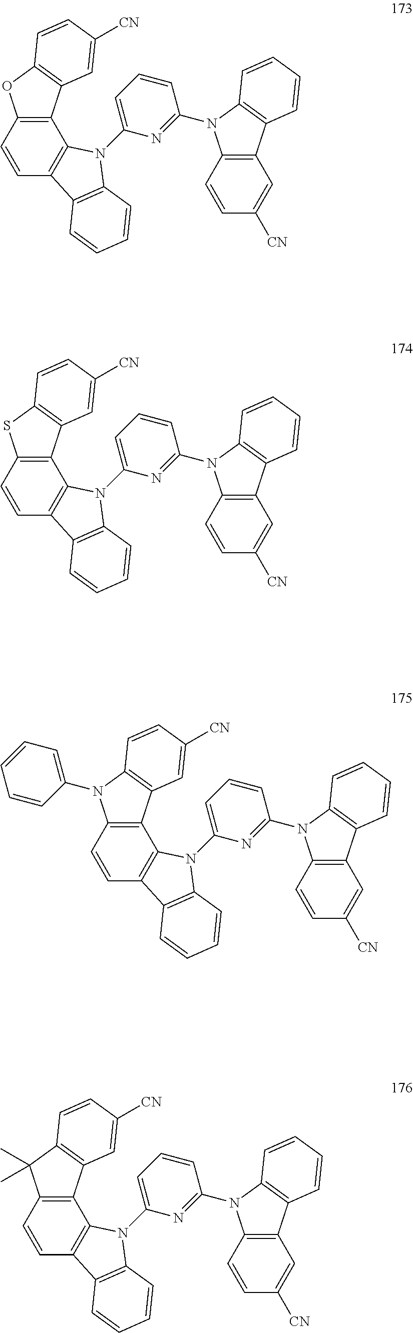 [SMILES: N#Cc1ccc2oc3ccc4c5ccccc5n(-c5cccc(-n6c7ccccc7c7cc(C#N)ccc76)n5)c4c3c2c1 ; N#Cc1ccc2sc3ccc4c5ccccc5n(-c5cccc(-n6c7ccccc7c7cc(C#N)ccc76)n5)c4c3c2c1 ; N#Cc1ccc2c(c1)c1ccccc1n2-c1cccc(-n2c3ccccc3c3ccc4c(c5cc(C#N)ccc5n4-c4ccccc4)c32)n1 ; CC1(C)c2ccc(C#N)cc2-c2c1ccc1c3ccccc3n(-c3cccc(-n4c5ccccc5c5cc(C#N)ccc54)n3)c21]

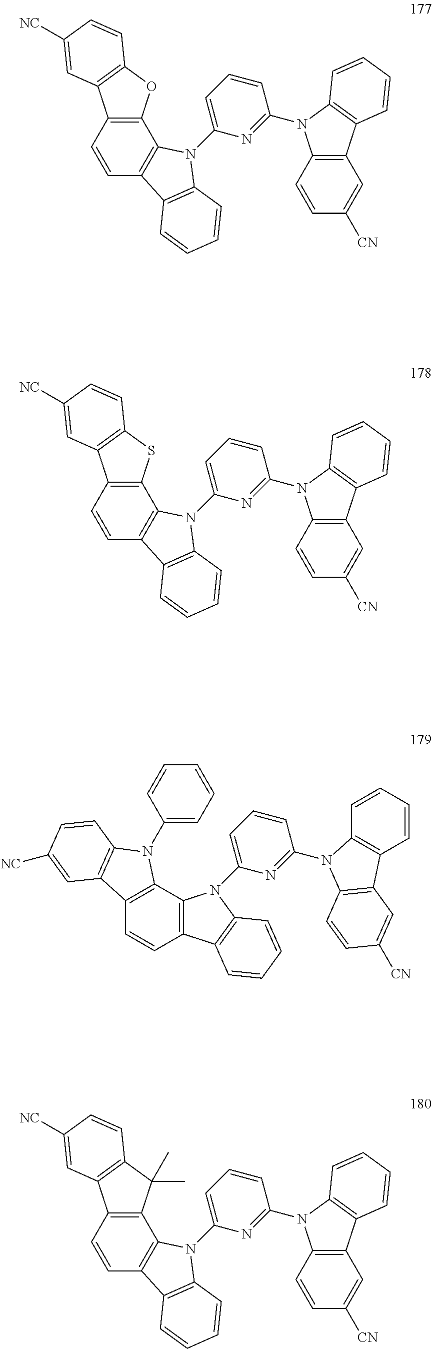 [SMILES: N#Cc1ccc2oc3c(ccc4c5ccccc5n(-c5cccc(-n6c7ccccc7c7cc(C#N)ccc76)n5)c43)c2c1 ; N#Cc1ccc2sc3c(ccc4c5ccccc5n(-c5cccc(-n6c7ccccc7c7cc(C#N)ccc76)n5)c43)c2c1 ; N#Cc1ccc2c(c1)c1ccccc1n2-c1cccc(-n2c3ccccc3c3ccc4c5cc(C#N)ccc5n(-c5ccccc5)c4c32)n1 ; CC1(C)c2ccc(C#N)cc2-c2ccc3c4ccccc4n(-c4cccc(-n5c6ccccc6c6cc(C#N)ccc65)n4)c3c21]

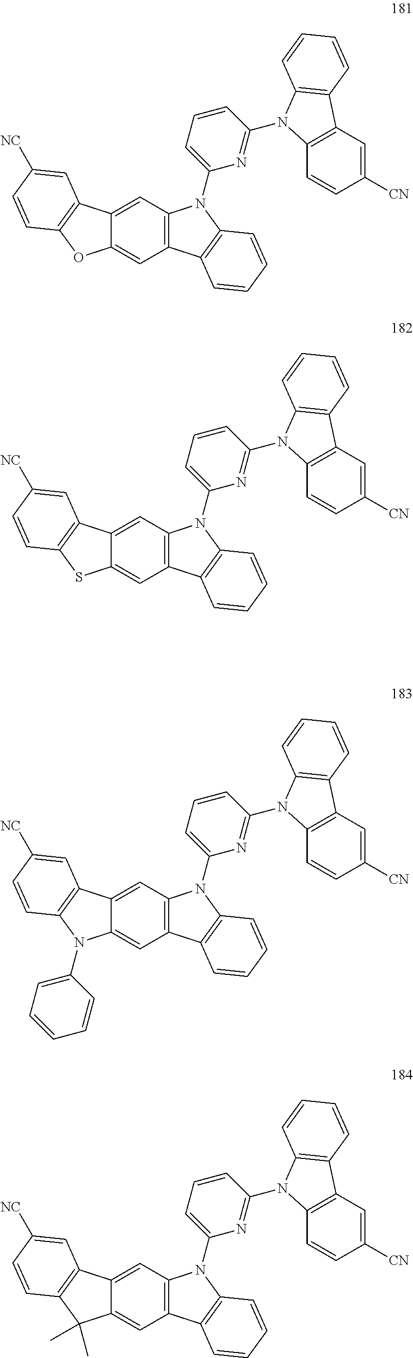 [SMILES: N#Cc1ccc2oc3cc4c5ccccc5n(-c5cccc(-n6c7ccccc7c7cc(C#N)ccc76)n5)c4cc3c2c1 ; N#Cc1ccc2sc3cc4c5ccccc5n(-c5cccc(-n6c7ccccc7c7cc(C#N)ccc76)n5)c4cc3c2c1 ; N#Cc1ccc2c(c1)c1cc3c(cc1n2-c1ccccc1)c1ccccc1n3-c1cccc(-n2c3ccccc3c3cc(C#N)ccc32)n1 ; CC1(C)c2ccc(C#N)cc2-c2cc3c(cc21)c1ccccc1n3-c1cccc(-n2c3ccccc3c3cc(C#N)ccc32)n1]

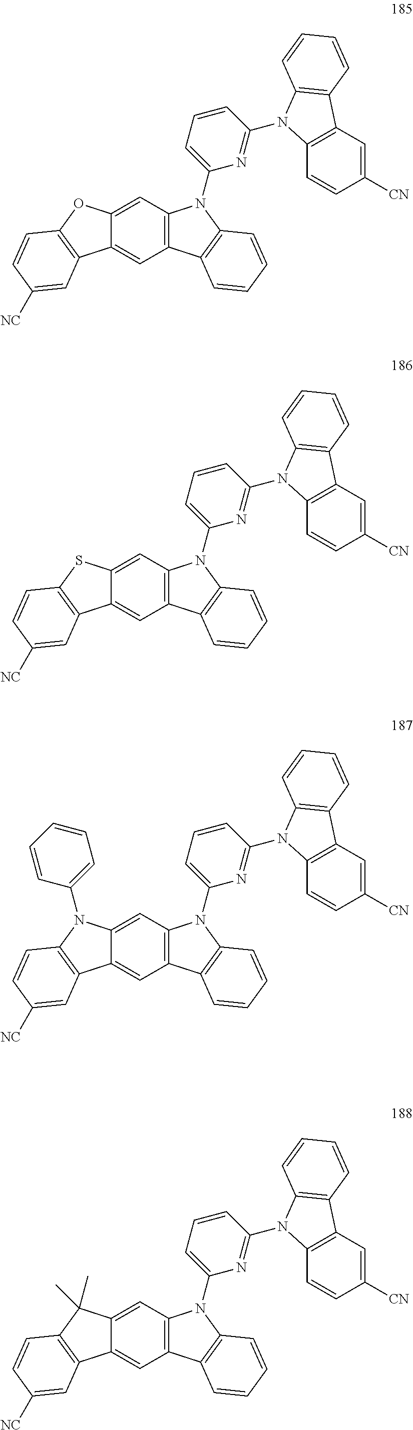 [SMILES: N#Cc1ccc2oc3cc4c(cc3c2c1)c1ccccc1n4-c1cccc(-n2c3ccccc3c3cc(C#N)ccc32)n1 ; N#Cc1ccc2sc3cc4c(cc3c2c1)c1ccccc1n4-c1cccc(-n2c3ccccc3c3cc(C#N)ccc32)n1 ; N#Cc1ccc2c(c1)c1cc3c4ccccc4n(-c4cccc(-n5c6ccccc6c6cc(C#N)ccc65)n4)c3cc1n2-c1ccccc1 ; CC1(C)c2ccc(C#N)cc2-c2cc3c4ccccc4n(-c4cccc(-n5c6ccccc6c6cc(C#N)ccc65)n4)c3cc21]

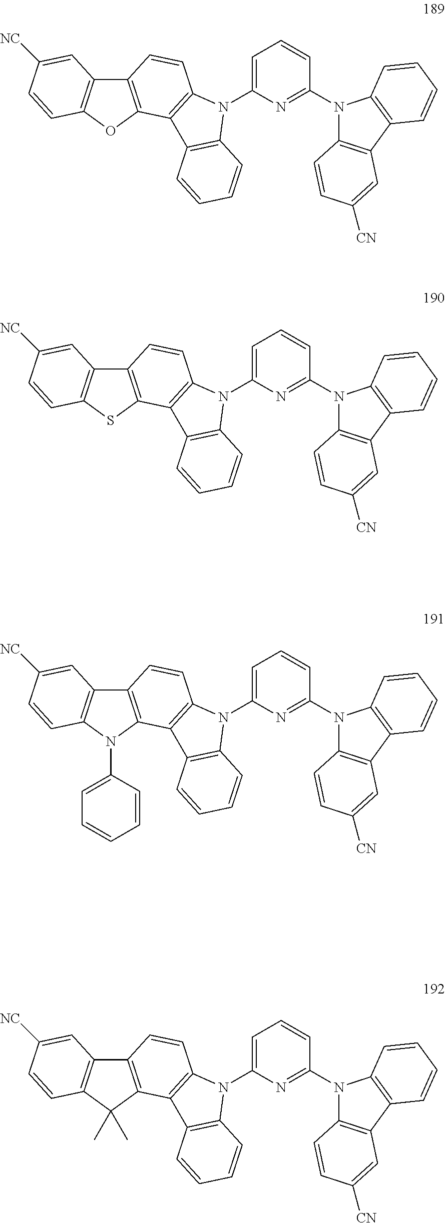 [SMILES: N#Cc1ccc2oc3c(ccc4c3c3ccccc3n4-c3cccc(-n4c5ccccc5c5cc(C#N)ccc54)n3)c2c1 ; N#Cc1ccc2sc3c(ccc4c3c3ccccc3n4-c3cccc(-n4c5ccccc5c5cc(C#N)ccc54)n3)c2c1 ; N#Cc1ccc2c(c1)c1ccccc1n2-c1cccc(-n2c3ccccc3c3c2ccc2c4cc(C#N)ccc4n(-c4ccccc4)c23)n1 ; CC1(C)c2ccc(C#N)cc2-c2ccc3c(c21)c1ccccc1n3-c1cccc(-n2c3ccccc3c3cc(C#N)ccc32)n1]

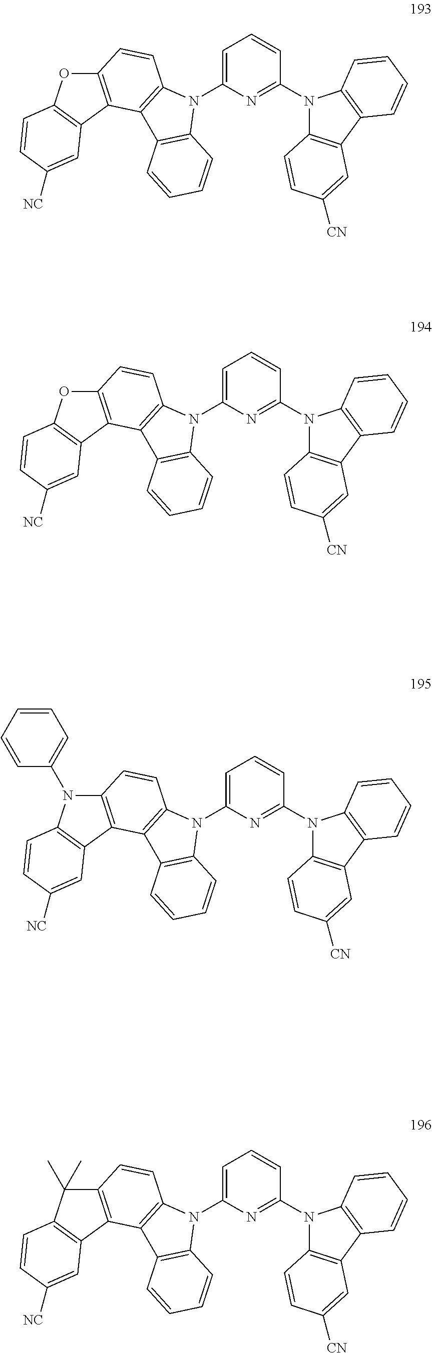 [SMILES: N#Cc1ccc2oc3ccc4c(c5ccccc5n4-c4cccc(-n5c6ccccc6c6cc(C#N)ccc65)n4)c3c2c1 ; N#Cc1ccc2oc3ccc4c(c5ccccc5n4-c4cccc(-n5c6ccccc6c6cc(C#N)ccc65)n4)c3c2c1 ; N#Cc1ccc2c(c1)c1ccccc1n2-c1cccc(-n2c3ccccc3c3c4c5cc(C#N)ccc5n(-c5ccccc5)c4ccc32)n1 ; CC1(C)c2ccc(C#N)cc2-c2c1ccc1c2c2ccccc2n1-c1cccc(-n2c3ccccc3c3cc(C#N)ccc32)n1]

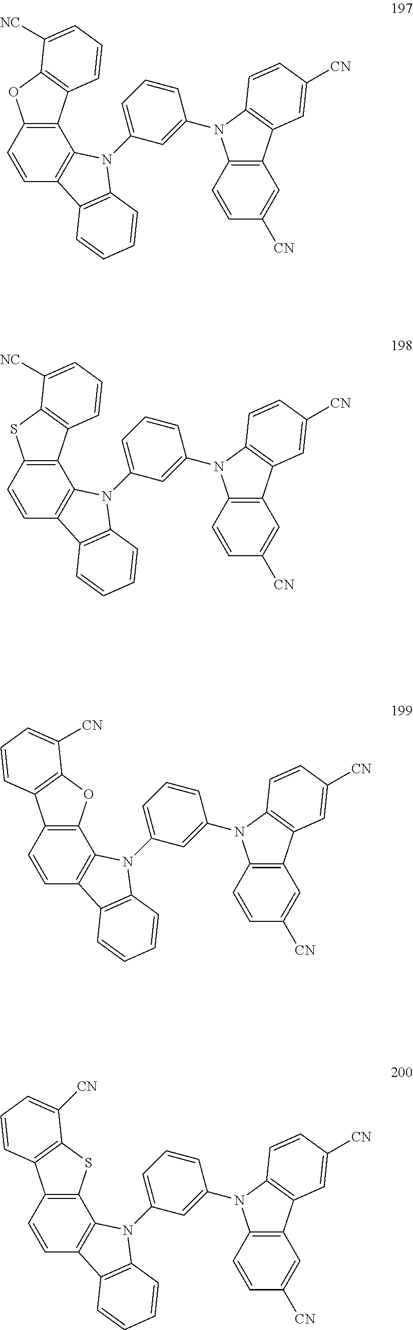 [SMILES: N#Cc1ccc2c(c1)c1cc(C#N)ccc1n2-c1cccc(-n2c3ccccc3c3ccc4oc5c(C#N)cccc5c4c32)c1 ; N#Cc1ccc2c(c1)c1cc(C#N)ccc1n2-c1cccc(-n2c3ccccc3c3ccc4sc5c(C#N)cccc5c4c32)c1 ; N#Cc1ccc2c(c1)c1cc(C#N)ccc1n2-c1cccc(-n2c3ccccc3c3ccc4c5cccc(C#N)c5oc4c32)c1 ; N#Cc1ccc2c(c1)c1cc(C#N)ccc1n2-c1cccc(-n2c3ccccc3c3ccc4c5cccc(C#N)c5sc4c32)c1]

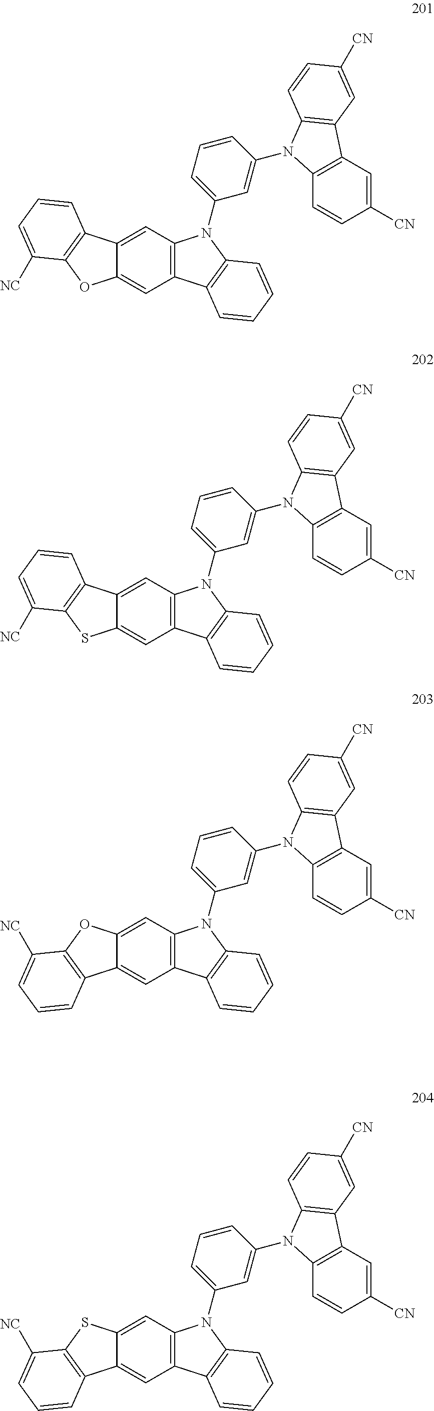 [SMILES: N#Cc1ccc2c(c1)c1cc(C#N)ccc1n2-c1cccc(-n2c3ccccc3c3cc4oc5c(C#N)cccc5c4cc32)c1 ; N#Cc1ccc2c(c1)c1cc(C#N)ccc1n2-c1cccc(-n2c3ccccc3c3cc4sc5c(C#N)cccc5c4cc32)c1 ; N#Cc1ccc2c(c1)c1cc(C#N)ccc1n2-c1cccc(-n2c3ccccc3c3cc4c(cc32)oc2c(C#N)cccc24)c1 ; N#Cc1ccc2c(c1)c1cc(C#N)ccc1n2-c1cccc(-n2c3ccccc3c3cc4c(cc32)sc2c(C#N)cccc24)c1]

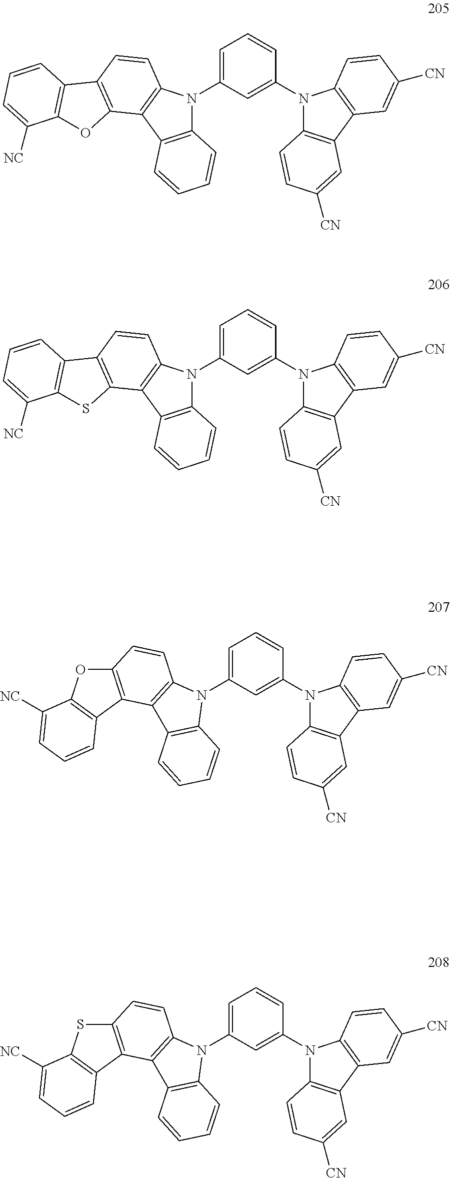 [SMILES: N#Cc1ccc2c(c1)c1cc(C#N)ccc1n2-c1cccc(-n2c3ccccc3c3c4oc5c(C#N)cccc5c4ccc32)c1 ; N#Cc1ccc2c(c1)c1cc(C#N)ccc1n2-c1cccc(-n2c3ccccc3c3c4sc5c(C#N)cccc5c4ccc32)c1 ; N#Cc1ccc2c(c1)c1cc(C#N)ccc1n2-c1cccc(-n2c3ccccc3c3c4c(ccc32)oc2c(C#N)cccc24)c1 ; N#Cc1ccc2c(c1)c1cc(C#N)ccc1n2-c1cccc(-n2c3ccccc3c3c4c(ccc32)sc2c(C#N)cccc24)c1]

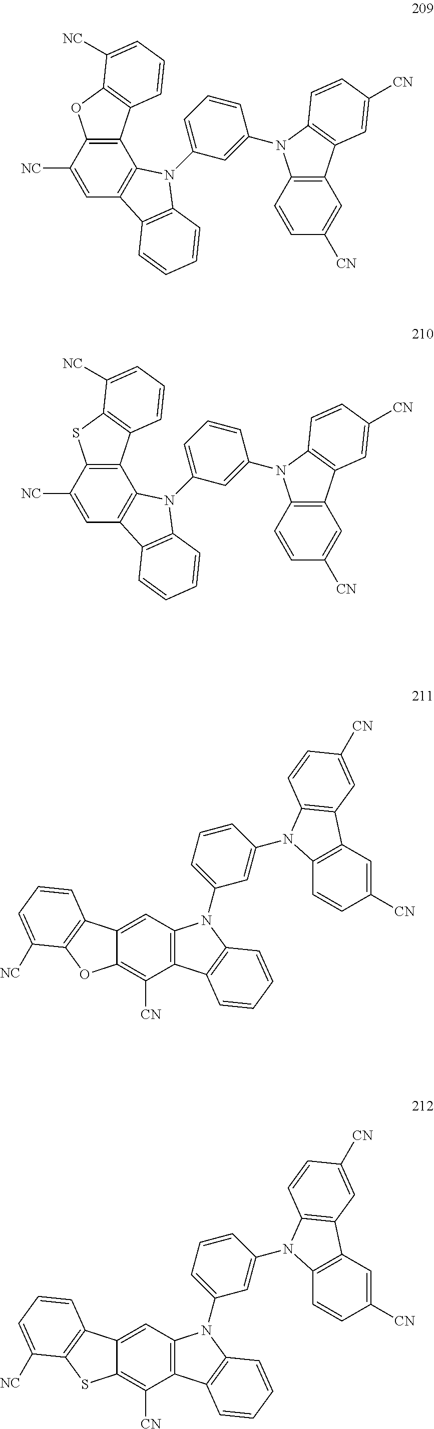 [SMILES: N#Cc1ccc2c(c1)c1cc(C#N)ccc1n2-c1cccc(-n2c3ccccc3c3cc(C#N)c4oc5c(C#N)cccc5c4c32)c1 ; N#Cc1ccc2c(c1)c1cc(C#N)ccc1n2-c1cccc(-n2c3ccccc3c3cc(C#N)c4sc5c(C#N)cccc5c4c32)c1 ; N#Cc1ccc2c(c1)c1cc(C#N)ccc1n2-c1cccc(-n2c3ccccc3c3c(C#N)c4oc5c(C#N)cccc5c4cc32)c1 ; N#Cc1ccc2c(c1)c1cc(C#N)ccc1n2-c1cccc(-n2c3ccccc3c3c(C#N)c4sc5c(C#N)cccc5c4cc32)c1]

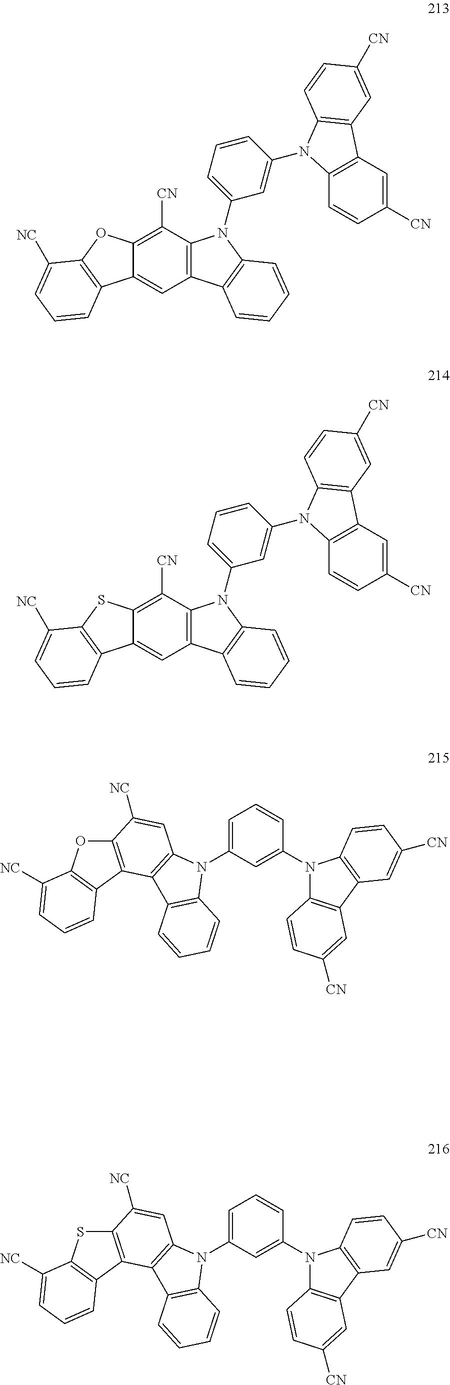 [SMILES: N#Cc1ccc2c(c1)c1cc(C#N)ccc1n2-c1cccc(-n2c3ccccc3c3cc4c(oc5c(C#N)cccc54)c(C#N)c32)c1 ; N#Cc1ccc2c(c1)c1cc(C#N)ccc1n2-c1cccc(-n2c3ccccc3c3cc4c(sc5c(C#N)cccc54)c(C#N)c32)c1 ; N#Cc1ccc2c(c1)c1cc(C#N)ccc1n2-c1cccc(-n2c3ccccc3c3c4c(oc5c(C#N)cccc54)c(C#N)cc32)c1 ; N#Cc1ccc2c(c1)c1cc(C#N)ccc1n2-c1cccc(-n2c3ccccc3c3c4c(sc5c(C#N)cccc54)c(C#N)cc32)c1]

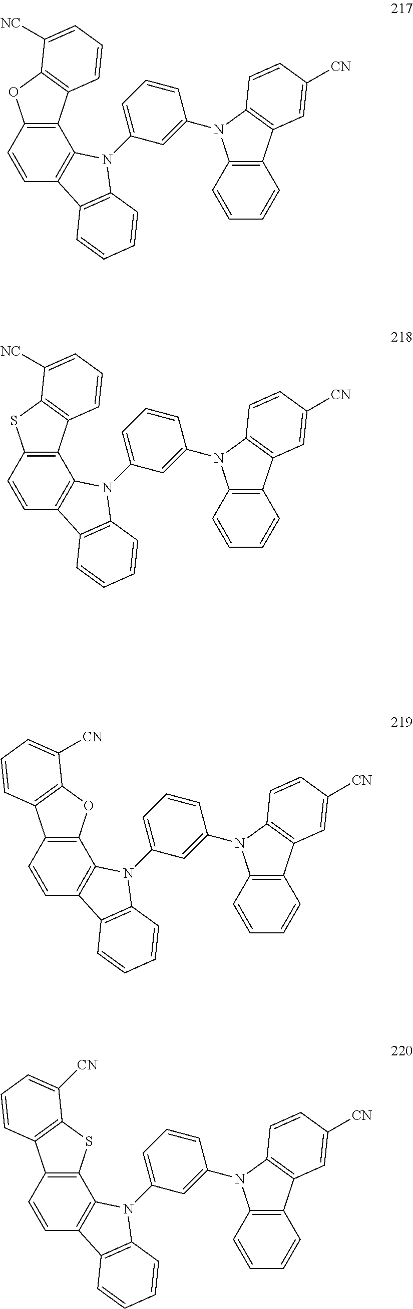 [SMILES: N#Cc1ccc2c(c1)c1ccccc1n2-c1cccc(-n2c3ccccc3c3ccc4oc5c(C#N)cccc5c4c32)c1 ; N#Cc1ccc2c(c1)c1ccccc1n2-c1cccc(-n2c3ccccc3c3ccc4sc5c(C#N)cccc5c4c32)c1 ; N#Cc1ccc2c(c1)c1ccccc1n2-c1cccc(-n2c3ccccc3c3ccc4c5cccc(C#N)c5oc4c32)c1 ; N#Cc1ccc2c(c1)c1ccccc1n2-c1cccc(-n2c3ccccc3c3ccc4c5cccc(C#N)c5sc4c32)c1]

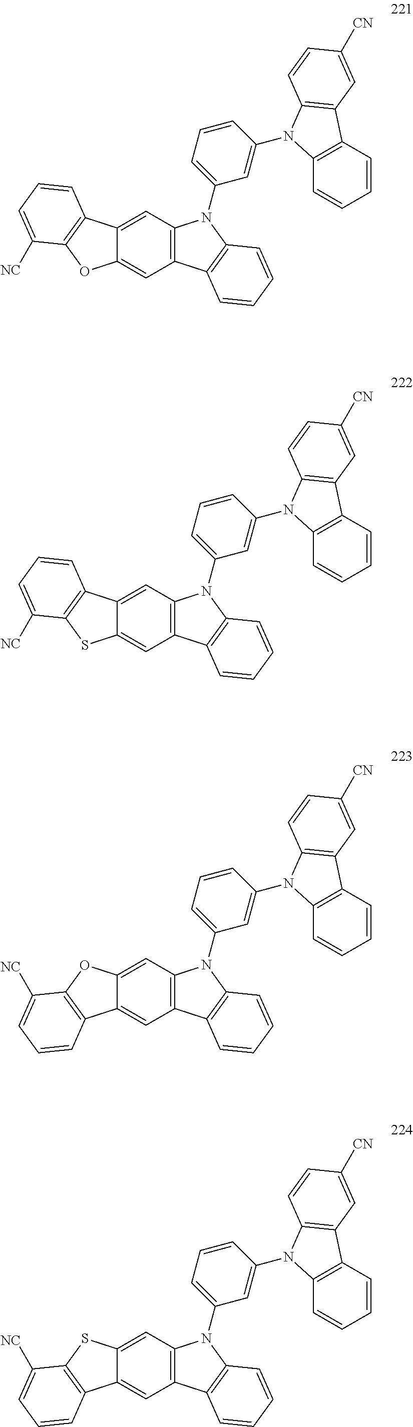 [SMILES: N#Cc1ccc2c(c1)c1ccccc1n2-c1cccc(-n2c3ccccc3c3cc4oc5c(C#N)cccc5c4cc32)c1 ; N#Cc1ccc2c(c1)c1ccccc1n2-c1cccc(-n2c3ccccc3c3cc4sc5c(C#N)cccc5c4cc32)c1 ; N#Cc1ccc2c(c1)c1ccccc1n2-c1cccc(-n2c3ccccc3c3cc4c(cc32)oc2c(C#N)cccc24)c1 ; N#Cc1ccc2c(c1)c1ccccc1n2-c1cccc(-n2c3ccccc3c3cc4c(cc32)sc2c(C#N)cccc24)c1]

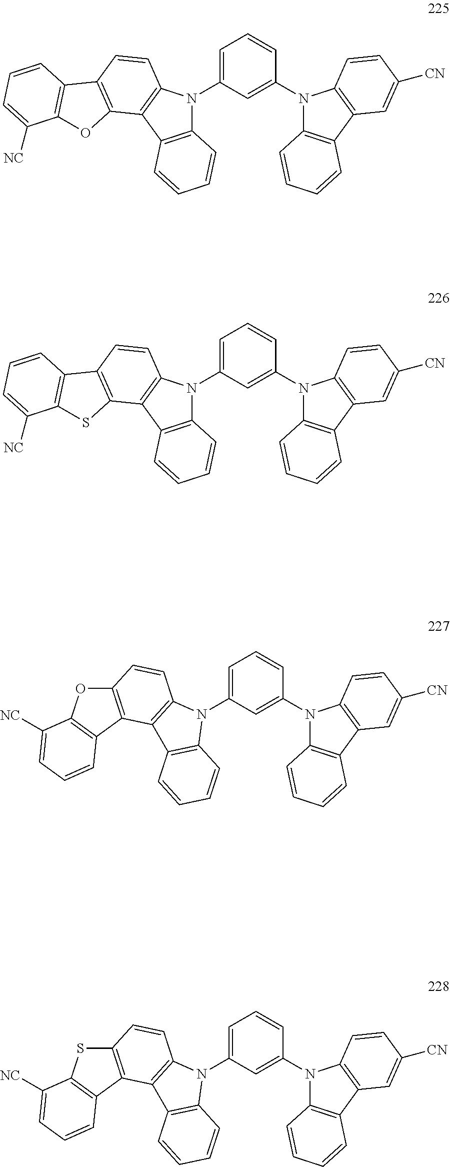 [SMILES: N#Cc1ccc2c(c1)c1ccccc1n2-c1cccc(-n2c3ccccc3c3c4oc5c(C#N)cccc5c4ccc32)c1 ; N#Cc1ccc2c(c1)c1ccccc1n2-c1cccc(-n2c3ccccc3c3c4sc5c(C#N)cccc5c4ccc32)c1 ; N#Cc1ccc2c(c1)c1ccccc1n2-c1cccc(-n2c3ccccc3c3c4c(ccc32)oc2c(C#N)cccc24)c1 ; N#Cc1ccc2c(c1)c1ccccc1n2-c1cccc(-n2c3ccccc3c3c4c(ccc32)sc2c(C#N)cccc24)c1]

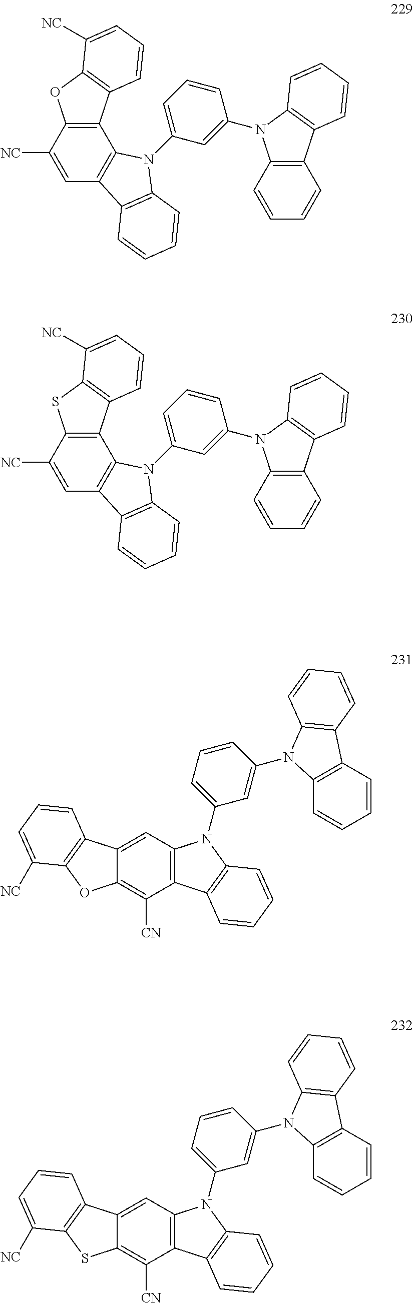 [SMILES: N#Cc1cccc2c1oc1c(C#N)cc3c4ccccc4n(-c4cccc(-n5c6ccccc6c6ccccc65)c4)c3c12 ; N#Cc1cccc2c1sc1c(C#N)cc3c4ccccc4n(-c4cccc(-n5c6ccccc6c6ccccc65)c4)c3c12 ; N#Cc1cccc2c1oc1c(C#N)c3c4ccccc4n(-c4cccc(-n5c6ccccc6c6ccccc65)c4)c3cc12 ; N#Cc1cccc2c1sc1c(C#N)c3c4ccccc4n(-c4cccc(-n5c6ccccc6c6ccccc65)c4)c3cc12]

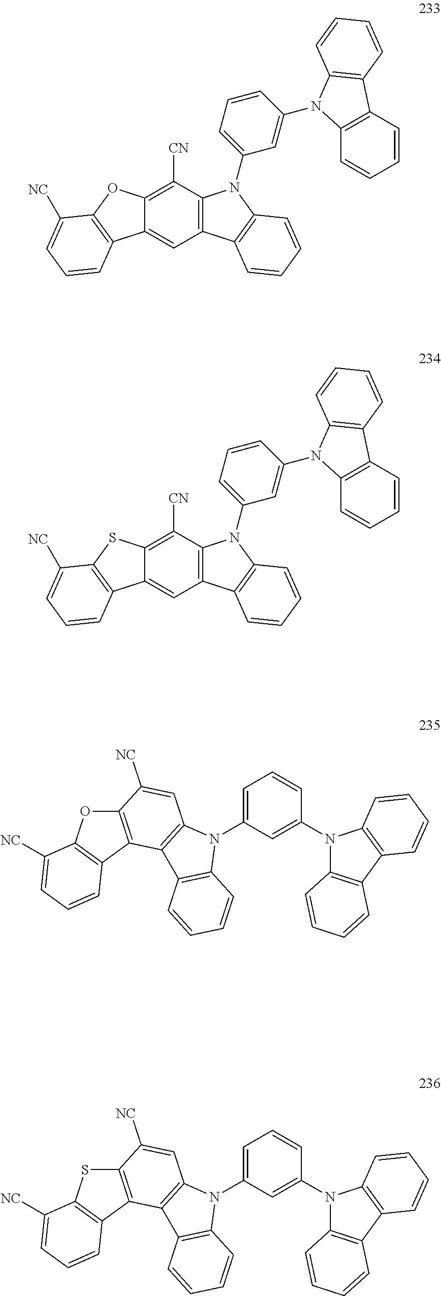 [SMILES: N#Cc1cccc2c1oc1c(C#N)c3c(cc12)c1ccccc1n3-c1cccc(-n2c3ccccc3c3ccccc32)c1 ; N#Cc1cccc2c1sc1c(C#N)c3c(cc12)c1ccccc1n3-c1cccc(-n2c3ccccc3c3ccccc32)c1 ; N#Cc1cccc2c1oc1c(C#N)cc3c(c4ccccc4n3-c3cccc(-n4c5ccccc5c5ccccc54)c3)c12 ; N#Cc1cccc2c1sc1c(C#N)cc3c(c4ccccc4n3-c3cccc(-n4c5ccccc5c5ccccc54)c3)c12]

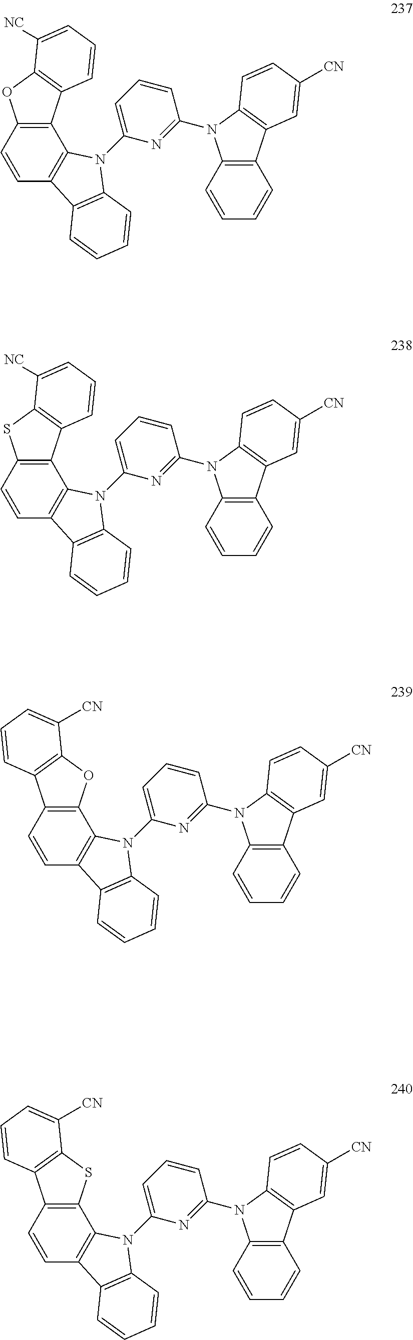

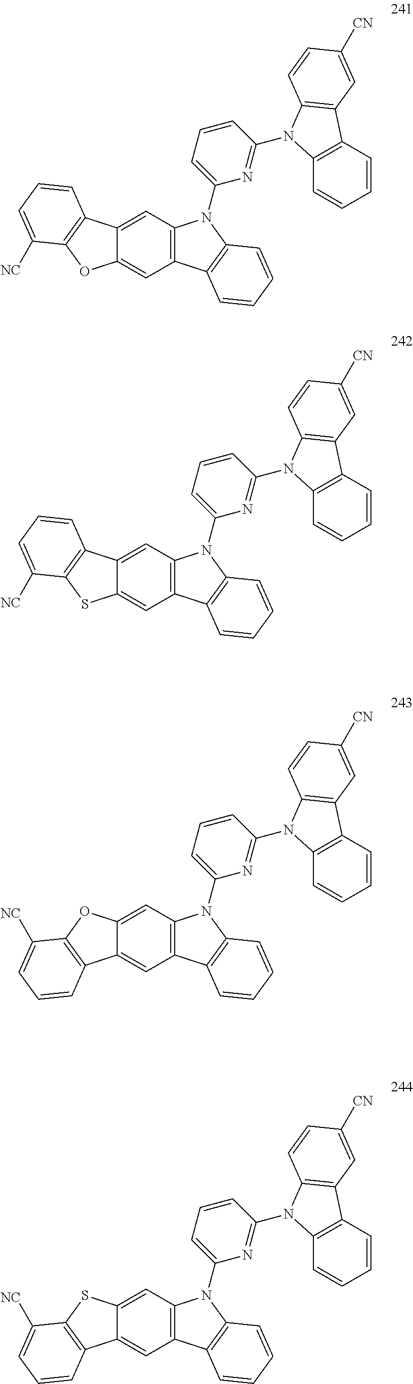 [SMILES: N#Cc1ccc2c(c1)c1ccccc1n2-c1cccc(-n2c3ccccc3c3cc4oc5c(C#N)cccc5c4cc32)n1 ; N#Cc1ccc2c(c1)c1ccccc1n2-c1cccc(-n2c3ccccc3c3cc4sc5c(C#N)cccc5c4cc32)n1 ; N#Cc1ccc2c(c1)c1ccccc1n2-c1cccc(-n2c3ccccc3c3cc4c(cc32)oc2c(C#N)cccc24)n1 ; N#Cc1ccc2c(c1)c1ccccc1n2-c1cccc(-n2c3ccccc3c3cc4c(cc32)sc2c(C#N)cccc24)n1]

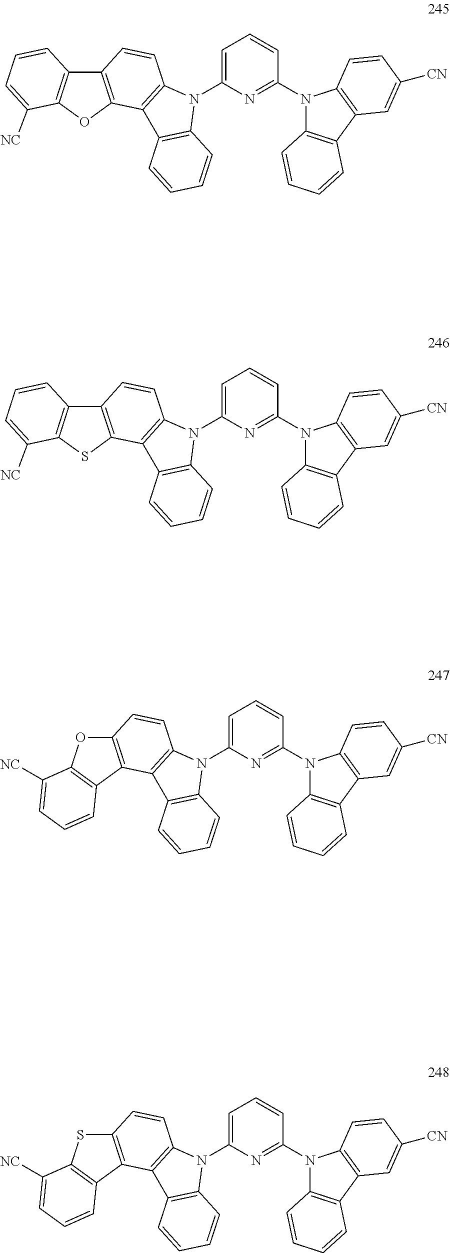 [SMILES: N#Cc1ccc2c(c1)c1ccccc1n2-c1cccc(-n2c3ccccc3c3c4oc5c(C#N)cccc5c4ccc32)n1 ; N#Cc1ccc2c(c1)c1ccccc1n2-c1cccc(-n2c3ccccc3c3c4sc5c(C#N)cccc5c4ccc32)n1 ; N#Cc1ccc2c(c1)c1ccccc1n2-c1cccc(-n2c3ccccc3c3c4c(ccc32)oc2c(C#N)cccc24)n1 ; N#Cc1ccc2c(c1)c1ccccc1n2-c1cccc(-n2c3ccccc3c3c4c(ccc32)sc2c(C#N)cccc24)n1]

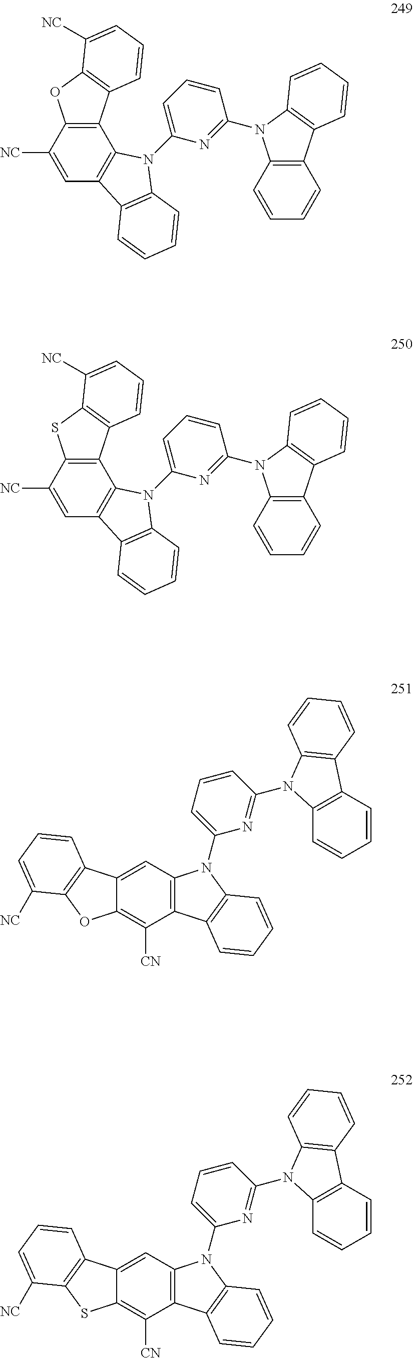 [SMILES: N#Cc1cccc2c1oc1c(C#N)cc3c4ccccc4n(-c4cccc(-n5c6ccccc6c6ccccc65)n4)c3c12 ; N#Cc1cccc2c1sc1c(C#N)cc3c4ccccc4n(-c4cccc(-n5c6ccccc6c6ccccc65)n4)c3c12 ; N#Cc1cccc2c1oc1c(C#N)c3c4ccccc4n(-c4cccc(-n5c6ccccc6c6ccccc65)n4)c3cc12 ; N#Cc1cccc2c1sc1c(C#N)c3c4ccccc4n(-c4cccc(-n5c6ccccc6c6ccccc65)n4)c3cc12]

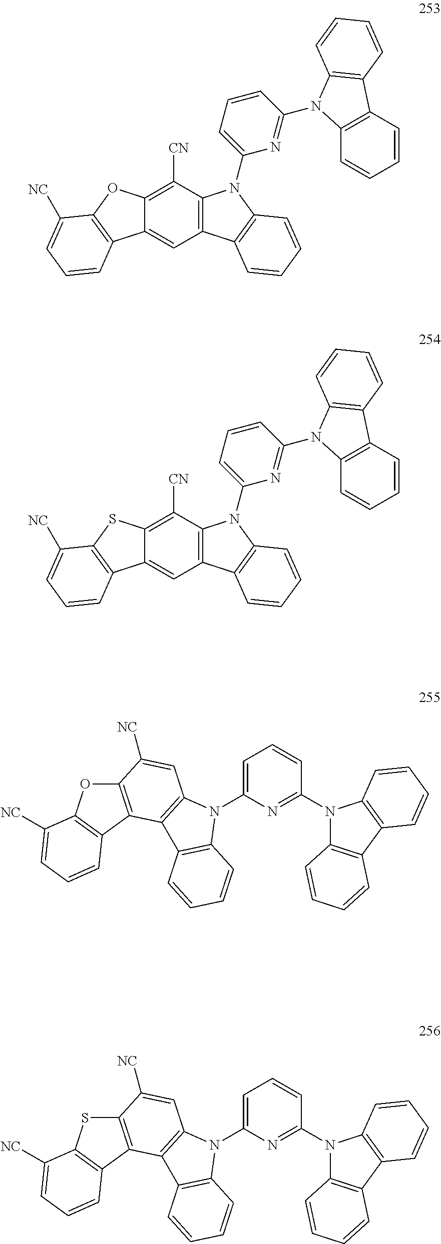 [SMILES: N#Cc1cccc2c1oc1c(C#N)c3c(cc12)c1ccccc1n3-c1cccc(-n2c3ccccc3c3ccccc32)n1 ; N#Cc1cccc2c1sc1c(C#N)c3c(cc12)c1ccccc1n3-c1cccc(-n2c3ccccc3c3ccccc32)n1 ; N#Cc1cccc2c1oc1c(C#N)cc3c(c4ccccc4n3-c3cccc(-n4c5ccccc5c5ccccc54)n3)c12 ; N#Cc1cccc2c1sc1c(C#N)cc3c(c4ccccc4n3-c3cccc(-n4c5ccccc5c5ccccc54)n3)c12]

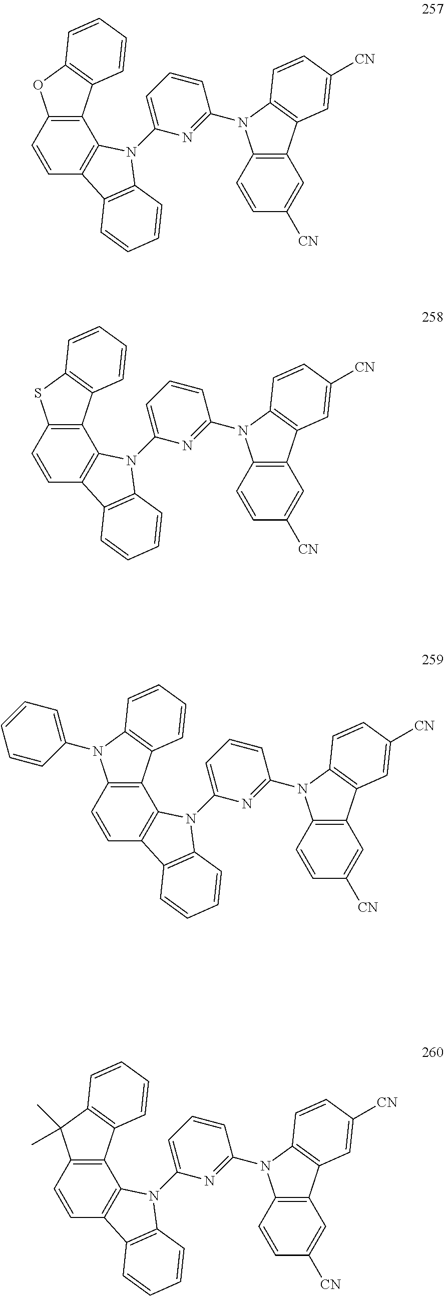 [SMILES: N#Cc1ccc2c(c1)c1cc(C#N)ccc1n2-c1cccc(-n2c3ccccc3c3ccc4oc5ccccc5c4c32)n1 ; N#Cc1ccc2c(c1)c1cc(C#N)ccc1n2-c1cccc(-n2c3ccccc3c3ccc4sc5ccccc5c4c32)n1 ; N#Cc1ccc2c(c1)c1cc(C#N)ccc1n2-c1cccc(-n2c3ccccc3c3ccc4c(c5ccccc5n4-c4ccccc4)c32)n1 ; CC1(C)c2ccccc2-c2c1ccc1c3ccccc3n(-c3cccc(-n4c5ccc(C#N)cc5c5cc(C#N)ccc54)n3)c21]

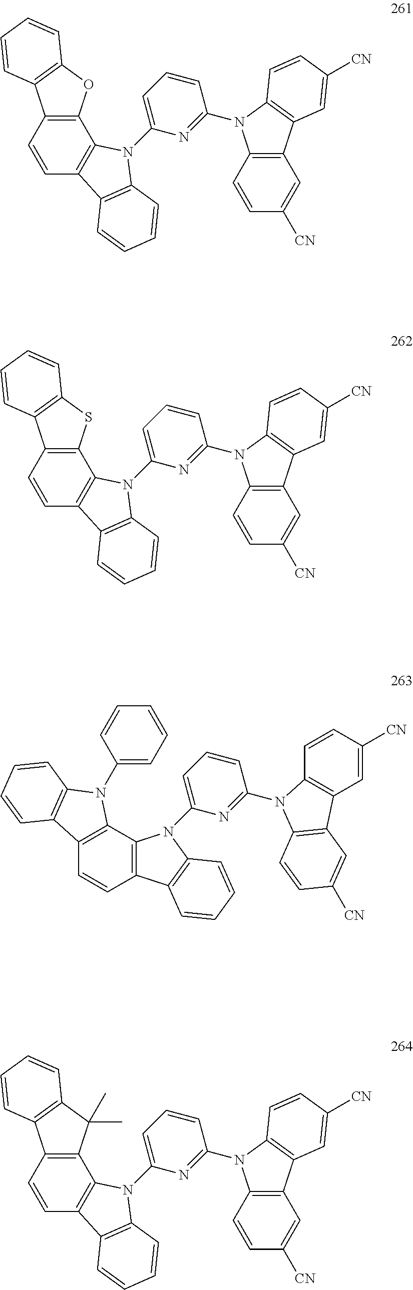 [SMILES: N#Cc1ccc2c(c1)c1cc(C#N)ccc1n2-c1cccc(-n2c3ccccc3c3ccc4c5ccccc5oc4c32)n1 ; N#Cc1ccc2c(c1)c1cc(C#N)ccc1n2-c1cccc(-n2c3ccccc3c3ccc4c5ccccc5sc4c32)n1 ; N#Cc1ccc2c(c1)c1cc(C#N)ccc1n2-c1cccc(-n2c3ccccc3c3ccc4c5ccccc5n(-c5ccccc5)c4c32)n1 ; CC1(C)c2ccccc2-c2ccc3c4ccccc4n(-c4cccc(-n5c6ccc(C#N)cc6c6cc(C#N)ccc65)n4)c3c21]

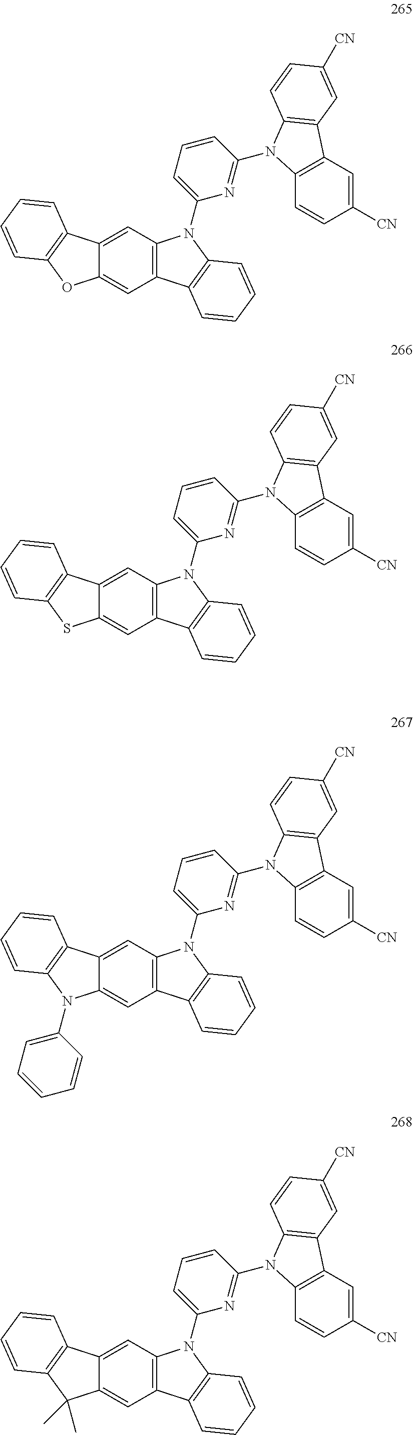 [SMILES: N#Cc1ccc2c(c1)c1cc(C#N)ccc1n2-c1cccc(-n2c3ccccc3c3cc4oc5ccccc5c4cc32)n1 ; N#Cc1ccc2c(c1)c1cc(C#N)ccc1n2-c1cccc(-n2c3ccccc3c3cc4sc5ccccc5c4cc32)n1 ; N#Cc1ccc2c(c1)c1cc(C#N)ccc1n2-c1cccc(-n2c3ccccc3c3cc4c(cc32)c2ccccc2n4-c2ccccc2)n1 ; CC1(C)c2ccccc2-c2cc3c(cc21)c1ccccc1n3-c1cccc(-n2c3ccc(C#N)cc3c3cc(C#N)ccc32)n1]

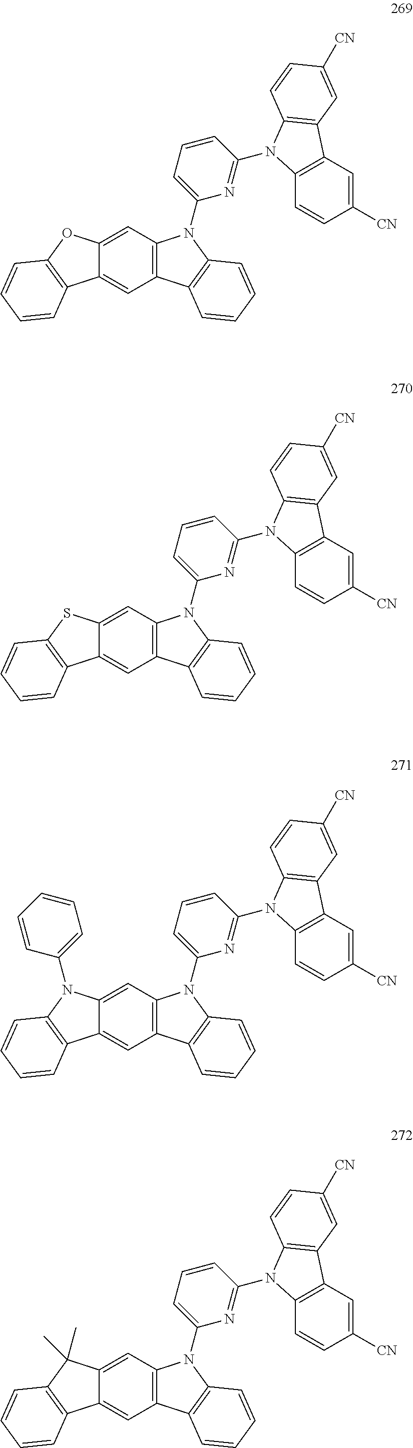 [SMILES: N#Cc1ccc2c(c1)c1cc(C#N)ccc1n2-c1cccc(-n2c3ccccc3c3cc4c(cc32)oc2ccccc24)n1 ; N#Cc1ccc2c(c1)c1cc(C#N)ccc1n2-c1cccc(-n2c3ccccc3c3cc4c(cc32)sc2ccccc24)n1 ; N#Cc1ccc2c(c1)c1cc(C#N)ccc1n2-c1cccc(-n2c3ccccc3c3cc4c5ccccc5n(-c5ccccc5)c4cc32)n1 ; CC1(C)c2ccccc2-c2cc3c4ccccc4n(-c4cccc(-n5c6ccc(C#N)cc6c6cc(C#N)ccc65)n4)c3cc21]

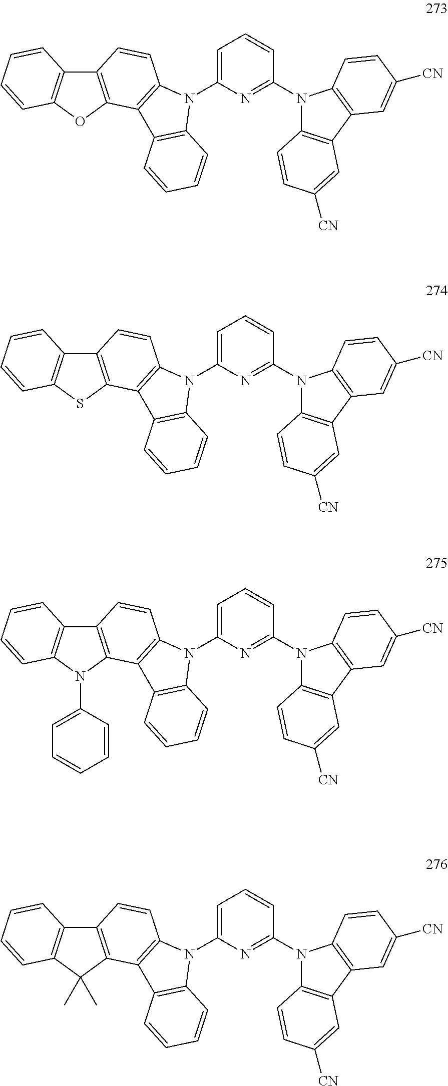 [SMILES: N#Cc1ccc2c(c1)c1cc(C#N)ccc1n2-c1cccc(-n2c3ccccc3c3c4oc5ccccc5c4ccc32)n1 ; N#Cc1ccc2c(c1)c1cc(C#N)ccc1n2-c1cccc(-n2c3ccccc3c3c4sc5ccccc5c4ccc32)n1 ; N#Cc1ccc2c(c1)c1cc(C#N)ccc1n2-c1cccc(-n2c3ccccc3c3c2ccc2c4ccccc4n(-c4ccccc4)c23)n1 ; CC1(C)c2ccccc2-c2ccc3c(c21)c1ccccc1n3-c1cccc(-n2c3ccc(C#N)cc3c3cc(C#N)ccc32)n1]

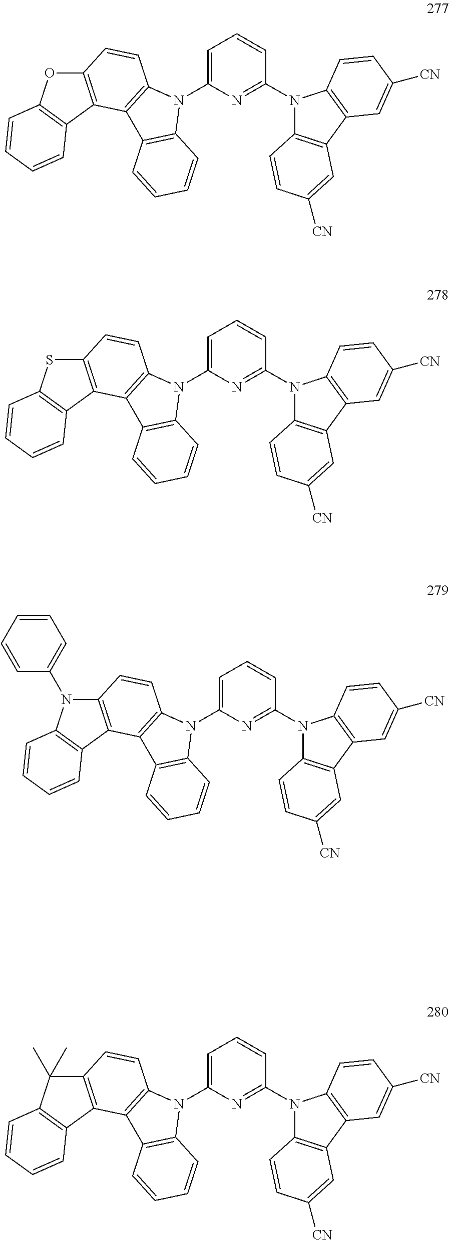 [SMILES: N#Cc1ccc2c(c1)c1cc(C#N)ccc1n2-c1cccc(-n2c3ccccc3c3c4c(ccc32)oc2ccccc24)n1 ; N#Cc1ccc2c(c1)c1cc(C#N)ccc1n2-c1cccc(-n2c3ccccc3c3c4c(ccc32)sc2ccccc24)n1 ; N#Cc1ccc2c(c1)c1cc(C#N)ccc1n2-c1cccc(-n2c3ccccc3c3c4c5ccccc5n(-c5ccccc5)c4ccc32)n1 ; CC1(C)c2ccccc2-c2c1ccc1c2c2ccccc2n1-c1cccc(-n2c3ccc(C#N)cc3c3cc(C#N)ccc32)n1]

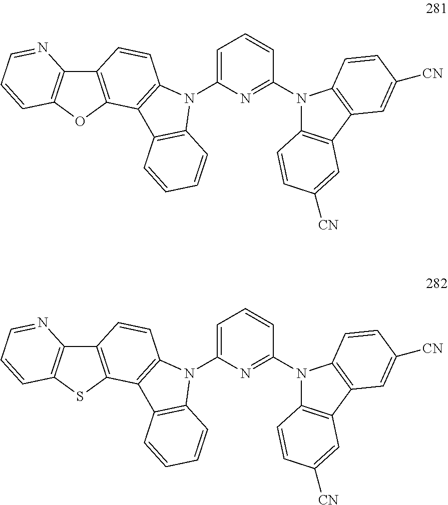 [SMILES: N#Cc1ccc2c(c1)c1cc(C#N)ccc1n2-c1cccc(-n2c3ccccc3c3c4oc5cccnc5c4ccc32)n1 ; N#Cc1ccc2c(c1)c1cc(C#N)ccc1n2-c1cccc(-n2c3ccccc3c3c4sc5cccnc5c4ccc32)n1]

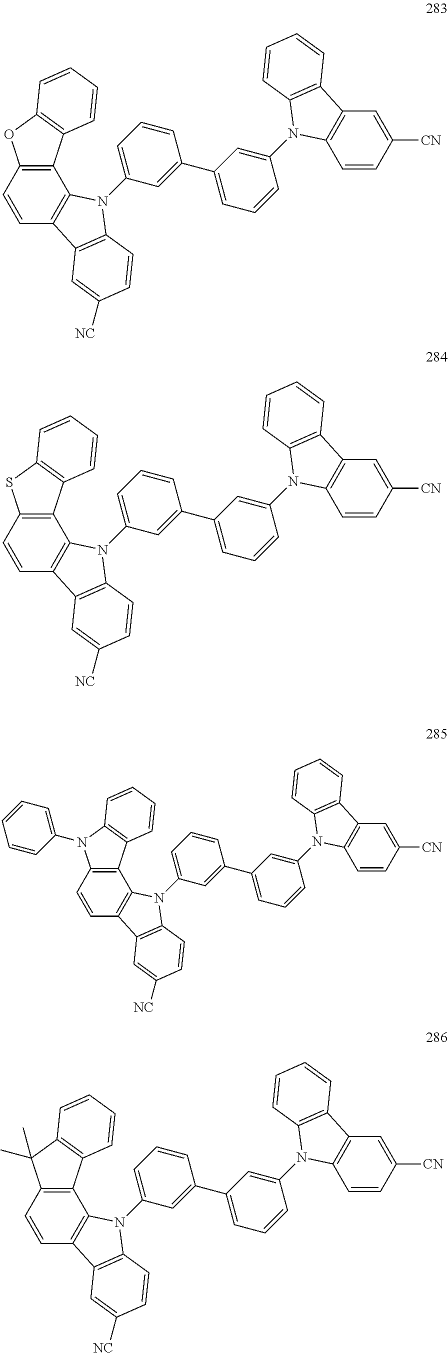 [SMILES: N#Cc1ccc2c(c1)c1ccccc1n2-c1cccc(-c2cccc(-n3c4ccc(C#N)cc4c4ccc5oc6ccccc6c5c43)c2)c1 ; N#Cc1ccc2c(c1)c1ccccc1n2-c1cccc(-c2cccc(-n3c4ccc(C#N)cc4c4ccc5sc6ccccc6c5c43)c2)c1 ; N#Cc1ccc2c(c1)c1ccccc1n2-c1cccc(-c2cccc(-n3c4ccc(C#N)cc4c4ccc5c(c6ccccc6n5-c5ccccc5)c43)c2)c1 ; CC1(C)c2ccccc2-c2c1ccc1c3cc(C#N)ccc3n(-c3cccc(-c4cccc(-n5c6ccccc6c6cc(C#N)ccc65)c4)c3)c21]

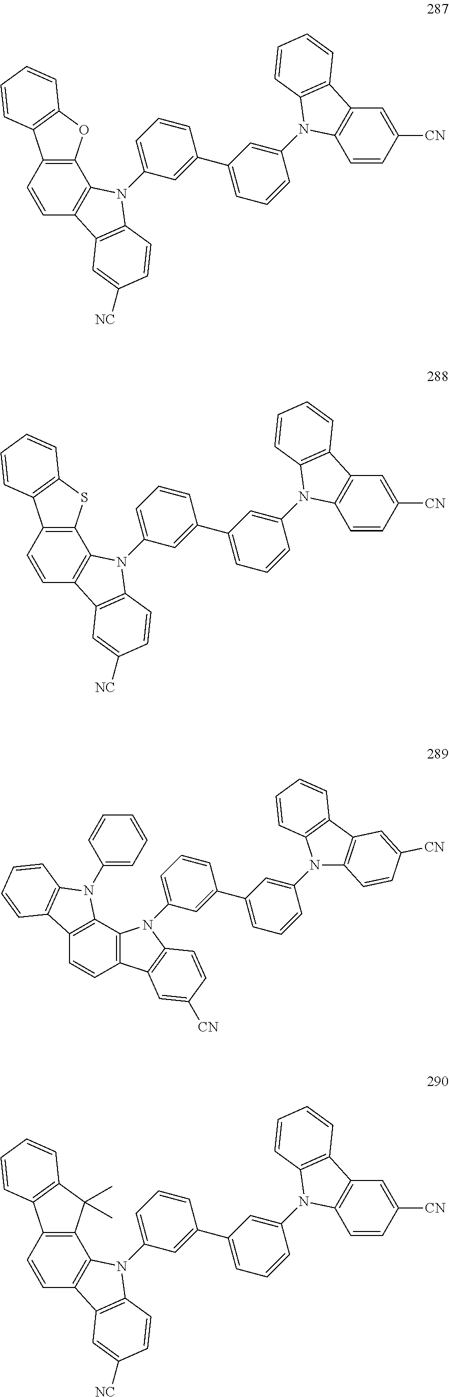 [SMILES: N#Cc1ccc2c(c1)c1ccccc1n2-c1cccc(-c2cccc(-n3c4ccc(C#N)cc4c4ccc5c6ccccc6oc5c43)c2)c1 ; N#Cc1ccc2c(c1)c1ccccc1n2-c1cccc(-c2cccc(-n3c4ccc(C#N)cc4c4ccc5c6ccccc6sc5c43)c2)c1 ; N#Cc1ccc2c(c1)c1ccccc1n2-c1cccc(-c2cccc(-n3c4ccc(C#N)cc4c4ccc5c6ccccc6n(-c6ccccc6)c5c43)c2)c1 ; CC1(C)c2ccccc2-c2ccc3c4cc(C#N)ccc4n(-c4cccc(-c5cccc(-n6c7ccccc7c7cc(C#N)ccc76)c5)c4)c3c21]

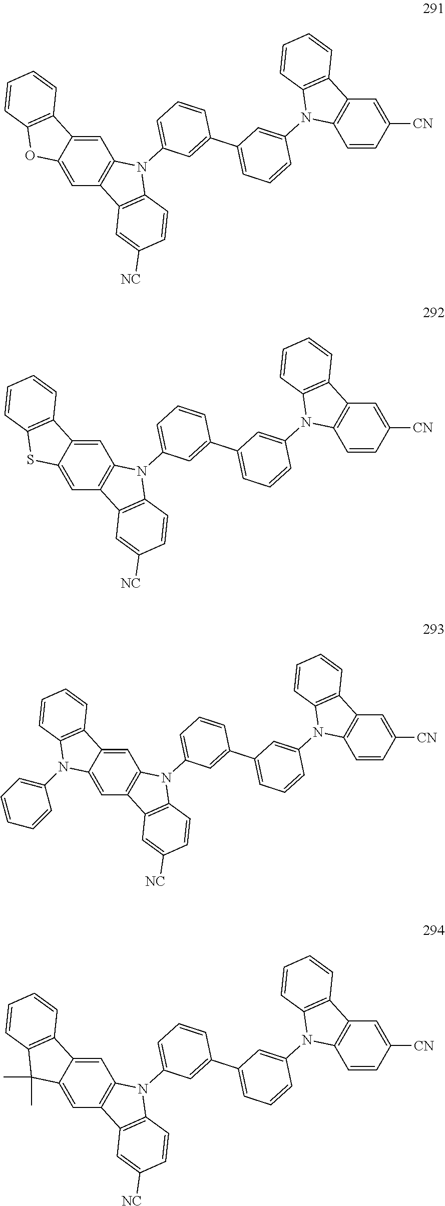 [SMILES: N#Cc1ccc2c(c1)c1ccccc1n2-c1cccc(-c2cccc(-n3c4ccc(C#N)cc4c4cc5oc6ccccc6c5cc43)c2)c1 ; N#Cc1ccc2c(c1)c1ccccc1n2-c1cccc(-c2cccc(-n3c4ccc(C#N)cc4c4cc5sc6ccccc6c5cc43)c2)c1 ; N#Cc1ccc2c(c1)c1ccccc1n2-c1cccc(-c2cccc(-n3c4ccc(C#N)cc4c4cc5c(cc43)c3ccccc3n5-c3ccccc3)c2)c1 ; CC1(C)c2ccccc2-c2cc3c(cc21)c1cc(C#N)ccc1n3-c1cccc(-c2cccc(-n3c4ccccc4c4cc(C#N)ccc43)c2)c1]

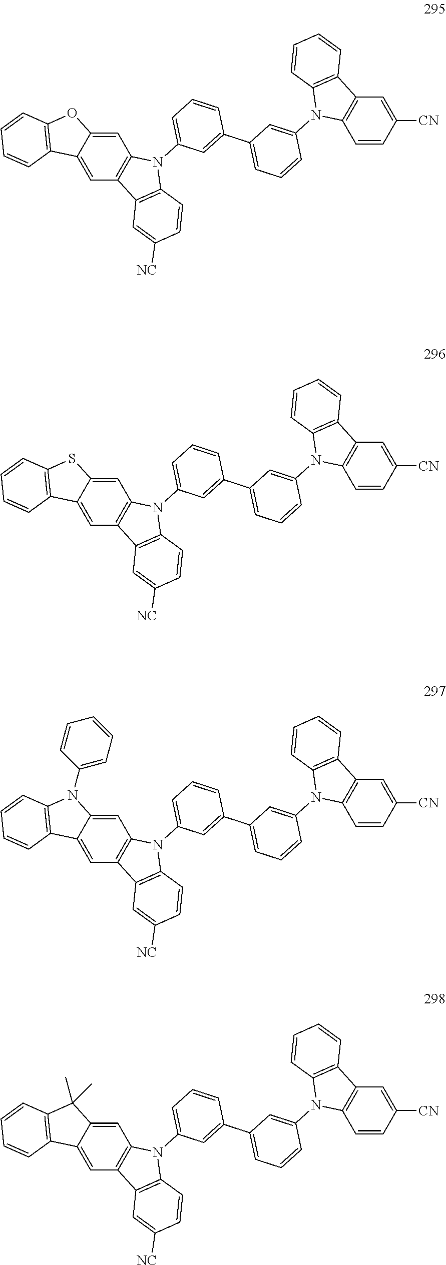 [SMILES: N#Cc1ccc2c(c1)c1ccccc1n2-c1cccc(-c2cccc(-n3c4ccc(C#N)cc4c4cc5c(cc43)oc3ccccc35)c2)c1 ; N#Cc1ccc2c(c1)c1ccccc1n2-c1cccc(-c2cccc(-n3c4ccc(C#N)cc4c4cc5c(cc43)sc3ccccc35)c2)c1 ; N#Cc1ccc2c(c1)c1ccccc1n2-c1cccc(-c2cccc(-n3c4ccc(C#N)cc4c4cc5c6ccccc6n(-c6ccccc6)c5cc43)c2)c1 ; CC1(C)c2ccccc2-c2cc3c4cc(C#N)ccc4n(-c4cccc(-c5cccc(-n6c7ccccc7c7cc(C#N)ccc76)c5)c4)c3cc21]

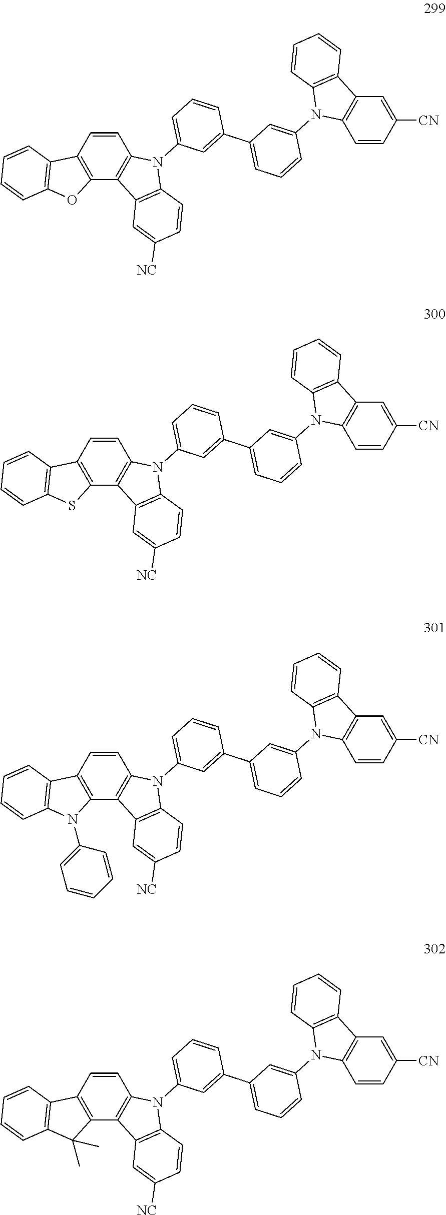 [SMILES: N#Cc1ccc2c(c1)c1ccccc1n2-c1cccc(-c2cccc(-n3c4ccc(C#N)cc4c4c5oc6ccccc6c5ccc43)c2)c1 ; N#Cc1ccc2c(c1)c1ccccc1n2-c1cccc(-c2cccc(-n3c4ccc(C#N)cc4c4c5sc6ccccc6c5ccc43)c2)c1 ; N#Cc1ccc2c(c1)c1ccccc1n2-c1cccc(-c2cccc(-n3c4ccc(C#N)cc4c4c3ccc3c5ccccc5n(-c5ccccc5)c34)c2)c1 ; CC1(C)c2ccccc2-c2ccc3c(c21)c1cc(C#N)ccc1n3-c1cccc(-c2cccc(-n3c4ccccc4c4cc(C#N)ccc43)c2)c1]

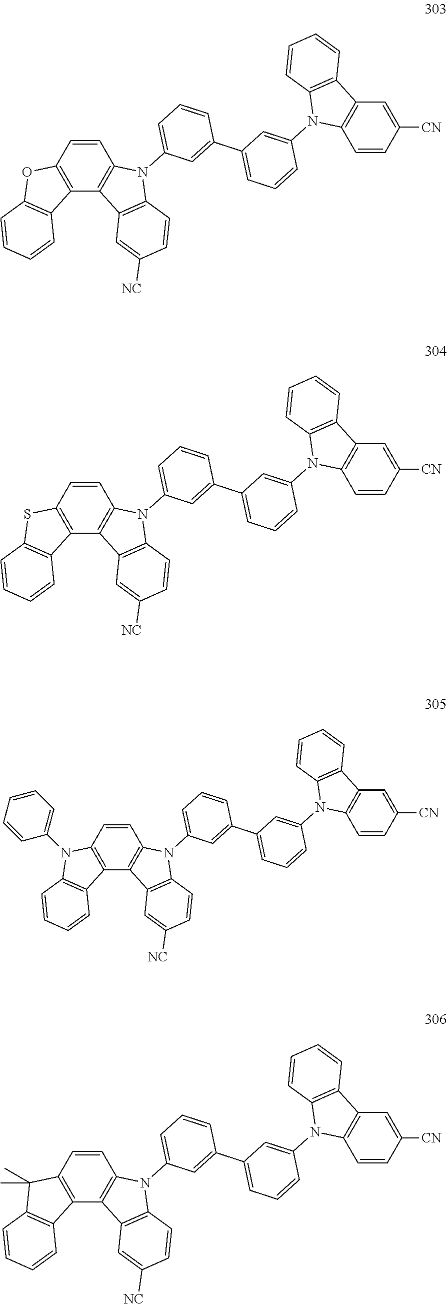 [SMILES: N#Cc1ccc2c(c1)c1ccccc1n2-c1cccc(-c2cccc(-n3c4ccc(C#N)cc4c4c5c(ccc43)oc3ccccc35)c2)c1 ; N#Cc1ccc2c(c1)c1ccccc1n2-c1cccc(-c2cccc(-n3c4ccc(C#N)cc4c4c5c(ccc43)sc3ccccc35)c2)c1 ; N#Cc1ccc2c(c1)c1ccccc1n2-c1cccc(-c2cccc(-n3c4ccc(C#N)cc4c4c5c6ccccc6n(-c6ccccc6)c5ccc43)c2)c1 ; CC1(C)c2ccccc2-c2c1ccc1c2c2cc(C#N)ccc2n1-c1cccc(-c2cccc(-n3c4ccccc4c4cc(C#N)ccc43)c2)c1]

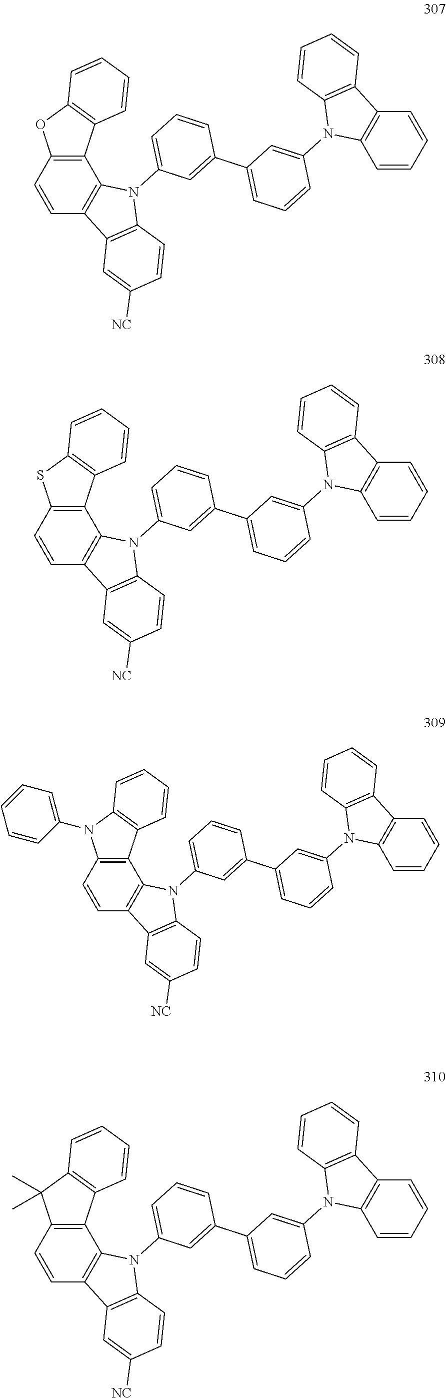 [SMILES: N#Cc1ccc2c(c1)c1ccc3oc4ccccc4c3c1n2-c1cccc(-c2cccc(-n3c4ccccc4c4ccccc43)c2)c1 ; N#Cc1ccc2c(c1)c1ccc3sc4ccccc4c3c1n2-c1cccc(-c2cccc(-n3c4ccccc4c4ccccc43)c2)c1 ; N#Cc1ccc2c(c1)c1ccc3c(c4ccccc4n3-c3ccccc3)c1n2-c1cccc(-c2cccc(-n3c4ccccc4c4ccccc43)c2)c1 ; CC1(C)c2ccccc2-c2c1ccc1c3cc(C#N)ccc3n(-c3cccc(-c4cccc(-n5c6ccccc6c6ccccc65)c4)c3)c21]

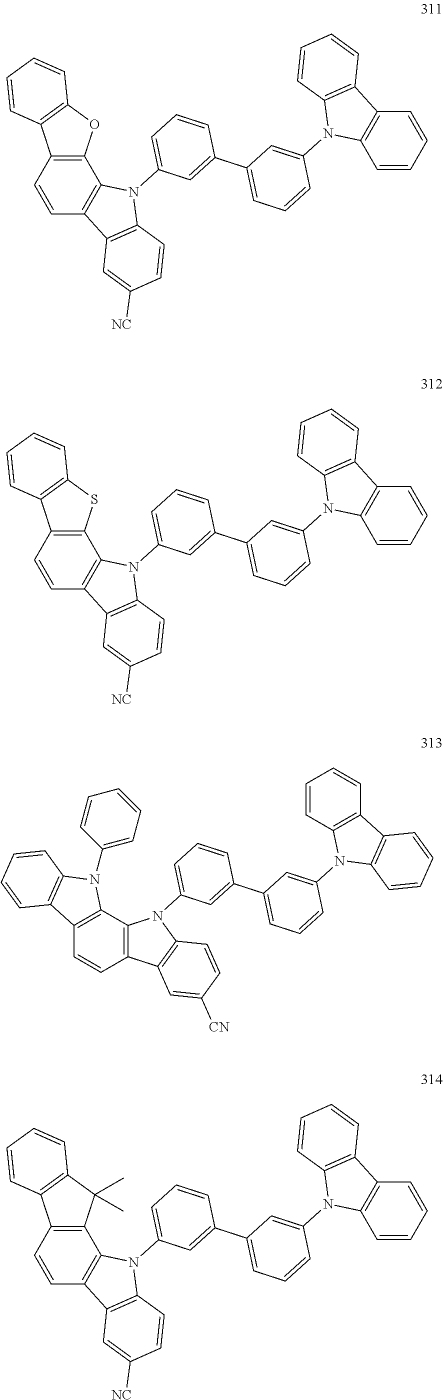 [SMILES: N#Cc1ccc2c(c1)c1ccc3c4ccccc4oc3c1n2-c1cccc(-c2cccc(-n3c4ccccc4c4ccccc43)c2)c1 ; N#Cc1ccc2c(c1)c1ccc3c4ccccc4sc3c1n2-c1cccc(-c2cccc(-n3c4ccccc4c4ccccc43)c2)c1 ; N#Cc1ccc2c(c1)c1ccc3c4ccccc4n(-c4ccccc4)c3c1n2-c1cccc(-c2cccc(-n3c4ccccc4c4ccccc43)c2)c1 ; CC1(C)c2ccccc2-c2ccc3c4cc(C#N)ccc4n(-c4cccc(-c5cccc(-n6c7ccccc7c7ccccc76)c5)c4)c3c21]

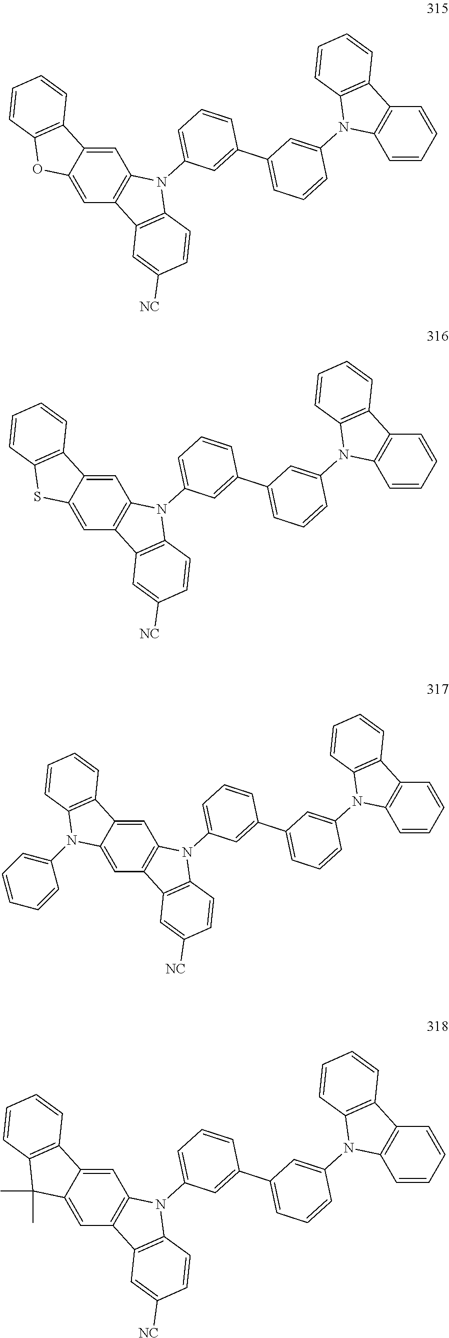 [SMILES: N#Cc1ccc2c(c1)c1cc3oc4ccccc4c3cc1n2-c1cccc(-c2cccc(-n3c4ccccc4c4ccccc43)c2)c1 ; N#Cc1ccc2c(c1)c1cc3sc4ccccc4c3cc1n2-c1cccc(-c2cccc(-n3c4ccccc4c4ccccc43)c2)c1 ; N#Cc1ccc2c(c1)c1cc3c(cc1n2-c1cccc(-c2cccc(-n4c5ccccc5c5ccccc54)c2)c1)c1ccccc1n3-c1ccccc1 ; CC1(C)c2ccccc2-c2cc3c(cc21)c1cc(C#N)ccc1n3-c1cccc(-c2cccc(-n3c4ccccc4c4ccccc43)c2)c1]

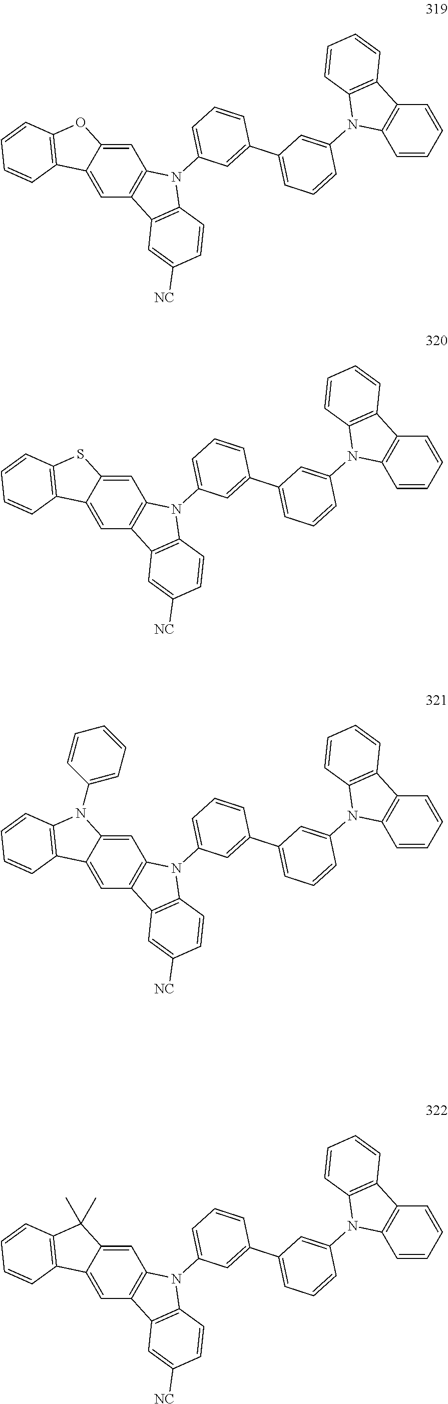 [SMILES: N#Cc1ccc2c(c1)c1cc3c(cc1n2-c1cccc(-c2cccc(-n4c5ccccc5c5ccccc54)c2)c1)oc1ccccc13 ; N#Cc1ccc2c(c1)c1cc3c(cc1n2-c1cccc(-c2cccc(-n4c5ccccc5c5ccccc54)c2)c1)sc1ccccc13 ; N#Cc1ccc2c(c1)c1cc3c4ccccc4n(-c4ccccc4)c3cc1n2-c1cccc(-c2cccc(-n3c4ccccc4c4ccccc43)c2)c1 ; CC1(C)c2ccccc2-c2cc3c4cc(C#N)ccc4n(-c4cccc(-c5cccc(-n6c7ccccc7c7ccccc76)c5)c4)c3cc21]

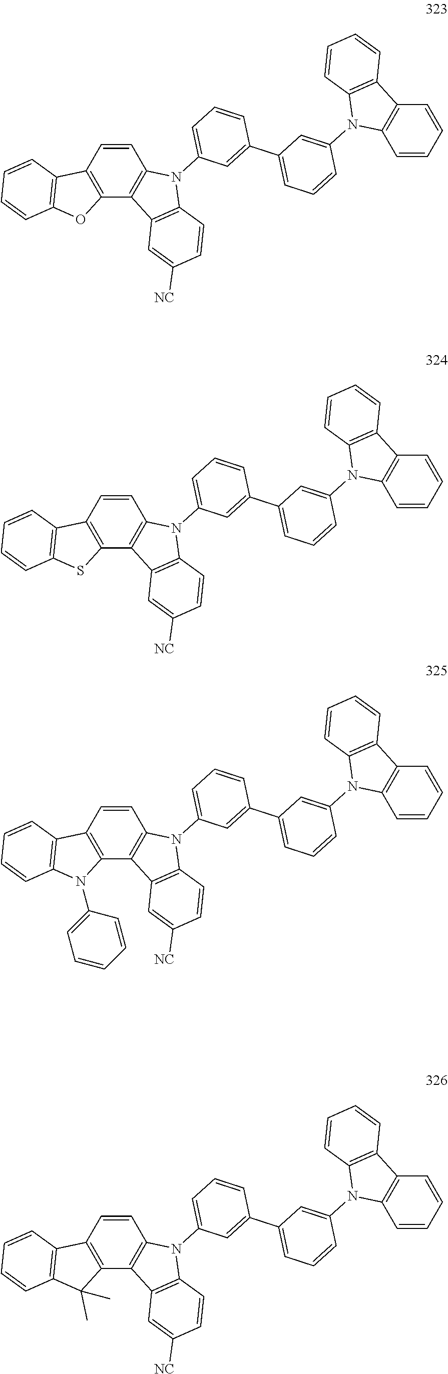 [SMILES: N#Cc1ccc2c(c1)c1c3oc4ccccc4c3ccc1n2-c1cccc(-c2cccc(-n3c4ccccc4c4ccccc43)c2)c1 ; N#Cc1ccc2c(c1)c1c3sc4ccccc4c3ccc1n2-c1cccc(-c2cccc(-n3c4ccccc4c4ccccc43)c2)c1 ; N#Cc1ccc2c(c1)c1c(ccc3c4ccccc4n(-c4ccccc4)c31)n2-c1cccc(-c2cccc(-n3c4ccccc4c4ccccc43)c2)c1 ; CC1(C)c2ccccc2-c2ccc3c(c21)c1cc(C#N)ccc1n3-c1cccc(-c2cccc(-n3c4ccccc4c4ccccc43)c2)c1]

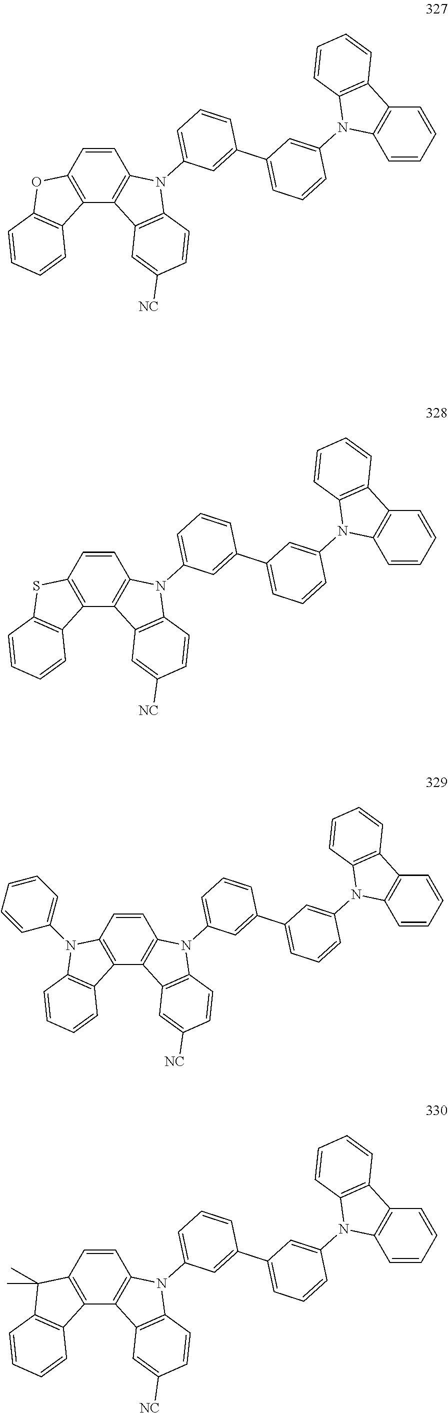 [SMILES: N#Cc1ccc2c(c1)c1c3c(ccc1n2-c1cccc(-c2cccc(-n4c5ccccc5c5ccccc54)c2)c1)oc1ccccc13 ; N#Cc1ccc2c(c1)c1c3c(ccc1n2-c1cccc(-c2cccc(-n4c5ccccc5c5ccccc54)c2)c1)sc1ccccc13 ; N#Cc1ccc2c(c1)c1c3c4ccccc4n(-c4ccccc4)c3ccc1n2-c1cccc(-c2cccc(-n3c4ccccc4c4ccccc43)c2)c1 ; CC1(C)c2ccccc2-c2c1ccc1c2c2cc(C#N)ccc2n1-c1cccc(-c2cccc(-n3c4ccccc4c4ccccc43)c2)c1]

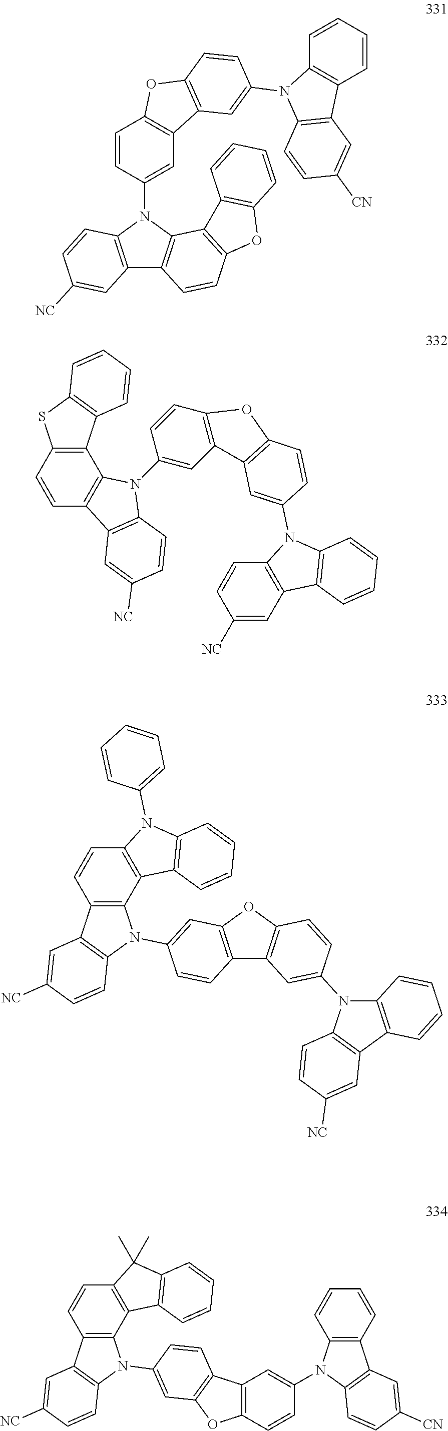 [SMILES: N#Cc1ccc2c(c1)c1ccccc1n2-c1ccc2oc3ccc(-n4c5ccc(C#N)cc5c5ccc6oc7ccccc7c6c54)cc3c2c1 ; N#Cc1ccc2c(c1)c1ccccc1n2-c1ccc2oc3ccc(-n4c5ccc(C#N)cc5c5ccc6sc7ccccc7c6c54)cc3c2c1 ; N#Cc1ccc2c(c1)c1ccccc1n2-c1ccc2oc3cc(-n4c5ccc(C#N)cc5c5ccc6c(c7ccccc7n6-c6ccccc6)c54)ccc3c2c1 ; CC1(C)c2ccccc2-c2c1ccc1c3cc(C#N)ccc3n(-c3ccc4c(c3)oc3ccc(-n5c6ccccc6c6cc(C#N)ccc65)cc34)c21]

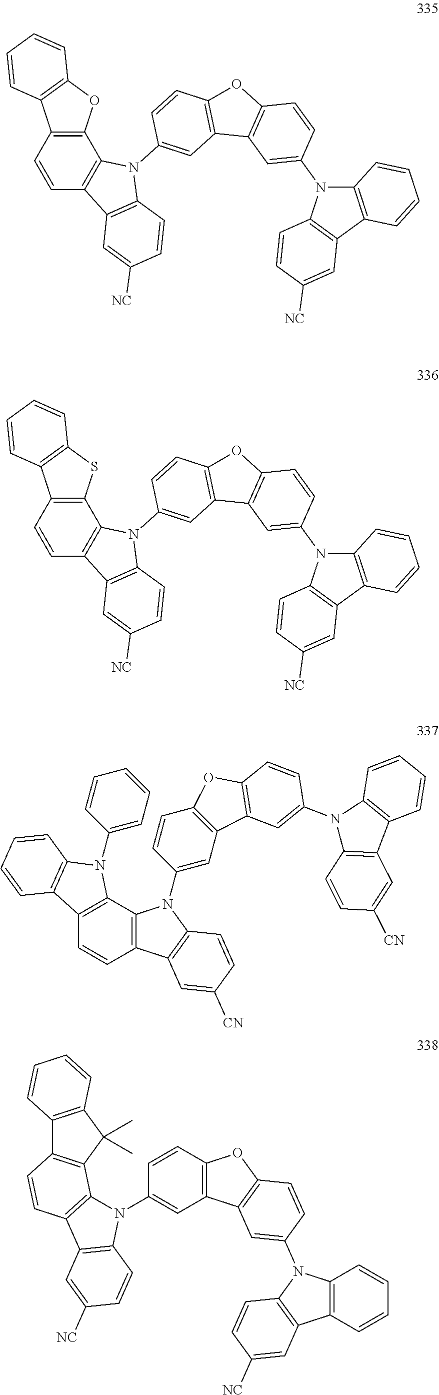 [SMILES: N#Cc1ccc2c(c1)c1ccccc1n2-c1ccc2oc3ccc(-n4c5ccc(C#N)cc5c5ccc6c7ccccc7oc6c54)cc3c2c1 ; N#Cc1ccc2c(c1)c1ccccc1n2-c1ccc2oc3ccc(-n4c5ccc(C#N)cc5c5ccc6c7ccccc7sc6c54)cc3c2c1 ; N#Cc1ccc2c(c1)c1ccccc1n2-c1ccc2oc3ccc(-n4c5ccc(C#N)cc5c5ccc6c7ccccc7n(-c7ccccc7)c6c54)cc3c2c1 ; CC1(C)c2ccccc2-c2ccc3c4cc(C#N)ccc4n(-c4ccc5oc6ccc(-n7c8ccccc8c8cc(C#N)ccc87)cc6c5c4)c3c21]

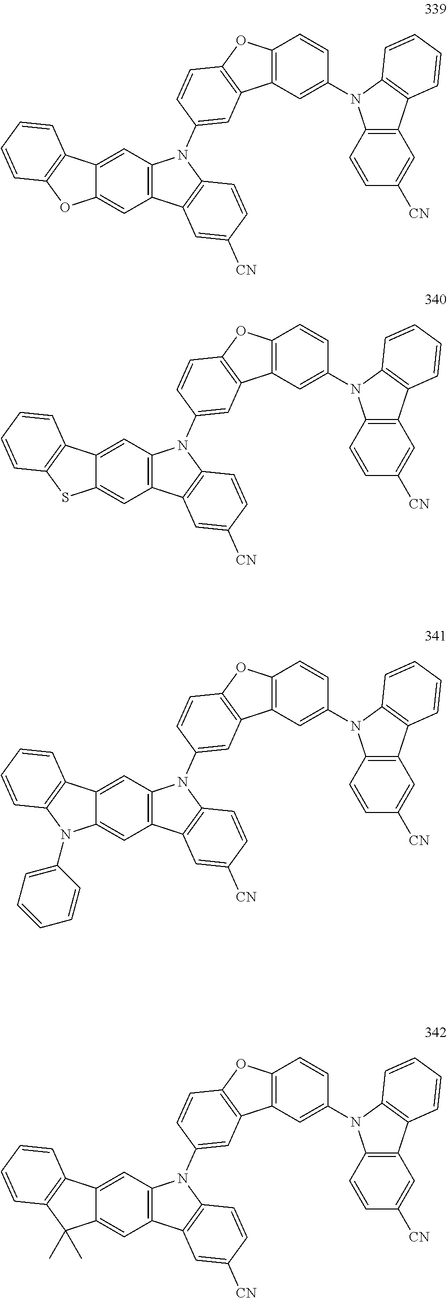 [SMILES: N#Cc1ccc2c(c1)c1ccccc1n2-c1ccc2oc3ccc(-n4c5ccc(C#N)cc5c5cc6oc7ccccc7c6cc54)cc3c2c1 ; N#Cc1ccc2c(c1)c1ccccc1n2-c1ccc2oc3ccc(-n4c5ccc(C#N)cc5c5cc6sc7ccccc7c6cc54)cc3c2c1 ; N#Cc1ccc2c(c1)c1ccccc1n2-c1ccc2oc3ccc(-n4c5ccc(C#N)cc5c5cc6c(cc54)c4ccccc4n6-c4ccccc4)cc3c2c1 ; CC1(C)c2ccccc2-c2cc3c(cc21)c1cc(C#N)ccc1n3-c1ccc2oc3ccc(-n4c5ccccc5c5cc(C#N)ccc54)cc3c2c1]

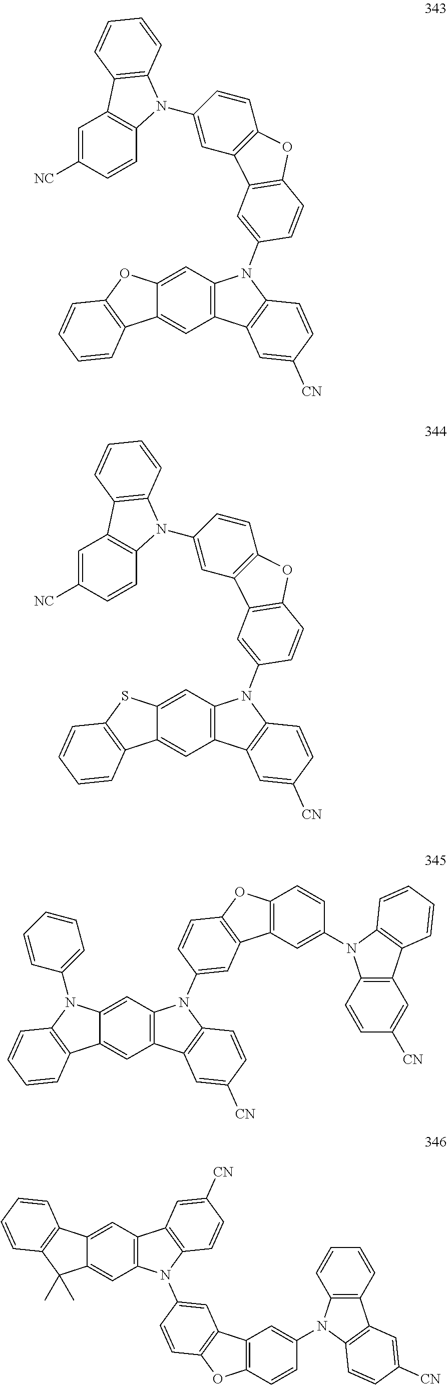 [SMILES: N#Cc1ccc2c(c1)c1ccccc1n2-c1ccc2oc3ccc(-n4c5ccc(C#N)cc5c5cc6c(cc54)oc4ccccc46)cc3c2c1 ; N#Cc1ccc2c(c1)c1ccccc1n2-c1ccc2oc3ccc(-n4c5ccc(C#N)cc5c5cc6c(cc54)sc4ccccc46)cc3c2c1 ; N#Cc1ccc2c(c1)c1ccccc1n2-c1ccc2oc3ccc(-n4c5ccc(C#N)cc5c5cc6c7ccccc7n(-c7ccccc7)c6cc54)cc3c2c1 ; CC1(C)c2ccccc2-c2cc3c4cc(C#N)ccc4n(-c4ccc5oc6ccc(-n7c8ccccc8c8cc(C#N)ccc87)cc6c5c4)c3cc21]

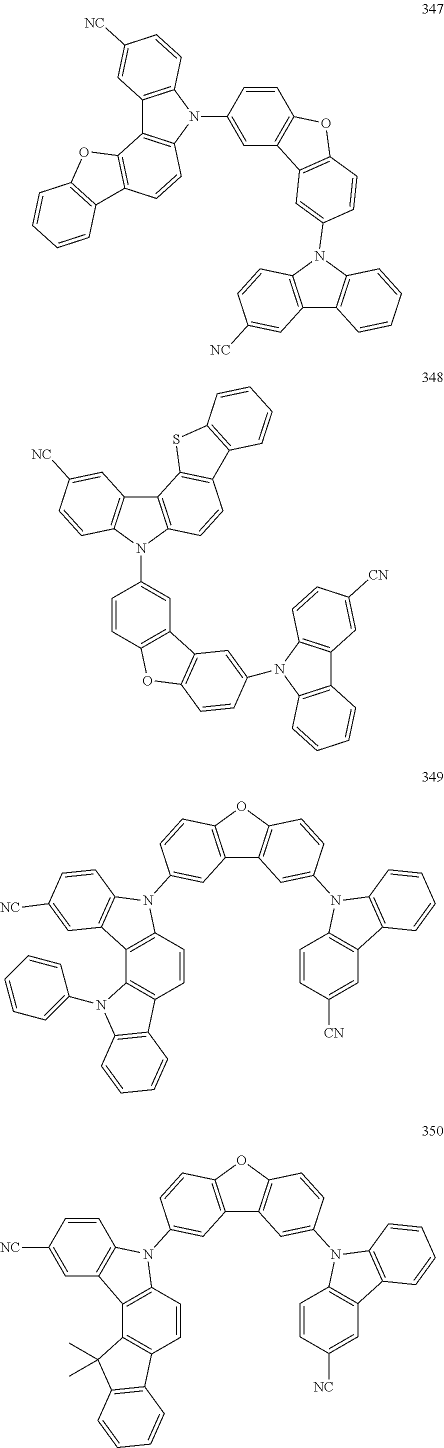

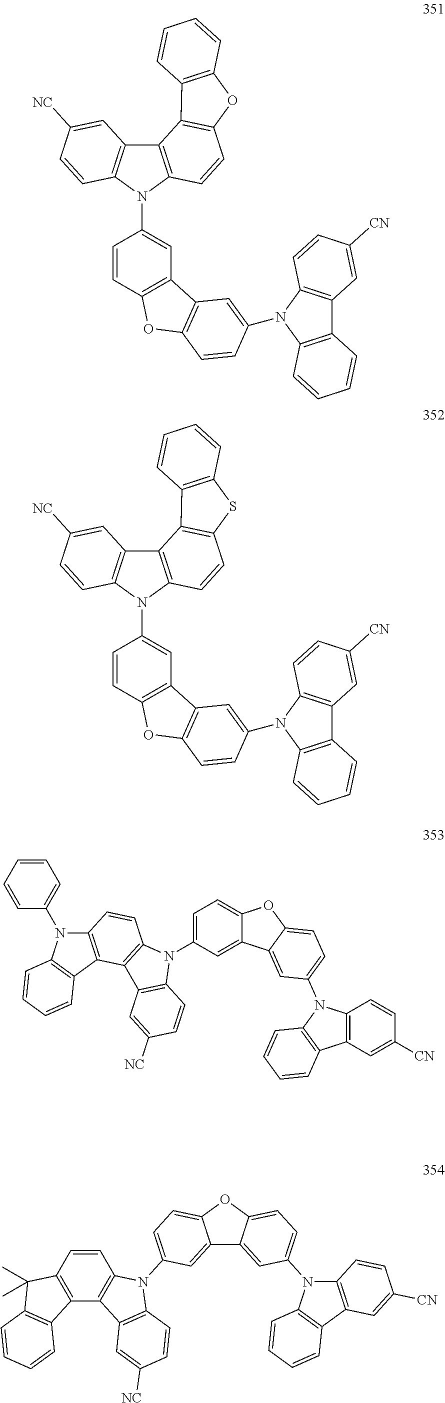 [SMILES: N#Cc1ccc2c(c1)c1ccccc1n2-c1ccc2oc3ccc(-n4c5ccc(C#N)cc5c5c6c(ccc54)oc4ccccc46)cc3c2c1 ; N#Cc1ccc2c(c1)c1ccccc1n2-c1ccc2oc3ccc(-n4c5ccc(C#N)cc5c5c6c(ccc54)sc4ccccc46)cc3c2c1 ; N#Cc1ccc2c(c1)c1ccccc1n2-c1ccc2oc3ccc(-n4c5ccc(C#N)cc5c5c6c7ccccc7n(-c7ccccc7)c6ccc54)cc3c2c1 ; CC1(C)c2ccccc2-c2c1ccc1c2c2cc(C#N)ccc2n1-c1ccc2oc3ccc(-n4c5ccccc5c5cc(C#N)ccc54)cc3c2c1]

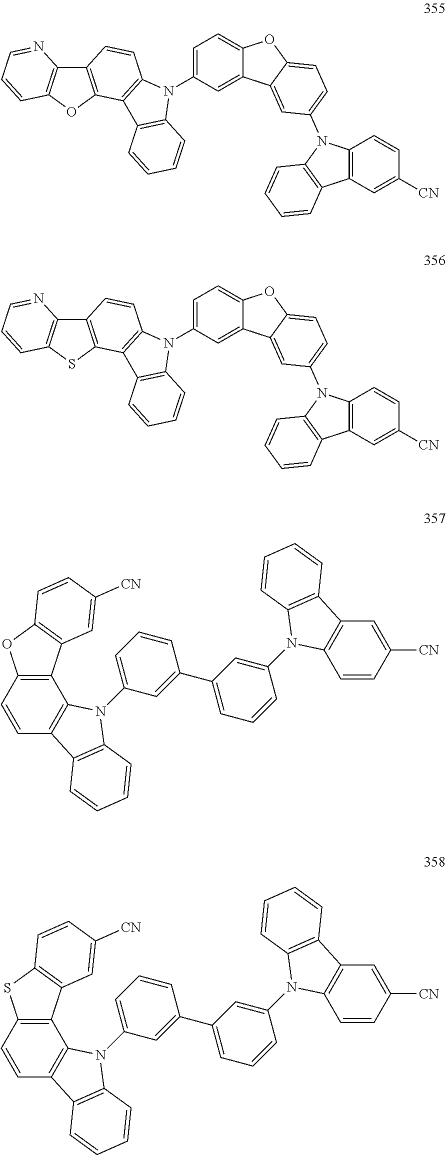 [SMILES: N#Cc1ccc2c(c1)c1ccccc1n2-c1ccc2oc3ccc(-n4c5ccccc5c5c6oc7cccnc7c6ccc54)cc3c2c1 ; N#Cc1ccc2c(c1)c1ccccc1n2-c1ccc2oc3ccc(-n4c5ccccc5c5c6sc7cccnc7c6ccc54)cc3c2c1 ; N#Cc1ccc2oc3ccc4c5ccccc5n(-c5cccc(-c6cccc(-n7c8ccccc8c8cc(C#N)ccc87)c6)c5)c4c3c2c1 ; N#Cc1ccc2sc3ccc4c5ccccc5n(-c5cccc(-c6cccc(-n7c8ccccc8c8cc(C#N)ccc87)c6)c5)c4c3c2c1]

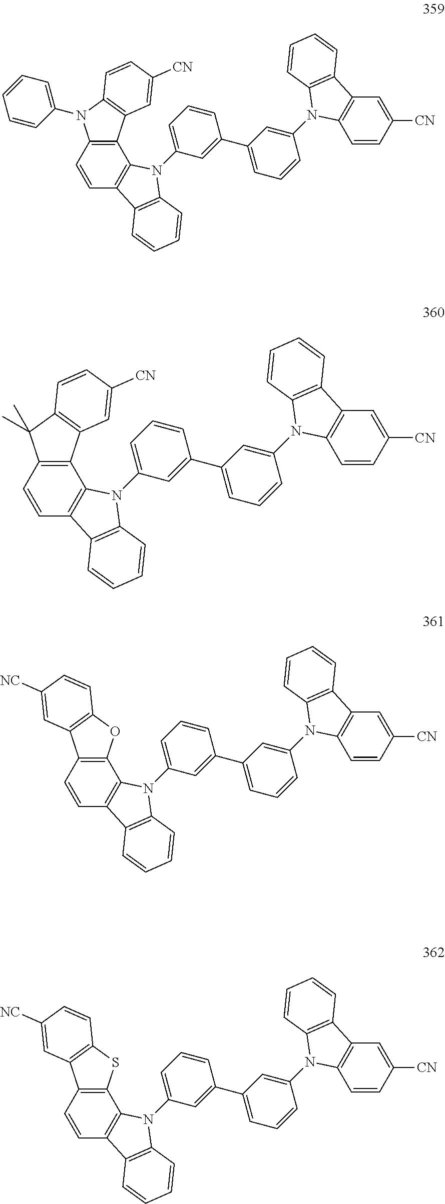 [SMILES: N#Cc1ccc2c(c1)c1ccccc1n2-c1cccc(-c2cccc(-n3c4ccccc4c4ccc5c(c6cc(C#N)ccc6n5-c5ccccc5)c43)c2)c1 ; CC1(C)c2ccc(C#N)cc2-c2c1ccc1c3ccccc3n(-c3cccc(-c4cccc(-n5c6ccccc6c6cc(C#N)ccc65)c4)c3)c21 ; N#Cc1ccc2oc3c(ccc4c5ccccc5n(-c5cccc(-c6cccc(-n7c8ccccc8c8cc(C#N)ccc87)c6)c5)c43)c2c1 ; N#Cc1ccc2sc3c(ccc4c5ccccc5n(-c5cccc(-c6cccc(-n7c8ccccc8c8cc(C#N)ccc87)c6)c5)c43)c2c1]

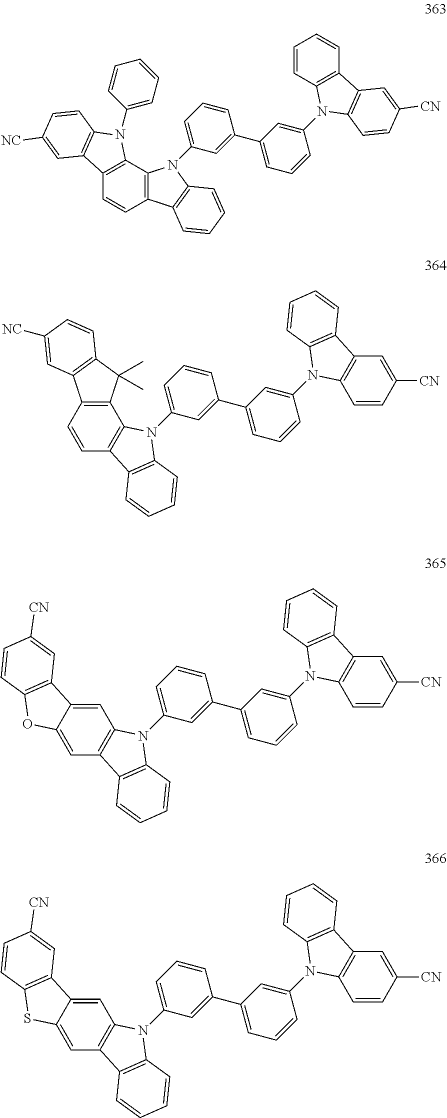 [SMILES: N#Cc1ccc2c(c1)c1ccccc1n2-c1cccc(-c2cccc(-n3c4ccccc4c4ccc5c6cc(C#N)ccc6n(-c6ccccc6)c5c43)c2)c1 ; CC1(C)c2ccc(C#N)cc2-c2ccc3c4ccccc4n(-c4cccc(-c5cccc(-n6c7ccccc7c7cc(C#N)ccc76)c5)c4)c3c21 ; N#Cc1ccc2oc3cc4c5ccccc5n(-c5cccc(-c6cccc(-n7c8ccccc8c8cc(C#N)ccc87)c6)c5)c4cc3c2c1 ; N#Cc1ccc2sc3cc4c5ccccc5n(-c5cccc(-c6cccc(-n7c8ccccc8c8cc(C#N)ccc87)c6)c5)c4cc3c2c1]

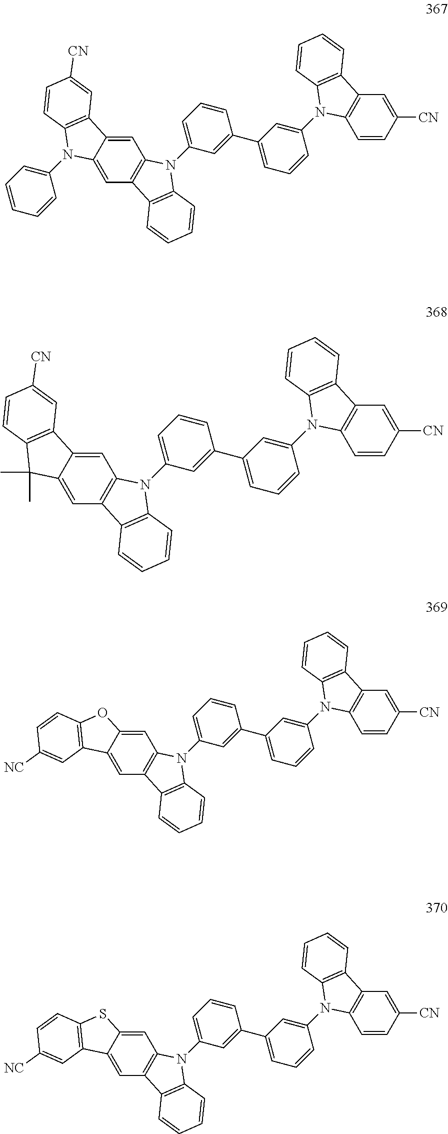 [SMILES: N#Cc1ccc2c(c1)c1ccccc1n2-c1cccc(-c2cccc(-n3c4ccccc4c4cc5c(cc43)c3cc(C#N)ccc3n5-c3ccccc3)c2)c1 ; CC1(C)c2ccc(C#N)cc2-c2cc3c(cc21)c1ccccc1n3-c1cccc(-c2cccc(-n3c4ccccc4c4cc(C#N)ccc43)c2)c1 ; N#Cc1ccc2oc3cc4c(cc3c2c1)c1ccccc1n4-c1cccc(-c2cccc(-n3c4ccccc4c4cc(C#N)ccc43)c2)c1 ; N#Cc1ccc2sc3cc4c(cc3c2c1)c1ccccc1n4-c1cccc(-c2cccc(-n3c4ccccc4c4cc(C#N)ccc43)c2)c1]

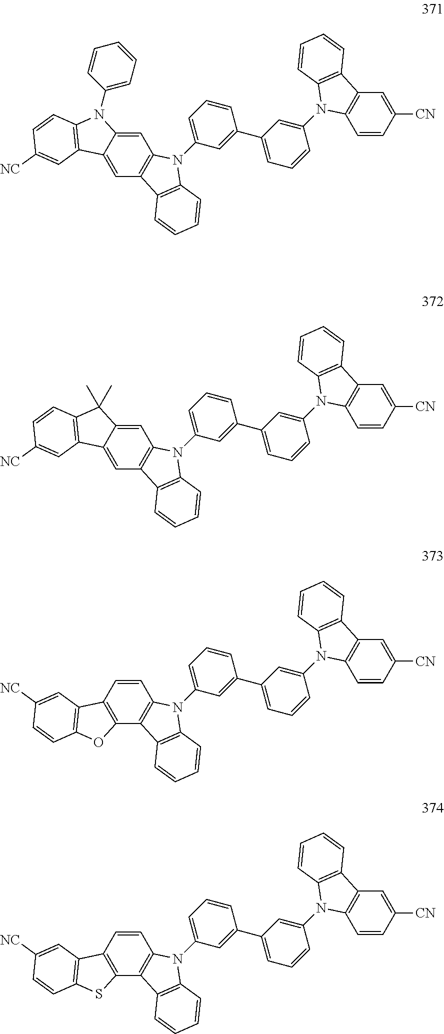 [SMILES: N#Cc1ccc2c(c1)c1ccccc1n2-c1cccc(-c2cccc(-n3c4ccccc4c4cc5c6cc(C#N)ccc6n(-c6ccccc6)c5cc43)c2)c1 ; CC1(C)c2ccc(C#N)cc2-c2cc3c4ccccc4n(-c4cccc(-c5cccc(-n6c7ccccc7c7cc(C#N)ccc76)c5)c4)c3cc21 ; N#Cc1ccc2oc3c(ccc4c3c3ccccc3n4-c3cccc(-c4cccc(-n5c6ccccc6c6cc(C#N)ccc65)c4)c3)c2c1 ; N#Cc1ccc2sc3c(ccc4c3c3ccccc3n4-c3cccc(-c4cccc(-n5c6ccccc6c6cc(C#N)ccc65)c4)c3)c2c1]

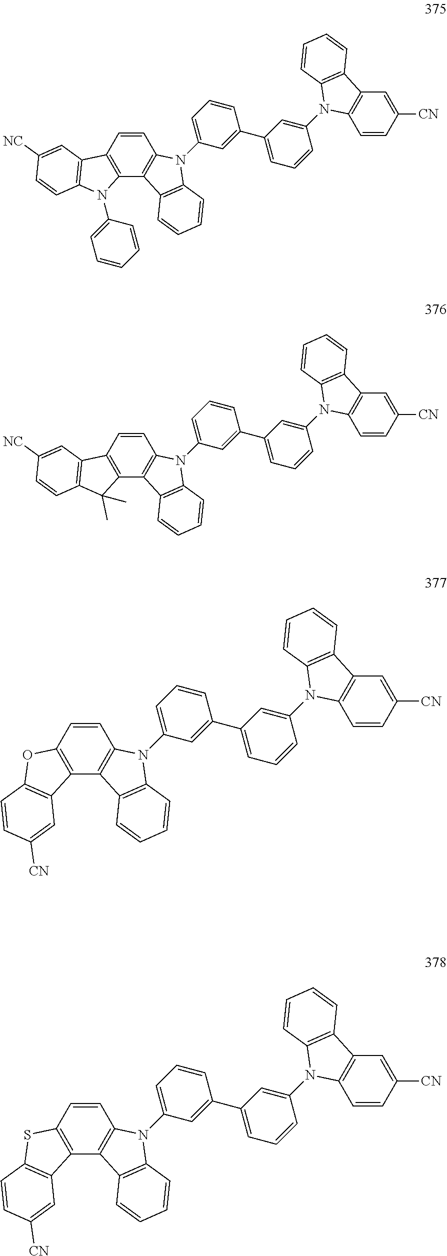 [SMILES: N#Cc1ccc2c(c1)c1ccccc1n2-c1cccc(-c2cccc(-n3c4ccccc4c4c3ccc3c5cc(C#N)ccc5n(-c5ccccc5)c34)c2)c1 ; CC1(C)c2ccc(C#N)cc2-c2ccc3c(c21)c1ccccc1n3-c1cccc(-c2cccc(-n3c4ccccc4c4cc(C#N)ccc43)c2)c1 ; N#Cc1ccc2oc3ccc4c(c5ccccc5n4-c4cccc(-c5cccc(-n6c7ccccc7c7cc(C#N)ccc76)c5)c4)c3c2c1 ; N#Cc1ccc2sc3ccc4c(c5ccccc5n4-c4cccc(-c5cccc(-n6c7ccccc7c7cc(C#N)ccc76)c5)c4)c3c2c1]

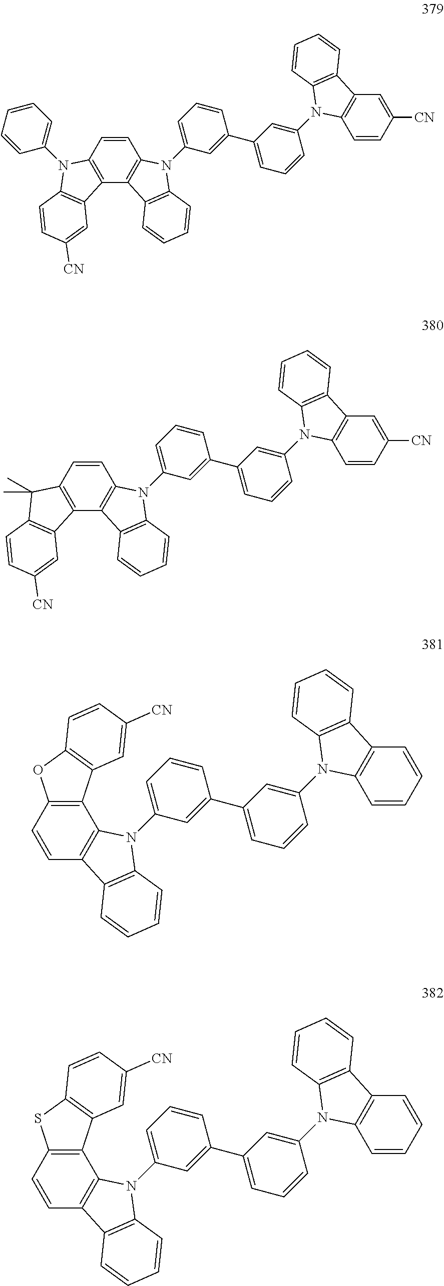 [SMILES: N#Cc1ccc2c(c1)c1ccccc1n2-c1cccc(-c2cccc(-n3c4ccccc4c4c5c6cc(C#N)ccc6n(-c6ccccc6)c5ccc43)c2)c1 ; CC1(C)c2ccc(C#N)cc2-c2c1ccc1c2c2ccccc2n1-c1cccc(-c2cccc(-n3c4ccccc4c4cc(C#N)ccc43)c2)c1 ; N#Cc1ccc2oc3ccc4c5ccccc5n(-c5cccc(-c6cccc(-n7c8ccccc8c8ccccc87)c6)c5)c4c3c2c1 ; N#Cc1ccc2sc3ccc4c5ccccc5n(-c5cccc(-c6cccc(-n7c8ccccc8c8ccccc87)c6)c5)c4c3c2c1]

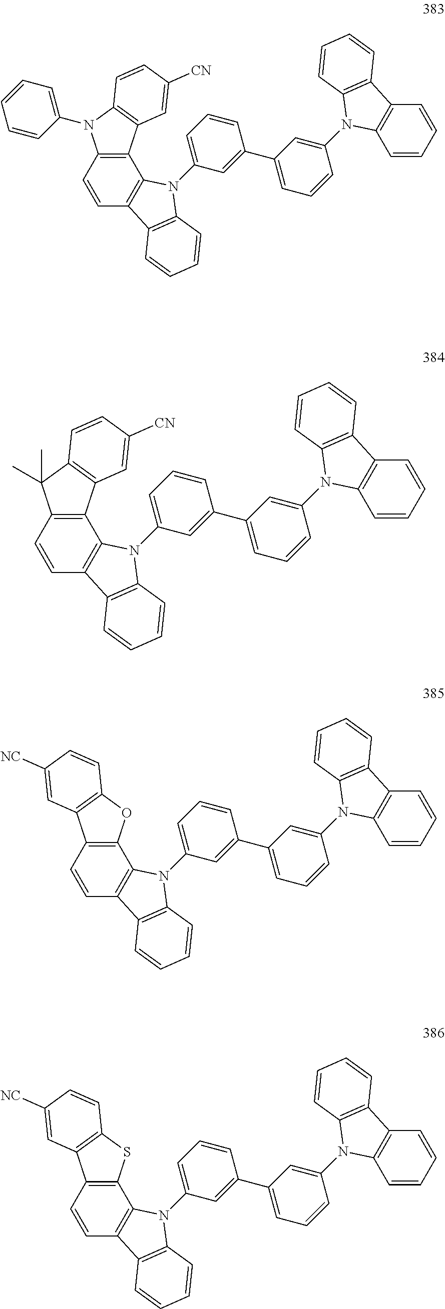 [SMILES: N#Cc1ccc2c(c1)c1c(ccc3c4ccccc4n(-c4cccc(-c5cccc(-n6c7ccccc7c7ccccc76)c5)c4)c31)n2-c1ccccc1 ; CC1(C)c2ccc(C#N)cc2-c2c1ccc1c3ccccc3n(-c3cccc(-c4cccc(-n5c6ccccc6c6ccccc65)c4)c3)c21 ; N#Cc1ccc2oc3c(ccc4c5ccccc5n(-c5cccc(-c6cccc(-n7c8ccccc8c8ccccc87)c6)c5)c43)c2c1 ; N#Cc1ccc2sc3c(ccc4c5ccccc5n(-c5cccc(-c6cccc(-n7c8ccccc8c8ccccc87)c6)c5)c43)c2c1]

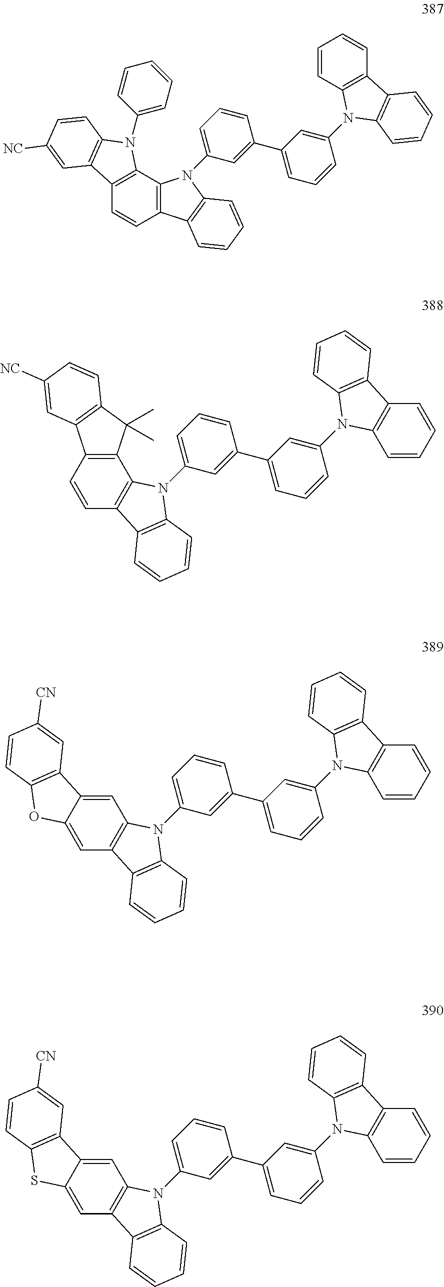 [SMILES: N#Cc1ccc2c(c1)c1ccc3c4ccccc4n(-c4cccc(-c5cccc(-n6c7ccccc7c7ccccc76)c5)c4)c3c1n2-c1ccccc1 ; CC1(C)c2ccc(C#N)cc2-c2ccc3c4ccccc4n(-c4cccc(-c5cccc(-n6c7ccccc7c7ccccc76)c5)c4)c3c21 ; N#Cc1ccc2oc3cc4c5ccccc5n(-c5cccc(-c6cccc(-n7c8ccccc8c8ccccc87)c6)c5)c4cc3c2c1 ; N#Cc1ccc2sc3cc4c5ccccc5n(-c5cccc(-c6cccc(-n7c8ccccc8c8ccccc87)c6)c5)c4cc3c2c1]

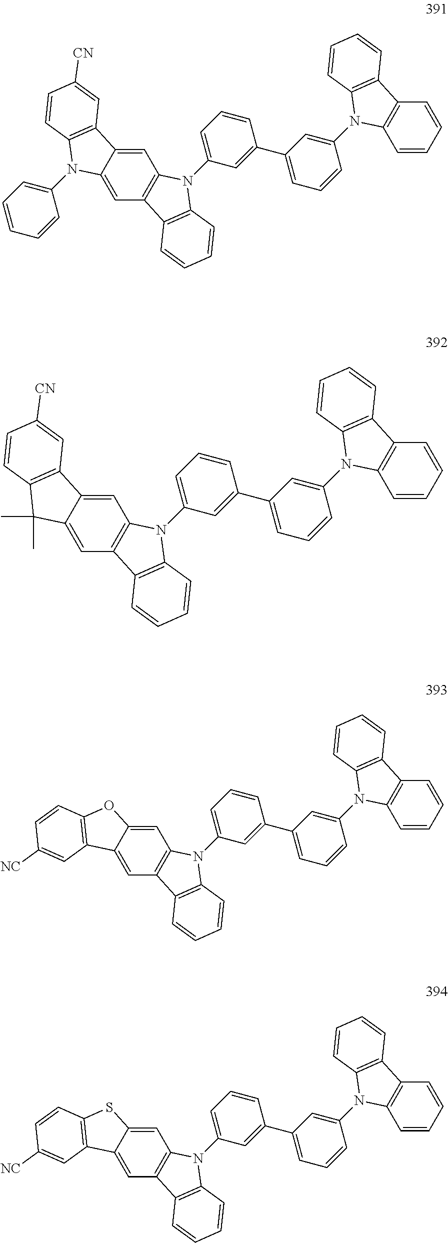 [SMILES: N#Cc1ccc2c(c1)c1cc3c(cc1n2-c1ccccc1)c1ccccc1n3-c1cccc(-c2cccc(-n3c4ccccc4c4ccccc43)c2)c1 ; CC1(C)c2ccc(C#N)cc2-c2cc3c(cc21)c1ccccc1n3-c1cccc(-c2cccc(-n3c4ccccc4c4ccccc43)c2)c1 ; N#Cc1ccc2oc3cc4c(cc3c2c1)c1ccccc1n4-c1cccc(-c2cccc(-n3c4ccccc4c4ccccc43)c2)c1 ; N#Cc1ccc2sc3cc4c(cc3c2c1)c1ccccc1n4-c1cccc(-c2cccc(-n3c4ccccc4c4ccccc43)c2)c1]

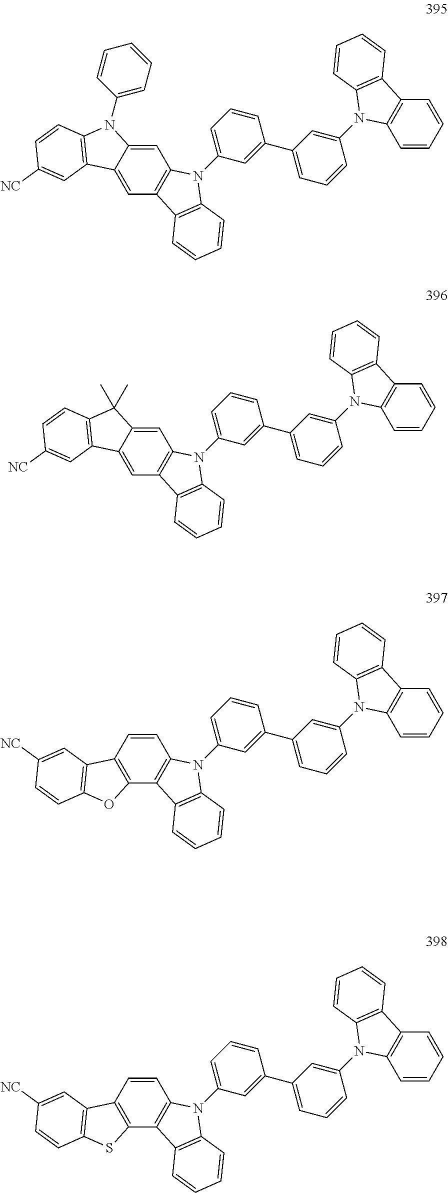 [SMILES: N#Cc1ccc2c(c1)c1cc3c4ccccc4n(-c4cccc(-c5cccc(-n6c7ccccc7c7ccccc76)c5)c4)c3cc1n2-c1ccccc1 ; CC1(C)c2ccc(C#N)cc2-c2cc3c4ccccc4n(-c4cccc(-c5cccc(-n6c7ccccc7c7ccccc76)c5)c4)c3cc21 ; N#Cc1ccc2oc3c(ccc4c3c3ccccc3n4-c3cccc(-c4cccc(-n5c6ccccc6c6ccccc65)c4)c3)c2c1 ; N#Cc1ccc2sc3c(ccc4c3c3ccccc3n4-c3cccc(-c4cccc(-n5c6ccccc6c6ccccc65)c4)c3)c2c1]

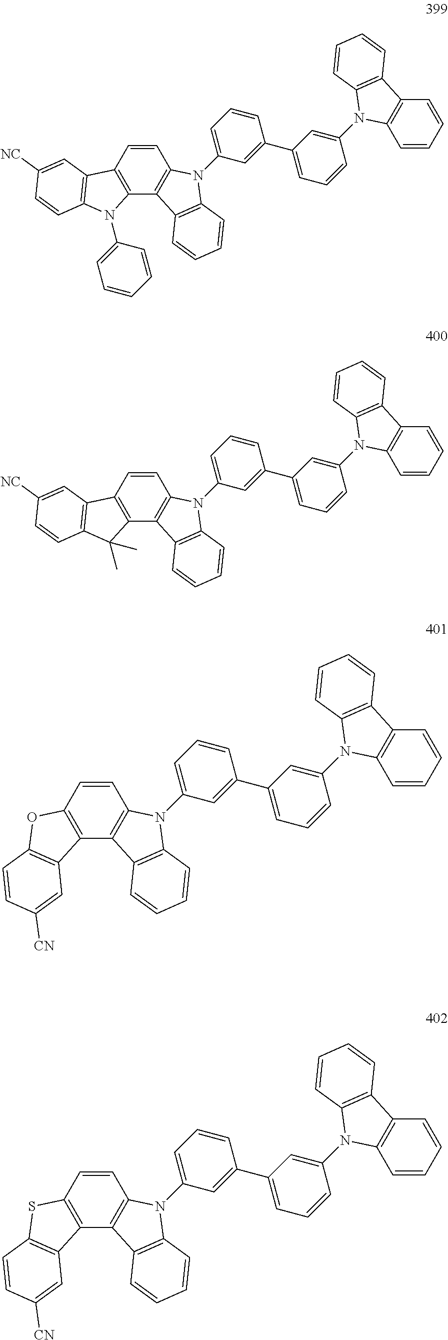 [SMILES: N#Cc1ccc2c(c1)c1ccc3c(c4ccccc4n3-c3cccc(-c4cccc(-n5c6ccccc6c6ccccc65)c4)c3)c1n2-c1ccccc1 ; CC1(C)c2ccc(C#N)cc2-c2ccc3c(c21)c1ccccc1n3-c1cccc(-c2cccc(-n3c4ccccc4c4ccccc43)c2)c1 ; N#Cc1ccc2oc3ccc4c(c5ccccc5n4-c4cccc(-c5cccc(-n6c7ccccc7c7ccccc76)c5)c4)c3c2c1 ; N#Cc1ccc2sc3ccc4c(c5ccccc5n4-c4cccc(-c5cccc(-n6c7ccccc7c7ccccc76)c5)c4)c3c2c1]

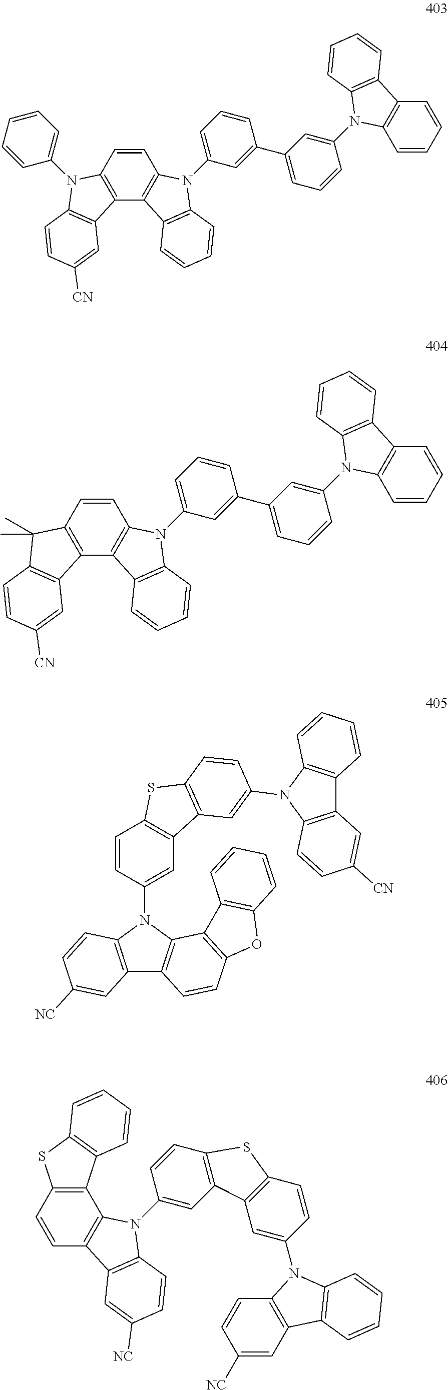 [SMILES: N#Cc1ccc2c(c1)c1c3c4ccccc4n(-c4cccc(-c5cccc(-n6c7ccccc7c7ccccc76)c5)c4)c3ccc1n2-c1ccccc1 ; CC1(C)c2ccc(C#N)cc2-c2c1ccc1c2c2ccccc2n1-c1cccc(-c2cccc(-n3c4ccccc4c4ccccc43)c2)c1 ; N#Cc1ccc2c(c1)c1ccccc1n2-c1ccc2sc3ccc(-n4c5ccc(C#N)cc5c5ccc6oc7ccccc7c6c54)cc3c2c1 ; N#Cc1ccc2c(c1)c1ccccc1n2-c1ccc2sc3ccc(-n4c5ccc(C#N)cc5c5ccc6sc7ccccc7c6c54)cc3c2c1]

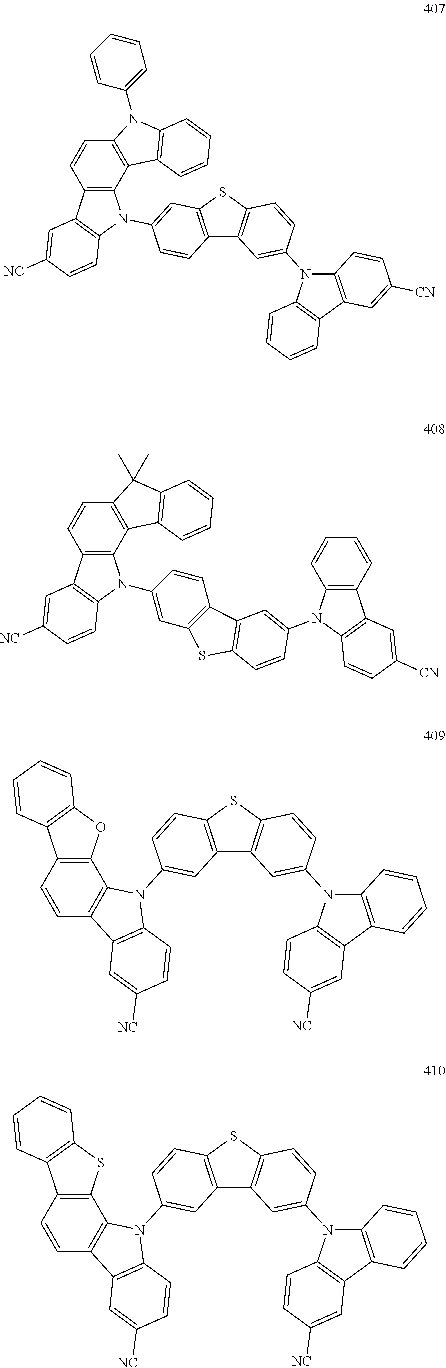 [SMILES: N#Cc1ccc2c(c1)c1ccccc1n2-c1ccc2sc3cc(-n4c5ccc(C#N)cc5c5ccc6c(c7ccccc7n6-c6ccccc6)c54)ccc3c2c1 ; CC1(C)c2ccccc2-c2c1ccc1c3cc(C#N)ccc3n(-c3ccc4c(c3)sc3ccc(-n5c6ccccc6c6cc(C#N)ccc65)cc34)c21 ; N#Cc1ccc2c(c1)c1ccccc1n2-c1ccc2sc3ccc(-n4c5ccc(C#N)cc5c5ccc6c7ccccc7oc6c54)cc3c2c1 ; N#Cc1ccc2c(c1)c1ccccc1n2-c1ccc2sc3ccc(-n4c5ccc(C#N)cc5c5ccc6c7ccccc7sc6c54)cc3c2c1]

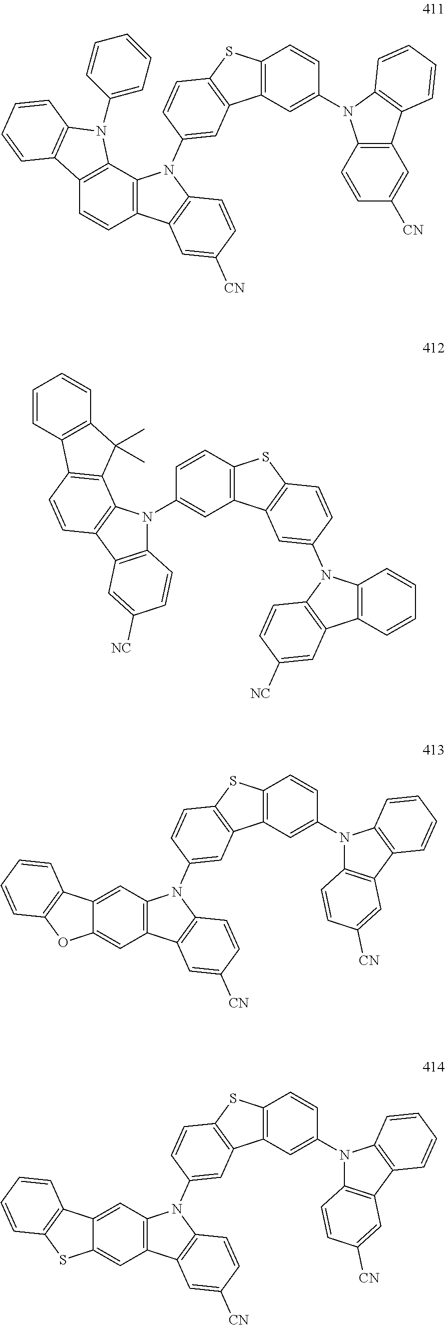 [SMILES: N#Cc1ccc2c(c1)c1ccccc1n2-c1ccc2sc3ccc(-n4c5ccc(C#N)cc5c5ccc6c7ccccc7n(-c7ccccc7)c6c54)cc3c2c1 ; CC1(C)c2ccccc2-c2ccc3c4cc(C#N)ccc4n(-c4ccc5sc6ccc(-n7c8ccccc8c8cc(C#N)ccc87)cc6c5c4)c3c21 ; N#Cc1ccc2c(c1)c1ccccc1n2-c1ccc2sc3ccc(-n4c5ccc(C#N)cc5c5cc6oc7ccccc7c6cc54)cc3c2c1 ; N#Cc1ccc2c(c1)c1ccccc1n2-c1ccc2sc3ccc(-n4c5ccc(C#N)cc5c5cc6sc7ccccc7c6cc54)cc3c2c1]

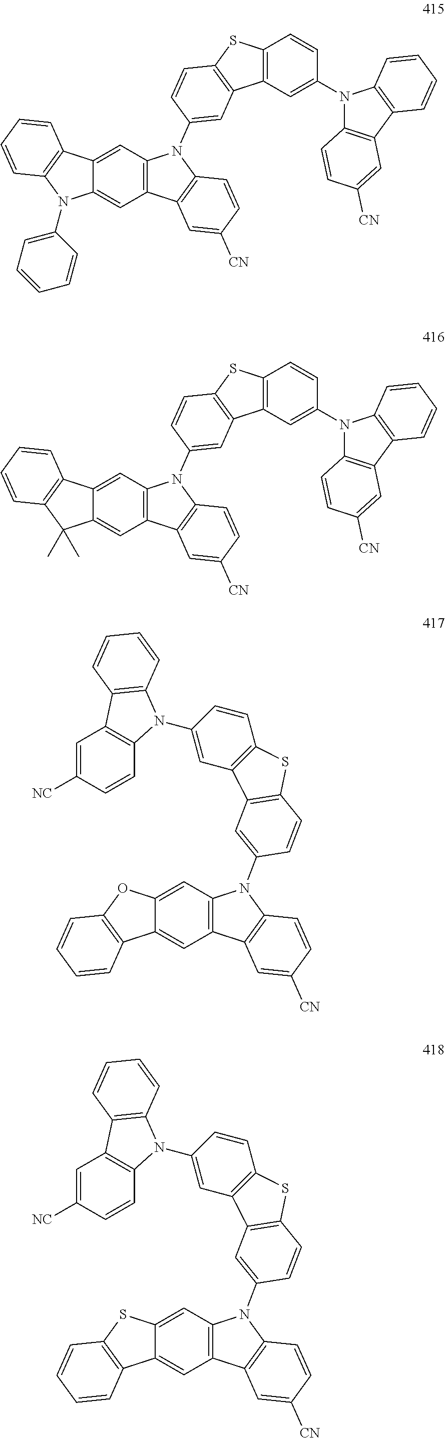 [SMILES: N#Cc1ccc2c(c1)c1ccccc1n2-c1ccc2sc3ccc(-n4c5ccc(C#N)cc5c5cc6c(cc54)c4ccccc4n6-c4ccccc4)cc3c2c1 ; CC1(C)c2ccccc2-c2cc3c(cc21)c1cc(C#N)ccc1n3-c1ccc2sc3ccc(-n4c5ccccc5c5cc(C#N)ccc54)cc3c2c1 ; N#Cc1ccc2c(c1)c1ccccc1n2-c1ccc2sc3ccc(-n4c5ccc(C#N)cc5c5cc6c(cc54)oc4ccccc46)cc3c2c1 ; N#Cc1ccc2c(c1)c1ccccc1n2-c1ccc2sc3ccc(-n4c5ccc(C#N)cc5c5cc6c(cc54)sc4ccccc46)cc3c2c1]

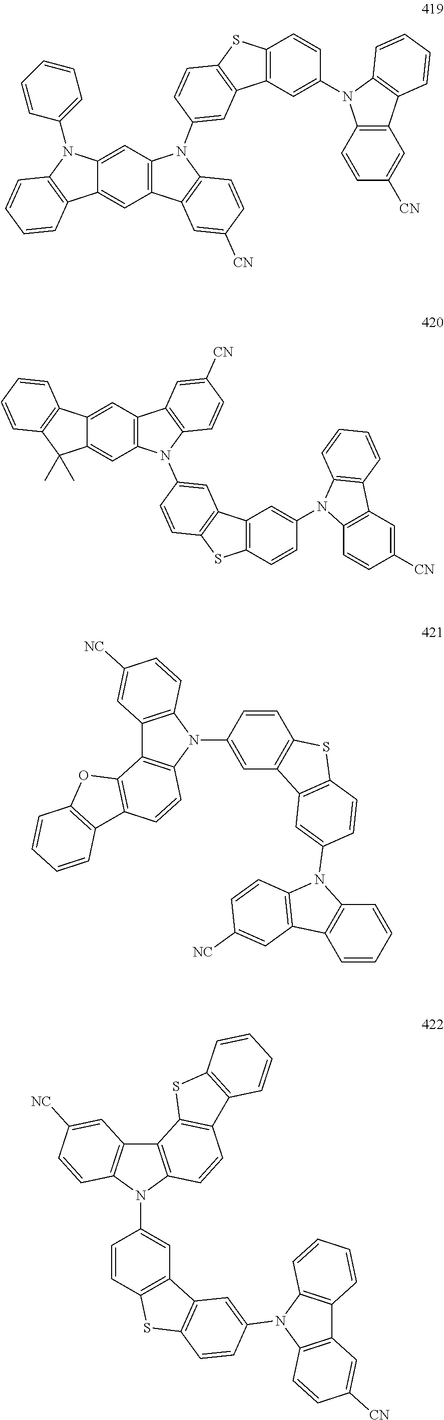 [SMILES: N#Cc1ccc2c(c1)c1ccccc1n2-c1ccc2sc3ccc(-n4c5ccc(C#N)cc5c5cc6c7ccccc7n(-c7ccccc7)c6cc54)cc3c2c1 ; CC1(C)c2ccccc2-c2cc3c4cc(C#N)ccc4n(-c4ccc5sc6ccc(-n7c8ccccc8c8cc(C#N)ccc87)cc6c5c4)c3cc21 ; N#Cc1ccc2c(c1)c1ccccc1n2-c1ccc2sc3ccc(-n4c5ccc(C#N)cc5c5c6oc7ccccc7c6ccc54)cc3c2c1 ; N#Cc1ccc2c(c1)c1ccccc1n2-c1ccc2sc3ccc(-n4c5ccc(C#N)cc5c5c6sc7ccccc7c6ccc54)cc3c2c1]

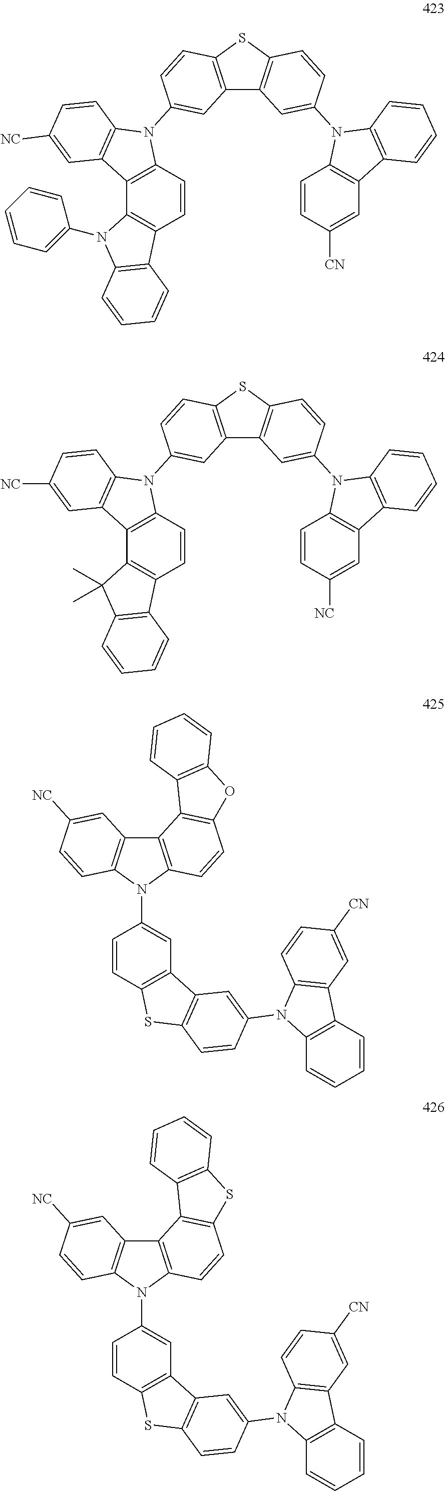 [SMILES: N#Cc1ccc2c(c1)c1ccccc1n2-c1ccc2sc3ccc(-n4c5ccc(C#N)cc5c5c4ccc4c6ccccc6n(-c6ccccc6)c45)cc3c2c1 ; CC1(C)c2ccccc2-c2ccc3c(c21)c1cc(C#N)ccc1n3-c1ccc2sc3ccc(-n4c5ccccc5c5cc(C#N)ccc54)cc3c2c1 ; N#Cc1ccc2c(c1)c1ccccc1n2-c1ccc2sc3ccc(-n4c5ccc(C#N)cc5c5c6c(ccc54)oc4ccccc46)cc3c2c1 ; N#Cc1ccc2c(c1)c1ccccc1n2-c1ccc2sc3ccc(-n4c5ccc(C#N)cc5c5c6c(ccc54)sc4ccccc46)cc3c2c1]

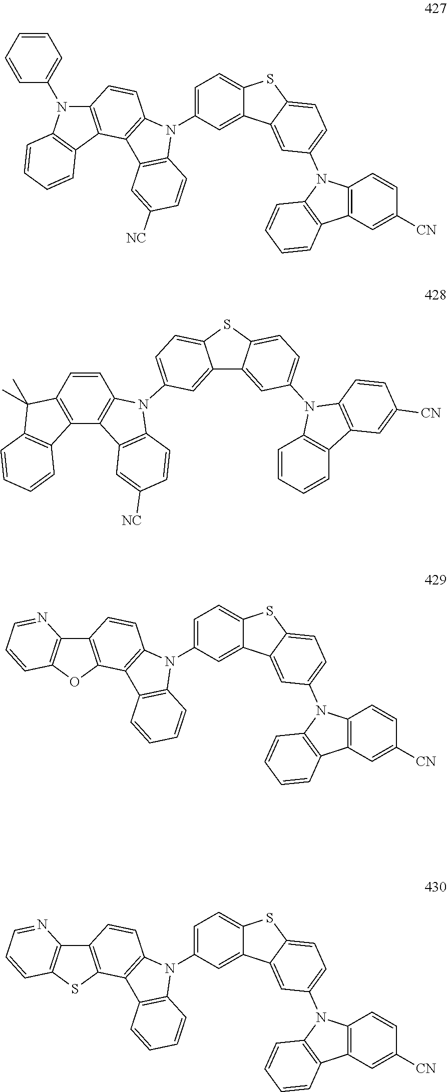 [SMILES: N#Cc1ccc2c(c1)c1ccccc1n2-c1ccc2sc3ccc(-n4c5ccc(C#N)cc5c5c6c7ccccc7n(-c7ccccc7)c6ccc54)cc3c2c1 ; CC1(C)c2ccccc2-c2c1ccc1c2c2cc(C#N)ccc2n1-c1ccc2sc3ccc(-n4c5ccccc5c5cc(C#N)ccc54)cc3c2c1 ; N#Cc1ccc2c(c1)c1ccccc1n2-c1ccc2sc3ccc(-n4c5ccccc5c5c6oc7cccnc7c6ccc54)cc3c2c1 ; N#Cc1ccc2c(c1)c1ccccc1n2-c1ccc2sc3ccc(-n4c5ccccc5c5c6sc7cccnc7c6ccc54)cc3c2c1]

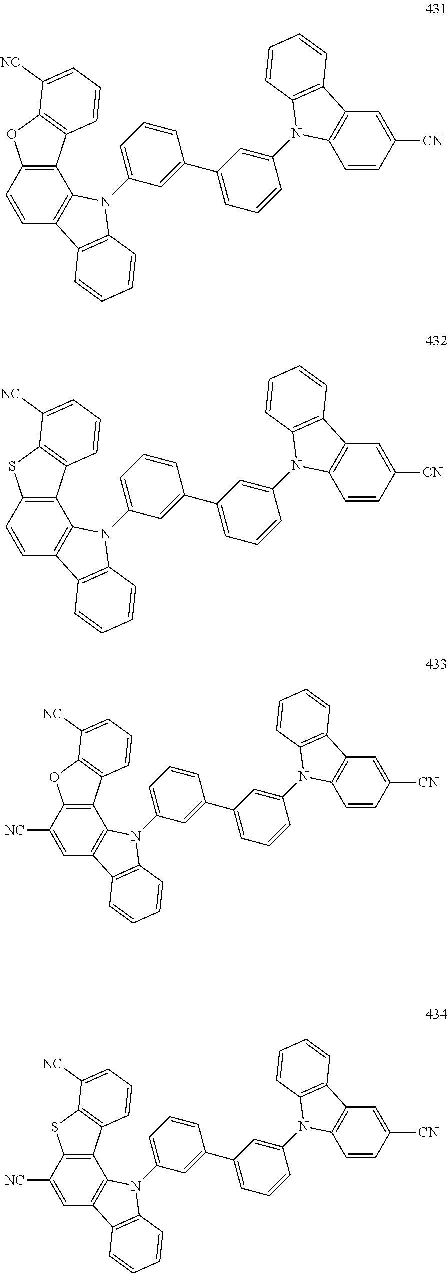 [SMILES: N#Cc1ccc2c(c1)c1ccccc1n2-c1cccc(-c2cccc(-n3c4ccccc4c4ccc5oc6c(C#N)cccc6c5c43)c2)c1 ; N#Cc1ccc2c(c1)c1ccccc1n2-c1cccc(-c2cccc(-n3c4ccccc4c4ccc5sc6c(C#N)cccc6c5c43)c2)c1 ; N#Cc1ccc2c(c1)c1ccccc1n2-c1cccc(-c2cccc(-n3c4ccccc4c4cc(C#N)c5oc6c(C#N)cccc6c5c43)c2)c1 ; N#Cc1ccc2c(c1)c1ccccc1n2-c1cccc(-c2cccc(-n3c4ccccc4c4cc(C#N)c5sc6c(C#N)cccc6c5c43)c2)c1]

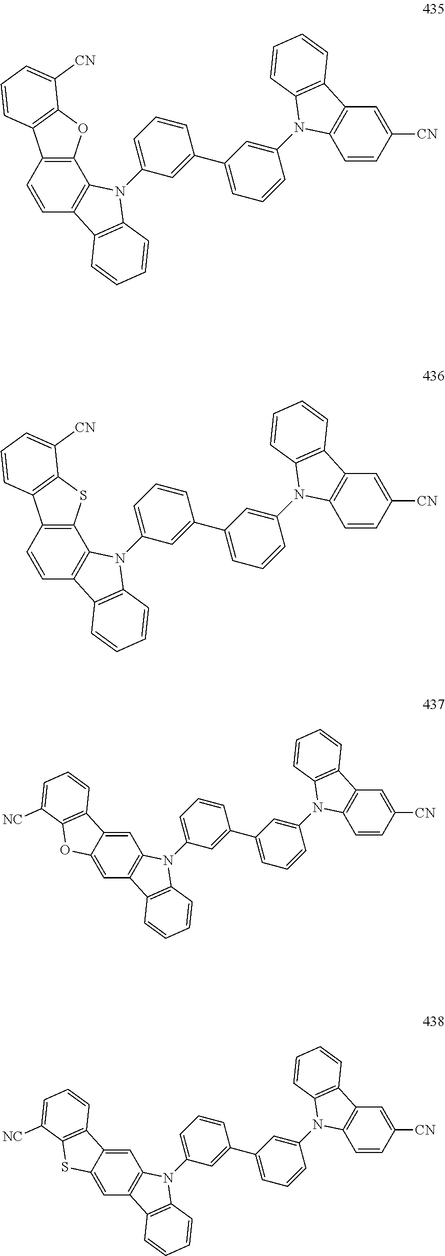 [SMILES: N#Cc1ccc2c(c1)c1ccccc1n2-c1cccc(-c2cccc(-n3c4ccccc4c4ccc5c6cccc(C#N)c6oc5c43)c2)c1 ; N#Cc1ccc2c(c1)c1ccccc1n2-c1cccc(-c2cccc(-n3c4ccccc4c4ccc5c6cccc(C#N)c6sc5c43)c2)c1 ; N#Cc1ccc2c(c1)c1ccccc1n2-c1cccc(-c2cccc(-n3c4ccccc4c4cc5oc6c(C#N)cccc6c5cc43)c2)c1 ; N#Cc1ccc2c(c1)c1ccccc1n2-c1cccc(-c2cccc(-n3c4ccccc4c4cc5sc6c(C#N)cccc6c5cc43)c2)c1]

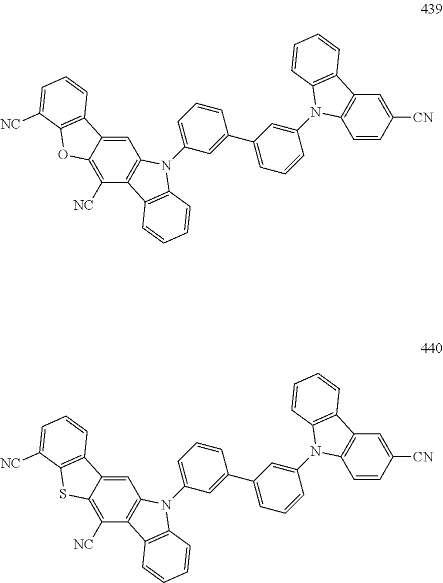 [SMILES: N#Cc1ccc2c(c1)c1ccccc1n2-c1cccc(-c2cccc(-n3c4ccccc4c4c(C#N)c5oc6c(C#N)cccc6c5cc43)c2)c1 ; N#Cc1ccc2c(c1)c1ccccc1n2-c1cccc(-c2cccc(-n3c4ccccc4c4c(C#N)c5sc6c(C#N)cccc6c5cc43)c2)c1]

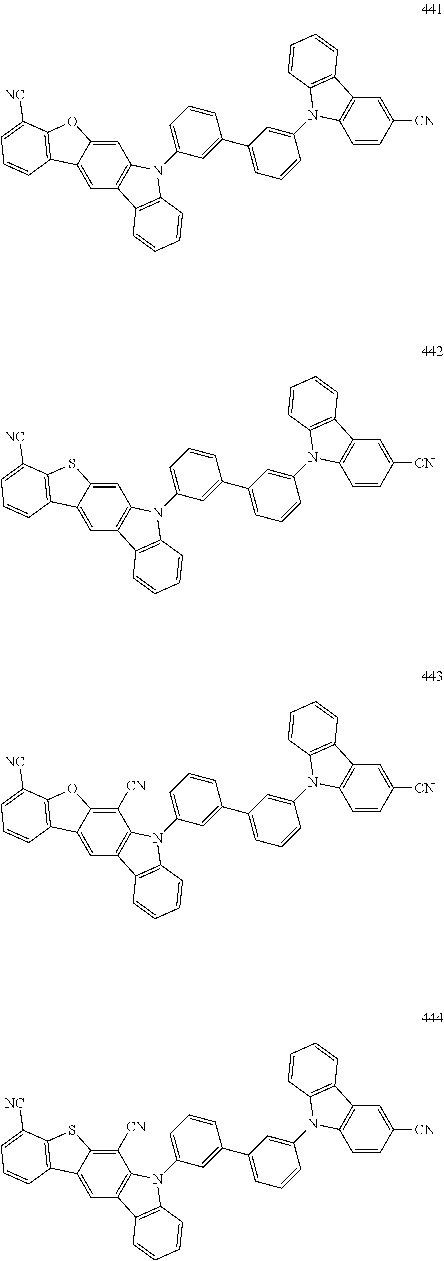 [SMILES: N#Cc1ccc2c(c1)c1ccccc1n2-c1cccc(-c2cccc(-n3c4ccccc4c4cc5c(cc43)oc3c(C#N)cccc35)c2)c1 ; N#Cc1ccc2c(c1)c1ccccc1n2-c1cccc(-c2cccc(-n3c4ccccc4c4cc5c(cc43)sc3c(C#N)cccc35)c2)c1 ; N#Cc1ccc2c(c1)c1ccccc1n2-c1cccc(-c2cccc(-n3c4ccccc4c4cc5c(oc6c(C#N)cccc65)c(C#N)c43)c2)c1 ; N#Cc1ccc2c(c1)c1ccccc1n2-c1cccc(-c2cccc(-n3c4ccccc4c4cc5c(sc6c(C#N)cccc65)c(C#N)c43)c2)c1]

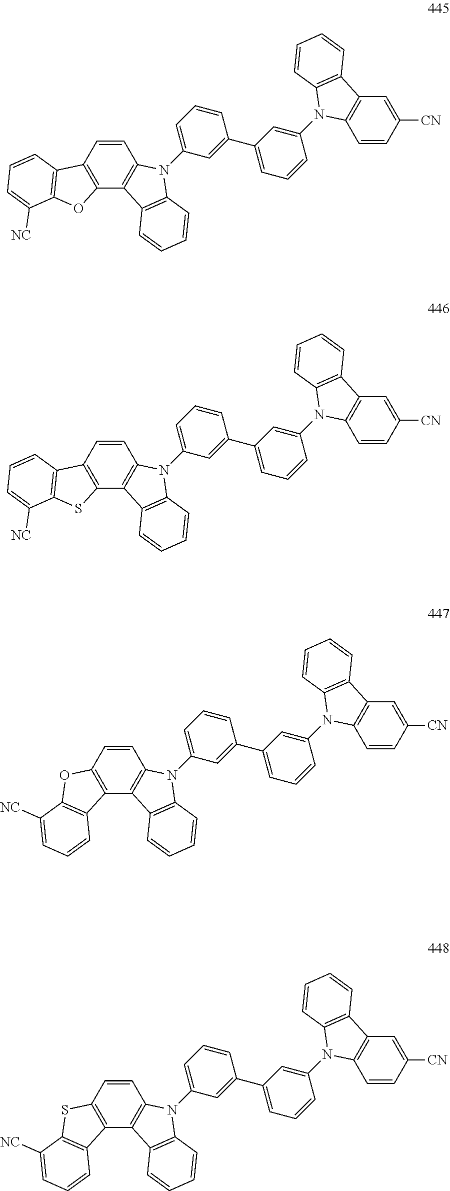 [SMILES: N#Cc1ccc2c(c1)c1ccccc1n2-c1cccc(-c2cccc(-n3c4ccccc4c4c5oc6c(C#N)cccc6c5ccc43)c2)c1 ; N#Cc1ccc2c(c1)c1ccccc1n2-c1cccc(-c2cccc(-n3c4ccccc4c4c5sc6c(C#N)cccc6c5ccc43)c2)c1 ; N#Cc1ccc2c(c1)c1ccccc1n2-c1cccc(-c2cccc(-n3c4ccccc4c4c5c(ccc43)oc3c(C#N)cccc35)c2)c1 ; N#Cc1ccc2c(c1)c1ccccc1n2-c1cccc(-c2cccc(-n3c4ccccc4c4c5c(ccc43)sc3c(C#N)cccc35)c2)c1]

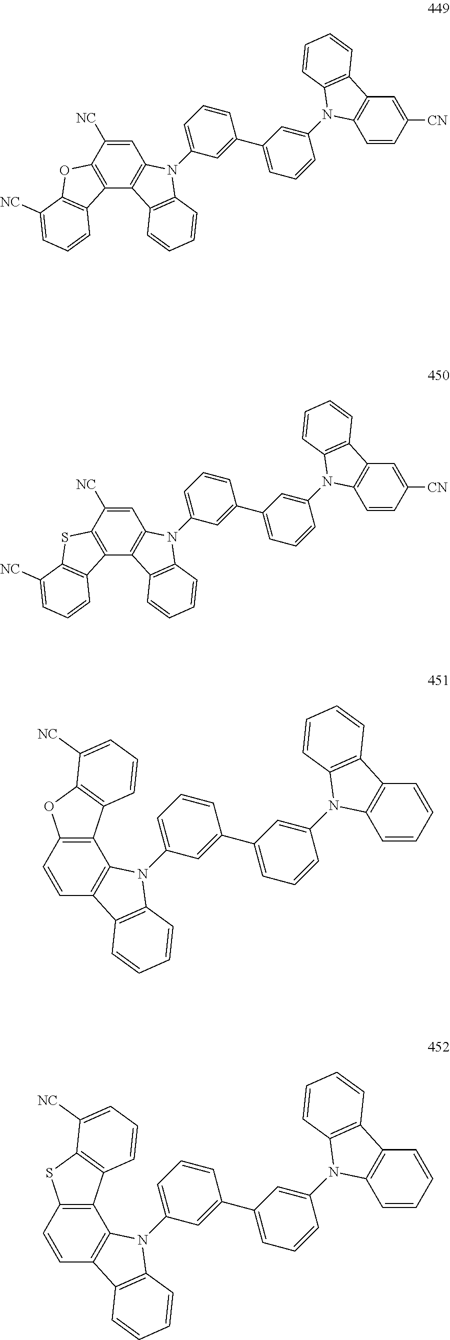 [SMILES: N#Cc1ccc2c(c1)c1ccccc1n2-c1cccc(-c2cccc(-n3c4ccccc4c4c5c(oc6c(C#N)cccc65)c(C#N)cc43)c2)c1 ; N#Cc1ccc2c(c1)c1ccccc1n2-c1cccc(-c2cccc(-n3c4ccccc4c4c5c(sc6c(C#N)cccc65)c(C#N)cc43)c2)c1 ; N#Cc1cccc2c1oc1ccc3c4ccccc4n(-c4cccc(-c5cccc(-n6c7ccccc7c7ccccc76)c5)c4)c3c12 ; N#Cc1cccc2c1sc1ccc3c4ccccc4n(-c4cccc(-c5cccc(-n6c7ccccc7c7ccccc76)c5)c4)c3c12]

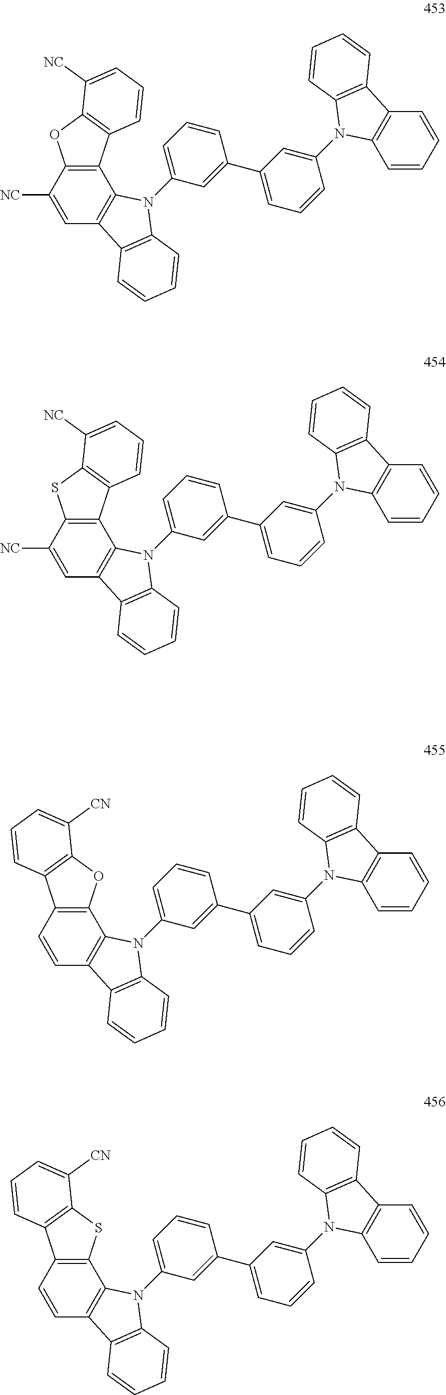 [SMILES: N#Cc1cccc2c1oc1c(C#N)cc3c4ccccc4n(-c4cccc(-c5cccc(-n6c7ccccc7c7ccccc76)c5)c4)c3c12 ; N#Cc1cccc2c1sc1c(C#N)cc3c4ccccc4n(-c4cccc(-c5cccc(-n6c7ccccc7c7ccccc76)c5)c4)c3c12 ; N#Cc1cccc2c1oc1c2ccc2c3ccccc3n(-c3cccc(-c4cccc(-n5c6ccccc6c6ccccc65)c4)c3)c21 ; N#Cc1cccc2c1sc1c2ccc2c3ccccc3n(-c3cccc(-c4cccc(-n5c6ccccc6c6ccccc65)c4)c3)c21]

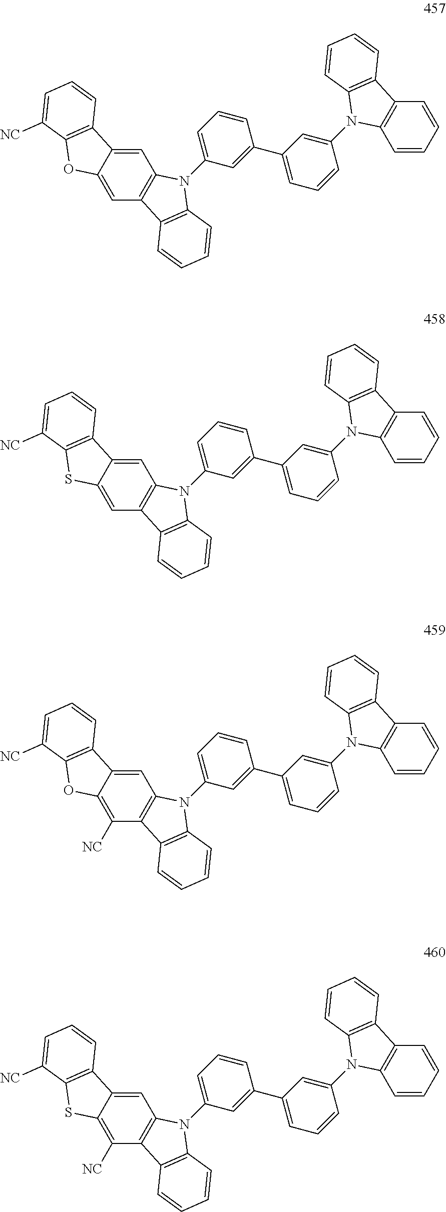 [SMILES: N#Cc1cccc2c1oc1cc3c4ccccc4n(-c4cccc(-c5cccc(-n6c7ccccc7c7ccccc76)c5)c4)c3cc12 ; N#Cc1cccc2c1sc1cc3c4ccccc4n(-c4cccc(-c5cccc(-n6c7ccccc7c7ccccc76)c5)c4)c3cc12 ; N#Cc1cccc2c1oc1c(C#N)c3c4ccccc4n(-c4cccc(-c5cccc(-n6c7ccccc7c7ccccc76)c5)c4)c3cc12 ; N#Cc1cccc2c1sc1c(C#N)c3c4ccccc4n(-c4cccc(-c5cccc(-n6c7ccccc7c7ccccc76)c5)c4)c3cc12]

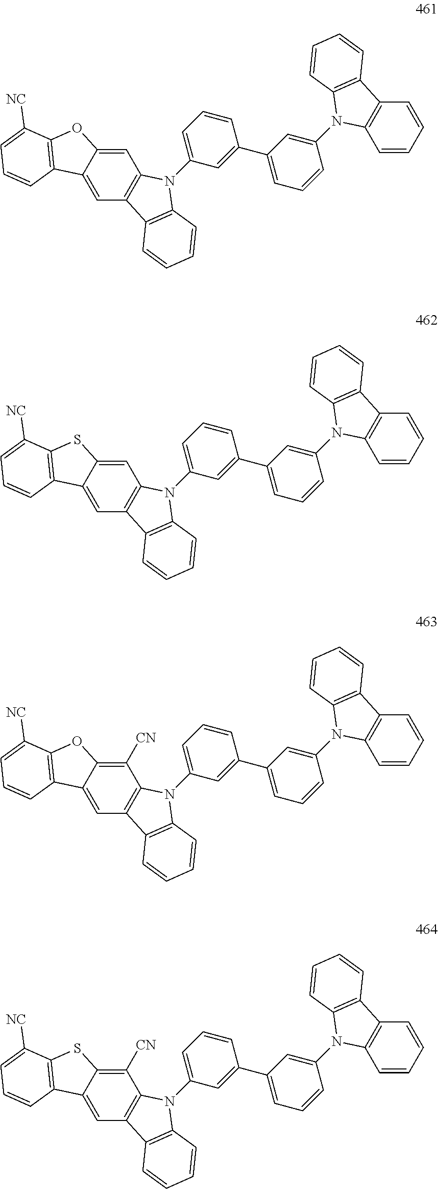 [SMILES: N#Cc1cccc2c1oc1cc3c(cc12)c1ccccc1n3-c1cccc(-c2cccc(-n3c4ccccc4c4ccccc43)c2)c1 ; N#Cc1cccc2c1sc1cc3c(cc12)c1ccccc1n3-c1cccc(-c2cccc(-n3c4ccccc4c4ccccc43)c2)c1 ; N#Cc1cccc2c1oc1c(C#N)c3c(cc12)c1ccccc1n3-c1cccc(-c2cccc(-n3c4ccccc4c4ccccc43)c2)c1 ; N#Cc1cccc2c1sc1c(C#N)c3c(cc12)c1ccccc1n3-c1cccc(-c2cccc(-n3c4ccccc4c4ccccc43)c2)c1]

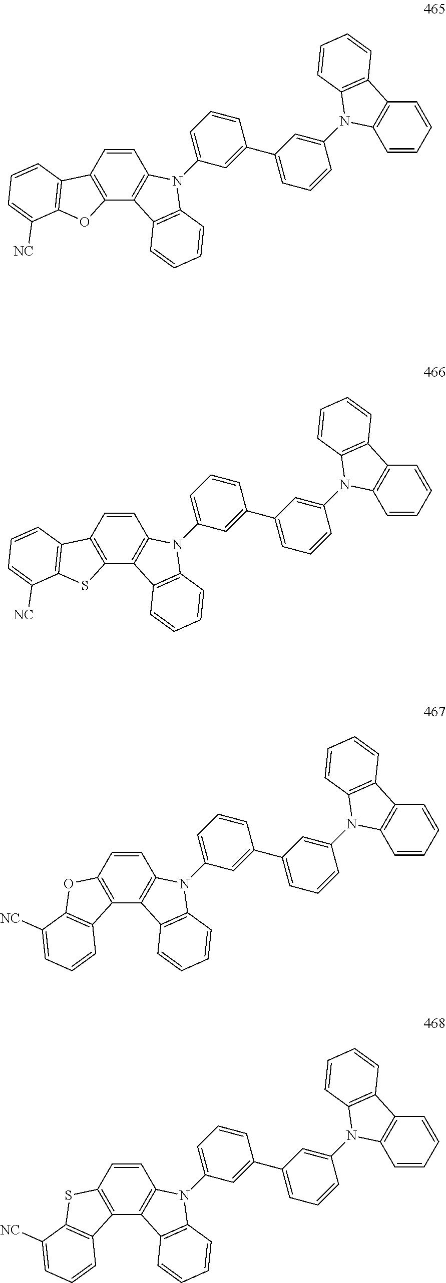 [SMILES: N#Cc1cccc2c1oc1c2ccc2c1c1ccccc1n2-c1cccc(-c2cccc(-n3c4ccccc4c4ccccc43)c2)c1 ; N#Cc1cccc2c1sc1c2ccc2c1c1ccccc1n2-c1cccc(-c2cccc(-n3c4ccccc4c4ccccc43)c2)c1 ; N#Cc1cccc2c1oc1ccc3c(c4ccccc4n3-c3cccc(-c4cccc(-n5c6ccccc6c6ccccc65)c4)c3)c12 ; N#Cc1cccc2c1sc1ccc3c(c4ccccc4n3-c3cccc(-c4cccc(-n5c6ccccc6c6ccccc65)c4)c3)c12]

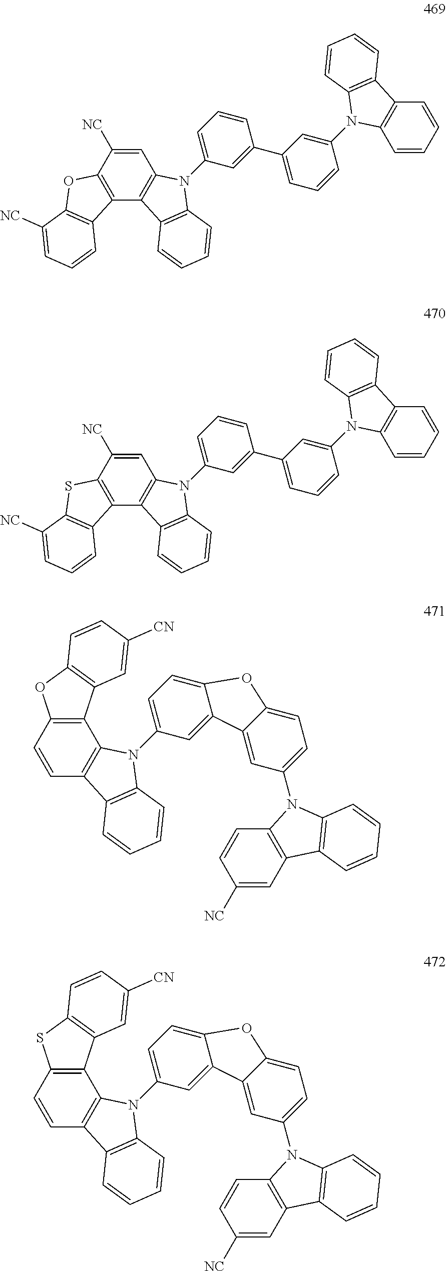 [SMILES: N#Cc1cccc2c1oc1c(C#N)cc3c(c4ccccc4n3-c3cccc(-c4cccc(-n5c6ccccc6c6ccccc65)c4)c3)c12 ; N#Cc1cccc2c1sc1c(C#N)cc3c(c4ccccc4n3-c3cccc(-c4cccc(-n5c6ccccc6c6ccccc65)c4)c3)c12 ; N#Cc1ccc2oc3ccc4c5ccccc5n(-c5ccc6oc7ccc(-n8c9ccccc9c9cc(C#N)ccc98)cc7c6c5)c4c3c2c1 ; N#Cc1ccc2sc3ccc4c5ccccc5n(-c5ccc6oc7ccc(-n8c9ccccc9c9cc(C#N)ccc98)cc7c6c5)c4c3c2c1]

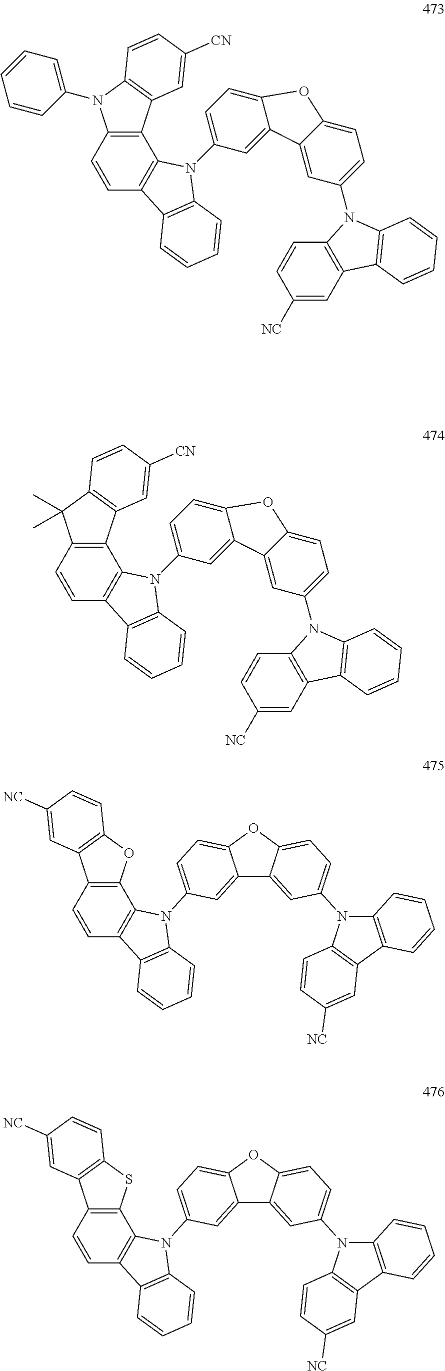 [SMILES: N#Cc1ccc2c(c1)c1ccccc1n2-c1ccc2oc3ccc(-n4c5ccccc5c5ccc6c(c7cc(C#N)ccc7n6-c6ccccc6)c54)cc3c2c1 ; CC1(C)c2ccc(C#N)cc2-c2c1ccc1c3ccccc3n(-c3ccc4oc5ccc(-n6c7ccccc7c7cc(C#N)ccc76)cc5c4c3)c21 ; N#Cc1ccc2oc3c(ccc4c5ccccc5n(-c5ccc6oc7ccc(-n8c9ccccc9c9cc(C#N)ccc98)cc7c6c5)c43)c2c1 ; N#Cc1ccc2sc3c(ccc4c5ccccc5n(-c5ccc6oc7ccc(-n8c9ccccc9c9cc(C#N)ccc98)cc7c6c5)c43)c2c1]

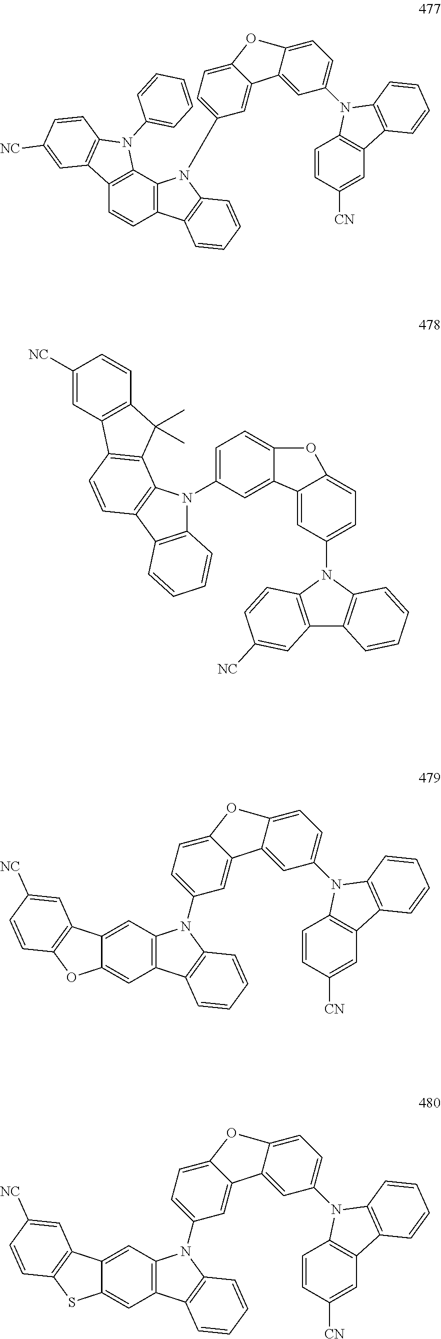 [SMILES: N#Cc1ccc2c(c1)c1ccccc1n2-c1ccc2oc3ccc(-n4c5ccccc5c5ccc6c7cc(C#N)ccc7n(-c7ccccc7)c6c54)cc3c2c1 ; CC1(C)c2ccc(C#N)cc2-c2ccc3c4ccccc4n(-c4ccc5oc6ccc(-n7c8ccccc8c8cc(C#N)ccc87)cc6c5c4)c3c21 ; N#Cc1ccc2oc3cc4c5ccccc5n(-c5ccc6oc7ccc(-n8c9ccccc9c9cc(C#N)ccc98)cc7c6c5)c4cc3c2c1 ; N#Cc1ccc2sc3cc4c5ccccc5n(-c5ccc6oc7ccc(-n8c9ccccc9c9cc(C#N)ccc98)cc7c6c5)c4cc3c2c1]

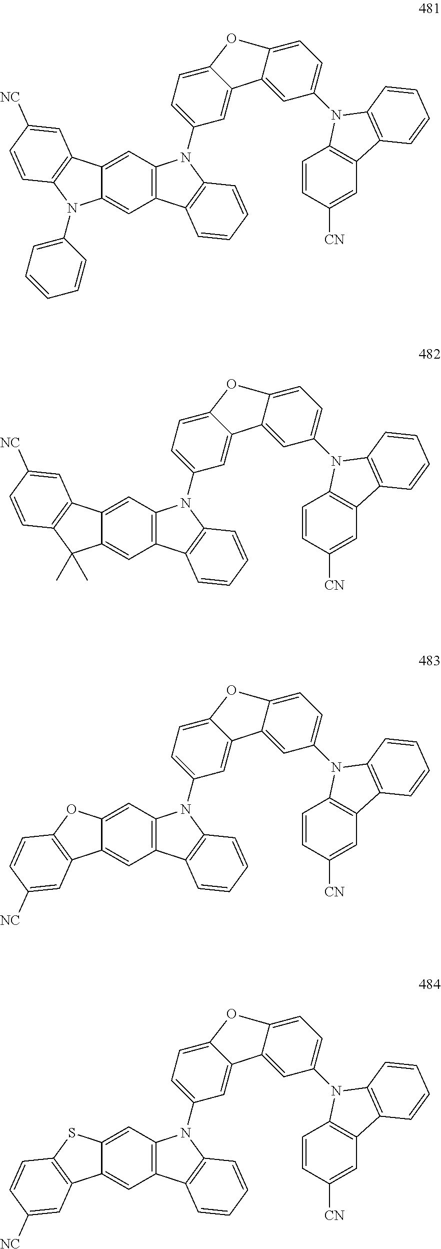 [SMILES: N#Cc1ccc2c(c1)c1ccccc1n2-c1ccc2oc3ccc(-n4c5ccccc5c5cc6c(cc54)c4cc(C#N)ccc4n6-c4ccccc4)cc3c2c1 ; CC1(C)c2ccc(C#N)cc2-c2cc3c(cc21)c1ccccc1n3-c1ccc2oc3ccc(-n4c5ccccc5c5cc(C#N)ccc54)cc3c2c1 ; N#Cc1ccc2oc3cc4c(cc3c2c1)c1ccccc1n4-c1ccc2oc3ccc(-n4c5ccccc5c5cc(C#N)ccc54)cc3c2c1 ; N#Cc1ccc2sc3cc4c(cc3c2c1)c1ccccc1n4-c1ccc2oc3ccc(-n4c5ccccc5c5cc(C#N)ccc54)cc3c2c1]

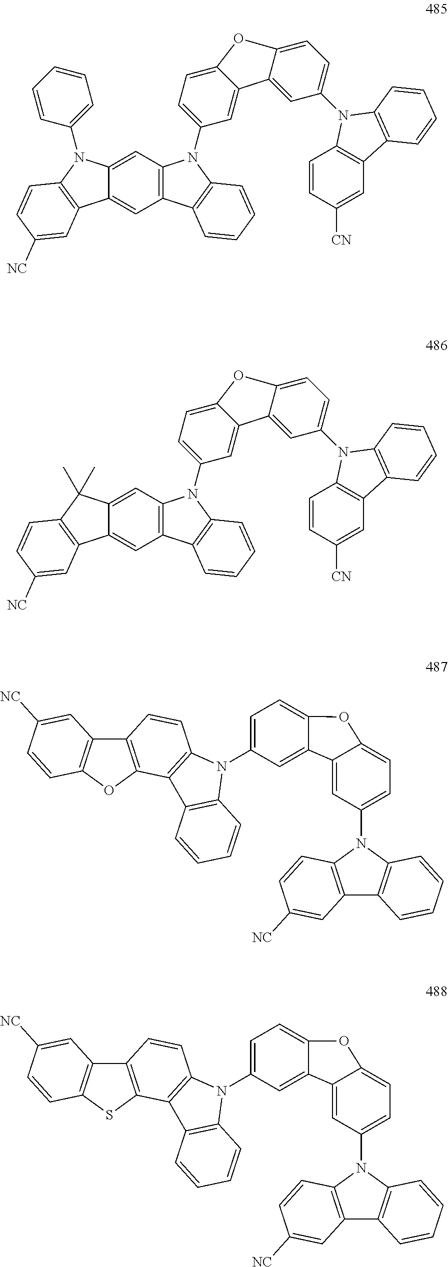 [SMILES: N#Cc1ccc2c(c1)c1ccccc1n2-c1ccc2oc3ccc(-n4c5ccccc5c5cc6c7cc(C#N)ccc7n(-c7ccccc7)c6cc54)cc3c2c1 ; CC1(C)c2ccc(C#N)cc2-c2cc3c4ccccc4n(-c4ccc5oc6ccc(-n7c8ccccc8c8cc(C#N)ccc87)cc6c5c4)c3cc21 ; N#Cc1ccc2oc3c(ccc4c3c3ccccc3n4-c3ccc4oc5ccc(-n6c7ccccc7c7cc(C#N)ccc76)cc5c4c3)c2c1 ; N#Cc1ccc2sc3c(ccc4c3c3ccccc3n4-c3ccc4oc5ccc(-n6c7ccccc7c7cc(C#N)ccc76)cc5c4c3)c2c1]

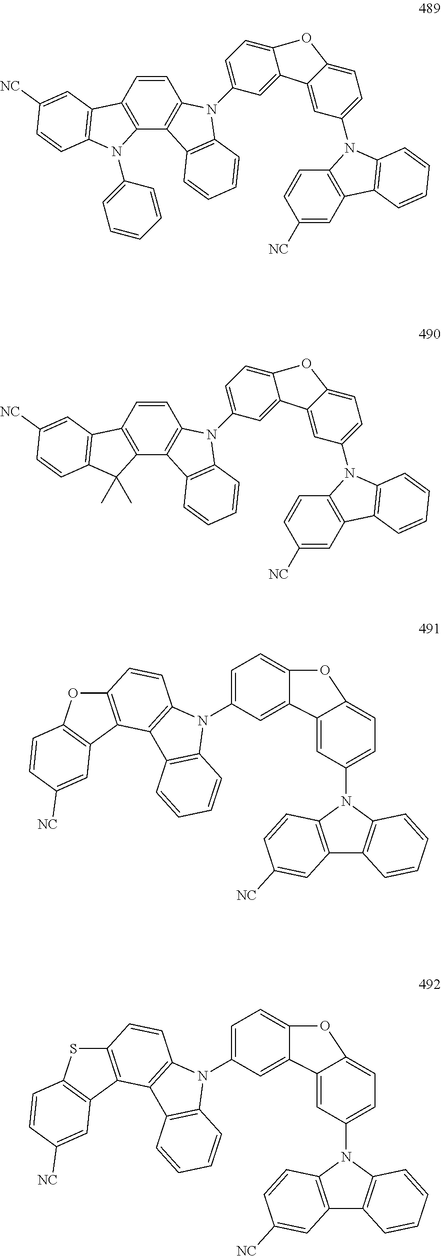 [SMILES: N#Cc1ccc2c(c1)c1ccccc1n2-c1ccc2oc3ccc(-n4c5ccccc5c5c4ccc4c6cc(C#N)ccc6n(-c6ccccc6)c45)cc3c2c1 ; CC1(C)c2ccc(C#N)cc2-c2ccc3c(c21)c1ccccc1n3-c1ccc2oc3ccc(-n4c5ccccc5c5cc(C#N)ccc54)cc3c2c1 ; N#Cc1ccc2oc3ccc4c(c5ccccc5n4-c4ccc5oc6ccc(-n7c8ccccc8c8cc(C#N)ccc87)cc6c5c4)c3c2c1 ; N#Cc1ccc2sc3ccc4c(c5ccccc5n4-c4ccc5oc6ccc(-n7c8ccccc8c8cc(C#N)ccc87)cc6c5c4)c3c2c1]

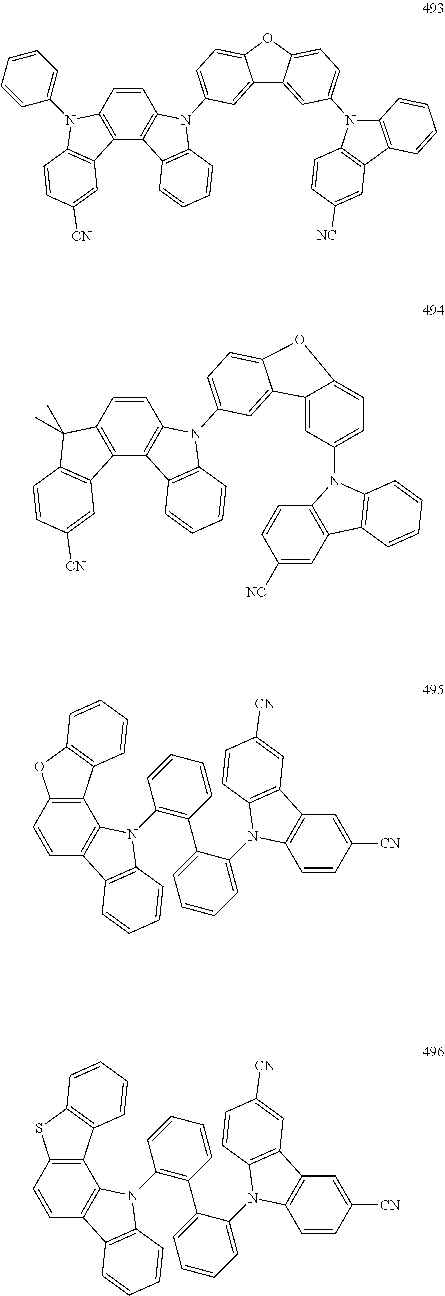 [SMILES: N#Cc1ccc2c(c1)c1ccccc1n2-c1ccc2oc3ccc(-n4c5ccccc5c5c6c7cc(C#N)ccc7n(-c7ccccc7)c6ccc54)cc3c2c1 ; CC1(C)c2ccc(C#N)cc2-c2c1ccc1c2c2ccccc2n1-c1ccc2oc3ccc(-n4c5ccccc5c5cc(C#N)ccc54)cc3c2c1 ; N#Cc1ccc2c(c1)c1cc(C#N)ccc1n2-c1ccccc1-c1ccccc1-n1c2ccccc2c2ccc3oc4ccccc4c3c21 ; N#Cc1ccc2c(c1)c1cc(C#N)ccc1n2-c1ccccc1-c1ccccc1-n1c2ccccc2c2ccc3sc4ccccc4c3c21]

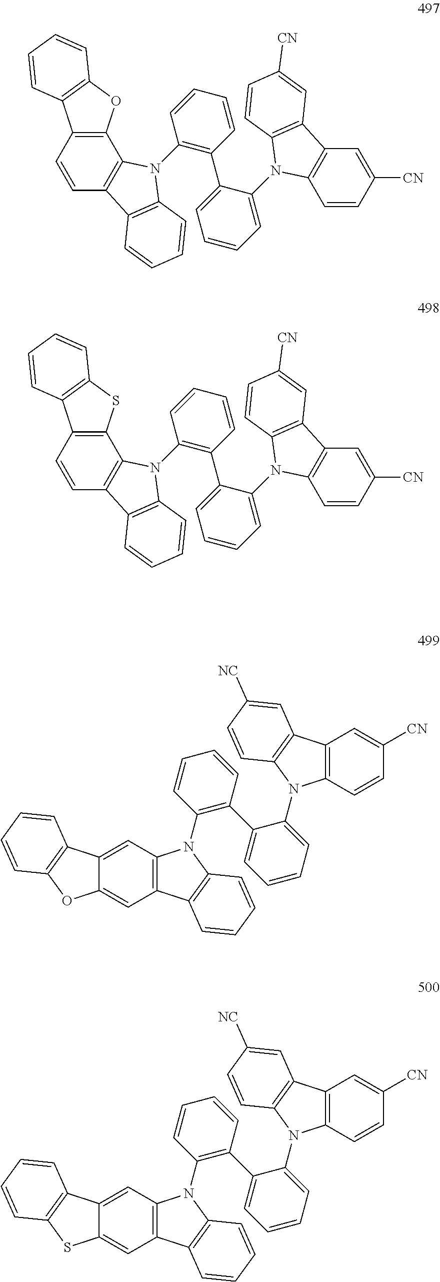 [SMILES: N#Cc1ccc2c(c1)c1cc(C#N)ccc1n2-c1ccccc1-c1ccccc1-n1c2ccccc2c2ccc3c4ccccc4oc3c21 ; N#Cc1ccc2c(c1)c1cc(C#N)ccc1n2-c1ccccc1-c1ccccc1-n1c2ccccc2c2ccc3c4ccccc4sc3c21 ; N#Cc1ccc2c(c1)c1cc(C#N)ccc1n2-c1ccccc1-c1ccccc1-n1c2ccccc2c2cc3oc4ccccc4c3cc21 ; N#Cc1ccc2c(c1)c1cc(C#N)ccc1n2-c1ccccc1-c1ccccc1-n1c2ccccc2c2cc3sc4ccccc4c3cc21]

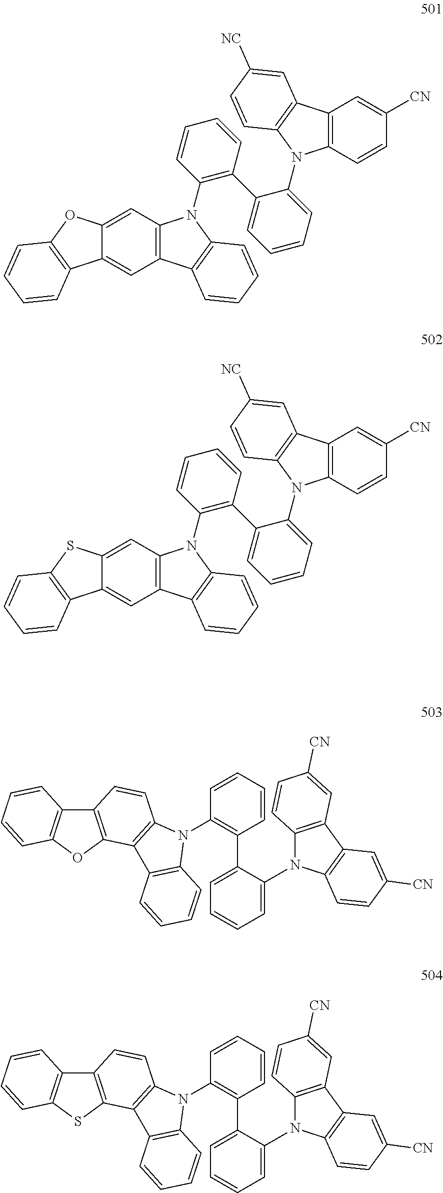 [SMILES: N#Cc1ccc2c(c1)c1cc(C#N)ccc1n2-c1ccccc1-c1ccccc1-n1c2ccccc2c2cc3c(cc21)oc1ccccc13 ; N#Cc1ccc2c(c1)c1cc(C#N)ccc1n2-c1ccccc1-c1ccccc1-n1c2ccccc2c2cc3c(cc21)sc1ccccc13 ; N#Cc1ccc2c(c1)c1cc(C#N)ccc1n2-c1ccccc1-c1ccccc1-n1c2ccccc2c2c3oc4ccccc4c3ccc21 ; N#Cc1ccc2c(c1)c1cc(C#N)ccc1n2-c1ccccc1-c1ccccc1-n1c2ccccc2c2c3sc4ccccc4c3ccc21]

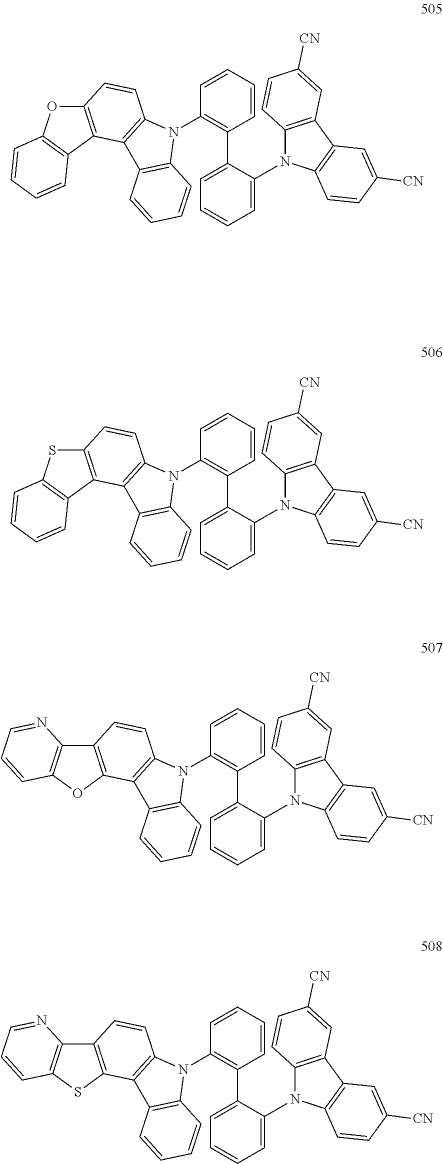 [SMILES: N#Cc1ccc2c(c1)c1cc(C#N)ccc1n2-c1ccccc1-c1ccccc1-n1c2ccccc2c2c3c(ccc21)oc1ccccc13 ; N#Cc1ccc2c(c1)c1cc(C#N)ccc1n2-c1ccccc1-c1ccccc1-n1c2ccccc2c2c3c(ccc21)sc1ccccc13 ; N#Cc1ccc2c(c1)c1cc(C#N)ccc1n2-c1ccccc1-c1ccccc1-n1c2ccccc2c2c3oc4cccnc4c3ccc21 ; N#Cc1ccc2c(c1)c1cc(C#N)ccc1n2-c1ccccc1-c1ccccc1-n1c2ccccc2c2c3sc4cccnc4c3ccc21]

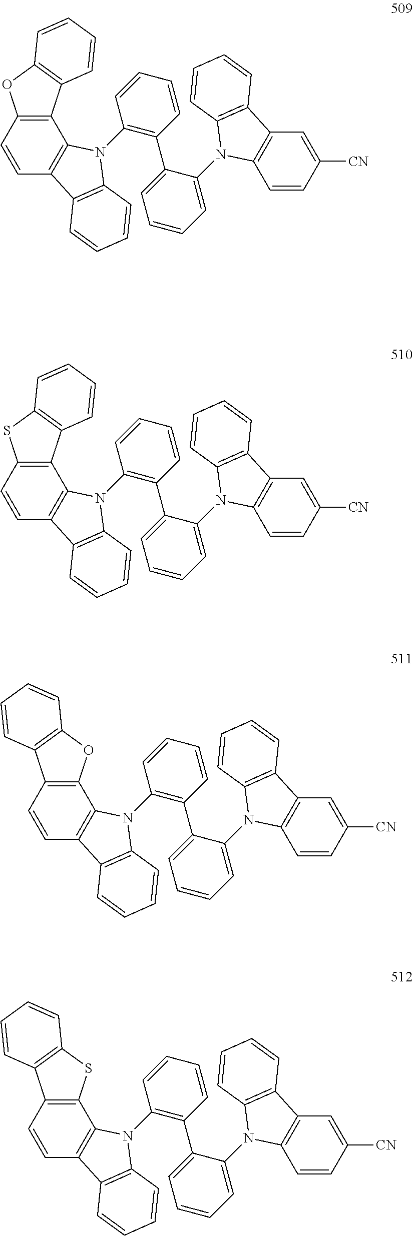 [SMILES: N#Cc1ccc2c(c1)c1ccccc1n2-c1ccccc1-c1ccccc1-n1c2ccccc2c2ccc3oc4ccccc4c3c21 ; N#Cc1ccc2c(c1)c1ccccc1n2-c1ccccc1-c1ccccc1-n1c2ccccc2c2ccc3sc4ccccc4c3c21 ; N#Cc1ccc2c(c1)c1ccccc1n2-c1ccccc1-c1ccccc1-n1c2ccccc2c2ccc3c4ccccc4oc3c21 ; N#Cc1ccc2c(c1)c1ccccc1n2-c1ccccc1-c1ccccc1-n1c2ccccc2c2ccc3c4ccccc4sc3c21]

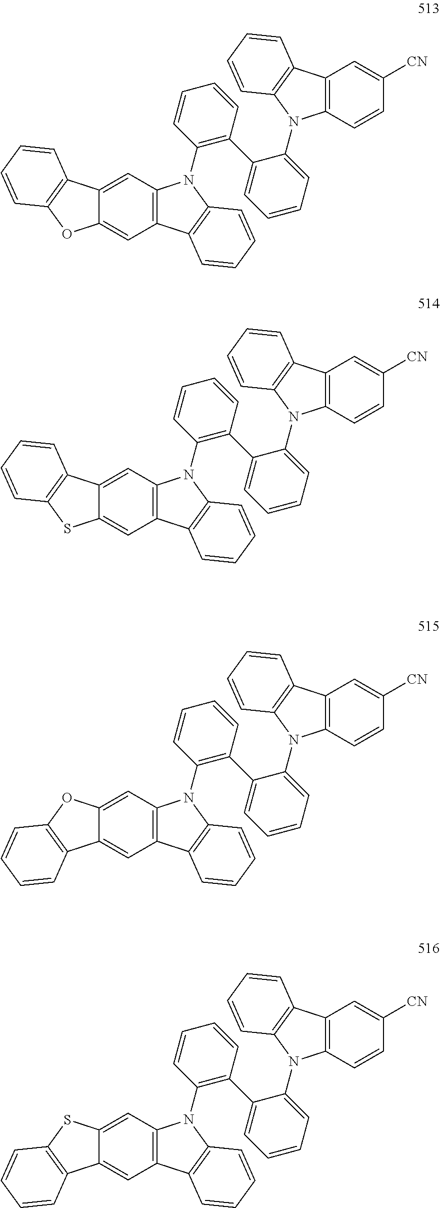 [SMILES: N#Cc1ccc2c(c1)c1ccccc1n2-c1ccccc1-c1ccccc1-n1c2ccccc2c2cc3oc4ccccc4c3cc21 ; N#Cc1ccc2c(c1)c1ccccc1n2-c1ccccc1-c1ccccc1-n1c2ccccc2c2cc3sc4ccccc4c3cc21 ; N#Cc1ccc2c(c1)c1ccccc1n2-c1ccccc1-c1ccccc1-n1c2ccccc2c2cc3c(cc21)oc1ccccc13 ; N#Cc1ccc2c(c1)c1ccccc1n2-c1ccccc1-c1ccccc1-n1c2ccccc2c2cc3c(cc21)sc1ccccc13]

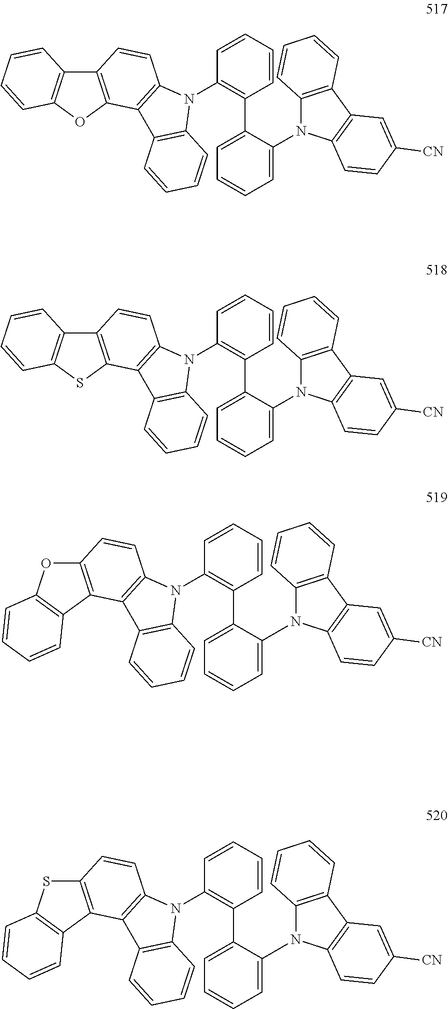 [SMILES: N#Cc1ccc2c(c1)c1ccccc1n2-c1ccccc1-c1ccccc1-n1c2ccccc2c2c3oc4ccccc4c3ccc21 ; N#Cc1ccc2c(c1)c1ccccc1n2-c1ccccc1-c1ccccc1-n1c2ccccc2c2c3sc4ccccc4c3ccc21 ; N#Cc1ccc2c(c1)c1ccccc1n2-c1ccccc1-c1ccccc1-n1c2ccccc2c2c3c(ccc21)oc1ccccc13 ; N#Cc1ccc2c(c1)c1ccccc1n2-c1ccccc1-c1ccccc1-n1c2ccccc2c2c3c(ccc21)sc1ccccc13]

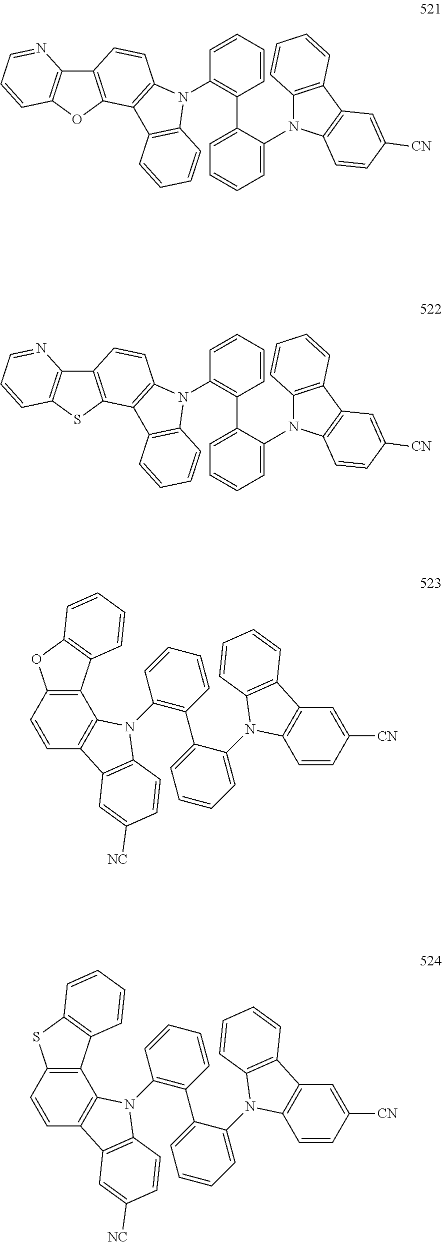 [SMILES: N#Cc1ccc2c(c1)c1ccccc1n2-c1ccccc1-c1ccccc1-n1c2ccccc2c2c3oc4cccnc4c3ccc21 ; N#Cc1ccc2c(c1)c1ccccc1n2-c1ccccc1-c1ccccc1-n1c2ccccc2c2c3sc4cccnc4c3ccc21 ; N#Cc1ccc2c(c1)c1ccccc1n2-c1ccccc1-c1ccccc1-n1c2ccc(C#N)cc2c2ccc3oc4ccccc4c3c21 ; N#Cc1ccc2c(c1)c1ccccc1n2-c1ccccc1-c1ccccc1-n1c2ccc(C#N)cc2c2ccc3sc4ccccc4c3c21]

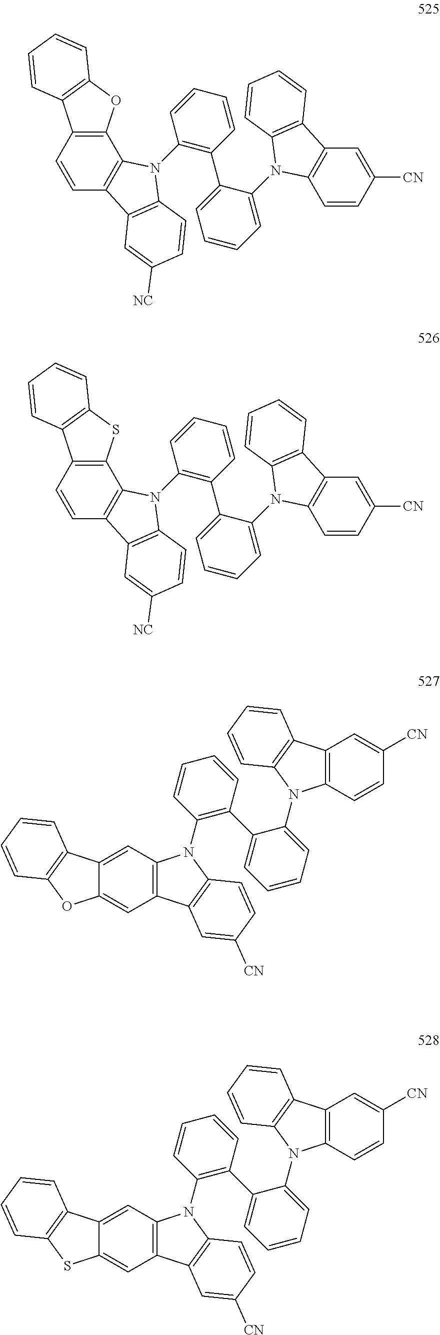 [SMILES: N#Cc1ccc2c(c1)c1ccccc1n2-c1ccccc1-c1ccccc1-n1c2ccc(C#N)cc2c2ccc3c4ccccc4oc3c21 ; N#Cc1ccc2c(c1)c1ccccc1n2-c1ccccc1-c1ccccc1-n1c2ccc(C#N)cc2c2ccc3c4ccccc4sc3c21 ; N#Cc1ccc2c(c1)c1ccccc1n2-c1ccccc1-c1ccccc1-n1c2ccc(C#N)cc2c2cc3oc4ccccc4c3cc21 ; N#Cc1ccc2c(c1)c1ccccc1n2-c1ccccc1-c1ccccc1-n1c2ccc(C#N)cc2c2cc3sc4ccccc4c3cc21]

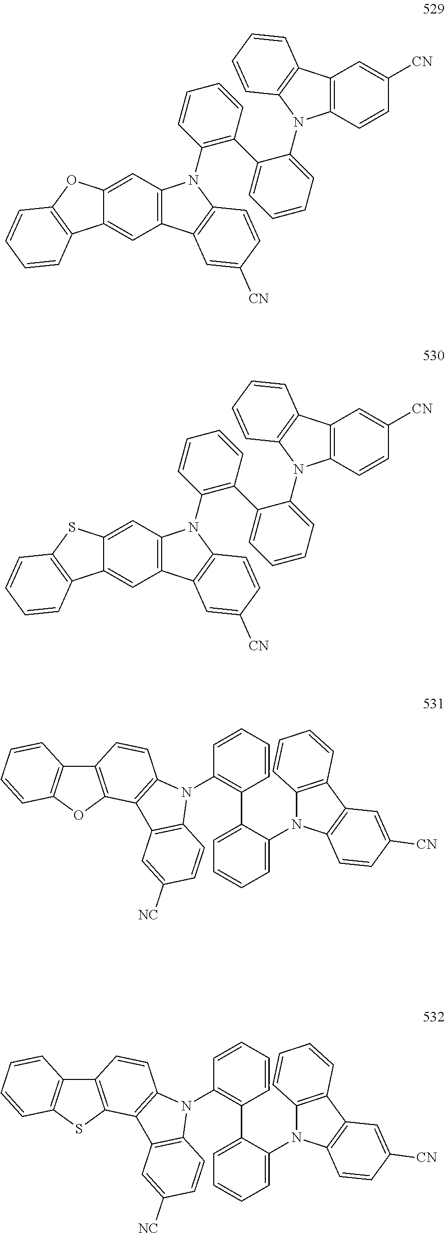 [SMILES: N#Cc1ccc2c(c1)c1ccccc1n2-c1ccccc1-c1ccccc1-n1c2ccc(C#N)cc2c2cc3c(cc21)oc1ccccc13 ; N#Cc1ccc2c(c1)c1ccccc1n2-c1ccccc1-c1ccccc1-n1c2ccc(C#N)cc2c2cc3c(cc21)sc1ccccc13 ; N#Cc1ccc2c(c1)c1ccccc1n2-c1ccccc1-c1ccccc1-n1c2ccc(C#N)cc2c2c3oc4ccccc4c3ccc21 ; N#Cc1ccc2c(c1)c1ccccc1n2-c1ccccc1-c1ccccc1-n1c2ccc(C#N)cc2c2c3sc4ccccc4c3ccc21]

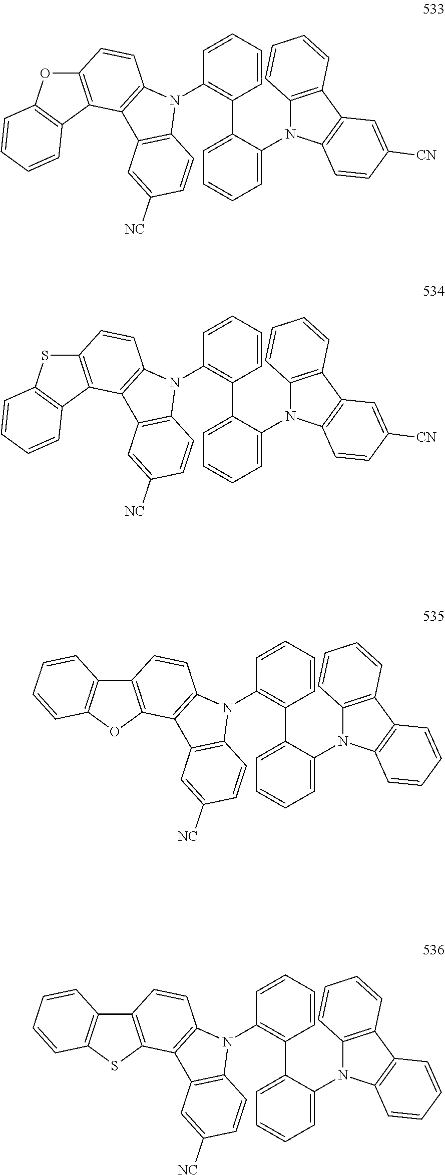 [SMILES: N#Cc1ccc2c(c1)c1ccccc1n2-c1ccccc1-c1ccccc1-n1c2ccc(C#N)cc2c2c3c(ccc21)oc1ccccc13 ; N#Cc1ccc2c(c1)c1ccccc1n2-c1ccccc1-c1ccccc1-n1c2ccc(C#N)cc2c2c3c(ccc21)sc1ccccc13 ; N#Cc1ccc2c(c1)c1c3oc4ccccc4c3ccc1n2-c1ccccc1-c1ccccc1-n1c2ccccc2c2ccccc21 ; N#Cc1ccc2c(c1)c1c3sc4ccccc4c3ccc1n2-c1ccccc1-c1ccccc1-n1c2ccccc2c2ccccc21]

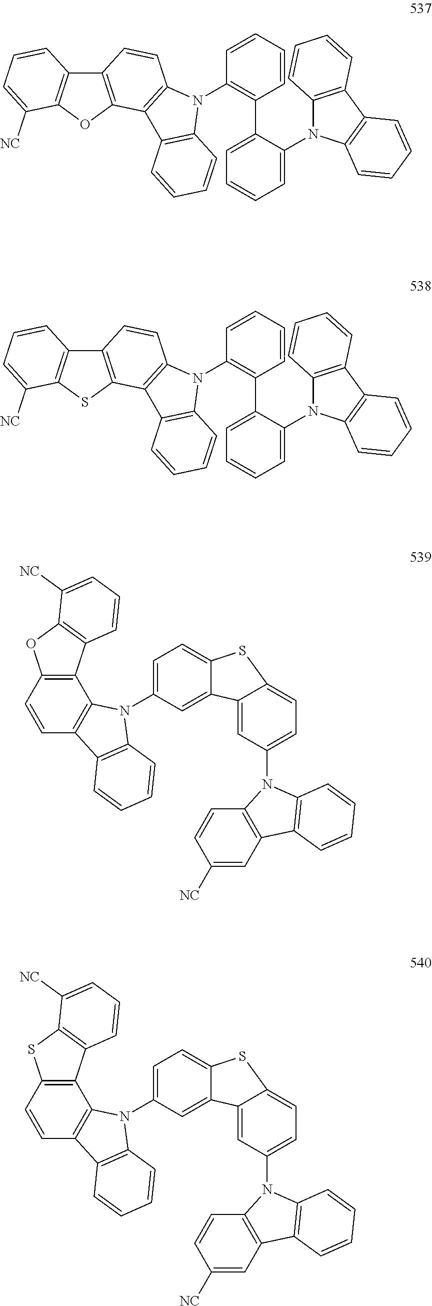 [SMILES: N#Cc1cccc2c1oc1c2ccc2c1c1ccccc1n2-c1ccccc1-c1ccccc1-n1c2ccccc2c2ccccc21 ; N#Cc1cccc2c1sc1c2ccc2c1c1ccccc1n2-c1ccccc1-c1ccccc1-n1c2ccccc2c2ccccc21 ; N#Cc1ccc2c(c1)c1ccccc1n2-c1ccc2sc3ccc(-n4c5ccccc5c5ccc6oc7c(C#N)cccc7c6c54)cc3c2c1 ; N#Cc1ccc2c(c1)c1ccccc1n2-c1ccc2sc3ccc(-n4c5ccccc5c5ccc6sc7c(C#N)cccc7c6c54)cc3c2c1]

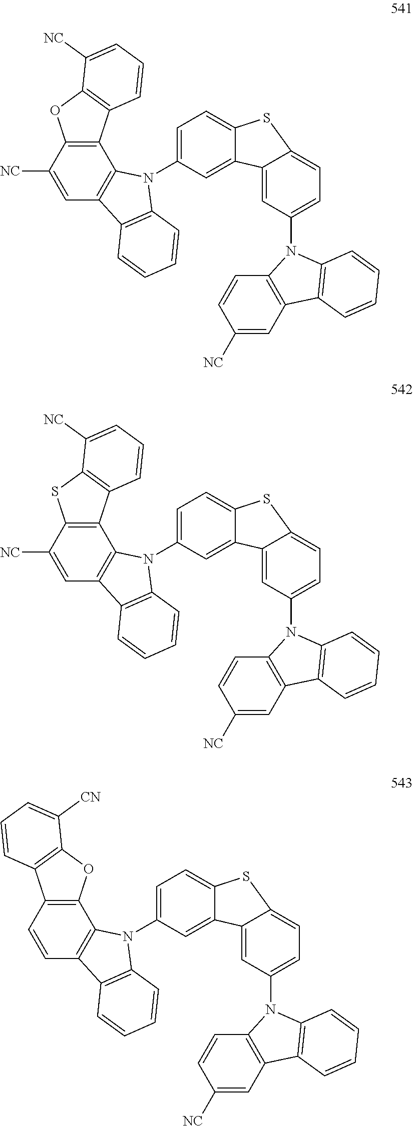 [SMILES: N#Cc1ccc2c(c1)c1ccccc1n2-c1ccc2sc3ccc(-n4c5ccccc5c5cc(C#N)c6oc7c(C#N)cccc7c6c54)cc3c2c1 ; N#Cc1ccc2c(c1)c1ccccc1n2-c1ccc2sc3ccc(-n4c5ccccc5c5cc(C#N)c6sc7c(C#N)cccc7c6c54)cc3c2c1 ; N#Cc1ccc2c(c1)c1ccccc1n2-c1ccc2sc3ccc(-n4c5ccccc5c5ccc6c7cccc(C#N)c7oc6c54)cc3c2c1]

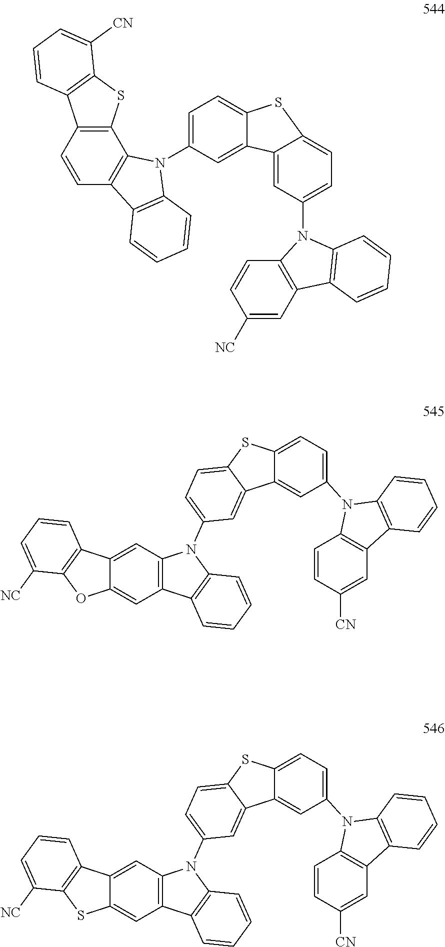 [SMILES: N#Cc1ccc2c(c1)c1ccccc1n2-c1ccc2sc3ccc(-n4c5ccccc5c5ccc6c7cccc(C#N)c7sc6c54)cc3c2c1 ; N#Cc1ccc2c(c1)c1ccccc1n2-c1ccc2sc3ccc(-n4c5ccccc5c5cc6oc7c(C#N)cccc7c6cc54)cc3c2c1 ; N#Cc1ccc2c(c1)c1ccccc1n2-c1ccc2sc3ccc(-n4c5ccccc5c5cc6sc7c(C#N)cccc7c6cc54)cc3c2c1]

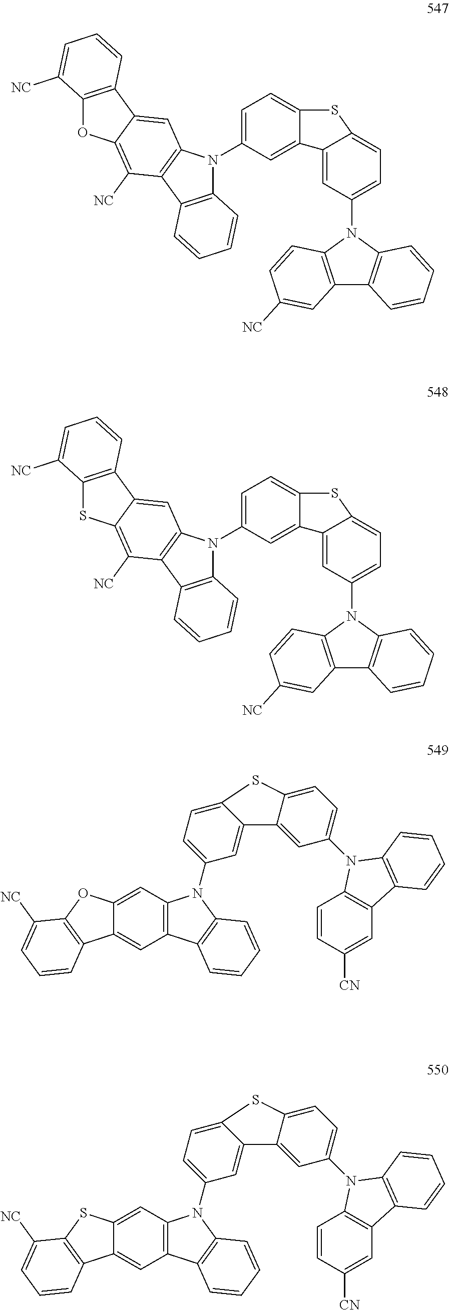 [SMILES: N#Cc1ccc2c(c1)c1ccccc1n2-c1ccc2sc3ccc(-n4c5ccccc5c5c(C#N)c6oc7c(C#N)cccc7c6cc54)cc3c2c1 ; N#Cc1ccc2c(c1)c1ccccc1n2-c1ccc2sc3ccc(-n4c5ccccc5c5c(C#N)c6sc7c(C#N)cccc7c6cc54)cc3c2c1 ; N#Cc1ccc2c(c1)c1ccccc1n2-c1ccc2sc3ccc(-n4c5ccccc5c5cc6c(cc54)oc4c(C#N)cccc46)cc3c2c1 ; N#Cc1ccc2c(c1)c1ccccc1n2-c1ccc2sc3ccc(-n4c5ccccc5c5cc6c(cc54)sc4c(C#N)cccc46)cc3c2c1]

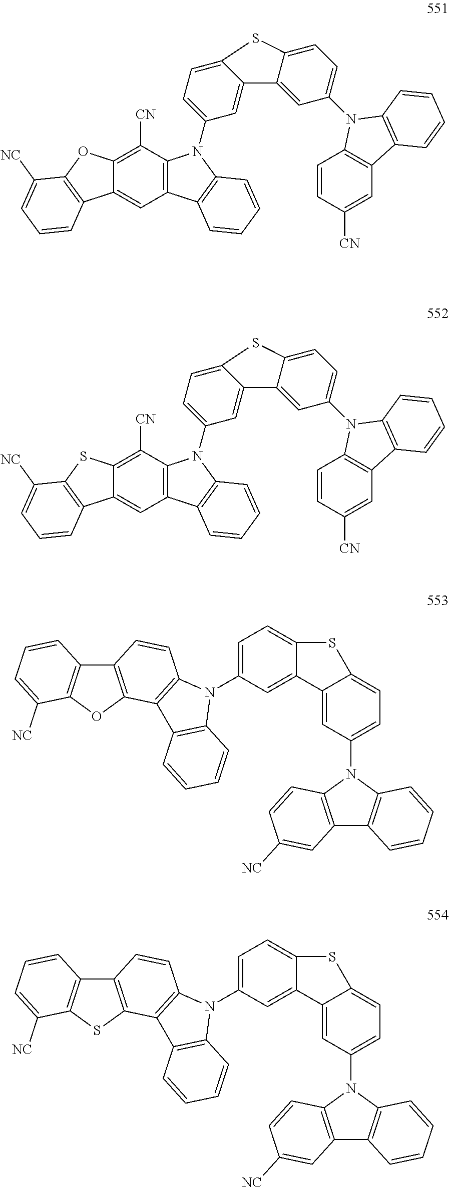 [SMILES: N#Cc1ccc2c(c1)c1ccccc1n2-c1ccc2sc3ccc(-n4c5ccccc5c5cc6c(oc7c(C#N)cccc76)c(C#N)c54)cc3c2c1 ; N#Cc1ccc2c(c1)c1ccccc1n2-c1ccc2sc3ccc(-n4c5ccccc5c5cc6c(sc7c(C#N)cccc76)c(C#N)c54)cc3c2c1 ; N#Cc1ccc2c(c1)c1ccccc1n2-c1ccc2sc3ccc(-n4c5ccccc5c5c6oc7c(C#N)cccc7c6ccc54)cc3c2c1 ; N#Cc1ccc2c(c1)c1ccccc1n2-c1ccc2sc3ccc(-n4c5ccccc5c5c6sc7c(C#N)cccc7c6ccc54)cc3c2c1]

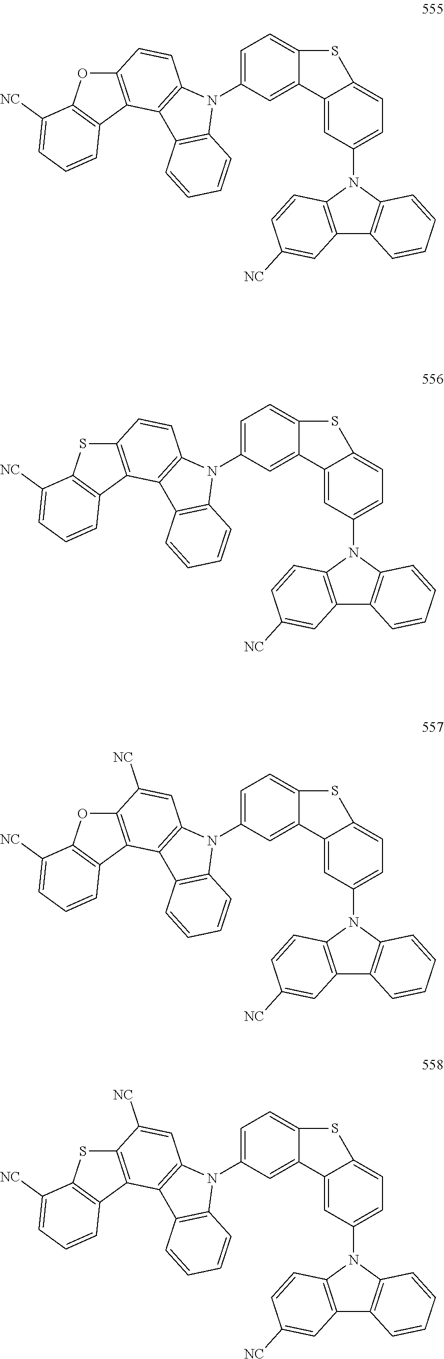 [SMILES: N#Cc1ccc2c(c1)c1ccccc1n2-c1ccc2sc3ccc(-n4c5ccccc5c5c6c(ccc54)oc4c(C#N)cccc46)cc3c2c1 ; N#Cc1ccc2c(c1)c1ccccc1n2-c1ccc2sc3ccc(-n4c5ccccc5c5c6c(ccc54)sc4c(C#N)cccc46)cc3c2c1 ; N#Cc1ccc2c(c1)c1ccccc1n2-c1ccc2sc3ccc(-n4c5ccccc5c5c6c(oc7c(C#N)cccc76)c(C#N)cc54)cc3c2c1 ; N#Cc1ccc2c(c1)c1ccccc1n2-c1ccc2sc3ccc(-n4c5ccccc5c5c6c(sc7c(C#N)cccc76)c(C#N)cc54)cc3c2c1]

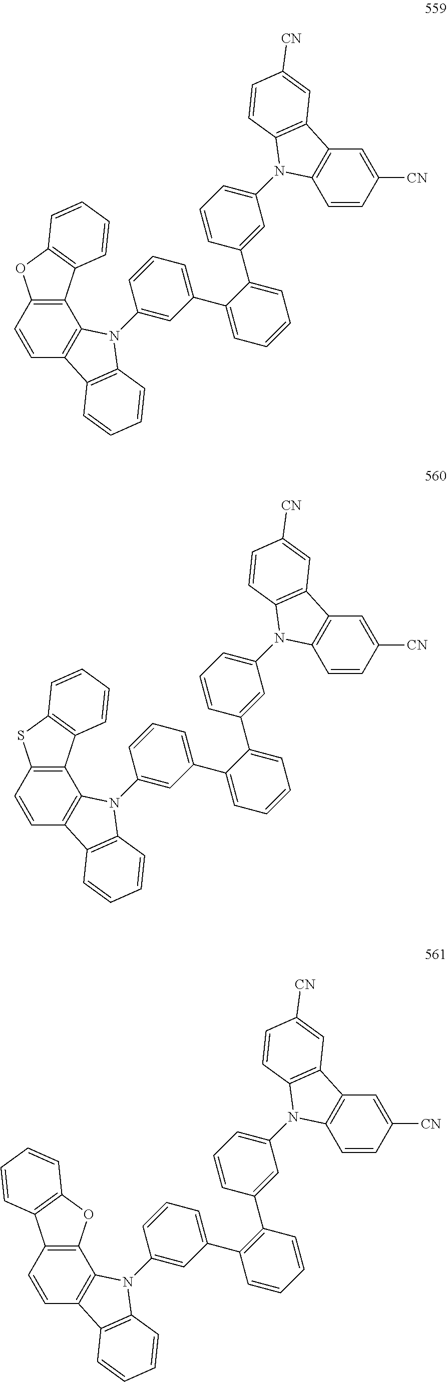 [SMILES: N#Cc1ccc2c(c1)c1cc(C#N)ccc1n2-c1cccc(-c2ccccc2-c2cccc(-n3c4ccccc4c4ccc5oc6ccccc6c5c43)c2)c1 ; N#Cc1ccc2c(c1)c1cc(C#N)ccc1n2-c1cccc(-c2ccccc2-c2cccc(-n3c4ccccc4c4ccc5sc6ccccc6c5c43)c2)c1 ; N#Cc1ccc2c(c1)c1cc(C#N)ccc1n2-c1cccc(-c2ccccc2-c2cccc(-n3c4ccccc4c4ccc5c6ccccc6oc5c43)c2)c1]

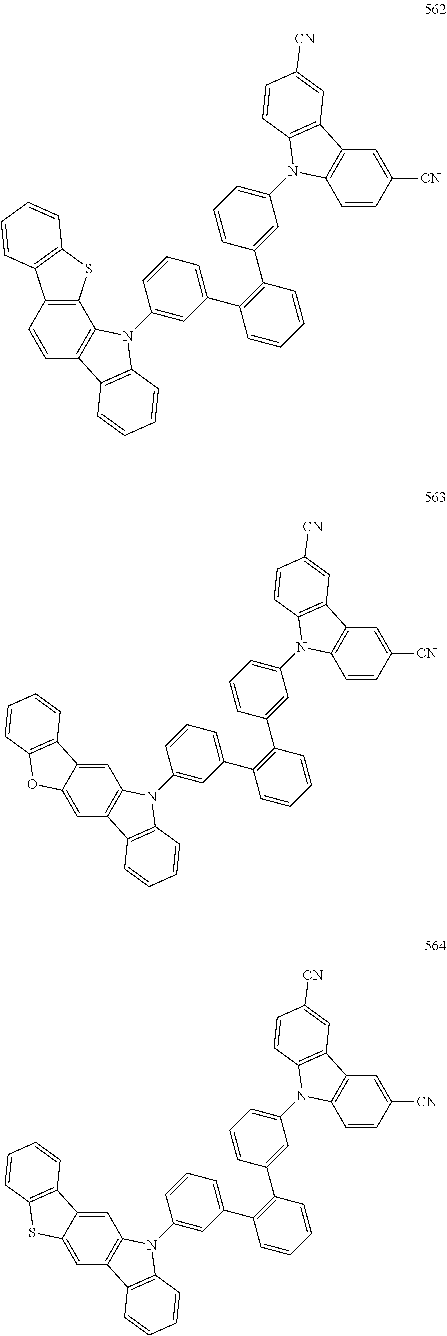 [SMILES: N#Cc1ccc2c(c1)c1cc(C#N)ccc1n2-c1cccc(-c2ccccc2-c2cccc(-n3c4ccccc4c4ccc5c6ccccc6sc5c43)c2)c1 ; N#Cc1ccc2c(c1)c1cc(C#N)ccc1n2-c1cccc(-c2ccccc2-c2cccc(-n3c4ccccc4c4cc5oc6ccccc6c5cc43)c2)c1 ; N#Cc1ccc2c(c1)c1cc(C#N)ccc1n2-c1cccc(-c2ccccc2-c2cccc(-n3c4ccccc4c4cc5sc6ccccc6c5cc43)c2)c1]

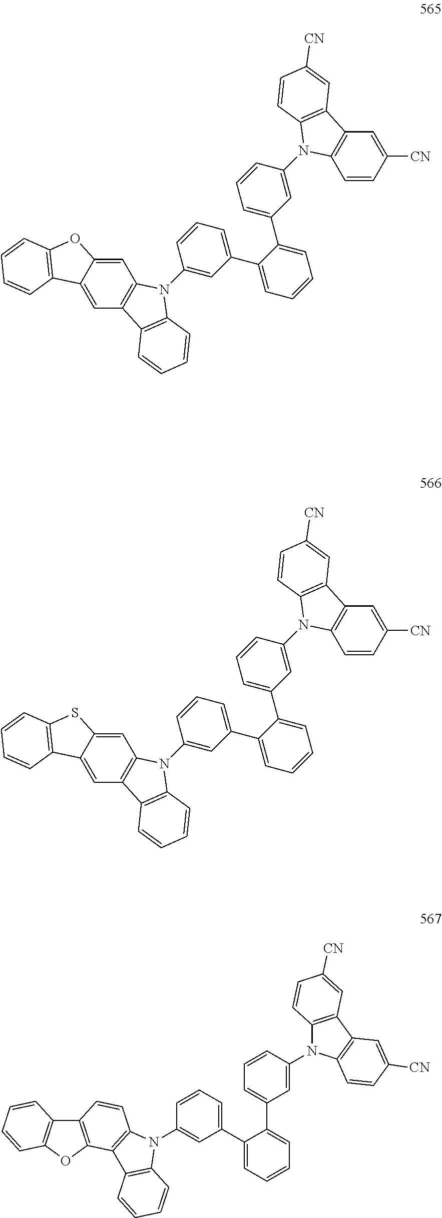 [SMILES: N#Cc1ccc2c(c1)c1cc(C#N)ccc1n2-c1cccc(-c2ccccc2-c2cccc(-n3c4ccccc4c4cc5c(cc43)oc3ccccc35)c2)c1 ; N#Cc1ccc2c(c1)c1cc(C#N)ccc1n2-c1cccc(-c2ccccc2-c2cccc(-n3c4ccccc4c4cc5c(cc43)sc3ccccc35)c2)c1 ; N#Cc1ccc2c(c1)c1cc(C#N)ccc1n2-c1cccc(-c2ccccc2-c2cccc(-n3c4ccccc4c4c5oc6ccccc6c5ccc43)c2)c1]

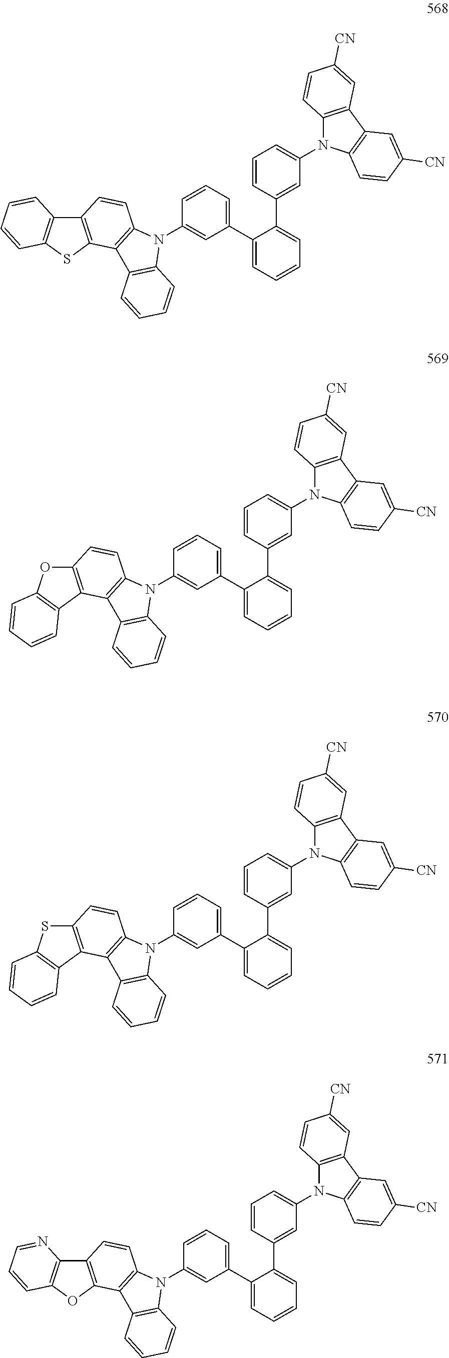 [SMILES: N#Cc1ccc2c(c1)c1cc(C#N)ccc1n2-c1cccc(-c2ccccc2-c2cccc(-n3c4ccccc4c4c5sc6ccccc6c5ccc43)c2)c1 ; N#Cc1ccc2c(c1)c1cc(C#N)ccc1n2-c1cccc(-c2ccccc2-c2cccc(-n3c4ccccc4c4c5c(ccc43)oc3ccccc35)c2)c1 ; N#Cc1ccc2c(c1)c1cc(C#N)ccc1n2-c1cccc(-c2ccccc2-c2cccc(-n3c4ccccc4c4c5c(ccc43)sc3ccccc35)c2)c1 ; N#Cc1ccc2c(c1)c1cc(C#N)ccc1n2-c1cccc(-c2ccccc2-c2cccc(-n3c4ccccc4c4c5oc6cccnc6c5ccc43)c2)c1]

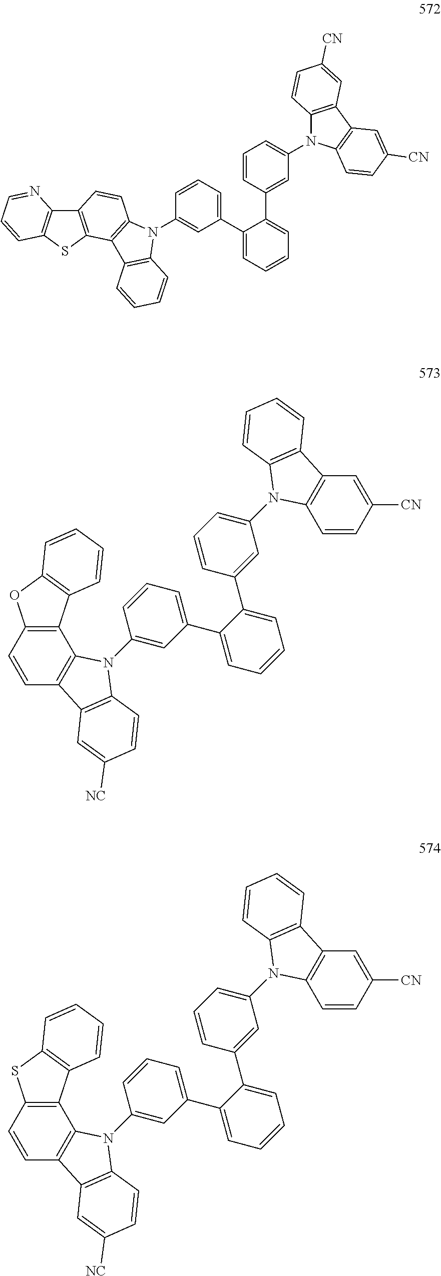 [SMILES: N#Cc1ccc2c(c1)c1cc(C#N)ccc1n2-c1cccc(-c2ccccc2-c2cccc(-n3c4ccccc4c4c5sc6cccnc6c5ccc43)c2)c1 ; N#Cc1ccc2c(c1)c1ccccc1n2-c1cccc(-c2ccccc2-c2cccc(-n3c4ccc(C#N)cc4c4ccc5oc6ccccc6c5c43)c2)c1 ; N#Cc1ccc2c(c1)c1ccccc1n2-c1cccc(-c2ccccc2-c2cccc(-n3c4ccc(C#N)cc4c4ccc5sc6ccccc6c5c43)c2)c1]

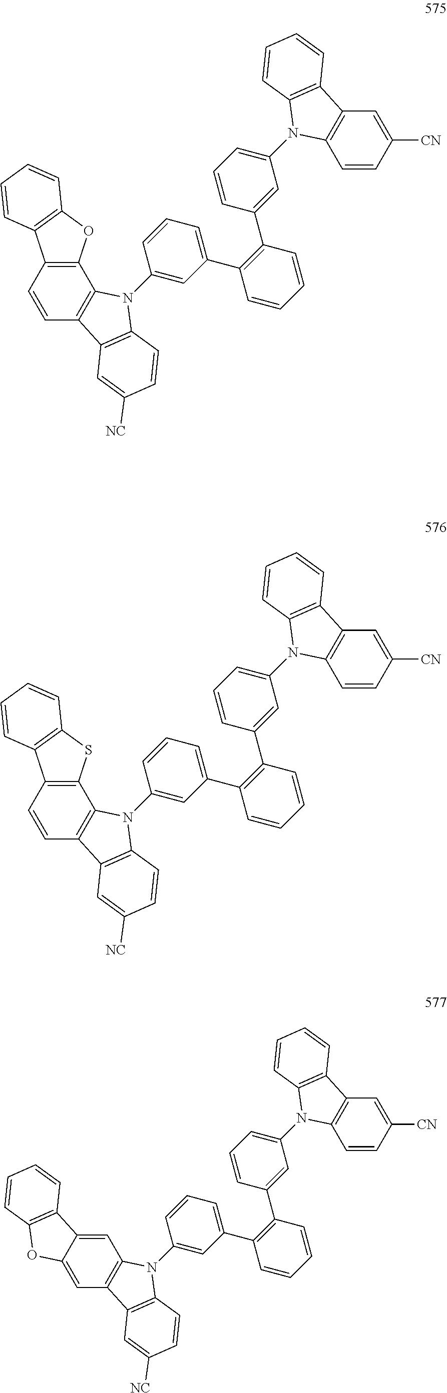 [SMILES: N#Cc1ccc2c(c1)c1ccccc1n2-c1cccc(-c2ccccc2-c2cccc(-n3c4ccc(C#N)cc4c4ccc5c6ccccc6oc5c43)c2)c1 ; N#Cc1ccc2c(c1)c1ccccc1n2-c1cccc(-c2ccccc2-c2cccc(-n3c4ccc(C#N)cc4c4ccc5c6ccccc6sc5c43)c2)c1 ; N#Cc1ccc2c(c1)c1ccccc1n2-c1cccc(-c2ccccc2-c2cccc(-n3c4ccc(C#N)cc4c4cc5oc6ccccc6c5cc43)c2)c1]

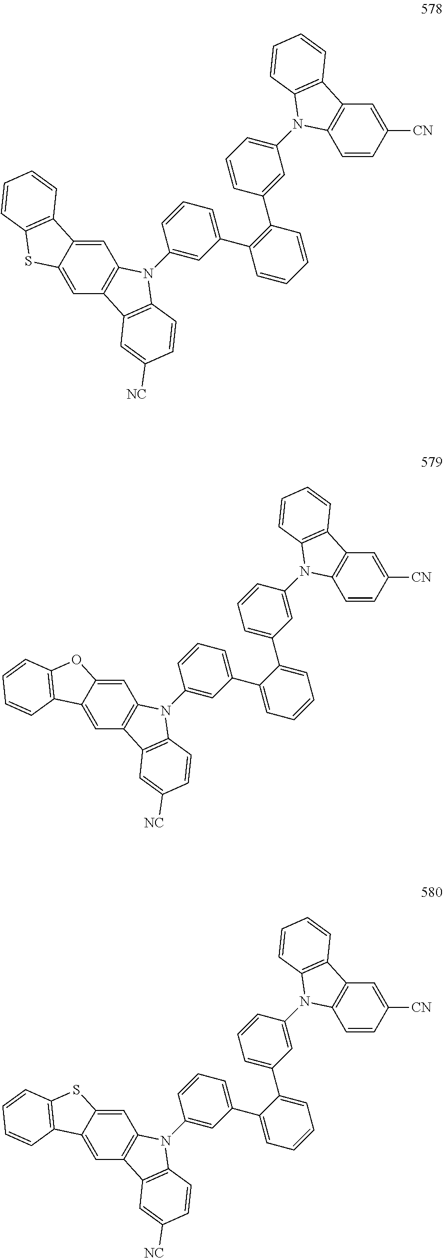 [SMILES: N#Cc1ccc2c(c1)c1ccccc1n2-c1cccc(-c2ccccc2-c2cccc(-n3c4ccc(C#N)cc4c4cc5sc6ccccc6c5cc43)c2)c1 ; N#Cc1ccc2c(c1)c1ccccc1n2-c1cccc(-c2ccccc2-c2cccc(-n3c4ccc(C#N)cc4c4cc5c(cc43)oc3ccccc35)c2)c1 ; N#Cc1ccc2c(c1)c1ccccc1n2-c1cccc(-c2ccccc2-c2cccc(-n3c4ccc(C#N)cc4c4cc5c(cc43)sc3ccccc35)c2)c1]

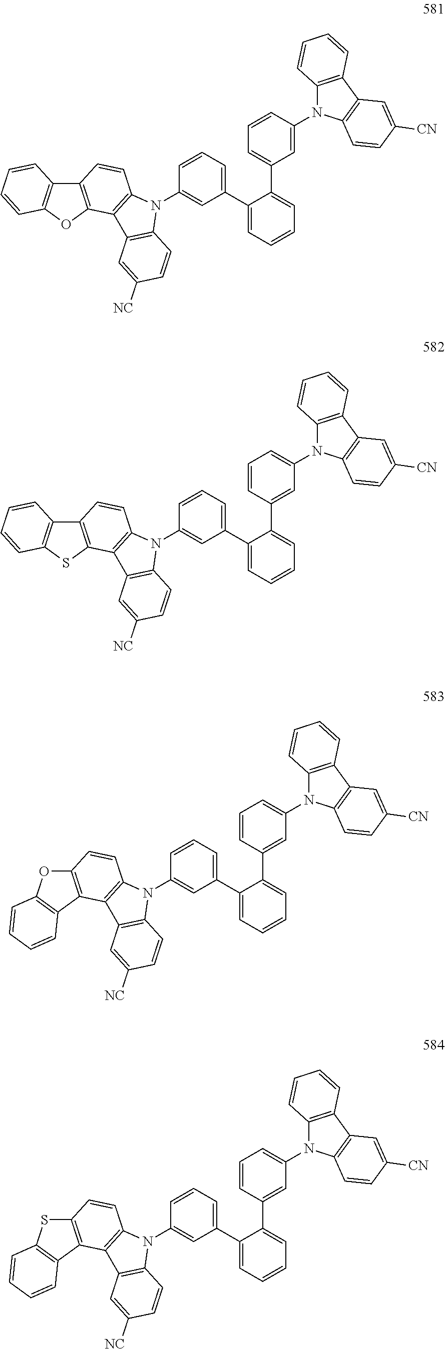 [SMILES: N#Cc1ccc2c(c1)c1ccccc1n2-c1cccc(-c2ccccc2-c2cccc(-n3c4ccc(C#N)cc4c4c5oc6ccccc6c5ccc43)c2)c1 ; N#Cc1ccc2c(c1)c1ccccc1n2-c1cccc(-c2ccccc2-c2cccc(-n3c4ccc(C#N)cc4c4c5sc6ccccc6c5ccc43)c2)c1 ; N#Cc1ccc2c(c1)c1ccccc1n2-c1cccc(-c2ccccc2-c2cccc(-n3c4ccc(C#N)cc4c4c5c(ccc43)oc3ccccc35)c2)c1 ; N#Cc1ccc2c(c1)c1ccccc1n2-c1cccc(-c2ccccc2-c2cccc(-n3c4ccc(C#N)cc4c4c5c(ccc43)sc3ccccc35)c2)c1]

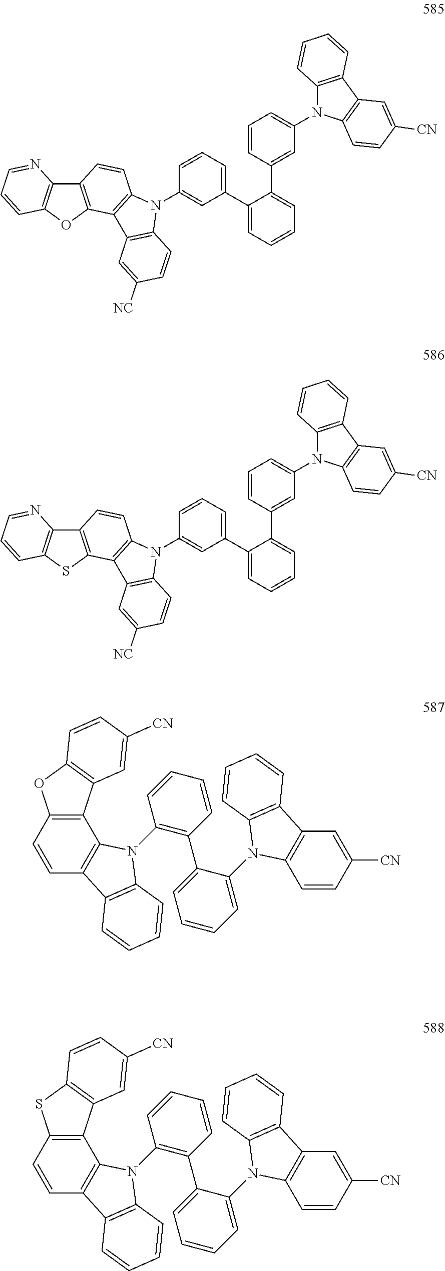 [SMILES: N#Cc1ccc2c(c1)c1ccccc1n2-c1cccc(-c2ccccc2-c2cccc(-n3c4ccc(C#N)cc4c4c5oc6cccnc6c5ccc43)c2)c1 ; N#Cc1ccc2c(c1)c1ccccc1n2-c1cccc(-c2ccccc2-c2cccc(-n3c4ccc(C#N)cc4c4c5sc6cccnc6c5ccc43)c2)c1 ; N#Cc1ccc2oc3ccc4c5ccccc5n(-c5ccccc5-c5ccccc5-n5c6ccccc6c6cc(C#N)ccc65)c4c3c2c1 ; N#Cc1ccc2sc3ccc4c5ccccc5n(-c5ccccc5-c5ccccc5-n5c6ccccc6c6cc(C#N)ccc65)c4c3c2c1]

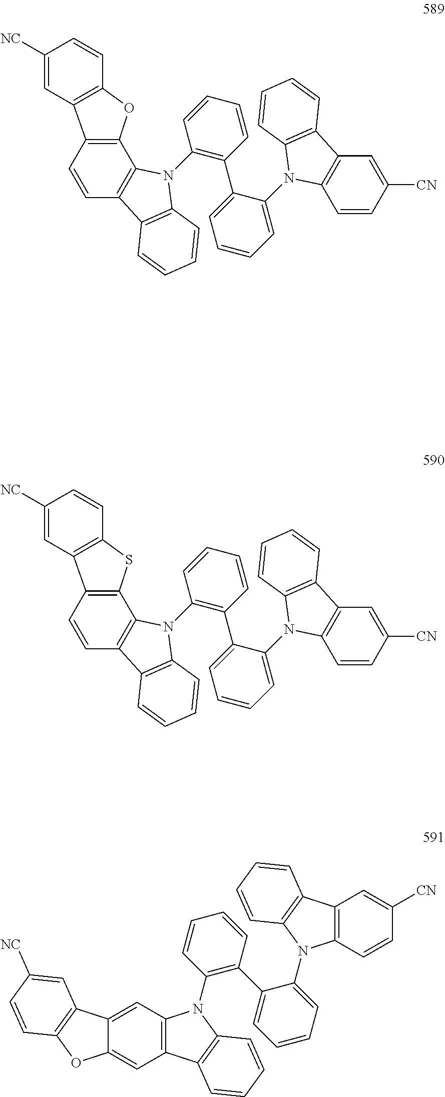 [SMILES: N#Cc1ccc2oc3c(ccc4c5ccccc5n(-c5ccccc5-c5ccccc5-n5c6ccccc6c6cc(C#N)ccc65)c43)c2c1 ; N#Cc1ccc2sc3c(ccc4c5ccccc5n(-c5ccccc5-c5ccccc5-n5c6ccccc6c6cc(C#N)ccc65)c43)c2c1 ; N#Cc1ccc2oc3cc4c5ccccc5n(-c5ccccc5-c5ccccc5-n5c6ccccc6c6cc(C#N)ccc65)c4cc3c2c1]

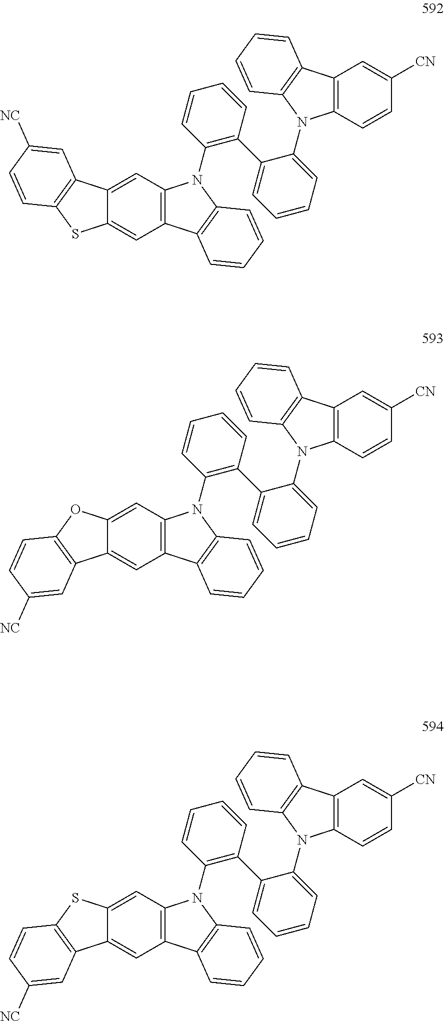 [SMILES: N#Cc1ccc2sc3cc4c5ccccc5n(-c5ccccc5-c5ccccc5-n5c6ccccc6c6cc(C#N)ccc65)c4cc3c2c1 ; N#Cc1ccc2oc3cc4c(cc3c2c1)c1ccccc1n4-c1ccccc1-c1ccccc1-n1c2ccccc2c2cc(C#N)ccc21 ; N#Cc1ccc2sc3cc4c(cc3c2c1)c1ccccc1n4-c1ccccc1-c1ccccc1-n1c2ccccc2c2cc(C#N)ccc21]

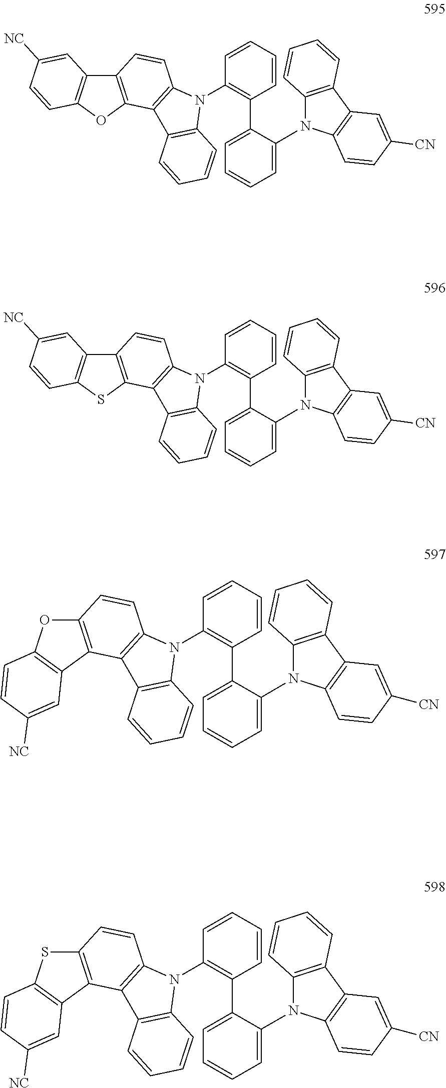 [SMILES: N#Cc1ccc2oc3c(ccc4c3c3ccccc3n4-c3ccccc3-c3ccccc3-n3c4ccccc4c4cc(C#N)ccc43)c2c1 ; N#Cc1ccc2sc3c(ccc4c3c3ccccc3n4-c3ccccc3-c3ccccc3-n3c4ccccc4c4cc(C#N)ccc43)c2c1 ; N#Cc1ccc2oc3ccc4c(c5ccccc5n4-c4ccccc4-c4ccccc4-n4c5ccccc5c5cc(C#N)ccc54)c3c2c1 ; N#Cc1ccc2sc3ccc4c(c5ccccc5n4-c4ccccc4-c4ccccc4-n4c5ccccc5c5cc(C#N)ccc54)c3c2c1]

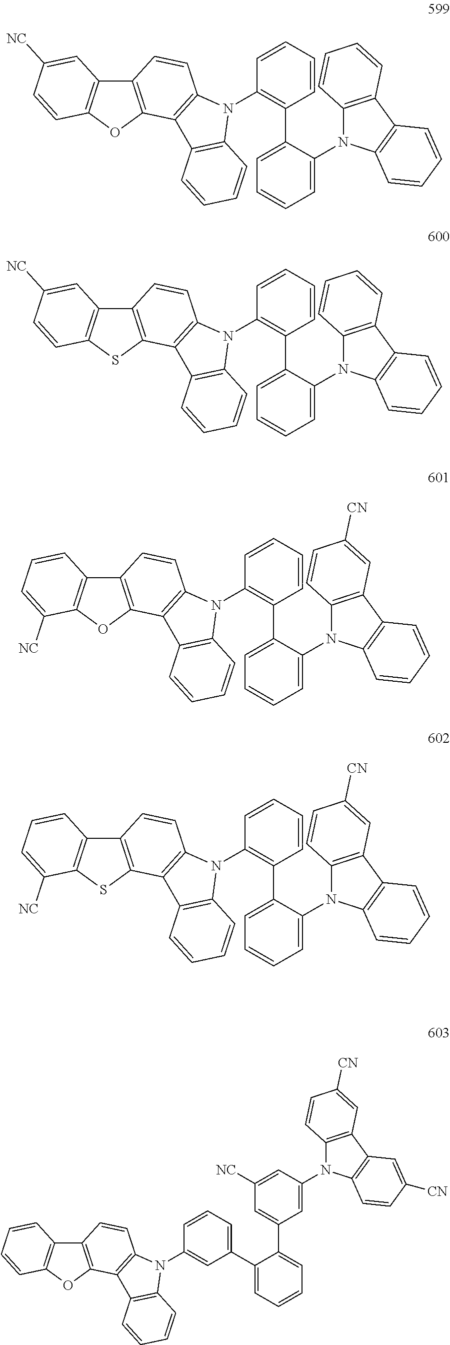 [SMILES: N#Cc1ccc2oc3c(ccc4c3c3ccccc3n4-c3ccccc3-c3ccccc3-n3c4ccccc4c4ccccc43)c2c1 ; N#Cc1ccc2sc3c(ccc4c3c3ccccc3n4-c3ccccc3-c3ccccc3-n3c4ccccc4c4ccccc43)c2c1 ; N#Cc1ccc2c(c1)c1ccccc1n2-c1ccccc1-c1ccccc1-n1c2ccccc2c2c3oc4c(C#N)cccc4c3ccc21 ; N#Cc1ccc2c(c1)c1ccccc1n2-c1ccccc1-c1ccccc1-n1c2ccccc2c2c3sc4c(C#N)cccc4c3ccc21 ; N#Cc1cc(-c2ccccc2-c2cccc(-n3c4ccccc4c4c5oc6ccccc6c5ccc43)c2)cc(-n2c3ccc(C#N)cc3c3cc(C#N)ccc32)c1]

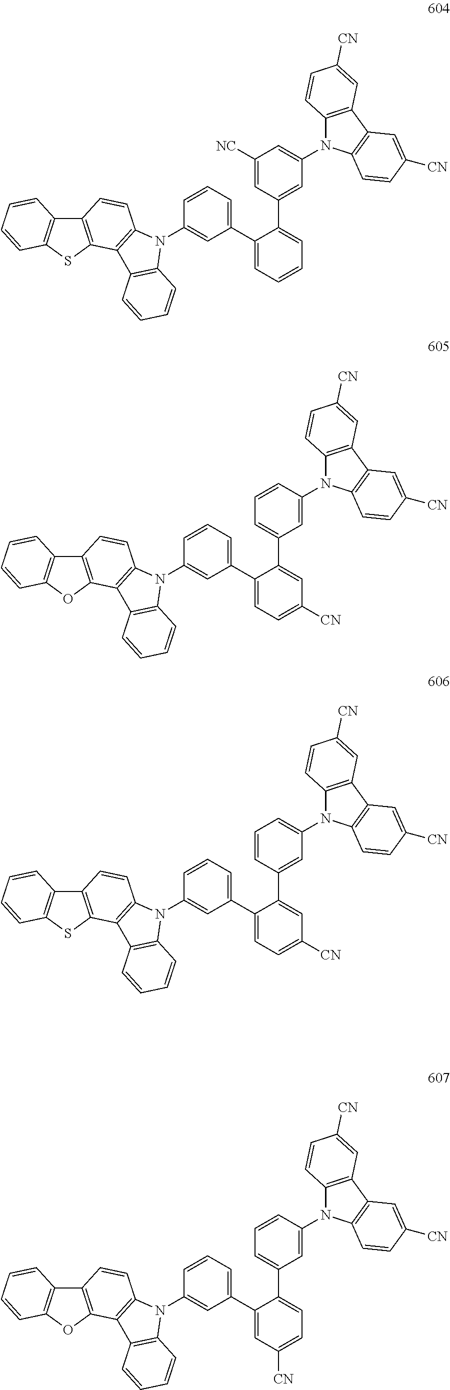 [SMILES: N#Cc1cc(-c2ccccc2-c2cccc(-n3c4ccccc4c4c5sc6ccccc6c5ccc43)c2)cc(-n2c3ccc(C#N)cc3c3cc(C#N)ccc32)c1 ; N#Cc1ccc(-c2cccc(-n3c4ccccc4c4c5oc6ccccc6c5ccc43)c2)c(-c2cccc(-n3c4ccc(C#N)cc4c4cc(C#N)ccc43)c2)c1 ; N#Cc1ccc(-c2cccc(-n3c4ccccc4c4c5sc6ccccc6c5ccc43)c2)c(-c2cccc(-n3c4ccc(C#N)cc4c4cc(C#N)ccc43)c2)c1 ; N#Cc1ccc(-c2cccc(-n3c4ccc(C#N)cc4c4cc(C#N)ccc43)c2)c(-c2cccc(-n3c4ccccc4c4c5oc6ccccc6c5ccc43)c2)c1]

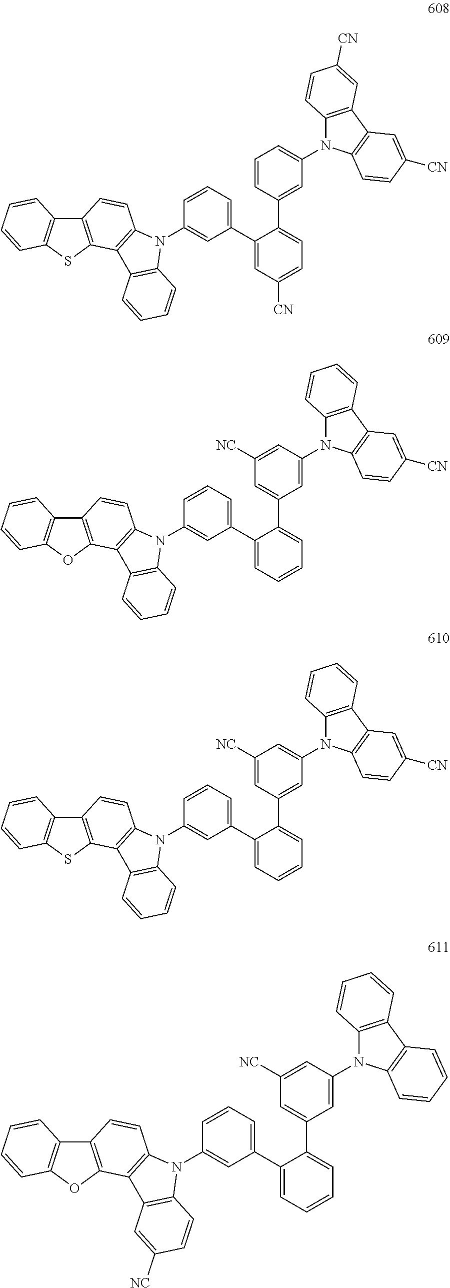 [SMILES: N#Cc1ccc(-c2cccc(-n3c4ccc(C#N)cc4c4cc(C#N)ccc43)c2)c(-c2cccc(-n3c4ccccc4c4c5sc6ccccc6c5ccc43)c2)c1 ; N#Cc1cc(-c2ccccc2-c2cccc(-n3c4ccccc4c4c5oc6ccccc6c5ccc43)c2)cc(-n2c3ccccc3c3cc(C#N)ccc32)c1 ; N#Cc1cc(-c2ccccc2-c2cccc(-n3c4ccccc4c4c5sc6ccccc6c5ccc43)c2)cc(-n2c3ccccc3c3cc(C#N)ccc32)c1 ; N#Cc1cc(-c2ccccc2-c2cccc(-n3c4ccc(C#N)cc4c4c5oc6ccccc6c5ccc43)c2)cc(-n2c3ccccc3c3ccccc32)c1]

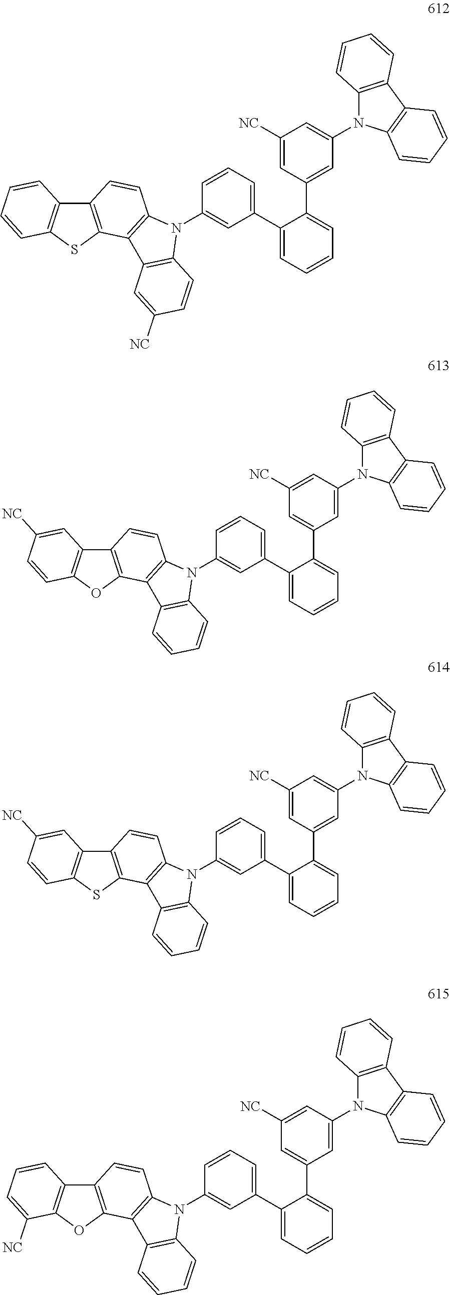 [SMILES: N#Cc1cc(-c2ccccc2-c2cccc(-n3c4ccc(C#N)cc4c4c5sc6ccccc6c5ccc43)c2)cc(-n2c3ccccc3c3ccccc32)c1 ; N#Cc1cc(-c2ccccc2-c2cccc(-n3c4ccccc4c4c5oc6ccc(C#N)cc6c5ccc43)c2)cc(-n2c3ccccc3c3ccccc32)c1 ; N#Cc1cc(-c2ccccc2-c2cccc(-n3c4ccccc4c4c5sc6ccc(C#N)cc6c5ccc43)c2)cc(-n2c3ccccc3c3ccccc32)c1 ; N#Cc1cc(-c2ccccc2-c2cccc(-n3c4ccccc4c4c5oc6c(C#N)cccc6c5ccc43)c2)cc(-n2c3ccccc3c3ccccc32)c1]

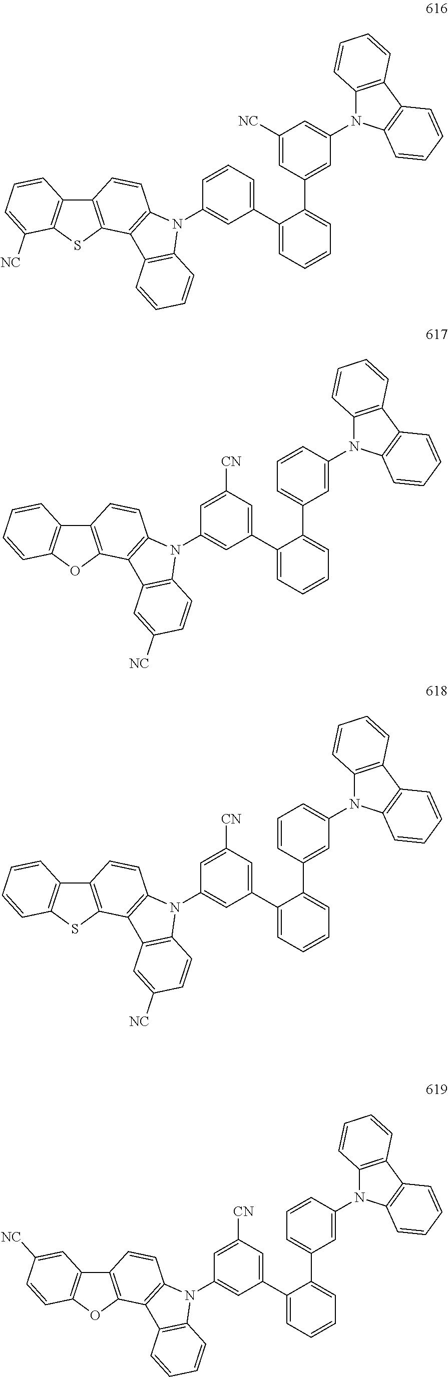 [SMILES: N#Cc1cc(-c2ccccc2-c2cccc(-n3c4ccccc4c4c5sc6c(C#N)cccc6c5ccc43)c2)cc(-n2c3ccccc3c3ccccc32)c1 ; N#Cc1cc(-c2ccccc2-c2cccc(-n3c4ccccc4c4ccccc43)c2)cc(-n2c3ccc(C#N)cc3c3c4oc5ccccc5c4ccc32)c1 ; N#Cc1cc(-c2ccccc2-c2cccc(-n3c4ccccc4c4ccccc43)c2)cc(-n2c3ccc(C#N)cc3c3c4sc5ccccc5c4ccc32)c1 ; N#Cc1cc(-c2ccccc2-c2cccc(-n3c4ccccc4c4ccccc43)c2)cc(-n2c3ccccc3c3c4oc5ccc(C#N)cc5c4ccc32)c1]

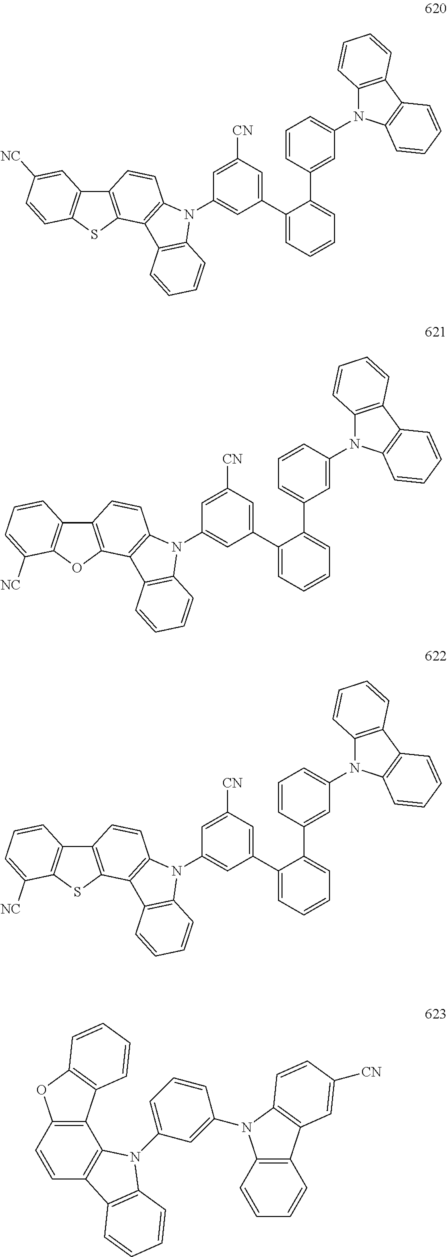 [SMILES: N#Cc1cc(-c2ccccc2-c2cccc(-n3c4ccccc4c4ccccc43)c2)cc(-n2c3ccccc3c3c4sc5ccc(C#N)cc5c4ccc32)c1 ; N#Cc1cc(-c2ccccc2-c2cccc(-n3c4ccccc4c4ccccc43)c2)cc(-n2c3ccccc3c3c4oc5c(C#N)cccc5c4ccc32)c1 ; N#Cc1cc(-c2ccccc2-c2cccc(-n3c4ccccc4c4ccccc43)c2)cc(-n2c3ccccc3c3c4sc5c(C#N)cccc5c4ccc32)c1 ; N#Cc1ccc2c(c1)c1ccccc1n2-c1cccc(-n2c3ccccc3c3ccc4oc5ccccc5c4c32)c1]

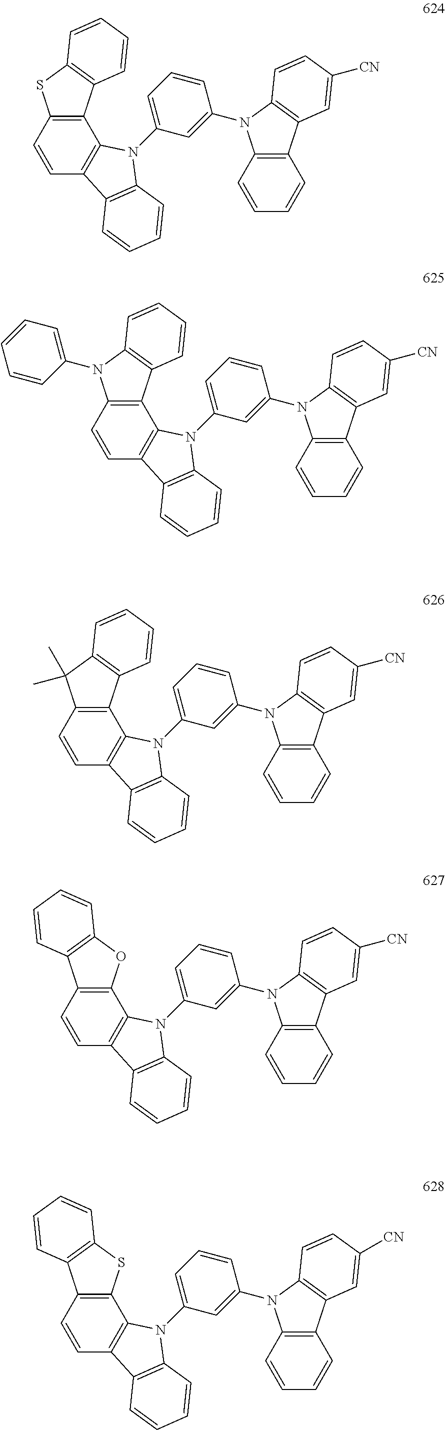 [SMILES: N#Cc1ccc2c(c1)c1ccccc1n2-c1cccc(-n2c3ccccc3c3ccc4sc5ccccc5c4c32)c1 ; N#Cc1ccc2c(c1)c1ccccc1n2-c1cccc(-n2c3ccccc3c3ccc4c(c5ccccc5n4-c4ccccc4)c32)c1 ; CC1(C)c2ccccc2-c2c1ccc1c3ccccc3n(-c3cccc(-n4c5ccccc5c5cc(C#N)ccc54)c3)c21 ; N#Cc1ccc2c(c1)c1ccccc1n2-c1cccc(-n2c3ccccc3c3ccc4c5ccccc5oc4c32)c1 ; N#Cc1ccc2c(c1)c1ccccc1n2-c1cccc(-n2c3ccccc3c3ccc4c5ccccc5sc4c32)c1]

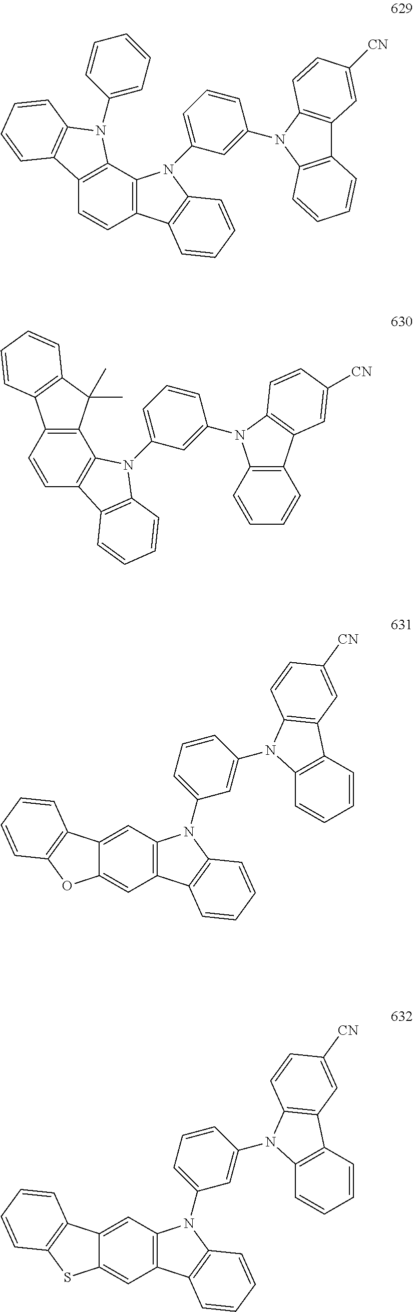 [SMILES: N#Cc1ccc2c(c1)c1ccccc1n2-c1cccc(-n2c3ccccc3c3ccc4c5ccccc5n(-c5ccccc5)c4c32)c1 ; CC1(C)c2ccccc2-c2ccc3c4ccccc4n(-c4cccc(-n5c6ccccc6c6cc(C#N)ccc65)c4)c3c21 ; N#Cc1ccc2c(c1)c1ccccc1n2-c1cccc(-n2c3ccccc3c3cc4oc5ccccc5c4cc32)c1 ; N#Cc1ccc2c(c1)c1ccccc1n2-c1cccc(-n2c3ccccc3c3cc4sc5ccccc5c4cc32)c1]

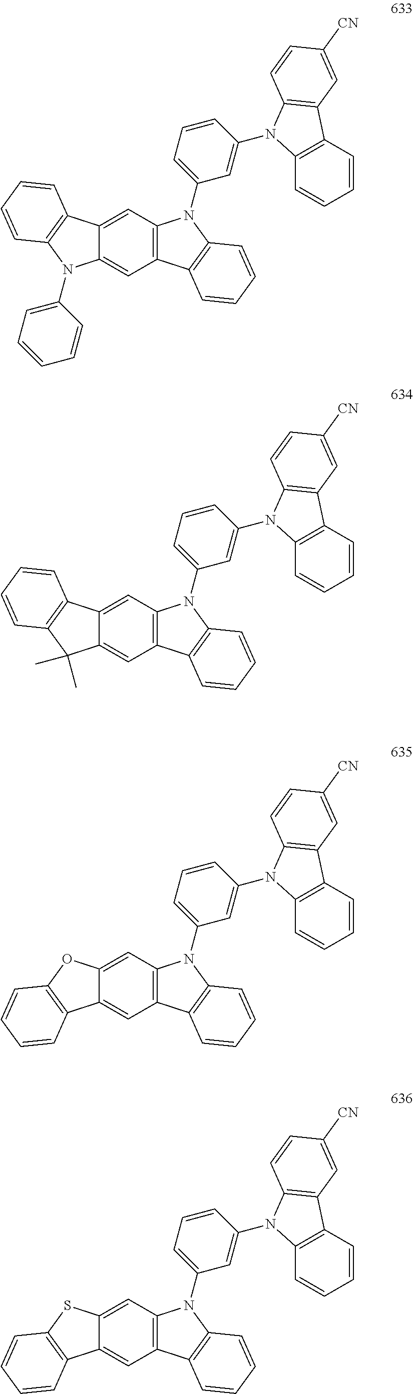 [SMILES: N#Cc1ccc2c(c1)c1ccccc1n2-c1cccc(-n2c3ccccc3c3cc4c(cc32)c2ccccc2n4-c2ccccc2)c1 ; CC1(C)c2ccccc2-c2cc3c(cc21)c1ccccc1n3-c1cccc(-n2c3ccccc3c3cc(C#N)ccc32)c1 ; N#Cc1ccc2c(c1)c1ccccc1n2-c1cccc(-n2c3ccccc3c3cc4c(cc32)oc2ccccc24)c1 ; N#Cc1ccc2c(c1)c1ccccc1n2-c1cccc(-n2c3ccccc3c3cc4c(cc32)sc2ccccc24)c1]

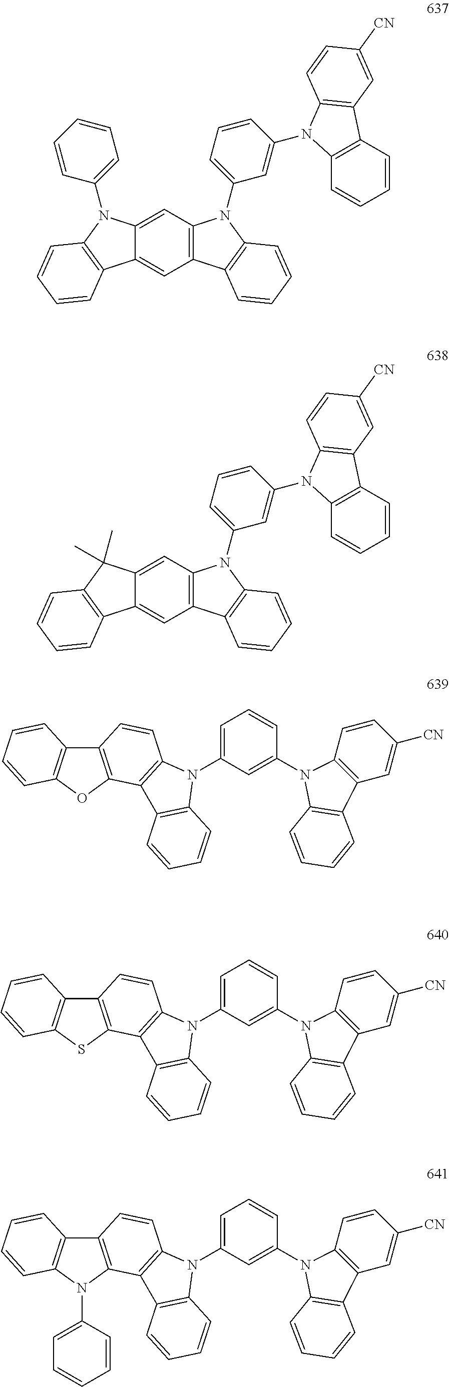 [SMILES: N#Cc1ccc2c(c1)c1ccccc1n2-c1cccc(-n2c3ccccc3c3cc4c5ccccc5n(-c5ccccc5)c4cc32)c1 ; CC1(C)c2ccccc2-c2cc3c4ccccc4n(-c4cccc(-n5c6ccccc6c6cc(C#N)ccc65)c4)c3cc21 ; N#Cc1ccc2c(c1)c1ccccc1n2-c1cccc(-n2c3ccccc3c3c4oc5ccccc5c4ccc32)c1 ; N#Cc1ccc2c(c1)c1ccccc1n2-c1cccc(-n2c3ccccc3c3c4sc5ccccc5c4ccc32)c1 ; N#Cc1ccc2c(c1)c1ccccc1n2-c1cccc(-n2c3ccccc3c3c2ccc2c4ccccc4n(-c4ccccc4)c23)c1]

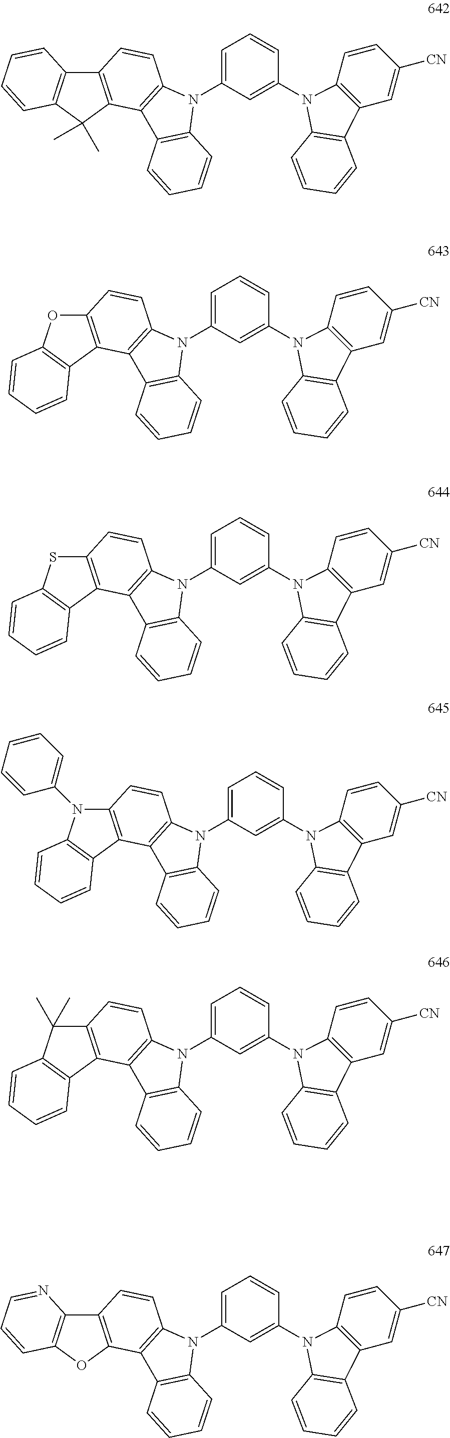 [SMILES: CC1(C)c2ccccc2-c2ccc3c(c21)c1ccccc1n3-c1cccc(-n2c3ccccc3c3cc(C#N)ccc32)c1 ; N#Cc1ccc2c(c1)c1ccccc1n2-c1cccc(-n2c3ccccc3c3c4c(ccc32)oc2ccccc24)c1 ; N#Cc1ccc2c(c1)c1ccccc1n2-c1cccc(-n2c3ccccc3c3c4c(ccc32)sc2ccccc24)c1 ; N#Cc1ccc2c(c1)c1ccccc1n2-c1cccc(-n2c3ccccc3c3c4c5ccccc5n(-c5ccccc5)c4ccc32)c1 ; CC1(C)c2ccccc2-c2c1ccc1c2c2ccccc2n1-c1cccc(-n2c3ccccc3c3cc(C#N)ccc32)c1 ; N#Cc1ccc2c(c1)c1ccccc1n2-c1cccc(-n2c3ccccc3c3c4oc5cccnc5c4ccc32)c1]

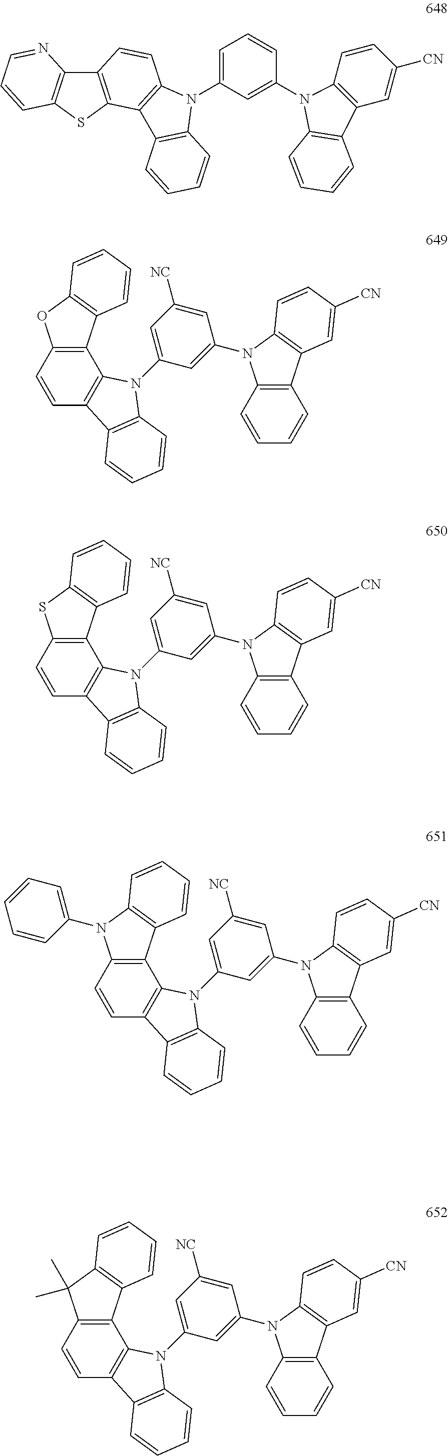 [SMILES: N#Cc1ccc2c(c1)c1ccccc1n2-c1cccc(-n2c3ccccc3c3c4sc5cccnc5c4ccc32)c1 ; N#Cc1cc(-n2c3ccccc3c3cc(C#N)ccc32)cc(-n2c3ccccc3c3ccc4oc5ccccc5c4c32)c1 ; N#Cc1cc(-n2c3ccccc3c3cc(C#N)ccc32)cc(-n2c3ccccc3c3ccc4sc5ccccc5c4c32)c1 ; N#Cc1cc(-n2c3ccccc3c3cc(C#N)ccc32)cc(-n2c3ccccc3c3ccc4c(c5ccccc5n4-c4ccccc4)c32)c1 ; CC1(C)c2ccccc2-c2c1ccc1c3ccccc3n(-c3cc(C#N)cc(-n4c5ccccc5c5cc(C#N)ccc54)c3)c21]

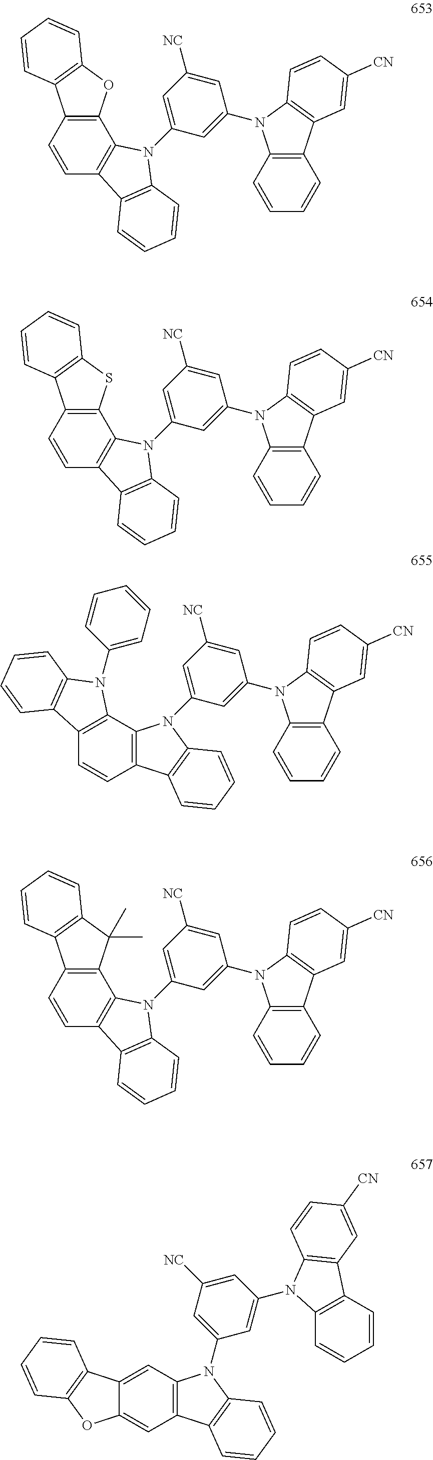

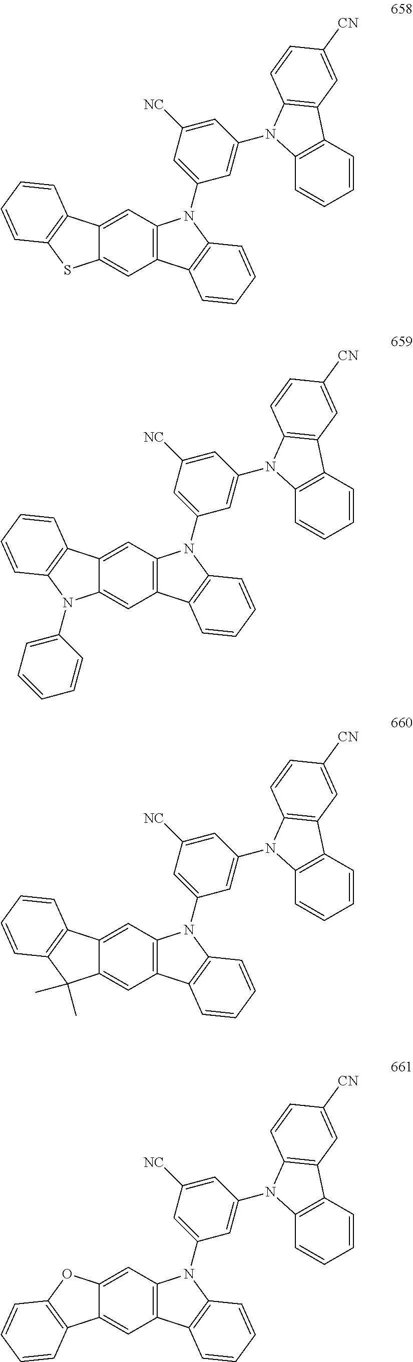 [SMILES: N#Cc1cc(-n2c3ccccc3c3cc(C#N)ccc32)cc(-n2c3ccccc3c3cc4sc5ccccc5c4cc32)c1 ; N#Cc1cc(-n2c3ccccc3c3cc(C#N)ccc32)cc(-n2c3ccccc3c3cc4c(cc32)c2ccccc2n4-c2ccccc2)c1 ; CC1(C)c2ccccc2-c2cc3c(cc21)c1ccccc1n3-c1cc(C#N)cc(-n2c3ccccc3c3cc(C#N)ccc32)c1 ; N#Cc1cc(-n2c3ccccc3c3cc(C#N)ccc32)cc(-n2c3ccccc3c3cc4c(cc32)oc2ccccc24)c1]

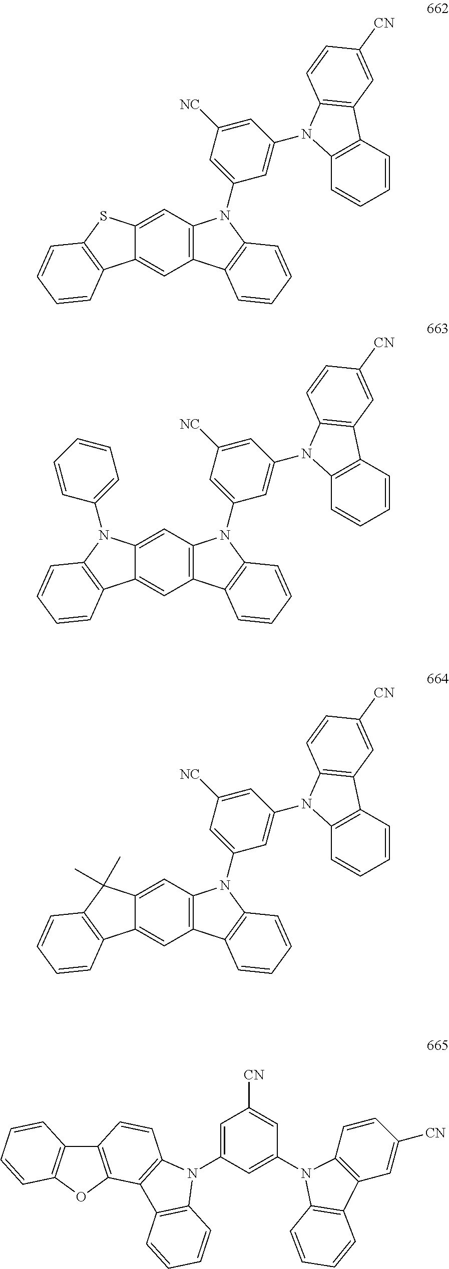 [SMILES: N#Cc1cc(-n2c3ccccc3c3cc(C#N)ccc32)cc(-n2c3ccccc3c3cc4c(cc32)sc2ccccc24)c1 ; N#Cc1cc(-n2c3ccccc3c3cc(C#N)ccc32)cc(-n2c3ccccc3c3cc4c5ccccc5n(-c5ccccc5)c4cc32)c1 ; CC1(C)c2ccccc2-c2cc3c4ccccc4n(-c4cc(C#N)cc(-n5c6ccccc6c6cc(C#N)ccc65)c4)c3cc21 ; N#Cc1cc(-n2c3ccccc3c3cc(C#N)ccc32)cc(-n2c3ccccc3c3c4oc5ccccc5c4ccc32)c1]

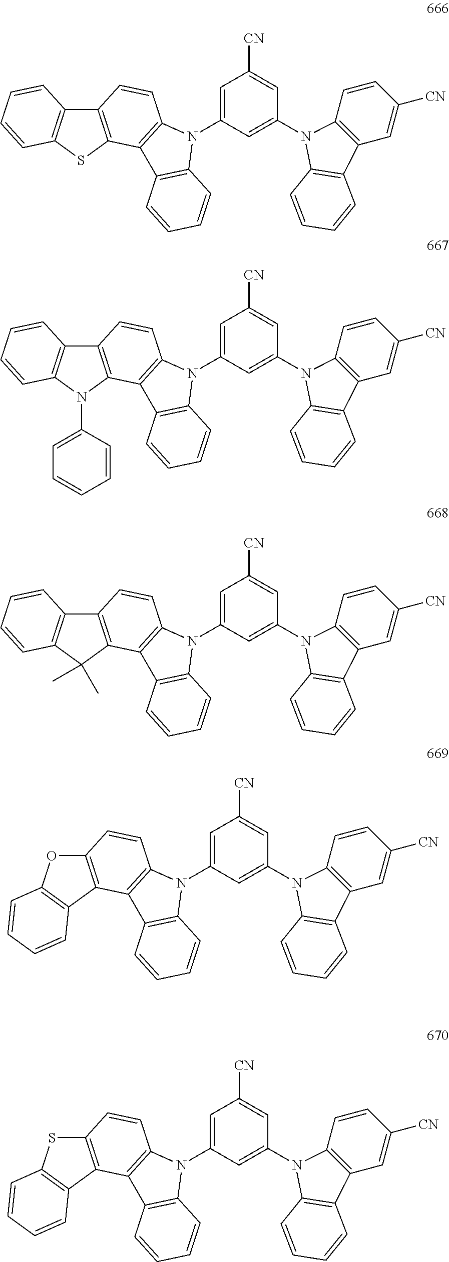 [SMILES: N#Cc1cc(-n2c3ccccc3c3cc(C#N)ccc32)cc(-n2c3ccccc3c3c4sc5ccccc5c4ccc32)c1 ; N#Cc1cc(-n2c3ccccc3c3cc(C#N)ccc32)cc(-n2c3ccccc3c3c2ccc2c4ccccc4n(-c4ccccc4)c23)c1 ; CC1(C)c2ccccc2-c2ccc3c(c21)c1ccccc1n3-c1cc(C#N)cc(-n2c3ccccc3c3cc(C#N)ccc32)c1 ; N#Cc1cc(-n2c3ccccc3c3cc(C#N)ccc32)cc(-n2c3ccccc3c3c4c(ccc32)oc2ccccc24)c1 ; N#Cc1cc(-n2c3ccccc3c3cc(C#N)ccc32)cc(-n2c3ccccc3c3c4c(ccc32)sc2ccccc24)c1]

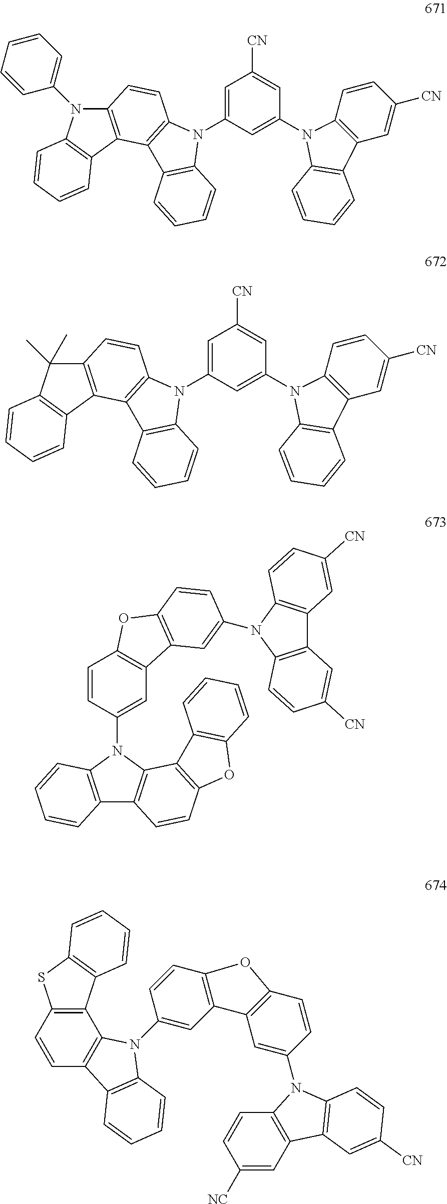 [SMILES: N#Cc1cc(-n2c3ccccc3c3cc(C#N)ccc32)cc(-n2c3ccccc3c3c4c5ccccc5n(-c5ccccc5)c4ccc32)c1 ; CC1(C)c2ccccc2-c2c1ccc1c2c2ccccc2n1-c1cc(C#N)cc(-n2c3ccccc3c3cc(C#N)ccc32)c1 ; N#Cc1ccc2c(c1)c1cc(C#N)ccc1n2-c1ccc2oc3ccc(-n4c5ccccc5c5ccc6oc7ccccc7c6c54)cc3c2c1 ; N#Cc1ccc2c(c1)c1cc(C#N)ccc1n2-c1ccc2oc3ccc(-n4c5ccccc5c5ccc6sc7ccccc7c6c54)cc3c2c1]

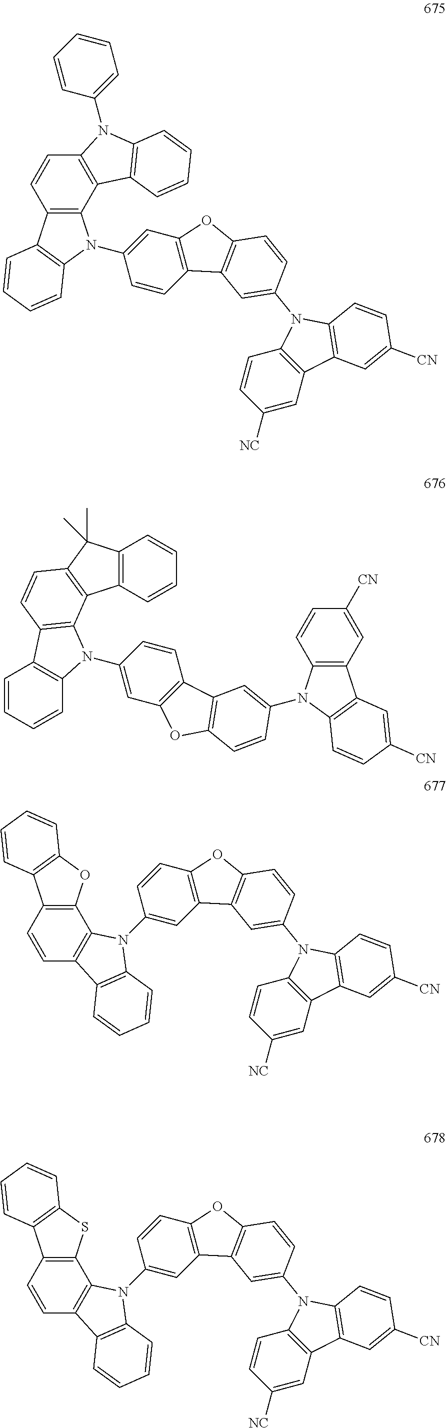 [SMILES: N#Cc1ccc2c(c1)c1cc(C#N)ccc1n2-c1ccc2oc3cc(-n4c5ccccc5c5ccc6c(c7ccccc7n6-c6ccccc6)c54)ccc3c2c1 ; CC1(C)c2ccccc2-c2c1ccc1c3ccccc3n(-c3ccc4c(c3)oc3ccc(-n5c6ccc(C#N)cc6c6cc(C#N)ccc65)cc34)c21 ; N#Cc1ccc2c(c1)c1cc(C#N)ccc1n2-c1ccc2oc3ccc(-n4c5ccccc5c5ccc6c7ccccc7oc6c54)cc3c2c1 ; N#Cc1ccc2c(c1)c1cc(C#N)ccc1n2-c1ccc2oc3ccc(-n4c5ccccc5c5ccc6c7ccccc7sc6c54)cc3c2c1]

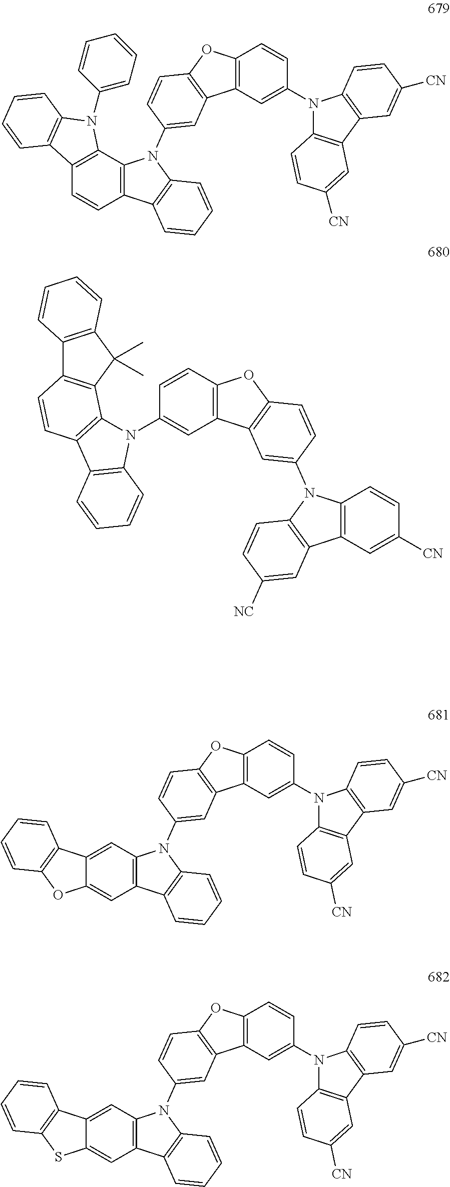 [SMILES: N#Cc1ccc2c(c1)c1cc(C#N)ccc1n2-c1ccc2oc3ccc(-n4c5ccccc5c5ccc6c7ccccc7n(-c7ccccc7)c6c54)cc3c2c1 ; CC1(C)c2ccccc2-c2ccc3c4ccccc4n(-c4ccc5oc6ccc(-n7c8ccc(C#N)cc8c8cc(C#N)ccc87)cc6c5c4)c3c21 ; N#Cc1ccc2c(c1)c1cc(C#N)ccc1n2-c1ccc2oc3ccc(-n4c5ccccc5c5cc6oc7ccccc7c6cc54)cc3c2c1 ; N#Cc1ccc2c(c1)c1cc(C#N)ccc1n2-c1ccc2oc3ccc(-n4c5ccccc5c5cc6sc7ccccc7c6cc54)cc3c2c1]

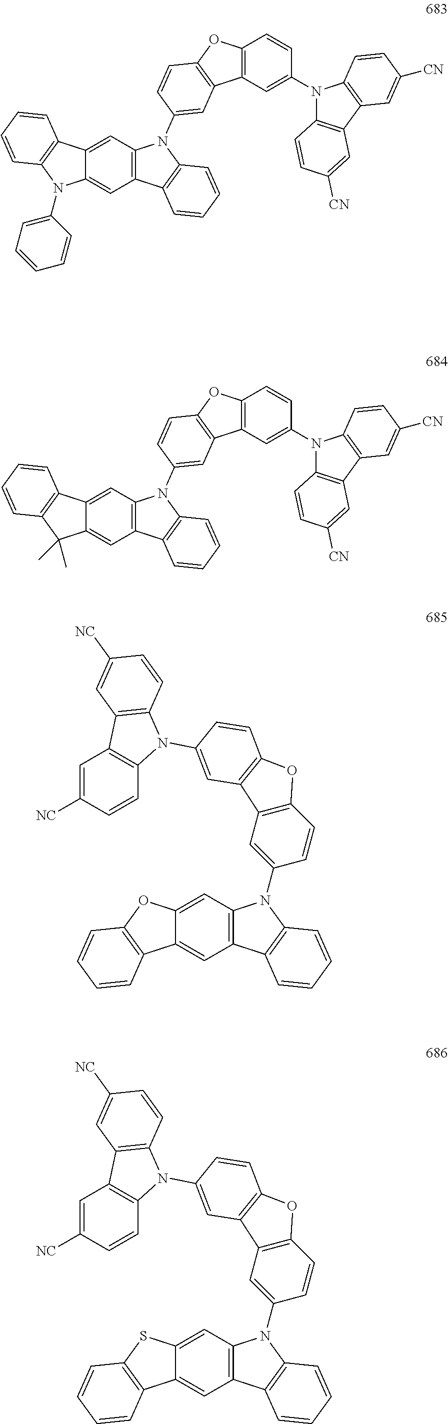 [SMILES: N#Cc1ccc2c(c1)c1cc(C#N)ccc1n2-c1ccc2oc3ccc(-n4c5ccccc5c5cc6c(cc54)c4ccccc4n6-c4ccccc4)cc3c2c1 ; CC1(C)c2ccccc2-c2cc3c(cc21)c1ccccc1n3-c1ccc2oc3ccc(-n4c5ccc(C#N)cc5c5cc(C#N)ccc54)cc3c2c1 ; N#Cc1ccc2c(c1)c1cc(C#N)ccc1n2-c1ccc2oc3ccc(-n4c5ccccc5c5cc6c(cc54)oc4ccccc46)cc3c2c1 ; N#Cc1ccc2c(c1)c1cc(C#N)ccc1n2-c1ccc2oc3ccc(-n4c5ccccc5c5cc6c(cc54)sc4ccccc46)cc3c2c1]

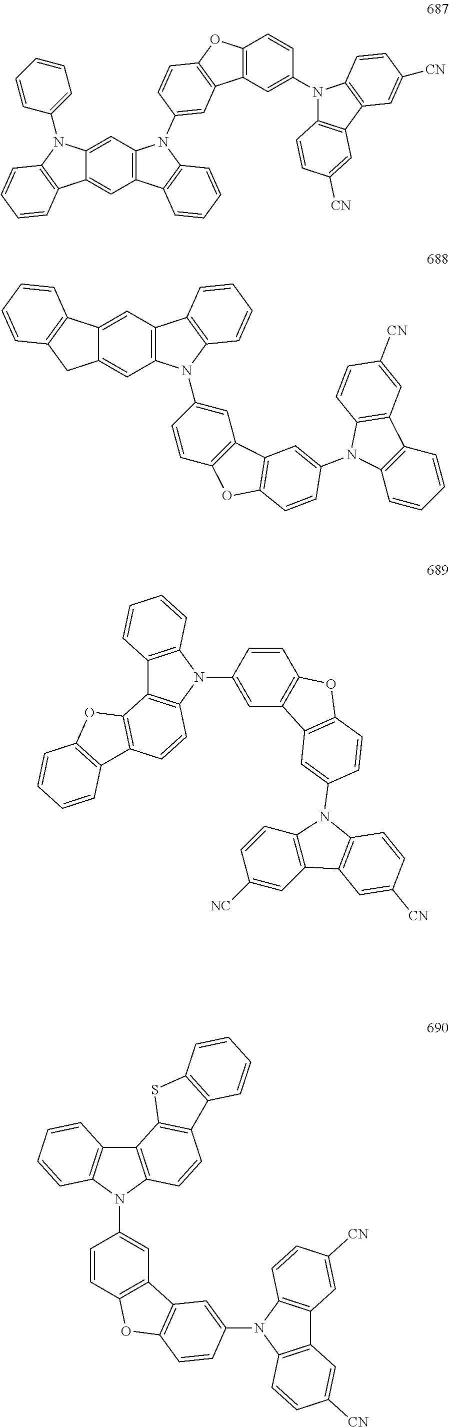 [SMILES: N#Cc1ccc2c(c1)c1cc(C#N)ccc1n2-c1ccc2oc3ccc(-n4c5ccccc5c5cc6c7ccccc7n(-c7ccccc7)c6cc54)cc3c2c1 ; N#Cc1ccc2c(c1)c1ccccc1n2-c1ccc2oc3ccc(-n4c5ccccc5c5cc6c(cc54)Cc4ccccc4-6)cc3c2c1 ; N#Cc1ccc2c(c1)c1cc(C#N)ccc1n2-c1ccc2oc3ccc(-n4c5ccccc5c5c6oc7ccccc7c6ccc54)cc3c2c1 ; N#Cc1ccc2c(c1)c1cc(C#N)ccc1n2-c1ccc2oc3ccc(-n4c5ccccc5c5c6sc7ccccc7c6ccc54)cc3c2c1]

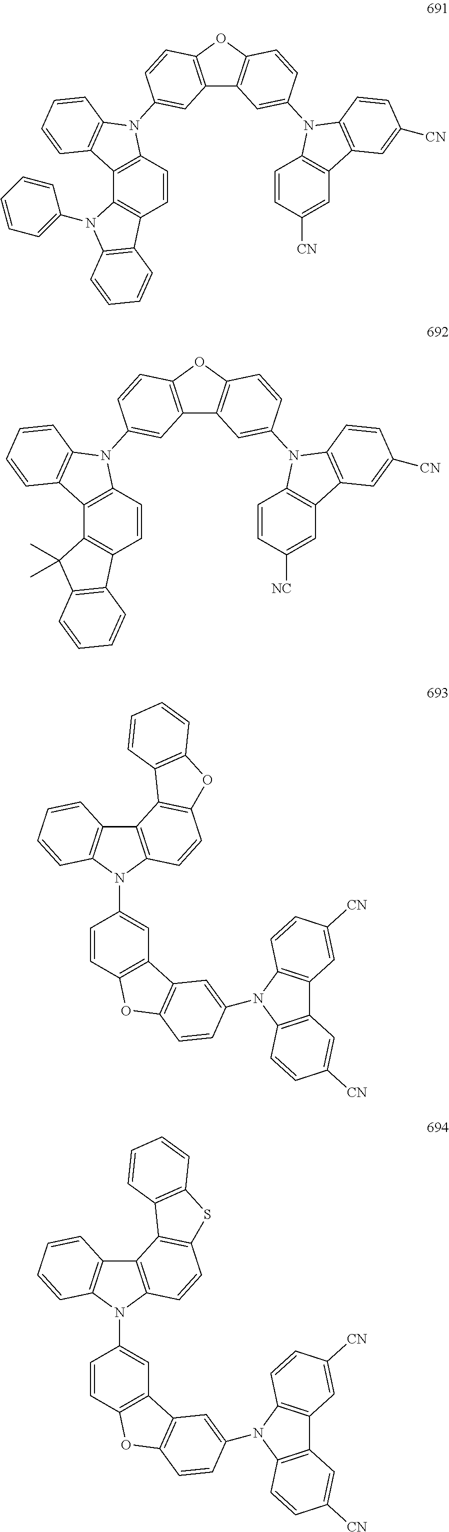 [SMILES: N#Cc1ccc2c(c1)c1cc(C#N)ccc1n2-c1ccc2oc3ccc(-n4c5ccccc5c5c4ccc4c6ccccc6n(-c6ccccc6)c45)cc3c2c1 ; CC1(C)c2ccccc2-c2ccc3c(c21)c1ccccc1n3-c1ccc2oc3ccc(-n4c5ccc(C#N)cc5c5cc(C#N)ccc54)cc3c2c1 ; N#Cc1ccc2c(c1)c1cc(C#N)ccc1n2-c1ccc2oc3ccc(-n4c5ccccc5c5c6c(ccc54)oc4ccccc46)cc3c2c1 ; N#Cc1ccc2c(c1)c1cc(C#N)ccc1n2-c1ccc2oc3ccc(-n4c5ccccc5c5c6c(ccc54)sc4ccccc46)cc3c2c1]

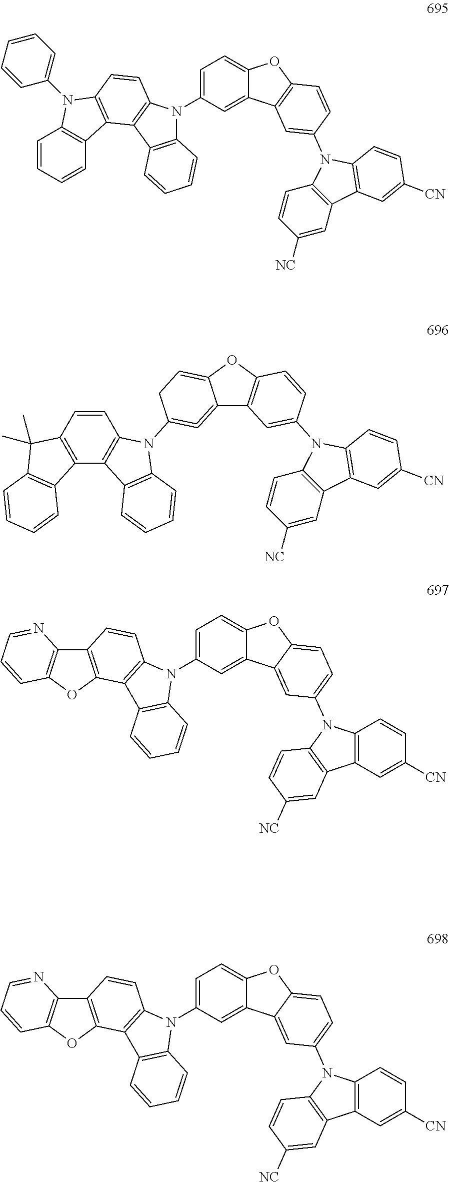 [SMILES: N#Cc1ccc2c(c1)c1cc(C#N)ccc1n2-c1ccc2oc3ccc(-n4c5ccccc5c5c6c7ccccc7n(-c7ccccc7)c6ccc54)cc3c2c1 ; CC1(C)c2ccccc2-c2c1ccc1c2c2ccccc2n1C1=C=c2c(oc3ccc(-n4c5ccc(C#N)cc5c5cc(C#N)ccc54)cc23)=CC1 ; N#Cc1ccc2c(c1)c1cc(C#N)ccc1n2-c1ccc2oc3ccc(-n4c5ccccc5c5c6oc7cccnc7c6ccc54)cc3c2c1 ; N#Cc1ccc2c(c1)c1cc(C#N)ccc1n2-c1ccc2oc3ccc(-n4c5ccccc5c5c6oc7cccnc7c6ccc54)cc3c2c1]

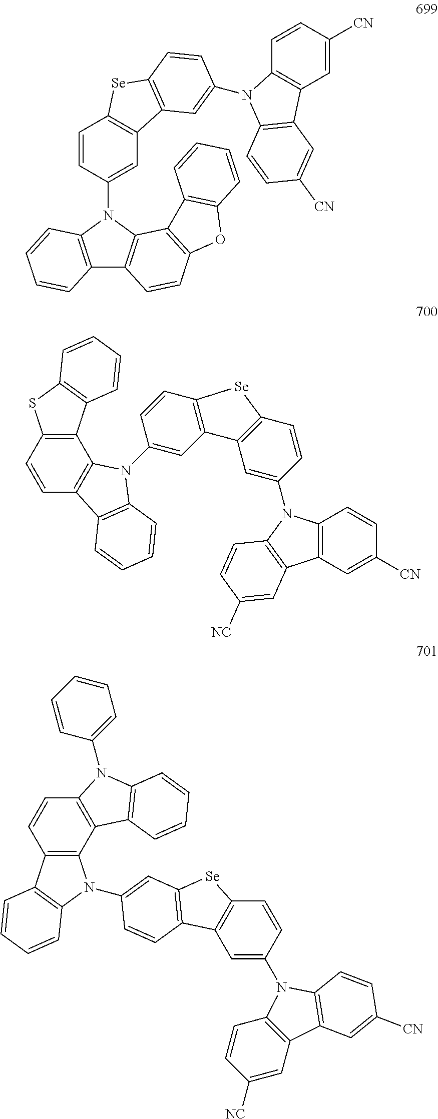 [SMILES: N#Cc1ccc2c(c1)c1cc(C#N)ccc1n2-c1ccc2[se]c3ccc(-n4c5ccccc5c5ccc6oc7ccccc7c6c54)cc3c2c1 ; N#Cc1ccc2c(c1)c1cc(C#N)ccc1n2-c1ccc2[se]c3ccc(-n4c5ccccc5c5ccc6sc7ccccc7c6c54)cc3c2c1 ; N#Cc1ccc2c(c1)c1cc(C#N)ccc1n2-c1ccc2[se]c3cc(-n4c5ccccc5c5ccc6c(c7ccccc7n6-c6ccccc6)c54)ccc3c2c1]

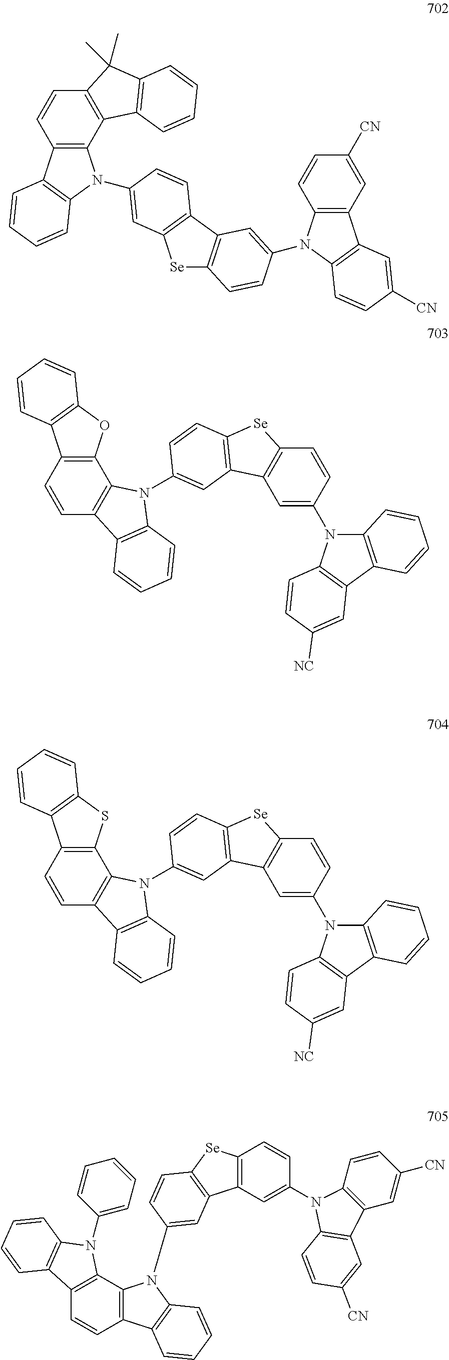 [SMILES: CC1(C)c2ccccc2-c2c1ccc1c3ccccc3n(-c3ccc4c(c3)[se]c3ccc(-n5c6ccc(C#N)cc6c6cc(C#N)ccc65)cc34)c21 ; N#Cc1ccc2c(c1)c1ccccc1n2-c1ccc2[se]c3ccc(-n4c5ccccc5c5ccc6c7ccccc7oc6c54)cc3c2c1 ; N#Cc1ccc2c(c1)c1ccccc1n2-c1ccc2[se]c3ccc(-n4c5ccccc5c5ccc6c7ccccc7sc6c54)cc3c2c1 ; N#Cc1ccc2c(c1)c1cc(C#N)ccc1n2-c1ccc2[se]c3ccc(-n4c5ccccc5c5ccc6c7ccccc7n(-c7ccccc7)c6c54)cc3c2c1]

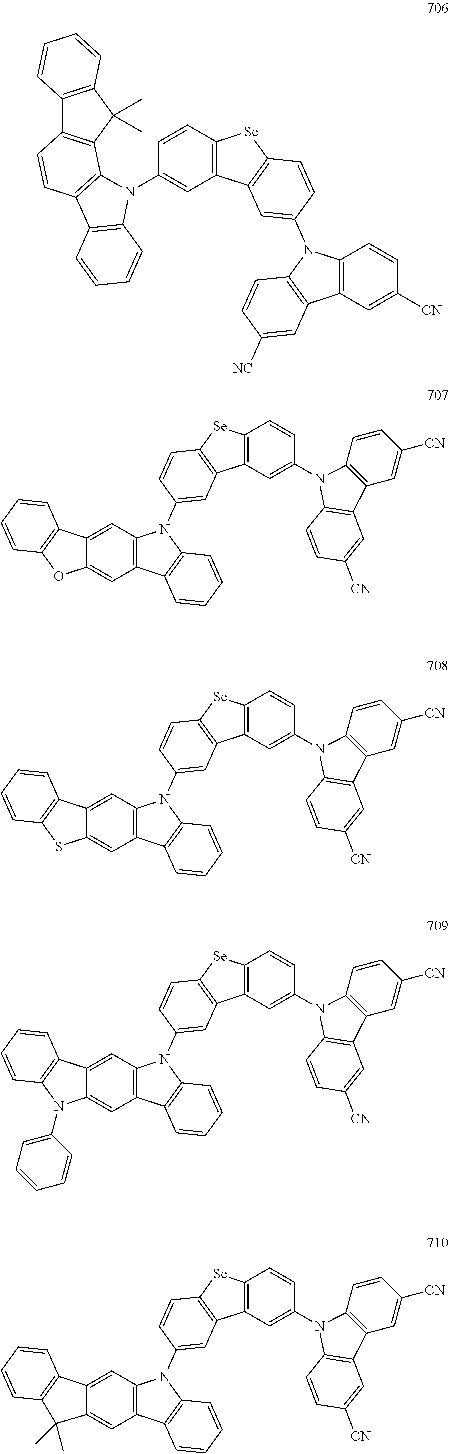 [SMILES: CC1(C)c2ccccc2-c2ccc3c4ccccc4n(-c4ccc5[se]c6ccc(-n7c8ccc(C#N)cc8c8cc(C#N)ccc87)cc6c5c4)c3c21 ; N#Cc1ccc2c(c1)c1cc(C#N)ccc1n2-c1ccc2[se]c3ccc(-n4c5ccccc5c5cc6oc7ccccc7c6cc54)cc3c2c1 ; N#Cc1ccc2c(c1)c1cc(C#N)ccc1n2-c1ccc2[se]c3ccc(-n4c5ccccc5c5cc6sc7ccccc7c6cc54)cc3c2c1 ; N#Cc1ccc2c(c1)c1cc(C#N)ccc1n2-c1ccc2[se]c3ccc(-n4c5ccccc5c5cc6c(cc54)c4ccccc4n6-c4ccccc4)cc3c2c1 ; CC1(C)c2ccccc2-c2cc3c(cc21)c1ccccc1n3-c1ccc2[se]c3ccc(-n4c5ccc(C#N)cc5c5cc(C#N)ccc54)cc3c2c1]

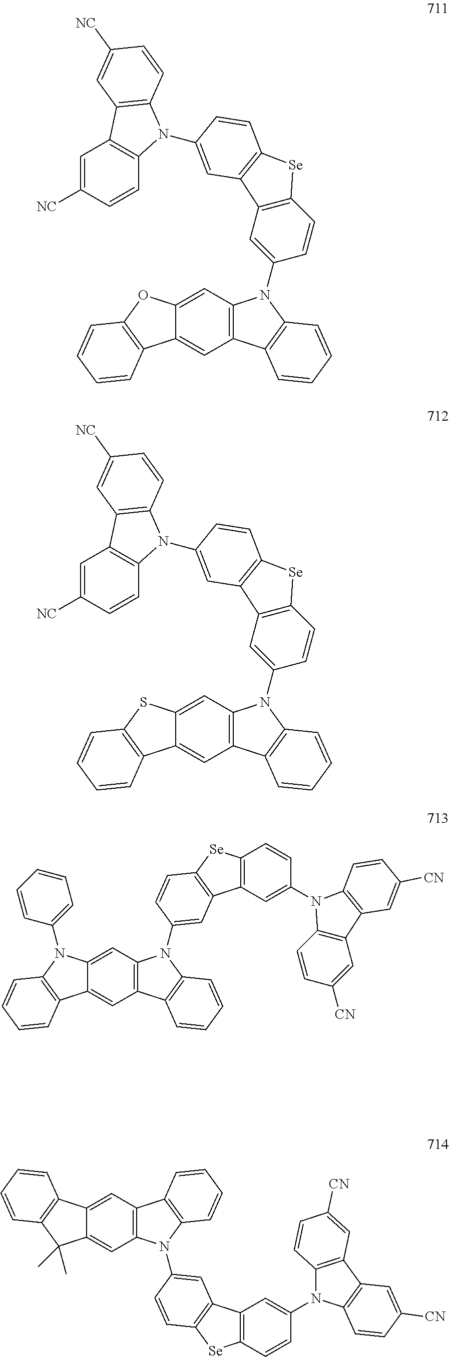 [SMILES: N#Cc1ccc2c(c1)c1cc(C#N)ccc1n2-c1ccc2[se]c3ccc(-n4c5ccccc5c5cc6c(cc54)oc4ccccc46)cc3c2c1 ; N#Cc1ccc2c(c1)c1cc(C#N)ccc1n2-c1ccc2[se]c3ccc(-n4c5ccccc5c5cc6c(cc54)sc4ccccc46)cc3c2c1 ; N#Cc1ccc2c(c1)c1cc(C#N)ccc1n2-c1ccc2[se]c3ccc(-n4c5ccccc5c5cc6c7ccccc7n(-c7ccccc7)c6cc54)cc3c2c1 ; CC1(C)c2ccccc2-c2cc3c4ccccc4n(-c4ccc5[se]c6ccc(-n7c8ccc(C#N)cc8c8cc(C#N)ccc87)cc6c5c4)c3cc21]

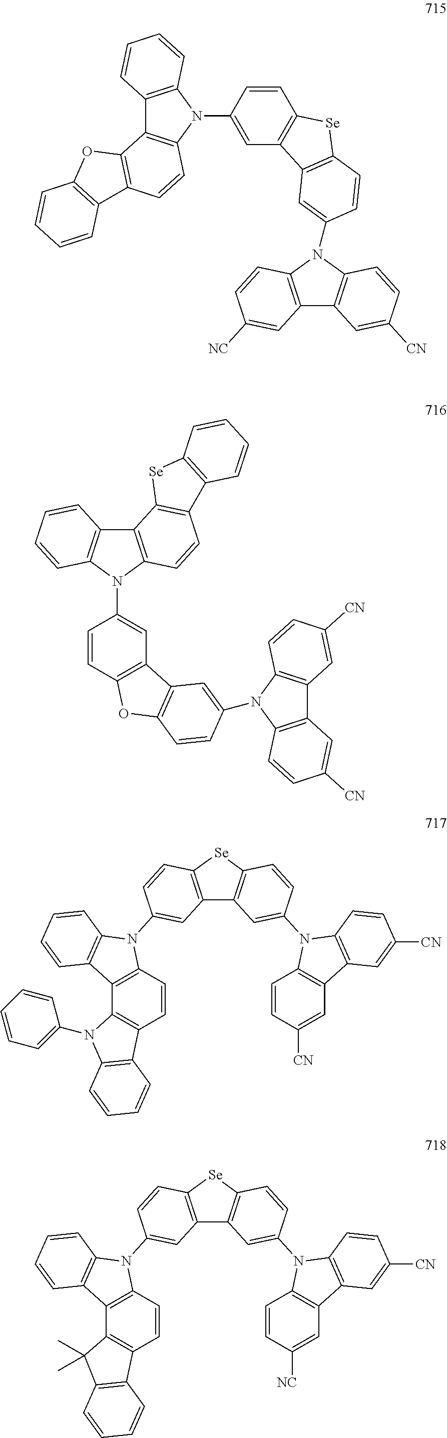 [SMILES: N#Cc1ccc2c(c1)c1cc(C#N)ccc1n2-c1ccc2[se]c3ccc(-n4c5ccccc5c5c6oc7ccccc7c6ccc54)cc3c2c1 ; N#Cc1ccc2c(c1)c1cc(C#N)ccc1n2-c1ccc2oc3ccc(-n4c5ccccc5c5c6[se]c7ccccc7c6ccc54)cc3c2c1 ; N#Cc1ccc2c(c1)c1cc(C#N)ccc1n2-c1ccc2[se]c3ccc(-n4c5ccccc5c5c4ccc4c6ccccc6n(-c6ccccc6)c45)cc3c2c1 ; CC1(C)c2ccccc2-c2ccc3c(c21)c1ccccc1n3-c1ccc2[se]c3ccc(-n4c5ccc(C#N)cc5c5cc(C#N)ccc54)cc3c2c1]

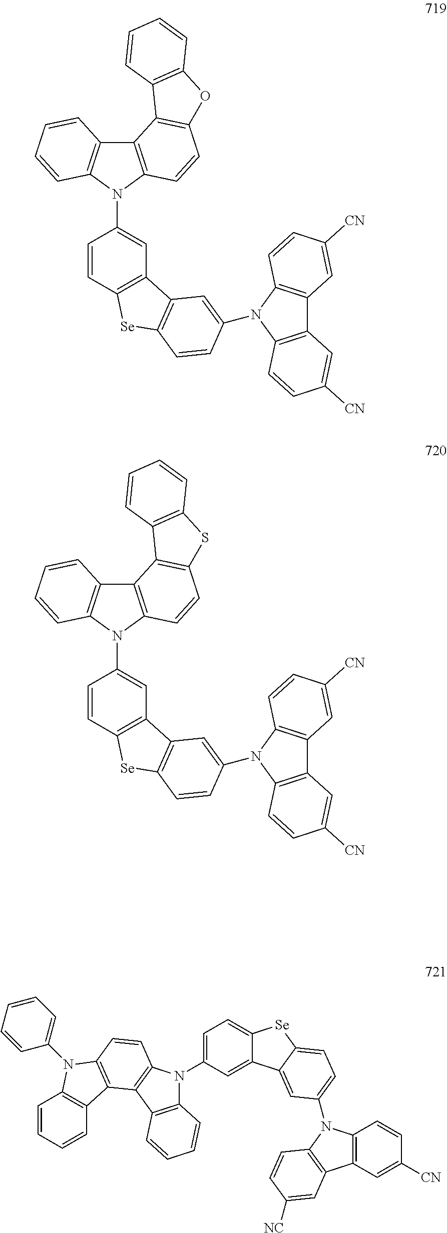 [SMILES: N#Cc1ccc2c(c1)c1cc(C#N)ccc1n2-c1ccc2[se]c3ccc(-n4c5ccccc5c5c6c(ccc54)oc4ccccc46)cc3c2c1 ; N#Cc1ccc2c(c1)c1cc(C#N)ccc1n2-c1ccc2[se]c3ccc(-n4c5ccccc5c5c6c(ccc54)sc4ccccc46)cc3c2c1 ; N#Cc1ccc2c(c1)c1cc(C#N)ccc1n2-c1ccc2[se]c3ccc(-n4c5ccccc5c5c6c7ccccc7n(-c7ccccc7)c6ccc54)cc3c2c1]

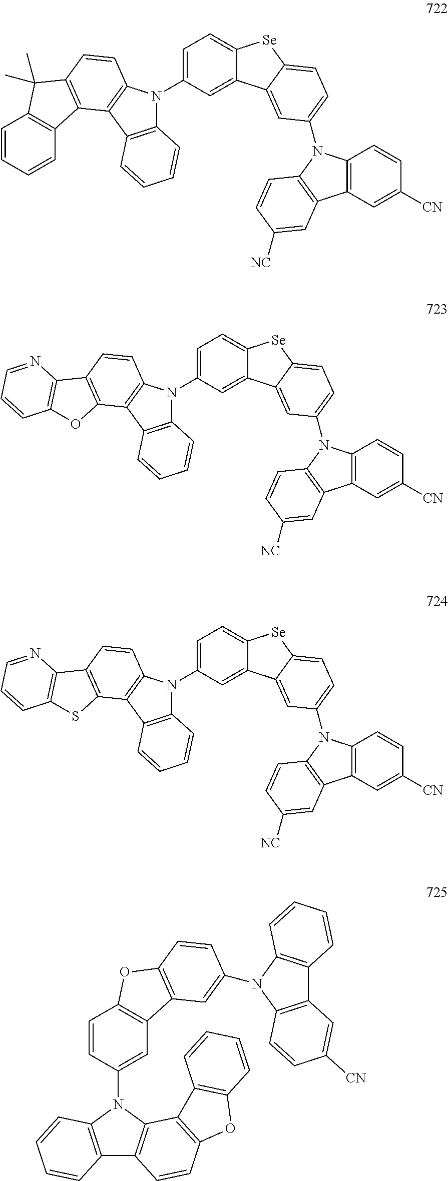 [SMILES: CC1(C)c2ccccc2-c2c1ccc1c2c2ccccc2n1-c1ccc2[se]c3ccc(-n4c5ccc(C#N)cc5c5cc(C#N)ccc54)cc3c2c1 ; N#Cc1ccc2c(c1)c1cc(C#N)ccc1n2-c1ccc2[se]c3ccc(-n4c5ccccc5c5c6oc7cccnc7c6ccc54)cc3c2c1 ; N#Cc1ccc2c(c1)c1cc(C#N)ccc1n2-c1ccc2[se]c3ccc(-n4c5ccccc5c5c6sc7cccnc7c6ccc54)cc3c2c1 ; N#Cc1ccc2c(c1)c1ccccc1n2-c1ccc2oc3ccc(-n4c5ccccc5c5ccc6oc7ccccc7c6c54)cc3c2c1]

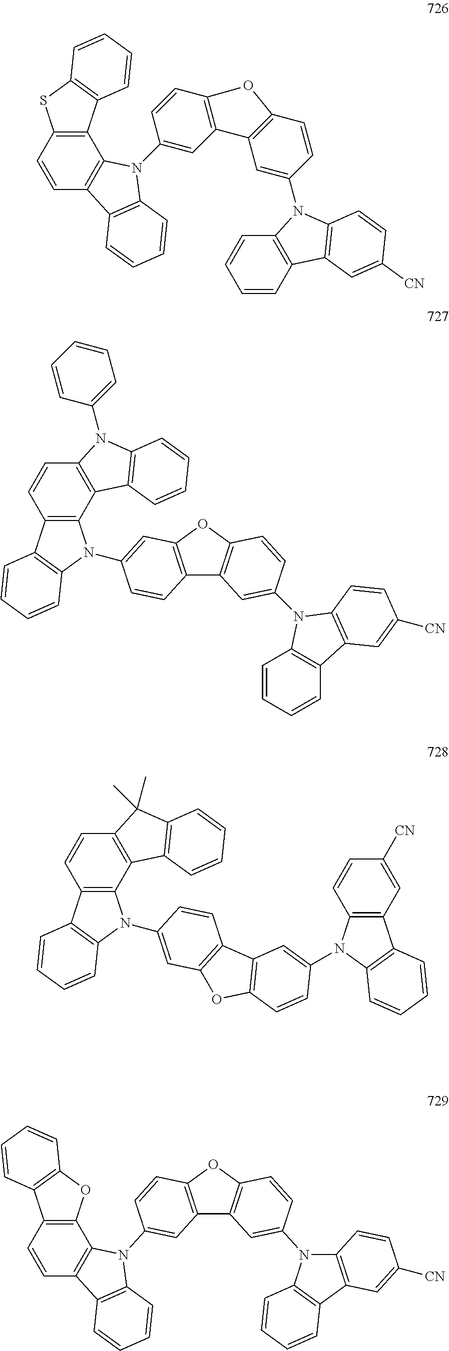 [SMILES: N#Cc1ccc2c(c1)c1ccccc1n2-c1ccc2oc3ccc(-n4c5ccccc5c5ccc6sc7ccccc7c6c54)cc3c2c1 ; N#Cc1ccc2c(c1)c1ccccc1n2-c1ccc2oc3cc(-n4c5ccccc5c5ccc6c(c7ccccc7n6-c6ccccc6)c54)ccc3c2c1 ; CC1(C)c2ccccc2-c2c1ccc1c3ccccc3n(-c3ccc4c(c3)oc3ccc(-n5c6ccccc6c6cc(C#N)ccc65)cc34)c21 ; N#Cc1ccc2c(c1)c1ccccc1n2-c1ccc2oc3ccc(-n4c5ccccc5c5ccc6c7ccccc7oc6c54)cc3c2c1]

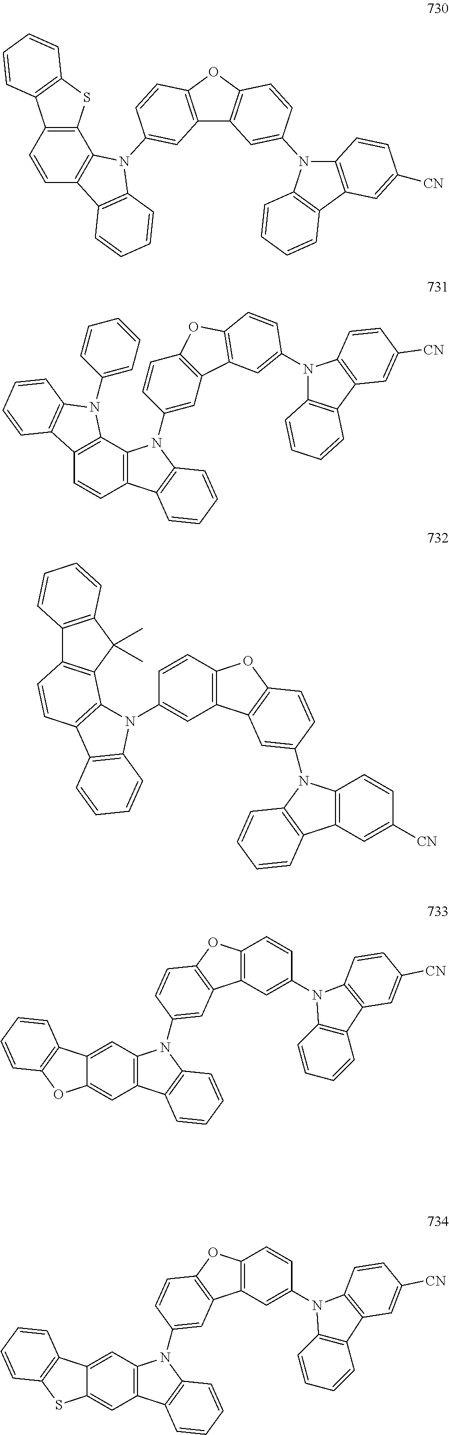 [SMILES: N#Cc1ccc2c(c1)c1ccccc1n2-c1ccc2oc3ccc(-n4c5ccccc5c5ccc6c7ccccc7sc6c54)cc3c2c1 ; N#Cc1ccc2c(c1)c1ccccc1n2-c1ccc2oc3ccc(-n4c5ccccc5c5ccc6c7ccccc7n(-c7ccccc7)c6c54)cc3c2c1 ; CC1(C)c2ccccc2-c2ccc3c4ccccc4n(-c4ccc5oc6ccc(-n7c8ccccc8c8cc(C#N)ccc87)cc6c5c4)c3c21 ; N#Cc1ccc2c(c1)c1ccccc1n2-c1ccc2oc3ccc(-n4c5ccccc5c5cc6oc7ccccc7c6cc54)cc3c2c1 ; N#Cc1ccc2c(c1)c1ccccc1n2-c1ccc2oc3ccc(-n4c5ccccc5c5cc6sc7ccccc7c6cc54)cc3c2c1]

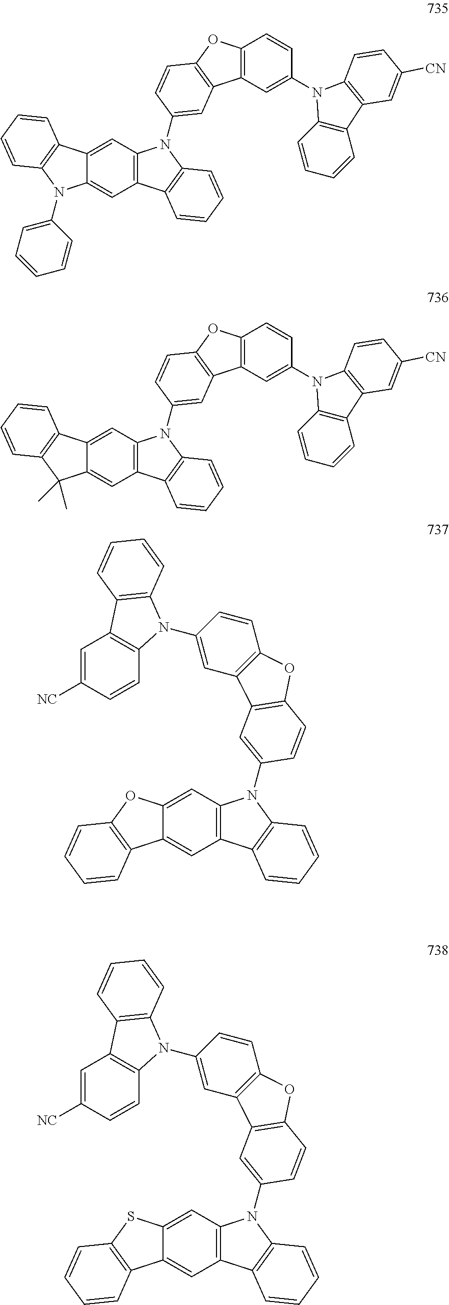 [SMILES: N#Cc1ccc2c(c1)c1ccccc1n2-c1ccc2oc3ccc(-n4c5ccccc5c5cc6c(cc54)c4ccccc4n6-c4ccccc4)cc3c2c1 ; CC1(C)c2ccccc2-c2cc3c(cc21)c1ccccc1n3-c1ccc2oc3ccc(-n4c5ccccc5c5cc(C#N)ccc54)cc3c2c1 ; N#Cc1ccc2c(c1)c1ccccc1n2-c1ccc2oc3ccc(-n4c5ccccc5c5cc6c(cc54)oc4ccccc46)cc3c2c1 ; N#Cc1ccc2c(c1)c1ccccc1n2-c1ccc2oc3ccc(-n4c5ccccc5c5cc6c(cc54)sc4ccccc46)cc3c2c1]

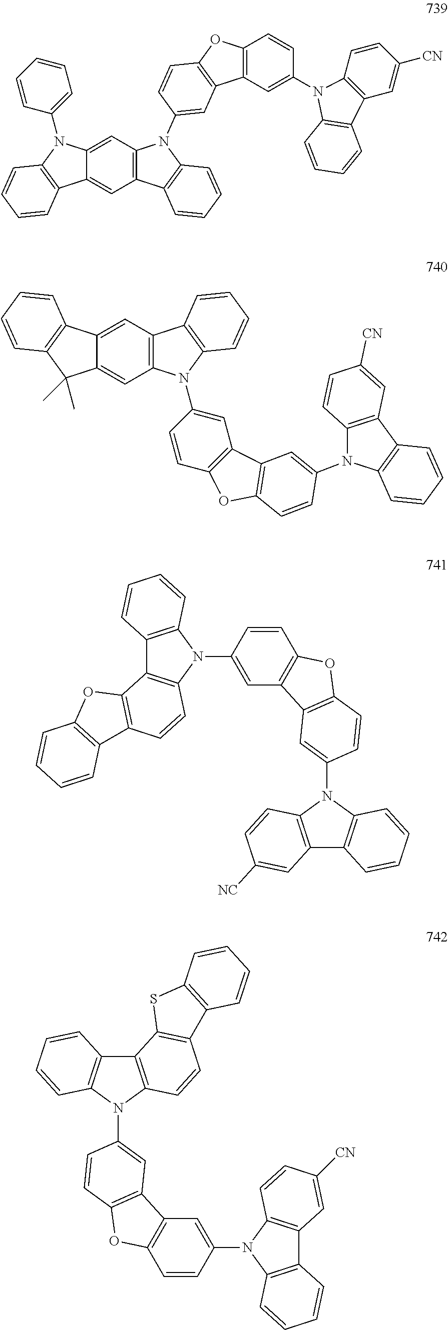 [SMILES: N#Cc1ccc2c(c1)c1ccccc1n2-c1ccc2oc3ccc(-n4c5ccccc5c5cc6c7ccccc7n(-c7ccccc7)c6cc54)cc3c2c1 ; CC1(C)c2ccccc2-c2cc3c4ccccc4n(-c4ccc5oc6ccc(-n7c8ccccc8c8cc(C#N)ccc87)cc6c5c4)c3cc21 ; N#Cc1ccc2c(c1)c1ccccc1n2-c1ccc2oc3ccc(-n4c5ccccc5c5c6oc7ccccc7c6ccc54)cc3c2c1 ; N#Cc1ccc2c(c1)c1ccccc1n2-c1ccc2oc3ccc(-n4c5ccccc5c5c6sc7ccccc7c6ccc54)cc3c2c1]

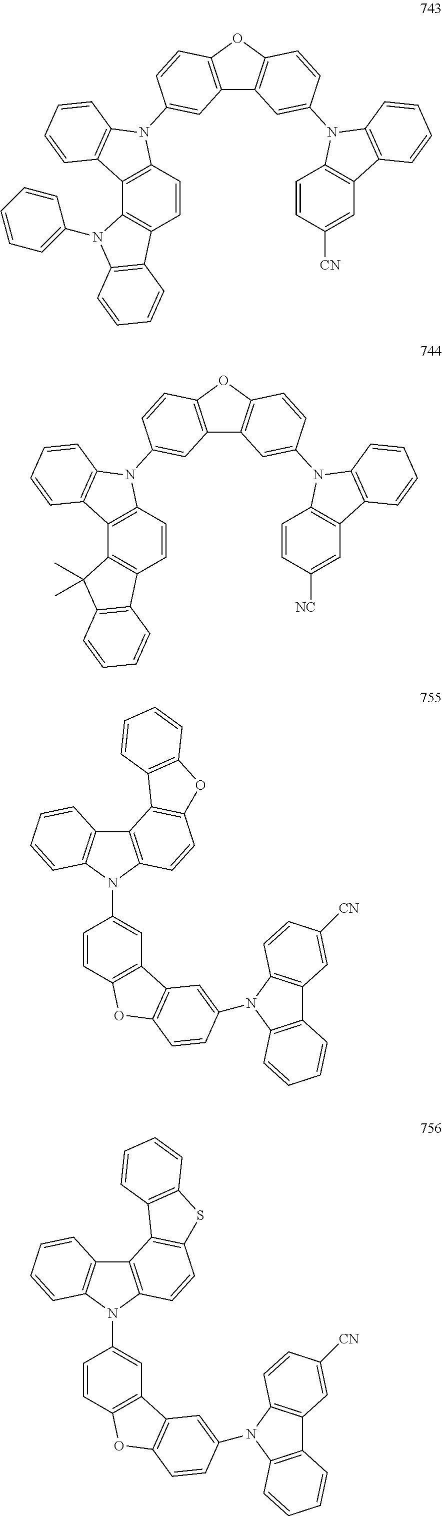 [SMILES: N#Cc1ccc2c(c1)c1ccccc1n2-c1ccc2oc3ccc(-n4c5ccccc5c5c4ccc4c6ccccc6n(-c6ccccc6)c45)cc3c2c1 ; CC1(C)c2ccccc2-c2ccc3c(c21)c1ccccc1n3-c1ccc2oc3ccc(-n4c5ccccc5c5cc(C#N)ccc54)cc3c2c1 ; N#Cc1ccc2c(c1)c1ccccc1n2-c1ccc2oc3ccc(-n4c5ccccc5c5c6c(ccc54)oc4ccccc46)cc3c2c1 ; N#Cc1ccc2c(c1)c1ccccc1n2-c1ccc2oc3ccc(-n4c5ccccc5c5c6c(ccc54)sc4ccccc46)cc3c2c1]

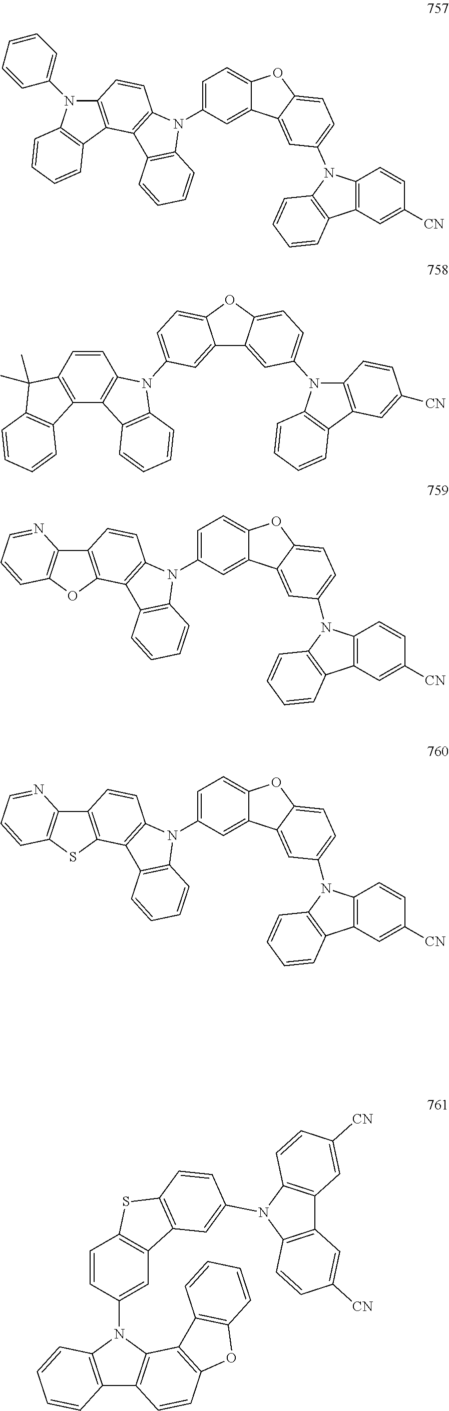 [SMILES: N#Cc1ccc2c(c1)c1ccccc1n2-c1ccc2oc3ccc(-n4c5ccccc5c5c6c7ccccc7n(-c7ccccc7)c6ccc54)cc3c2c1 ; CC1(C)c2ccccc2-c2c1ccc1c2c2ccccc2n1-c1ccc2oc3ccc(-n4c5ccccc5c5cc(C#N)ccc54)cc3c2c1 ; N#Cc1ccc2c(c1)c1ccccc1n2-c1ccc2oc3ccc(-n4c5ccccc5c5c6oc7cccnc7c6ccc54)cc3c2c1 ; N#Cc1ccc2c(c1)c1ccccc1n2-c1ccc2oc3ccc(-n4c5ccccc5c5c6sc7cccnc7c6ccc54)cc3c2c1 ; N#Cc1ccc2c(c1)c1cc(C#N)ccc1n2-c1ccc2sc3ccc(-n4c5ccccc5c5ccc6oc7ccccc7c6c54)cc3c2c1]

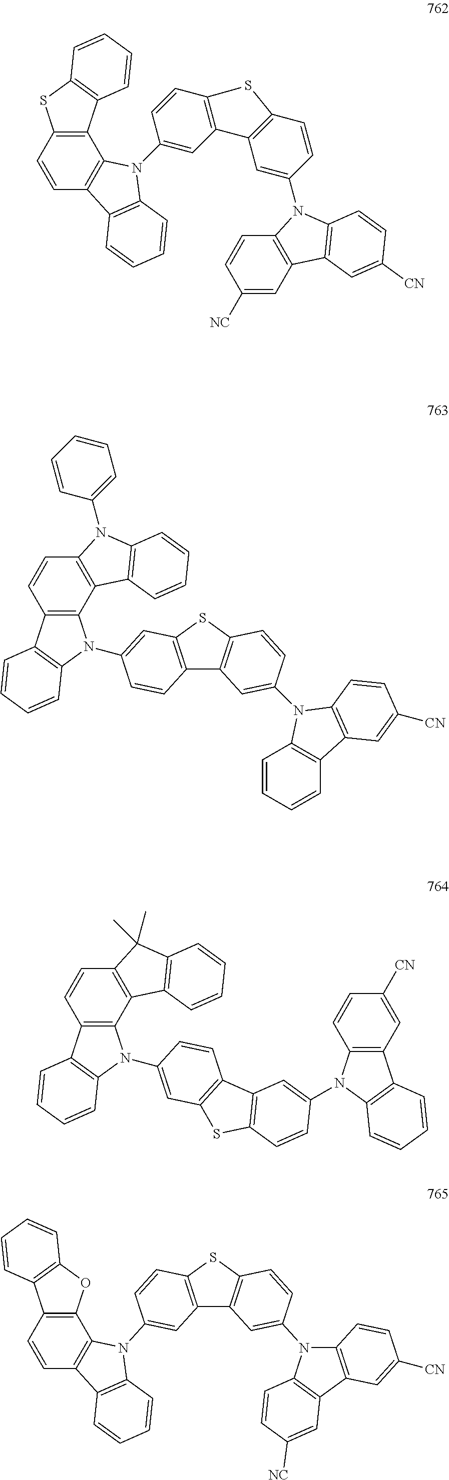 [SMILES: N#Cc1ccc2c(c1)c1cc(C#N)ccc1n2-c1ccc2sc3ccc(-n4c5ccccc5c5ccc6sc7ccccc7c6c54)cc3c2c1 ; N#Cc1ccc2c(c1)c1ccccc1n2-c1ccc2sc3cc(-n4c5ccccc5c5ccc6c(c7ccccc7n6-c6ccccc6)c54)ccc3c2c1 ; CC1(C)c2ccccc2-c2c1ccc1c3ccccc3n(-c3ccc4c(c3)sc3ccc(-n5c6ccccc6c6cc(C#N)ccc65)cc34)c21 ; N#Cc1ccc2c(c1)c1cc(C#N)ccc1n2-c1ccc2sc3ccc(-n4c5ccccc5c5ccc6c7ccccc7oc6c54)cc3c2c1]

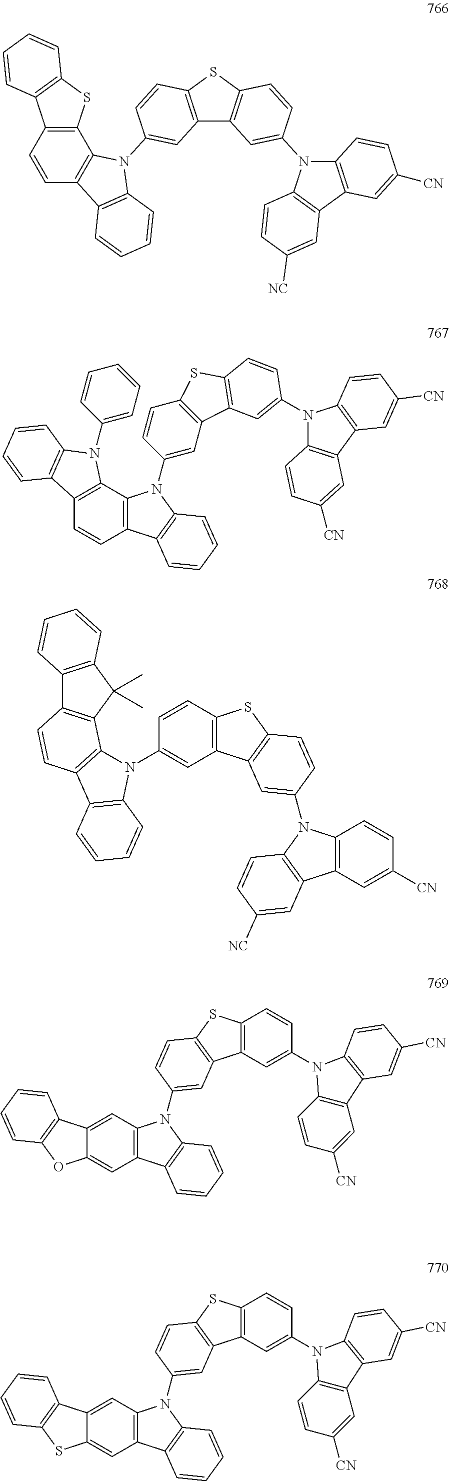 [SMILES: N#Cc1ccc2c(c1)c1cc(C#N)ccc1n2-c1ccc2sc3ccc(-n4c5ccccc5c5ccc6c7ccccc7sc6c54)cc3c2c1 ; N#Cc1ccc2c(c1)c1cc(C#N)ccc1n2-c1ccc2sc3ccc(-n4c5ccccc5c5ccc6c7ccccc7n(-c7ccccc7)c6c54)cc3c2c1 ; CC1(C)c2ccccc2-c2ccc3c4ccccc4n(-c4ccc5sc6ccc(-n7c8ccc(C#N)cc8c8cc(C#N)ccc87)cc6c5c4)c3c21 ; N#Cc1ccc2c(c1)c1cc(C#N)ccc1n2-c1ccc2sc3ccc(-n4c5ccccc5c5cc6oc7ccccc7c6cc54)cc3c2c1 ; N#Cc1ccc2c(c1)c1cc(C#N)ccc1n2-c1ccc2sc3ccc(-n4c5ccccc5c5cc6sc7ccccc7c6cc54)cc3c2c1]

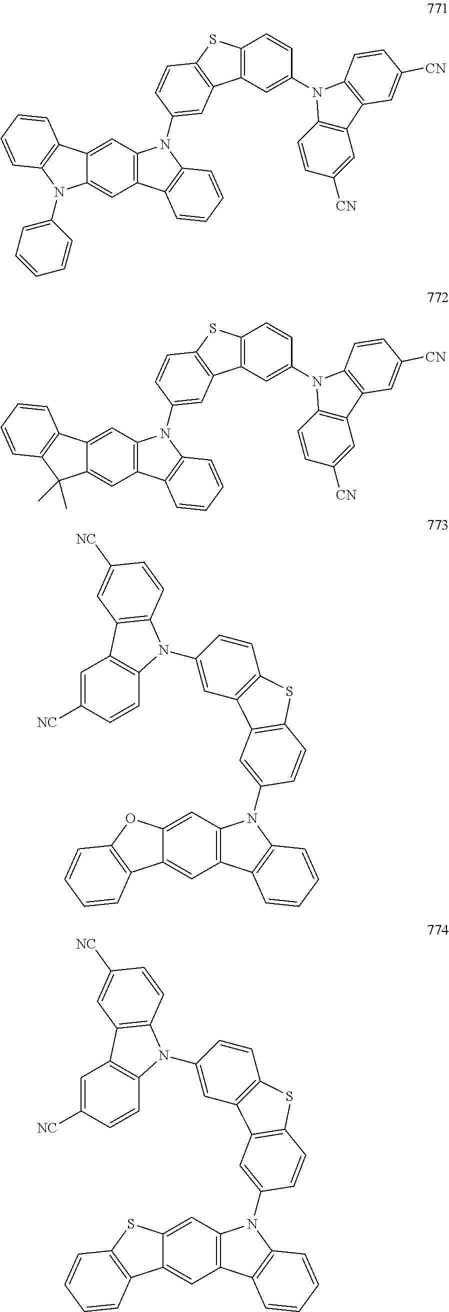

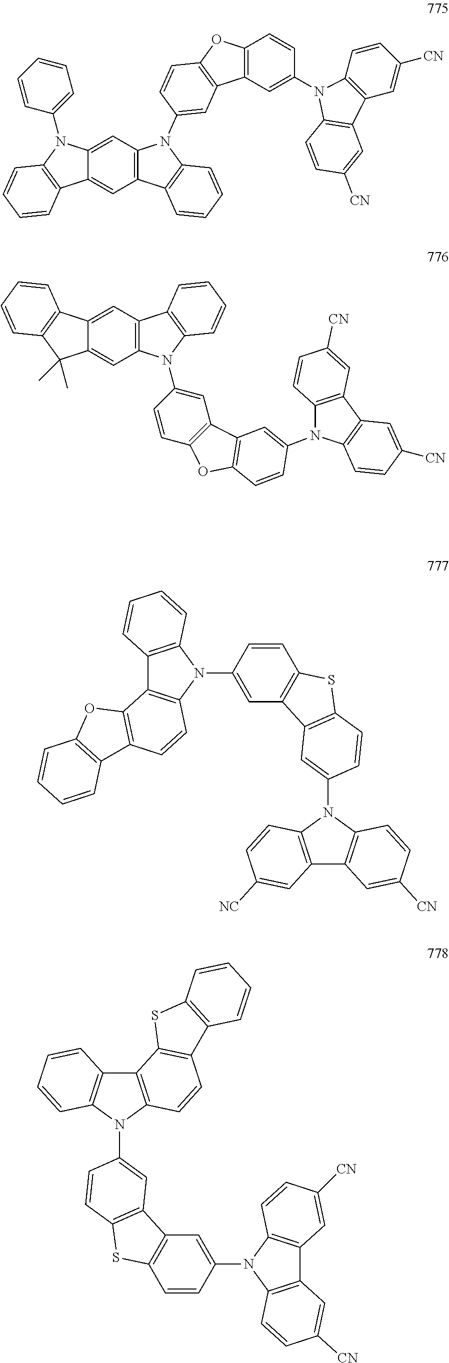 [SMILES: N#Cc1ccc2c(c1)c1cc(C#N)ccc1n2-c1ccc2oc3ccc(-n4c5ccccc5c5cc6c7ccccc7n(-c7ccccc7)c6cc54)cc3c2c1 ; CC1(C)c2ccccc2-c2cc3c4ccccc4n(-c4ccc5oc6ccc(-n7c8ccc(C#N)cc8c8cc(C#N)ccc87)cc6c5c4)c3cc21 ; N#Cc1ccc2c(c1)c1cc(C#N)ccc1n2-c1ccc2sc3ccc(-n4c5ccccc5c5c6oc7ccccc7c6ccc54)cc3c2c1 ; N#Cc1ccc2c(c1)c1cc(C#N)ccc1n2-c1ccc2sc3ccc(-n4c5ccccc5c5c6sc7ccccc7c6ccc54)cc3c2c1]

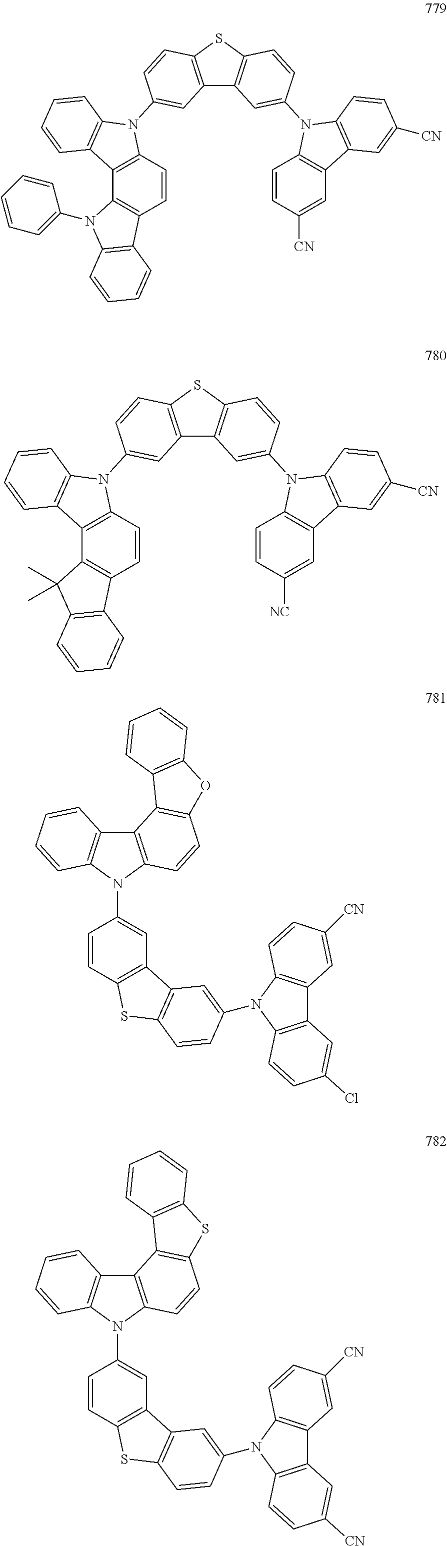 [SMILES: N#Cc1ccc2c(c1)c1cc(C#N)ccc1n2-c1ccc2sc3ccc(-n4c5ccccc5c5c4ccc4c6ccccc6n(-c6ccccc6)c45)cc3c2c1 ; CC1(C)c2ccccc2-c2ccc3c(c21)c1ccccc1n3-c1ccc2sc3ccc(-n4c5ccc(C#N)cc5c5cc(C#N)ccc54)cc3c2c1 ; N#Cc1ccc2c(c1)c1cc(Cl)ccc1n2-c1ccc2sc3ccc(-n4c5ccccc5c5c6c(ccc54)oc4ccccc46)cc3c2c1 ; N#Cc1ccc2c(c1)c1cc(C#N)ccc1n2-c1ccc2sc3ccc(-n4c5ccccc5c5c6c(ccc54)sc4ccccc46)cc3c2c1]

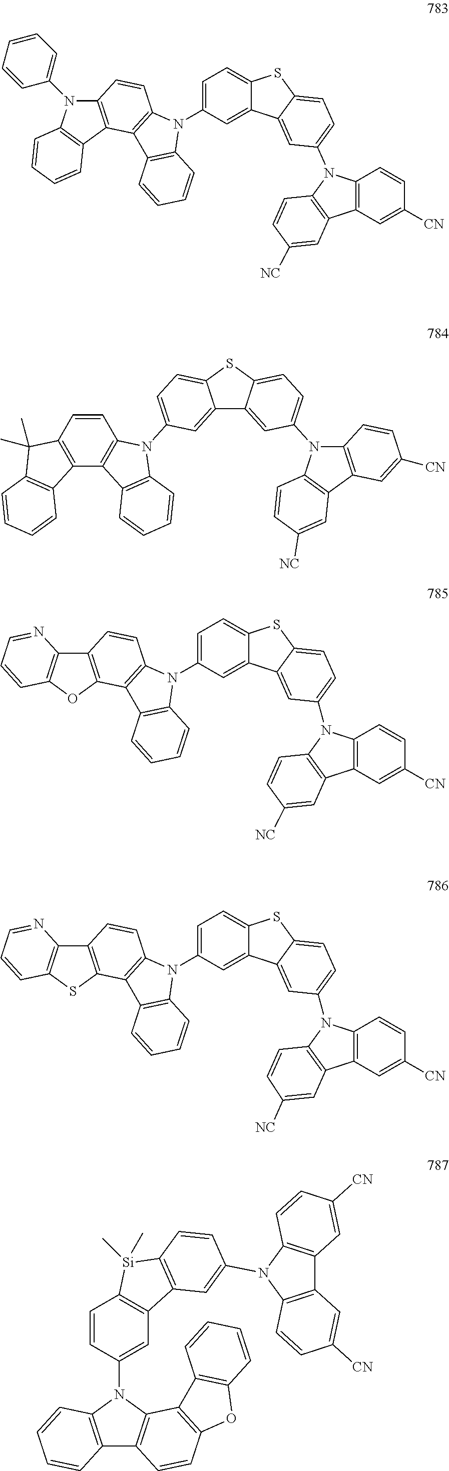 [SMILES: N#Cc1ccc2c(c1)c1cc(C#N)ccc1n2-c1ccc2sc3ccc(-n4c5ccccc5c5c6c7ccccc7n(-c7ccccc7)c6ccc54)cc3c2c1 ; CC1(C)c2ccccc2-c2c1ccc1c2c2ccccc2n1-c1ccc2sc3ccc(-n4c5ccc(C#N)cc5c5cc(C#N)ccc54)cc3c2c1 ; N#Cc1ccc2c(c1)c1cc(C#N)ccc1n2-c1ccc2sc3ccc(-n4c5ccccc5c5c6oc7cccnc7c6ccc54)cc3c2c1 ; N#Cc1ccc2c(c1)c1cc(C#N)ccc1n2-c1ccc2sc3ccc(-n4c5ccccc5c5c6sc7cccnc7c6ccc54)cc3c2c1 ; C[Si]1(C)c2ccc(-n3c4ccc(C#N)cc4c4cc(C#N)ccc43)cc2-c2cc(-n3c4ccccc4c4ccc5oc6ccccc6c5c43)ccc21]

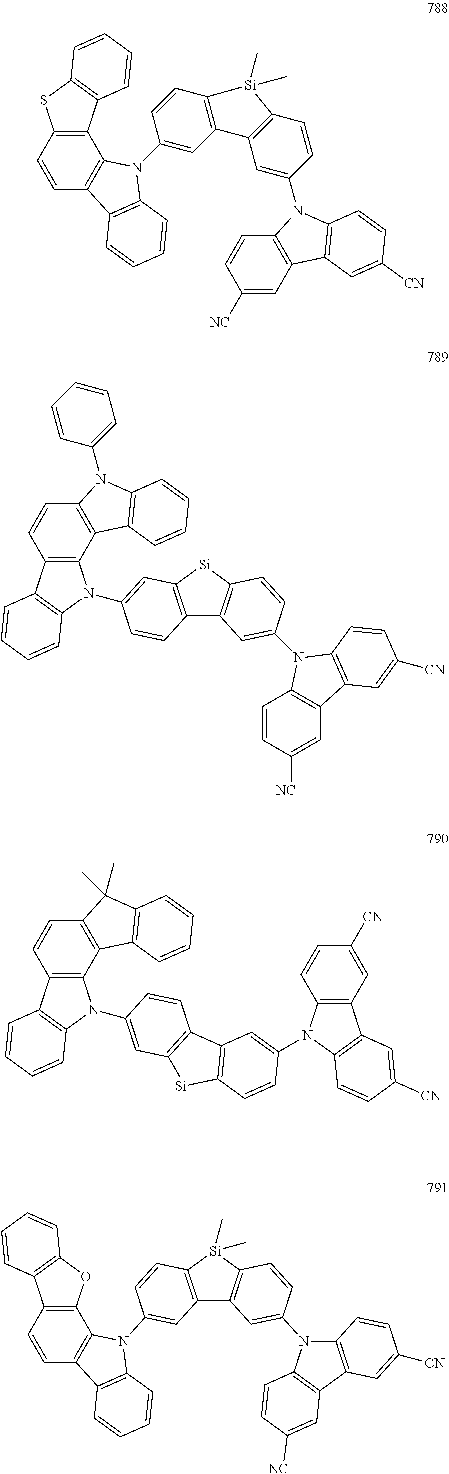

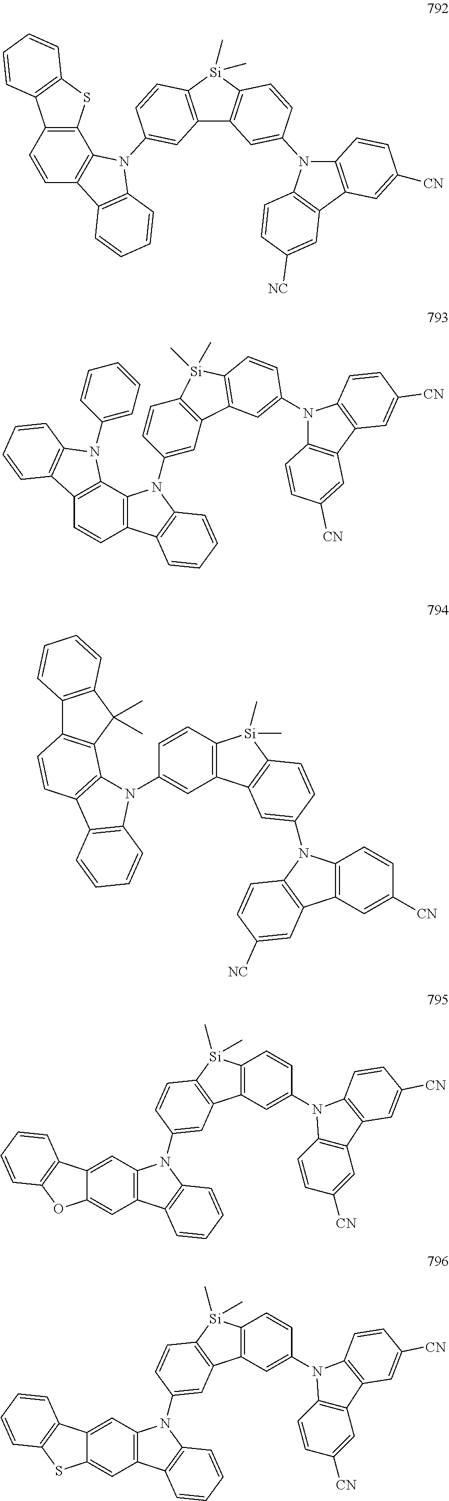 [SMILES: C[Si]1(C)c2ccc(-n3c4ccc(C#N)cc4c4cc(C#N)ccc43)cc2-c2cc(-n3c4ccccc4c4ccc5c6ccccc6sc5c43)ccc21 ; C[Si]1(C)c2ccc(-n3c4ccc(C#N)cc4c4cc(C#N)ccc43)cc2-c2cc(-n3c4ccccc4c4ccc5c6ccccc6n(-c6ccccc6)c5c43)ccc21 ; CC1(C)c2ccccc2-c2ccc3c4ccccc4n(-c4ccc5c(c4)-c4cc(-n6c7ccc(C#N)cc7c7cc(C#N)ccc76)ccc4[Si]5(C)C)c3c21 ; C[Si]1(C)c2ccc(-n3c4ccc(C#N)cc4c4cc(C#N)ccc43)cc2-c2cc(-n3c4ccccc4c4cc5oc6ccccc6c5cc43)ccc21 ; C[Si]1(C)c2ccc(-n3c4ccc(C#N)cc4c4cc(C#N)ccc43)cc2-c2cc(-n3c4ccccc4c4cc5sc6ccccc6c5cc43)ccc21]

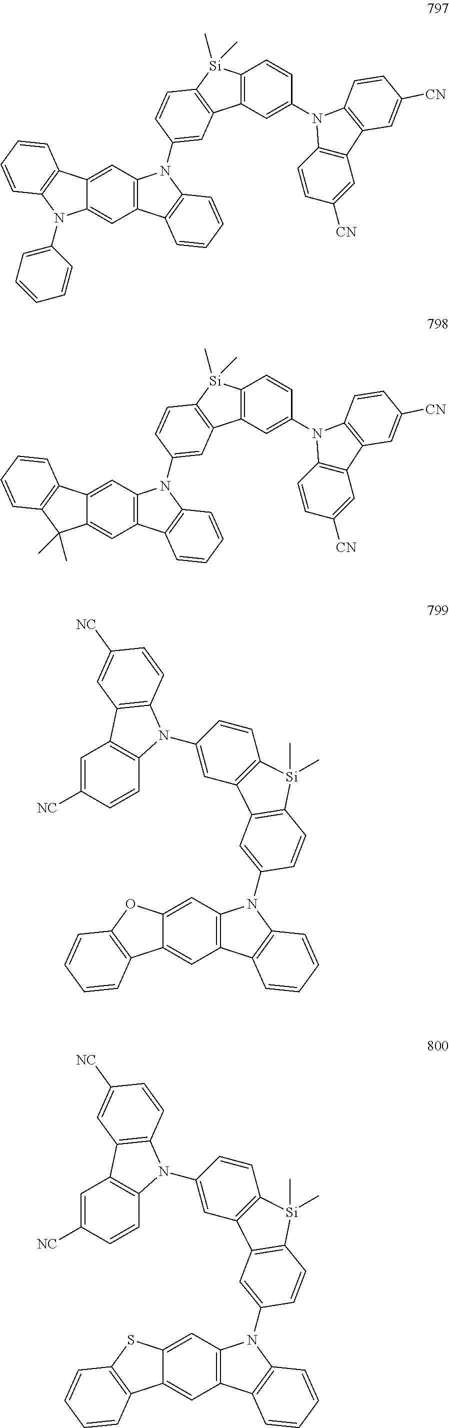 [SMILES: C[Si]1(C)c2ccc(-n3c4ccc(C#N)cc4c4cc(C#N)ccc43)cc2-c2cc(-n3c4ccccc4c4cc5c(cc43)c3ccccc3n5-c3ccccc3)ccc21 ; CC1(C)c2ccccc2-c2cc3c(cc21)c1ccccc1n3-c1ccc2c(c1)-c1cc(-n3c4ccc(C#N)cc4c4cc(C#N)ccc43)ccc1[Si]2(C)C ; C[Si]1(C)c2ccc(-n3c4ccc(C#N)cc4c4cc(C#N)ccc43)cc2-c2cc(-n3c4ccccc4c4cc5c(cc43)oc3ccccc35)ccc21 ; C[Si]1(C)c2ccc(-n3c4ccc(C#N)cc4c4cc(C#N)ccc43)cc2-c2cc(-n3c4ccccc4c4cc5c(cc43)sc3ccccc35)ccc21]

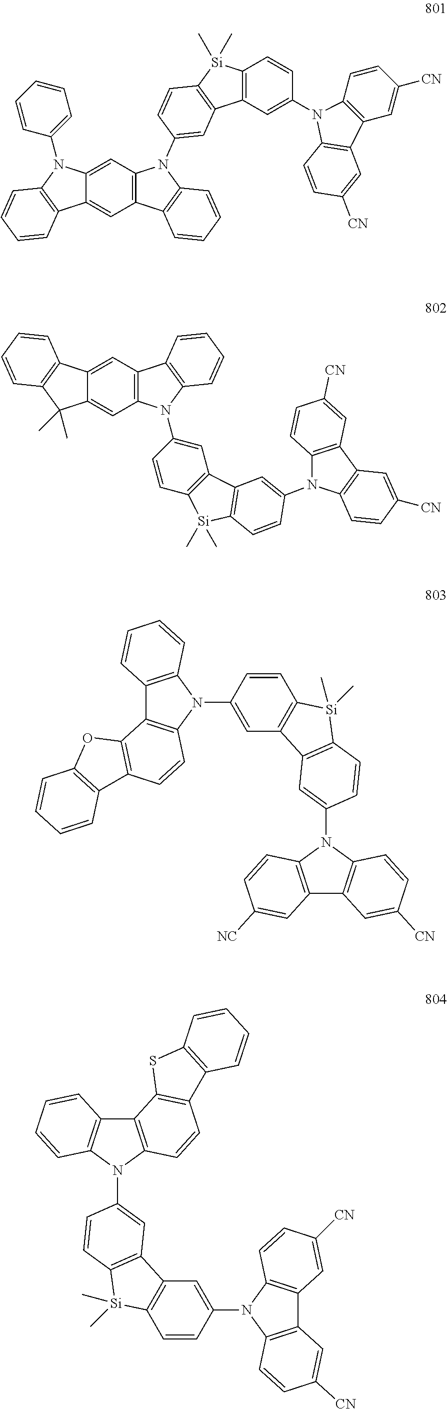 [SMILES: C[Si]1(C)c2ccc(-n3c4ccc(C#N)cc4c4cc(C#N)ccc43)cc2-c2cc(-n3c4ccccc4c4cc5c6ccccc6n(-c6ccccc6)c5cc43)ccc21 ; CC1(C)c2ccccc2-c2cc3c4ccccc4n(-c4ccc5c(c4)-c4cc(-n6c7ccc(C#N)cc7c7cc(C#N)ccc76)ccc4[Si]5(C)C)c3cc21 ; C[Si]1(C)c2ccc(-n3c4ccc(C#N)cc4c4cc(C#N)ccc43)cc2-c2cc(-n3c4ccccc4c4c5oc6ccccc6c5ccc43)ccc21 ; C[Si]1(C)c2ccc(-n3c4ccc(C#N)cc4c4cc(C#N)ccc43)cc2-c2cc(-n3c4ccccc4c4c5sc6ccccc6c5ccc43)ccc21]

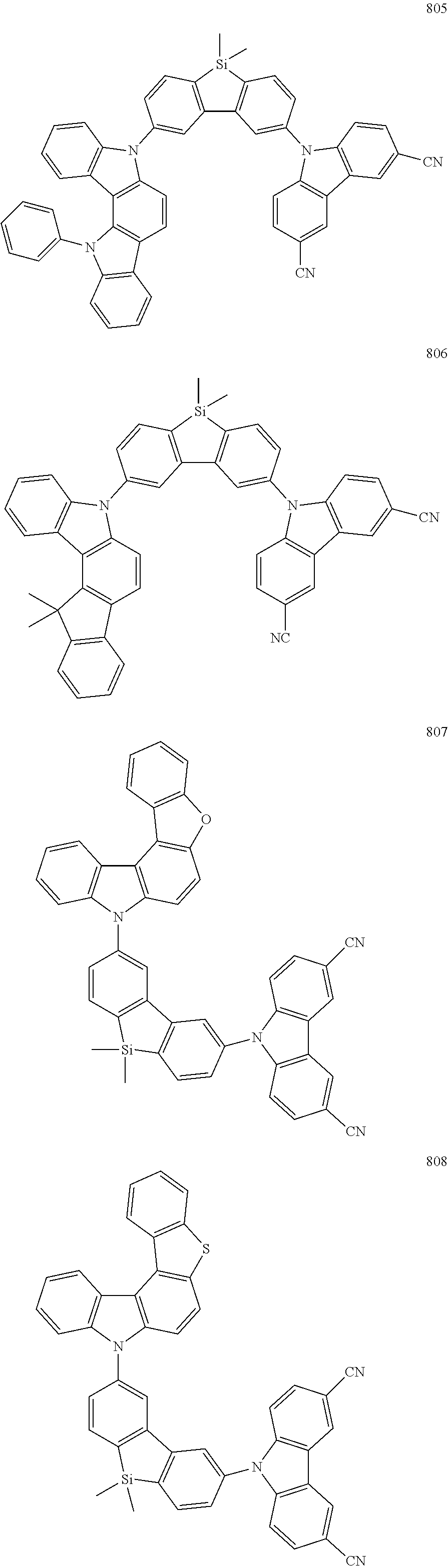 [SMILES: C[Si]1(C)c2ccc(-n3c4ccc(C#N)cc4c4cc(C#N)ccc43)cc2-c2cc(-n3c4ccccc4c4c3ccc3c5ccccc5n(-c5ccccc5)c34)ccc21 ; CC1(C)c2ccccc2-c2ccc3c(c21)c1ccccc1n3-c1ccc2c(c1)-c1cc(-n3c4ccc(C#N)cc4c4cc(C#N)ccc43)ccc1[Si]2(C)C ; C[Si]1(C)c2ccc(-n3c4ccc(C#N)cc4c4cc(C#N)ccc43)cc2-c2cc(-n3c4ccccc4c4c5c(ccc43)oc3ccccc35)ccc21 ; C[Si]1(C)c2ccc(-n3c4ccc(C#N)cc4c4cc(C#N)ccc43)cc2-c2cc(-n3c4ccccc4c4c5c(ccc43)sc3ccccc35)ccc21]

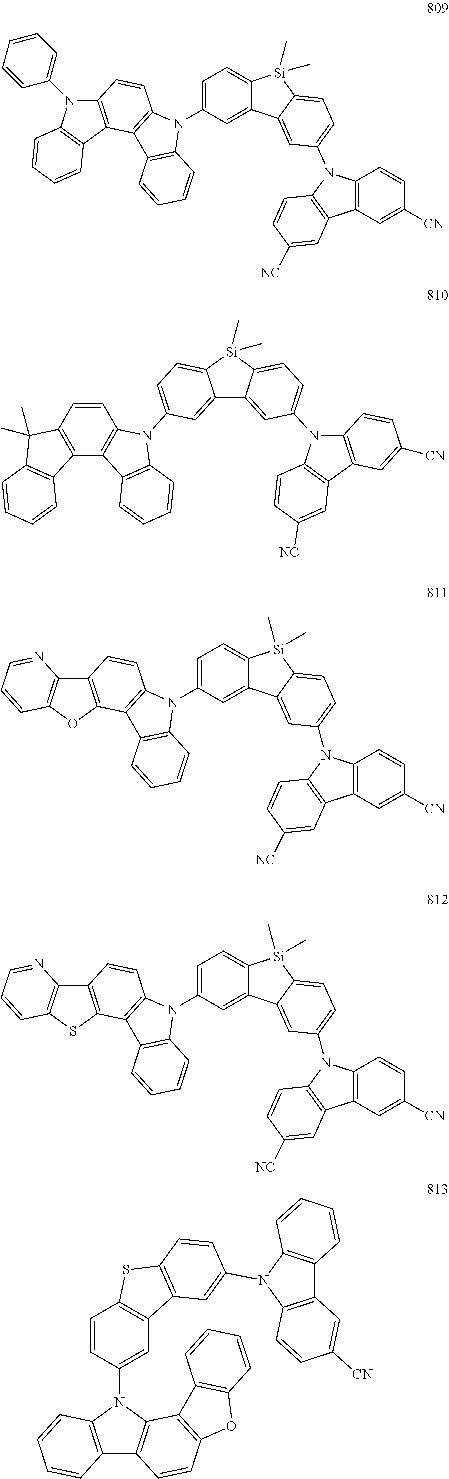 [SMILES: C[Si]1(C)c2ccc(-n3c4ccc(C#N)cc4c4cc(C#N)ccc43)cc2-c2cc(-n3c4ccccc4c4c5c6ccccc6n(-c6ccccc6)c5ccc43)ccc21 ; CC1(C)c2ccccc2-c2c1ccc1c2c2ccccc2n1-c1ccc2c(c1)-c1cc(-n3c4ccc(C#N)cc4c4cc(C#N)ccc43)ccc1[Si]2(C)C ; C[Si]1(C)c2ccc(-n3c4ccc(C#N)cc4c4cc(C#N)ccc43)cc2-c2cc(-n3c4ccccc4c4c5oc6cccnc6c5ccc43)ccc21 ; C[Si]1(C)c2ccc(-n3c4ccc(C#N)cc4c4cc(C#N)ccc43)cc2-c2cc(-n3c4ccccc4c4c5sc6cccnc6c5ccc43)ccc21 ; N#Cc1ccc2c(c1)c1ccccc1n2-c1ccc2sc3ccc(-n4c5ccccc5c5ccc6oc7ccccc7c6c54)cc3c2c1]

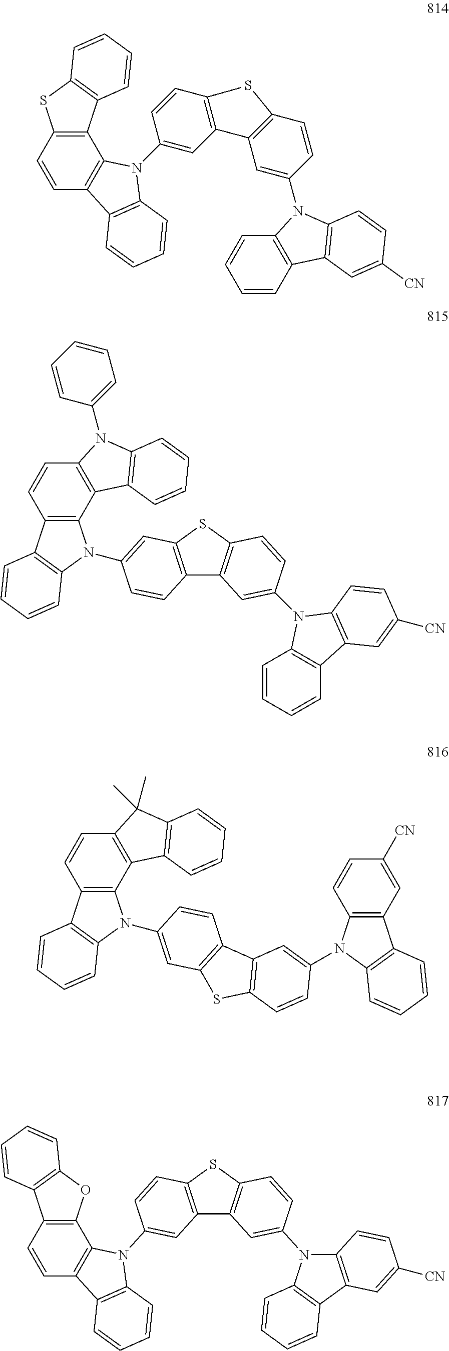 [SMILES: N#Cc1ccc2c(c1)c1ccccc1n2-c1ccc2sc3ccc(-n4c5ccccc5c5ccc6sc7ccccc7c6c54)cc3c2c1 ; N#Cc1ccc2c(c1)c1ccccc1n2-c1ccc2sc3cc(-n4c5ccccc5c5ccc6c(c7ccccc7n6-c6ccccc6)c54)ccc3c2c1 ; CC1(C)c2ccccc2-c2c1ccc1c3ccccc3n(-c3ccc4c(c3)sc3ccc(-n5c6ccccc6c6cc(C#N)ccc65)cc34)c21 ; N#Cc1ccc2c(c1)c1ccccc1n2-c1ccc2sc3ccc(-n4c5ccccc5c5ccc6c7ccccc7oc6c54)cc3c2c1]

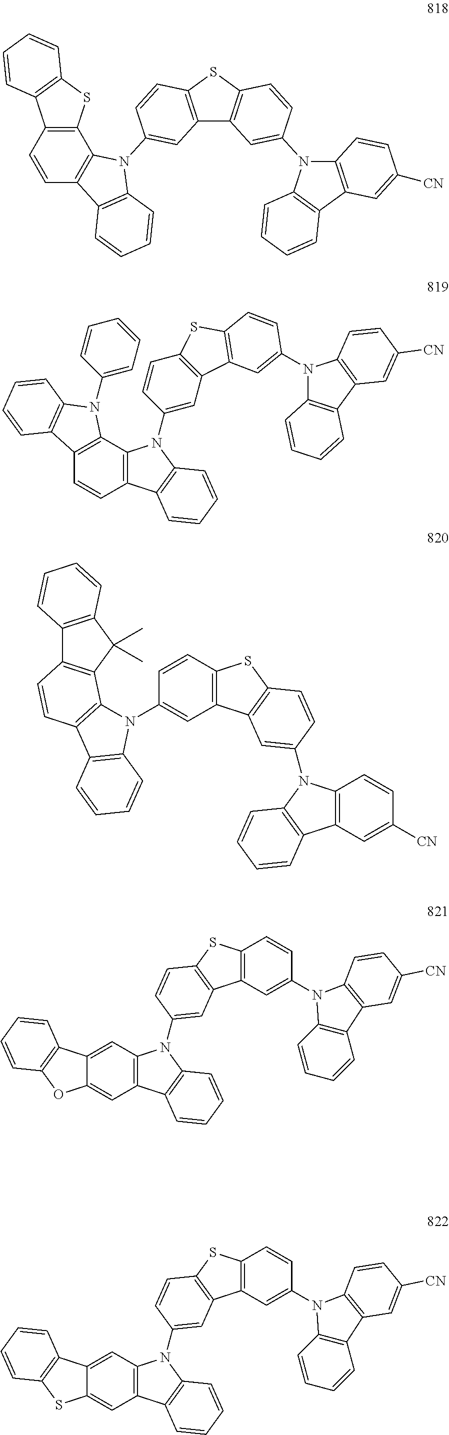 [SMILES: N#Cc1ccc2c(c1)c1ccccc1n2-c1ccc2sc3ccc(-n4c5ccccc5c5ccc6c7ccccc7sc6c54)cc3c2c1 ; N#Cc1ccc2c(c1)c1ccccc1n2-c1ccc2sc3ccc(-n4c5ccccc5c5ccc6c7ccccc7n(-c7ccccc7)c6c54)cc3c2c1 ; CC1(C)c2ccccc2-c2ccc3c4ccccc4n(-c4ccc5sc6ccc(-n7c8ccccc8c8cc(C#N)ccc87)cc6c5c4)c3c21 ; N#Cc1ccc2c(c1)c1ccccc1n2-c1ccc2sc3ccc(-n4c5ccccc5c5cc6oc7ccccc7c6cc54)cc3c2c1 ; N#Cc1ccc2c(c1)c1ccccc1n2-c1ccc2sc3ccc(-n4c5ccccc5c5cc6sc7ccccc7c6cc54)cc3c2c1]

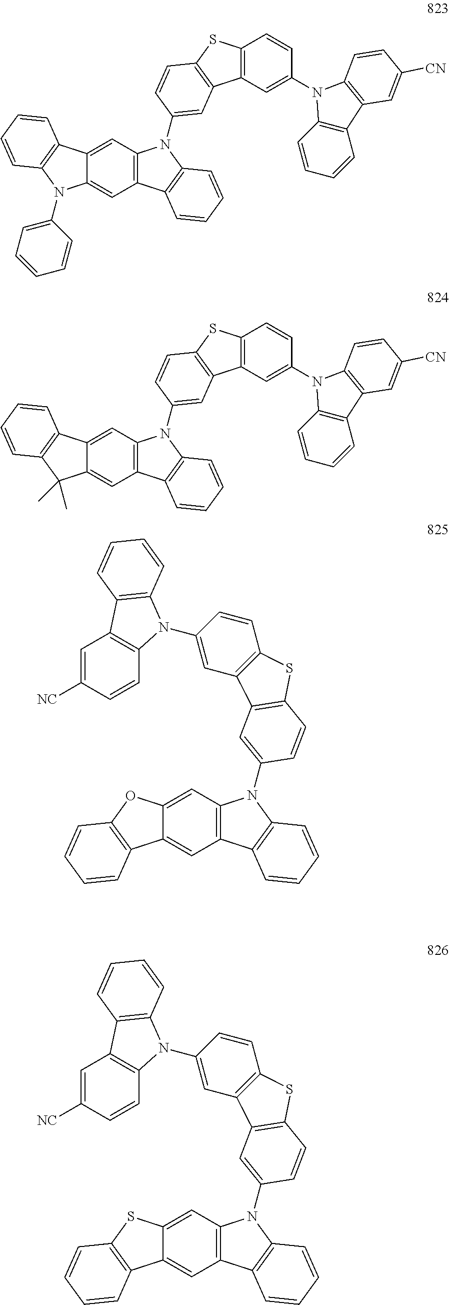 [SMILES: N#Cc1ccc2c(c1)c1ccccc1n2-c1ccc2sc3ccc(-n4c5ccccc5c5cc6c(cc54)c4ccccc4n6-c4ccccc4)cc3c2c1 ; CC1(C)c2ccccc2-c2cc3c(cc21)c1ccccc1n3-c1ccc2sc3ccc(-n4c5ccccc5c5cc(C#N)ccc54)cc3c2c1 ; N#Cc1ccc2c(c1)c1ccccc1n2-c1ccc2sc3ccc(-n4c5ccccc5c5cc6c(cc54)oc4ccccc46)cc3c2c1 ; N#Cc1ccc2c(c1)c1ccccc1n2-c1ccc2sc3ccc(-n4c5ccccc5c5cc6c(cc54)sc4ccccc46)cc3c2c1]

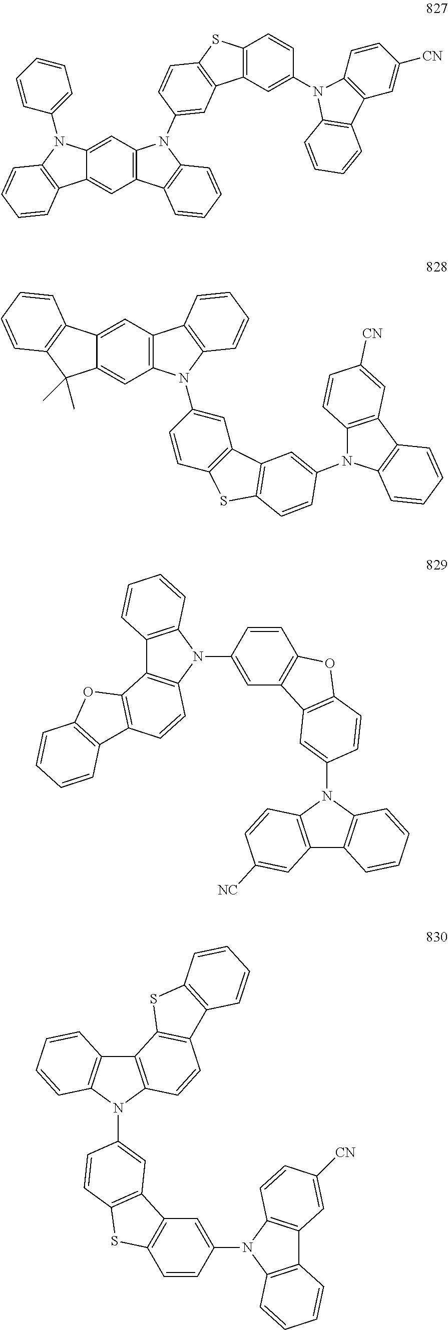 [SMILES: N#Cc1ccc2c(c1)c1ccccc1n2-c1ccc2sc3ccc(-n4c5ccccc5c5cc6c7ccccc7n(-c7ccccc7)c6cc54)cc3c2c1 ; CC1(C)c2ccccc2-c2cc3c4ccccc4n(-c4ccc5sc6ccc(-n7c8ccccc8c8cc(C#N)ccc87)cc6c5c4)c3cc21 ; N#Cc1ccc2c(c1)c1ccccc1n2-c1ccc2sc3ccc(-n4c5ccccc5c5c6oc7ccccc7c6ccc54)cc3c2c1 ; N#Cc1ccc2c(c1)c1ccccc1n2-c1ccc2sc3ccc(-n4c5ccccc5c5c6sc7ccccc7c6ccc54)cc3c2c1]

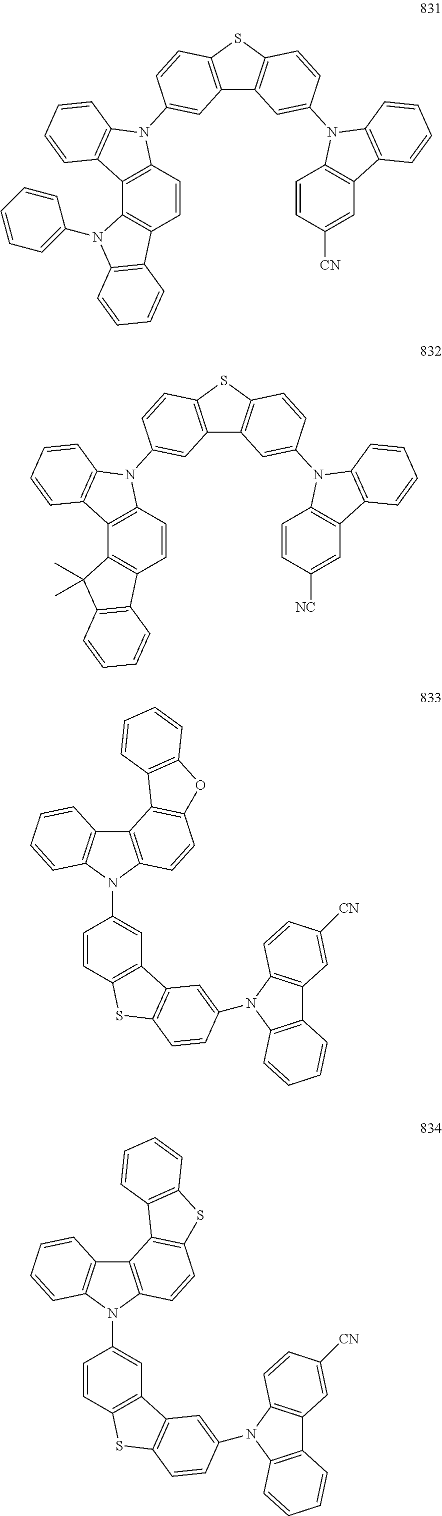 [SMILES: N#Cc1ccc2c(c1)c1ccccc1n2-c1ccc2sc3ccc(-n4c5ccccc5c5c4ccc4c6ccccc6n(-c6ccccc6)c45)cc3c2c1 ; CC1(C)c2ccccc2-c2ccc3c(c21)c1ccccc1n3-c1ccc2sc3ccc(-n4c5ccccc5c5cc(C#N)ccc54)cc3c2c1 ; N#Cc1ccc2c(c1)c1ccccc1n2-c1ccc2sc3ccc(-n4c5ccccc5c5c6c(ccc54)oc4ccccc46)cc3c2c1 ; N#Cc1ccc2c(c1)c1ccccc1n2-c1ccc2sc3ccc(-n4c5ccccc5c5c6c(ccc54)sc4ccccc46)cc3c2c1]

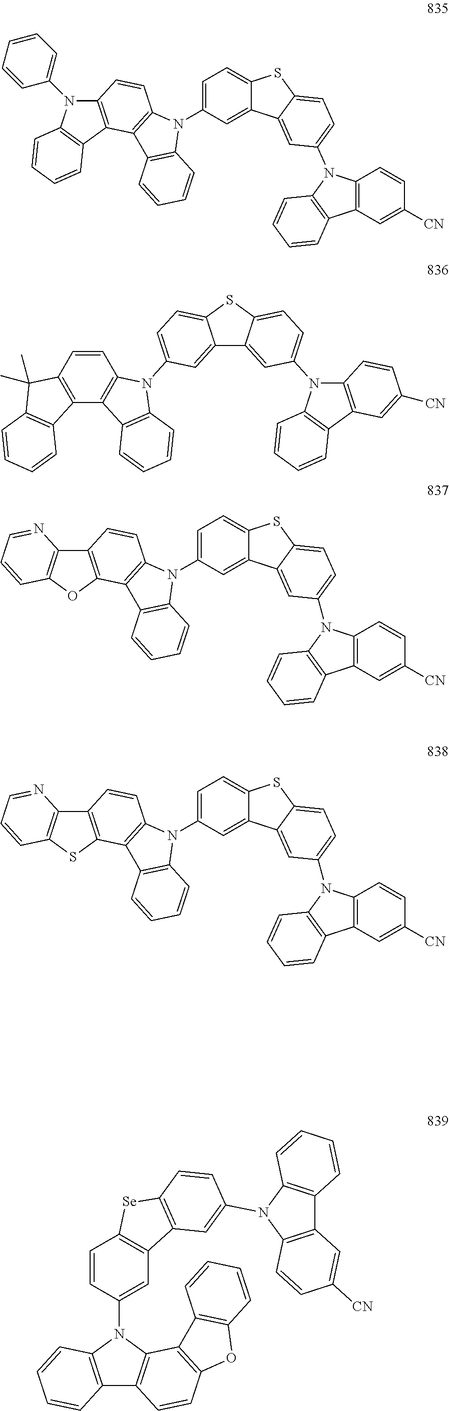 [SMILES: N#Cc1ccc2c(c1)c1ccccc1n2-c1ccc2sc3ccc(-n4c5ccccc5c5c6c7ccccc7n(-c7ccccc7)c6ccc54)cc3c2c1 ; CC1(C)c2ccccc2-c2c1ccc1c2c2ccccc2n1-c1ccc2sc3ccc(-n4c5ccccc5c5cc(C#N)ccc54)cc3c2c1 ; N#Cc1ccc2c(c1)c1ccccc1n2-c1ccc2sc3ccc(-n4c5ccccc5c5c6oc7cccnc7c6ccc54)cc3c2c1 ; N#Cc1ccc2c(c1)c1ccccc1n2-c1ccc2sc3ccc(-n4c5ccccc5c5c6sc7cccnc7c6ccc54)cc3c2c1 ; N#Cc1ccc2c(c1)c1ccccc1n2-c1ccc2[se]c3ccc(-n4c5ccccc5c5ccc6oc7ccccc7c6c54)cc3c2c1]

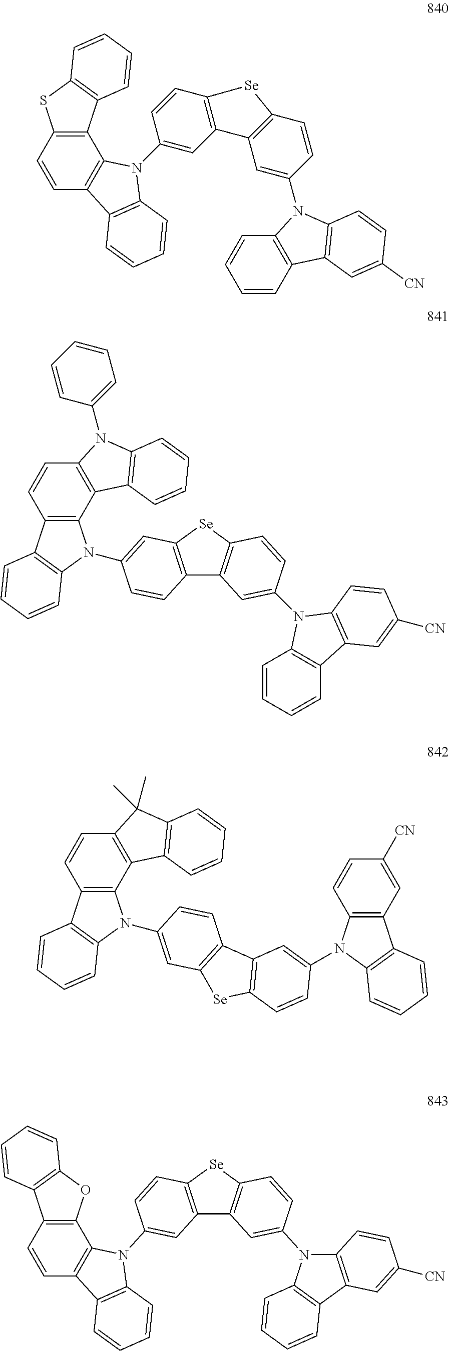 [SMILES: N#Cc1ccc2c(c1)c1ccccc1n2-c1ccc2[se]c3ccc(-n4c5ccccc5c5ccc6sc7ccccc7c6c54)cc3c2c1 ; N#Cc1ccc2c(c1)c1ccccc1n2-c1ccc2[se]c3cc(-n4c5ccccc5c5ccc6c(c7ccccc7n6-c6ccccc6)c54)ccc3c2c1 ; CC1(C)c2ccccc2-c2c1ccc1c3ccccc3n(-c3ccc4c(c3)[se]c3ccc(-n5c6ccccc6c6cc(C#N)ccc65)cc34)c21 ; N#Cc1ccc2c(c1)c1ccccc1n2-c1ccc2[se]c3ccc(-n4c5ccccc5c5ccc6c7ccccc7oc6c54)cc3c2c1]

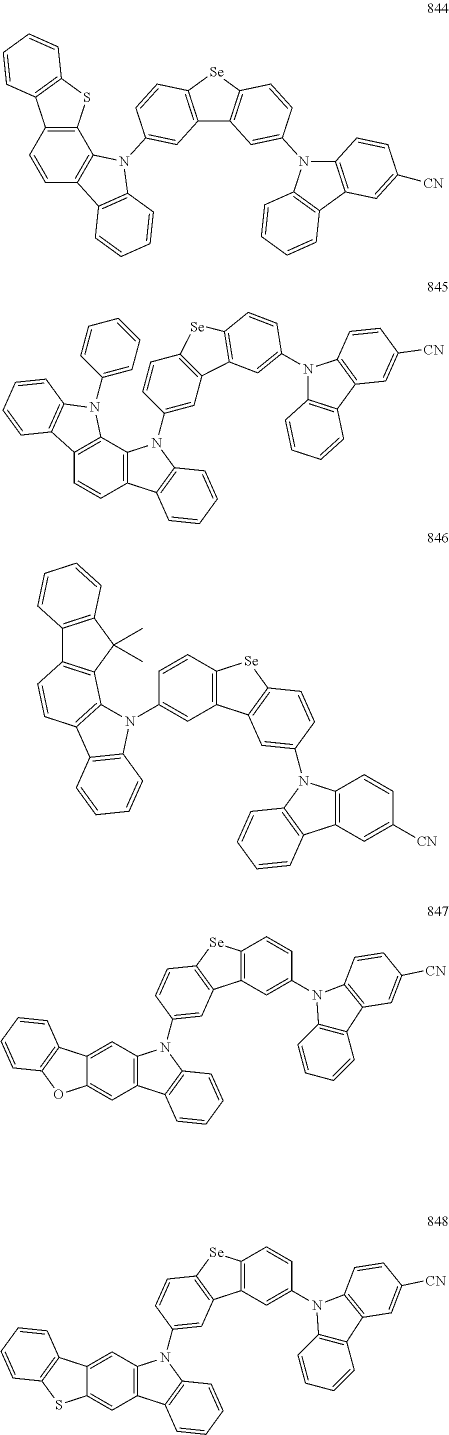 [SMILES: N#Cc1ccc2c(c1)c1ccccc1n2-c1ccc2[se]c3ccc(-n4c5ccccc5c5ccc6c7ccccc7sc6c54)cc3c2c1 ; N#Cc1ccc2c(c1)c1ccccc1n2-c1ccc2[se]c3ccc(-n4c5ccccc5c5ccc6c7ccccc7n(-c7ccccc7)c6c54)cc3c2c1 ; CC1(C)c2ccccc2-c2ccc3c4ccccc4n(-c4ccc5[se]c6ccc(-n7c8ccccc8c8cc(C#N)ccc87)cc6c5c4)c3c21 ; N#Cc1ccc2c(c1)c1ccccc1n2-c1ccc2[se]c3ccc(-n4c5ccccc5c5cc6oc7ccccc7c6cc54)cc3c2c1 ; N#Cc1ccc2c(c1)c1ccccc1n2-c1ccc2[se]c3ccc(-n4c5ccccc5c5cc6sc7ccccc7c6cc54)cc3c2c1]

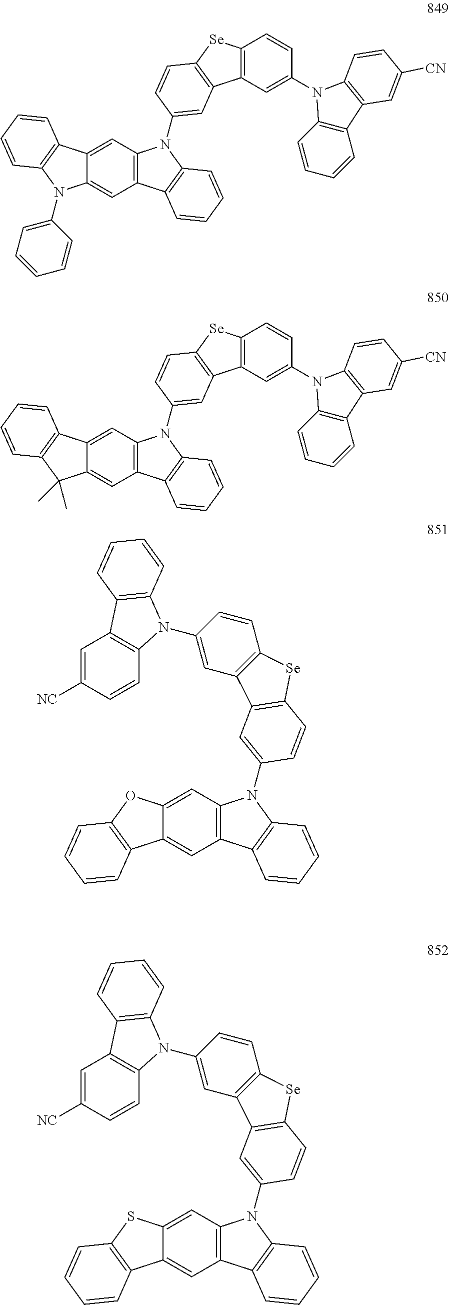 [SMILES: N#Cc1ccc2c(c1)c1ccccc1n2-c1ccc2[se]c3ccc(-n4c5ccccc5c5cc6c(cc54)c4ccccc4n6-c4ccccc4)cc3c2c1 ; CC1(C)c2ccccc2-c2cc3c(cc21)c1ccccc1n3-c1ccc2[se]c3ccc(-n4c5ccccc5c5cc(C#N)ccc54)cc3c2c1 ; N#Cc1ccc2c(c1)c1ccccc1n2-c1ccc2[se]c3ccc(-n4c5ccccc5c5cc6c(cc54)oc4ccccc46)cc3c2c1 ; N#Cc1ccc2c(c1)c1ccccc1n2-c1ccc2[se]c3ccc(-n4c5ccccc5c5cc6c(cc54)sc4ccccc46)cc3c2c1]

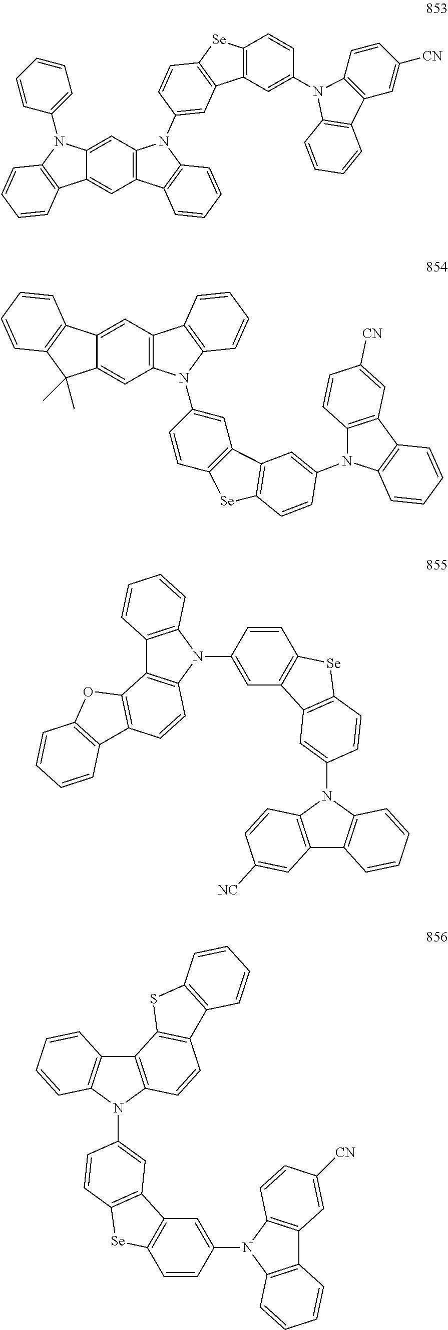 [SMILES: N#Cc1ccc2c(c1)c1ccccc1n2-c1ccc2[se]c3ccc(-n4c5ccccc5c5cc6c7ccccc7n(-c7ccccc7)c6cc54)cc3c2c1 ; CC1(C)c2ccccc2-c2cc3c4ccccc4n(-c4ccc5[se]c6ccc(-n7c8ccccc8c8cc(C#N)ccc87)cc6c5c4)c3cc21 ; N#Cc1ccc2c(c1)c1ccccc1n2-c1ccc2[se]c3ccc(-n4c5ccccc5c5c6oc7ccccc7c6ccc54)cc3c2c1 ; N#Cc1ccc2c(c1)c1ccccc1n2-c1ccc2[se]c3ccc(-n4c5ccccc5c5c6sc7ccccc7c6ccc54)cc3c2c1]

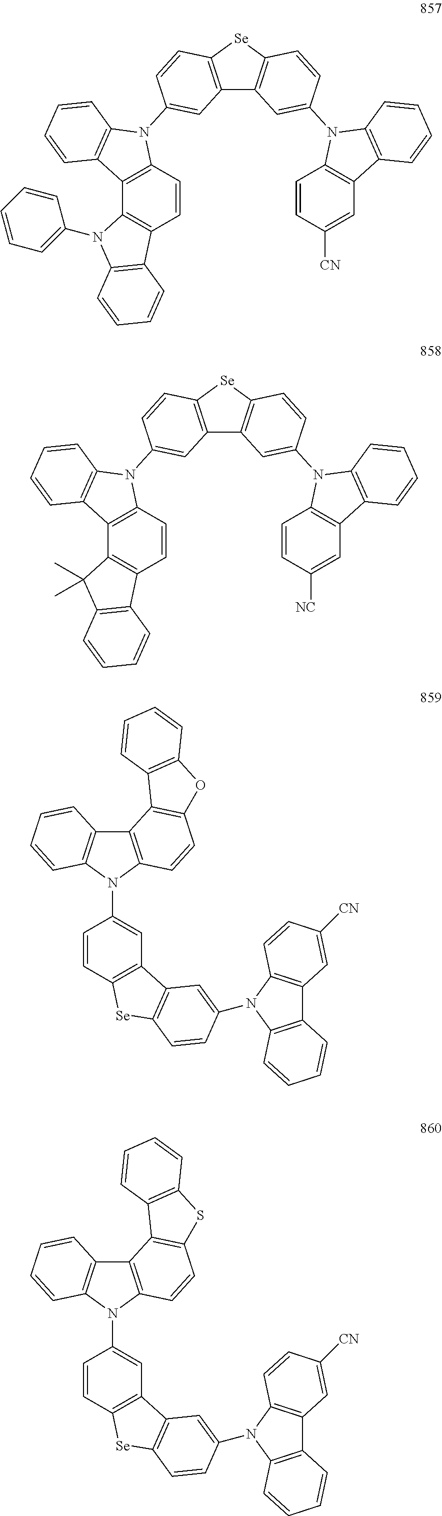 [SMILES: N#Cc1ccc2c(c1)c1ccccc1n2-c1ccc2[se]c3ccc(-n4c5ccccc5c5c4ccc4c6ccccc6n(-c6ccccc6)c45)cc3c2c1 ; CC1(C)c2ccccc2-c2ccc3c(c21)c1ccccc1n3-c1ccc2[se]c3ccc(-n4c5ccccc5c5cc(C#N)ccc54)cc3c2c1 ; N#Cc1ccc2c(c1)c1ccccc1n2-c1ccc2[se]c3ccc(-n4c5ccccc5c5c6c(ccc54)oc4ccccc46)cc3c2c1 ; N#Cc1ccc2c(c1)c1ccccc1n2-c1ccc2[se]c3ccc(-n4c5ccccc5c5c6c(ccc54)sc4ccccc46)cc3c2c1]

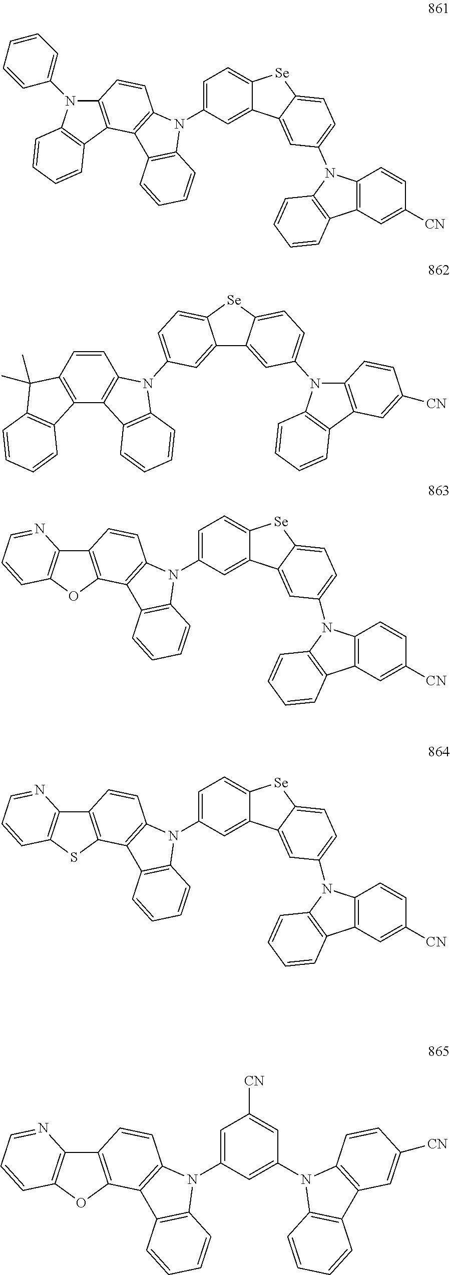 [SMILES: N#Cc1ccc2c(c1)c1ccccc1n2-c1ccc2[se]c3ccc(-n4c5ccccc5c5c6c7ccccc7n(-c7ccccc7)c6ccc54)cc3c2c1 ; CC1(C)c2ccccc2-c2c1ccc1c2c2ccccc2n1-c1ccc2[se]c3ccc(-n4c5ccccc5c5cc(C#N)ccc54)cc3c2c1 ; N#Cc1ccc2c(c1)c1ccccc1n2-c1ccc2[se]c3ccc(-n4c5ccccc5c5c6oc7cccnc7c6ccc54)cc3c2c1 ; N#Cc1ccc2c(c1)c1ccccc1n2-c1ccc2[se]c3ccc(-n4c5ccccc5c5c6sc7cccnc7c6ccc54)cc3c2c1 ; N#Cc1cc(-n2c3ccccc3c3cc(C#N)ccc32)cc(-n2c3ccccc3c3c4oc5cccnc5c4ccc32)c1]

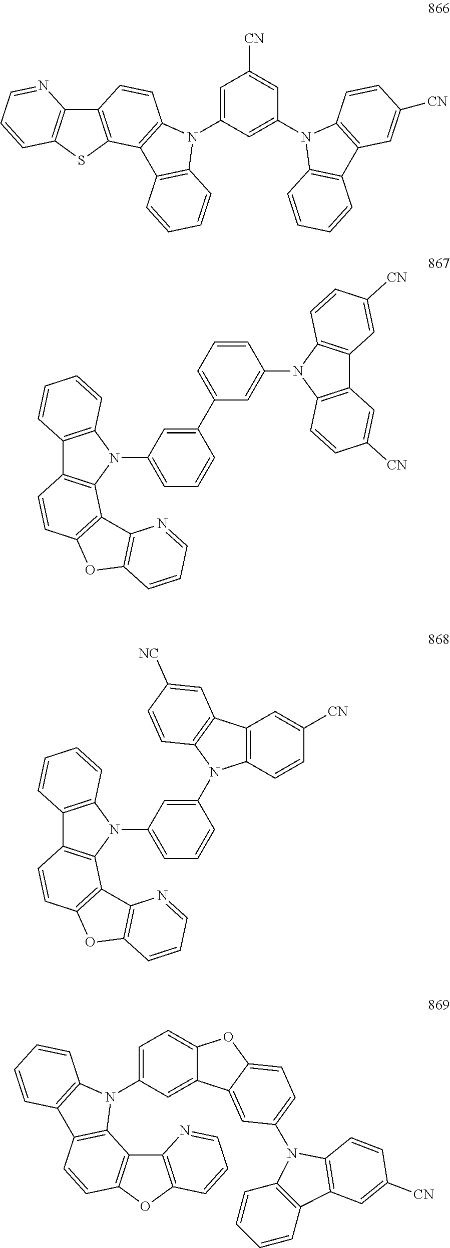 [SMILES: N#Cc1cc(-n2c3ccccc3c3cc(C#N)ccc32)cc(-n2c3ccccc3c3c4sc5cccnc5c4ccc32)c1 ; N#Cc1ccc2c(c1)c1cc(C#N)ccc1n2-c1cccc(-c2cccc(-n3c4ccccc4c4ccc5oc6cccnc6c5c43)c2)c1 ; N#Cc1ccc2c(c1)c1cc(C#N)ccc1n2-c1cccc(-n2c3ccccc3c3ccc4oc5cccnc5c4c32)c1 ; N#Cc1ccc2c(c1)c1ccccc1n2-c1ccc2oc3ccc(-n4c5ccccc5c5ccc6oc7cccnc7c6c54)cc3c2c1]

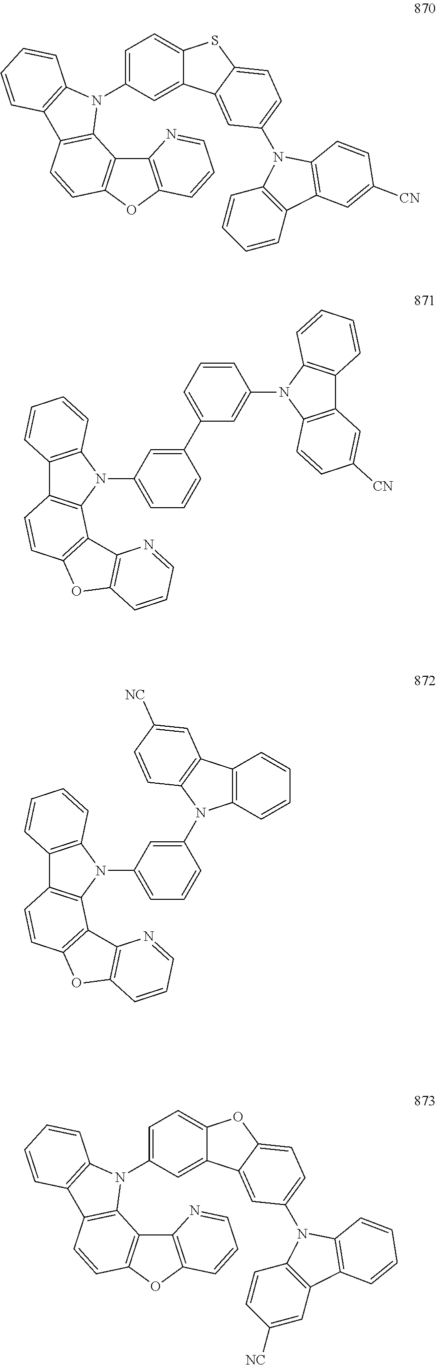 [SMILES: N#Cc1ccc2c(c1)c1ccccc1n2-c1ccc2sc3ccc(-n4c5ccccc5c5ccc6oc7cccnc7c6c54)cc3c2c1 ; N#Cc1ccc2c(c1)c1ccccc1n2-c1cccc(-c2cccc(-n3c4ccccc4c4ccc5oc6cccnc6c5c43)c2)c1 ; N#Cc1ccc2c(c1)c1ccccc1n2-c1cccc(-n2c3ccccc3c3ccc4oc5cccnc5c4c32)c1 ; N#Cc1ccc2c(c1)c1ccccc1n2-c1ccc2oc3ccc(-n4c5ccccc5c5ccc6oc7cccnc7c6c54)cc3c2c1]

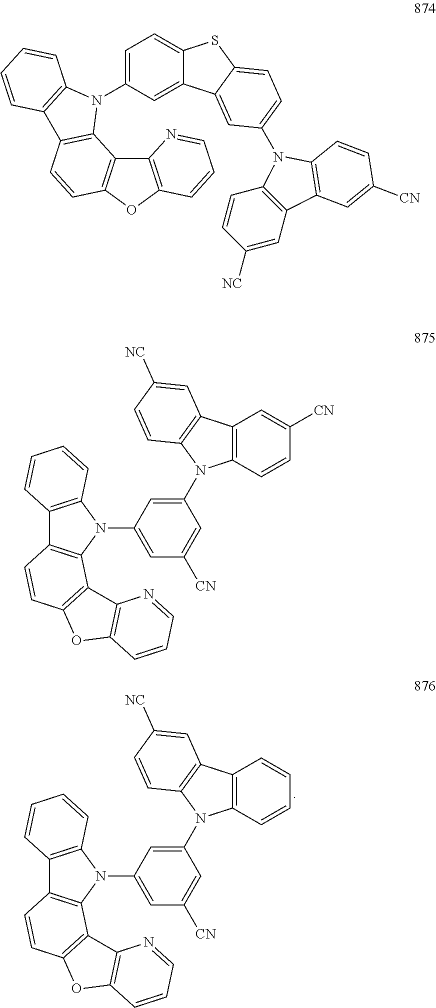

15. An organic light-emitting device comprising:
a first electrode;
a second electrode; and
an organic layer disposed between the first electrode and the second electrode,
wherein the organic layer comprises an emission layer, and
wherein the organic layer comprises at least one of the condensed cyclic compounds represented by Formula 1 of claim 1.

16. The organic light-emitting device of claim 15, wherein
the first electrode is an anode,
the second electrode is a cathode, and
the organic layer comprises a hole transport region disposed between the first electrode and the emission layer and an electron transport region disposed between the emission layer and the second electrode, wherein
the hole transport region comprises at least one selected from a hole injection layer, a hole transport layer, and an electron blocking layer,
the electron transport region comprises at least one selected from a hole blocking layer, an electron transport layer, and an electron injection layer.

17. The organic light-emitting device of claim 15, wherein
the emission layer comprises the condensed cyclic compound represented by Formula 1.

18. The organic light-emitting device of claim 15, wherein
the emission layer comprises a host and a dopant,
the host comprises a condensed cyclic compound represented by Formula 1, and
an amount of the host is larger than an amount of the dopant.

19. The organic light-emitting device of claim 18, wherein
the emission layer emits blue light.

20. The organic light-emitting device of claim 16, wherein
the electron transport region comprises the hole blocking layer, and
the hole blocking layer comprises the condensed cyclic compound represented by Formula 1.
